(19) United States
(12) Patent Application Publication
(10) Pub. No.: US 2026/0317366 A1
(43) Pub. Date: Oct. 15, 2026

(54) LYMPHATIC SYSTEM-TARGETING COMPOUNDS

(71) Applicant: Sage Therapeutics, LLC, Wilmington, DE (US)

(72) Inventors: Jason Legassie, Wilmington, DE (US); Matthew D. Hill, Wilmington, DE (US); Michael C. Quirk, Wilmington, DE (US); Andrew Anthony Martins, Wilmington, DE (US)

(21) Appl. No.: 19/480,233

(22) PCT Filed: May 2, 2024
(86) PCT No.: PCT/US2024/027554

§ 371 Date: Oct. 30, 2025

Related U.S. Application Data
(60) Provisional application No. 63/463,540, filed on May 2, 2023.
(60) Provisional application No. 63/631,365, filed on Apr. 8, 2024.

(51) Int. Cl. C07J 9/00; A61K 31/575; C07J 17/00; C07J 41/00

(52) U.S. Cl. CPC C07J 9/00; A61K 31/575; C07J 17/00; C07J 41/0055

(57) ABSTRACT

The disclosure provides lymphatic system-targeting compounds of Formula (I), and pharmaceutically acceptable salts thereof, pharmaceutical compositions thereof and and methods of treating various conditions associated with NMDA receptor modulation.

### CROSS REFERENCE TO RELATED APPLICATIONS

This application claims priority to and the benefit of U.S. Provisional Application No. 63/463,540, filed May 2, 2023, and U.S. Provisional Application No. 63/631,365, filed Apr. 8, 2024, the disclosure of each of which is incorporated by reference herein in its entirety.

### BACKGROUND

NMDA receptors are highly expressed in the CNS and are involved in excitatory synaptic transmission. Activating these receptors contributes to synaptic plasticity in some circumstances and excitotoxicity in others. These receptors are ligand-gated ion channels that admit Ca²⁺ after binding of the neurotransmitters glutamate and glycine, and are fundamental to excitatory neurotransmission and normal CNS function. NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. Positive modulators may be useful as therapeutic agents with potential clinical uses as cognitive enhancers and in the treatment of psychiatric disorders in which glutamatergic transmission is reduced or defective (see, e.g., Horak et al., J. Neuroscience, 2004, 24(46), 10318-10325). In contrast, negative modulators may be useful as therapeutic agents with potential clinical uses in the treatment of psychiatric disorders in which glutamatergic transmission is pathologically increased (e.g., treatment resistant depression).

The lymphatic system is a network of vessels, organs (e.g., bone marrow, thymus, lymph nodes and spleen) and tissues distributed throughout the body that work together to move lymphatic fluid back into the circulatory system. The lymphatic system has several key functions including transporting immune cells, collecting excess fluid from the body's tissues and returning it to the bloodstream, filtering out waste products and abnormal cells, absorbing dietary lipids, and tumor metastasis. These features of the lymphatic system have made it a desirable approach for improving drug delivery to target organs. For example, drug delivery via the lymphatic system has several major advantages, including circumventing first-pass metabolism in the liver and targeting drugs to tissues via the lymphatic system (e.g., certain types of cancer and human immunodeficiency virus). Lipid-based therapeutics have unique characteristics that make them promising candidates for enhanced lymphatic delivery.

Therefore, there remains a need to develop therapeutics that facilitate the delivery of NMDA receptor modulators via the lymphatic system for treating CNS conditions. The compounds, compositions and methods described herein are directed toward this end.

### SUMMARY OF THE INVENTION

Described herein are compounds of Formula (I), pharmaceutical compositions comprising them and methods of use thereof. Also described herein is the use of pharmaceutical compositions comprising the compounds of Formula (I) for the manufacture of a medicament for the treatment of diseases or conditions associated with NMDA receptor modulation. Further described herein are pharmaceutical compositions comprising compounds of Formula (I) for use in treating diseases or conditions associated with NMDA receptor modulation.

In one aspect, the disclosure provides a compound of Formula (I):

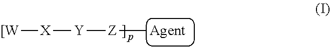

or a pharmaceutically acceptable salt thereof.

In another aspect, the present disclosure provides a pharmaceutical composition comprising a compound of Formula (I), or a pharmaceutically acceptable salt thereof, and a pharmaceutically acceptable carrier thereof.

In another aspect, the present disclosure provides a method of modulating an NMDA receptor in a subject, comprising administering to the subject an effective amount of a compound of Formula (I), or a pharmaceutically acceptable salt thereof.

In another aspect, the present disclosure provides a method of treating a disease, disorder, or condition requiring allosteric NMDA receptor modulation in a subject, comprising administering to the subject an effective amount of a compound of Formula (I), or a pharmaceutically acceptable salt thereof or a pharmaceutical composition thereof.

In another aspect, the present disclosure provides a compound of Formula (I), or a pharmaceutically acceptable salt thereof for use in modulating an NMDA receptor in a subject.

In another aspect, the present disclosure provides a composition comprising a compound of Formula (I), or a pharmaceutically acceptable salt thereof for use in modulating an NMDA receptor in a subject.

In another aspect, the present disclosure provides a compound of Formula (I), or a pharmaceutically acceptable salt thereof for use in treating a disease, disorder, or condition requiring allosteric NMDA receptor modulation.

In another aspect, the present disclosure provides a composition comprising a compound of Formula (I), or a pharmaceutically acceptable salt thereof for use in treating a disease, disorder, or condition requiring allosteric NMDA receptor modulation.

In another aspect, the present disclosure provides the use of a compound of Formula (I), or a pharmaceutically acceptable salt thereof for the manufacture of a medicament for allosterically modulating an NMDA receptor.

In another aspect, the present disclosure provides the use of a compound of Formula (I), or a pharmaceutically acceptable salt thereof for the manufacture of a medicament for treating a disease, disorder, or condition requiring allosteric NMDA receptor modulation.

### DETAILED DESCRIPTION OF THE INVENTION

### General Definitions

The term “herein” means the entire application.

Unless otherwise defined herein, scientific and technical terms used in this application shall have the meanings that are commonly understood by those of ordinary skill in the art to which this disclosure belongs. Generally, nomenclature used in connection with the compounds, composition and methods described herein, are those well-known and commonly used in the art.

It should be understood that any of the embodiments described herein, including those described under different embodiments of the disclosure and different parts of the specification (including embodiments described only in the Examples) can be combined with one or more other embodiments of the disclosure, unless explicitly disclaimed or improper. Combination of embodiments are not limited to those specific combinations claimed via the multiple dependent claims. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range and including the endpoints, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

All of the publications, patents and published patent applications referred to in this application are specifically incorporated by reference herein. In case of conflict, the present specification, including its specific definitions, will control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers, e.g., stereoisomers, can be isolated from mixtures by methods known to those skilled in the art, including chiral high-performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

As used herein, a pure enantiomeric compounds substantially free from other enantiomers or stereoisomers of the compound (i.e., in enantiomeric excess). In other words, an “S” form of the compounds substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound. As used herein, the term “diastereomeric purity” refers to the amount of a compound having the depicted absolute stereochemistry, expressed as a percentage of the total amount of the depicted compound and its diastereomers. The term “diastereomerically pure” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the diastereomer. Methods for determining diastereomeric and enantiomeric purity are well-known in the art. Diastereomeric purity can be determined by any analytical method capable of quantitatively distinguishing between a compound and its diastereomers, such as high-performance liquid chromatography (HPLC) or supercritical fluid chromatograph (SFC).

In the compositions provided herein, an enantiomerically pure compound can be present with other active or inactive ingredients. For example, a pharmaceutical composition comprising enantiomerically pure (R)-position/center/carbon compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present disclosure. It should also be understood that when described herein any of the moieties defined herein may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

“Aliphatic” refers to an alkyl, alkenyl, alkynyl, or carbocyclyl group, as defined herein. A “bivalent aliphatic” refers to a bivalent radical of an alkyl (i.e., alkylene), alkenyl (i.e., alkenylene), alkynyl (i.e., alkynylene), or carbocyclyl group.

“Alkyl” refers to a radical of a straight-chain or branched saturated hydrocarbon group comprising from 1 to 50 carbon atoms (“C₁₋₅₀ alkyl”). In some embodiments, an alkyl group has 1 carbon atom (“C₁ alkyl”). In some embodiments, an alkyl group has 1 to 2 carbon atoms (“C₁₋₂ alkyl”). In some embodiments, an alkyl group has 1 to 3 carbon atoms (“C₁₋₃ alkyl”). In some embodiments, an alkyl group has 1 to 4 carbon atoms (“C₁₋₄ alkyl”). In some embodiments, an alkyl group has 1 to 5 carbon atoms (“C₁₋₅ alkyl”). In some embodiments, an alkyl group has 1 to 6 carbon atoms (“C₁₋₆ alkyl”), and so on. Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 4 substituents, 1 to 3 substituents, or 1 substituent. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

As used herein, “alkylene,” “alkenylene,” “alkynylene,” “heteroalkylene,” “heteroalkenylene,” and “heteroalkynylene,” refer to a bivalent radical of an alkyl, alkenyl, alkynyl, heteroalkyl, heteroalkenyl, and heteroalkynyl group, respectively. When a range or number of carbons is provided for a particular “alkylene,” “alkenylene,” “alkynylene,” “heteroalkylene,” “heteroalkenylene,” or “heteroalkynylene,” group, it is understood that the range or number refers to the range or number of carbons in the linear carbon bivalent chain. “Alkylene,” “alkenylene,” “alkynylene,” “heteroalkylene,” “heteroalkenylene,” and “heteroalkynylene” groups may be substituted or unsubstituted with one or more substituents as described herein.

“Alkylene” or “alkylene group” refers to an alkyl group wherein two hydrogens are removed to provide a bivalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more halo, —NO₂, —OH, C₁-C₆ alkoxy, C₁-C₆ alkyl (e.g., methyl) groups, including but not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), or C₁-C₆ cycloalkyl, and the like. Alkylene abbreviations include, but are not limited to, —(CH(CH₃))—, —(CH(CH₂CH₃))—, —(CH(CH₂CH₂CH₃))—, —(CH(CH₂CH₂CH₂CH₃))—, —(CH₂CH(CH₂CH₂CH₂CH₃))—, —(CH₂CH₂CH(CH₂CH₂CH₂CH₃))—, —(CH(CH₃)CH₂)—, —(CH(CH₃)CH₂CH₂)—, —(CH(CH₃)CH₂CH₂CH₂)—, —(CH₂CH(CH₃)CH₂)—, —(CH₂CH(CH₃)CH₂CH₂)—, and —(CH₂CH₂CH(CH₃)CH₂CH₂)—.

“Alkenyl” refers to a radical of a straight-chain or branched hydrocarbon group comprising from 2 to 50 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₅₀ alkenyl”). In certain embodiments, alkenyl does not contain any triple bonds. In some embodiments, an alkenyl group has 2 carbon atoms (“C₂ alkenyl”). In some embodiments, an alkenyl group has 2 to 3 carbon atoms (“C₂₋₃ alkenyl”). In some embodiments, an alkenyl group has 2 to 4 carbon atoms (“C₂₋₄ alkenyl”). In some embodiments, an alkenyl group has 2 to 5 carbon atoms (“C₂₋₅ alkenyl”). In some embodiments, an alkenyl group has 2 to 6 carbon atoms (“C₂₋₆ alkenyl”). In some embodiments, an alkenyl group has 2 to 7 carbon atoms (“C₂₋₇ alkenyl”). In some embodiments, an alkenyl group has 2 to 8 carbon atoms (“C₂₋₈ alkenyl”). In some embodiments, an alkenyl group has 2 to 9 carbon atoms (“C₂₋₉ alkenyl”). In some embodiments, an alkenyl group has 2 to 10 carbon atoms (“C₂₋₁₀ alkenyl”), and so on. The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₅), octatrienyl (C₅), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

“Alkenylene” refers to an alkenyl group wherein two hydrogens are removed to provide a bivalent radical, and which may be substituted or unsubstituted. Exemplary unsubstituted bivalent alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” refers to a radical of a straight-chain or branched hydrocarbon group comprising from 2 to 50 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₅₀ alkynyl”). In some embodiments, an alkynyl group has 2 carbon atoms (“C₂ alkynyl”). In some embodiments, an alkynyl group has 2 to 3 carbon atoms (“C₂₋₃ alkynyl”). In some embodiments, an alkynyl group has 2 to 4 carbon atoms (“C₂₋₄ alkynyl”). In some embodiments, an alkynyl group has 2 to 5 carbon atoms (“C₂₋₅ alkynyl”). In some embodiments, an alkynyl group has 2 to 6 carbon atoms (“C₂₋₆ alkynyl”). In some embodiments, an alkynyl group has 2 to 7 carbon atoms (“C₂₋₇ alkynyl”). In some embodiments, an alkynyl group has 2 to 8 carbon atoms (“C₂₋₈ alkynyl”). In some embodiments, an alkynyl group has 2 to 9 carbon atoms (“C₂₋₉ alkynyl”). In some embodiments, an alkynyl group has 2 to 10 carbon atoms (“C₂₋₁₀ alkynyl”), and so on. In certain embodiments, alkynyl does not contain any double bonds. The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers to a linear alkynyl group wherein two hydrogens are removed to provide a bivalent radical, and which may be substituted or unsubstituted. Exemplary bivalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group comprising from 1 to 10 carbon atoms and 1, 2, 3, or 4 heteroatoms (“heteroC₁₋₁₀ alkyl”). In some embodiments, a heteroalkyl group is a saturated group comprising 1 to 9 carbon atoms and 1, 2, 3, or 4 heteroatoms (“heteroC₁₋₉ alkyl”). In some embodiments, a heteroalkyl group is a saturated group comprising 1 to 8 carbon atoms and 1, 2, 3, or 4 heteroatoms (“heteroC₁₋₈ alkyl”). In some embodiments, a heteroalkyl group is a saturated group comprising 1 to 7 carbon atoms and 1, 2, 3, or 4 heteroatoms (“heteroC₁₋₇ alkyl”). In some embodiments, a heteroalkyl group is a group comprising 1 to 6 carbon atoms and 1, 2, or 3 heteroatoms (“heteroC₁₋₆ alkyl”). In some embodiments, a heteroalkyl group is a saturated group comprising 1 to 5 carbon atoms and 1 or 2 heteroatoms (“heteroC₁₋₅ alkyl”). In some embodiments, a heteroalkyl group is a saturated group comprising 1 to 4 carbon atoms and lor 2 heteroatoms (“heteroC₁₋₄ alkyl”). In some embodiments, a heteroalkyl group is a saturated group comprising 1 to 3 carbon atoms and 1 heteroatom (“heteroC₁ 3 alkyl”). In some embodiments, a heteroalkyl group is a saturated group comprising 1 to 2 carbon atoms and 1 heteroatom (“heteroC₁₋₂ alkyl”). In some embodiments, a heteroalkyl group is a saturated group comprising 1 carbon atom and 1 heteroatom (“heteroC₁ alkyl”). In some embodiments, a heteroalkyl group is a saturated group comprising 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group comprising from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and lor 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkynyl group refers to a group comprising from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆ alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and lor 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

“Aryl” refers to a radical of a monocyclic or polycyclic (e.g., bicyclic or tricyclic) 4n+2 aromatic ring system (e.g., having 6, 10, or 14 π electrons shared in a cyclic array) comprising 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). Aryl also includes ring systems wherein the aryl ring, as defined herein, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following:

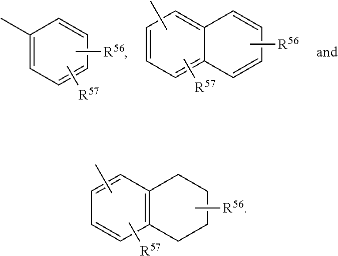

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. Heteroaryl includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. Heteroaryl also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively.

Examples of representative heteroaryls include the following:

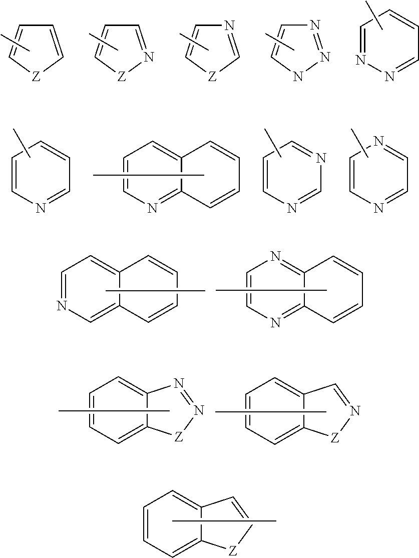

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl,” “carbocyclic” or “carbocycle” refers to a radical of a non-aromatic cyclic hydrocarbon group comprising from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl,” “carbocycle” or “carbocyclic” refers to a monocyclic, saturated carbocyclyl group comprising from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C_{5-_}cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₅). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl,” “heterocycle” or “heterocyclic” refers to a radical of a 3- to 10-membered non-aromatic ring system comprising ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system comprising ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system comprising ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system comprising ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like comprising from 1 to 5, and particularly from 1 to 3 heteroatoms.

“Acyl” refers to a radical —C(O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein.

“Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)(C₃-C₁₀ cycloalkyl), and —C(O)—(CH₂)(4-10 membered heterocyclyl), wherein t is an integer from 0 to 4. In certain embodiments, R²⁰ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)₁(C₆-C₁₀ aryl), —O—(CH₂)ₜ(5-10 membered heteroaryl), —O—(CH₂)₁(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)(C₃-C₁₀ cycloalkyl), and —NR³⁹—(CH₂)(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Carboxy” refers to the radical —C(O)OH.

“Cyano” refers to the radical —CN.

“Halo,” or “halogen,” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

A “wavy bond” or “ [symbol] ” in the structures depicted herein refers to the point of attachment of the group.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{b}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ~NNR^{bb}C(═O)R^{bb}, ═NNR^{bb}C(═O)OR^{aa}, ═NNR^{bb}S(═O)₂R^{bb}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ee}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{cc}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁-alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound disclosed herein that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

“Pharmaceutically acceptable carrier” refers to compositions, carriers, diluents, and reagents which are pharmaceutically acceptable materials that are capable of administration to or upon a subject. A pharmaceutically acceptable carrier can be involved with carrying or transporting the subject agents from one organ, or portion of the body, to another organ, or portion of the body. The carrier can be in the form of a solid, semi-solid or liquid diluent, cream or a capsule. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof.

“Isotopic variant” refers to a compound disclosed herein (e.g., a compound of Formula (I) or a pharmaceutically acceptable salt thereof), wherein one or more atoms is replaced by an atom having the same atomic number, but an atomic mass or mass number different from the atomic mass or mass number usually found in nature. Examples of isotopes that can be incorporated into compounds of the present application include, but are not limited to, isotopes of hydrogen, carbon, nitrogen, oxygen, phosphorous, sulfur, fluorine, and chlorine, such as ²H, ³H, ¹³C, ¹⁴C, ¹⁵N, ¹⁷O, ¹⁸O, ³²P, ³³P, ³³S, ³⁴S, ³⁵S, ¹⁸F, and ³⁷Cl. Compounds disclosed herein which contain the aforementioned isotopes and/or other isotopes of other atoms are within the scope of this disclosure. Certain isotopic variants of the compounds of the present application, for example, those into which radioactive isotopes (e.g., ³H and ¹⁴C) are incorporated, may be useful in drug and/or substrate distribution assays. Further, substitution with heavier isotopes (e.g., ²H) can afford certain therapeutic advantages resulting from greater metabolic stability, for example, increased in vivo half-life or reduced dosage requirements and, hence, may be preferred in some circumstances. Isotopic variants of compounds and pharmaceutically acceptable salts thereof disclosed herein can generally be prepared by carrying out the procedures disclosed in the Schemes and/or in the Examples, by substituting a readily available isotopically-labeled reagent for a non-isotopically-labeled reagent.

A “subject” to which administration is contemplated includes, but is not limited to, a human subject (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, the term “treat,” “treating,” or “treatment” includes reversing, reducing, or arresting the symptoms, clinical signs, and underlying pathology of a condition in a manner to improve or stabilize a subject's condition. As used herein, and as well understood in the art, “treatment” is an approach for obtaining beneficial or desired results, including clinical results. Beneficial or desired clinical results can include, but are not limited to, alleviation, amelioration, reduction of the severity, or slowing the progression of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, and unless otherwise specified, the term “prophylactic,” “prevention,” and variations thereof, contemplates an action that occurs before a subject begins to suffer from the specified disease, disorder, or condition.

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

The terms “pharmaceutically effective amount,” “therapeutically effective amount,” and “therapeutically effective dose” are used interchangeably herein and refer to an amount sufficient to treat a disease in a patient, e.g., effecting a beneficial and/or desirable alteration in the health of a patient suffering from a disease, treatment, healing, inhibition or amelioration of a physiological response or condition, delaying or minimizing one or more symptoms associated with the disease, disorder, or condition etc. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The terms “pharmaceutically effective amount,” “therapeutically effective amount,” or “therapeutically effective dose” also refer to the amount required to improve the clinical symptoms of a patient. A therapeutically effective amount of a compound also refers to an amount of the therapeutic agent, alone or in combination with other therapies, which provides a therapeutic benefit in the treatment of the disease, disorder, or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder, or condition, or one or more symptoms associated with the disease, disorder, or condition, or prevent its recurrence. A prophylactically effective amount of a compound means an amount of a therapeutic agent, alone or in combination with other agents, which provides a prophylactic benefit in the prevention of the disease, disorder, or condition. The term “prophylactically effective amount” can encompass an amount that improves overall prophylaxis or enhances the prophylactic efficacy of another prophylactic agent.

As used herein, and unless otherwise specified, “pharmacokinetics” can be defined as the study of bodily absorption, distribution, metabolism, and excretion of drugs. “Pharmacokinetics” can also be defined as the characteristic interactions of a drug and a body in terms of its absorption, distribution, metabolism, and excretion; or a branch of pharmacology concerned with the way drugs are taken into, move around, and are eliminated from, a body.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another, and/or who provides a patient with a prescription for a drug is administering the drug to the patient. When a method is part of a therapeutic regimen involving more than one agent or treatment modality, the disclosure contemplates that the agents may be administered at the same or differing times and via the same or differing routes of administration. Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g., solubility, digestibility, bioavailability, stability, and toxicity).

The term “lipid,” as used herein, refers to natural and non-natural hydrophobic and/or lipophilic fats, oils, polymers, hydrocarbons, and other such materials. In some embodiments, suitable lipids, when incorporated into a compound, are processed or metabolized similarly to triglycerides in the gastrointestinal tract or mimic such processing or metabolism. The term “glyceride” refers to an ester of glycerol (1,2,3-propanetriol) with acyl radicals of fatty acids or other lipids and is also known as an acylglycerol. A “monoglyceride” is a glycerol molecule wherein only one position of the molecule is esterified with a fatty acid. A “diglyceride” is a glycerol molecule wherein two positions of the molecule are esterified with fatty acids. A “diglyceride” is a glycerol molecule wherein three positions of the molecule are esterified with fatty acids. A “simple glyceride” is one where all esterified positions contain the same fatty acid. A “mixed glyceride” is one where different fatty acids are present at the esterified positions. The carbons of the glycerol backbone are designated sn-1, sn-2 and sn-3, with sn-2 being in the middle and sn-1 and sn-3 being the ends of the glycerol.

The term “cleavable moiety” refers to a chemical moiety that may be cleaved via hydrolysis, reduction, or enzymatic reaction. Cleavable moieties include, but are not limited to acid-labile moieties, hydrolysis-labile moieties, enzymatically cleavable moieties, reduction labile moieties, and self-immolative moieties.

The term “self-immolative moiety,” as used herein, refers to a bivalent chemical moiety that comprises a covalent, scissile bond as one of its bivalent bonds and a stable, covalent bond with a therapeutic agent as its other bivalent bond, wherein the bond with the therapeutic agent becomes labile upon cleavage of the scissile bond. The self-immolative group can be any such group known to those of skill in the art. Examples of self-immolative moieties include, but are not limited to, disulfide groups, hydrazones, acetal self-immolative moieties, carboxyacetal self-immolative moieties, carboxy(methylacetal) self-immolative moieties, para-hydroxybenzyl carbonyl self-immolative moieties, flipped ester self-immolative moieties, and trimethyl lock, or 2-hydroxyphenyl carbamate (2-HIPC) self-immolative moieties. Other suitable self-immolative moieties are known in the art as described, for example, in C. Blencowe et al., Polym. Chem. 2011, 2, 773-790 and Kratz et al., ChemMedChem. 2008, 3(1), 20-53; Huvelle et al., Org. Biomol. Chem. 2017, 15(16), 3435-3443; and Alouane. et al., Angewandte Chemie International Edition 2015, 54 (26), 7492-7509; and Levine et al., Chem. Sci. 2012 3(8), 2412-2420; each of which is hereby incorporated by reference in its entirety.

The term “acid labile” refers to a molecule or compound that is sensitive to acids and can be cleaved or undergo significant changes in its structure or properties when exposed to acidic conditions.

As generally described herein, the disclosure provides compounds useful for preventing and/or treating a broad range of disorders, including, but not limited to, NMDA-mediated disorders. These compounds are expected to show, inter alia, improved in vivo potency, pharmacokinetic (PK) properties, oral bioavailability, formulatability, stability, and/or safety.

### Compounds

In one aspect, the disclosure provides a compound of Formula (I):

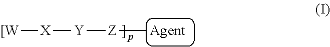

or a pharmaceutically acceptable salt thereof, wherein:
W is

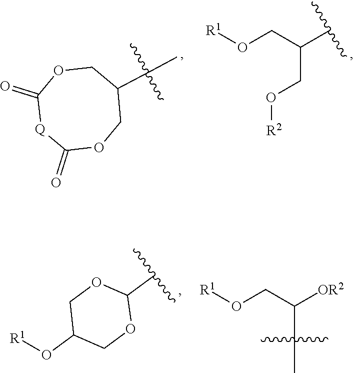

or —C(O)N(R³)₂;
Q is —C₁₋₈alkylene-;
each of R¹ and R² is independently hydrogen, an acid-labile group, a lipid, or —C(O)R³;
each R³ is independently an optionally substituted C₁₋₄₀ aliphatic;
X is —O—, —NR—, —S—, —O(C₁₋₆ aliphatic)-O—, —O(C₁₋₆ aliphatic)-S—, —O(C₁₋₆ aliphatic)-NR—, —S(C₁₋₆ aliphatic)-O—, —S(C₁₋₆ aliphatic)-S—, —S(C₁₋₆ aliphatic)-NR—, —NR(C₁₋₆ aliphatic)-O—, —NR(C₁₋₆ aliphatic)-S—, —NR(C₁₋₆ aliphatic)-NR—, or —(C₁₋₆ aliphatic)-, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen;
each R is independently hydrogen, or an optionally substituted group selected from the group consisting of C₁₋₆ aliphatic, 3-8 membered carbocycle, C₆₋₁₀aryl, 4-8 membered heterocycle comprising 1-2 heteroatoms independently selected from the group consisting of N, O and S, and 5-10 membered heteroaryl comprising 1-4 heteroatoms independently selected from the group consisting of N, O, and S;
Y is absent, —C(O)—, —C(NR)—, or —C(S)—;
Z is absent or an optionally substituted bivalent C₁₋₃₀ aliphatic, wherein 0-8 methylene units of Z are independently replaced by —R⁸—, —O—, —NR—, —S—, —OC(O)—, —C(O)O—, —C(O)—, —S(O)—, —S(O)₂—, —C(S)—, —OS(O)₂—, —S(O)₂O—, —NRS(O)₂—, —S(O)₂NR—, —NRC(O)—, —C(O)NR—, —OC(O)NR—, —NRC(O)O—, or an amino acid; and wherein one methylene unit of Z is optionally replaced with -M-; or
Z is selected from the group consisting of —C(R^{4a})(R^{4b})C(O)-M-, C(R^{4a})(R^{4b})-M-, —C(R^{4a})(R^{4b})C(R^{5a})(R^{5b})(CH₂)ₙC(O)-M-, —C(R^{4a})(R^{4b})C(R^{5a})(R^{5b})(CH₂)ₙ-M-, —(CH₂)ₘC(R^{4a})(R^{4b})(CH₂)ₙC(R^{5a})(R^{5b})(CH₂)ₘC(O)-M-, and —(CH₂)ₘC(R^{4a})(R^{4b})(CH₂)ₙC(R^{5a})(R^{5b})(CH₂)ₘ-M-;
   wherein either side of Z may be attached to Agent;
each —R⁸— is independently an optionally substituted bivalent group selected from the group consisting of C₃₋₆ carbocycle, C₆₋₁₀ aryl, 3-6 membered heterocycle comprising 1-4 heteroatoms independently selected from the group consisting of N, O, and S, and 5-10 membered heteroaryl comprising 1-4 heteroatoms independently selected from the group consisting of N, O, and S;
each R^{4a}, R^{4b}, R^{5b} and R^{5b} is independently selected from the group consisting of hydrogen, deuterium, halogen, —CN, —OR, —NR₂, —SR, —R⁹, and —R¹⁰; or
R^{4a} and R^{4b} or R^{5a} and R^{5b} together with the carbon atom to which they are attached, form a C₃₋₆ carbocycle or a 3-6 membered heterocycle comprising 1-2 heteroatoms independently selected from the group consisting of N, O and S;
each R⁹ is independently selected from the group consisting of 3-8 membered carbocycle, C₆₋₁₀aryl, 4-8 membered heterocycle comprising 1-2 heteroatoms independently selected from the group consisting of N, O, and S, and 5-10 membered heteroaryl comprising 1-4 heteroatoms independently selected from the group consisting of nitrogen, oxygen, and sulfur;
each R¹⁰ is independently selected from the group consisting of C₁₋₆ aliphatic optionally substituted with 1 to 6 —CN, —OR, —NR₂, —SR, —R⁹, deuterium or halogen;
-M- is a cleavable moiety;
each n is independently 0-18;
each m is independently 0-6;
p is 1 or 2; and
   Agent is selected from the group consisting of:

| Agent | |
|---|---|
| No. | Structure |
| 1 | 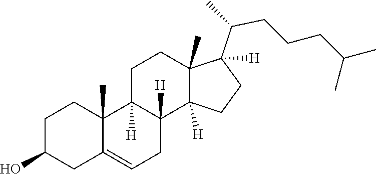 |
| 2 | 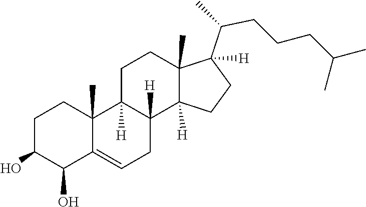 |
| 3 | 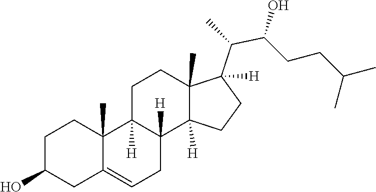 |
| 4 | 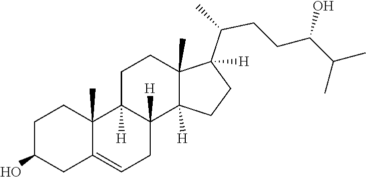 |
| 5 | 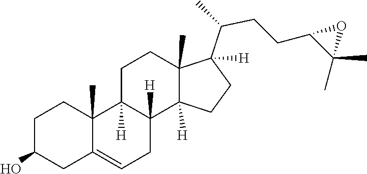 |
| 6 | 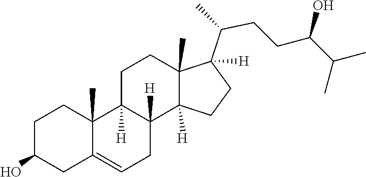 |
| 7 | 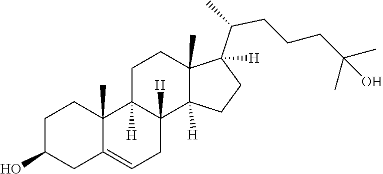 |
| 8 | 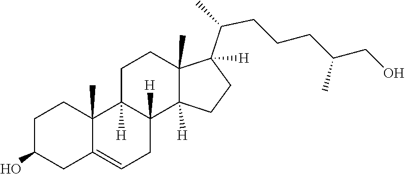 |
| 9 | 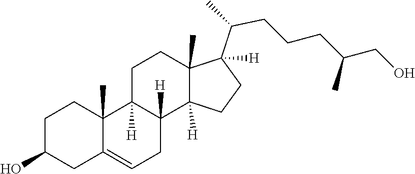 |
| 10 | 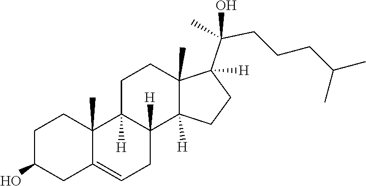 |
| 11 | 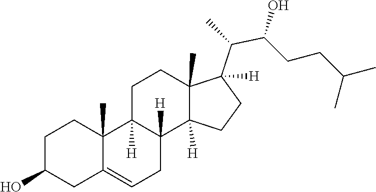 |
| 12 | 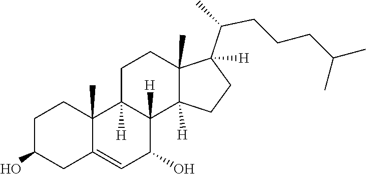 |
| 13 | 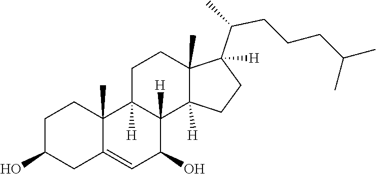 |
| 14 | 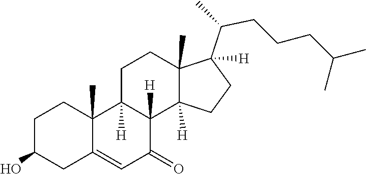 |
| 15 | 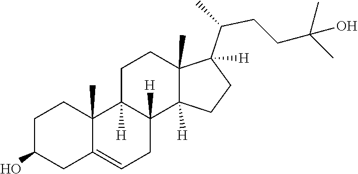 |
| 16 | 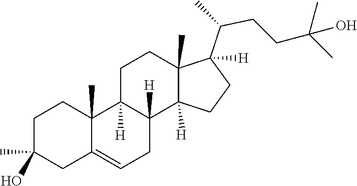 |
| 17 | 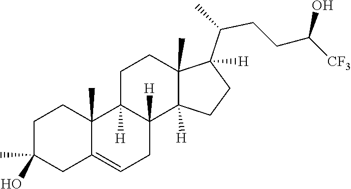 |
| 18 | 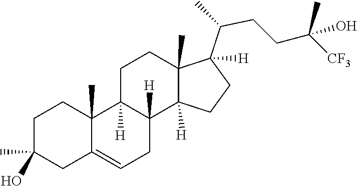 |
| 19 | 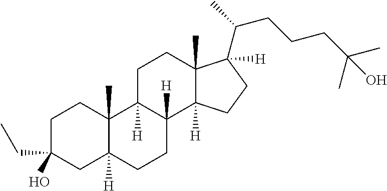 |
| 20 | 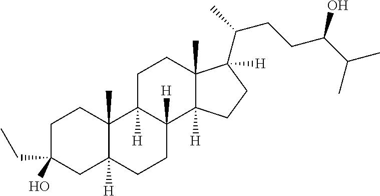 |
| 21 | 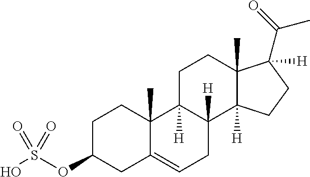 |
| 22 | 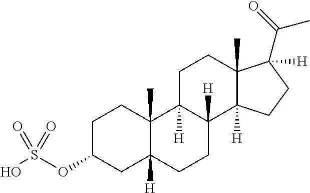 |
| 23 | 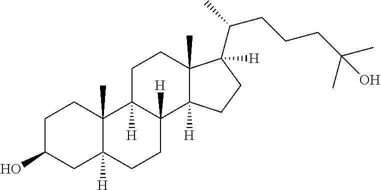 |
| 24 | 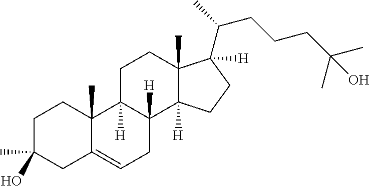 |
| 25 | 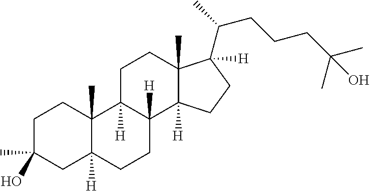 |
| 26 | 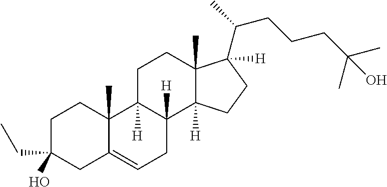 |
| 27 | 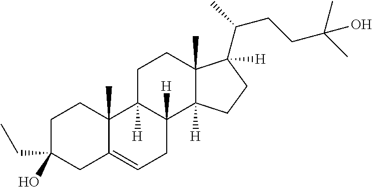 |

and deuterated variants thereof; wherein the point(s) of attachment of Agent to Z replaces one or more hydrogen atoms on the Agent.

In some embodiments, the disclosure provides a compound of Formula (I):

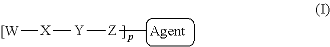

or a pharmaceutically acceptable salt thereof, wherein:
W is

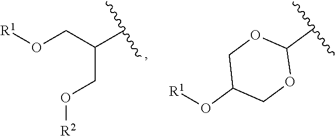

or —C(O)N(R³)₂
each of R¹ and R² is independently hydrogen, an acid-labile group, a lipid, or —C(O)R³;
each R³ is independently an optionally substituted C₁₋₄₀ aliphatic;
X is —O—, —NR—, —S—, —O(C₁₋₆ aliphatic)-O—, —O(C₁₋₆ aliphatic)-S—, —O(C₁₋₆ aliphatic)-NR—, —S(C₁₋₆ aliphatic)-O—, —S(C₁₋₆ aliphatic)-S—, —S(C₁₋₆ aliphatic)-NR—, —NR(C₁₋₆ aliphatic)-O—, —NR(C₁₋₆ aliphatic)-S—, —NR(C₁₋₆ aliphatic)-NR—, or —(C₁₋₆ aliphatic)-, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen;
each R is independently hydrogen, or an optionally substituted group selected from the group consisting of C₁₋₆ aliphatic, 3-8 membered carbocycle, C₆₋₁₀aryl, 4-8 membered heterocycle comprising 1-2 heteroatoms independently selected from the group consisting of N, O and S, and 5-10 membered heteroaryl comprising 1-4 heteroatoms independently selected from the group consisting of N, O, and S;
Y is absent, —C(O)—, —C(NR)—, or —C(S)—;
Z is absent or an optionally substituted bivalent C₁₋₃₀ aliphatic, wherein 0-8 methylene units of Z are independently replaced by —R⁸—, —O—, —NR—, —S—, —OC(O)—, —C(O)O—, —C(O)—, —S(O)—, —S(O)₂—, —C(S)—, —OS(O)₂—, —S(O)₂O—, —NRS(O)₂—, —S(O)₂NR—, —NRC(O)—, —C(O)NR—, —OC(O)NR—, —NRC(O)O—, or an amino acid; and wherein one methylene unit of Z is optionally replaced with -M-; or
Z is selected from the group consisting of —C(R^{4a})(R^{4b})C(O)-M-, C(R^{4a})(R^{4b})-M-, —C(R^{4a})(R^{4b})C(R^{5a})(R^{5b})(CH₂)~C(O)-M-, —C(R^{4a})(R^{4b})C(R^{5a})(R^{5b})(CH₂)ₙ-M-, —(CH₂)ₘC(R^{4a})(R^{4b})(CH₂)ₙC(R^{5a})(R^{5b})(CH₂)ₘC(O)-M-, and —(CH₂)ₘC(R^{4a})(R^{4b})(CH₂)ₙC(R^{5a})(R^{5b})(CH₂)ₘ-M-;
   wherein either side of Z may be attached to Agent;
each —R^{g}— is independently an optionally substituted bivalent group selected from the group consisting of C₃₋₆ carbocycle, C₆₋₁₀ aryl, 3-6 membered heterocycle comprising 1-4 heteroatoms independently selected from the group consisting of N, O, and S, and 5-10 membered heteroaryl comprising 1-4 heteroatoms independently selected from the group consisting of N, O, and S;
each R^{4a}, R^{4b}, R^{5b} and R^{5b} is independently selected from the group consisting of hydrogen, deuterium, halogen, —CN, —OR, —NR₂, —SR, —R⁹, and —R¹⁰; or
R^{4a} and R^{4b} or R^{5a} and R^{5b} together with the carbon atom to which they are attached, form a C₃₋₆ carbocycle or a 3-6 membered heterocycle comprising 1-2 heteroatoms independently selected from the group consisting of N, O and S;
each R⁹ is independently selected from the group consisting of 3-8 membered carbocycle, C₆₋₁₀aryl, 4-8 membered heterocycle comprising 1-2 heteroatoms independently selected from the group consisting of N, O, and S, and 5-10 membered heteroaryl comprising 1-4 heteroatoms independently selected from the group consisting of nitrogen, oxygen, and sulfur;
each R¹⁰ is independently selected from the group consisting of C₁₋₆ aliphatic optionally substituted with 1 to 6 —CN, —OR, —NR₂, —SR, —R⁹, deuterium or halogen;
-M- is a cleavable moiety;
each n is independently 0-18;
each m is independently 0-6;
p is 1 or 2; and
   Agent is selected from the group consisting of:

| Agent | |
|---|---|
| No. | Structure |
| 1 | 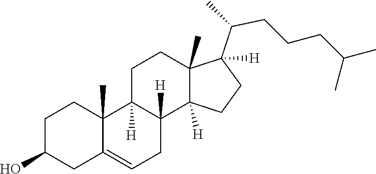 |
| 2 | 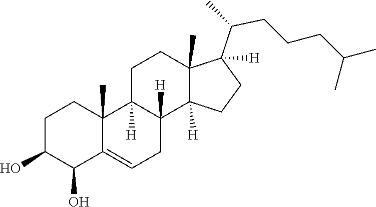 |
| 3 | 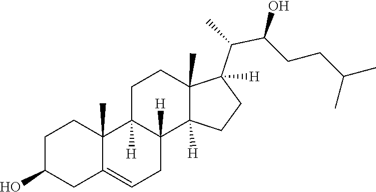 |
| 4 | 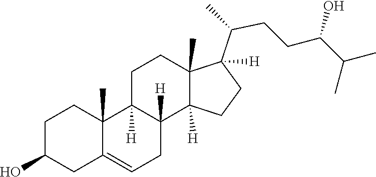 |
| 5 | 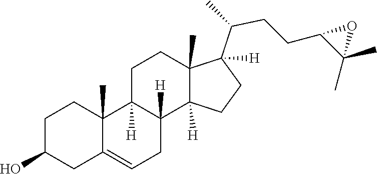 |
| 6 | 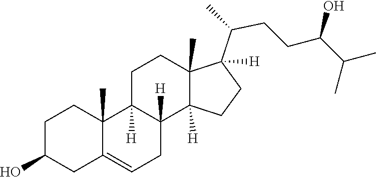 |
| 7 | 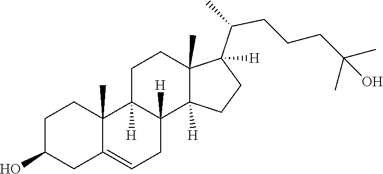 |
| 8 | 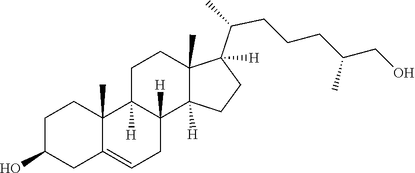 |
| 9 | 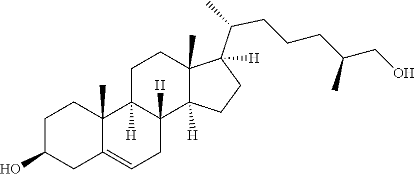 |
| 10 | 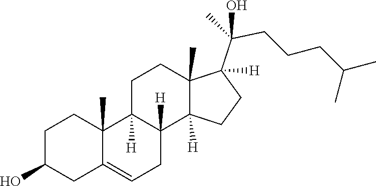 |
| 11 | 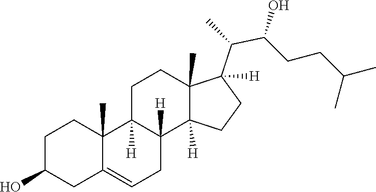 |
| 12 | 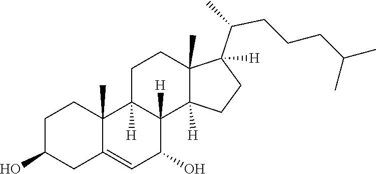 |
| 13 | 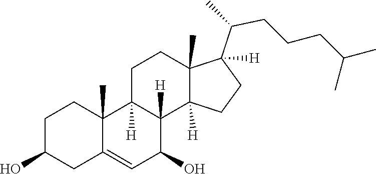 |
| 14 | 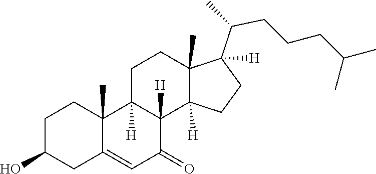 |
| 15 | 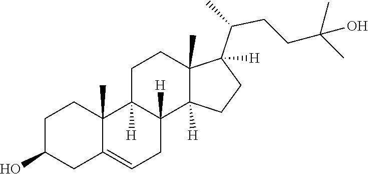 |
| 16 | 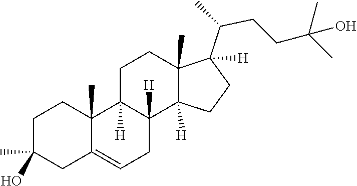 |
| 17 | 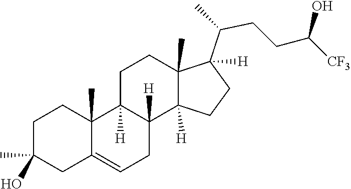 |
| 18 | 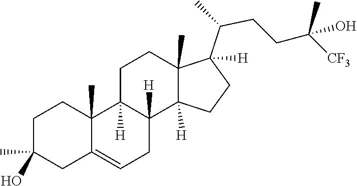 |
| 19 | 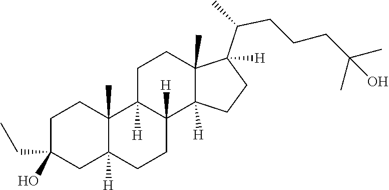 |
| 20 | 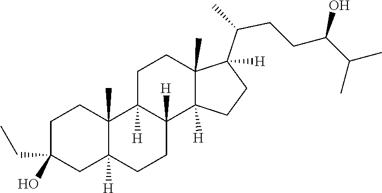 |
| 21 | 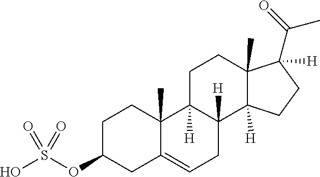 |
| 22 | 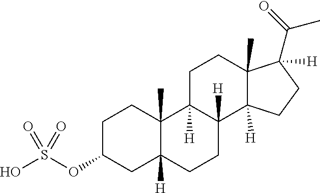 |
| 23 | 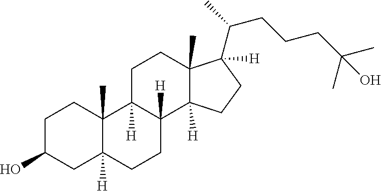 |
| 24 | 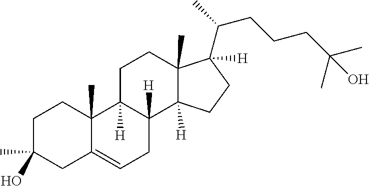 |
| 25 | 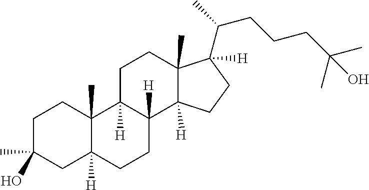 |
| 26 | 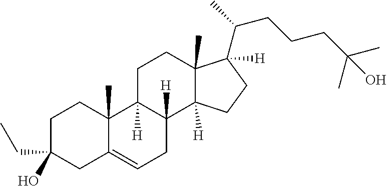 |
| 27 | 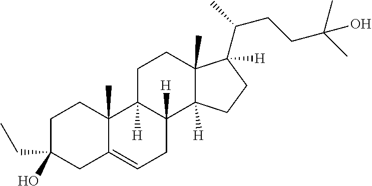 |

and deuterated variants thereof, wherein the point(s) of attachment of Agent to Z replaces one or more hydrogen atoms on the Agent.

In some embodiments, W is

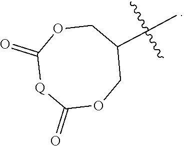

In some embodiments, W is

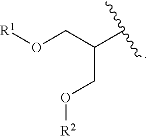

In some embodiments, W is

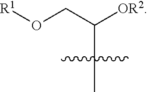

In some embodiments, W is

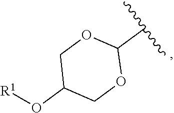

In some embodiments, W is —C(O)N—(R³)₂.

In some embodiments, Q is —C₁₋₈ alkylene-. In some embodiments, Q is —C₁₋₆ alkylene-. In some embodiments, Q is —C₁ alkylene-. In some embodiments, Q is —C₂ alkylene-. In some embodiments, Q is —C₃ alkylene-. In some embodiments, Q is —C₄ alkylene-. In some embodiments, Q is —C₅ alkylene-. In some embodiments, Q is —C₆ alkylene-. In some embodiments, Q is —C₇ alkylene-. In some embodiments, Q is —C₈ alkylene-.

In some embodiments, R¹ is hydrogen. In some embodiments, R¹ is an acid-labile group. In some embodiments, R¹ is a lipid. In some embodiments, R¹ is a fatty acid. In some embodiments, R¹ is —C(O)R³.

In some embodiments, R² is hydrogen. In some embodiments, R² is an acid-labile group. In some embodiments, R² is a lipid. In some embodiments, R² is a fatty acid. In some embodiments, R² is —C(O)R³.

In some embodiments, R¹ is a lipid. In some embodiments, R² is a lipid. Examples of lipids include fatty acids. Exemplary fatty acids may be saturated or unsaturated medium-chain or long-chain fatty acids. In some embodiments, the fatty acid comprises a C₂₋₄₀ aliphatic group. In some embodiments, the fatty acid comprises a C₂ aliphatic group. In some embodiments, the fatty acid comprises a C₃ aliphatic group. In some embodiments, the fatty acid comprises a C₄ aliphatic group. In some embodiments, the fatty acid comprises a C₅ aliphatic group. In some embodiments, the fatty acid comprises a C₆ aliphatic group. In some embodiments, the fatty acid comprises a C₇ aliphatic group. In some embodiments, the fatty acid comprises a C₈ aliphatic group. In some embodiments, the fatty acid comprises a C₉ aliphatic group. In some embodiments, the fatty acid comprises a C₁₀ aliphatic group. In some embodiments, the fatty acid comprises a C₁₁ aliphatic group. In some embodiments, the fatty acid comprises a C₁₂ aliphatic group. In some embodiments, the fatty acid comprises a C₁₃ aliphatic group. In some embodiments, the fatty acid comprises a C₁₄ aliphatic group. In some embodiments, the fatty acid comprises a C₁₅ aliphatic group. In some embodiments, the fatty acid comprises a C₁₆ aliphatic group. In some embodiments, the fatty acid comprises a C₁₇ aliphatic group. In some embodiments, the fatty acid comprises a C₁₈ aliphatic group. In some embodiments, the fatty acid comprises a C₁₉ aliphatic group. In some embodiments, the fatty acid comprises a C₂₀ aliphatic group. In some embodiments, the fatty acid comprises a C₂₁ aliphatic group. In some embodiments, the fatty acid comprises a C₂₂ aliphatic group. In some embodiments, the fatty acid comprises a C₂₃ aliphatic group. In some embodiments, the fatty acid comprises a C₂₄ aliphatic group. In some embodiments, the fatty acid comprises a C₂₅ aliphatic group. In some embodiments, the fatty acid comprises a C₂₆ aliphatic group. In some embodiments, the fatty acid comprises a C₂₇ aliphatic group. In some embodiments, the fatty acid comprises a C₂₈ aliphatic group. In some embodiments, the fatty acid comprises a C₂₉ aliphatic group. In some embodiments, the fatty acid comprises a C₃₀ aliphatic group. In some embodiments, the fatty acid comprises a C₃₁ aliphatic group. In some embodiments, the fatty acid comprises a C₃₂ aliphatic group. In some embodiments, the fatty acid comprises a C₃₃ aliphatic group. In some embodiments, the fatty acid comprises a C₃₄ aliphatic group. In some embodiments, the fatty acid comprises a C₃₅ aliphatic group. In some embodiments, the fatty acid comprises a C₃₆ aliphatic group. In some embodiments, the fatty acid comprises a C₃₇ aliphatic group. In some embodiments, the fatty acid comprises a C₃₈ aliphatic group. In some embodiments, the fatty acid comprises a C₃₉ aliphatic group. In some embodiments, the fatty acid comprises a C₄₀ aliphatic group.

In some embodiments, R¹ is an acid labile group. In some embodiments, R² is an acid labile group. Exemplary acid labile groups include, but are not limited to, —C(O)OR, —C(O)NR², —CH₂OR, —C(NR)R, —P(O)₂OR, an amino acid or a PEG group.

In some embodiments, R¹ and R² are the same. In some embodiments, R¹ and R² are different.

In some embodiments, each R³ is independently a substituted C₁₋₄₀ aliphatic. In some embodiments, each R³ is independently a substituted C₁ aliphatic. In some embodiments, each R³ is independently a substituted C₂ aliphatic. In some embodiments, each R³ is independently a substituted C₃ aliphatic. In some embodiments, each R³ is independently a substituted C₄ aliphatic. In some embodiments, each R³ is independently a substituted C₅ aliphatic. In some embodiments, each R³ is independently a substituted C₆ aliphatic. In some embodiments, each R³ is independently a substituted C₇ aliphatic. In some embodiments, each R³ is independently a substituted C₈ aliphatic. In some embodiments, each R³ is independently a substituted C₉ aliphatic. In some embodiments, each R³ is independently a substituted C₁₀ aliphatic. In some embodiments, each R³ is independently a substituted C₁₁ aliphatic. In some embodiments, each R³ is independently a substituted C₁₂ aliphatic. In some embodiments, each R³ is independently a substituted C₁₃ aliphatic. In some embodiments, each R³ is independently a substituted C₁₄ aliphatic. In some embodiments, each R³ is independently a substituted C₁₅ aliphatic. In some embodiments, each R³ is independently a substituted C₁₆ aliphatic. In some embodiments, each R³ is independently a substituted C₁₇ aliphatic. In some embodiments, each R³ is independently a substituted C₁₈ aliphatic. In some embodiments, each R³ is independently a substituted C₁₉ aliphatic. In some embodiments, each R³ is independently a substituted C₂₀ aliphatic. In some embodiments, each R³ is independently a substituted C₂₁ aliphatic. In some embodiments, each R³ is independently a substituted C₂₂ aliphatic. In some embodiments, each R³ is independently a substituted C₂₃ aliphatic. In some embodiments, each R³ is independently a substituted C₂₄ aliphatic. In some embodiments, each R³ is independently a substituted C₂₅ aliphatic. In some embodiments, each R³ is independently a substituted C₂₆ aliphatic. In some embodiments, each R³ is independently a substituted C₂₇ aliphatic. In some embodiments, each R³ is independently a substituted C₂₈ aliphatic. In some embodiments, each R³ is independently a substituted C₂₉ aliphatic. In some embodiments, each R³ is independently a substituted C₃₀ aliphatic. In some embodiments, each R³ is independently a substituted C₃₁ aliphatic. In some embodiments, each R³ is independently a substituted C₃₂ aliphatic. In some embodiments, each R³ is independently a substituted C₃₃ aliphatic. In some embodiments, each R³ is independently a substituted C₃₄ aliphatic. In some embodiments, each R³ is independently a substituted C₃₅ aliphatic. In some embodiments, each R³ is independently a substituted C₃₆ aliphatic. In some embodiments, each R³ is independently a substituted C₃₇ aliphatic. In some embodiments, each R³ is independently a substituted C₃₈ aliphatic. In some embodiments, each R³ is independently a substituted C₃₉ aliphatic. In some embodiments, each R³ is independently a substituted C₄₀ aliphatic.

In some embodiments, each R³ is independently an optionally substituted C₁₋₄₀ aliphatic. In some embodiments, each R³ is independently an optionally substituted C₁ aliphatic. In some embodiments, each R³ is independently an optionally substituted C₂ aliphatic. In some embodiments, each R³ is independently an optionally substituted C₃ aliphatic. In some embodiments, each R³ is independently an optionally substituted C₄ aliphatic. In some embodiments, each R³ is independently an optionally substituted C₅ aliphatic. In some embodiments, each R³ is independently an optionally substituted C₆ aliphatic. In some embodiments, each R³ is independently an optionally substituted C₇ aliphatic. In some embodiments, each R³ is independently an optionally substituted C₈ aliphatic. In some embodiments, each R³ is independently an optionally substituted C₉ aliphatic. In some embodiments, each R³ is independently an optionally substituted C₁₀ aliphatic. In some embodiments, each R³ is independently an optionally substituted C₁₁ aliphatic. In some embodiments, each R³ is independently an optionally substituted C₁₂ aliphatic. In some embodiments, each R³ is independently an optionally substituted C₁₃ aliphatic. In some embodiments, each R³ is independently an optionally substituted C₁₄ aliphatic. In some embodiments, each R³ is independently an optionally substituted C₁₅ aliphatic. In some embodiments, each R³ is independently an optionally substituted C₁₆ aliphatic. In some embodiments, each R³ is independently an optionally substituted C₁₇ aliphatic. In some embodiments, each R³ is independently an optionally substituted C₁₈ aliphatic. In some embodiments, each R³ is independently an optionally substituted C₁₉ aliphatic. In some embodiments, each R³ is independently an optionally substituted C₂₀ aliphatic. In some embodiments, each R³ is independently an optionally substituted C₂₁ aliphatic. In some embodiments, each R³ is independently an optionally substituted C₂₂ aliphatic. In some embodiments, each R³ is independently an optionally substituted C₂₃ aliphatic. In some embodiments, each R³ is independently an optionally substituted C₂₄ aliphatic. In some embodiments, each R³ is independently an optionally substituted C₂₅ aliphatic. In some embodiments, each R³ is independently an optionally substituted C₂₆ aliphatic. In some embodiments, each R³ is independently an optionally substituted C₂₇ aliphatic. In some embodiments, each R³ is independently an optionally substituted C₂₈ aliphatic. In some embodiments, each R³ is independently an optionally substituted C₂₉ aliphatic. In some embodiments, each R³ is independently an optionally substituted C₃₀ aliphatic. In some embodiments, each R³ is independently an optionally substituted C₃₁ aliphatic. In some embodiments, each R³ is independently an optionally substituted C₃₂ aliphatic. In some embodiments, each R³ is independently an optionally substituted C₃₃ aliphatic. In some embodiments, each R³ is independently an optionally substituted C₃₄ aliphatic. In some embodiments, each R³ is independently an optionally substituted C₃₅ aliphatic. In some embodiments, each R³ is independently an optionally substituted C₃₆ aliphatic. In some embodiments, each R³ is independently an optionally substituted C₃₇ aliphatic. In some embodiments, each R³ is independently an optionally substituted C₃₈ aliphatic. In some embodiments, each R³ is independently an optionally substituted C₃₉ aliphatic. In some embodiments, each R³ is independently an optionally substituted C₄₀ aliphatic.

In some embodiments, each R³ is independently C₁₋₄₀ aliphatic. In some embodiments, each R³ is independently C₁ aliphatic. In some embodiments, each R³ is independently C₂ aliphatic. In some embodiments, each R³ is independently C₃ aliphatic. In some embodiments, each R³ is independently C₄ aliphatic. In some embodiments, each R³ is independently C₅ aliphatic. In some embodiments, each R³ is independently C₆ aliphatic. In some embodiments, each R³ is independently C₇ aliphatic. In some embodiments, each R³ is independently C₈ aliphatic. In some embodiments, each R³ is independently C₉ aliphatic. In some embodiments, each R³ is independently C₁₀ aliphatic. In some embodiments, each R³ is independently C₁₁ aliphatic. In some embodiments, each R³ is independently C₁₂ aliphatic. In some embodiments, each R³ is independently C₁₃ aliphatic. In some embodiments, each R³ is independently C₁₄ aliphatic. In some embodiments, each R³ is independently C₁₅ aliphatic. In some embodiments, each R³ is independently C₁₆ aliphatic. In some embodiments, each R³ is independently C₁₇ aliphatic. In some embodiments, each R³ is independently C₁ₛ aliphatic. In some embodiments, each R³ is independently C₁₉ aliphatic. In some embodiments, each R³ is independently C₂₀ aliphatic. In some embodiments, each R³ is independently C₂₁ aliphatic. In some embodiments, each R³ is independently C₂₂ aliphatic. In some embodiments, each R³ is independently C₂₃ aliphatic. In some embodiments, each R³ is independently C₂₄ aliphatic. In some embodiments, each R³ is independently C₂₅ aliphatic. In some embodiments, each R³ is independently C₂₆ aliphatic. In some embodiments, each R³ is independently C₂₇ aliphatic. In some embodiments, each R³ is independently C₂₈ aliphatic. In some embodiments, each R³ is independently C₂₉ aliphatic. In some embodiments, each R³ is independently C₃₀ aliphatic. In some embodiments, each R³ is independently C₃₁ aliphatic. In some embodiments, each R³ is independently C₃₂ aliphatic. In some embodiments, each R³ is independently C₃₃ aliphatic. In some embodiments, each R³ is independently C₃₄ aliphatic. In some embodiments, each R³ is independently C₃₅ aliphatic. In some embodiments, each R³ is independently C₃₆ aliphatic. In some embodiments, each R³ is independently C₃₇ aliphatic. In some embodiments, each R³ is independently C₃₈ aliphatic. In some embodiments, each R³ is independently C₃₉ aliphatic. In some embodiments, each R³ is independently C₄₀ aliphatic.

In some embodiments, X is —O—. In some embodiments, X is —NR—. In some embodiments, X is —S—. In some embodiments, X is —O(C₁₋₆ aliphatic)-O—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —O(C₁₋₆ aliphatic)-S—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —O(C₁₋₆ aliphatic)-NR—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —S(C₁₋₆ aliphatic)-O—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —S(C₁₋₆ aliphatic)-S—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —S(C₁₋₆ aliphatic)-NR—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —NR(C₁₋₆ aliphatic)-O—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —NR(C₁₋₆ aliphatic)-S—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —NR(C₁₋₆ aliphatic)-NR—, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen. In some embodiments, X is —(C₁₋₆ aliphatic)-. In some embodiments, X is —O(C₁₋₆ aliphatic)-O—. In some embodiments, X is —O(C₁₋₆ aliphatic)-S—. In some embodiments, X is —O(C₁₋₆ aliphatic)-NR—. In some embodiments, X is —S(C₁₋₆ aliphatic)-O—. In some embodiments, X is —S(C₁₋₆ aliphatic)-S—. In some embodiments, X is —S(C₁₋₆ aliphatic)-NR—. In some embodiments, X is —NR(C₁₋₆ aliphatic)-O—. In some embodiments, X is —NR(C₁₋₆ aliphatic)-S—. In some embodiments, X is —NR(C₁₋₆ aliphatic)-NR—. In some embodiments, X is —(C₁₋₆ aliphatic)-.

In some embodiments, R is hydrogen.

In some embodiments, R is an optionally substituted ₋₆ aliphatic. In some embodiments, R is an optionally substituted 3-8 membered carbocycle. In some embodiments, R is an optionally substituted C₆₋₁₀ aryl. In some embodiments, R is an optionally substituted 4-8 membered heterocycle comprising 1-2 heteroatoms independently selected from the group consisting of N, O and S. In some embodiments, R is an optionally substituted 5-10 membered heteroaryl comprising 1-4 heteroatoms independently selected from the group consisting of N, O, and S.

In some embodiments, R is selected from the group consisting of hydrogen, C₁₋₆ aliphatic, 3-8 membered carbocycle, C₆₋₁₀ aryl, 4-8 membered heterocycle comprising 1-2 heteroatoms independently selected from the group consisting of N, O and S, and 5-10 membered heteroaryl comprising 1-4 heteroatoms. In some embodiments, R is a C₁₋₆ aliphatic. In some embodiments, R is a 3-8 membered carbocycle. In some embodiments, R is a 4-8 membered heterocycle comprising 1-2 heteroatoms independently selected from the group consisting of N, O and S. In some embodiments, R is a 5-10 membered heteroaryl comprising 1-4 heteroatoms independently selected from the group consisting of N, O and S.

In some embodiments, R is a substituted ₋₆ aliphatic. In some embodiments, R is a substituted 3-8 membered carbocycle. In some embodiments, R is a substituted C₆₋₁₀aryl. In some embodiments, R is a substituted 4-8 membered heterocycle comprising 1-2 heteroatoms independently selected from the group consisting of N, O and S. In some embodiments, R is a substituted 5-10 membered heteroaryl comprising 1-4 heteroatoms independently selected from the group consisting of N, O and S.

In some embodiments, Y is absent. In some embodiments, Y is —C(O)—. In some embodiments, Y is —C(NR)—. In some embodiments, Y is —C(S)—.

In some embodiments, Z is absent. In some embodiments, Z is an optionally substituted bivalent C₁₋₃₀ aliphatic, wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-. In some embodiments, Z is an optionally substituted C₁₋₃₀ alkylene (i.e., C₁alkylene, C₂alkylene, C₃alkylene, C₄alkylene, C₅alkylene, C₆alkylene, C₇alkylene, C₈alkylene, C₉alkylene, C₁₀alkylene, C₁₁alkylene, C₁₂alkylene, C₁₃alkylene, C₁₄alkylene, C₁₅alkylene, C₁₆alkylene, C₁₇alkylene, C₁₈alkylene, C₁₉alkylene, C₂₀alkylene, C₂₁alkylene, C₂₂alkylene, C₂₃alkylene, C₂₄alkylene, C₂₅alkylene, C₂₆alkylene, C₂₇alkylene, C₂₈alkylene, C₂₉alkylene or C₃₀alkylene), wherein 0-8 (i.e., 0, 1, 2, 3, 4, 5, 6, 7, or 8) methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-.

In some embodiments, Z is an optionally substituted C₂₋₃₀alkenylene (i.e., C₂alkenylene, C₃alkenylene, C₄alkenylene, C₅alkenylene, C₆alkenylene, C₇alkenylene, C₈alkenylene, C₉alkenylene, C₁₀alkenylene, C₁₁alkenylene, C₁₂alkenylene, C₁₃alkenylene, C₁₄alkenylene, C₁₅alkenylene, C₁₆alkenylene, C₁₇alkenylene, C₁₈alkenylene, C₁₉alkenylene, C₂₀alkenylene, C₂₁alkenylene, C₂₂alkenylene, C₂₃alkenylene, C₂₄alkenylene, C₂₅alkenylene, C₂₆alkenylene, C₂₇alkenylene, C₂₈alkenylene, C₂₉alkenylene or C₃₀alkenylene), wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-.

In some embodiments, Z is an optionally substituted C₂₋₃₀alkynylene (i.e., C₂alkynylene, C₃alkynylene, C₄alkynylene, C₅alkynylene, C₆alkynylene, C₇alkynylene, C₈alkynylene, C₉alkynylene, C₁₀alkynylene, C₁₁alkynylene, C₁₂alkynylene, C₁₃alkynylene, C₁₄alkynylene, C₁₅alkynylene, C₁₆alkynylene, C₁₇alkynylene, C₁₈alkynylene, C₁₉alkynylene, C₂₀alkynylene, C₂₁alkynylene, C₂₂alkynylene, C₂₃alkynylene, C₂₄alkynylene, C₂₅alkynylene, C₂₆alkynylene, C₂₇alkynylene, C₂₈alkynylene, C₂₉alkynylene or C₃₀alkynylene), wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-.

In some embodiments, Z is a bivalent C₁₋₃₀ aliphatic, wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-. In some embodiments, Z is C₁₋₃₀ alkylene (i.e., C₁alkylene, C₂alkylene, C₃alkylene, C₄alkylene, C₅alkylene, C₆alkylene, C₇alkylene, C₈alkylene, C₉alkylene, C₁₀alkylene, C₁₁alkylene, C₁₂alkylene, C₁₃alkylene, C₁₄alkylene, C₁₅alkylene, C₁₆alkylene, C₁₇alkylene, C₁₈alkylene, C₁₉alkylene, C₂₀alkylene, C₂₁alkylene, C₂₂alkylene, C₂₃alkylene, C₂₄alkylene, C₂₅alkylene, C₂₆alkylene, C₂₇alkylene, C₂₈alkylene, C₂₉alkylene or C₃₀alkylene), wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-.

In some embodiments, Z is C₂₋₃₀alkenylene (i.e., C₂alkenylene, C₃alkenylene, C₄alkenylene, C₅alkenylene, C₆alkenylene, C₇alkenylene, C₈alkenylene, C₉alkenylene, C₁₀alkenylene, C₁₁alkenylene, C₁₂alkenylene, C₁₃alkenylene, C₁₄alkenylene, C₁₅alkenylene, C₁₆alkenylene, C₁₇alkenylene, C₁₈alkenylene, C₁₉alkenylene, C₂₀alkenylene, C₂₁alkenylene, C₂₂alkenylene, C₂₃alkenylene, C₂₄alkenylene, C₂₅alkenylene, C₂₆alkenylene, C₂₇alkenylene, C₂₈alkenylene, C₂₉alkenylene or C₃₀alkenylene), wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-.

In some embodiments, Z is C₂₋₃₀alkynylene (i.e., C₂alkynylene, C₃alkynylene, C₄alkynylene, C₅alkynylene, C₆alkynylene, C₇alkynylene, C₈alkynylene, C₉alkynylene, C₁₀alkynylene, C₁₁alkynylene, C₁₂alkynylene, C₁₃alkynylene, C₁₄alkynylene, C₁₅alkynylene, C₁₆alkynylene, C₁₇alkynylene, C₁₈alkynylene, C₁₉alkynylene, C₂₀alkynylene, C₂₁alkynylene, C₂₂alkynylene, C₂₃alkynylene, C₂₄alkynylene, C₂₅alkynylene, C₂₆alkynylene, C₂₇alkynylene, C₂₈alkynylene, C₂₉alkynylene or C₃₀alkynylene), wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-.

In some embodiments, Z is selected from the group consisting of —C(R^{4a})(R^{4b})C(O)-M-, —C(R^{4a})(R^{4b})-M-, —C(R^{4a})(R^{4b})C(R^{5a})(R^{5b})(CH₂)ₙC(O)-M-, —C(R^{4a})(R^{4b})C(R^{5a})(R^{5b})(CH₂)ₙ-M-, —(CH₂)ₘC(R^{4a})(R^{4b})(CH₂)ₙC(R^{5a})(R^{5b})(CH₂)ₘC(O)-M-, and —(CH₂)ₘC(R^{4a})(R^{4b})(CH₂)ₙC(R^{5a})(R^{5b})(CH₂)ₘ-M-, wherein either side of Z may be attached to Agent.

In some embodiments, Z is selected from the group consisting of —CH(R^{4a})C(O)-M-, —CH(R^{4a})-M-, —CH(R^{4a})CH(R^{5a})C(O)-M-, —CH(R^{4a})CH(R^{5a})(CH₂)ₙC(O)-M-, —CH(R^{4a})CH(R^{5a})(CH₂)ₙ-M-, and —C(R^{4a})(R^{4b})(CH₂)ₙC(R^{5a})(R^{5b})C(O)-M-, wherein either side of Z may be attached to Agent.

In some embodiments, 0-8 methylene units of Z are independently replaced by —R⁸—. In some embodiments, 0-8 methylene units of Z are independently replaced by —O—. In some embodiments, 0-8 methylene units of Z are independently replaced by —NR—. In some embodiments, 0-8 methylene units of Z are independently replaced by —S—. In some embodiments, 0-8 methylene units of Z are independently replaced by —OC(O)—. In some embodiments, 0-8 methylene units of Z are independently replaced by —C(O)O—. In some embodiments, 0-8 methylene units of Z are independently replaced by —C(O)—. In some embodiments, 0-8 methylene units of Z are independently replaced by —S(O)—. In some embodiments, 0-8 methylene units of Z are independently replaced by —S(O)₂—. In some embodiments, 0-8 methylene units of Z are independently replaced by —C(S)—. In some embodiments, 0-8 methylene units of Z are independently replaced by —OS(O)₂—. In some embodiments, 0-8 methylene units of Z are independently replaced by —S(O)₂O—. In some embodiments, 0-8 methylene units of Z are independently replaced by —N(R)S(O)₂—. In some embodiments, 0-8 methylene units of Z are independently replaced by —S(O)₂NR—. In some embodiments, 0-8 methylene units of Z are independently replaced by —N(R)C(O)—. In some embodiments, 0-8 methylene units of Z are independently replaced by —C(O)NR—. In some embodiments, 0-8 methylene units of Z are independently replaced by —OC(O)NR—. In some embodiments, 0-8 methylene units of Z are independently replaced by —N(R)C(O)O—. In some embodiments, 0-8 methylene units of Z are independently replaced by an amino acid.

In some embodiments, the amino acid may be naturally-occurring or non-naturally occurring. In some embodiments, the amino acid is an L-amino acid. In some embodiments, the amino acid is a D-amino acid. Amino acids include, but are not limited to glycine, alanine, leucine, isoleucine, valine, tyrosine, lysine, serine, threonine, methionine, proline, phenylalanine, tryptophan, asparagine, glutamine, aspartic acid, glutamic acid, arginine, histidine, cysteine, and selenocysteine. In some embodiments, the amino acid is selected from the group consisting of

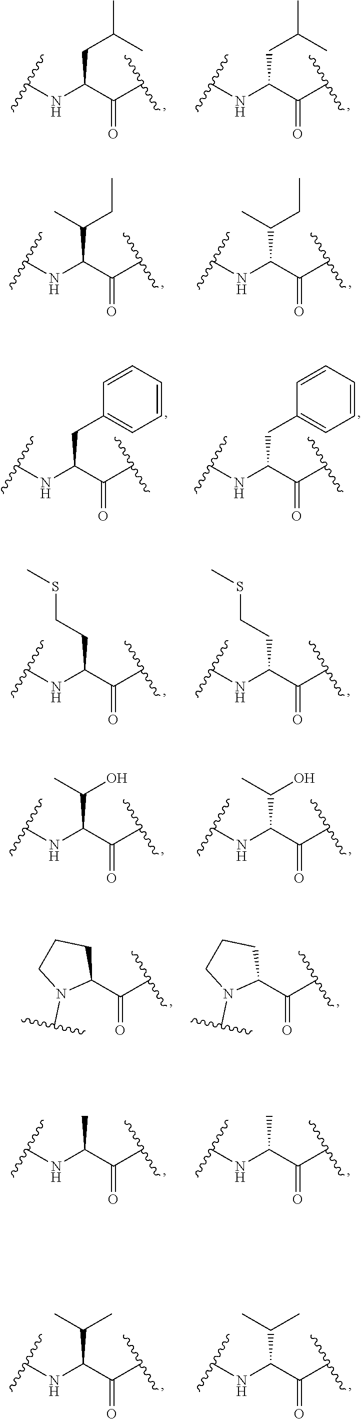

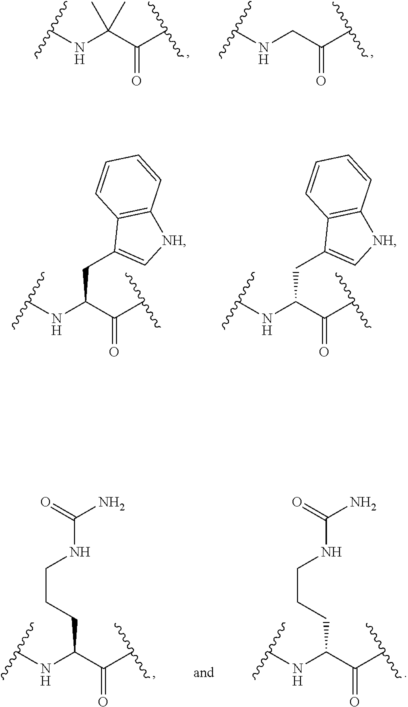

In some embodiments, Z is a bivalent C₁₋₂₅, C₅₋₂₅, C₇₋₂₅, or C₁₋₂₀ aliphatic optionally substituted with 1, 2, 3, or 4 groups selected from deuterium, halogen, —CN, a 3-8 membered carbocycle, C₆₋₁₀aryl, a 4-8 membered heterocycle comprising 1-2 heteroatoms independently selected from the group consisting of N, O and S, a 5-10 membered heteroaryl comprising 1-4 heteroatoms independently selected from the group consisting of N, O, and S, or a C₁₋₆ aliphatic group optionally substituted with 1, 2, 3, 4, 5, or 6 deuterium or halogen atoms; wherein 0-4 methylene units of Z are independently replaced by —O—, —OC(O)—, —C(O)O—, or —C(O)—; and 1 methylene unit of Z is optionally replaced with -M-.

In some embodiments, each —R⁸— is independently an optionally substituted bivalent C₃₋₆ carbocycle. In some embodiments, each —R⁸— is independently an optionally substituted bivalent C₆₋₁₀ aryl. In some embodiments, each —R⁸— is independently an optionally substituted bivalent 3-6 membered heterocycle comprising 1-4 heteroatoms independently selected from the group consisting of N, O, and S. In some embodiments, each —R⁸— is independently an optionally substituted bivalent 5-10 membered heteroaryl comprising 1-4 heteroatoms independently selected from the group consisting of N, O, and S.

In some embodiments, each —R⁸— is independently a bivalent group selected from the group consisting of a C₃₋₆ carbocycle, a C₆₋₁₀ aryl, a 3-6 membered heterocycle comprising 1-4 heteroatoms independently selected from the group consisting of nitrogen, oxygen, and sulfur; and 5-10 membered heteroaryl comprising 1-4 heteroatoms independently selected from the group consisting of nitrogen, oxygen, and sulfur. In some embodiments, each —R⁸— is independently a bivalent C₃₋₆ carbocycle. In some embodiments, each —R⁸— is independently a bivalent C₆₋₁₀ aryl. In some embodiments, each —R⁸— is independently a bivalent substituted 3-6 membered heterocycle comprising 1-4 heteroatoms independently selected from the group consisting of N, O, and S. In some embodiments, each —R⁸— is independently a bivalent substituted 5-10 membered heteroaryl comprising 1-4 heteroatoms independently selected from the group consisting of N, O, and S.

In some embodiments, R^{4a} is selected from the group consisting of hydrogen, deuterium, halogen, —CN, —OR, —NR₂, —SR, —R⁹, and —R¹⁰. In some embodiments, R^{4a} is hydrogen. In some embodiments, R^{4a} is deuterium. In some embodiments, R^{4a} is halogen. In some embodiments, R^{4a} is —CN. In some embodiments, R^{4a} is —OR. In some embodiments, R^{4a} is —NR₂. In some embodiments, R^{4a} is —SR. In some embodiments, R^{4a} is —R⁹. In some embodiments, R^{4a} is —R¹⁰.

In some embodiments, R^{4b} is independently selected from the group consisting of hydrogen, deuterium, halogen, —CN, —OR, —NR₂, —SR, —R⁹, and —R¹⁰. In some embodiments, R^{4b} is hydrogen. In some embodiments, R^{4b} is deuterium. In some embodiments, R^{4b} is halogen. In some embodiments, R^{4b} is —CN. In some embodiments, R^{4b} is —OR. In some embodiments, R^{4b} is —NR₂. In some embodiments, R^{4b} is —SR. In some embodiments, R^{4b} is —R⁹. In some embodiments, R^{4b} is —R¹⁰.

In some embodiments, each of R^{4a} and R^{4b} is independently hydrogen, deuterium, halogen, —CN, or C₁₄ aliphatic optionally substituted with 1-6 deuterium or halogen atoms; or R^{4a} and R^{4b} together with the carbon atom to which they are attached, form a C₃₋₆ carbocycle or a 3-6 membered heterocycle comprising 1-2 heteroatoms independently selected from the group consisting of N, O and S.

In some embodiments, R^{5a} is selected from the group consisting of hydrogen, deuterium, halogen, —CN, —OR, —NR₂, —SR, —R⁹, and —R¹⁰. In some embodiments, R^{5a} is hydrogen. In some embodiments, R^{5a} is deuterium. In some embodiments, R^{5a} is halogen. In some embodiments, R^{5a} is —CN. In some embodiments, R^{5a} is —OR. In some embodiments, R^{5a} is —NR₂. In some embodiments, R^{5a} is —SR. In some embodiments, R^{5a} is —R⁹. In some embodiments, R^{5a} is —R¹⁰.

In some embodiments, R^{5b} is independently selected from the group consisting of hydrogen, deuterium, halogen, —CN, —OR, —NR₂, —SR, —R⁹, and —R¹⁰. In some embodiments, R^{5b} is hydrogen. In some embodiments, R^{5b} is deuterium. In some embodiments, R^{5b} is halogen. In some embodiments, R^{5b} is —CN. In some embodiments, R^{5b} is —OR. In some embodiments, R^{5b} is —NR₂. In some embodiments, R^{5b} is —SR. In some embodiments, R^{5b} is —R⁹. In some embodiments, R^{5b} is —R¹⁰.

In some embodiments, each of R^{5a} and R^{5b} is independently hydrogen, deuterium, halogen, —CN, or C₁₄ aliphatic optionally substituted with 1-6 deuterium or halogen atoms; or R^{5a} and R^{5b} together with the carbon atom to which they are attached, form a C₃₋₆ carbocycle or a 3-6 membered heterocycle comprising 1-2 heteroatoms independently selected from the group consisting of N, O and S.

In some embodiments, each of R^{4a}, R^{4b}, R^{5a} and R^{5b} is independently hydrogen or C₁₋₄ alkyl optionally substituted with 1-6 deuterium or halogen atoms.

In some embodiments, at least one instance of R^{4a} and R^{4b} is not hydrogen.

In some embodiments, at least one instance of R^{5a} and R^{5b} is not hydrogen.

In some embodiments, R^{4a} and R^{4b} together with the carbon atom to which they are attached, form a C₃₋₆ carbocycle or a 3-6 membered heterocycle comprising 1-2 heteroatoms independently selected from the group consisting of N, O and S. In some embodiments, R^{4a} and R^{4b} together with the carbon atom to which they are attached, form a C₃₋₆ carbocycle. In some embodiments, R^{4a} and R^{4b} together with the carbon atom to which they are attached, form a 3-6 membered heterocycle comprising 1-2 heteroatoms independently selected from the group consisting of N, O and S.

In some embodiments, R^{5a} and R^{5b} together with the carbon atom to which they are attached, form a C₃₋₆ carbocycle or a 3-6 membered heterocycle comprising 1-2 heteroatoms independently selected from the group consisting of N, O and S. In some embodiments, R^{5a} and R^{5b} together with the carbon atom to which they are attached, form a C₃₋₆ carbocycle. In some embodiments, R^{5a} and R^{5b} together with the carbon atom to which they are attached, form a 3-6 membered heterocycle comprising 1-2 heteroatoms independently selected from the group consisting of N, O and S.

In some embodiments, R⁹ is a 3-8 membered carbocycle. In some embodiments, R⁹ is a C₆₋₁₀aryl. In some embodiments, R⁹ is a 4-8 membered heterocycle comprising 1-2 heteroatoms independently selected from the group consisting of N, O, and S. In some embodiments, R⁹ is a 5-10 membered heterocycle comprising 1-4 heteroatoms independently selected from the group consisting of N, O, and S.

In some embodiments, each of R¹⁰ is independently selected from the group consisting of C₁₋₆ aliphatic optionally substituted with —CN, —OR, —NR₂, —SR, or —R⁹, wherein said C₁₋₆ aliphatic is optionally and additionally substituted with 1-6 deuterium or halogen atoms. In some embodiments, R¹⁰ is an unsubstituted C₁₋₆ aliphatic. In some embodiments, R¹⁰ is C₁₋₆ aliphatic substituted with 1 to 6 —CN. In some embodiments, R¹⁰ is C₁₋₆ aliphatic substituted with 1 to 6 —OR. In some embodiments, R¹⁰ is C₁₋₆ aliphatic substituted with 1 to 6 —NR₂. In some embodiments, R¹⁰ is C₁₋₆ aliphatic substituted with 1 to 6 —SR. In some embodiments, R¹⁰ is C₁₋₆ aliphatic substituted with 1 to 6 —R⁹. In some embodiments, R¹⁰ is C₁₋₆ aliphatic substituted with 1 to 6 deuterium. In some embodiments, R¹⁰ is C₁₋₆ aliphatic substituted with 1 to 6 halogen.

In some embodiments, -M- is a cleavable moiety. In some embodiments, the cleavable moiety is a self-immolative moiety.

In some embodiments, -M- is selected from the group consisting of an acetal, an o-benzylalcohol, a p-benzylalcohol, a styryl group, a coumarin, and a group that self-immolates via a cyclization reaction. In some embodiments, -M- is selected from the group consisting of a disulfide, hydrazone, acetal self-immolative group, carboxyacetal self-immolative moiety, carboxy(methylacetal) self-immolative moiety, para-hydroxybenzyl carbonyl self-immolative moieties, flipped ester self-immolative moiety, trimethyl lock, or 2-hydroxyphenyl carbamate (2-HIPC) self-immolative moiety.

In some embodiments, -M- is selected from the group consisting of:

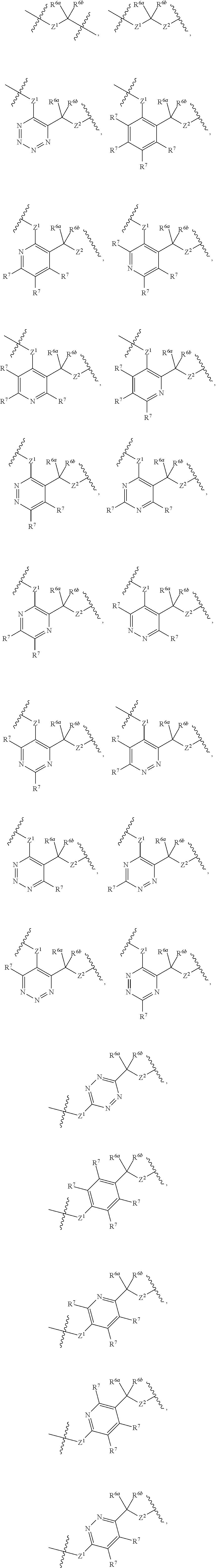

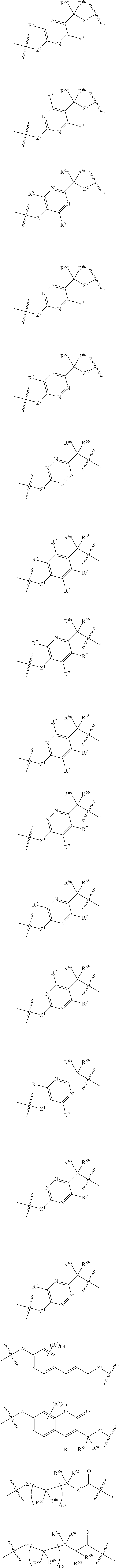

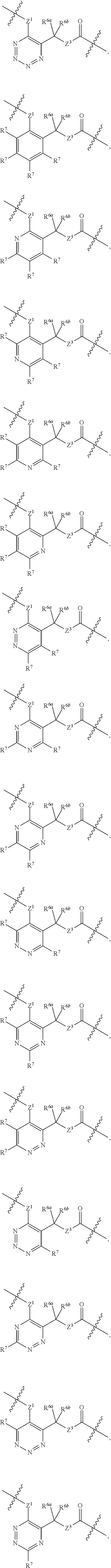

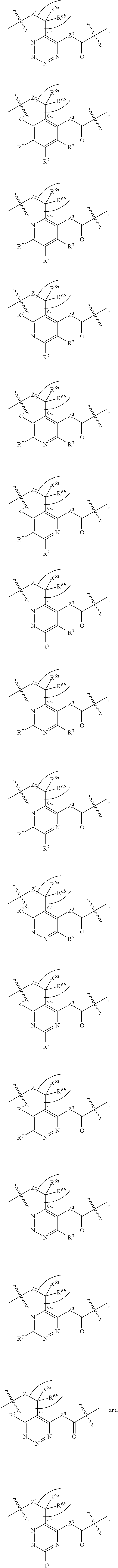

wherein either side of M may be attached to Agent; and
wherein each R^{6a} and R^{6b} is independently selected from the group consisting of hydrogen, deuterium, C₁₋₁₀ aliphatic, halogen, or —CN;
each R⁷ is independently selected from the group consisting of hydrogen, deuterium, halogen, —CN, —OR, —NR2, —NO₂, —SR, —R⁹, or —R¹⁰;
each Z¹ is independently selected from the group consisting —O—, —NR—, or —S—;
each Z² is independently selected from the group consisting —O—, —NR—, —S—, —OC(O)—, —N(R)C(O)O—, or —OC(O)NR—; and
each Z³ is independently selected from —O—, —NR—, —S—, —C(R^{6a})(R^{6b})—, or a covalent bond.

In some embodiments, R^{6a} is hydrogen. In some embodiments, R^{6a} is deuterium. In some embodiments, R^{6a} is C₁₋₆ aliphatic. In some embodiments, R^{6a} is C₁₋₆ alkyl. In some embodiments, R^{6a} is methyl, ethyl, propyl or isopropyl. In some embodiments, R^{6a} is methyl. In some embodiments, R^{6a} is ethyl. In some embodiments, R^{6a} is propyl. In some embodiments, R^{6a} is isopropyl. In some embodiments, R^{6a} is halogen. In some embodiments, R^{6a} is —CN.

In some embodiments, R^{6b} is hydrogen. In some embodiments, R^{6b} is deuterium. In some embodiments, R^{6b} is C₁₋₆ aliphatic. In some embodiments, R^{6b} is C₁₋₆ alkyl. In some embodiments, R^{6b} is methyl, ethyl, propyl or isopropyl. In some embodiments, R^{6b} is methyl. In some embodiments, R^{6b} is ethyl. In some embodiments, R^{6b} is propyl. In some embodiments, R^{6b} is isopropyl. In some embodiments, R^{6b} is halogen. In some embodiments, R^{6b} is —CN.

In some embodiments, each instance of R^{6a} is the same. In some embodiments, each instance of R^{6b} is the same. In some embodiments, each instance of R^{6a} and R^{6b} is the same. In some embodiments, each instance of R^{6a} is different. In some embodiments, each instance of R^{6b} is different. In some embodiments, each instance of R^{6a} and R^{6b} is different.

In some embodiments, each R⁷ is hydrogen. In some embodiments, each R⁷ is deuterium. In some embodiments, each R⁷ is halogen. In some embodiments, each R⁷ is —CN. In some embodiments, each R⁷ is —OR. In some embodiments, each R⁷ is —NR₂. In some embodiments, each R⁷ is —NO₂. In some embodiments, each R⁷ is —SR. In some embodiments, each R⁷ is —R⁹. In some embodiments, each R⁷ is —R¹⁰.

In some embodiments, Z¹ is —O—. In some embodiments, Z¹ is —NR—. In some embodiments, Z¹ is —S—. In some embodiments, Z¹ is —NH— or —NMe-.

In some embodiments, Z² is —O—. In some embodiments, Z² is —NR—. In some embodiments, Z² is —S—. In some embodiments, Z² is —OC(O)—. In some embodiments, Z² is —N(R)C(O)O—. In some embodiments, Z² is —OC(O)NR—. In some embodiments, Z² is —NH—. In some embodiments, Z² is —NMe-. In some embodiments, Z² is —NHC(O)O—. In some embodiments, Z² is —NMeC(O)O—. In some embodiments, Z² is —OC(O)NH—. In some embodiments, Z² is —OC(O)NMe-. In some embodiments, Z² is covalently bound to A.

In some embodiments, each Z³ is independently selected from —O—, —NR—, —S—, —C(R^{6a})(R^{6b})—, or a covalent bond. In some embodiments, Z⁴ is —O—. In some embodiments, Z³ is —NR—. In some embodiments, Z³ is —S—. In some embodiments, Z³ is —C(R^{6a})(R^{6b})—. In some embodiments, Z³ is a covalent bond.

In some embodiments, -M- is selected from the group consisting of:

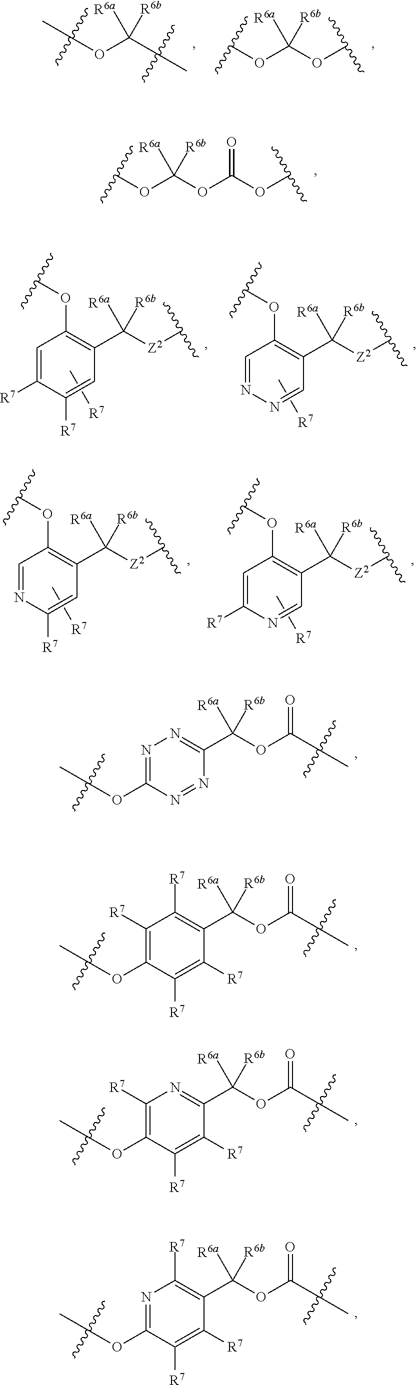

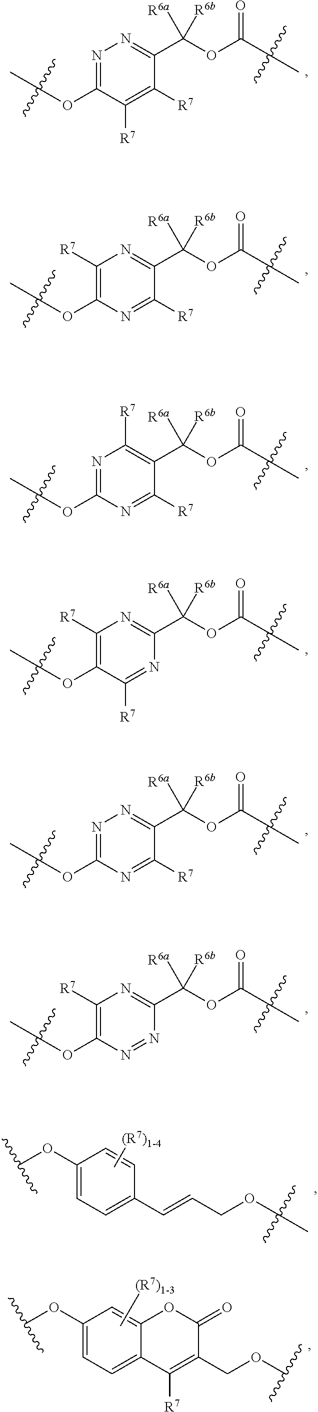

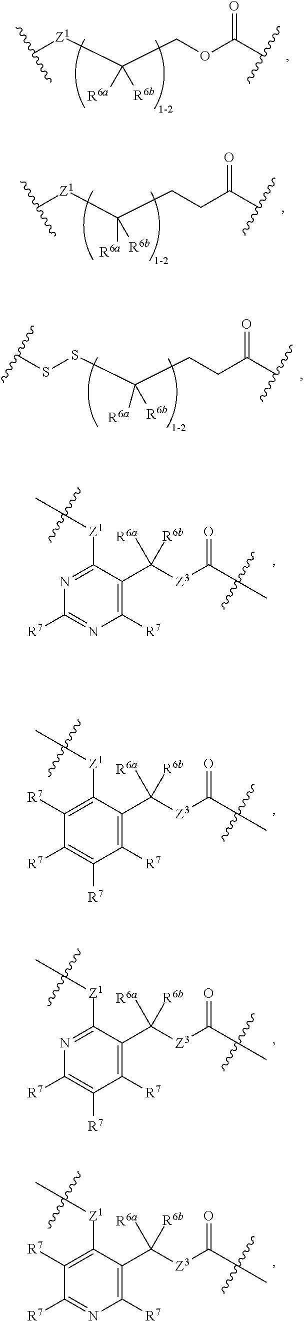

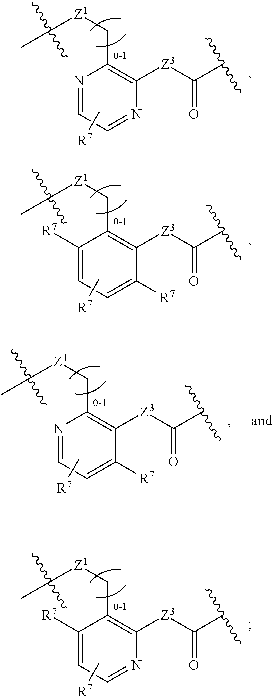

wherein either side of M may be attached to Agent.

In some embodiments, -M- is selected from the group consisting of:

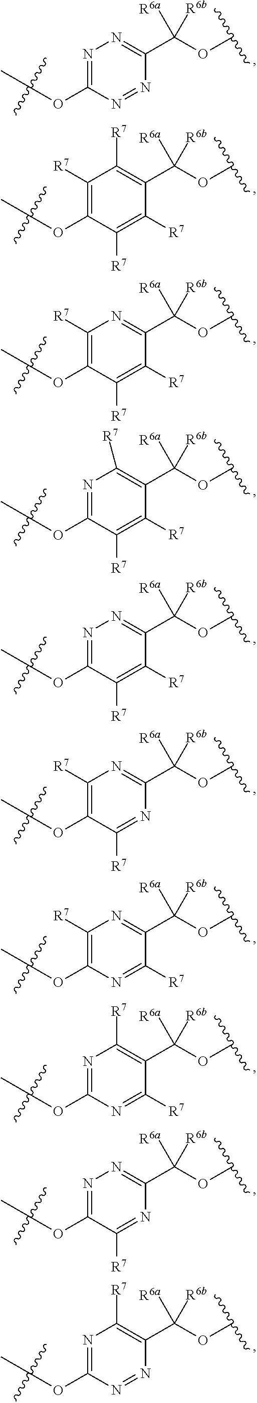

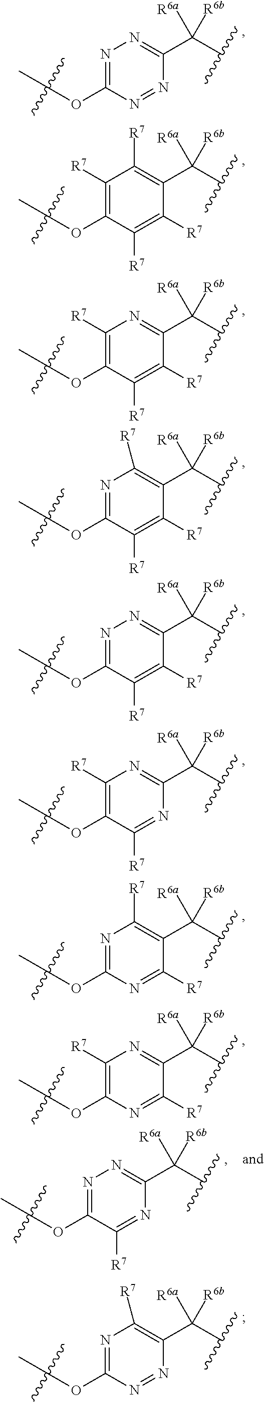

wherein either side of M may be attached to Agent.

In some embodiments, -M- is selected from the group consisting of:

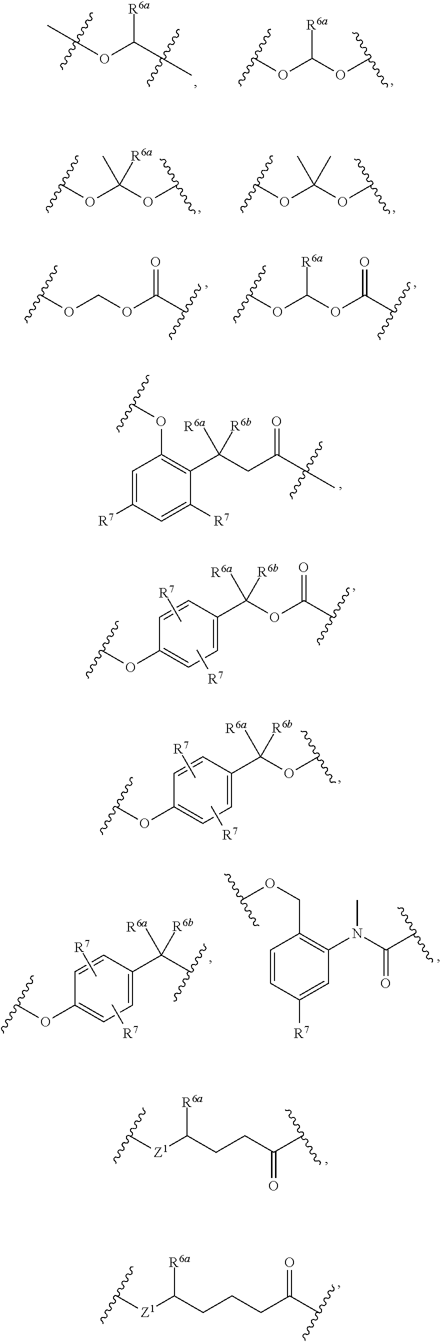

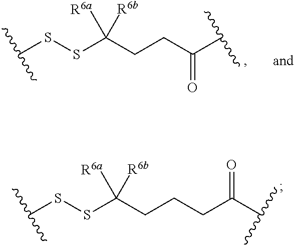

wherein either side of M may be attached to Agent.

In some embodiments, -M- is selected from the group consisting of:

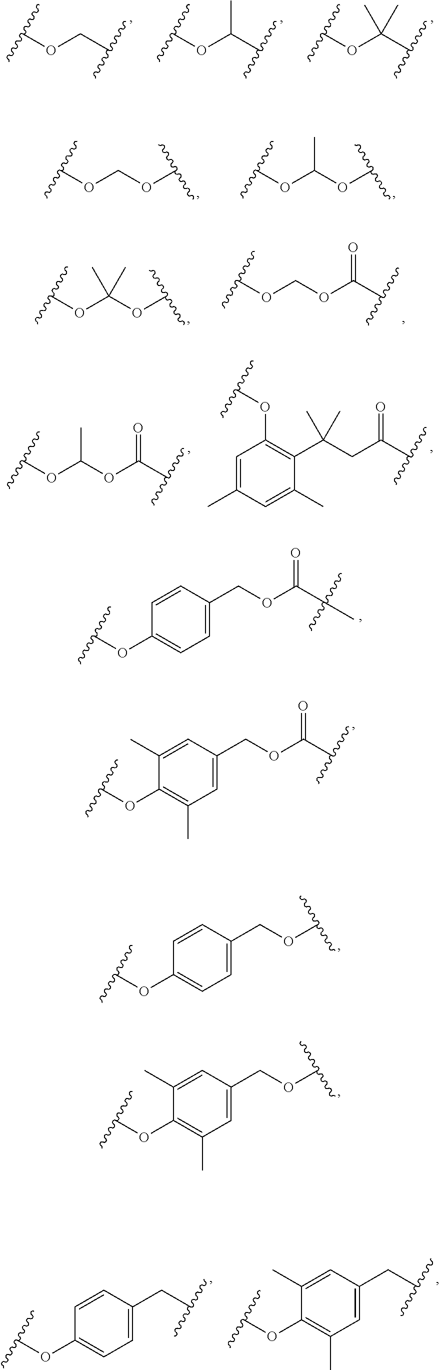

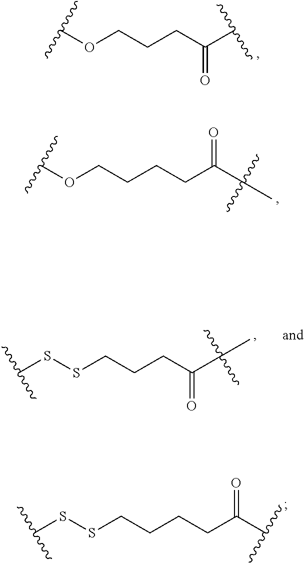 [SMILES: *OCCCC(*)=O ; *OCCCCC(*)=O ; *SSCCCC(*)=O ; *SSCCCCC(*)=O]

wherein either side of M may be attached to Agent.

In some embodiments, -M- is selected from the group consisting of:

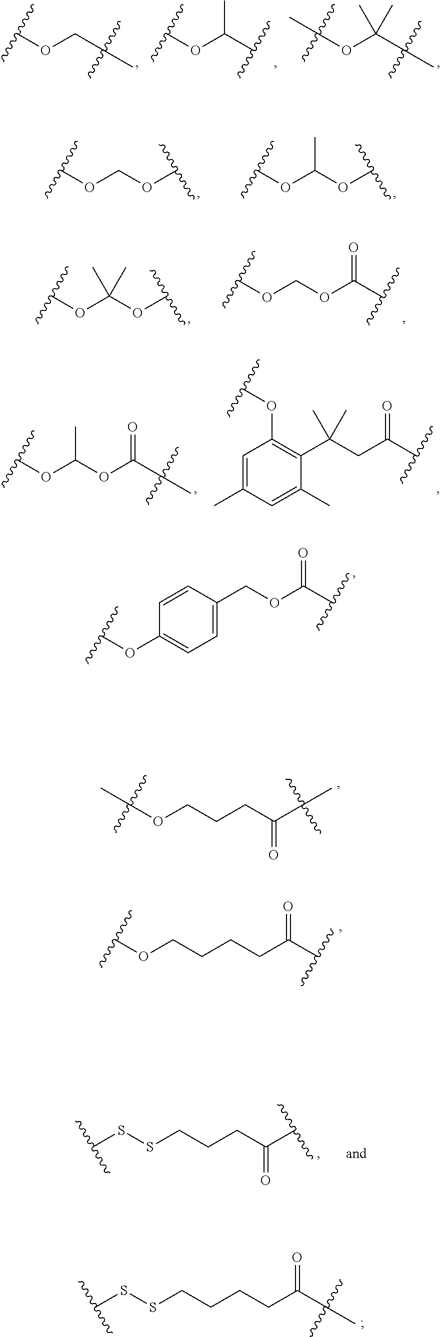

wherein either side of M may be attached to Agent.

In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, each m is independently 0, 1, or 2. In some embodiments, each m is independently 1, 2, 3, or 4.

In some embodiments, p is 1. In some embodiments, p is 2.

In some embodiments, Agent is any one of Agent Nos. 1-14, deuterated variants thereof, pharmaceutically acceptable salts thereof, or combinations thereof. In some embodiments, Agent is any one of Agent Nos. 15-20 or 23-27, deuterated variants thereof, pharmaceutically acceptable salts thereof, or combinations thereof. In some embodiments, Agent is any one of Agent Nos. 21 or 22, deuterated variants thereof, pharmaceutically acceptable salts thereof, or combinations thereof.

In some embodiments,

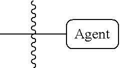

is a structure as set forth in Table 1, deuterated variants thereof, pharmaceutically acceptable salts thereof, or combinations thereof.

TABLE 1

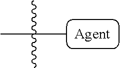

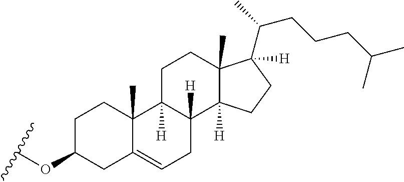

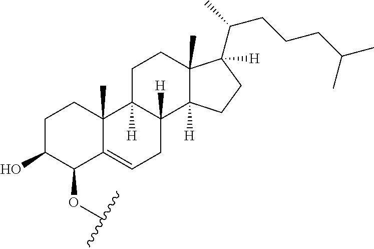

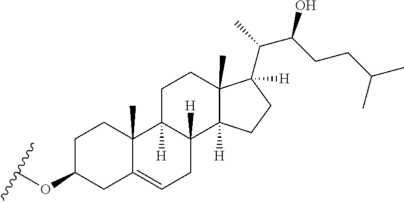

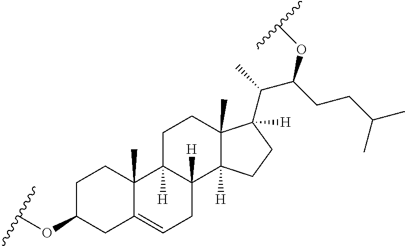

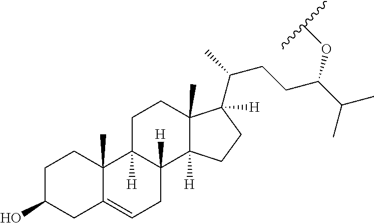

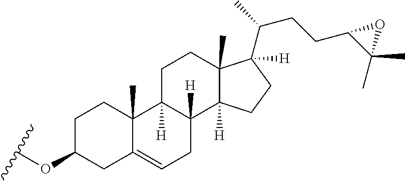

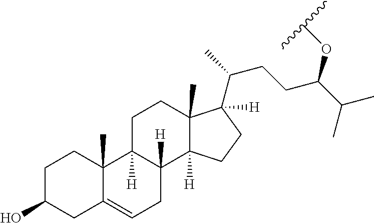

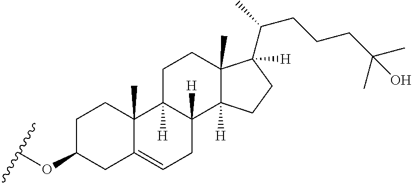

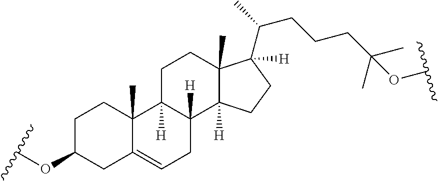

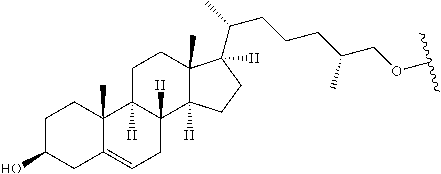

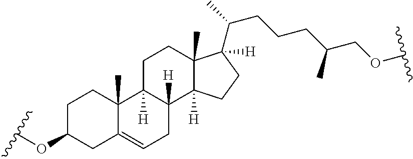

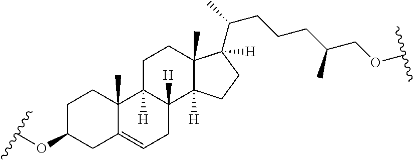

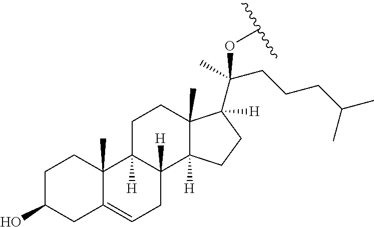

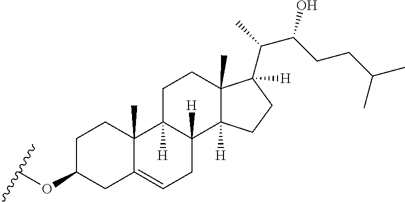

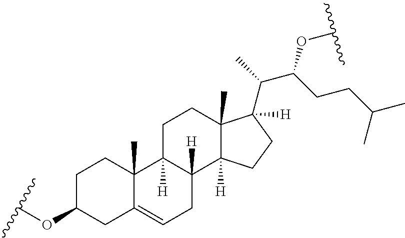

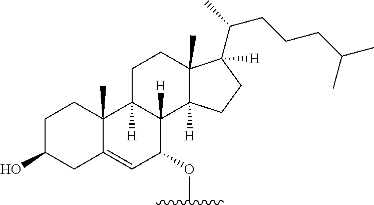

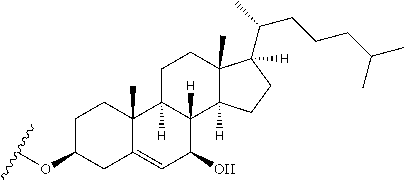

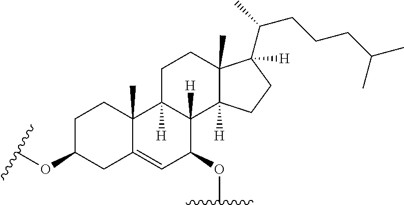

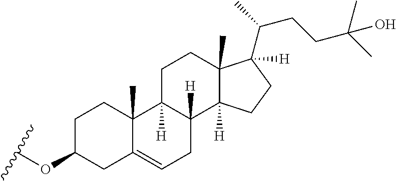

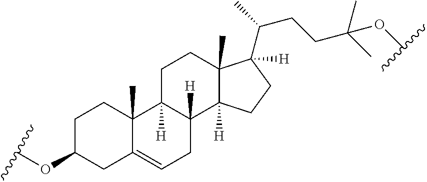

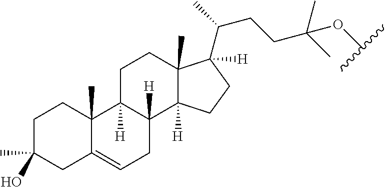

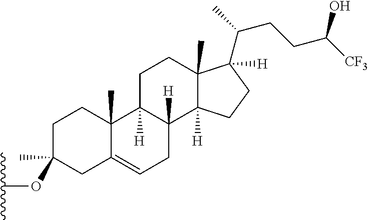

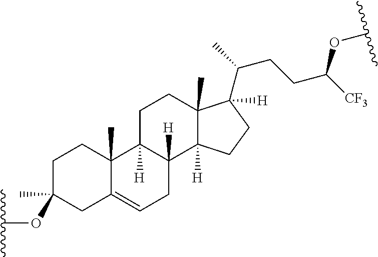

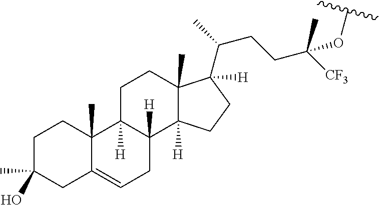

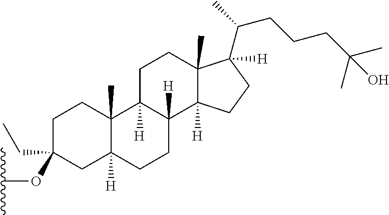

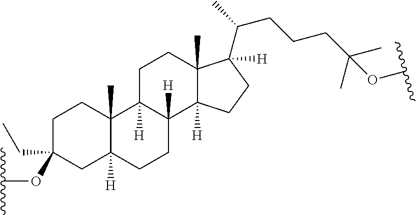

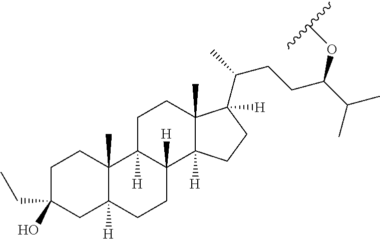

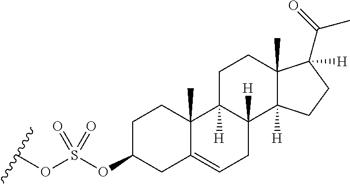

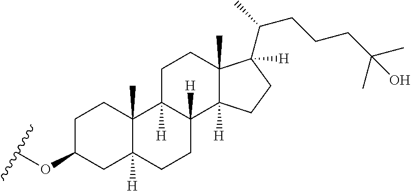

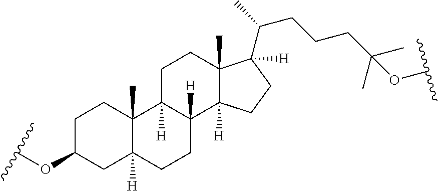

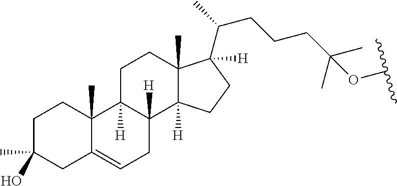

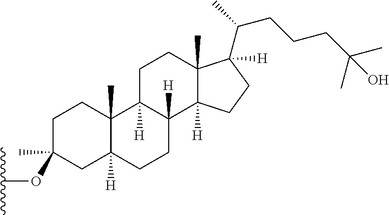

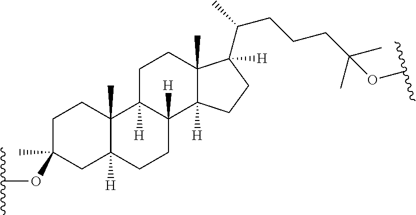

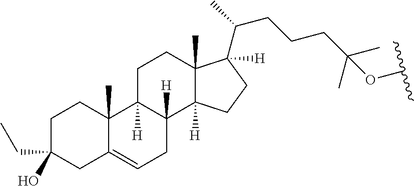

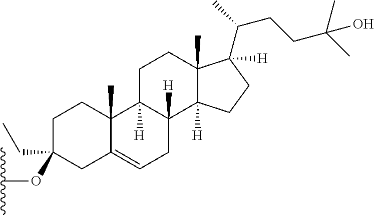

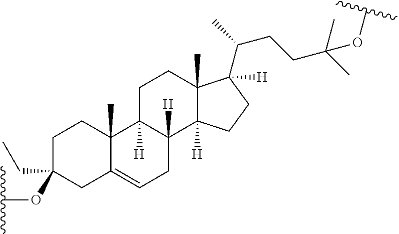

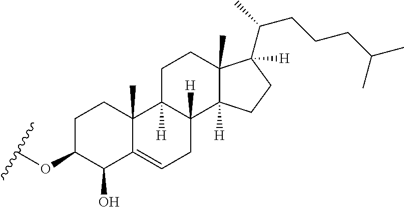

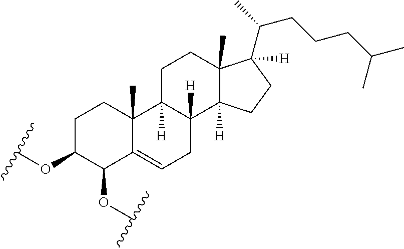

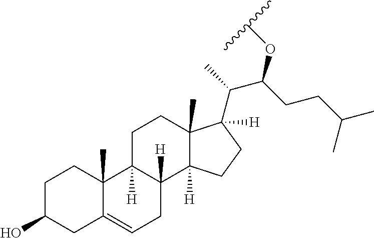

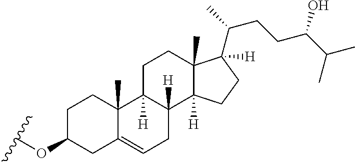

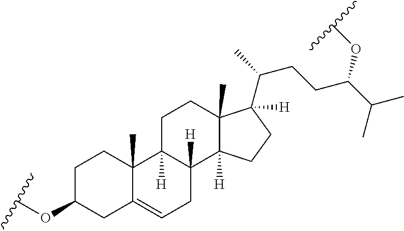

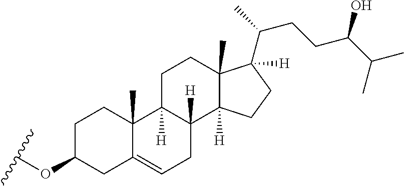

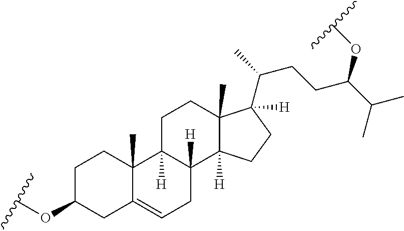

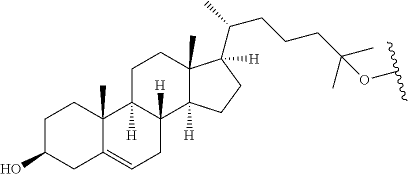

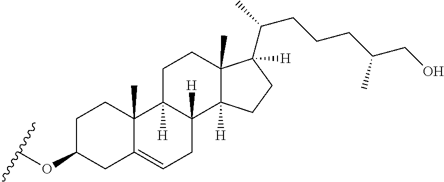

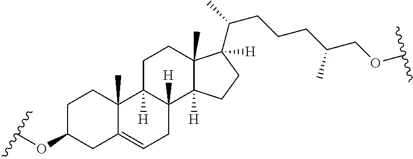

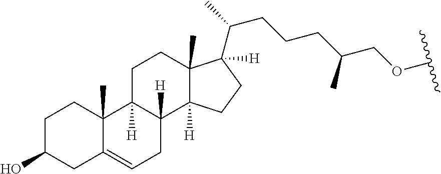

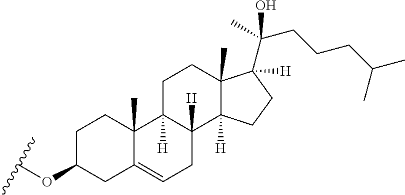

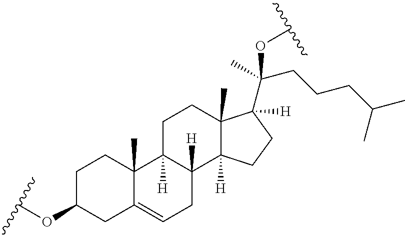

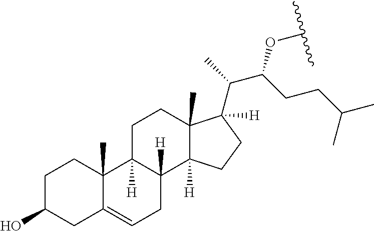

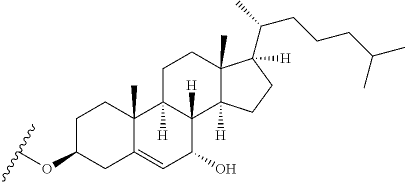

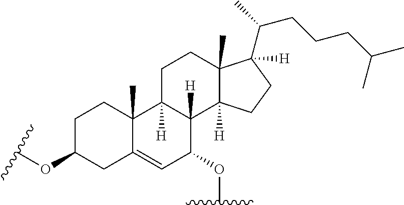

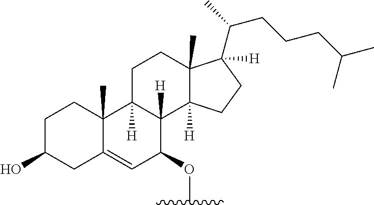

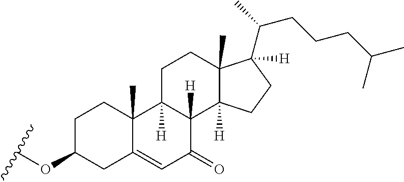

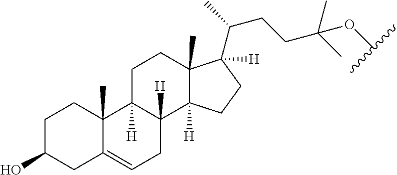

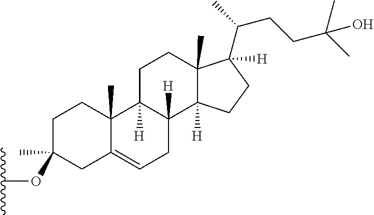

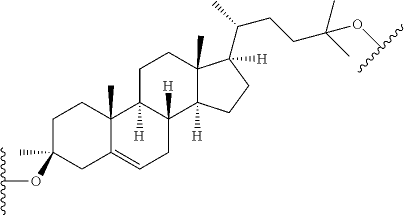

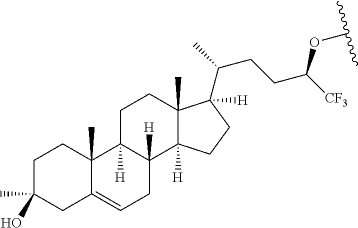

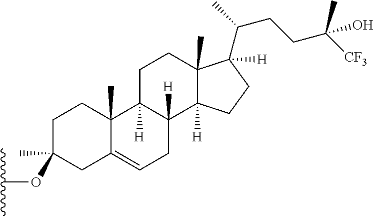

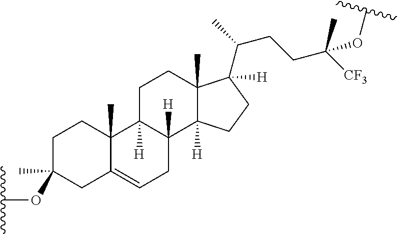

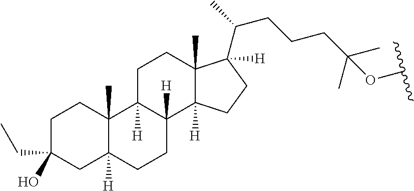

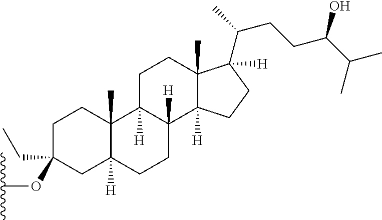

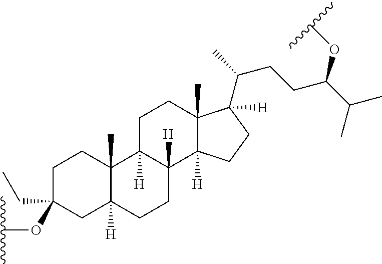

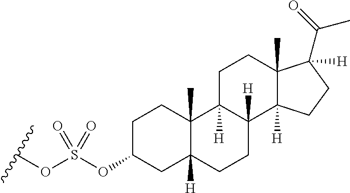

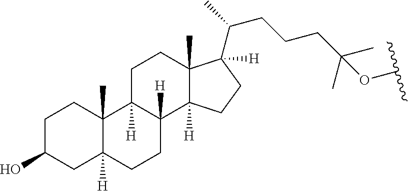

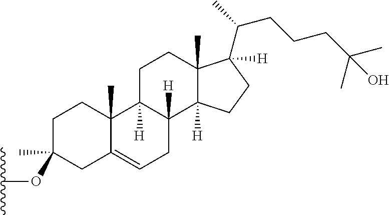

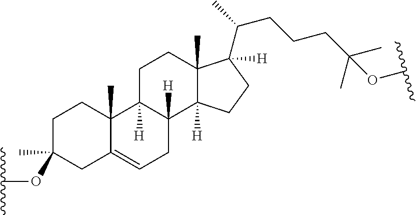

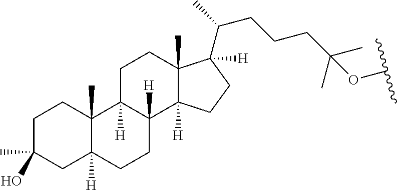

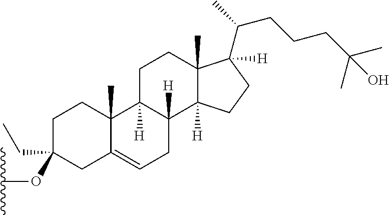

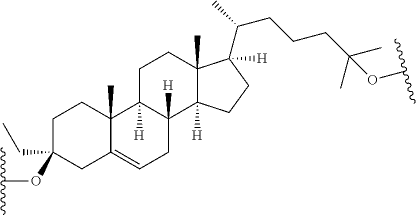

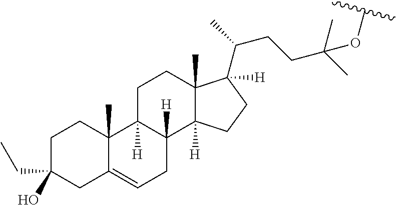

For the sake of clarity, the depiction of brackets and the integer p is 1 or 2 in Formula (I)

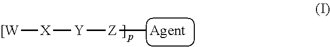

means that either one or two

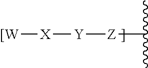

moieties may be covalently attached to the Agent. The attachment of

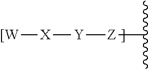

to the Agent may be at any available modifiable nitrogen, oxygen, or sulfur atom in the Agent. Each wavy bond
( [symbol] ) in the structures depicted herein defines the point of attachment.

In some embodiments, the disclosure provides a compound of Formula (II):

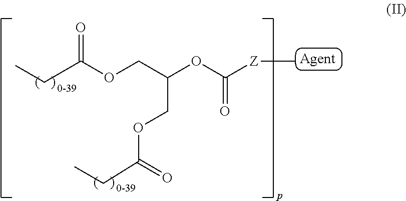

or a pharmaceutically acceptable salt thereof, wherein each of Z, p, and Agent are as defined herein.

In some embodiments, the disclosure provides a compound of Formula (III):

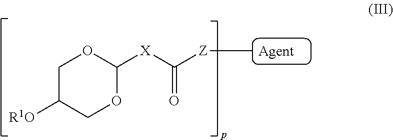

or a pharmaceutically acceptable salt thereof, wherein each of R¹, X, Z, p, and Agent are as defined herein.

In some embodiments, the disclosure provides a compound of Formula (IV):

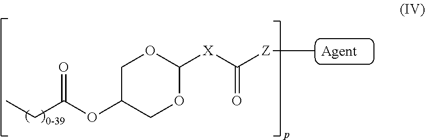

or a pharmaceutically acceptable salt thereof, wherein each of X, Z, p, and Agent are as defined herein.

In some embodiments, the disclosure provides a compound of Formula (V):

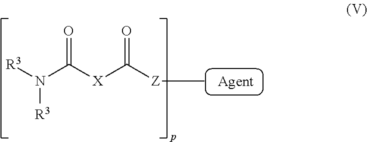

or a pharmaceutically acceptable salt thereof, wherein each of R³, X, Z, p, and Agent are as defined herein.

In some embodiments, the disclosure provides a compound of Formula (VI):

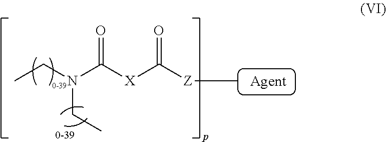

or a pharmaceutically acceptable salt thereof, wherein each of X, Z, p, and Agent are as defined herein.

In some embodiments, the disclosure provides a compound of Formula (VII):

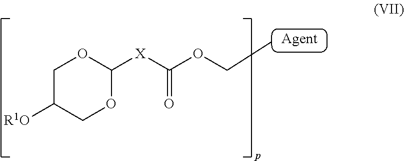

or a pharmaceutically acceptable salt thereof, wherein each of R¹, X, p, and Agent are as defined herein.

In some embodiments, the disclosure provides a compound of Formula (VIII):

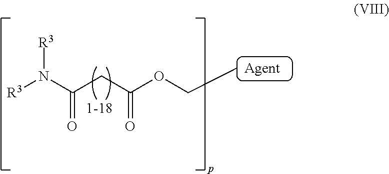

or a pharmaceutically acceptable salt thereof, wherein each of R³, p, and Agent are as defined herein.

In some embodiments, the disclosure provides a compound of Formula (IX):

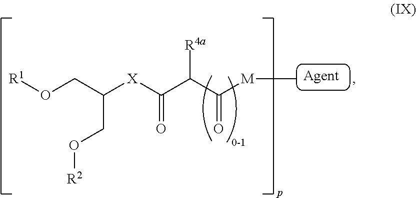

or a pharmaceutically acceptable salt thereof, wherein each of R¹, R², R^{4a}, X, M, p, and Agent are as defined herein.

In some embodiments, the disclosure provides a compound of of Formula (X):

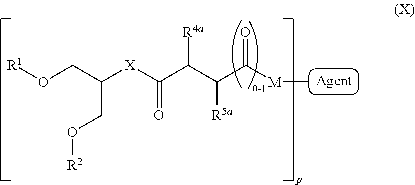

or a pharmaceutically acceptable salt thereof, wherein each of R¹, R², R^{4a}, R^{5a}, M, X, p, and Agent are as defined herein.

In some embodiments, the disclosure provides a compound of Formula (XI):

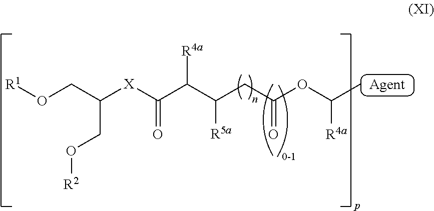

or a pharmaceutically acceptable salt thereof, wherein each of R¹, R², R^{4a}, R^{5a}, X, n, p, and Agent are as defined herein.

In some embodiments, the disclosure provides a compound of Formula (XII):

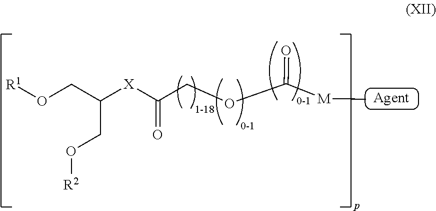

or a pharmaceutically acceptable salt thereof, wherein each of R¹, R², X, M, p and Agent are as defined herein.

In some embodiments, the disclosure provides a compound of Formula (XIII):

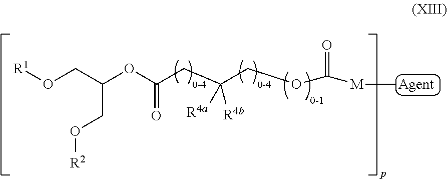

or a pharmaceutically acceptable salt thereof, wherein each of R¹, R², R^{4a}, R^{4b}, M, p and Agent are as defined herein.

In some embodiments, the disclosure provides a compound of Formula (XIV):

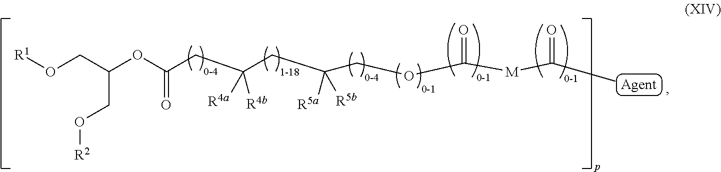

or a pharmaceutically acceptable salt thereof, wherein each of R¹, R², R^{4a}, R^{4b}, R^{5a}, R^{5b}, M, p and Agent are as defined herein.

In some embodiments, the disclosure provides a compound of Formula (XV):

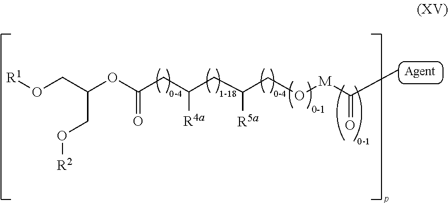

or a pharmaceutically acceptable salt thereof, wherein each of R¹, R², R^{4a}, R^{5a}, M, p and Agent are as defined herein.

In some embodiments, the disclosure provides a compound of Formula (XVI):

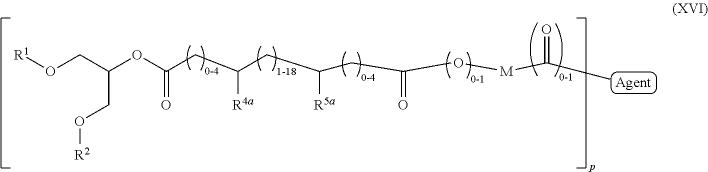

or a pharmaceutically acceptable salt thereof, wherein each of R¹, R², R^{4a}, R^{5a}, M, p and Agent are as defined herein.

In some embodiments, the disclosure provides a compound of Formula (XVII):

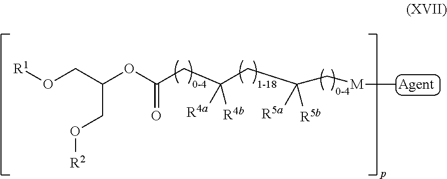

or a pharmaceutically acceptable salt thereof, wherein each of R¹, R², R^{4a}, R^{4b}, R^{5a}, R^{5b}, M, p and Agent are as defined herein.

In some embodiments, the disclosure provides a compound of Formula (XVIII):

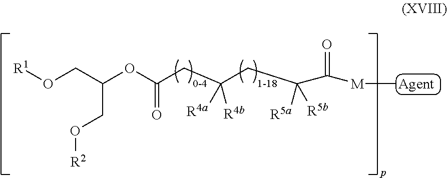

or a pharmaceutically acceptable salt thereof, wherein each of R¹, R², R^{4a}, R^{4b}, R^{5a}, R^{5b}, M, p and Agent are as defined herein.

In some embodiments, the disclosure provides a compound of Formula (XIX):

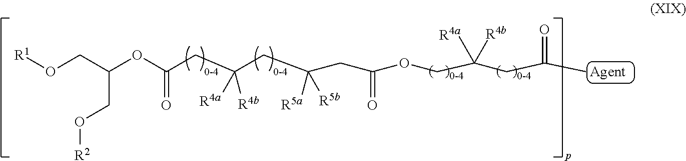

or a pharmaceutically acceptable salt thereof, wherein each of R¹, R², R^{4a}, R^{4b}, R^{5a}, R^{5b}, p and Agent are as defined herein.

In some embodiments, the disclosure provides a compound of Formula (XX):

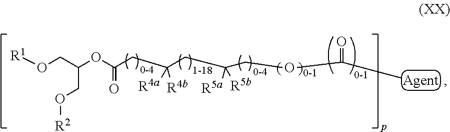

or a pharmaceutically acceptable salt thereof, wherein each of R¹, R², R^{4a}, R^{4b}, R^{5a}, R^{5b}, p, and Agent are as defined herein.

In some embodiments, the disclosure provides a compound of Formula (XXI):

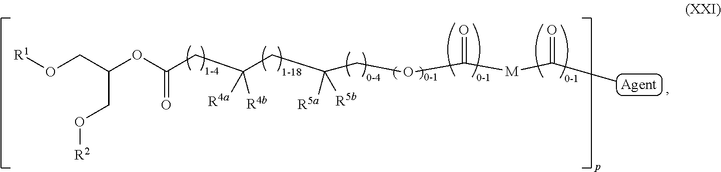

or a pharmaceutically acceptable salt thereof, wherein each of R¹, R², R^{4a}, R^{4b}, R^{5a}, R^{5b}, M, p, and Agent are as defined herein.

In some embodiments, the disclosure provides a compound of Formula (XXII):

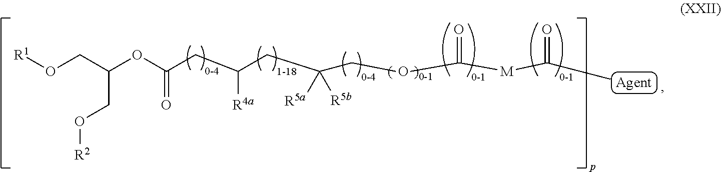

or a pharmaceutically acceptable salt thereof, wherein each of R¹, R², R^{4a}, R^{5a}, R^{5b}, M, p, and Agent are as defined herein.

In some embodiments, the disclosure provides a compound of Formula (XXIII):

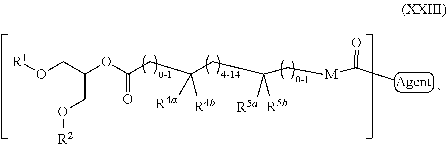

or a pharmaceutically acceptable salt thereof, wherein each of R¹, R², R^{4a}, R^{4b}, R^{5a}, R^{5b}, M, p, and Agent are as defined herein.

In some embodiments, the disclosure provides a compound of Formula (XXIV):

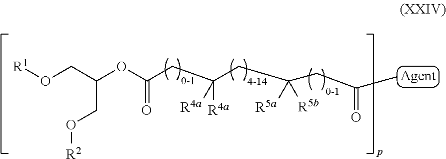

or a pharmaceutically acceptable salt thereof, wherein each of R¹, R², R^{4a}, R^{4b}, R^{5a}, R^{5b}, p, and Agent are as defined herein.

In some embodiments, the disclosure provides a compound of Formula (XXV):

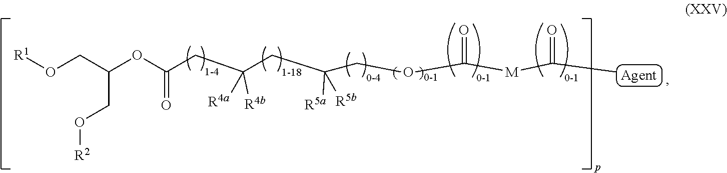

or a pharmaceutically acceptable salt thereof, wherein each of R¹, R², R^{4a}, R^{4b}, R^{5a}, R^{5b}, M, p and Agent are as defined herein.

In some embodiments, the disclosure provides a compound of Formula (XXVI):

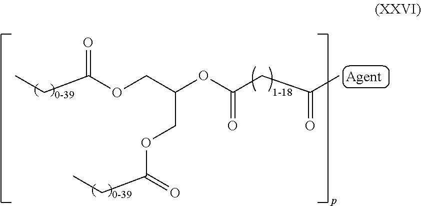

or a pharmaceutically acceptable salt thereof, wherein each of p and Agent are as defined herein.

In some embodiments, the disclosure provides a compound of Formula (XXVII):

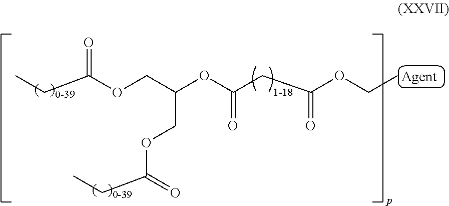

or a pharmaceutically acceptable salt thereof, wherein each of p and Agent are as defined herein.

In any of the formulae disclosed herein, when a range of numbers, such as 0-1, 0-4, 1-18, etc. is disclosed, individual integers within the range are also specifically disclosed. Thus, a range of 0-1 includes 0 and 1. The range of 0-4 includes 0, 1, 2, 3, and 4. The range 1-18 includes 1, 2, 3, 4, 5, 6, 7, 8, 9, 10, 11, 12, 13, 14, 15, 16, 17, and 18. Where more than one range is disclosed in a formula, each range is independently and optionally selected from the disclosed range.

In one aspect, the disclosure provides any one of Compounds 1-2201 shown in any one of Table 2, Table 3, Table 4, Table 5, Table 6, Table 7, Table 8, Table 9, Table 10, Table 10, Table 11, Table 12, Table 13, Table 14, Table 15, Table 16, Table 17, Table 18, Table 19, Table 20, Table 21, Table 22, Table 23, Table 24, Table 25, Table 26, Table 27, Table 28, Table 29, Table 30, Table 31, or Table 32, pharmaceutically acceptable salts thereof, deuterated variants thereof or combinations thereof. In some embodiments, the compound is selected from any one of Compounds 1-2201, or a pharmaceutically acceptable salt thereof. In some embodiments, the compound is selected from any one of Compounds 1-2201. In some embodiments, the compound is selected from a pharmaceutically acceptable salt of any one of Compounds 1-2201. In some embodiments, the compound is selected from a deuterated variant of any one of Compounds 1-2201. In some embodiments, the compound is selected from a deuterated variant of a pharmaceutically acceptable salt of any one of Compounds 1-2201.

In some embodiments, the compound is selected from any of the compounds depicted in in Table 2, pharmaceutically acceptable salts thereof, deuterated variants thereof or combinations thereof.

TABLE 2
Formula (XXVIII)

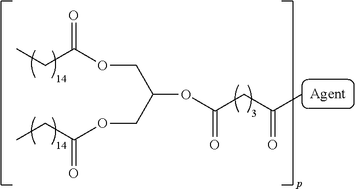

| Cpd. No. | p | 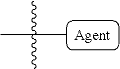 |
|---|---|---|
| 1 | 1 | 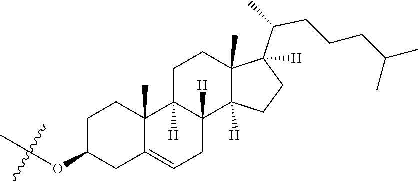 |
| 2 | 1 | 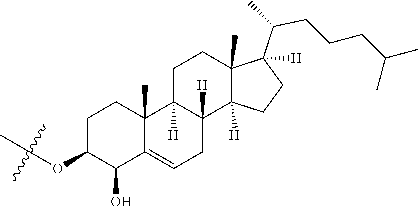 |
| 3 | 1 | 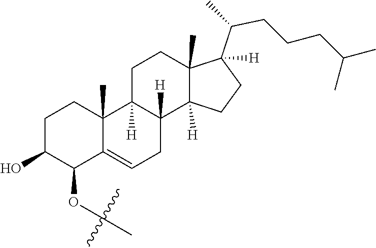 |
| 4 | 2 | 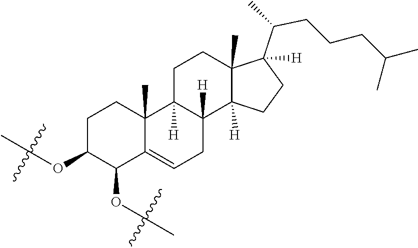 |
| 5 | 1 | 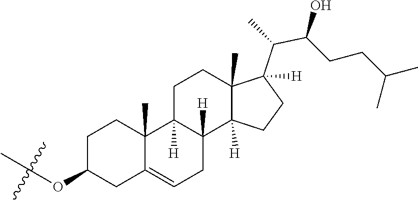 |
| 6 | 1 | 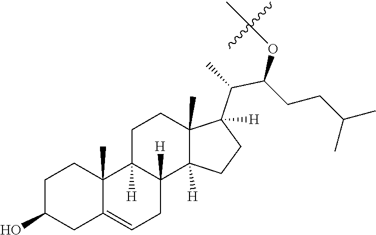 |
| 7 | 2 | 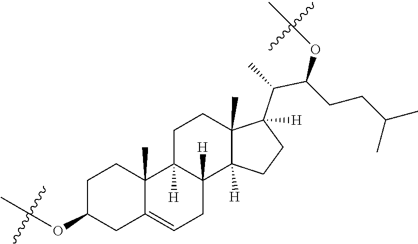 |
| 8 | 1 | 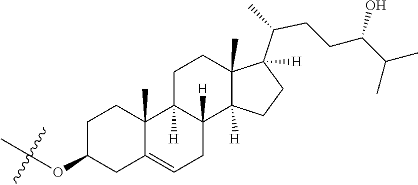 |
| 9 | 1 | 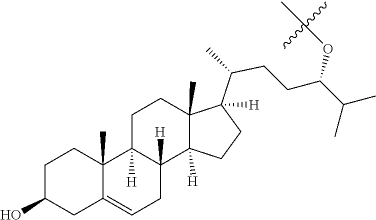 |
| 10 | 2 | 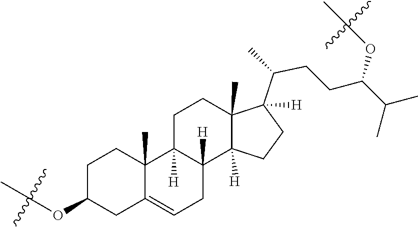 |
| 11 | 1 | 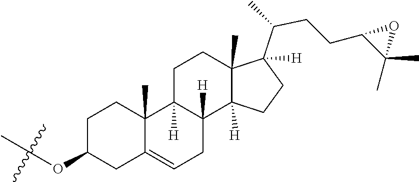 |
| 12 | 1 | 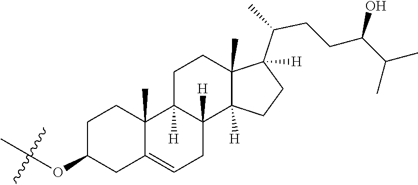 |
| 13 | 1 | 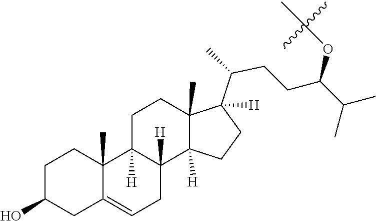 |
| 14 | 2 | 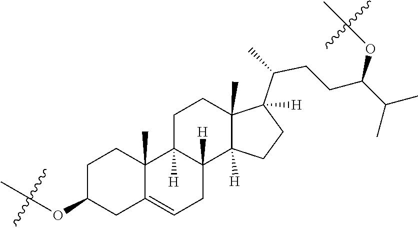 |
| 15 | 1 | 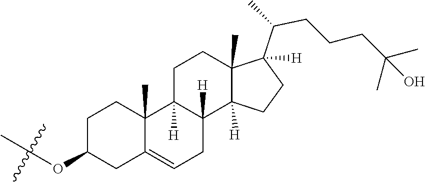 |
| 16 | 1 | 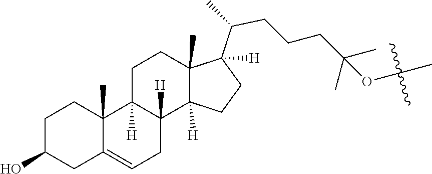 |
| 17 | 2 | 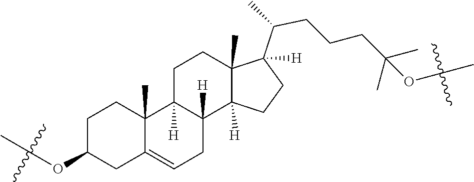 |
| 18 | 1 | 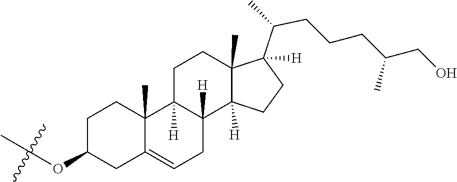 |
| 19 | 1 | 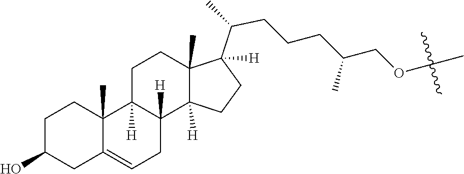 |
| 20 | 2 | 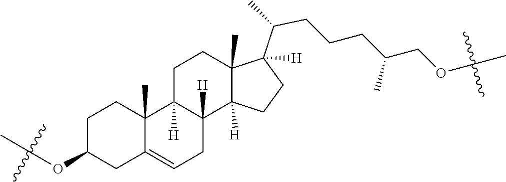 |
| 21 | 1 | 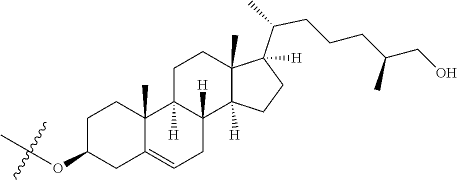 |
| 22 | 1 | 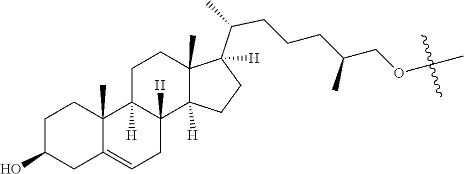 |
| 23 | 2 | 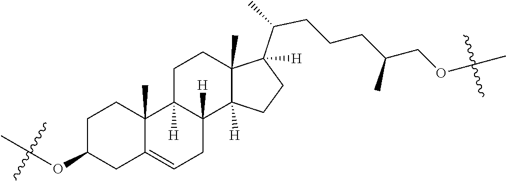 |
| 24 | 1 | 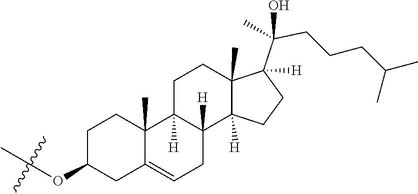 |
| 25 | 1 | 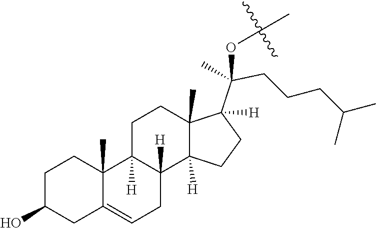 |
| 26 | 2 | 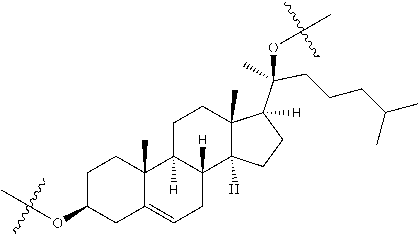 |
| 27 | 1 | 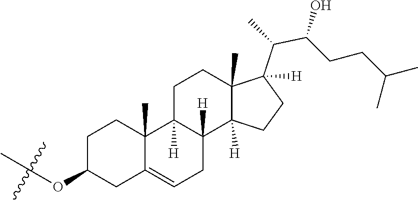 |
| 28 | 1 | 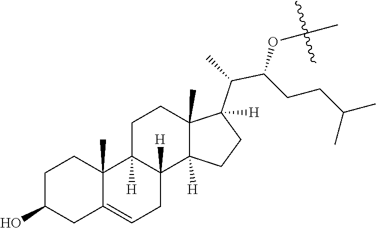 |
| 29 | 2 | 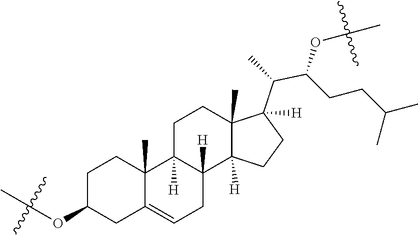 |
| 30 | 1 | 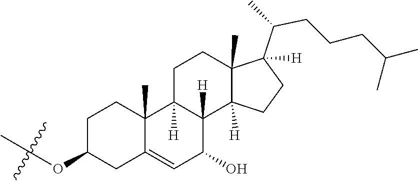 |
| 31 | 1 | 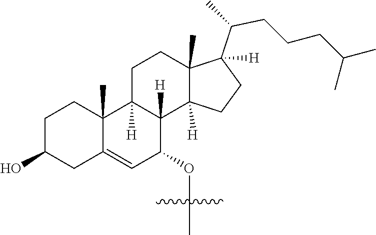 |
| 32 | 2 | 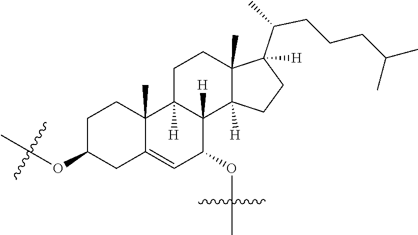 |
| 33 | 1 | 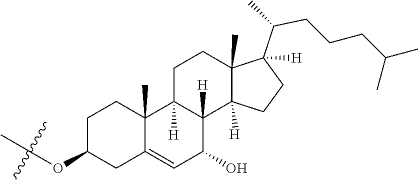 |
| 34 | 1 | 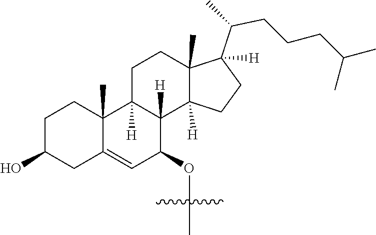 |
| 35 | 2 | 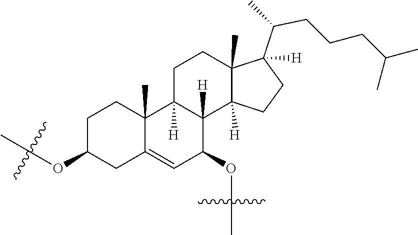 |
| 36 | 1 | 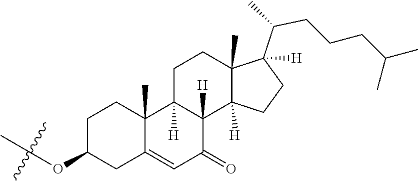 |
| 37 | 1 | 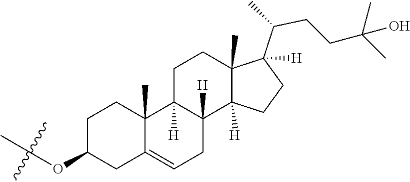 |
| 38 | 1 | 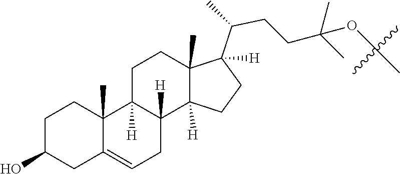 |
| 39 | 2 | 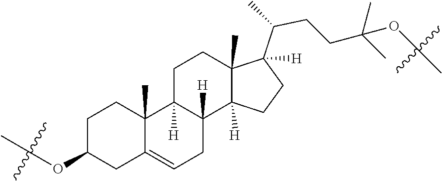 |
| 40 | 1 | 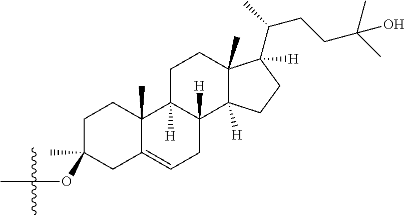 |
| 41 | 1 | 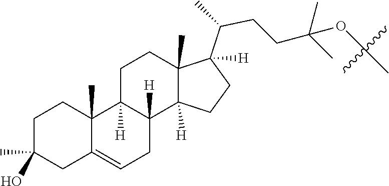 |
| 42 | 2 | 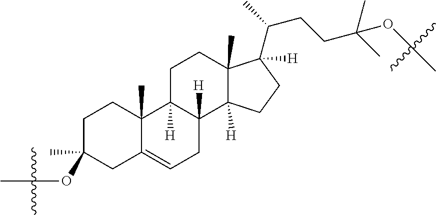 |
| 43 | 1 | 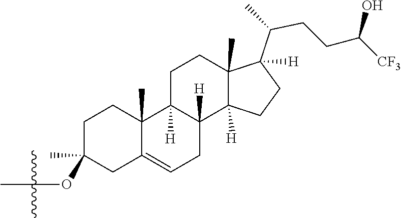 |
| 44 | 1 | 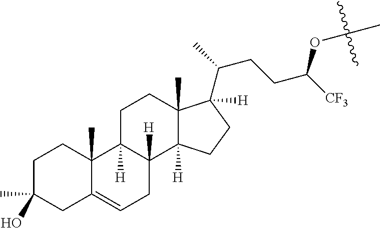 |
| 45 | 2 | 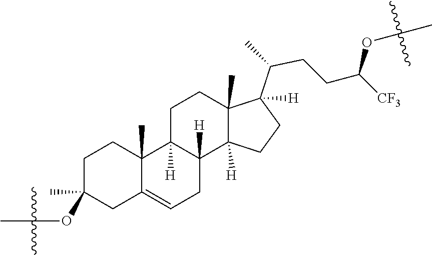 |
| 46 | 1 | 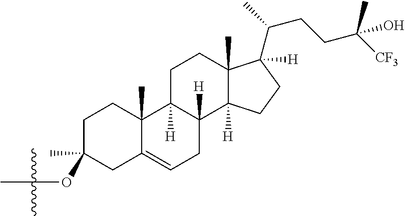 |
| 47 | 1 | 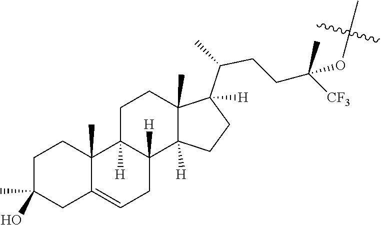 |
| 48 | 2 | 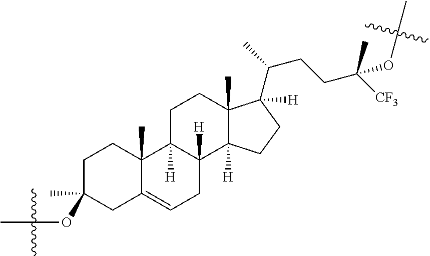 |
| 49 | 1 | 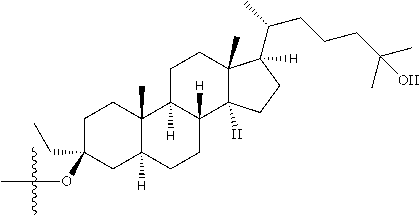 |
| 50 | 1 | 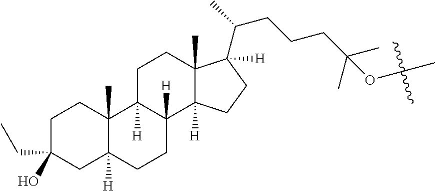 |
| 51 | 2 | 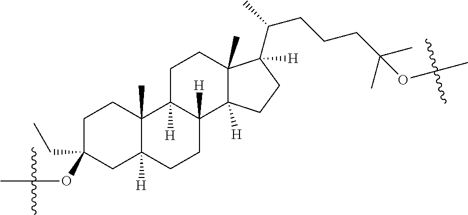 |
| 52 | 1 | 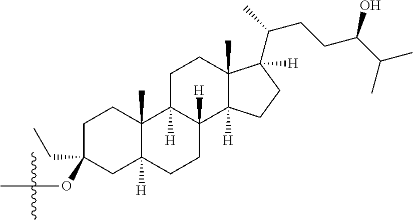 |
| 53 | 1 | 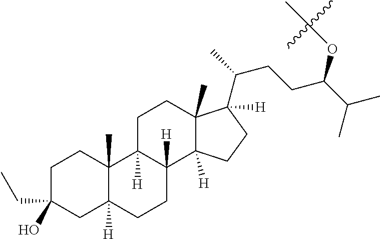 |
| 54 | 2 | 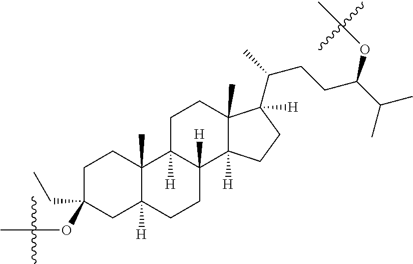 |
| 55 | 1 | 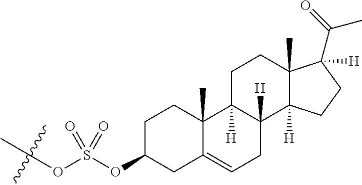 |
| 56 | 1 | 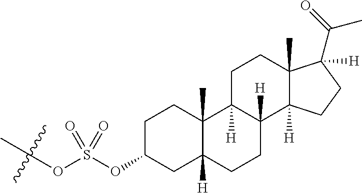 |
| 57 | 1 | 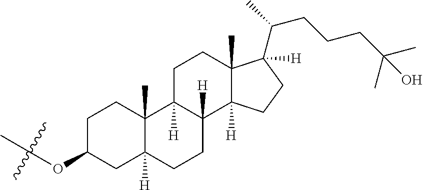 |
| 58 | 1 | 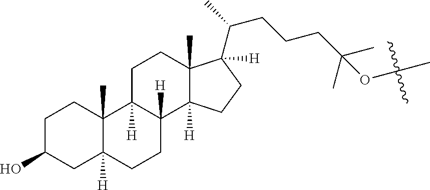 |
| 59 | 2 | 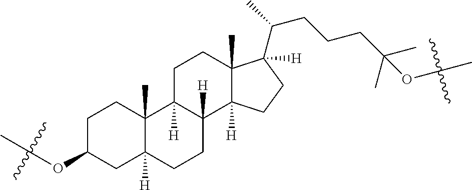 |
| 60 | 1 | 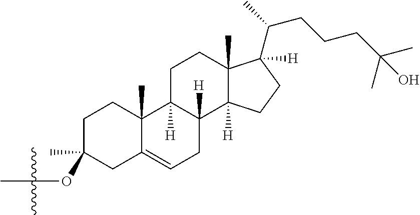 |
| 61 | 1 | 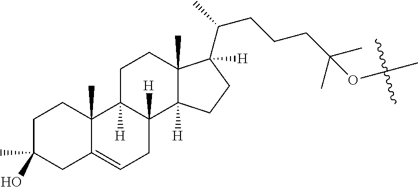 |
| 62 | 2 | 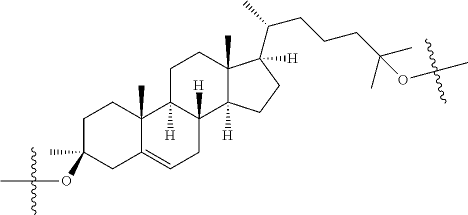 |
| 63 | 1 | 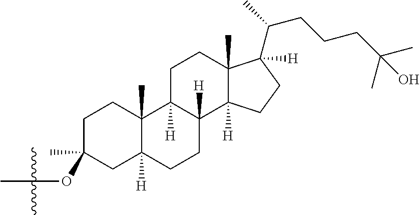 |
| 64 | 1 | 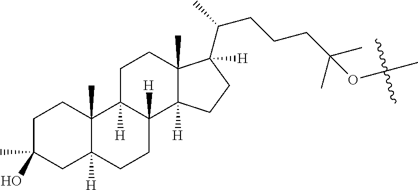 |
| 65 | 2 | 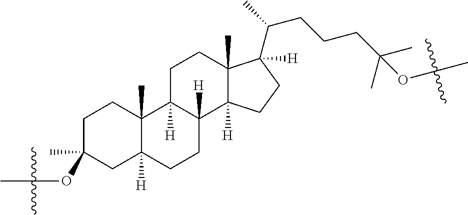 |
| 66 | 1 | 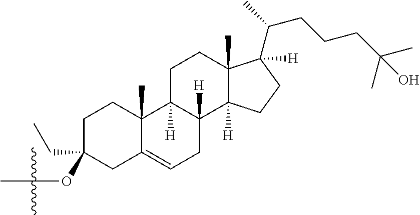 |
| 67 | 1 | 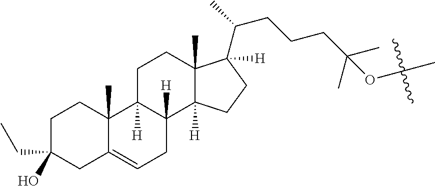 |
| 68 | 2 | 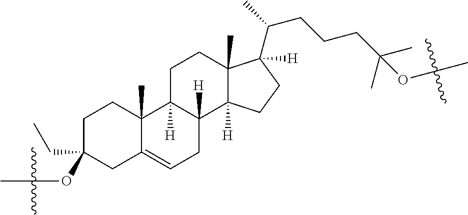 |
| 69 | 1 | 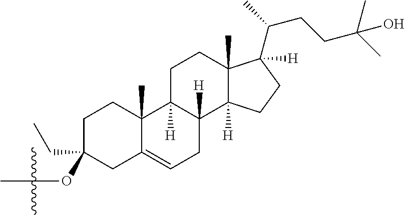 |
| 70 | 1 | 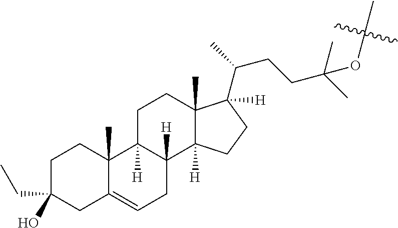 |
| 71 | 2 | 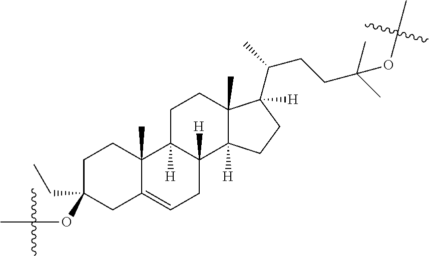 |

In some embodiments, the compound is selected from any of the compounds depicted in in Table 3, pharmaceutically acceptable salts thereof, deuterated variants thereof or combinations thereof.

TABLE 3
Formula (XXVIX)

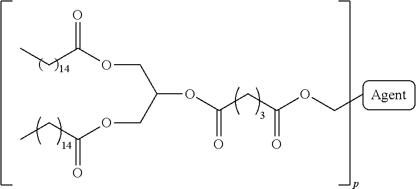

| Cpd. No. | p | 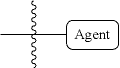 |
|---|---|---|
| 72 | 1 | 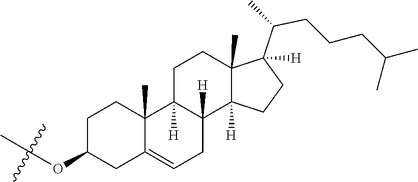 |
| 73 | 1 | 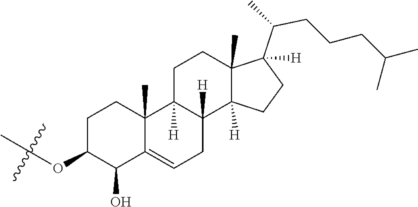 |
| 74 | 1 | 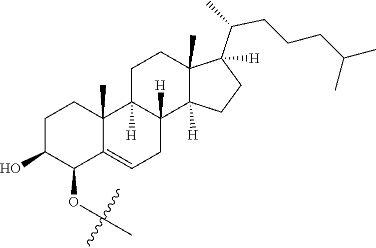 |
| 75 | 2 | 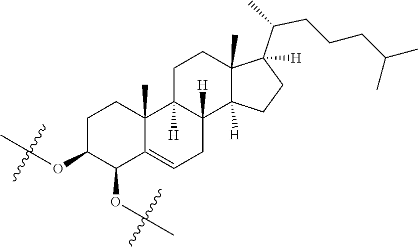 |
| 76 | 1 | 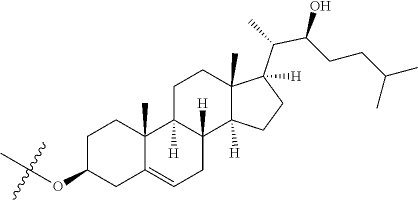 |
| 77 | 1 | 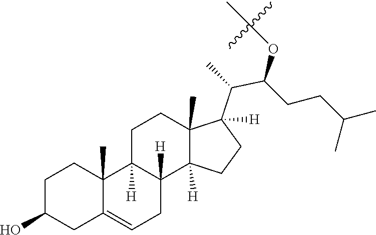 |
| 78 | 2 | 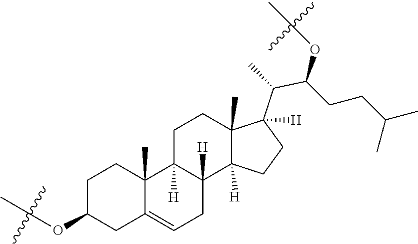 |
| 79 | 1 | 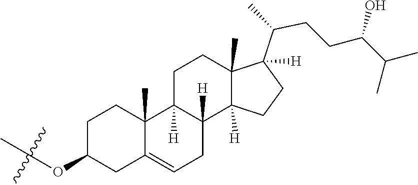 |
| 80 | 1 | 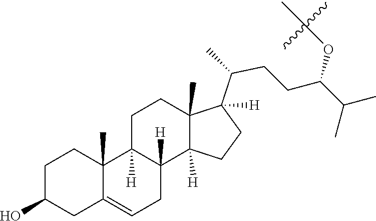 |
| 81 | 2 | 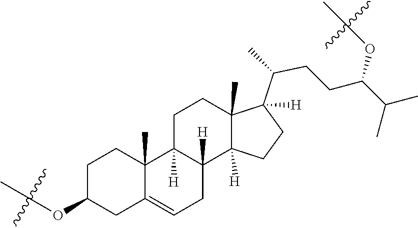 |
| 82 | 1 | 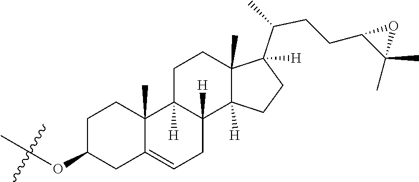 |
| 83 | 1 | 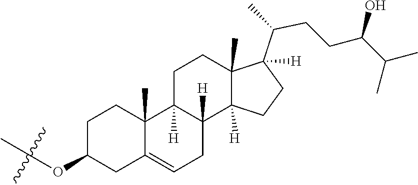 |
| 84 | 1 | 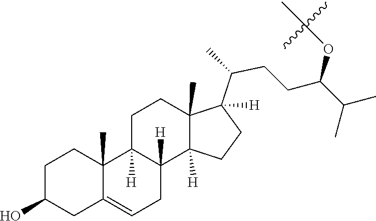 |
| 85 | 2 | 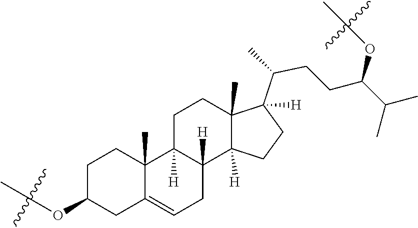 |
| 86 | 1 | 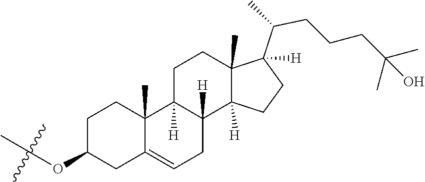 |
| 87 | 1 | 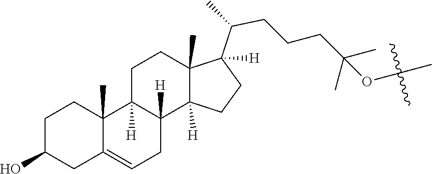 |
| 88 | 2 | 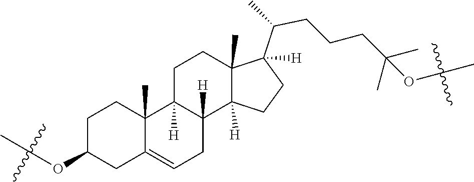 |
| 89 | 1 | 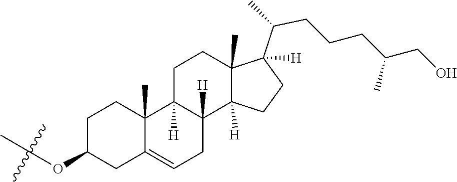 |
| 90 | 1 | 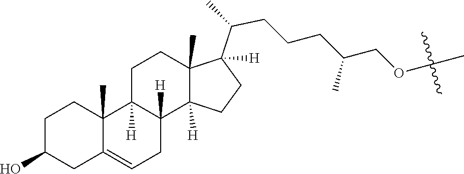 |
| 91 | 2 | 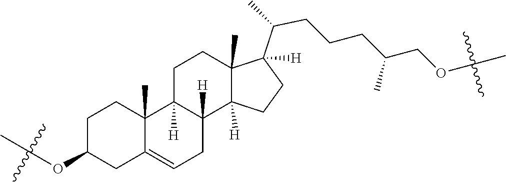 |
| 92 | 1 | 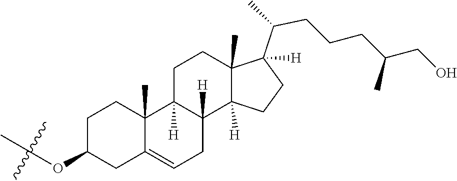 |
| 93 | 1 | 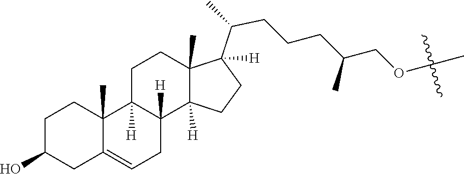 |
| 94 | 2 | 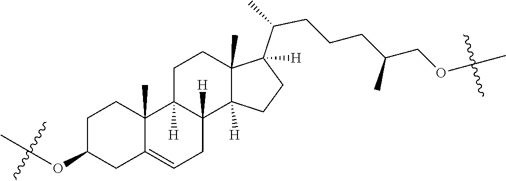 |
| 95 | 1 | 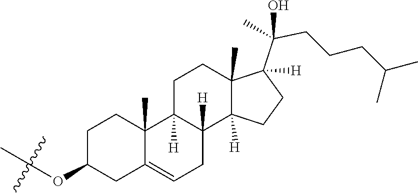 |
| 96 | 1 | 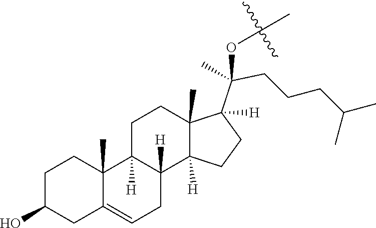 |
| 97 | 2 | 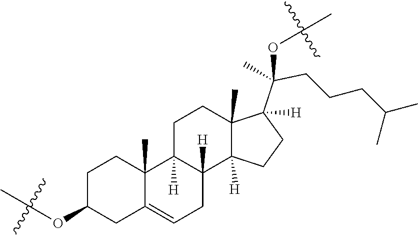 |
| 98 | 1 | 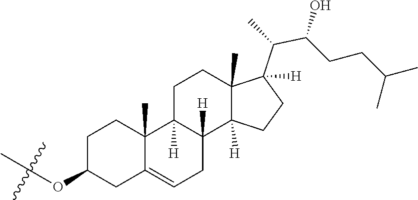 |
| 99 | 1 | 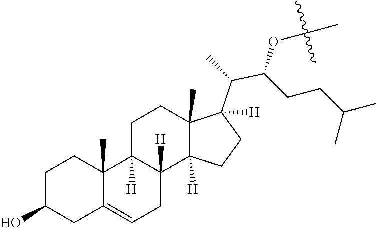 |
| 100 | 2 | 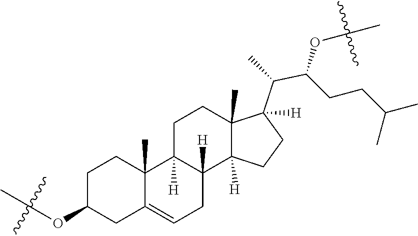 |
| 101 | 1 | 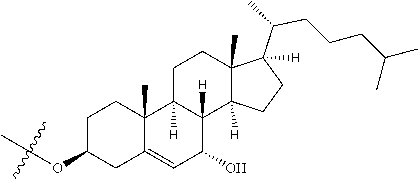 |
| 102 | 1 | 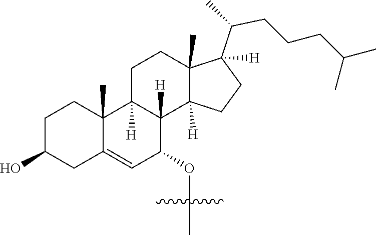 |
| 103 | 2 | 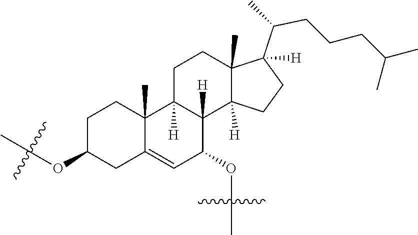 |
| 104 | 1 | 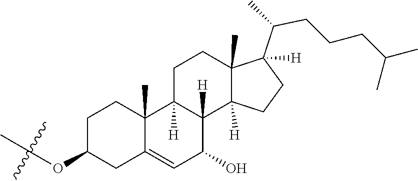 |
| 105 | 1 | 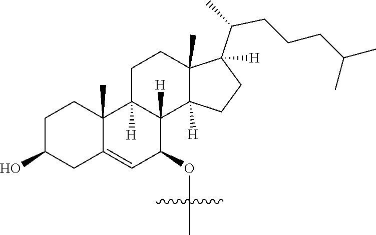 |
| 106 | 2 | 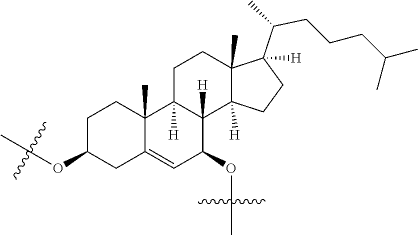 |
| 107 | 1 | 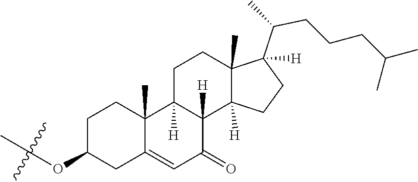 |
| 108 | 1 | 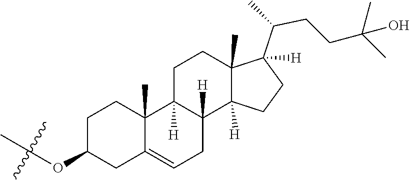 |
| 109 | 1 | 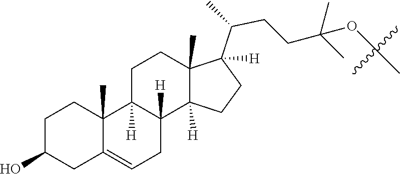 |
| 110 | 2 | 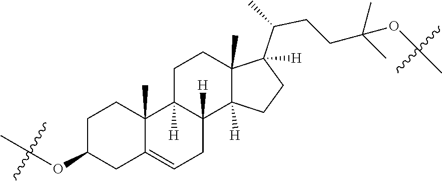 |
| 111 | 1 | 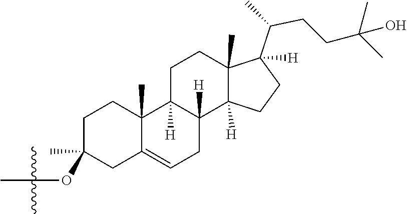 |
| 112 | 1 | 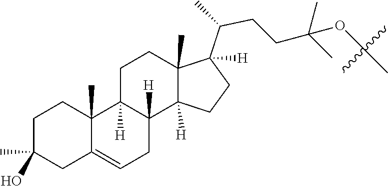 |
| 113 | 2 | 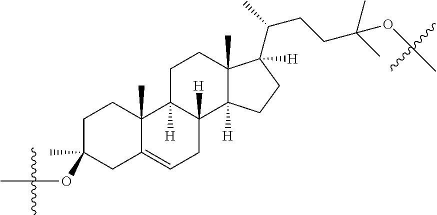 |
| 114 | 1 | 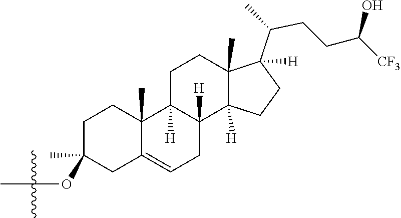 |
| 115 | 1 | 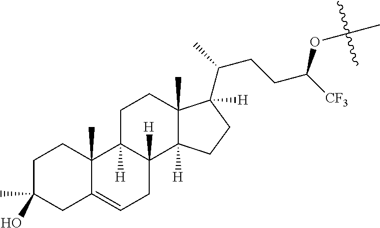 |
| 116 | 2 | 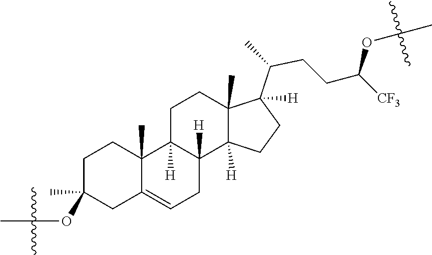 |
| 117 | 1 | 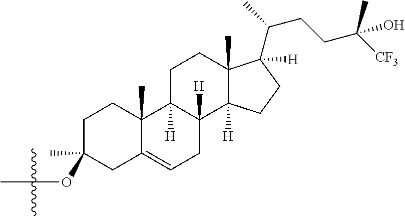 |
| 118 | 1 | 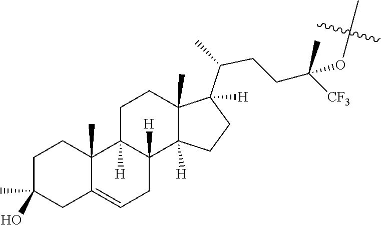 |
| 119 | 2 | 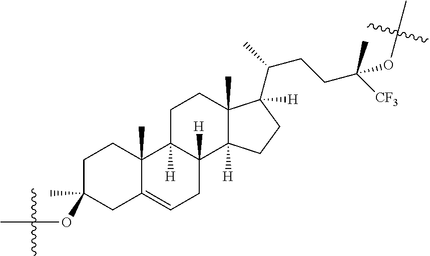 |
| 120 | 1 | 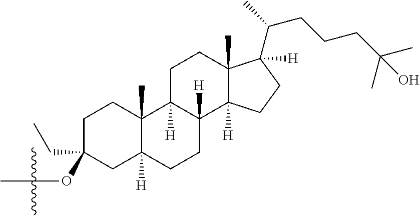 |
| 121 | 1 | 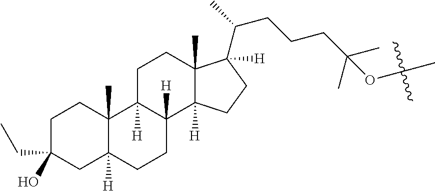 |
| 122 | 2 | 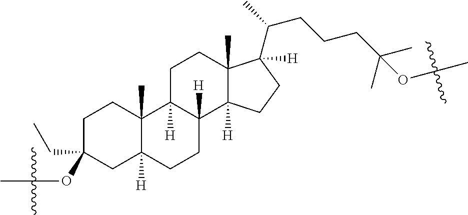 |
| 123 | 1 | 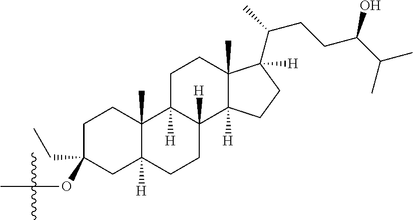 |
| 124 | 1 | 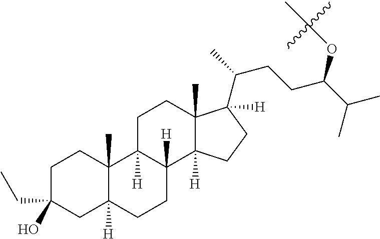 |
| 125 | 2 | 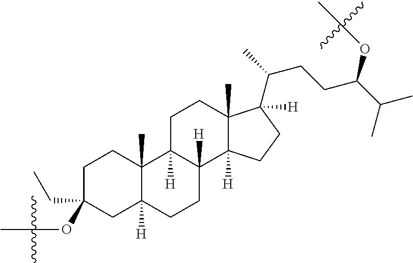 |
| 126 | 1 | 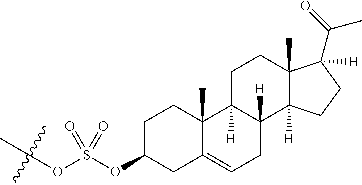 |
| 127 | 1 | 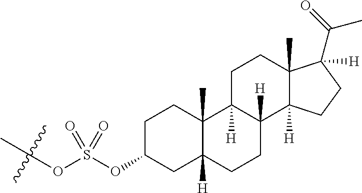 |
| 128 | 1 | 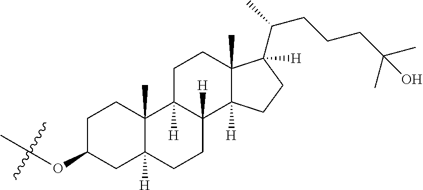 |
| 129 | 1 | 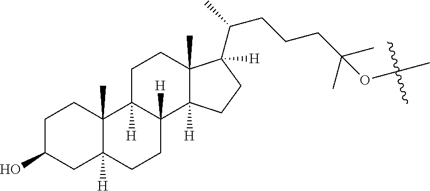 |
| 130 | 2 | 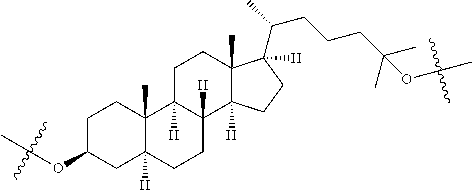 |
| 131 | 1 | 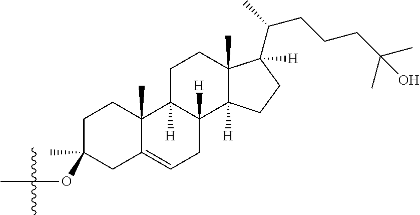 |
| 132 | 1 | 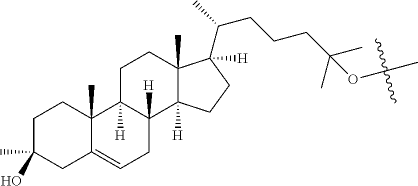 |
| 133 | 2 | 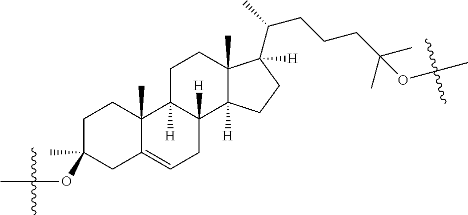 |
| 134 | 1 | 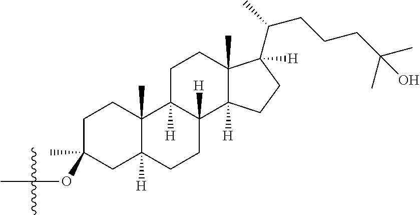 |
| 135 | 1 | 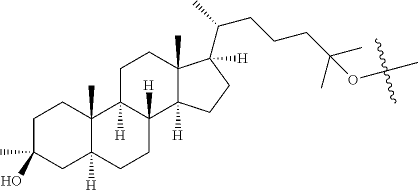 |
| 136 | 2 | 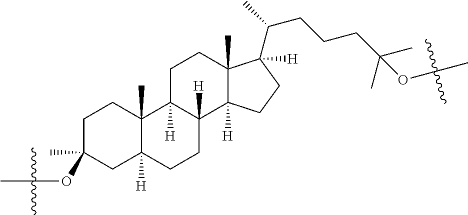 |
| 137 | 1 | 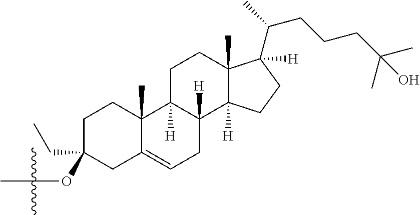 |
| 138 | 1 | 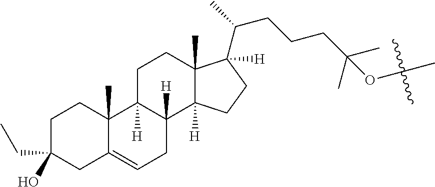 |
| 139 | 2 | 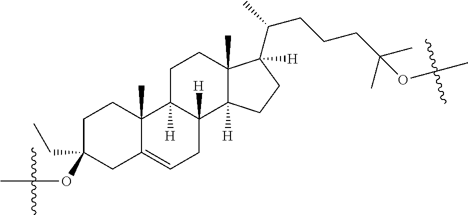 |
| 140 | 1 | 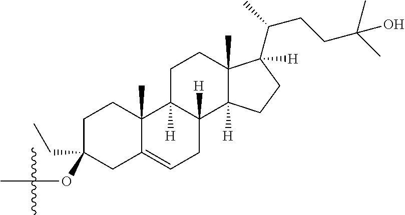 |
| 141 | 1 | 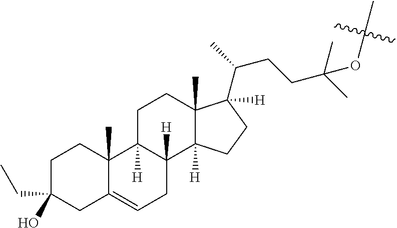 |
| 142 | 2 | 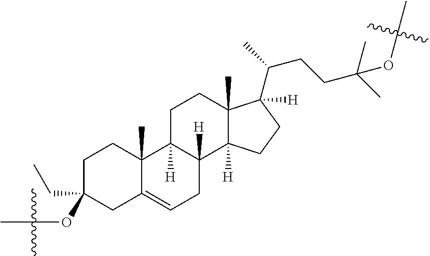 |

In some embodiments, the compound is selected from any of the compounds depicted in in Table 4, pharmaceutically acceptable salts thereof, deuterated variants thereof or combinations thereof.

TABLE 4
Formula (XXX)

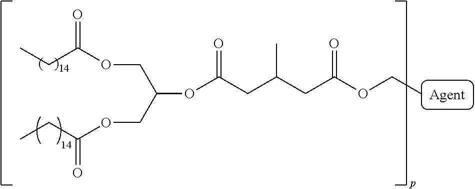

| Cpd. No. | p | 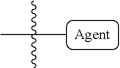 |
|---|---|---|
| 143 | 1 | 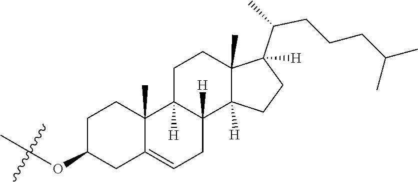 |
| 144 | 1 | 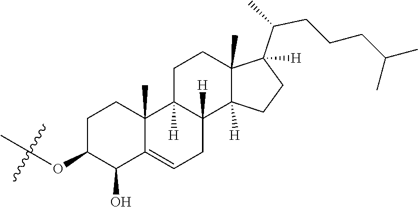 |
| 145 | 1 | 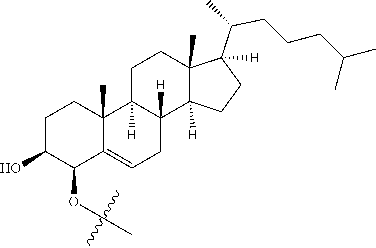 |
| 146 | 2 | 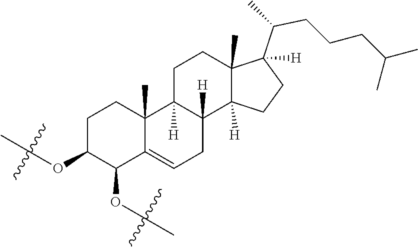 |
| 147 | 1 | 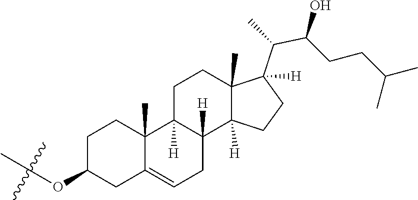 |
| 148 | 1 | 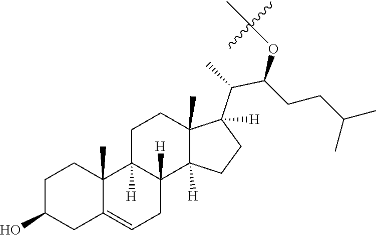 |
| 149 | 2 | 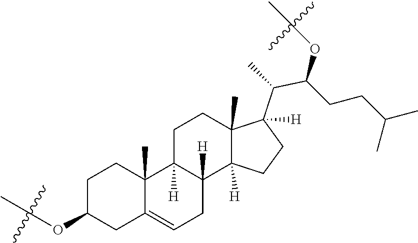 |
| 150 | 1 | 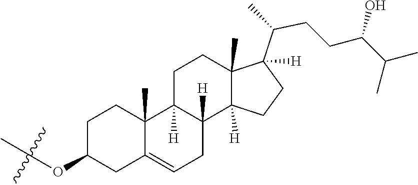 |
| 151 | 1 | 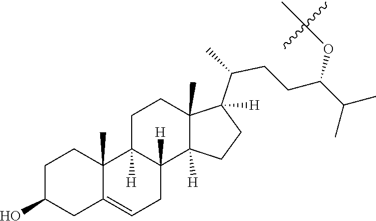 |
| 152 | 2 | 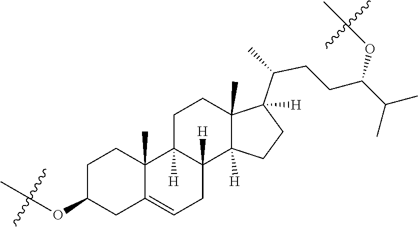 |
| 153 | 1 | 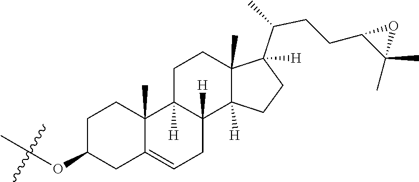 |
| 154 | 1 | 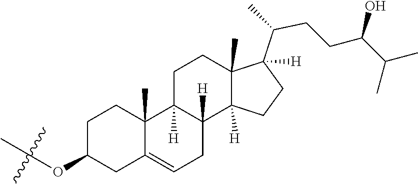 |
| 155 | 1 | 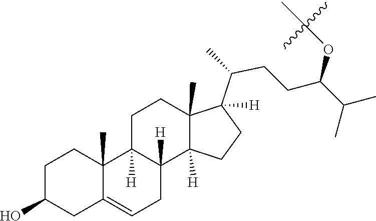 |
| 156 | 2 | 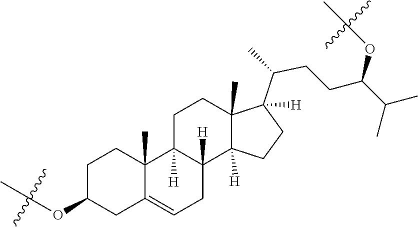 |
| 157 | 1 | 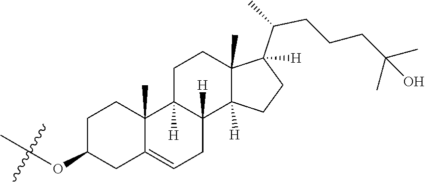 |
| 158 | 1 | 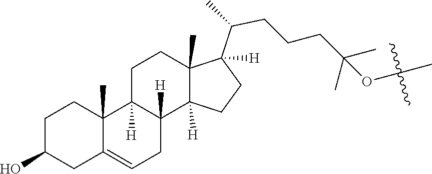 |
| 159 | 2 | 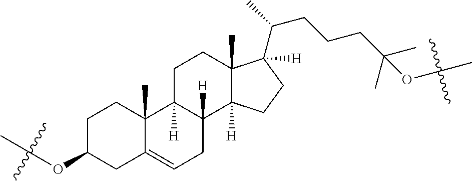 |
| 160 | 1 | 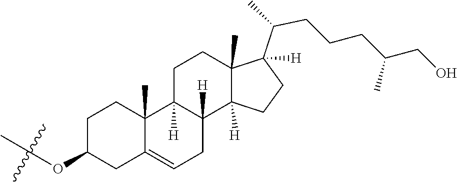 |
| 161 | 1 | 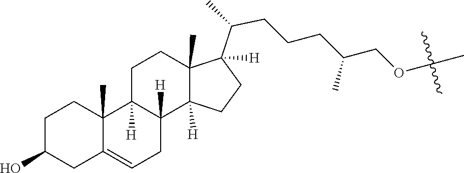 |
| 162 | 2 | 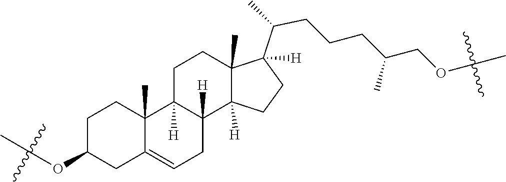 |
| 163 | 1 | 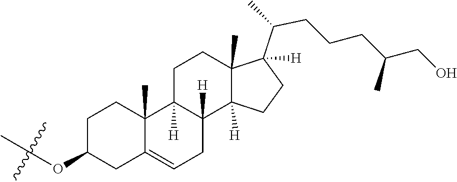 |
| 164 | 1 | 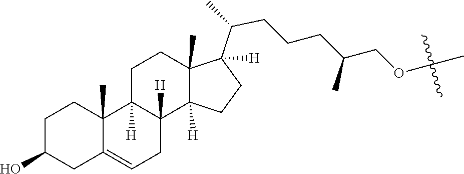 |
| 165 | 2 | 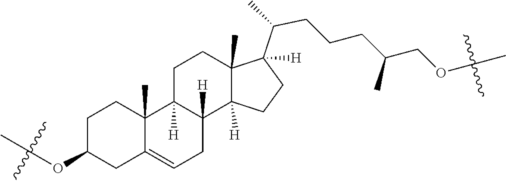 |
| 166 | 1 | 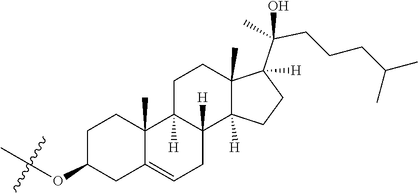 |
| 167 | 1 | 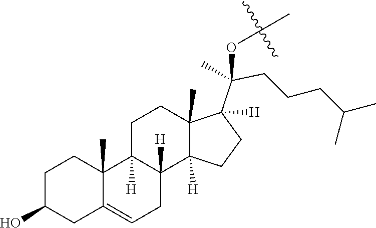 |
| 168 | 2 | 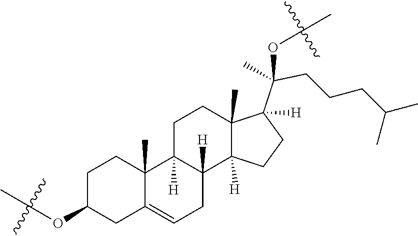 |
| 169 | 1 | 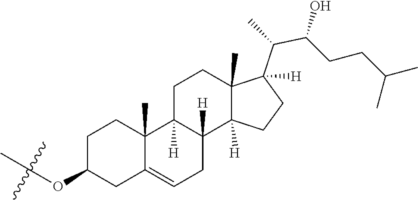 |
| 170 | 1 | 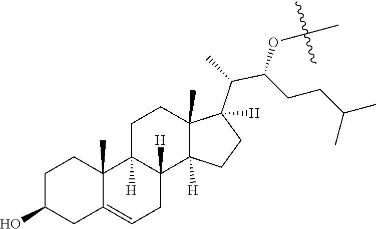 |
| 171 | 2 | 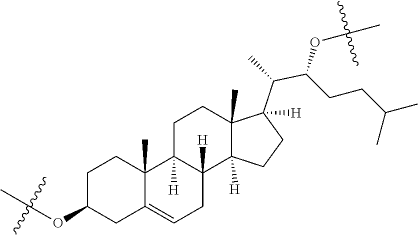 |
| 172 | 1 | 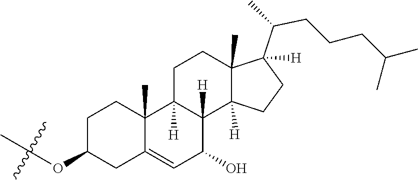 |
| 173 | 1 | 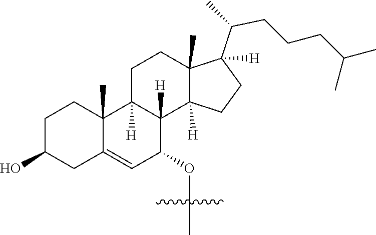 |
| 174 | 2 | 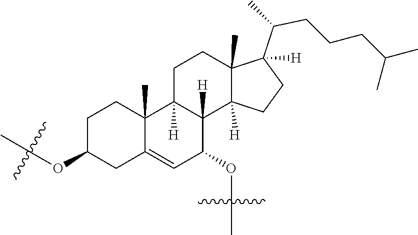 |
| 175 | 1 | 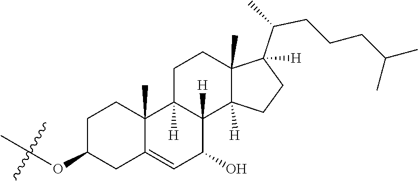 |
| 176 | 1 | 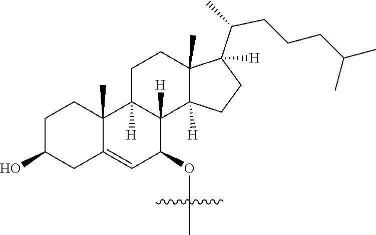 |
| 177 | 2 | 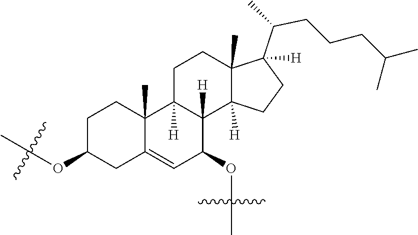 |
| 178 | 1 | 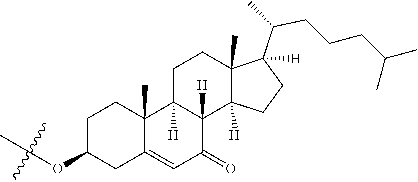 |
| 179 | 1 | 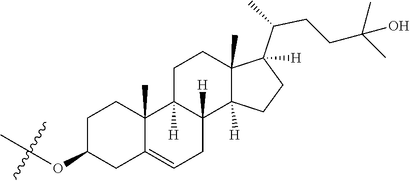 |
| 180 | 1 | 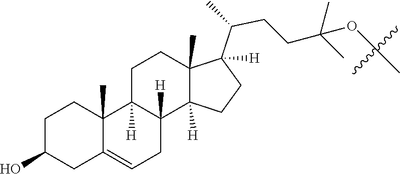 |
| 181 | 2 | 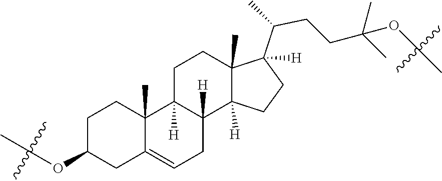 |
| 182 | 1 | 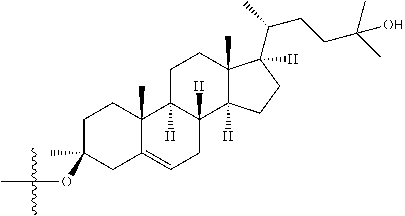 |
| 183 | 1 | 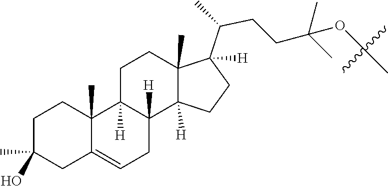 |
| 184 | 2 | 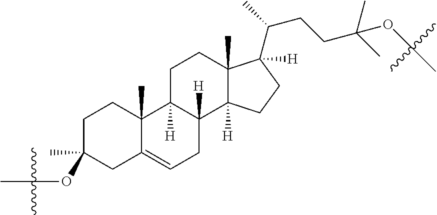 |
| 185 | 1 | 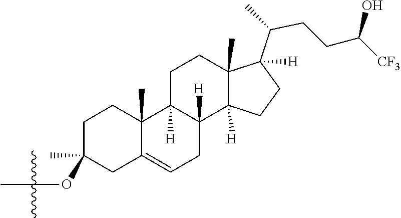 |
| 186 | 1 | 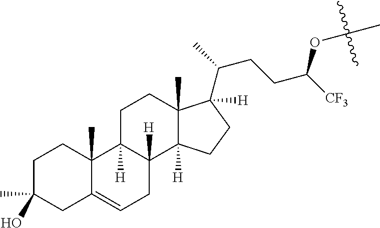 |
| 187 | 2 | 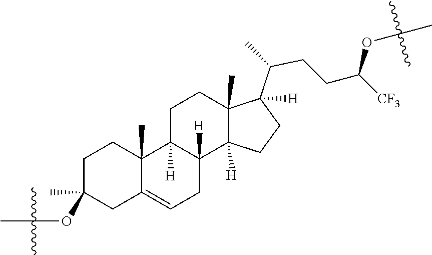 |
| 188 | 1 | 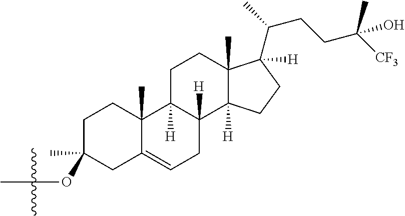 |
| 189 | 1 | 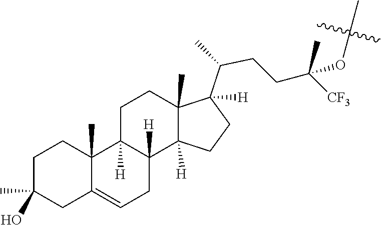 |
| 190 | 2 | 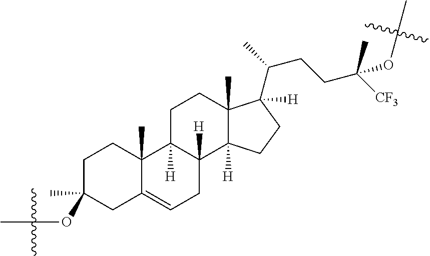 |
| 191 | 1 | 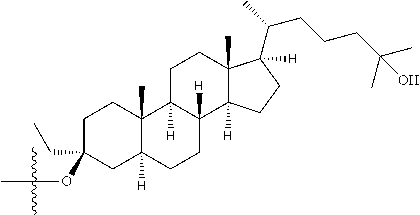 |
| 192 | 1 | 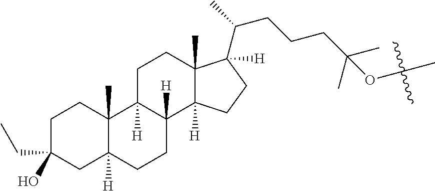 |
| 193 | 2 | 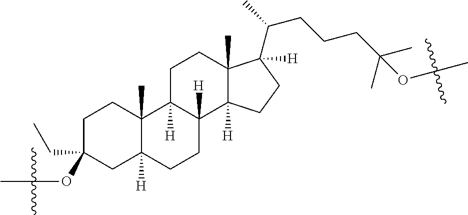 |
| 194 | 1 | 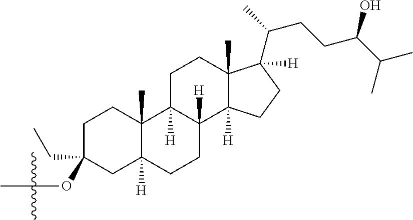 |
| 195 | 1 | 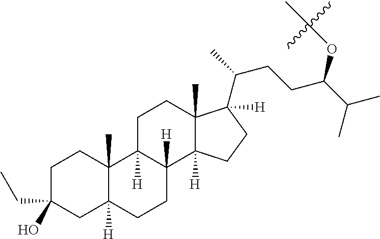 |
| 196 | 2 | 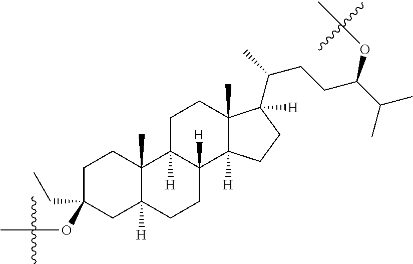 |
| 197 | 1 | 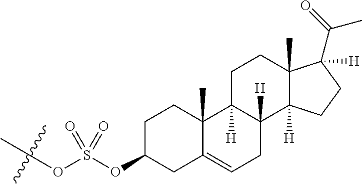 |
| 198 | 1 | 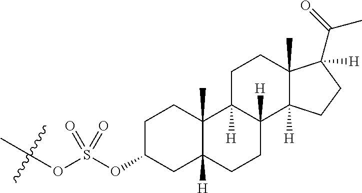 |
| 199 | 1 | 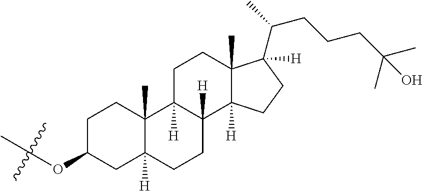 |
| 200 | 1 | 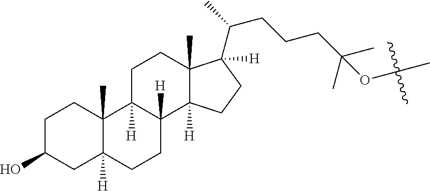 |
| 201 | 2 | 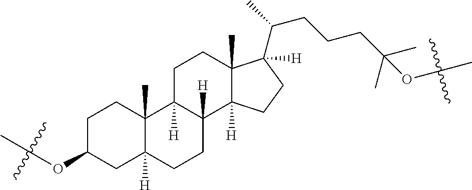 |
| 202 | 1 | 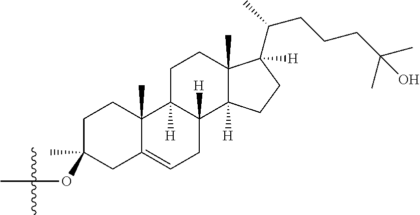 |
| 203 | 1 | 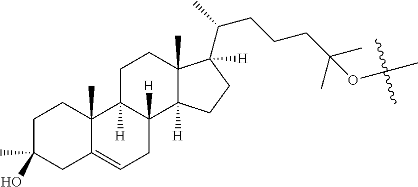 |
| 204 | 2 | 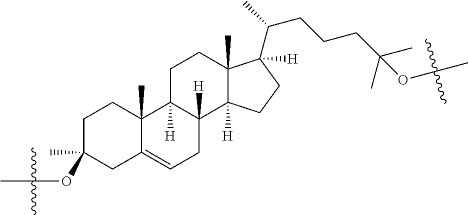 |
| 205 | 1 | 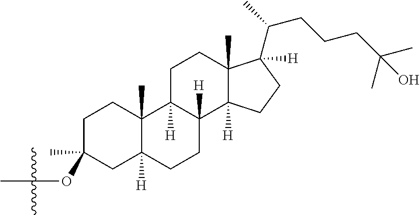 |
| 206 | 1 | 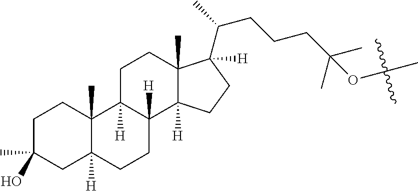 |
| 207 | 2 | 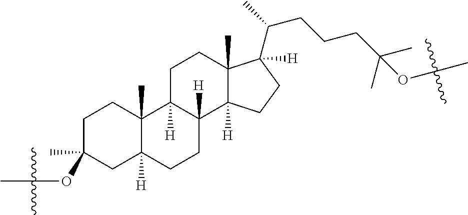 |
| 208 | 1 | 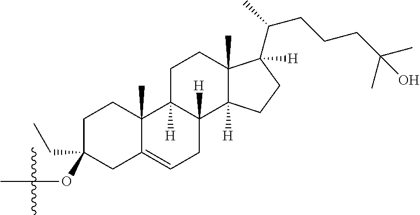 |
| 209 | 1 | 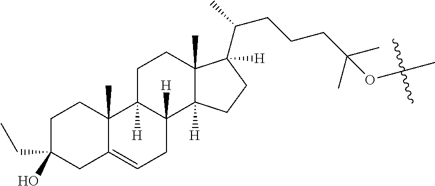 |
| 210 | 2 | 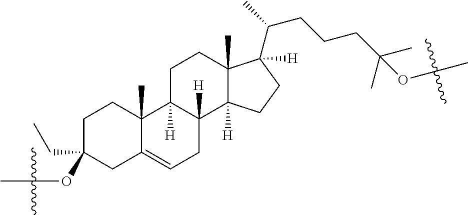 |
| 211 | 1 | 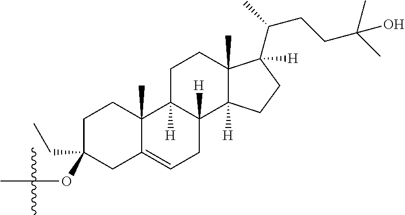 |
| 212 | 1 | 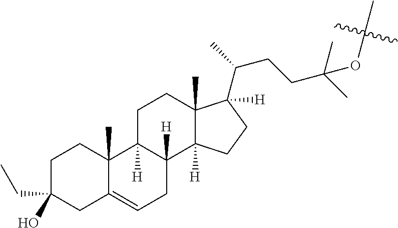 |
| 213 | 2 | 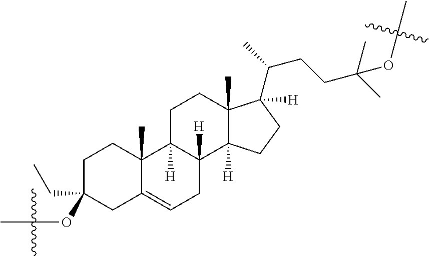 |

In some embodiments, the compound is selected from any of the compounds depicted in in Table 5, pharmaceutically acceptable salts thereof, deuterated variants thereof or combinations thereof.

TABLE 5
Formula (XXXI)

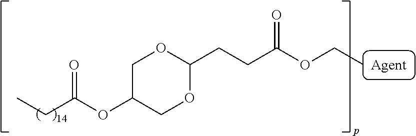

| Cpd. No. | p | 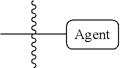 |
|---|---|---|
| 214 | 1 | 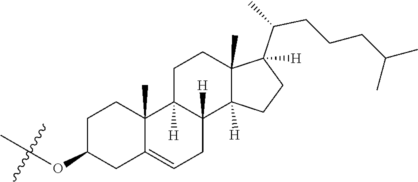 |
| 215 | 1 | 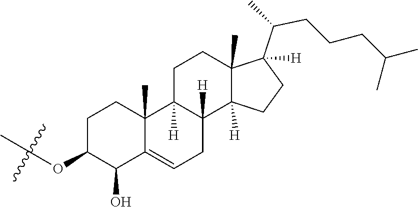 |
| 216 | 1 | 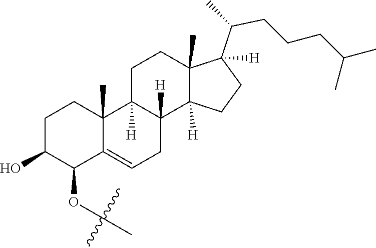 |
| 217 | 2 | 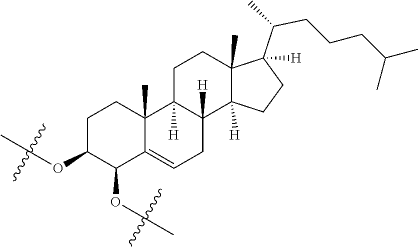 |
| 218 | 1 | 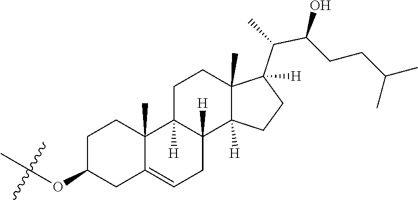 |
| 219 | 1 | 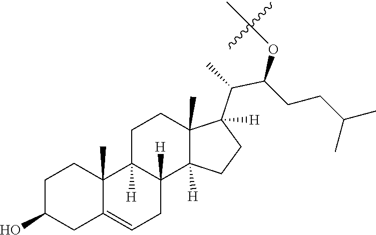 |
| 220 | 2 | 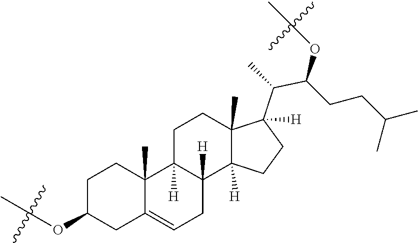 |
| 221 | 1 | 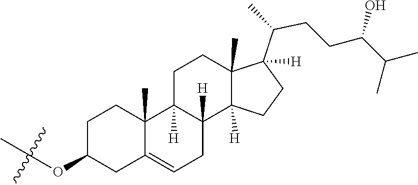 |
| 222 | 1 | 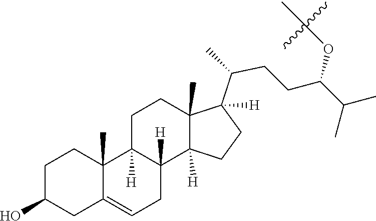 |
| 223 | 2 | 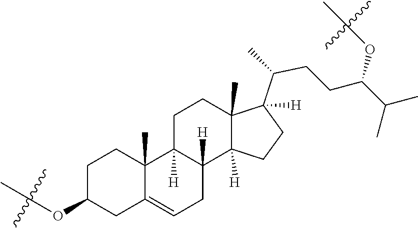 |
| 224 | 1 | 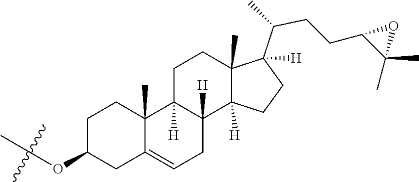 |
| 225 | 1 | 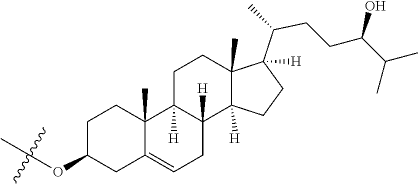 |
| 226 | 1 | 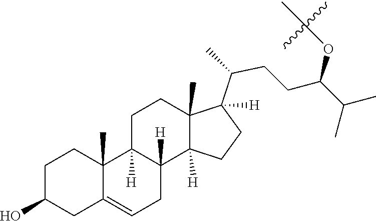 |
| 227 | 2 | 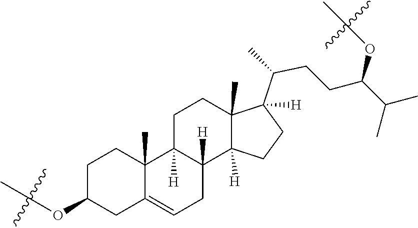 |
| 228 | 1 | 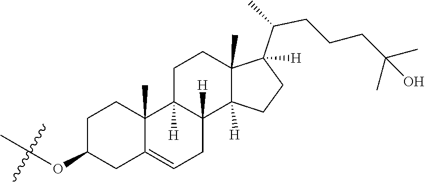 |
| 229 | 1 | 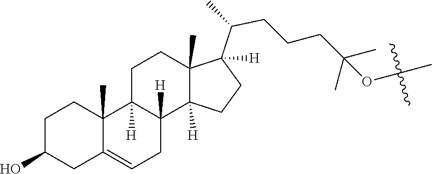 |
| 230 | 2 | 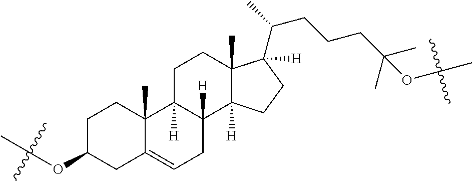 |
| 231 | 1 | 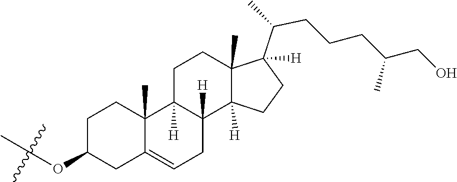 |
| 232 | 1 | 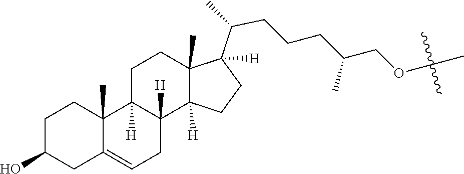 |
| 233 | 2 | 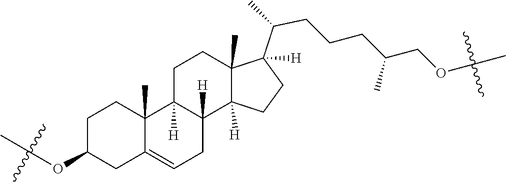 |
| 234 | 1 | 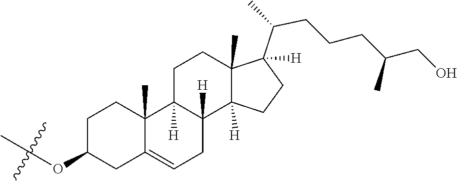 |
| 235 | 1 | 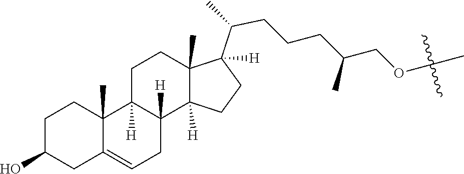 |
| 236 | 2 | 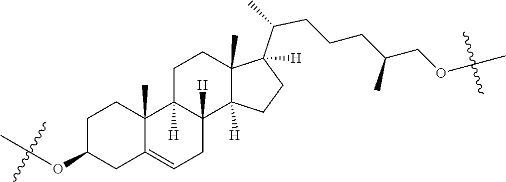 |
| 237 | 1 | 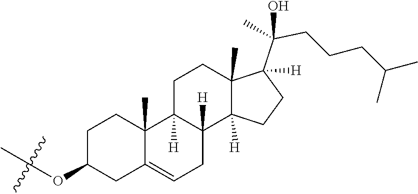 |
| 238 | 1 | 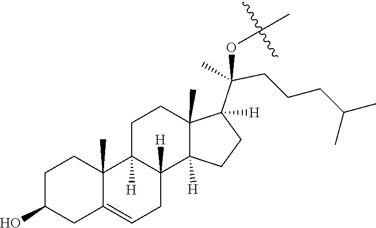 |
| 239 | 2 | 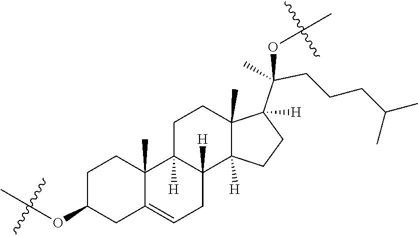 |
| 240 | 1 | 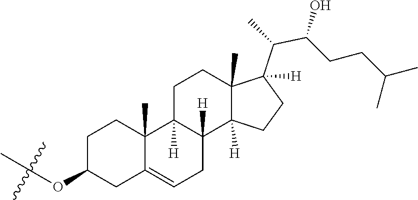 |
| 241 | 1 | 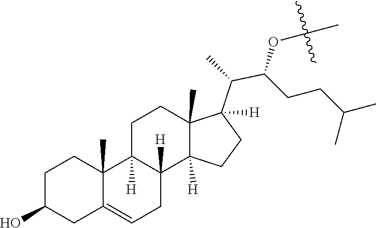 |
| 242 | 2 | 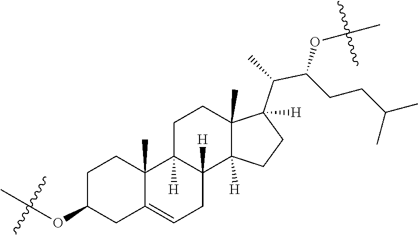 |
| 243 | 1 | 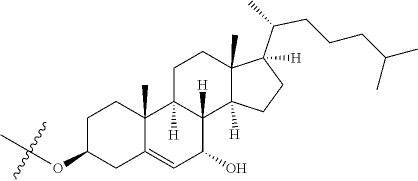 |
| 244 | 1 | 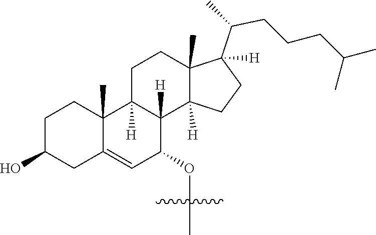 |
| 245 | 2 | 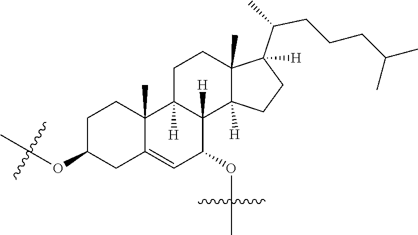 |
| 246 | 1 | 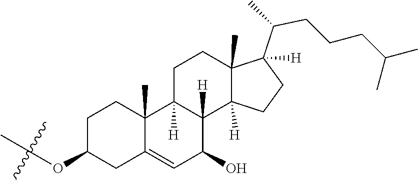 |
| 247 | 1 | 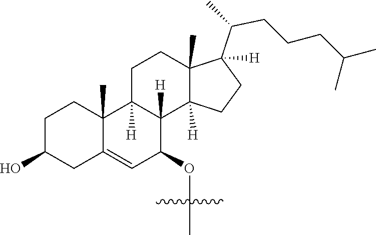 |
| 248 | 2 | 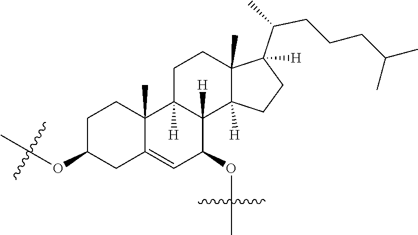 |
| 249 | 1 | 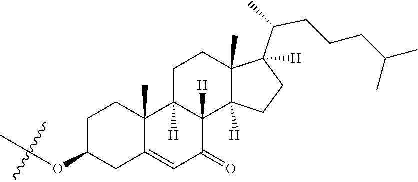 |
| 250 | 1 | 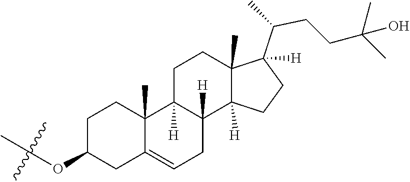 |
| 251 | 1 | 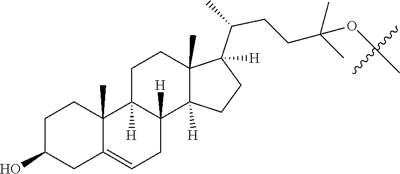 |
| 252 | 2 | 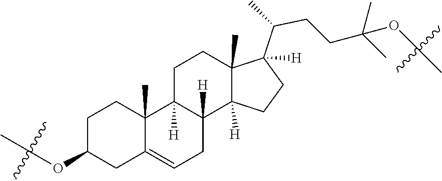 |
| 253 | 1 | 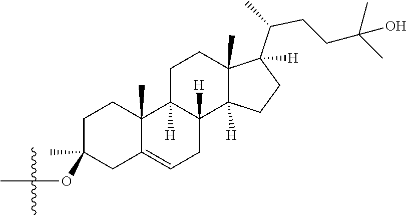 |
| 254 | 1 | 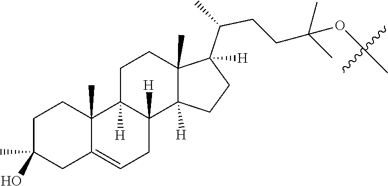 |
| 255 | 2 | 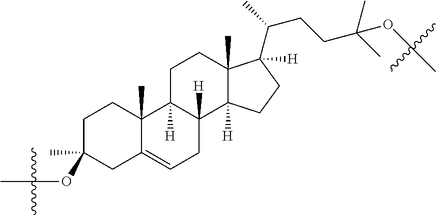 |
| 256 | 1 | 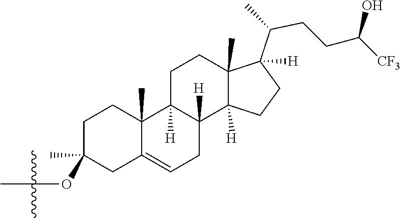 |
| 257 | 1 | 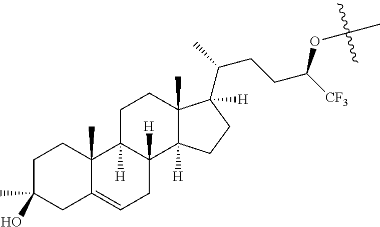 |
| 258 | 2 | 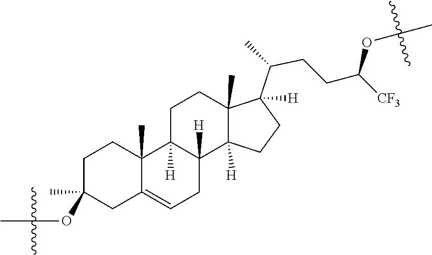 |
| 259 | 1 | 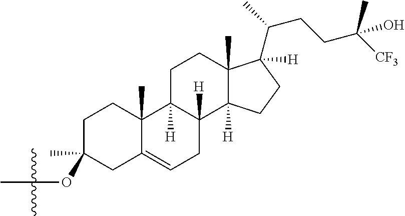 |
| 260 | 1 | 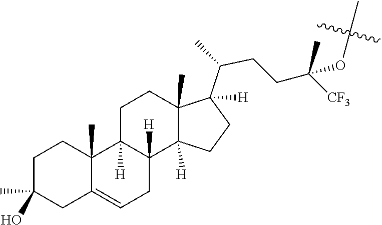 |
| 261 | 2 | 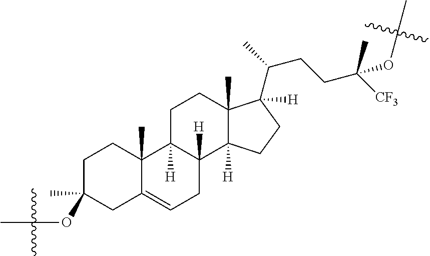 |
| 262 | 1 | 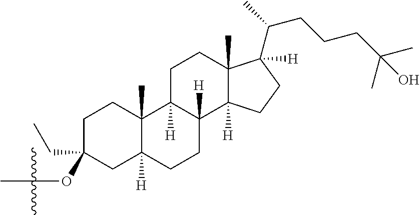 |
| 263 | 1 | 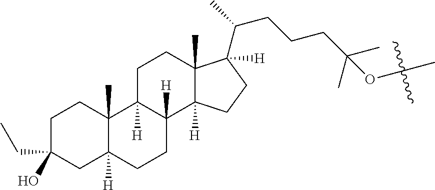 |
| 264 | 2 | 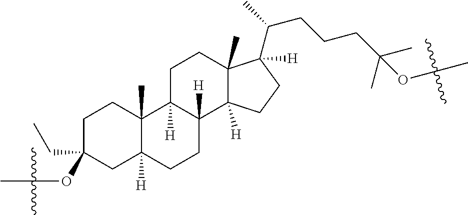 |
| 265 | 1 | 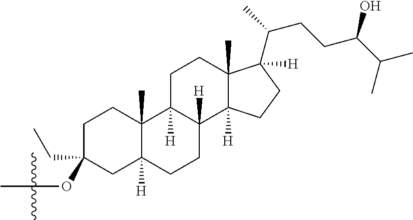 |
| 266 | 1 | 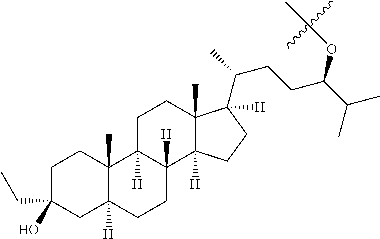 |
| 267 | 2 | 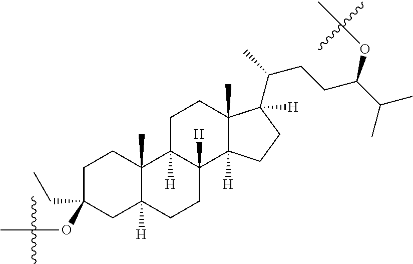 |
| 268 | 1 | 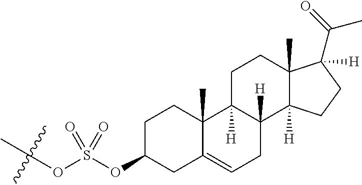 |
| 269 | 1 | 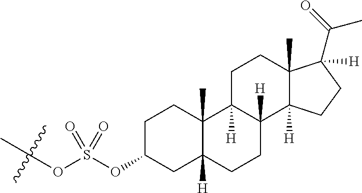 |
| 270 | 1 | 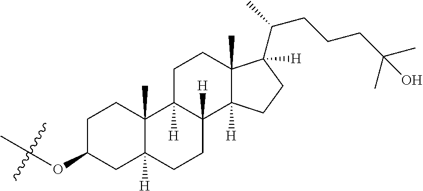 |
| 271 | 1 | 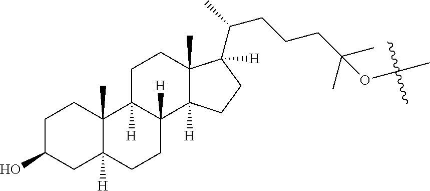 |
| 272 | 2 | 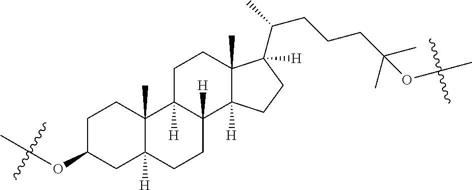 |
| 273 | 1 | 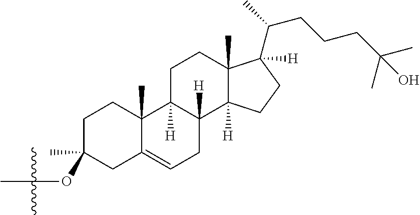 |
| 274 | 1 | 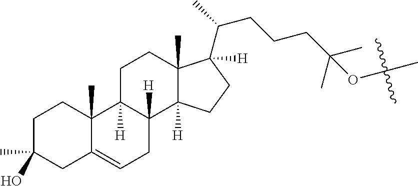 |
| 275 | 2 | 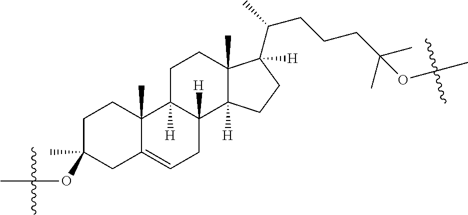 |
| 276 | 1 | 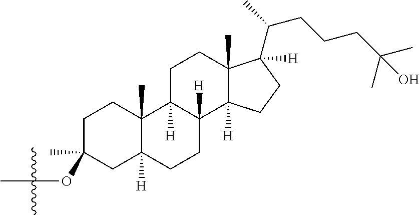 |
| 277 | 1 | 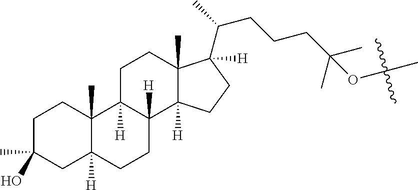 |
| 278 | 2 | 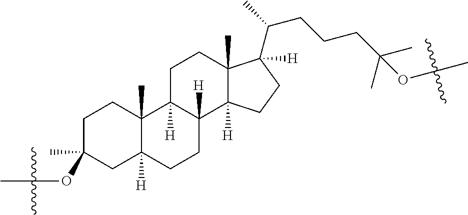 |
| 279 | 1 | 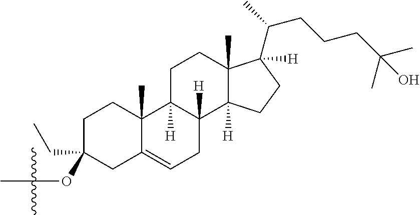 |
| 280 | 1 | 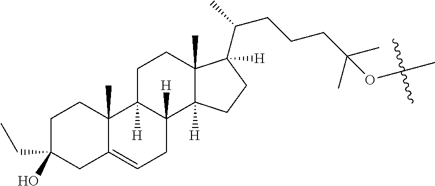 |
| 281 | 2 | 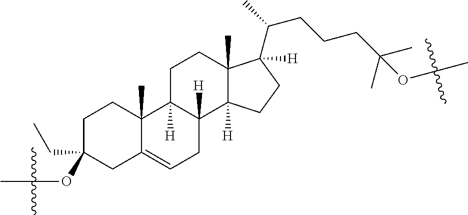 |
| 282 | 1 | 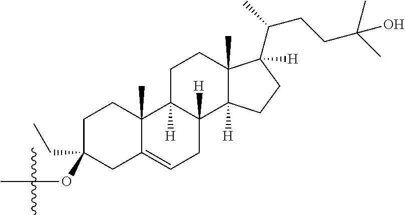 |
| 283 | 1 | 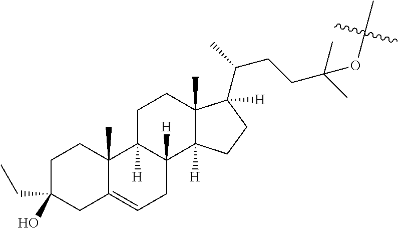 |
| 284 | 2 | 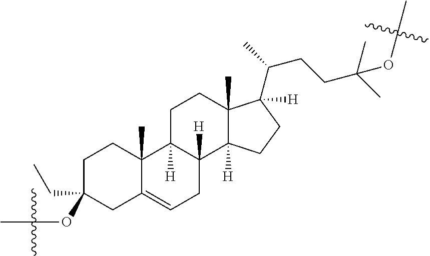 |

In some embodiments, the compound is selected from any of the compounds depicted in in Table 6, pharmaceutically acceptable salts thereof, deuterated variants thereof or combinations thereof.

TABLE 6
Formula (XXXII)

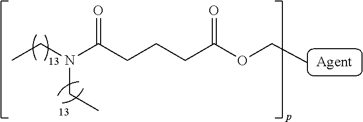

| Cpd. No. | p | 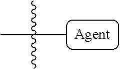 |
|---|---|---|
| 285 | 1 | 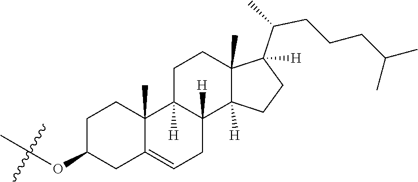 |
| 286 | 1 | 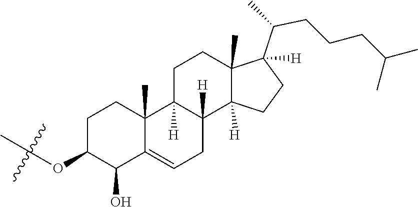 |
| 287 | 1 | 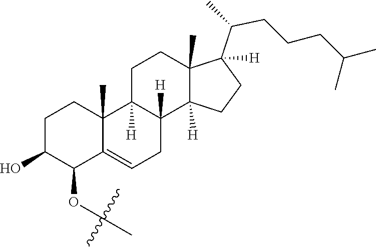 |
| 288 | 2 | 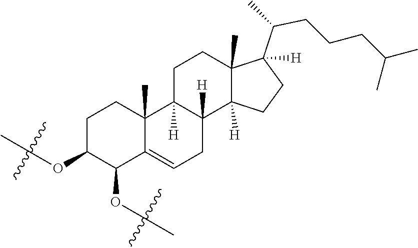 |
| 289 | 1 | 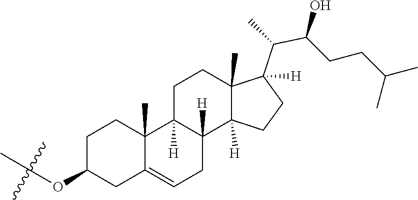 |
| 290 | 1 | 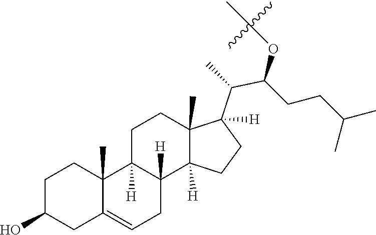 |
| 291 | 2 | 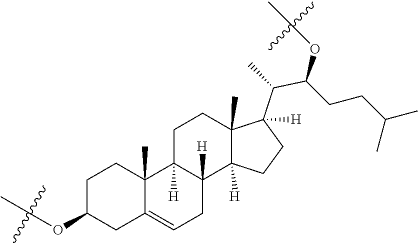 |
| 292 | 1 | 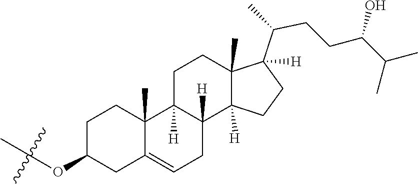 |
| 293 | 1 | 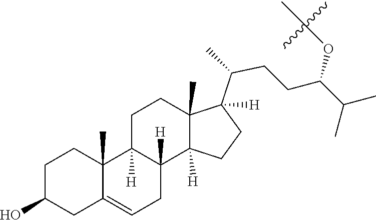 |
| 294 | 2 | 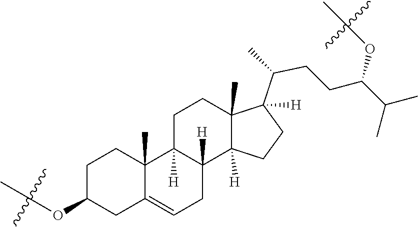 |
| 295 | 1 | 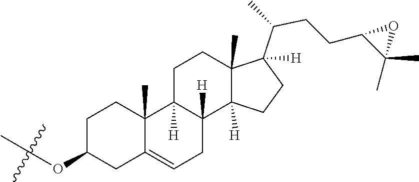 |
| 296 | 1 | 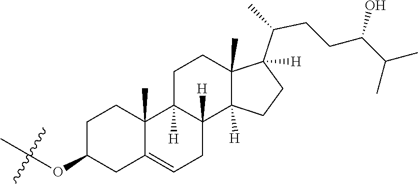 |
| 297 | 1 | 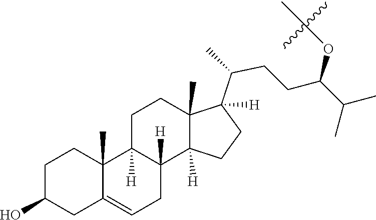 |
| 298 | 2 | 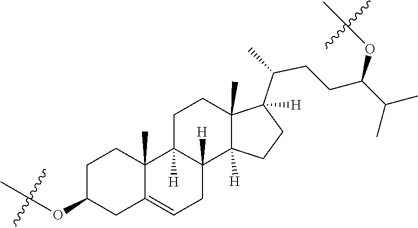 |
| 299 | 1 | 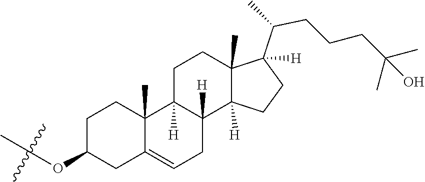 |
| 300 | 1 | 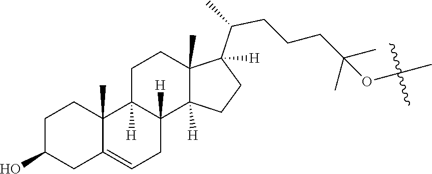 |
| 301 | 2 | 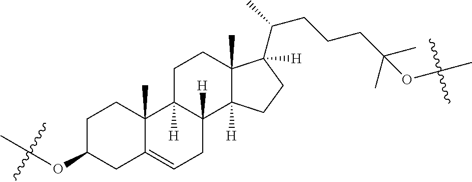 |
| 302 | 1 | 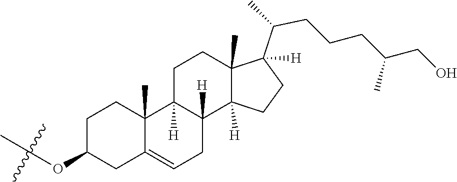 |
| 303 | 1 | 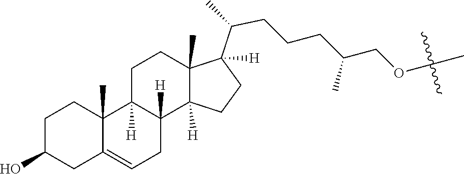 |
| 304 | 2 | 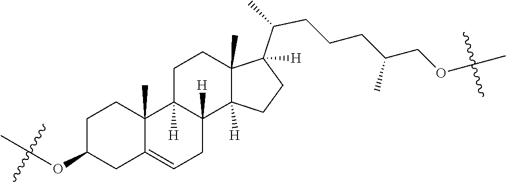 |
| 305 | 1 | 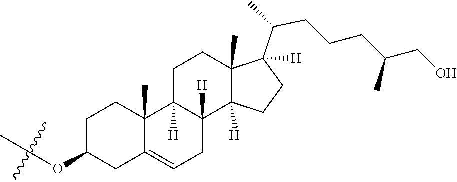 |
| 306 | 1 | 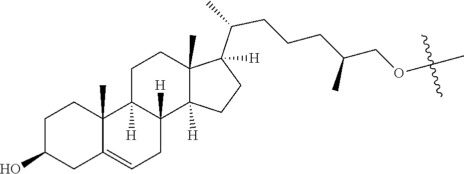 |
| 307 | 2 | 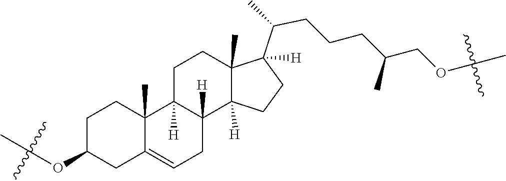 |
| 308 | 1 | 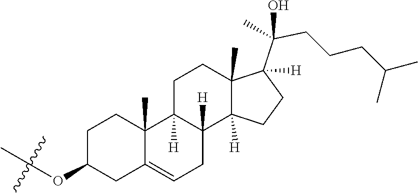 |
| 309 | 1 | 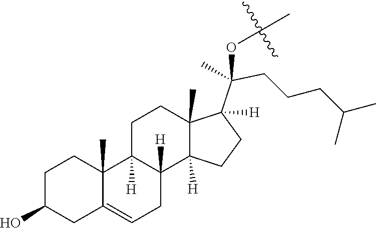 |
| 310 | 2 | 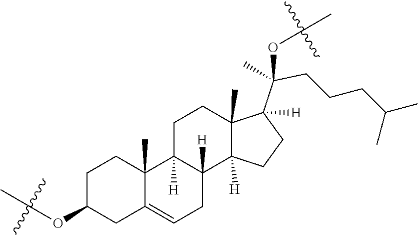 |
| 311 | 1 | 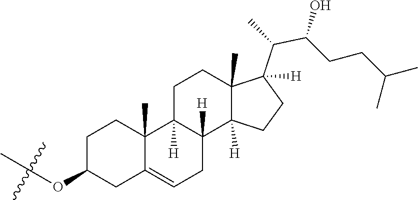 |
| 312 | 1 | 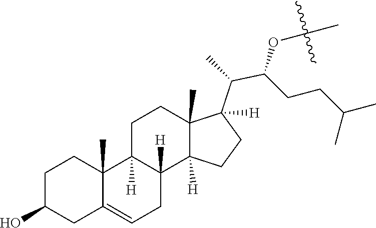 |
| 313 | 2 | 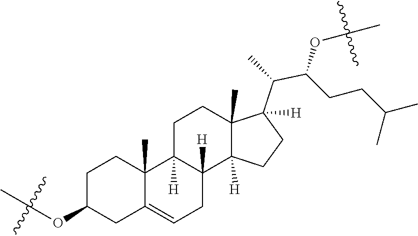 |
| 314 | 1 | 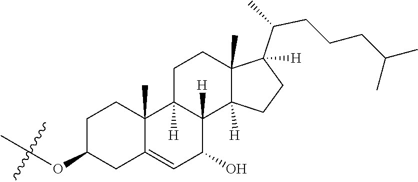 |
| 315 | 1 | 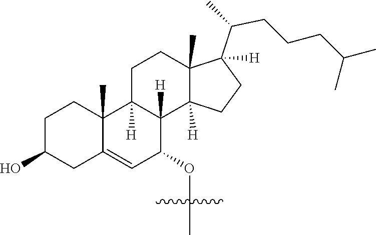 |
| 316 | 2 | 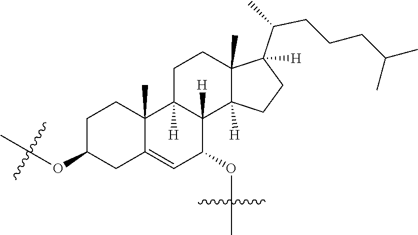 |
| 317 | 1 | 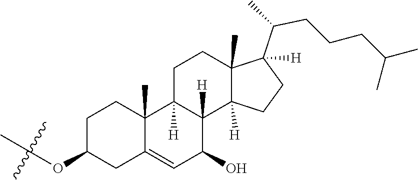 |
| 318 | 1 | 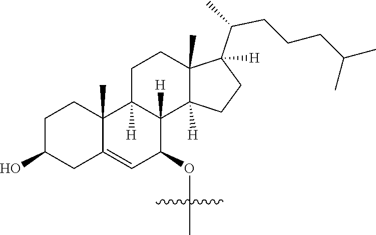 |
| 319 | 2 | 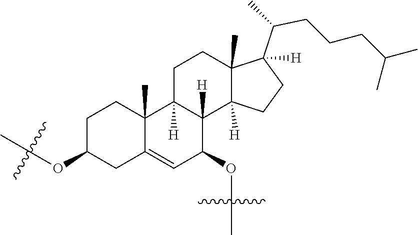 |
| 320 | 1 | 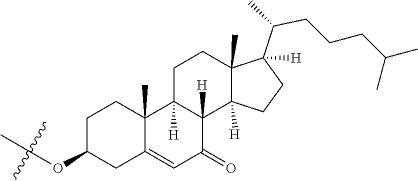 |
| 321 | 1 | 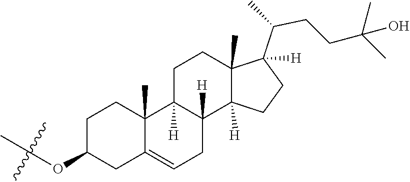 |
| 322 | 1 | 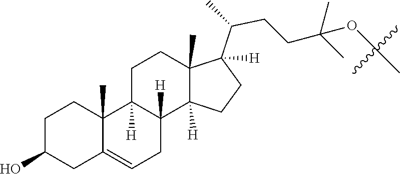 |
| 323 | 2 | 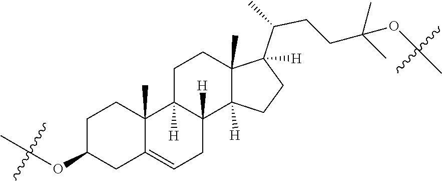 |
| 324 | 1 | 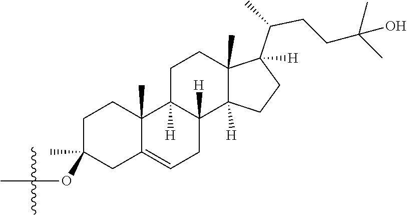 |
| 325 | 1 | 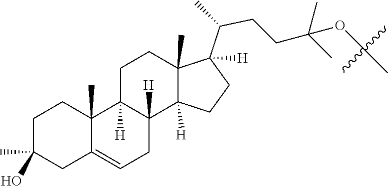 |
| 326 | 2 | 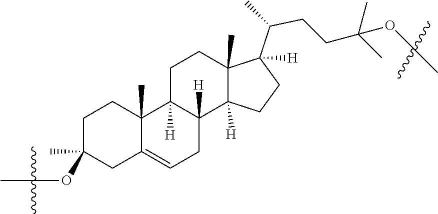 |
| 327 | 1 | 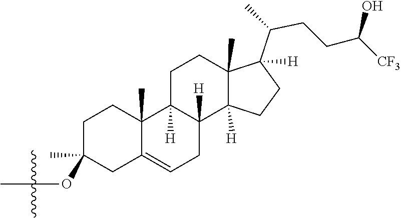 |
| 328 | 1 | 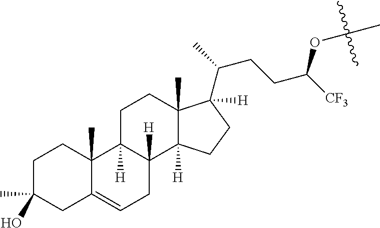 |
| 329 | 2 | 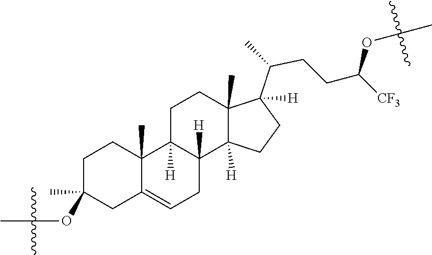 |
| 330 | 1 | 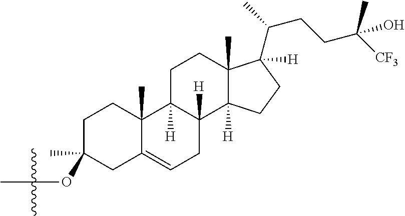 |
| 331 | 1 | 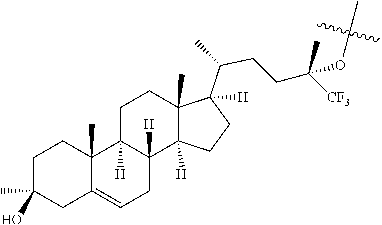 |
| 332 | 2 | 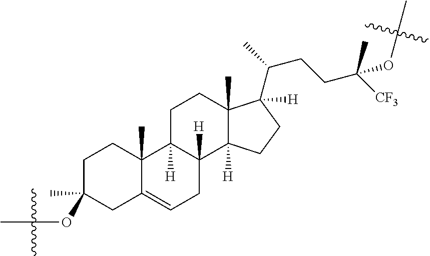 |
| 333 | 1 | 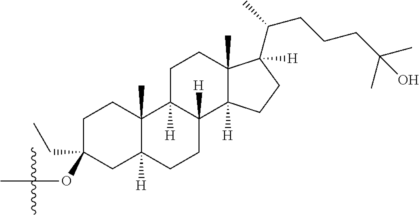 |
| 334 | 1 | 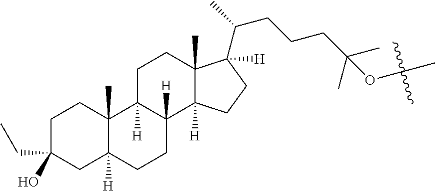 |
| 335 | 2 | 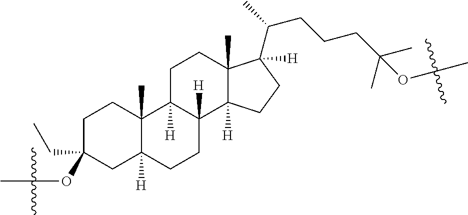 |
| 336 | 1 | 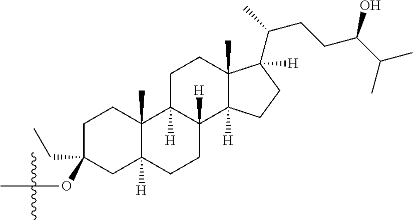 |
| 337 | 1 | 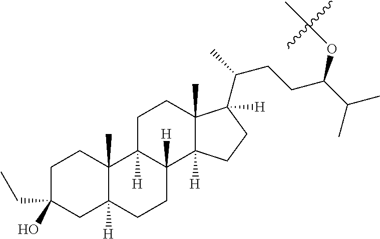 |
| 338 | 2 | 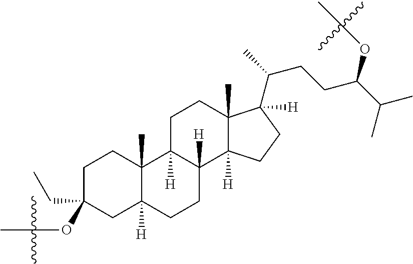 |
| 339 | 1 | 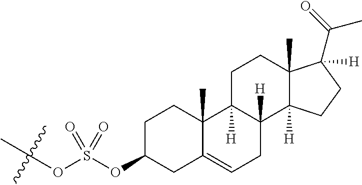 |
| 340 | 1 | 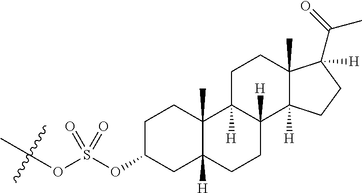 |
| 341 | 1 | 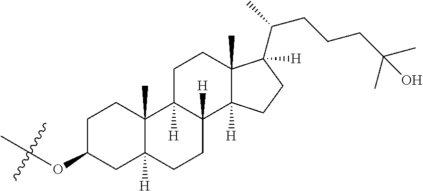 |
| 342 | 1 | 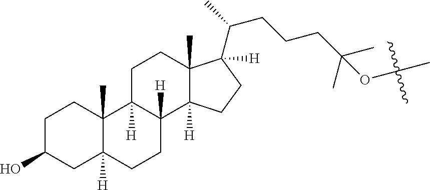 |
| 343 | 2 | 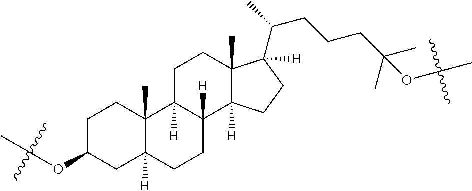 |
| 344 | 1 | 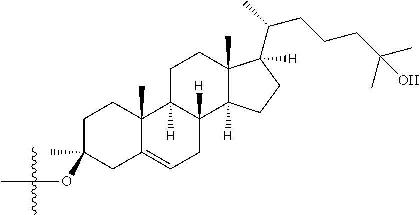 |
| 345 | 1 | 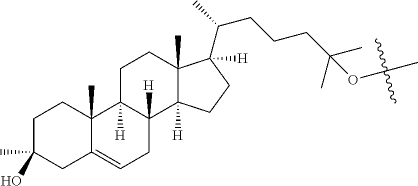 |
| 346 | 2 | 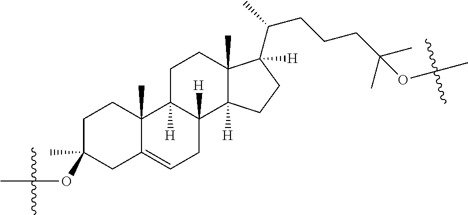 |
| 347 | 1 | 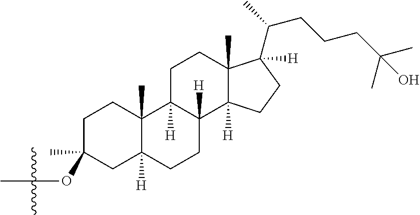 |
| 348 | 1 | 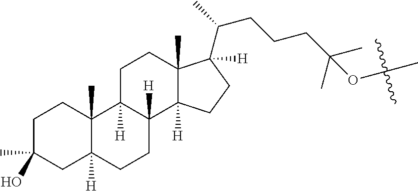 |
| 349 | 2 | 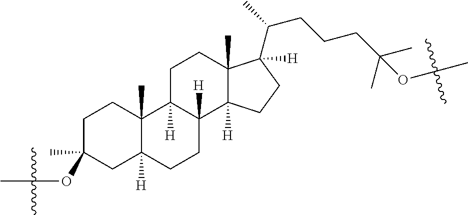 |
| 350 | 1 | 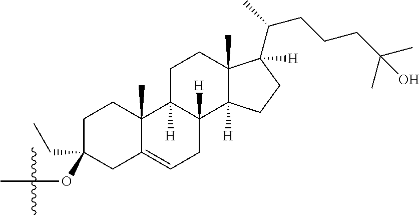 |
| 351 | 1 | 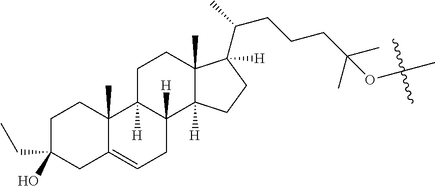 |
| 352 | 2 | 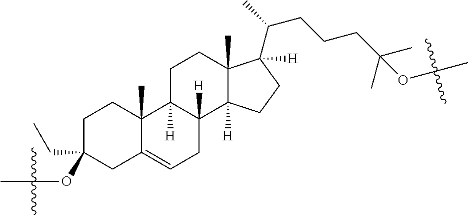 |
| 353 | 1 | 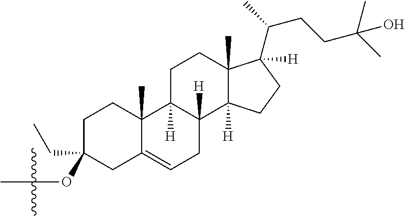 |
| 354 | 1 | 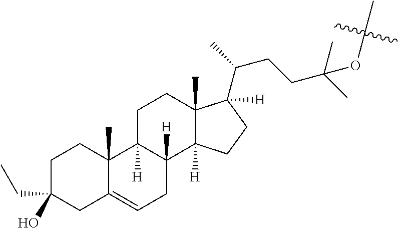 |
| 355 | 2 | 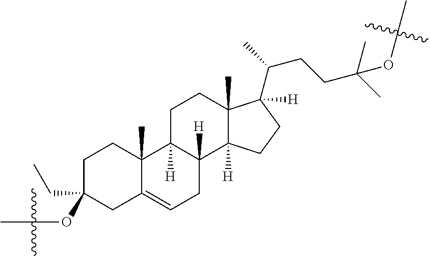 |

In some embodiments, the compound is selected from any of the compounds depicted in in Table 7, pharmaceutically acceptable salts thereof, deuterated variants thereof or combinations thereof.

TABLE 7
Formula (XXXIII)

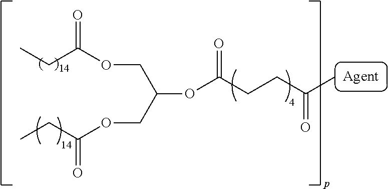

| Cpd. No. | p | 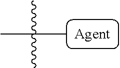 |
|---|---|---|
| 356 | 1 | 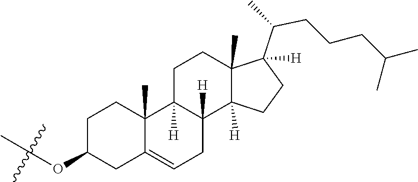 |
| 357 | 1 | 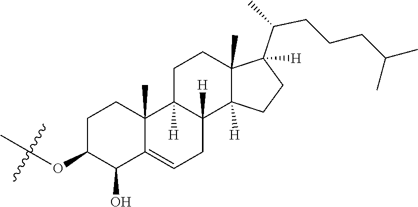 |
| 358 | 1 | 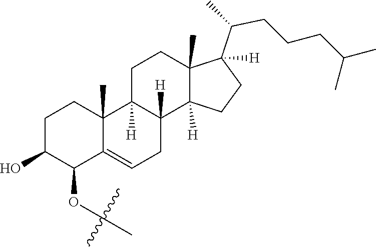 |
| 359 | 2 | 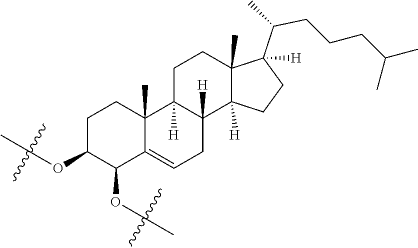 |
| 360 | 1 | 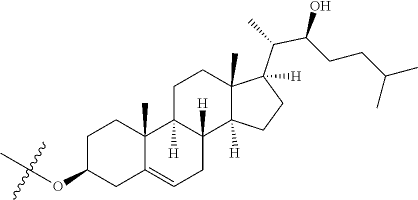 |
| 361 | 1 | 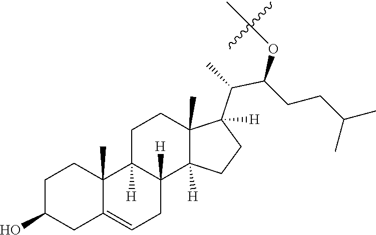 |
| 362 | 2 | 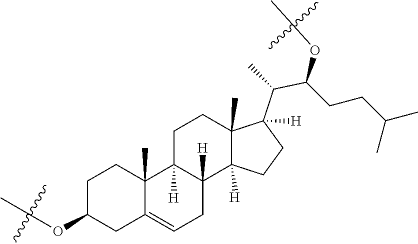 |
| 363 | 1 | 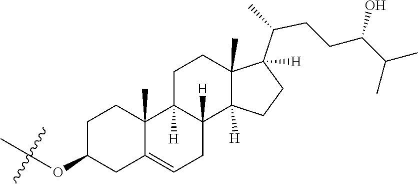 |
| 364 | 1 | 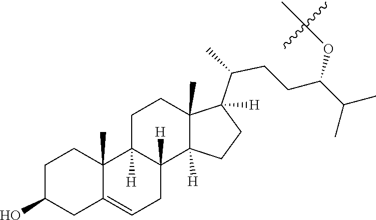 |
| 365 | 2 | 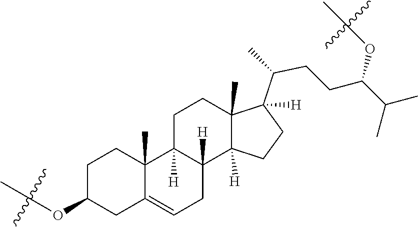 |
| 366 | 1 | 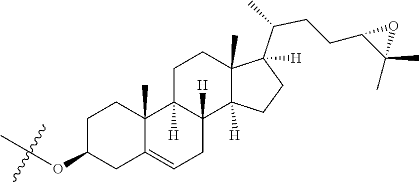 |
| 367 | 1 | 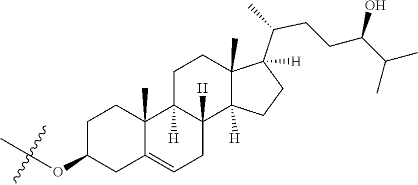 |
| 368 | 1 | 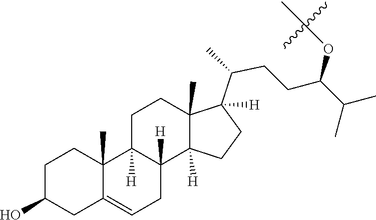 |
| 369 | 2 | 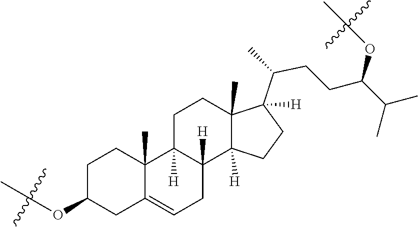 |
| 370 | 1 | 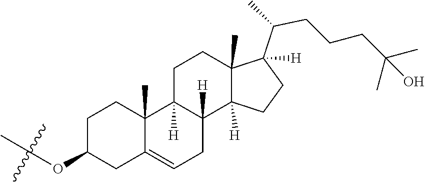 |
| 371 | 1 | 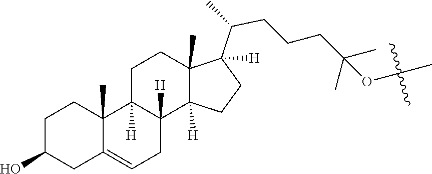 |
| 372 | 2 | 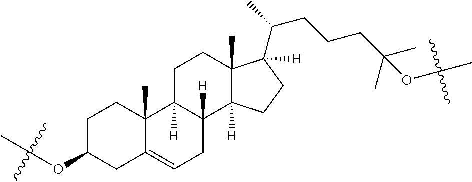 |
| 373 | 1 | 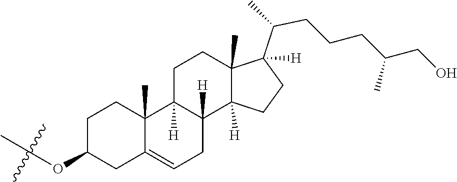 |
| 374 | 1 | 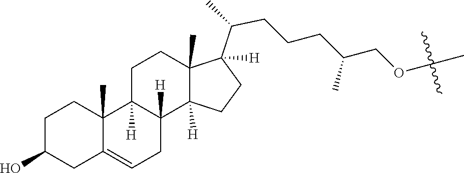 |
| 375 | 2 | 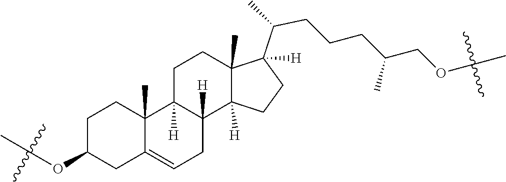 |
| 376 | 1 | 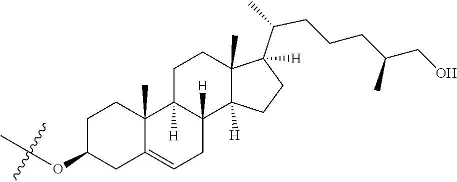 |
| 377 | 1 | 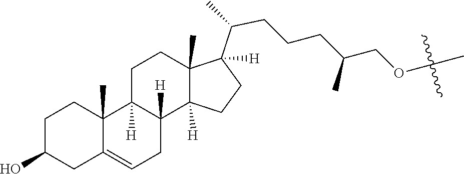 |
| 378 | 2 | 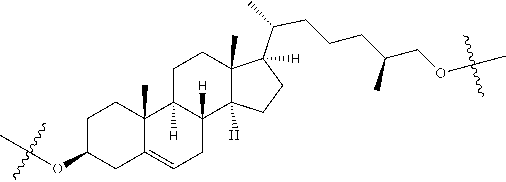 |
| 379 | 1 | 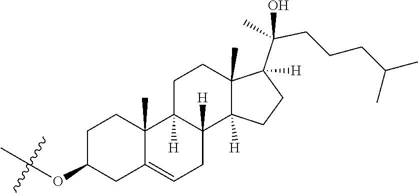 |
| 380 | 1 | 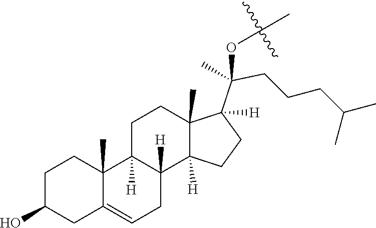 |
| 381 | 2 | 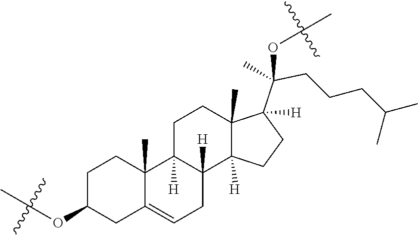 |
| 382 | 1 | 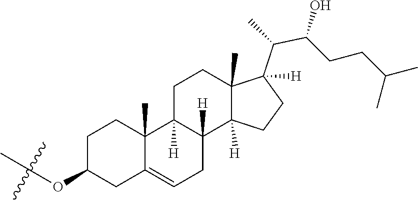 |
| 383 | 1 | 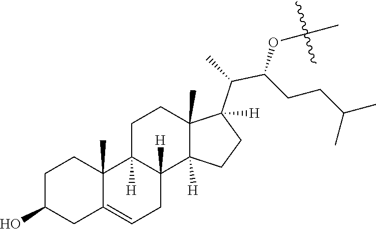 |
| 384 | 2 | 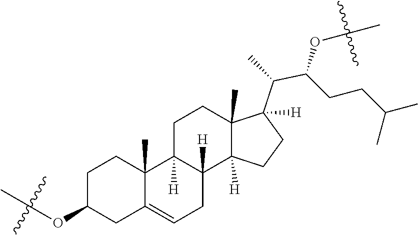 |
| 385 | 1 | 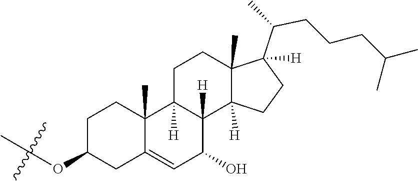 |
| 386 | 1 | 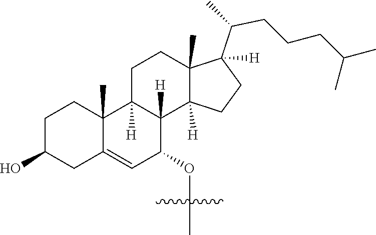 |
| 387 | 2 | 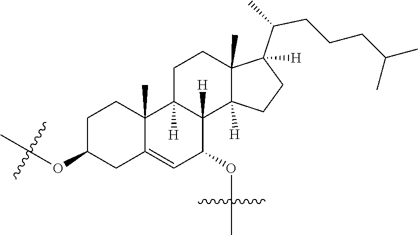 |
| 388 | 1 | 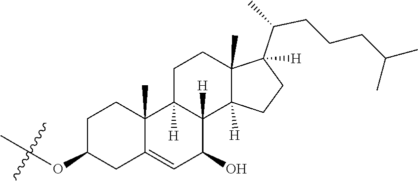 |
| 389 | 1 | 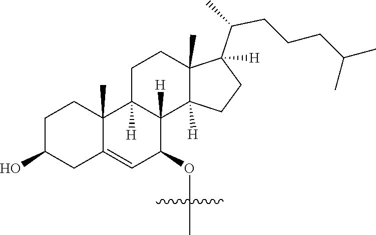 |
| 390 | 2 | 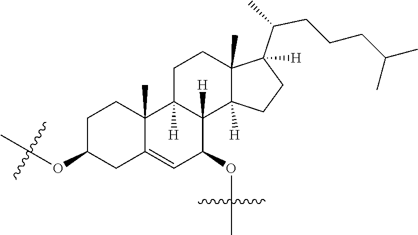 |
| 391 | 1 | 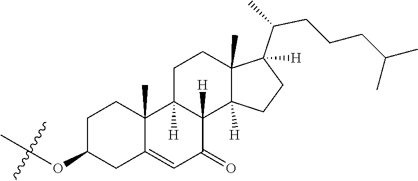 |
| 392 | 1 | 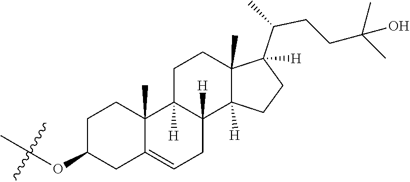 |
| 393 | 1 | 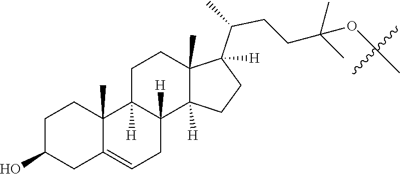 |
| 394 | 2 | 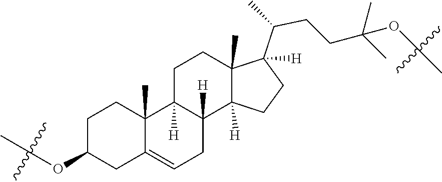 |
| 395 | 1 | 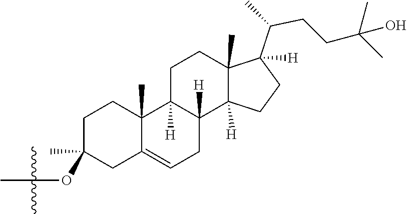 |
| 396 | 1 | 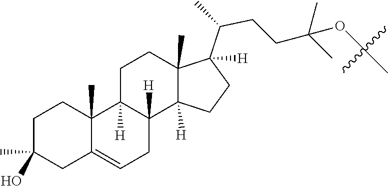 |
| 397 | 2 | 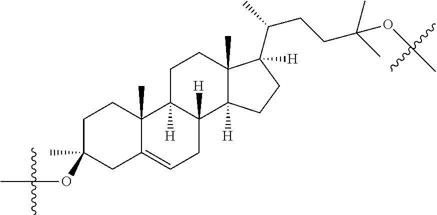 |
| 398 | 1 | 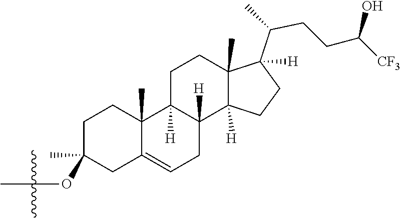 |
| 399 | 1 | 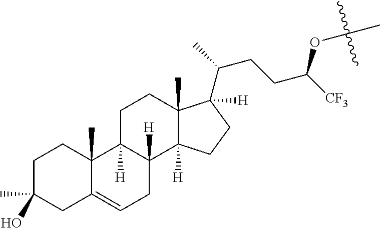 |
| 400 | 2 | 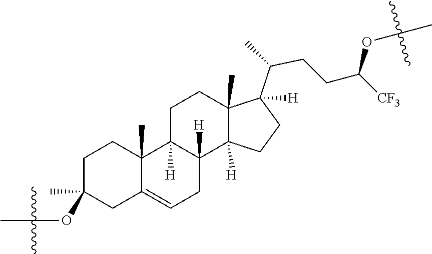 |
| 401 | 1 | 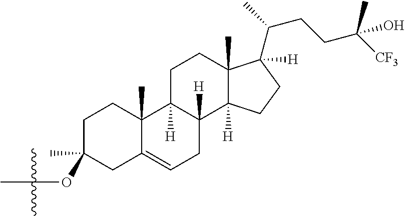 |
| 402 | 1 | 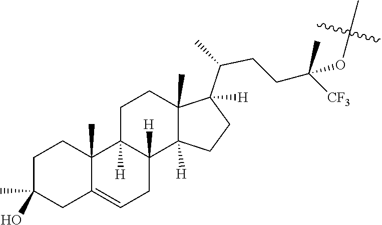 |
| 403 | 2 | 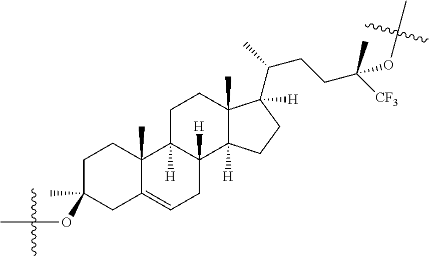 |
| 404 | 1 | 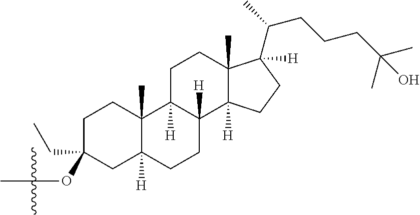 |
| 405 | 1 | 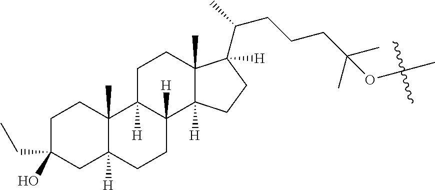 |
| 406 | 2 | 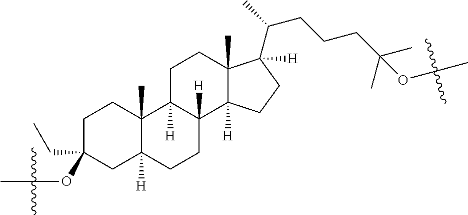 |
| 407 | 1 | 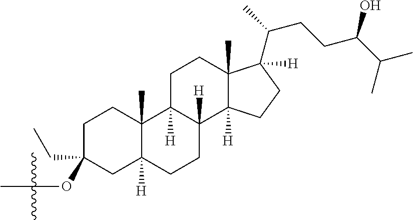 |
| 408 | 1 | 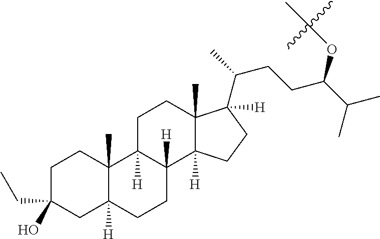 |
| 409 | 2 | 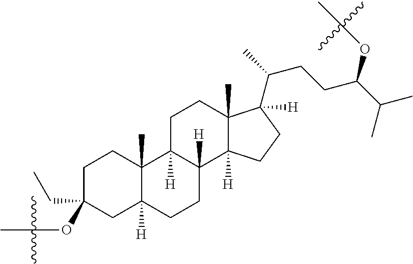 |
| 410 | 1 | 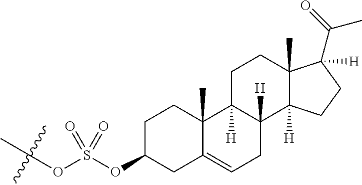 |
| 411 | 1 | 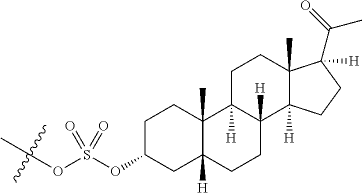 |
| 412 | 1 | 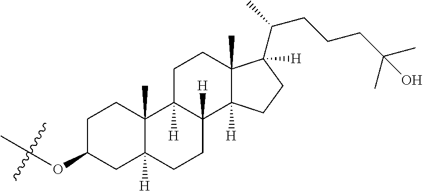 |
| 413 | 1 | 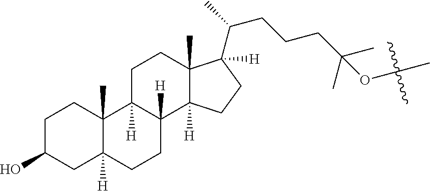 |
| 414 | 2 | 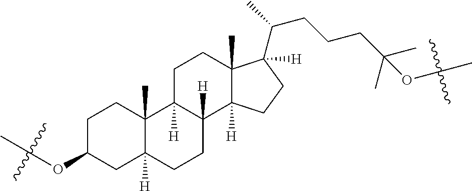 |
| 415 | 1 | 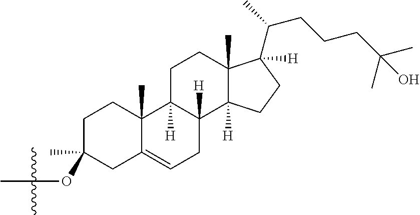 |
| 416 | 1 | 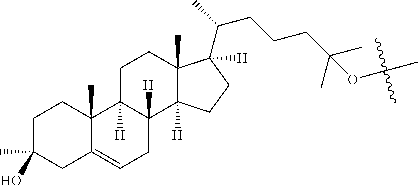 |
| 417 | 2 | 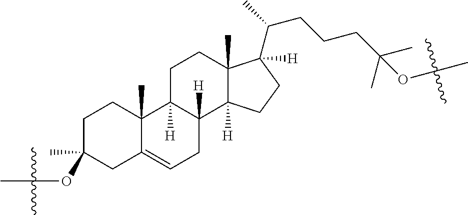 |
| 418 | 1 | 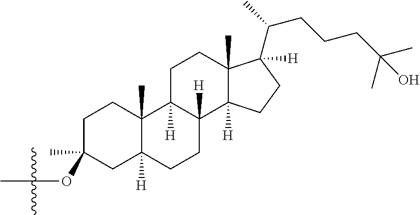 |
| 419 | 1 | 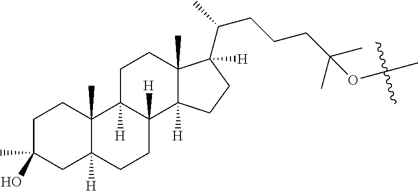 |
| 420 | 2 | 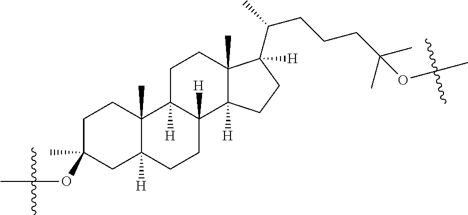 |
| 421 | 1 | 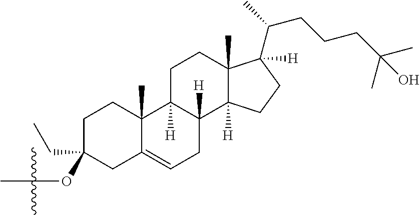 |
| 422 | 1 | 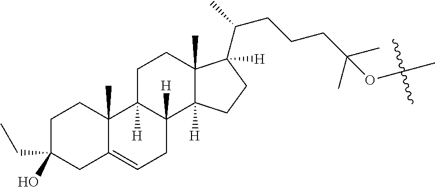 |
| 423 | 2 | 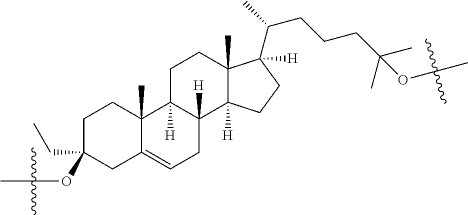 |
| 424 | 1 | 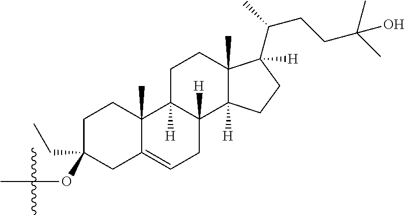 |
| 425 | 1 | 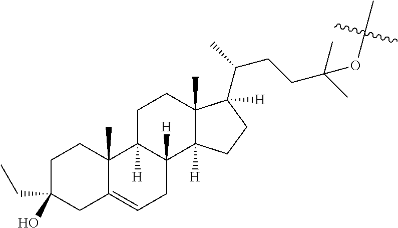 |
| 426 | 2 | 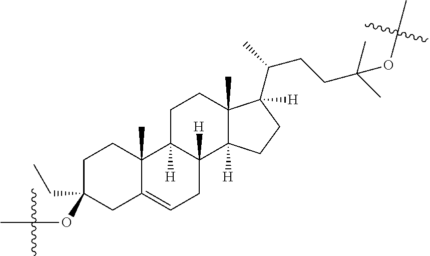 |

In some embodiments, the compound is selected from any of the compounds depicted in in Table 8, pharmaceutically acceptable salts thereof, deuterated variants thereof or combinations thereof.

TABLE 8

| | |
|---|---|
| | Formula (XXXIV) |
| 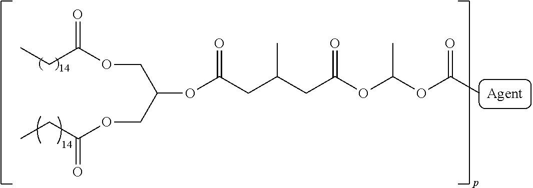 | |

| Cpd. No. | p | 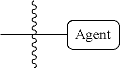 |
|---|---|---|
| 427 | 1 | 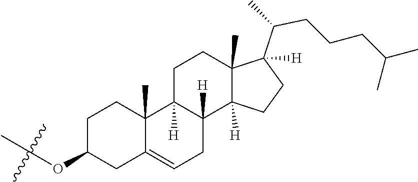 |
| 428 | 1 | 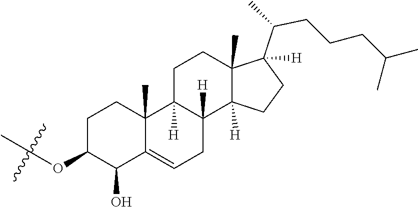 |
| 429 | 1 | 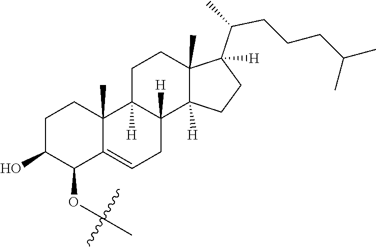 |
| 430 | 2 | 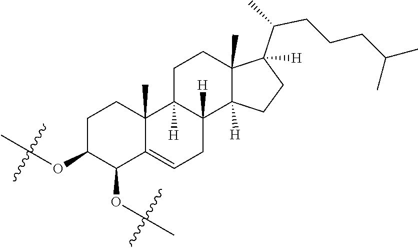 |
| 431 | 1 | 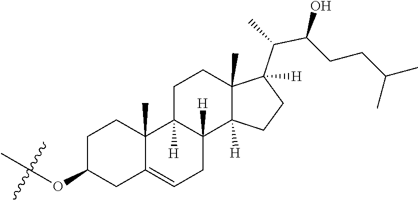 |
| 432 | 1 | 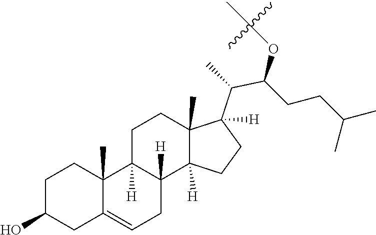 |
| 433 | 2 | 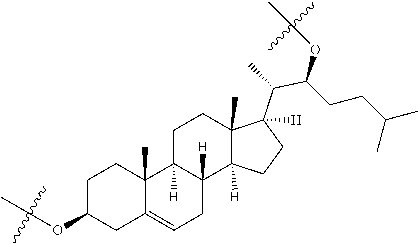 |
| 434 | 1 | 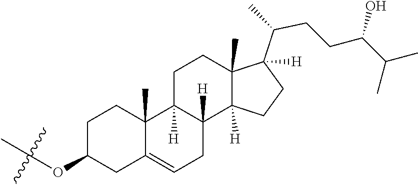 |
| 435 | 1 | 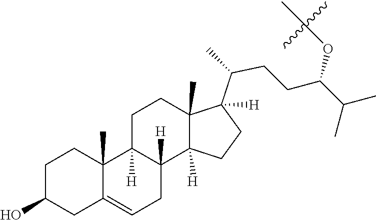 |
| 436 | 2 | 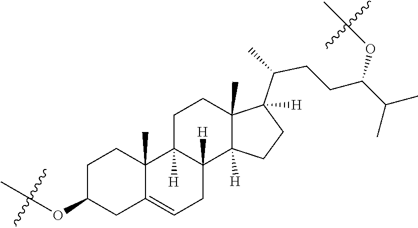 |
| 437 | 1 | 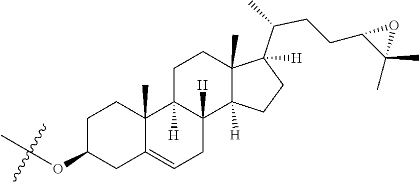 |
| 438 | 1 | 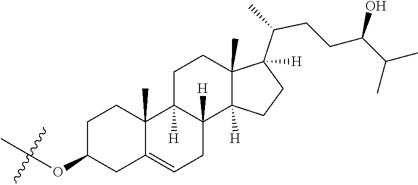 |
| 439 | 1 | 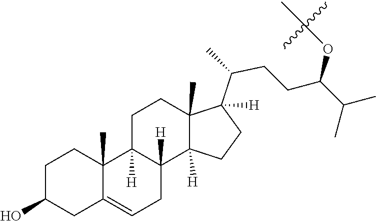 |
| 440 | 2 | 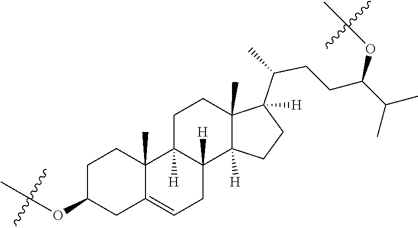 |
| 441 | 1 | 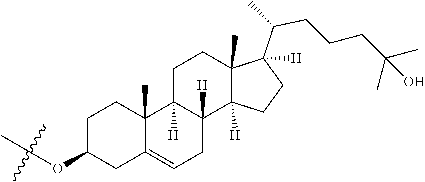 |
| 442 | 1 | 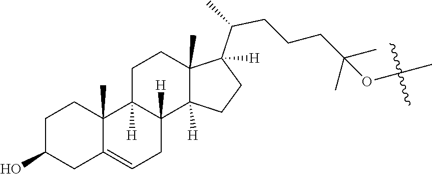 |
| 443 | 2 | 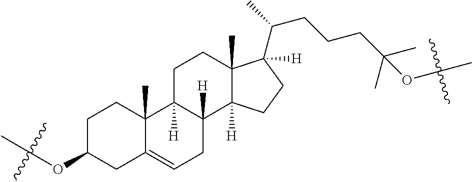 |
| 444 | 1 | 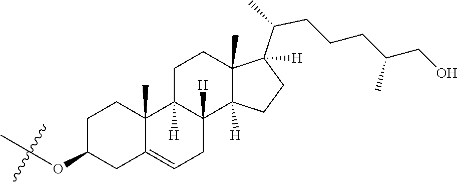 |
| 445 | 1 | 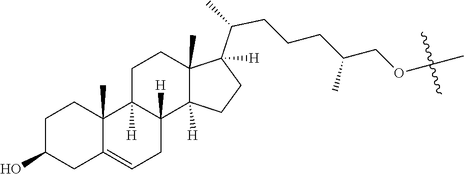 |
| 446 | 2 | 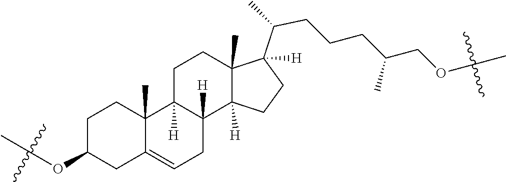 |
| 447 | 1 | 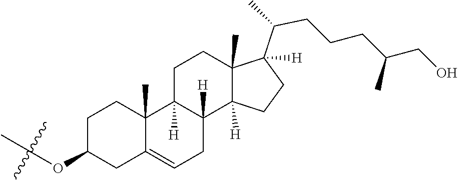 |
| 448 | 1 | 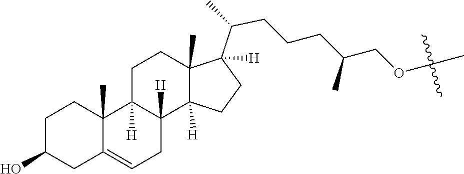 |
| 449 | 2 | 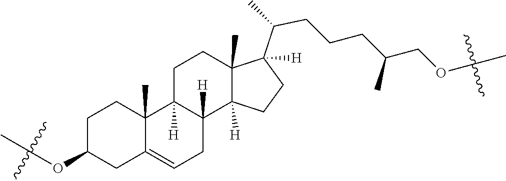 |
| 450 | 1 | 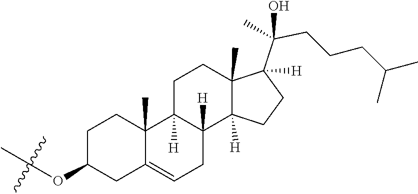 |
| 451 | 1 | 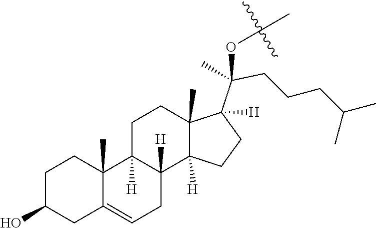 |
| 452 | 2 | 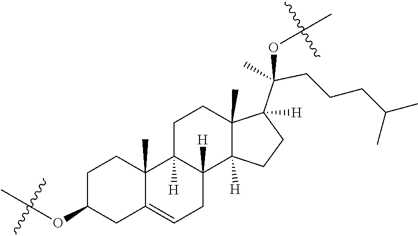 |
| 453 | 1 | 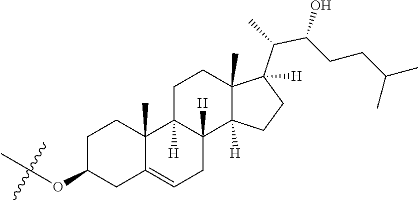 |
| 454 | 1 | 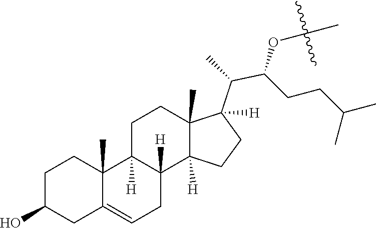 |
| 455 | 2 | 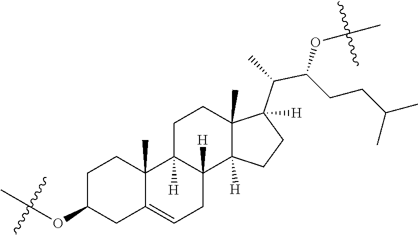 |
| 456 | 1 | 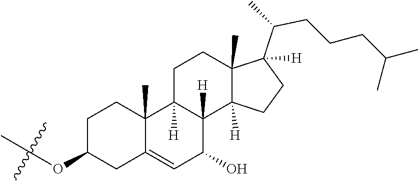 |
| 457 | 1 | 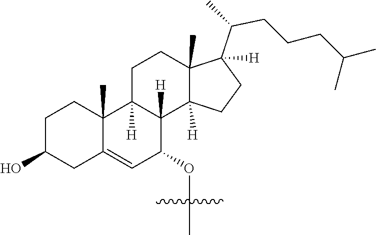 |
| 458 | 2 | 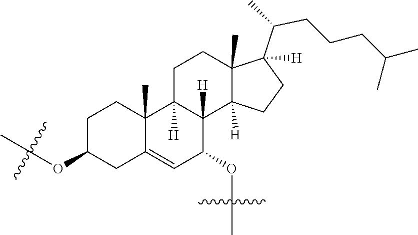 |
| 459 | 1 | 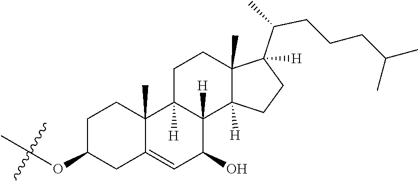 |
| 460 | 1 | 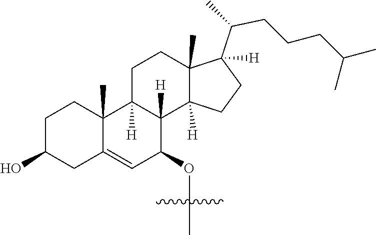 |
| 461 | 2 | 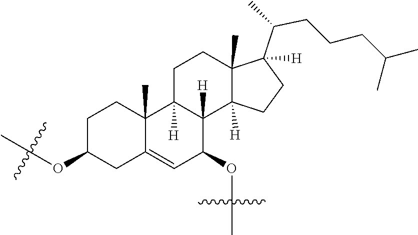 |
| 462 | 1 | 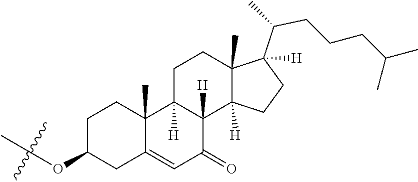 |
| 463 | 1 | 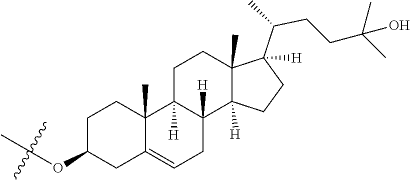 |
| 464 | 1 | 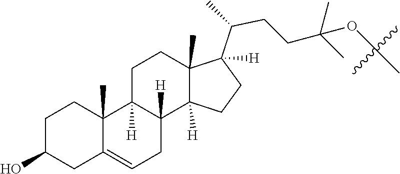 |
| 465 | 2 | 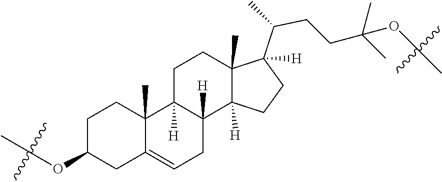 |
| 466 | 1 | 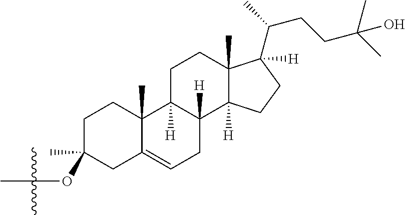 |
| 467 | 1 | 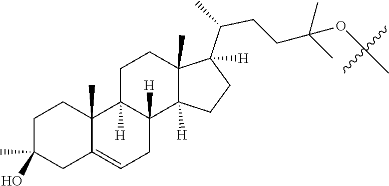 |
| 468 | 2 | 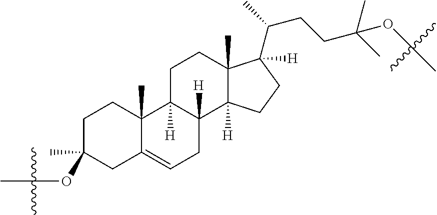 |
| 469 | 1 | 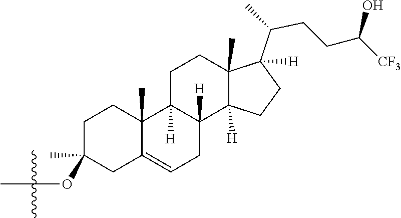 |
| 470 | 1 | 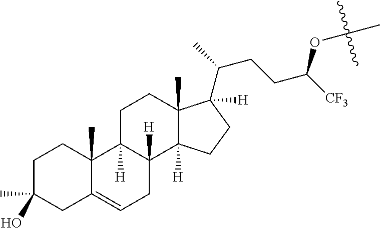 |
| 471 | 2 | 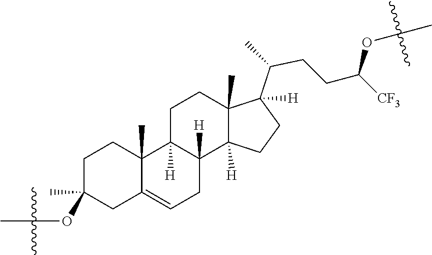 |
| 472 | 1 | 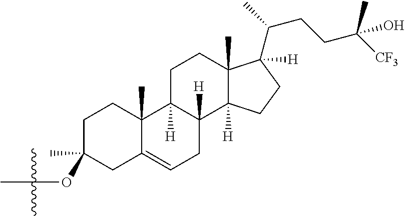 |
| 473 | 1 | 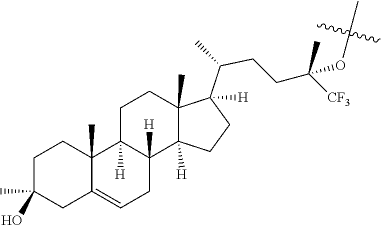 |
| 474 | 2 | 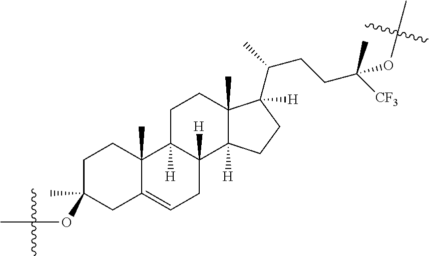 |
| 475 | 1 | 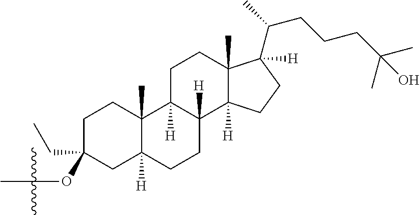 |
| 476 | 1 | 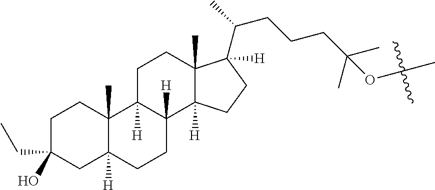 |
| 477 | 2 | 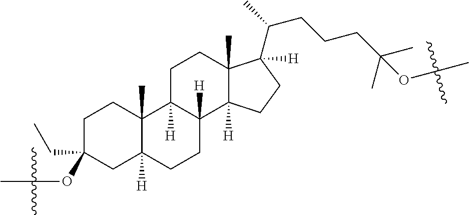 |
| 478 | 1 | 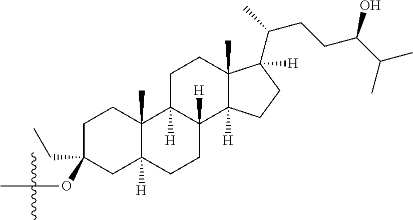 |
| 479 | 1 | 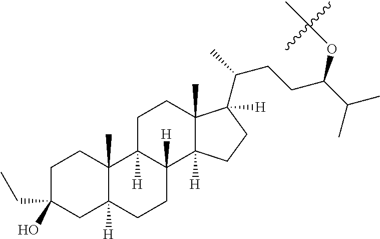 |
| 480 | 2 | 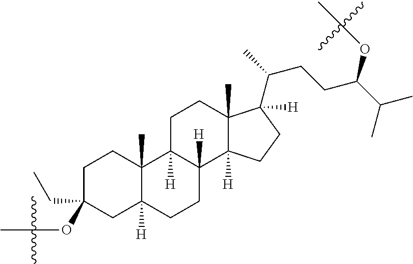 |
| 481 | 1 | 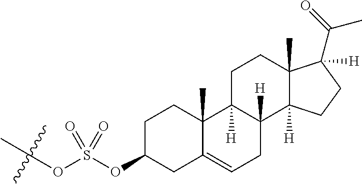 |
| 482 | 1 | 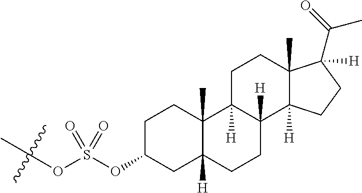 |
| 483 | 1 | 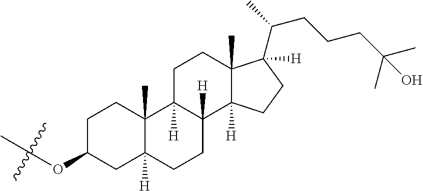 |
| 484 | 1 | 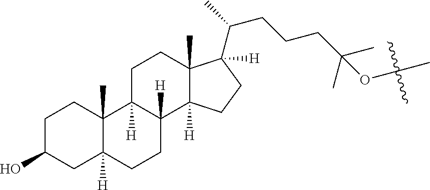 |
| 485 | 2 | 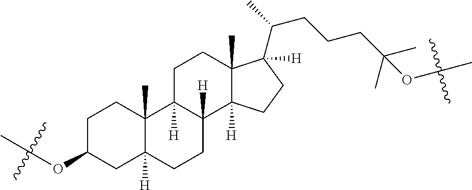 |
| 486 | 1 | 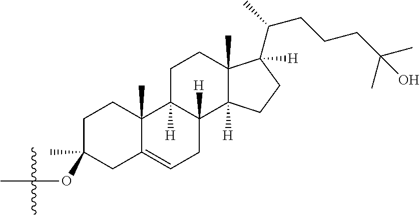 |
| 487 | 1 | 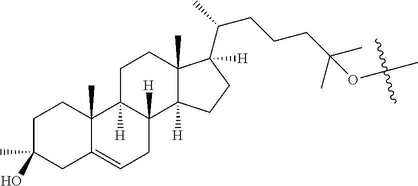 |
| 488 | 2 | 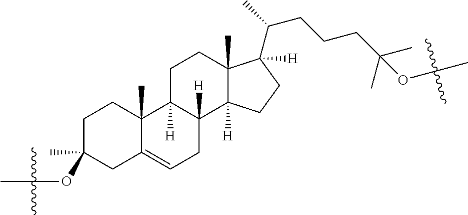 |
| 489 | 1 | 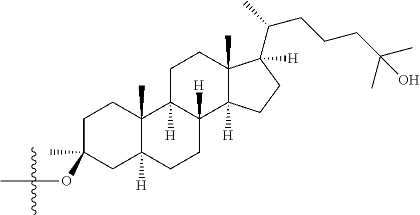 |
| 490 | 1 | 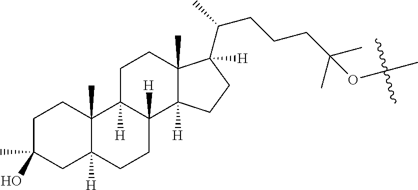 |
| 491 | 2 | 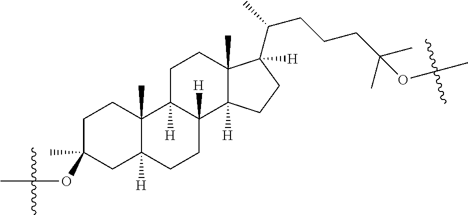 |
| 492 | 1 | 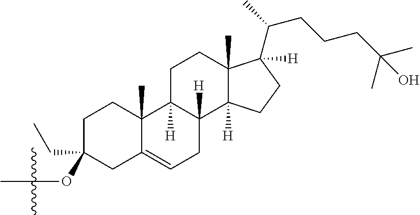 |
| 493 | 1 | 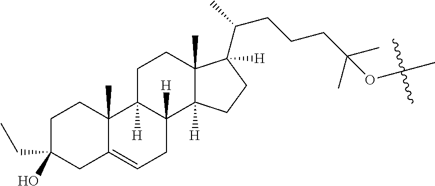 |
| 494 | 2 | 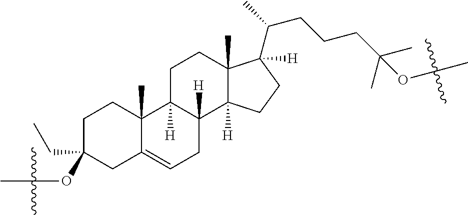 |
| 495 | 1 | 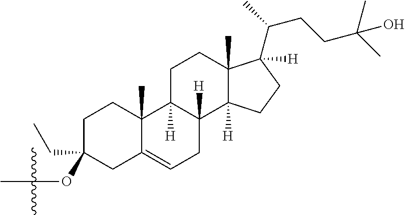 |
| 496 | 1 | 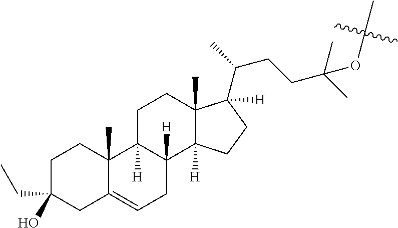 |
| 497 | 2 | 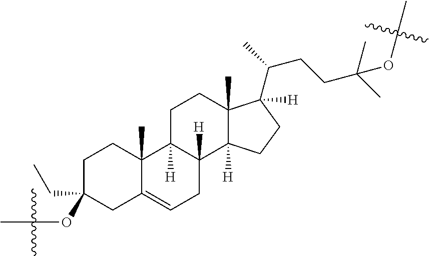 |

In some embodiments, the compound is selected from any of the compounds depicted in in Table 9, pharmaceutically acceptable salts thereof, deuterated variants thereof or combinations thereof.

TABLE 9

| | |
|---|---|
| | Formula (XXXV) |
| 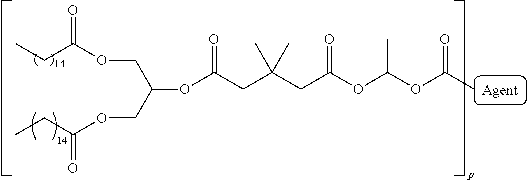 | |

| Cpd. No. | p | 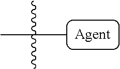 |
|---|---|---|
| 498 | 1 | 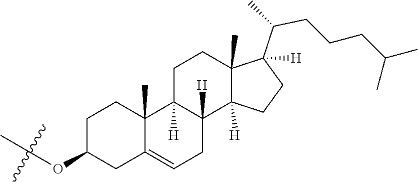 |
| 499 | 1 | 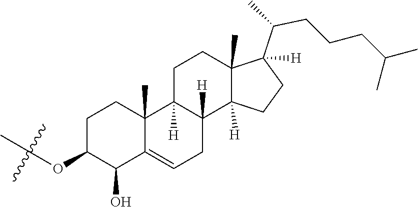 |
| 500 | 1 | 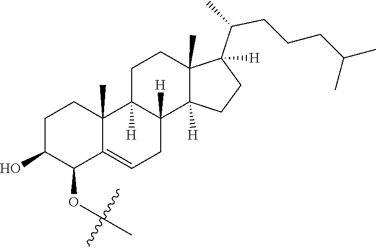 |
| 501 | 2 | 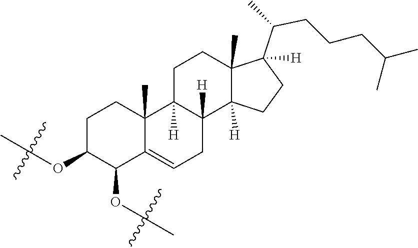 |
| 502 | 1 | 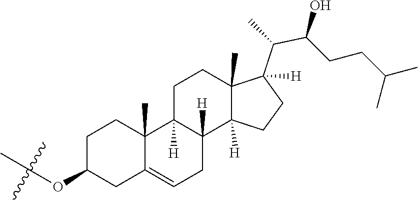 |
| 503 | 1 | 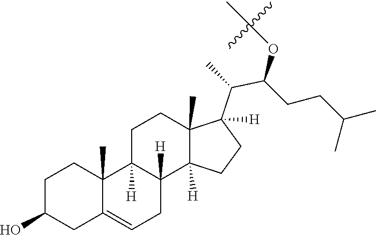 |
| 504 | 2 | 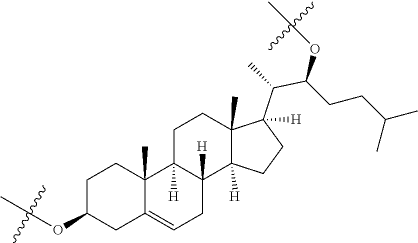 |
| 505 | 1 | 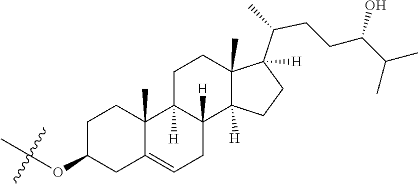 |
| 506 | 1 | 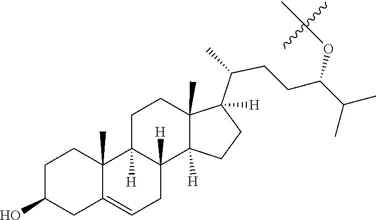 |
| 507 | 2 | 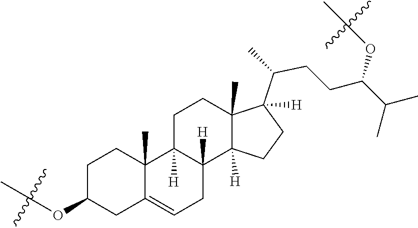 |
| 508 | 1 | 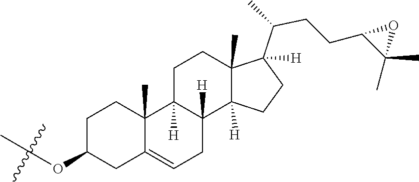 |
| 509 | 1 | 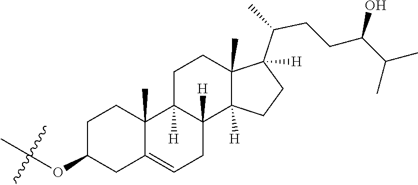 |
| 510 | 1 | 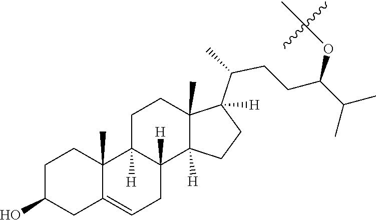 |
| 511 | 2 | 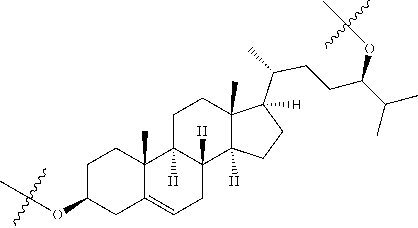 |
| 512 | 1 | 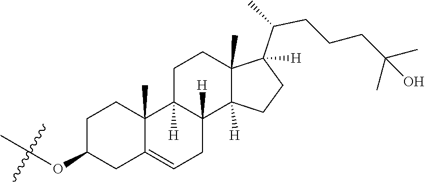 |
| 513 | 1 | 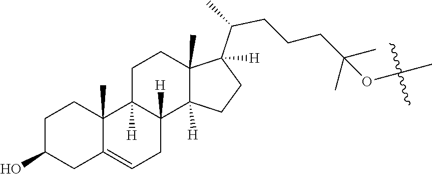 |
| 514 | 2 | 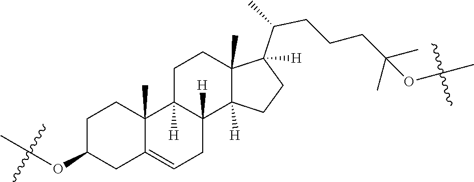 |
| 515 | 1 | 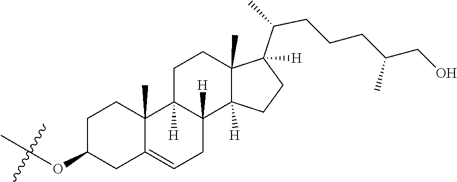 |
| 516 | 1 | 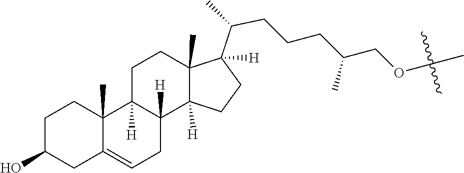 |
| 517 | 2 | 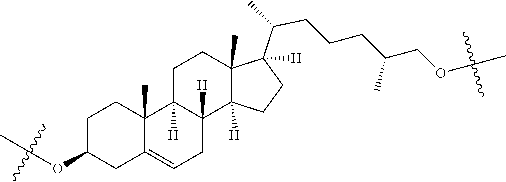 |
| 518 | 1 | 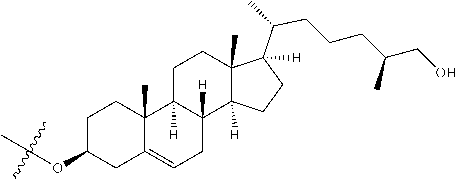 |
| 519 | 1 | 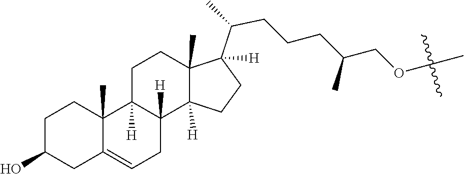 |
| 520 | 2 | 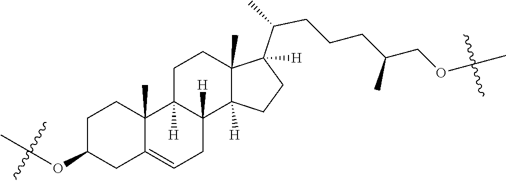 |
| 521 | 1 | 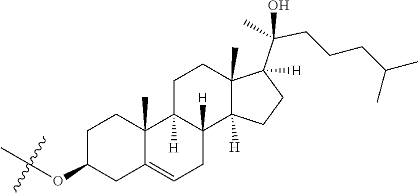 |
| 522 | 1 | 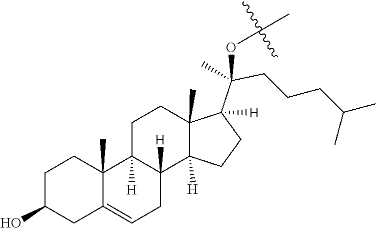 |
| 523 | 2 | 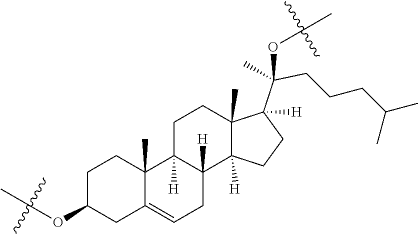 |
| 524 | 1 | 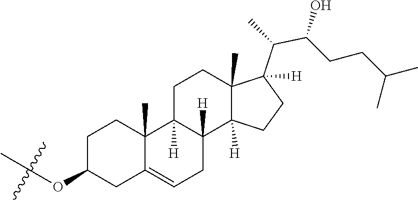 |
| 525 | 1 | 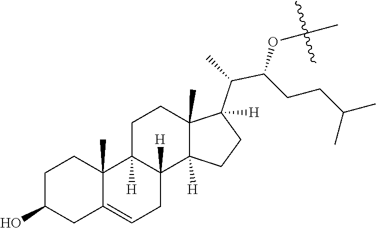 |
| 526 | 2 | 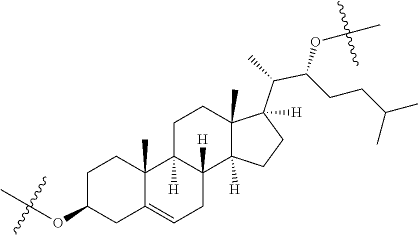 |
| 527 | 1 | 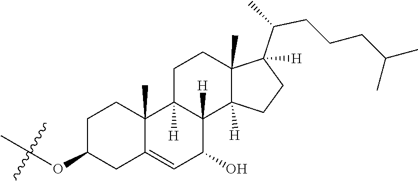 |
| 528 | 1 | 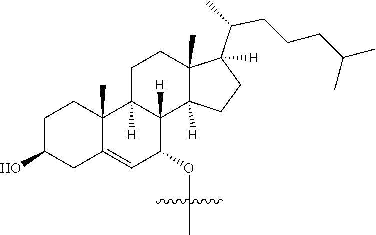 |
| 529 | 2 | 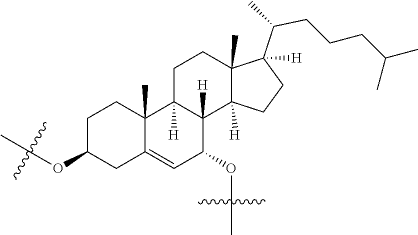 |
| 530 | 1 | 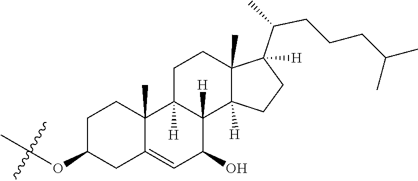 |
| 531 | 1 | 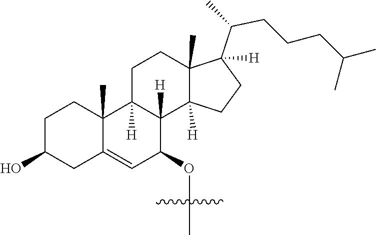 |
| 532 | 2 | 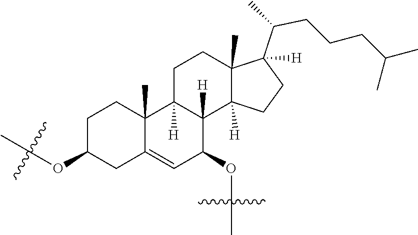 |
| 533 | 1 | 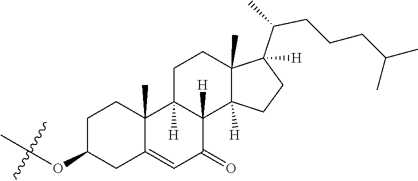 |
| 534 | 1 | 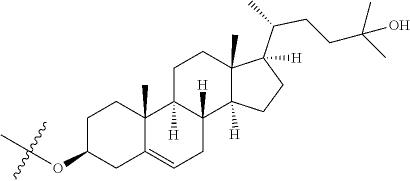 |
| 535 | 1 | 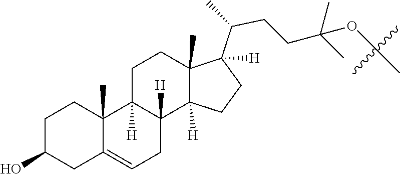 |
| 536 | 2 | 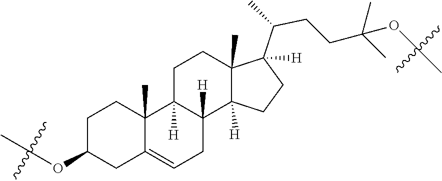 |
| 537 | 1 | 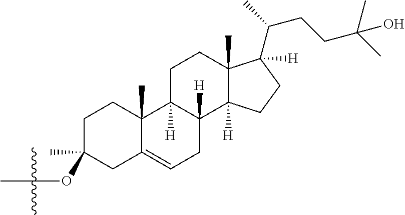 |
| 538 | 1 | 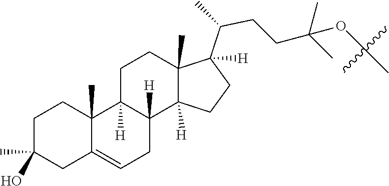 |
| 539 | 2 | 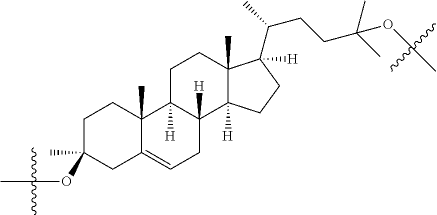 |
| 540 | 1 | 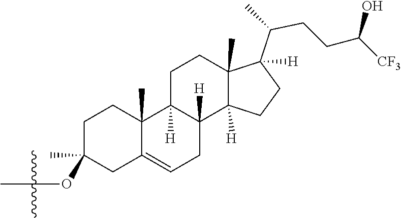 |
| 541 | 1 | 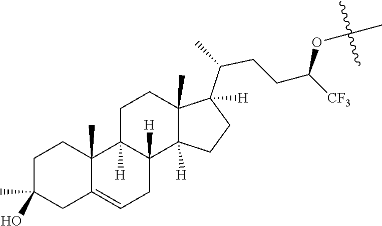 |
| 542 | 2 | 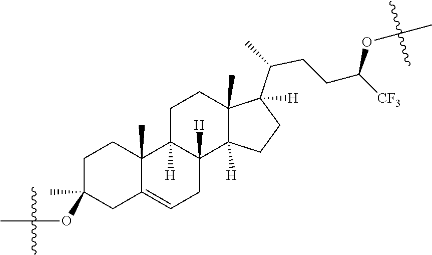 |
| 543 | 1 | 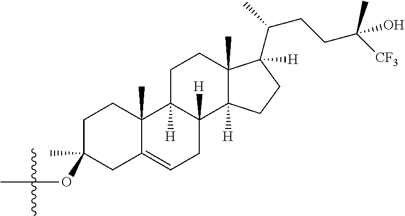 |
| 544 | 1 | 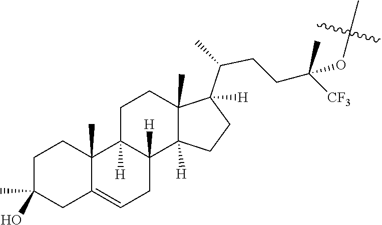 |
| 545 | 2 | 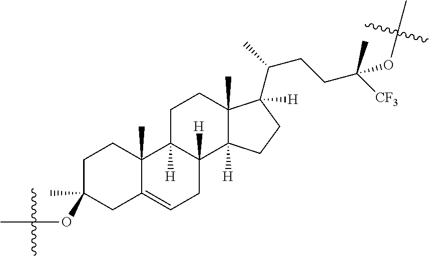 |
| 546 | 1 | 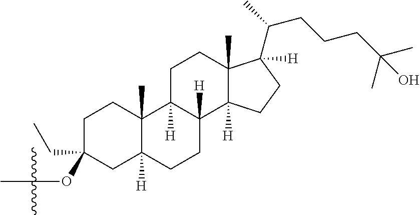 |
| 547 | 1 | 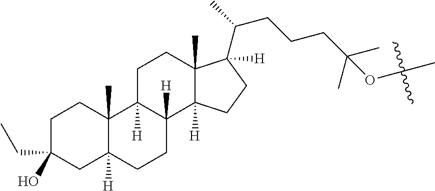 |
| 548 | 2 | 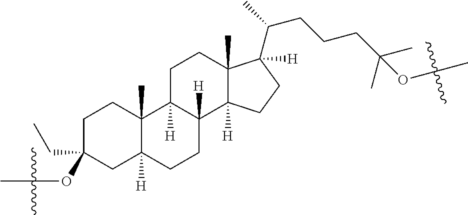 |
| 549 | 1 | 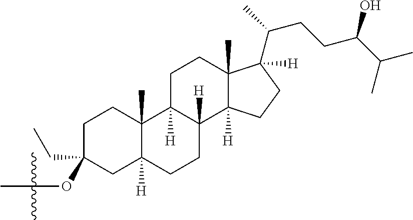 |
| 550 | 1 | 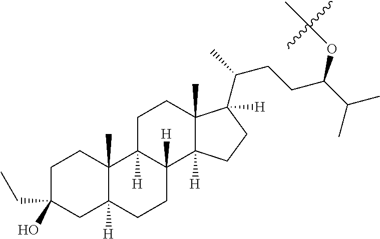 |
| 551 | 2 | 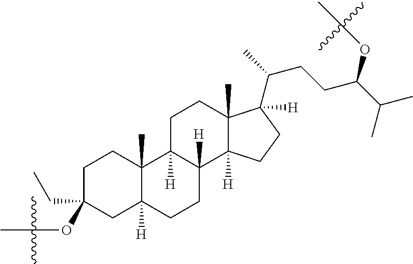 |
| 552 | 1 | 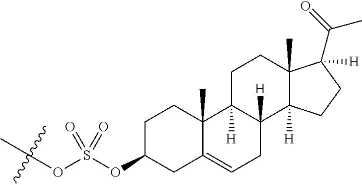 |
| 553 | 1 | 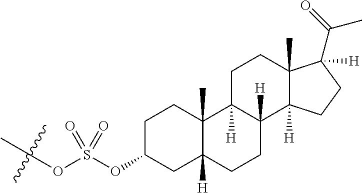 |
| 554 | 1 | 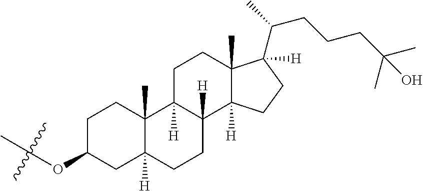 |
| 555 | 1 | 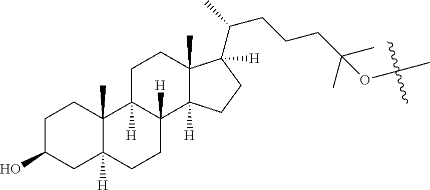 |
| 556 | 2 | 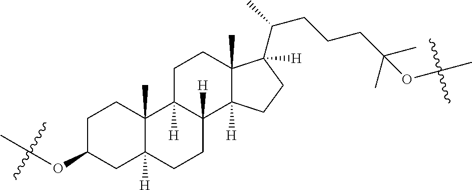 |
| 557 | 1 | 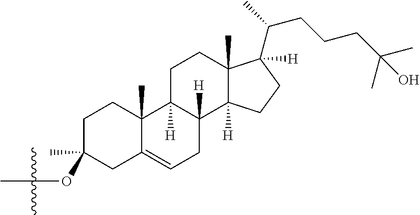 |
| 558 | 1 | 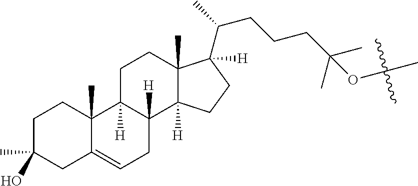 |
| 559 | 2 | 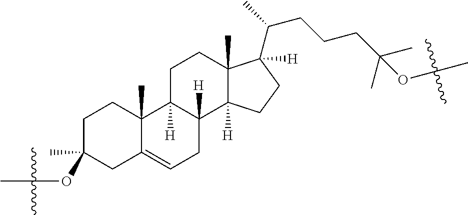 |
| 560 | 1 | 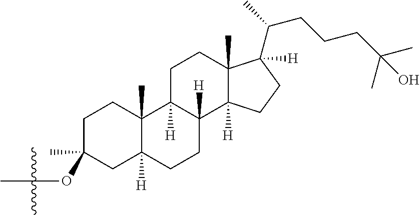 |
| 561 | 1 | 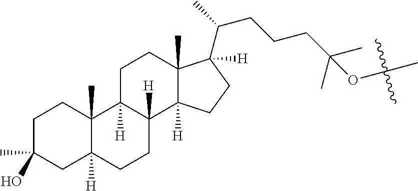 |
| 562 | 2 | 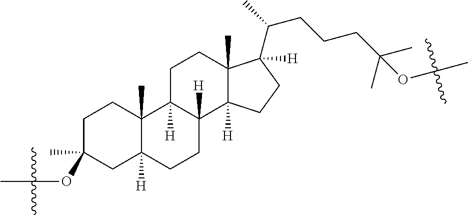 |
| 563 | 1 | 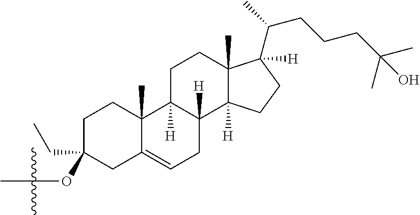 |
| 564 | 1 | 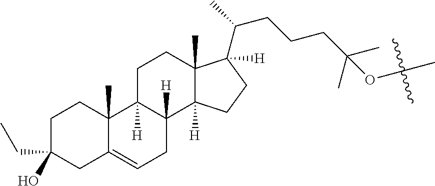 |
| 565 | 2 | 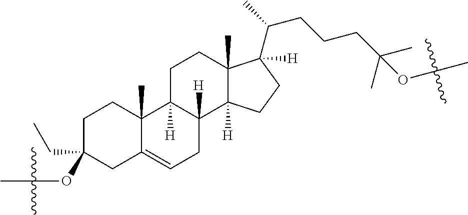 |
| 566 | 1 | 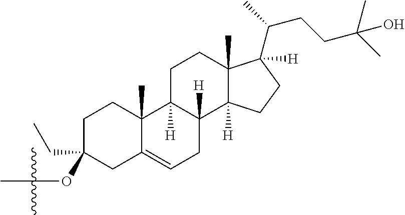 |
| 567 | 1 | 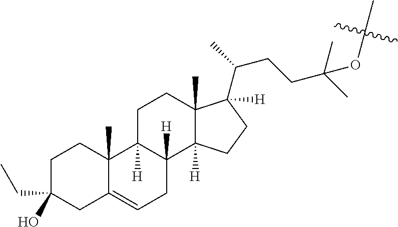 |
| 568 | 2 | 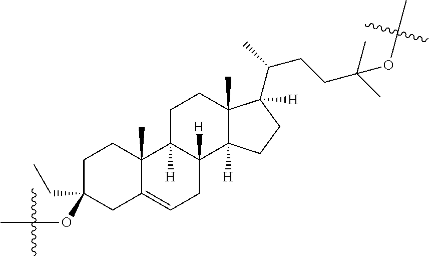 |

In some embodiments, the compound is selected from any of the compounds depicted in in Table 10, pharmaceutically acceptable salts thereof, deuterated variants thereof or combinations thereof.

TABLE 10

| | |
|---|---|
| | Formula (XXXVI) |
| 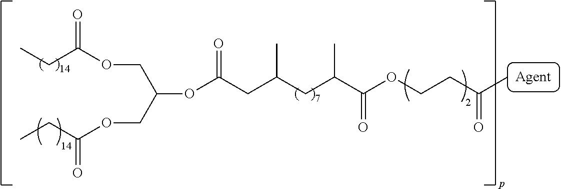 | |

| Cpd. No. | p | 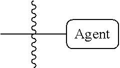 |
|---|---|---|
| 569 | 1 | 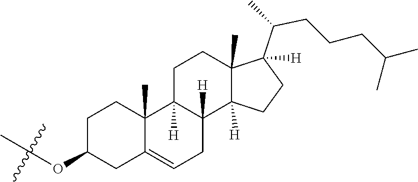 |
| 570 | 1 | 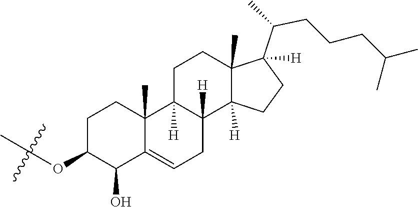 |
| 571 | 1 | 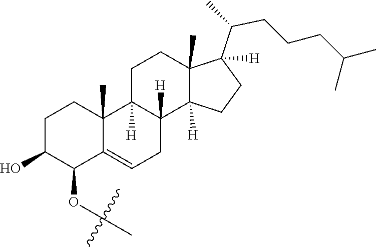 |
| 572 | 2 | 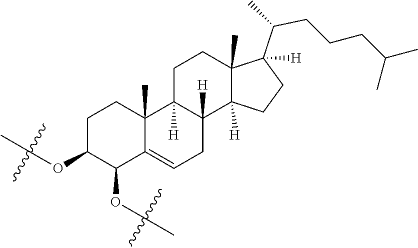 |
| 573 | 1 | 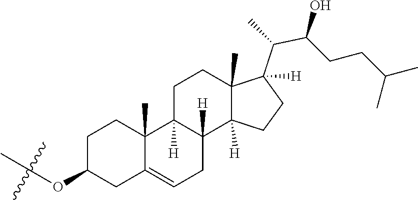 |
| 574 | 1 | 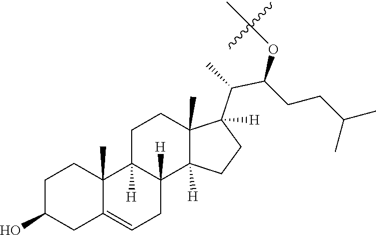 |
| 575 | 2 | 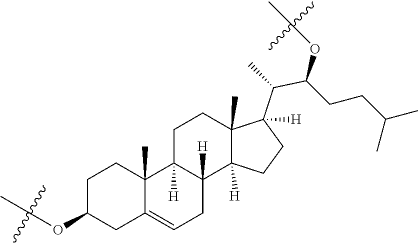 |
| 576 | 1 | 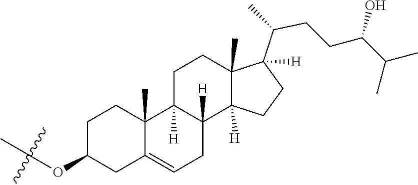 |
| 577 | 1 | 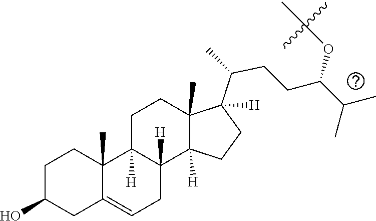 |
| 578 | 2 | 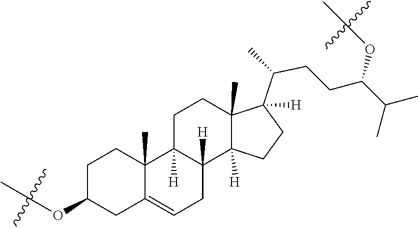 |
| 579 | 1 | 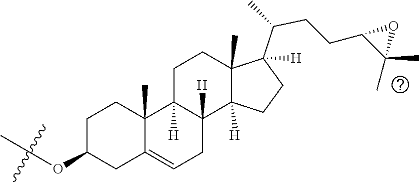 |
| 580 | 1 | 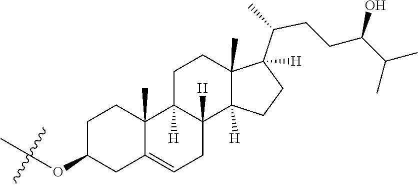 |
| 581 | 1 | 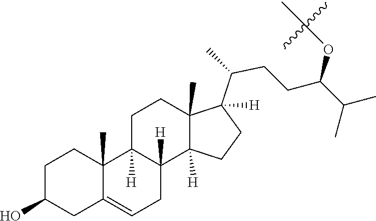 |
| 582 | 2 | 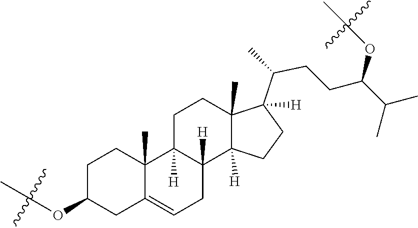 |
| 583 | 1 | 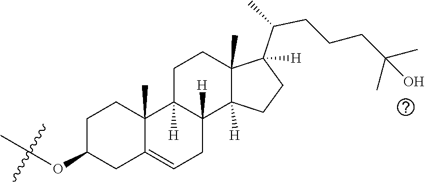 |
| 584 | 1 | 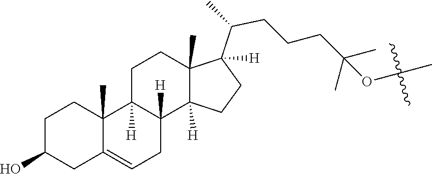 |
| 585 | 2 | 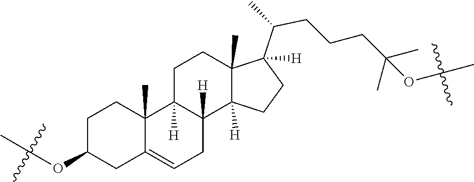 |
| 586 | 1 | 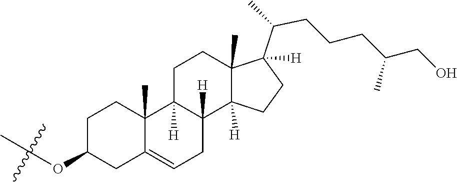 |
| 587 | 1 | 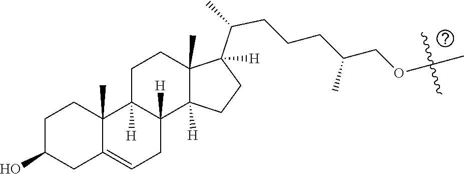 |
| 588 | 2 | 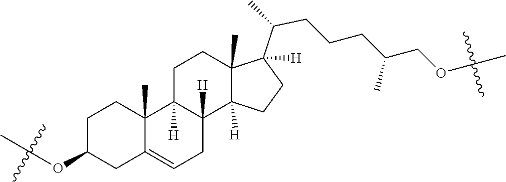 |
| 589 | 1 | 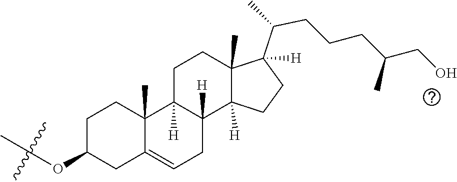 |
| 590 | 1 | 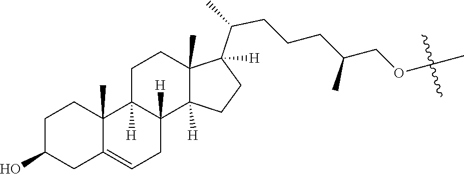 |
| 591 | 2 | 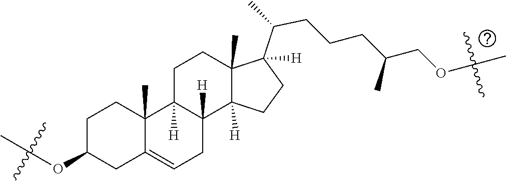 |
| 592 | 1 | 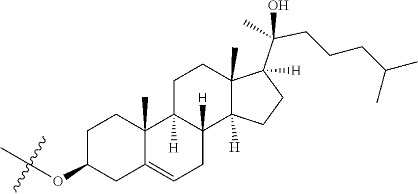 |
| 593 | 1 | 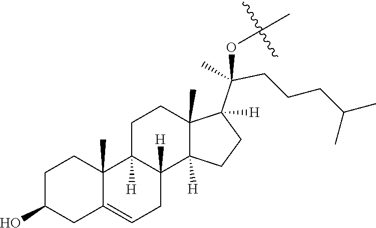 |
| 594 | 2 | 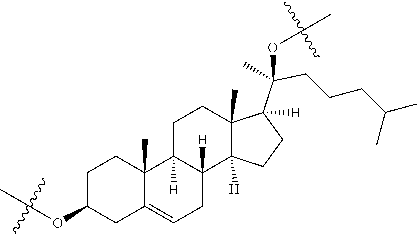 |
| 595 | 1 | 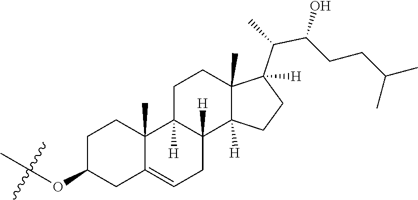 |
| 596 | 1 | 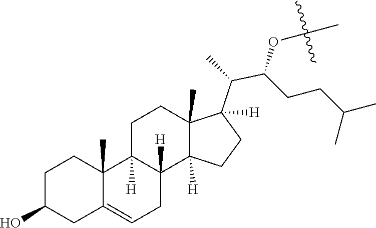 |
| 597 | 2 | 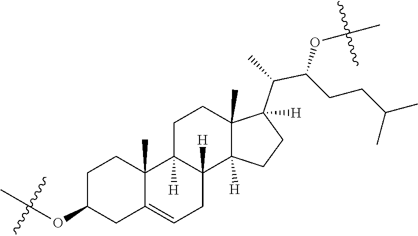 |
| 598 | 1 | 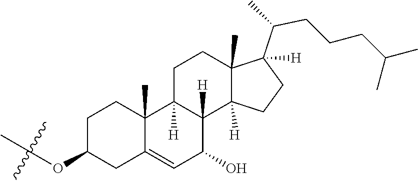 |
| 599 | 1 | 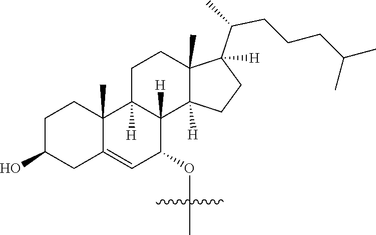 |
| 600 | 2 | 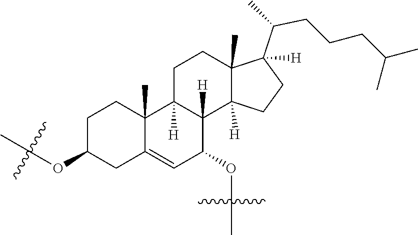 |
| 601 | 1 | 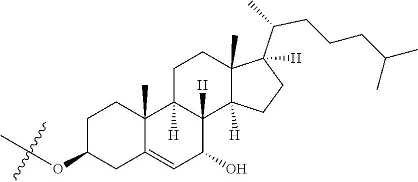 |
| 602 | 1 | 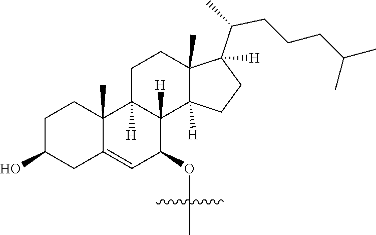 |
| 603 | 2 | 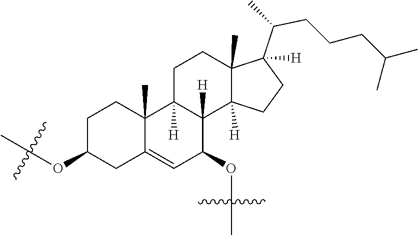 |
| 604 | 1 | 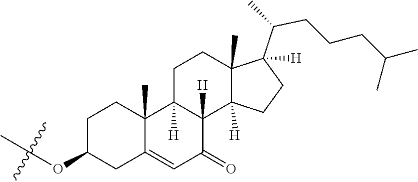 |
| 605 | 1 | 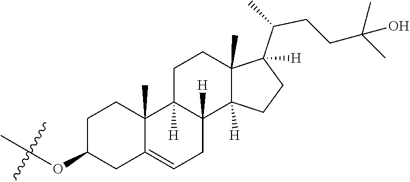 |
| 606 | 1 | 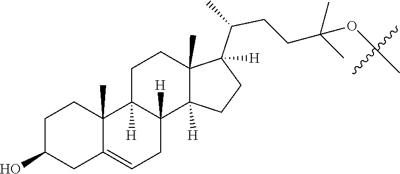 |
| 607 | 2 | 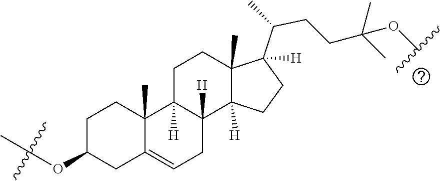 |
| 608 | 1 | 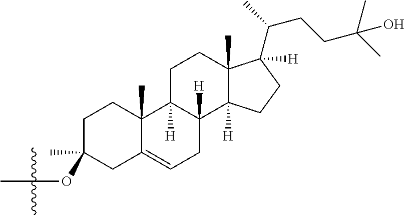 |
| 609 | 1 | 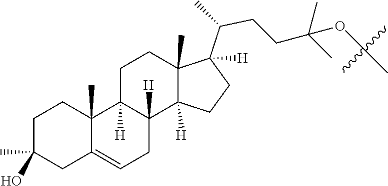 |
| 610 | 2 | 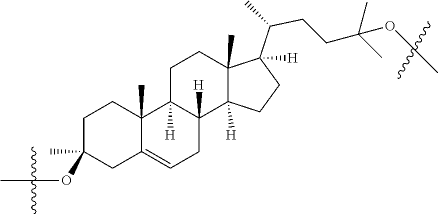 |
| 611 | 1 | 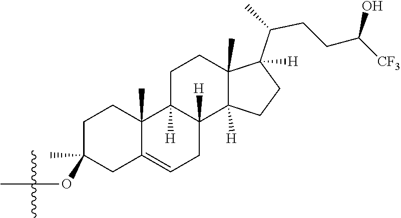 |
| 612 | 1 | 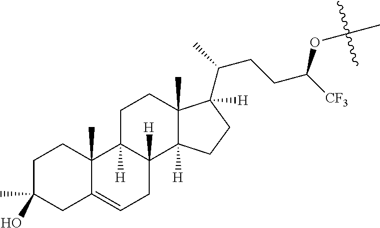 |
| 613 | 2 | 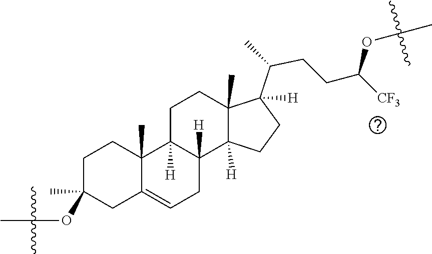 |
| 614 | 1 | 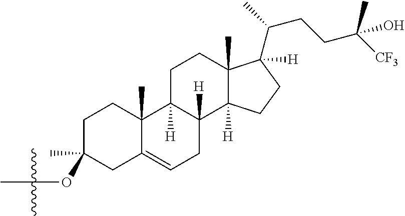 |
| 615 | 1 | 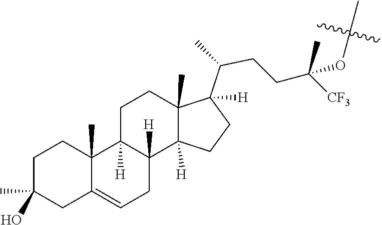 |
| 616 | 2 | 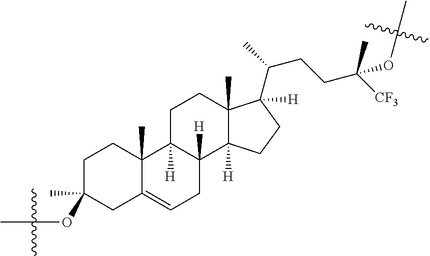 |
| 617 | 1 | 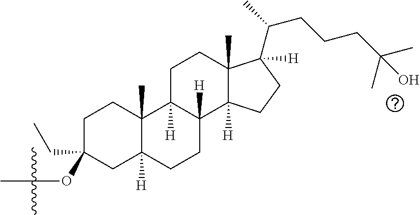 |
| 618 | 1 | 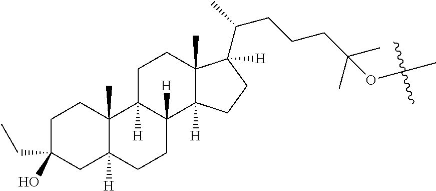 |
| 619 | 2 | 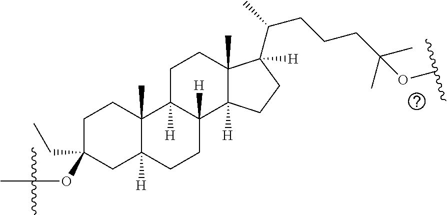 |
| 620 | 1 | 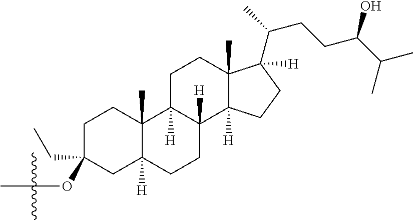 |
| 621 | 1 | 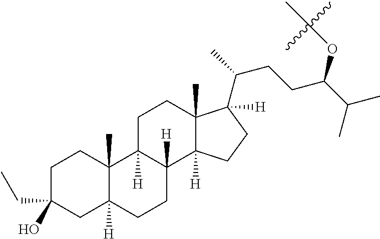 |
| 622 | 2 | 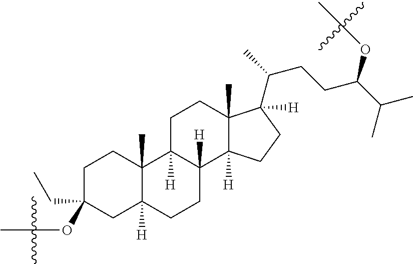 |
| 623 | 1 | 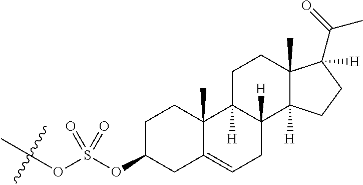 |
| 624 | 1 | 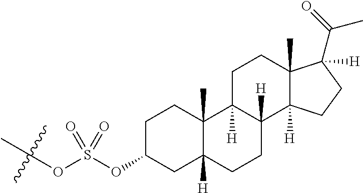 |
| 625 | 1 | 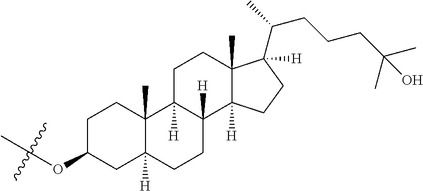 |
| 626 | 1 | 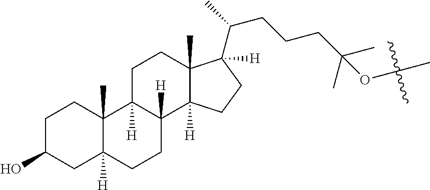 |
| 627 | 2 | 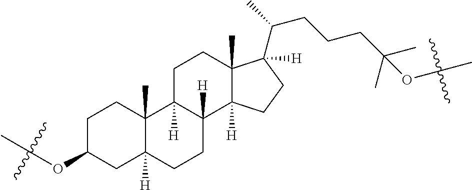 |
| 628 | 1 | 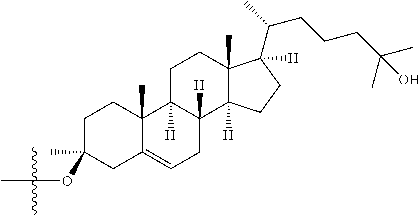 |
| 629 | 1 | 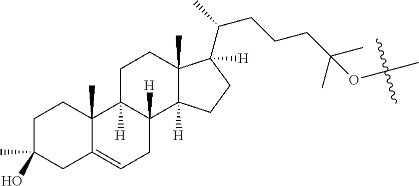 |
| 630 | 2 | 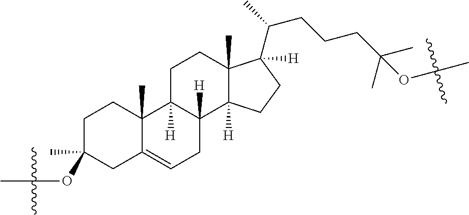 |
| 631 | 1 | 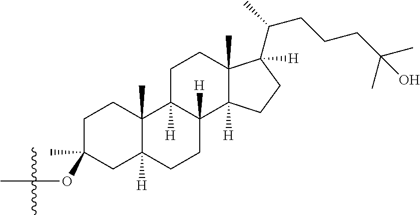 |
| 632 | 1 | 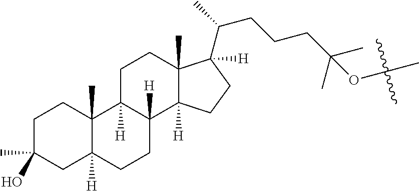 |
| 633 | 2 | 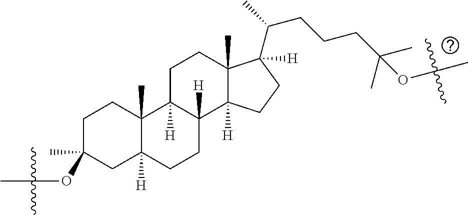 |
| 634 | 1 | 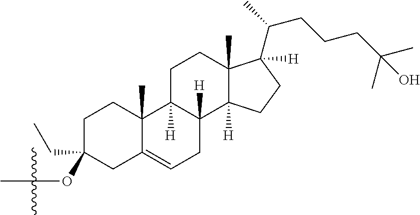 |
| 635 | 1 | 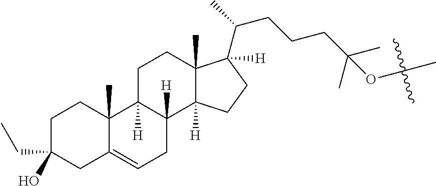 |
| 636 | 2 | 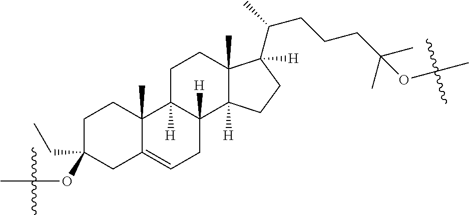 |
| 637 | 1 | 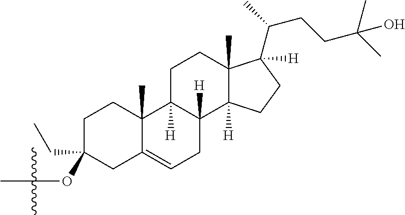 |
| 638 | 1 | 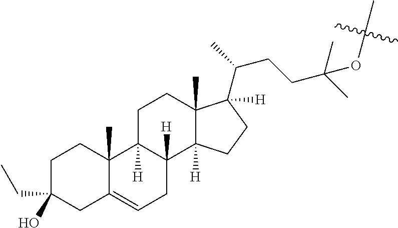 |
| 639 | 2 | 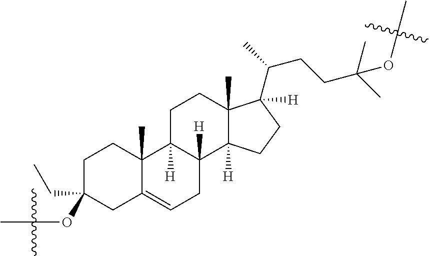 |

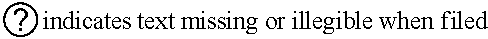

In some embodiments, the compound is selected from any of the compounds depicted in in Table 11, pharmaceutically acceptable salts thereof, deuterated variants thereof or combinations thereof.

TABLE 11

| | |
|---|---|
| | Formula (XXXVII) |
| 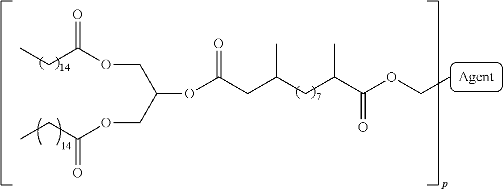 | |

| Cpd. No. | p | 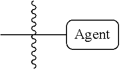 |
|---|---|---|
| 640 | 1 | 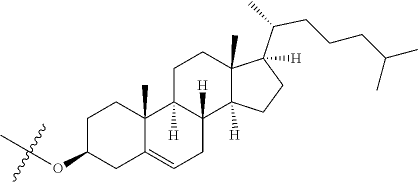 |
| 641 | 1 | 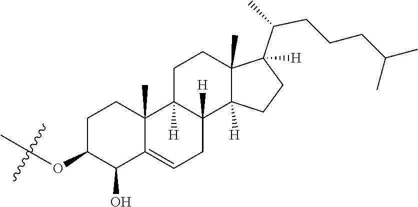 |
| 642 | 1 | 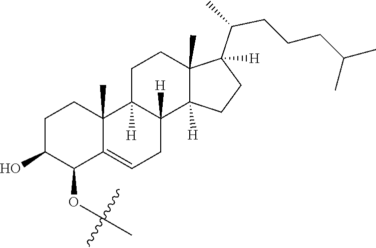 |
| 643 | 2 | 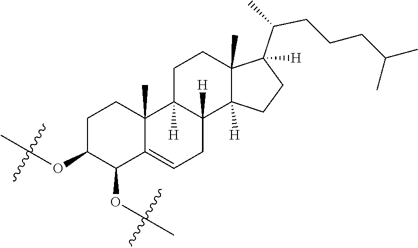 |
| 644 | 1 | 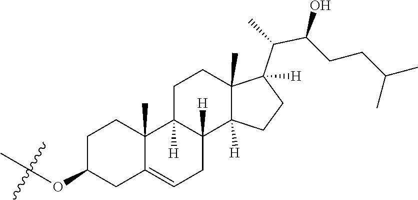 |
| 645 | 1 | 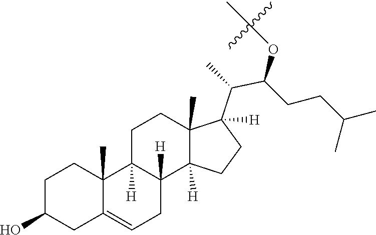 |
| 646 | 2 | 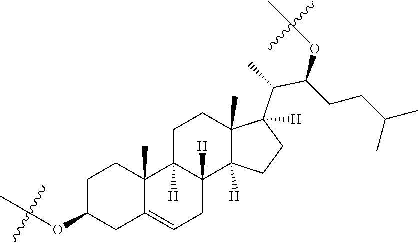 |
| 647 | 1 | 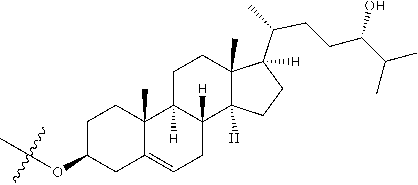 |
| 648 | 1 | 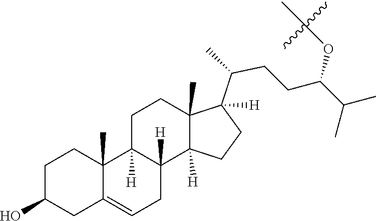 |
| 649 | 2 | 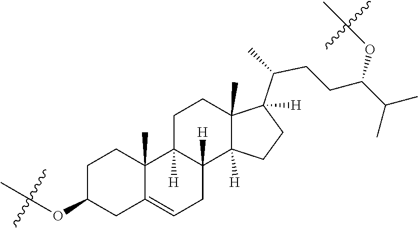 |
| 650 | 1 | 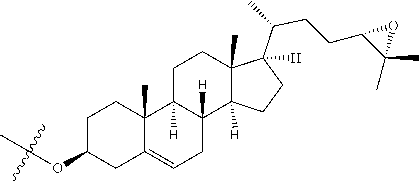 |
| 651 | 1 | 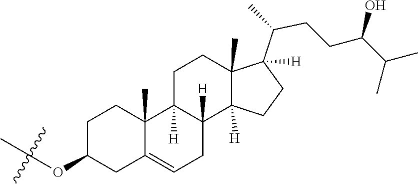 |
| 652 | 1 | 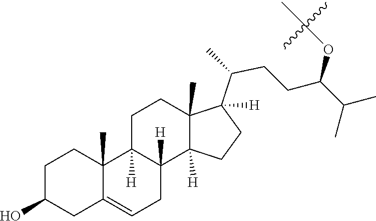 |
| 653 | 2 | 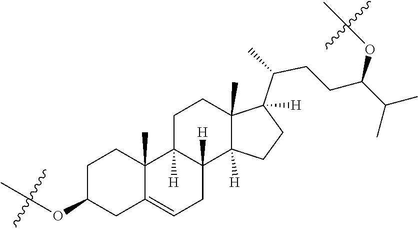 |
| 654 | 1 | 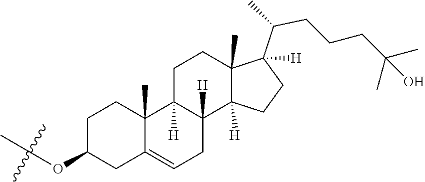 |
| 655 | 1 | 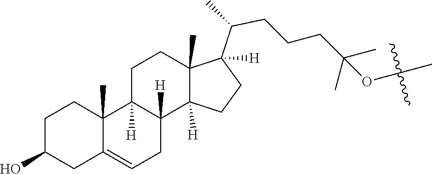 |
| 656 | 2 | 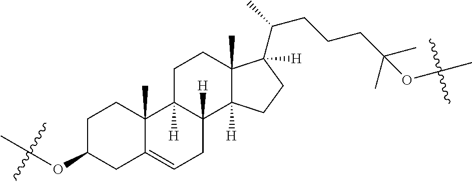 |
| 657 | 1 | 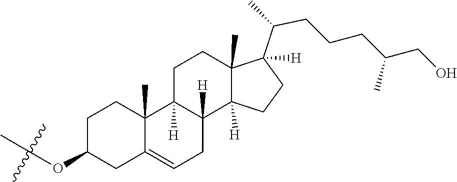 |
| 658 | 1 | 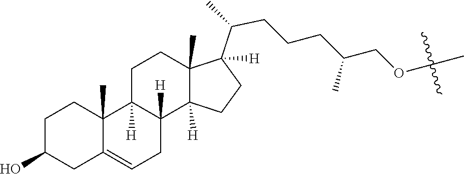 |
| 659 | 2 | 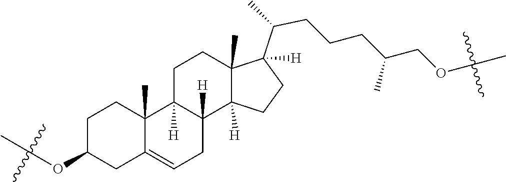 |
| 660 | 1 | 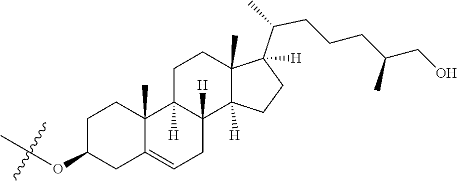 |
| 661 | 1 | 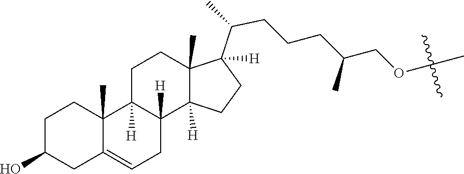 |
| 662 | 2 | 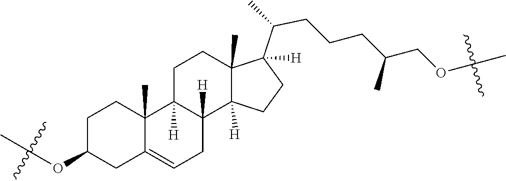 |
| 663 | 1 | 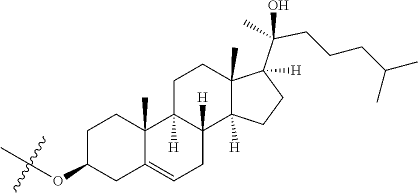 |
| 664 | 1 | 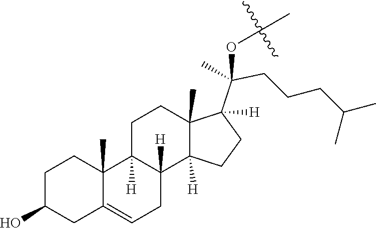 |
| 665 | 2 | 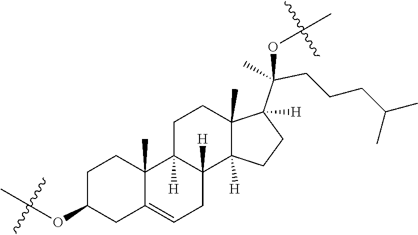 |
| 666 | 1 | 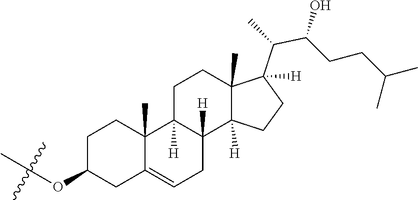 |
| 667 | 1 | 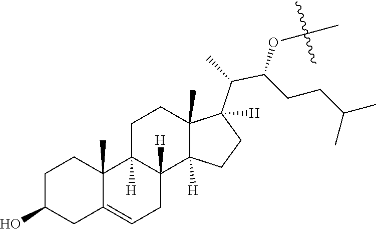 |
| 668 | 2 | 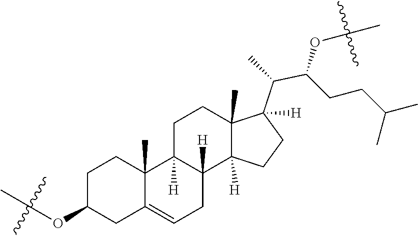 |
| 669 | 1 | 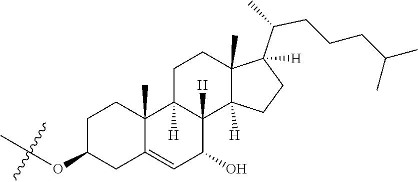 |
| 670 | 1 | 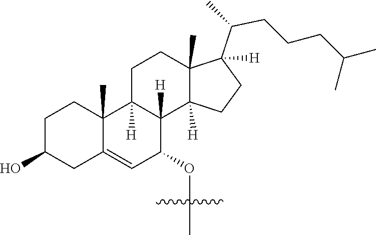 |
| 671 | 2 | 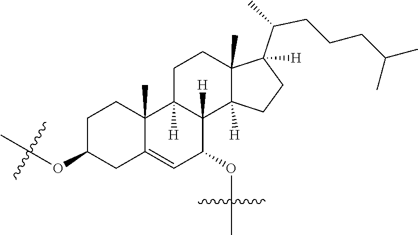 |
| 672 | 1 | 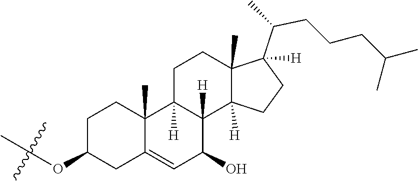 |
| 673 | 1 | 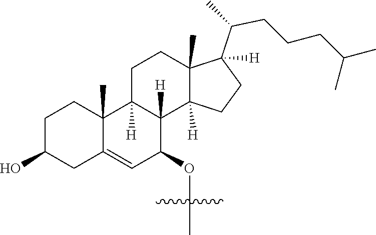 |
| 674 | 2 | 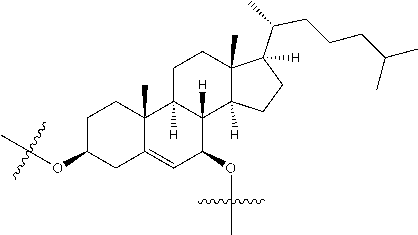 |
| 675 | 1 | 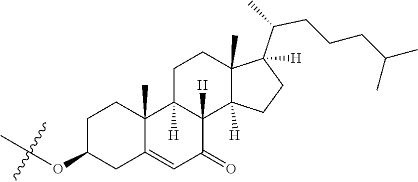 |
| 676 | 1 | 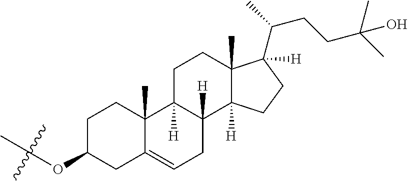 |
| 677 | 1 | 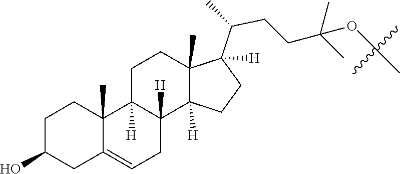 |
| 678 | 2 | 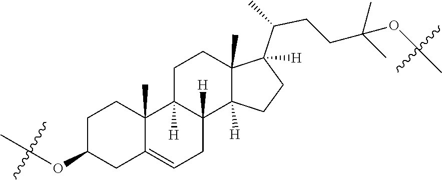 |
| 679 | 1 | 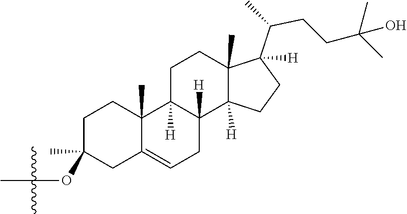 |
| 680 | 1 | 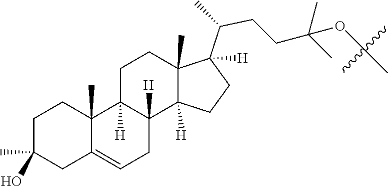 |
| 681 | 2 | 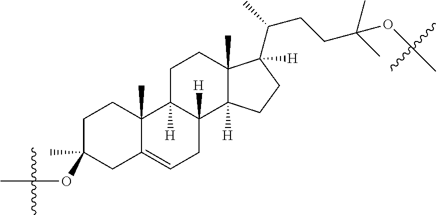 |
| 682 | 1 | 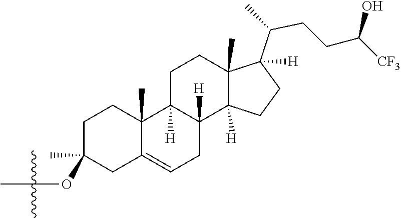 |
| 683 | 1 | 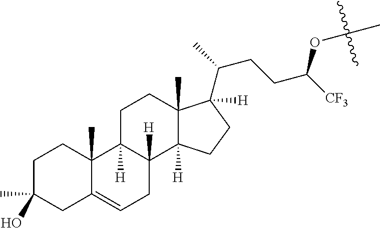 |
| 684 | 2 | 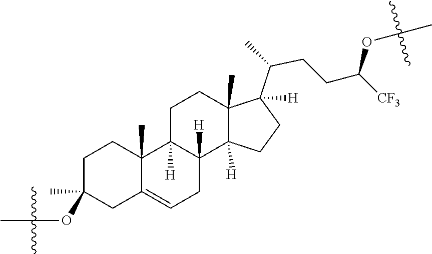 |
| 685 | 1 | 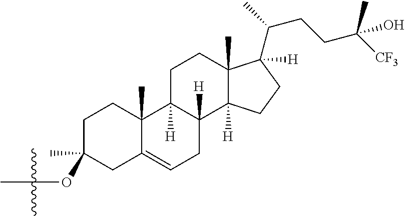 |
| 686 | 1 | 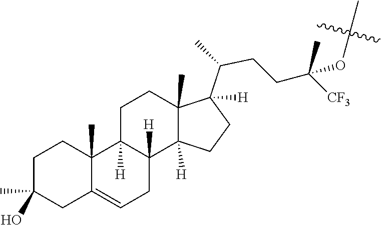 |
| 687 | 2 | 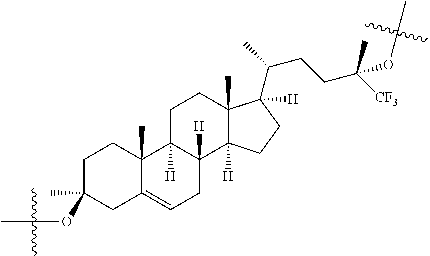 |
| 688 | 1 | 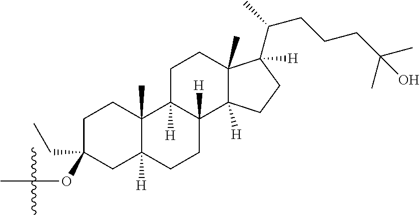 |
| 689 | 1 | 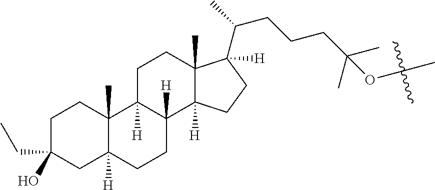 |
| 690 | 2 | 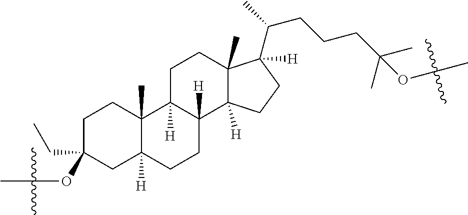 |
| 691 | 1 | 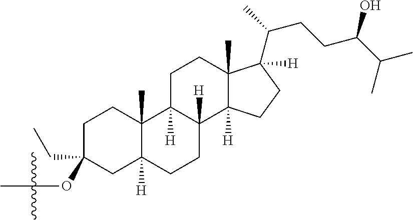 |
| 692 | 1 | 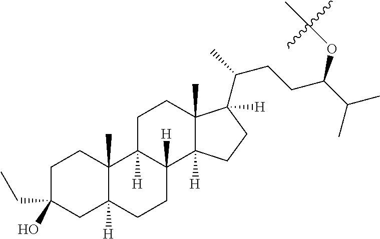 |
| 693 | 2 | 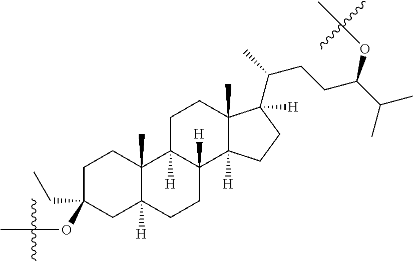 |
| 694 | 1 | 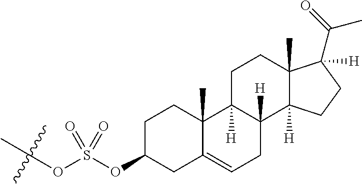 |
| 695 | 1 | 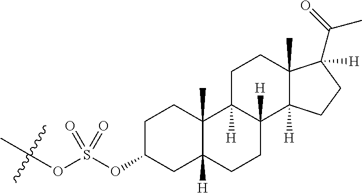 |
| 696 | 1 | 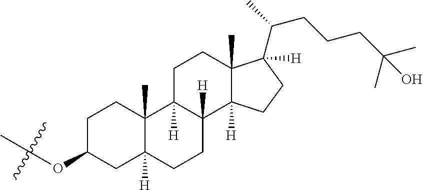 |
| 697 | 1 | 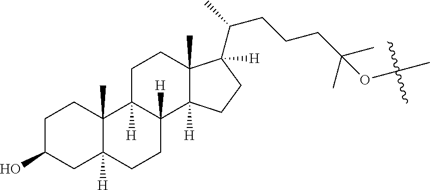 |
| 698 | 2 | 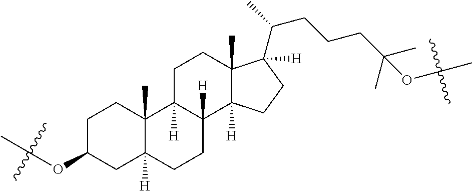 |
| 699 | 1 | 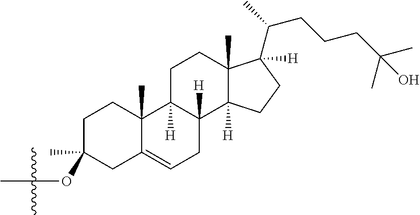 |
| 700 | 1 | 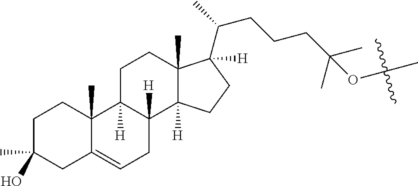 |
| 701 | 2 | 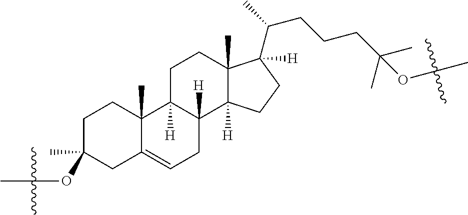 |
| 702 | 1 | 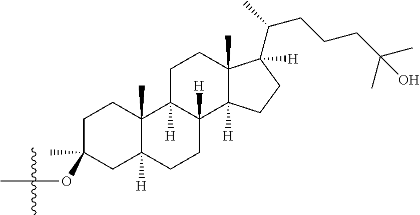 |
| 703 | 1 | 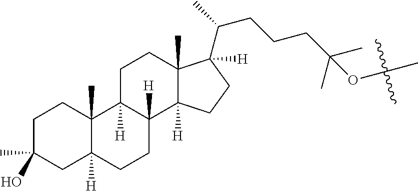 |
| 704 | 2 | 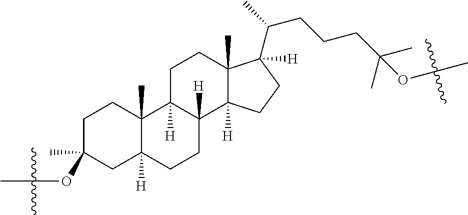 |
| 705 | 1 | 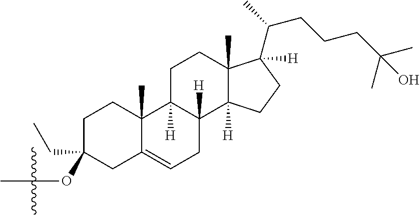 |
| 706 | 1 | 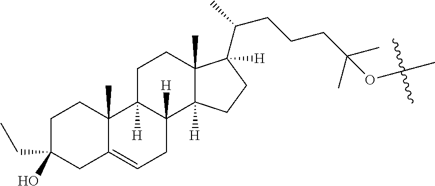 |
| 707 | 2 | 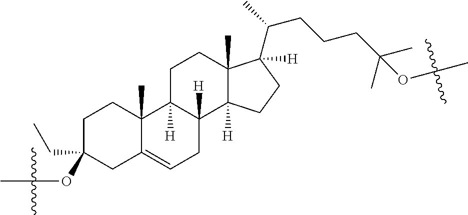 |
| 708 | 1 | 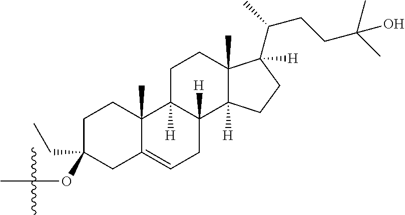 |
| 709 | 1 | 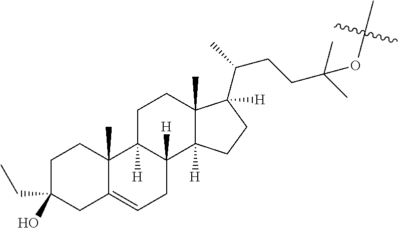 |
| 710 | 2 | 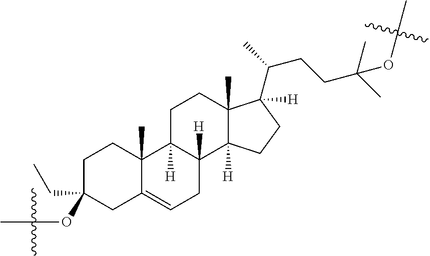 |

In some embodiments, the compound is selected from any of the compounds depicted in in Table 12, pharmaceutically acceptable salts thereof, deuterated variants thereof or combinations thereof.

TABLE 12

| | |
|---|---|
| | Formula (XXXVIII) |
| 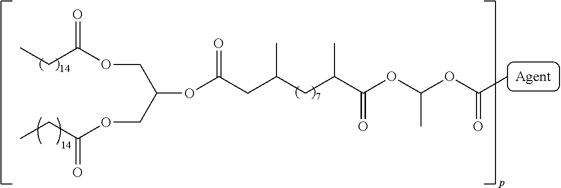 | |

| Cpd. No. | p | 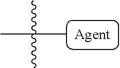 |
|---|---|---|
| 711 | 1 | 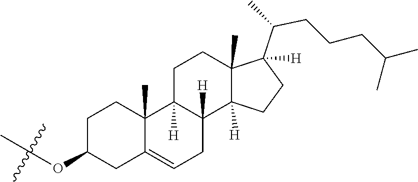 |
| 712 | 1 | 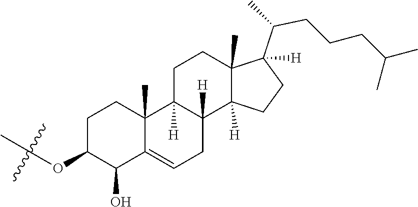 |
| 713 | 1 | 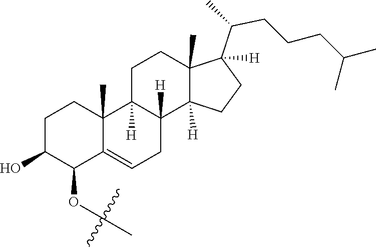 |
| 714 | 2 | 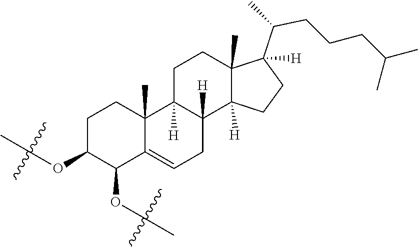 |
| 715 | 1 | 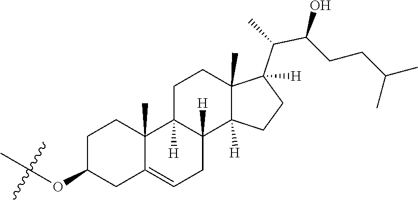 |
| 716 | 1 | 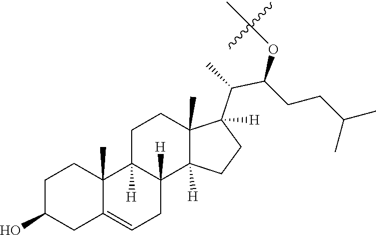 |
| 717 | 2 | 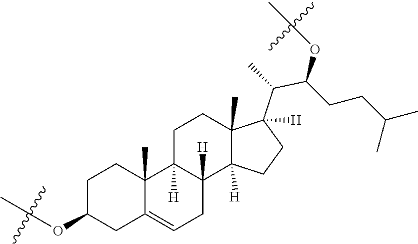 |
| 718 | 1 | 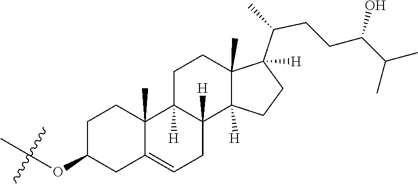 |
| 719 | 1 | 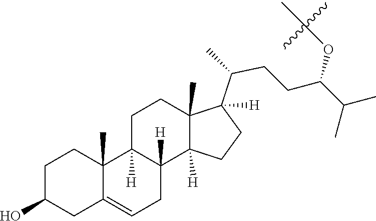 |
| 720 | 2 | 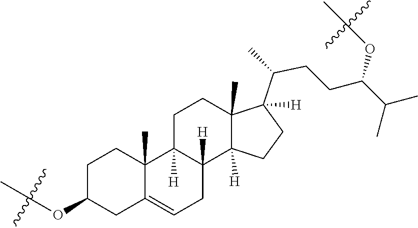 |
| 721 | 1 | 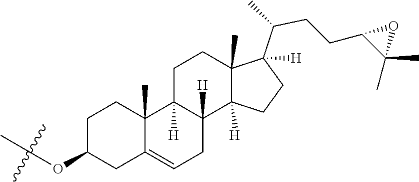 |
| 722 | 1 | 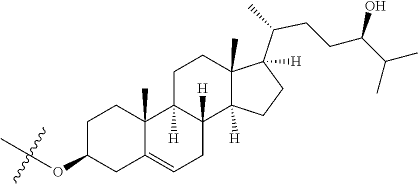 |
| 723 | 1 | 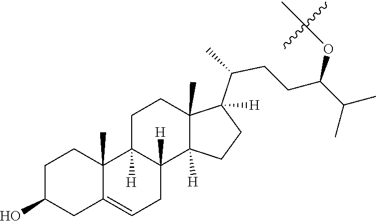 |
| 724 | 2 | 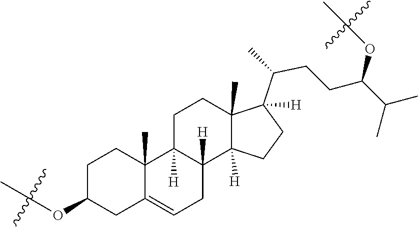 |
| 725 | 1 | 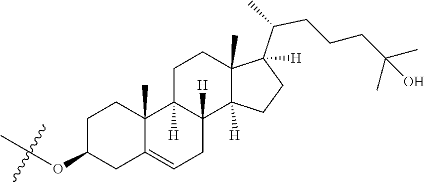 |
| 726 | 1 | 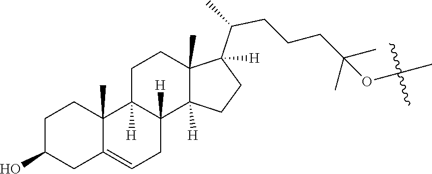 |
| 727 | 2 | 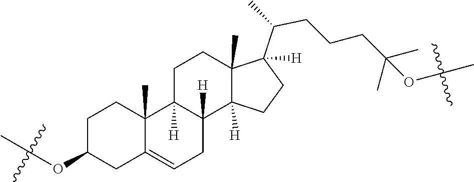 |
| 728 | 1 | 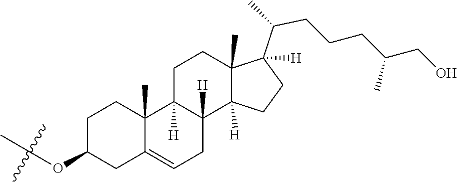 |
| 729 | 1 | 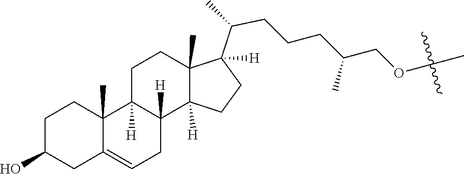 |
| 730 | 2 | 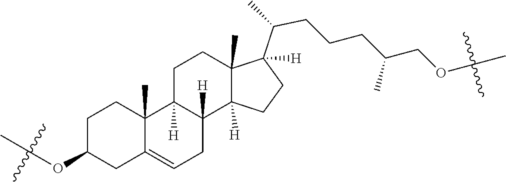 |
| 731 | 1 | 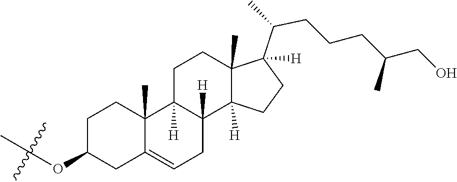 |
| 732 | 1 | 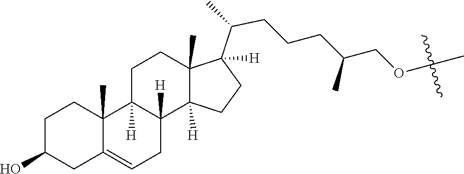 |
| 733 | 2 | 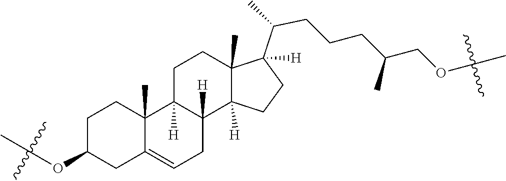 |
| 734 | 1 | 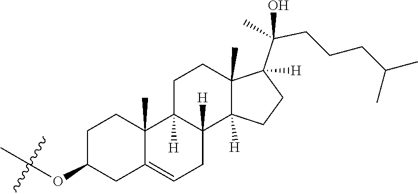 |
| 735 | 1 | 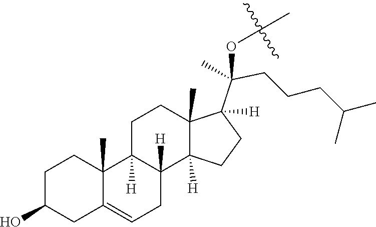 |
| 736 | 2 | 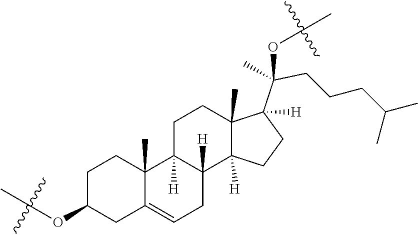 |
| 737 | 1 | 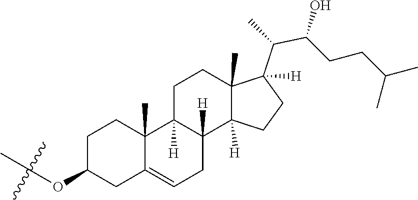 |
| 738 | 1 | 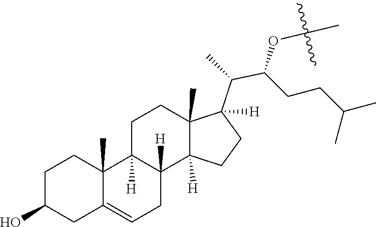 |
| 739 | 2 | 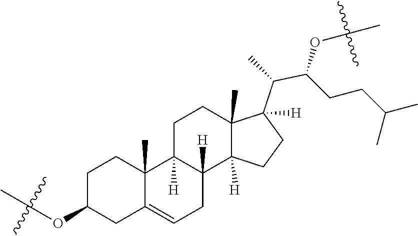 |
| 740 | 1 | 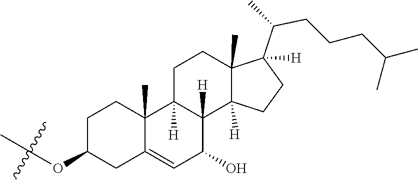 |
| 741 | 1 | 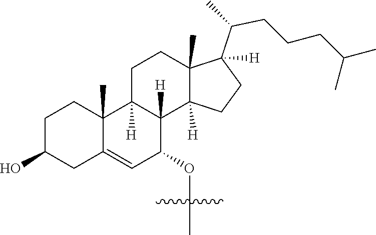 |
| 742 | 2 | 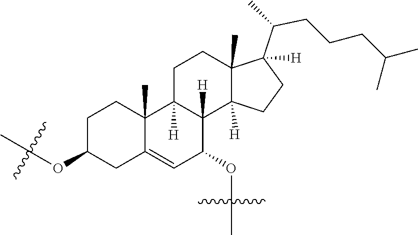 |
| 743 | 1 | 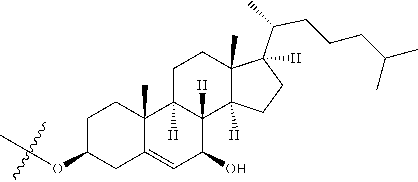 |
| 744 | 1 | 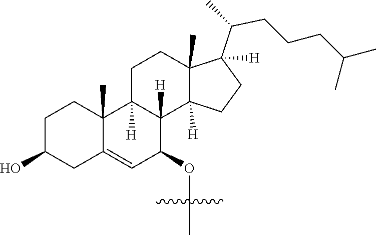 |
| 745 | 2 | 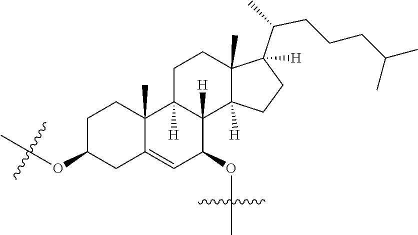 |
| 746 | 1 | 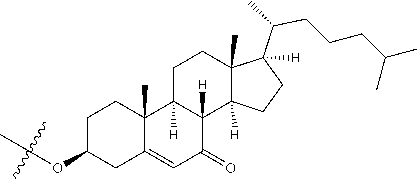 |
| 747 | 1 | 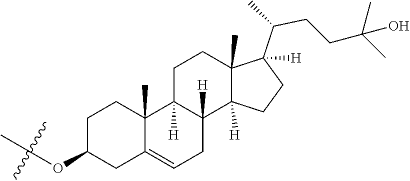 |
| 748 | 1 | 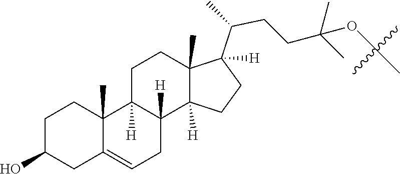 |
| 749 | 2 | 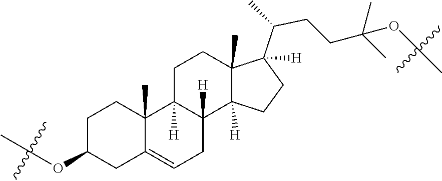 |
| 750 | 1 | 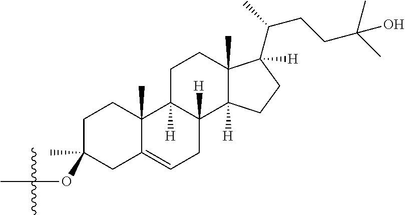 |
| 751 | 1 | 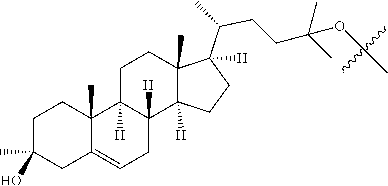 |
| 752 | 2 | 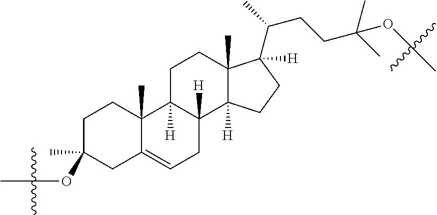 |
| 753 | 1 | 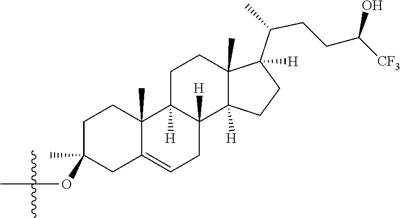 |
| 754 | 1 | 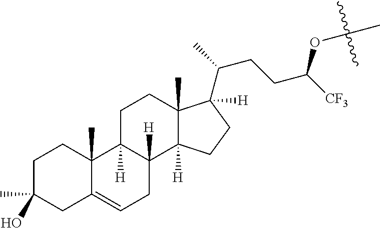 |
| 755 | 2 | 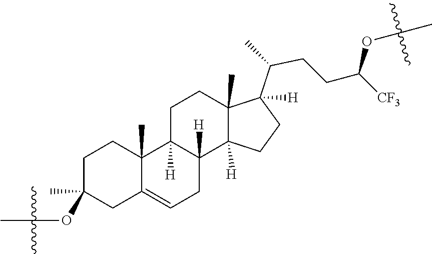 |
| 756 | 1 | 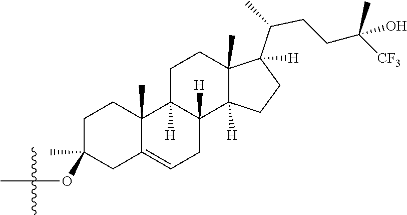 |
| 757 | 1 | 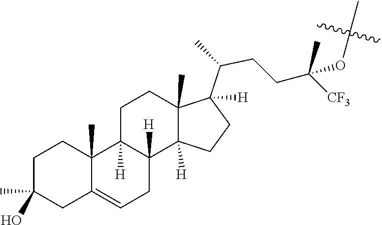 |
| 758 | 2 | 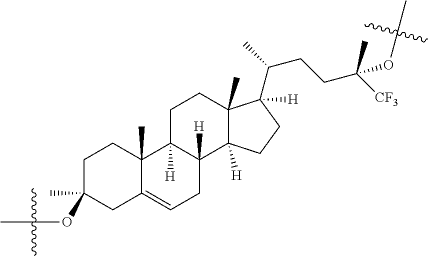 |
| 759 | 1 | 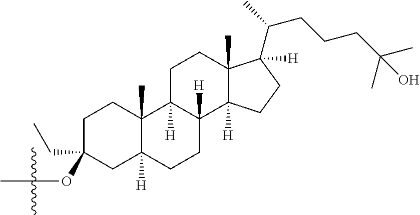 |
| 760 | 1 | 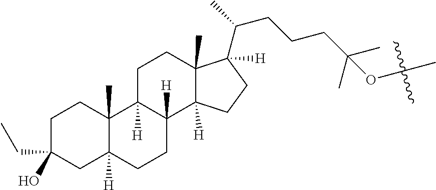 |
| 761 | 2 | 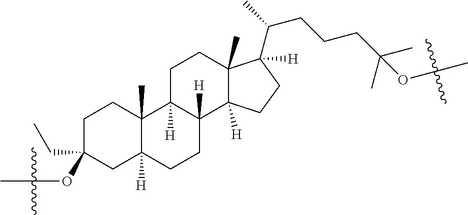 |
| 762 | 1 | 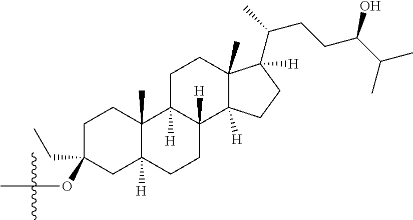 |
| 763 | 1 | 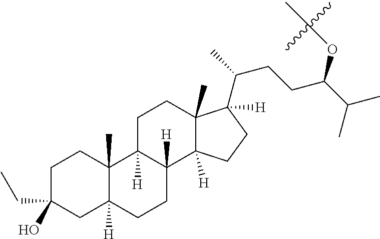 |
| 764 | 2 | 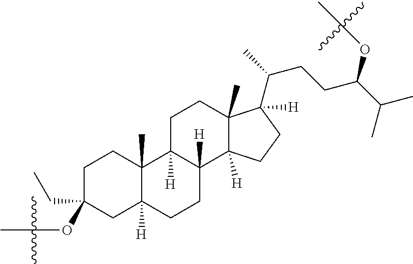 |
| 765 | 1 | 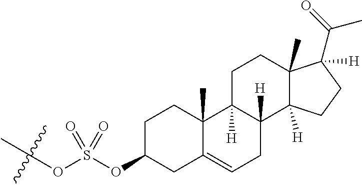 |
| 766 | 1 | 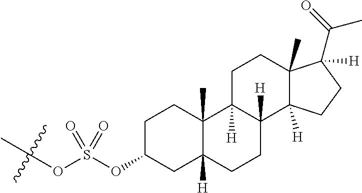 |
| 767 | 1 | 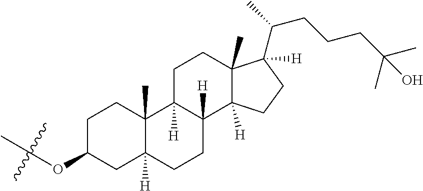 |
| 768 | 1 | 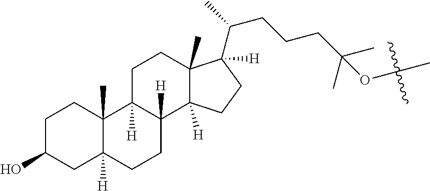 |
| 769 | 2 | 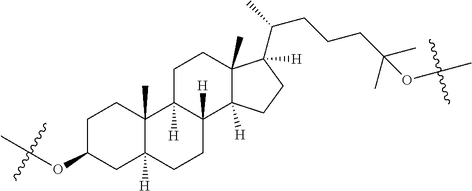 |
| 770 | 1 | 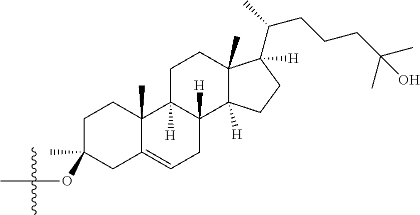 |
| 771 | 1 | 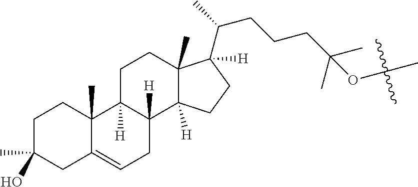 |
| 772 | 2 | 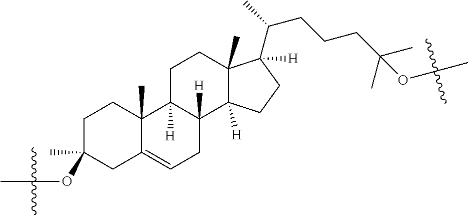 |
| 773 | 1 | 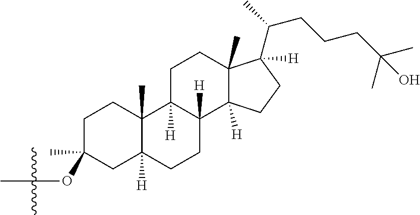 |
| 774 | 1 | 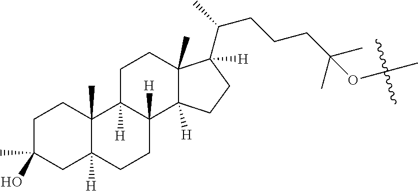 |
| 775 | 2 | 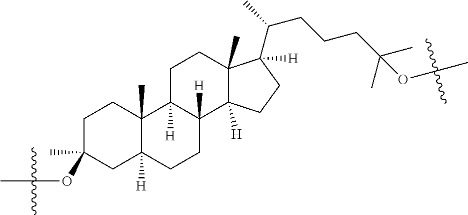 |
| 776 | 1 | 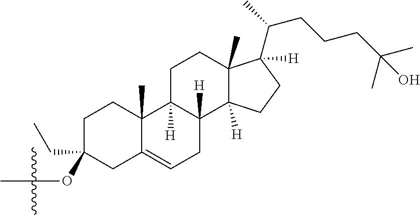 |
| 777 | 1 | 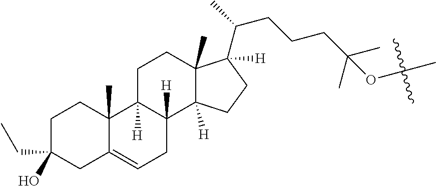 |
| 778 | 2 | 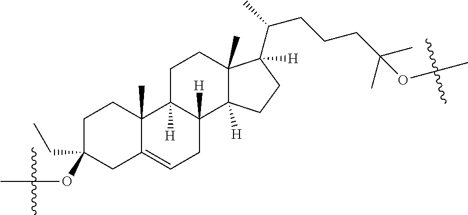 |
| 779 | 1 | 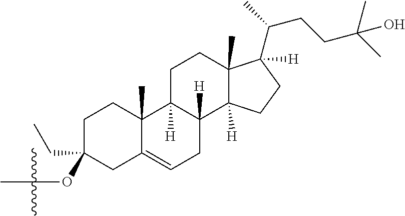 |
| 780 | 1 | 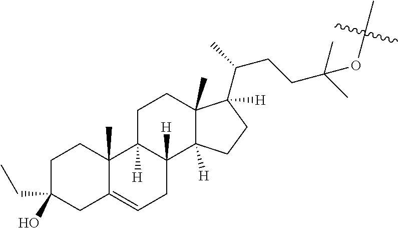 |
| 781 | 2 | 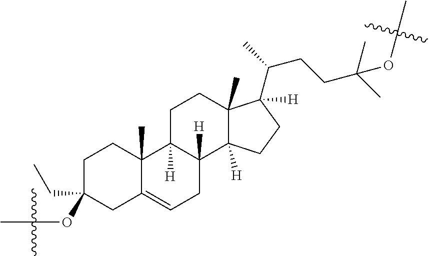 |

In some embodiments, the compound is selected from any of the compounds depicted in in Table 13, pharmaceutically acceptable salts thereof, deuterated variants thereof or combinations thereof.

TABLE 13

| | |
|---|---|
| | Formula (XXXIX) |
| 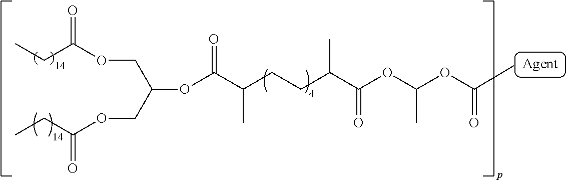 | |

| Cpd. No. | p | 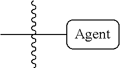 |
|---|---|---|
| 782 | 1 | 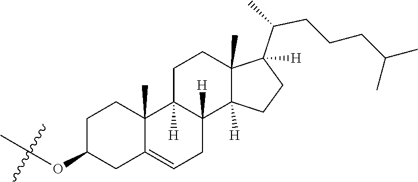 |
| 783 | 1 | 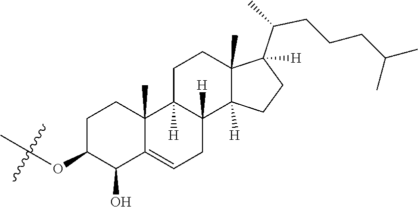 |
| 784 | 1 | 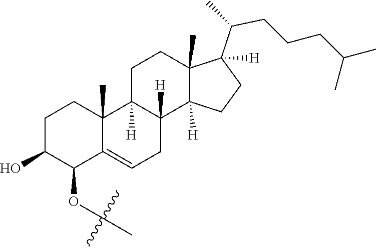 |
| 785 | 2 | 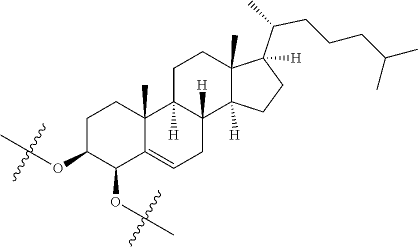 |
| 786 | 1 | 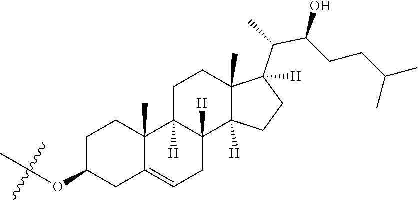 |
| 787 | 1 | 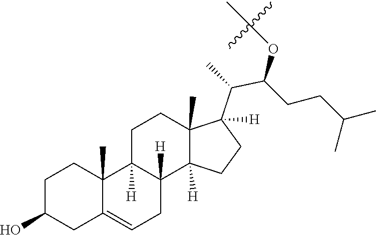 |
| 788 | 2 | 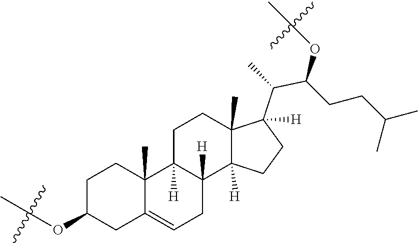 |
| 789 | 1 | 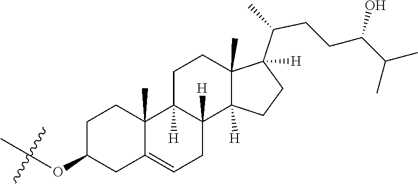 |
| 790 | 1 | 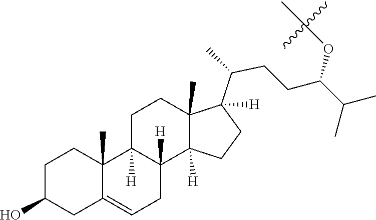 |
| 791 | 2 | 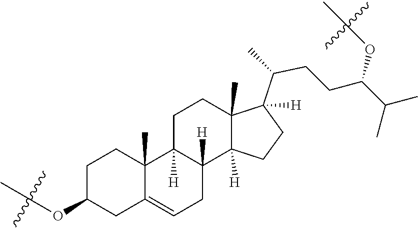 |
| 792 | 1 | 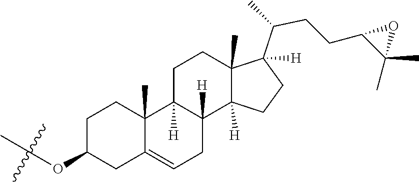 |
| 793 | 1 | 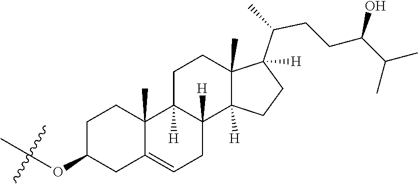 |
| 794 | 1 | 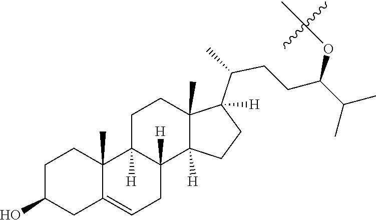 |
| 795 | 2 | 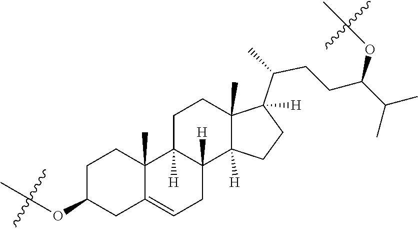 |
| 796 | 1 | 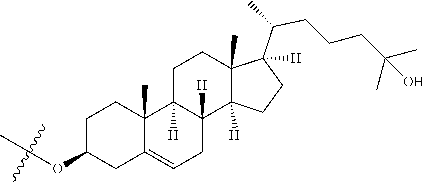 |
| 797 | 1 | 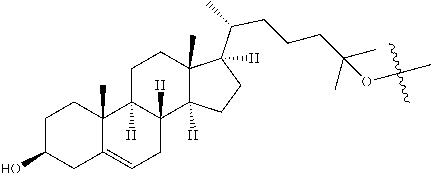 |
| 798 | 2 | 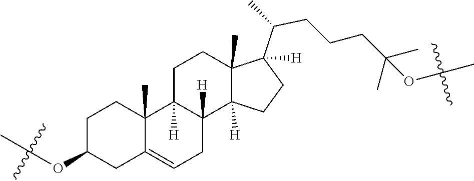 |
| 799 | 1 | 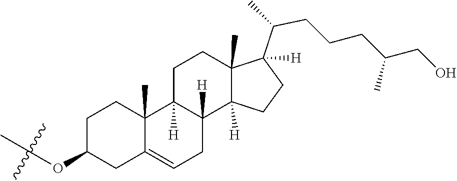 |
| 800 | 1 | 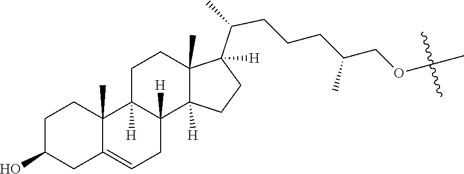 |
| 801 | 2 | 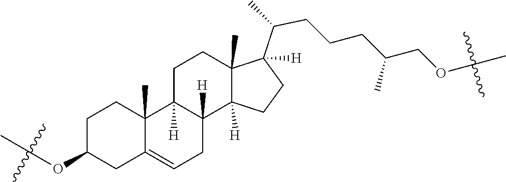 |
| 802 | 1 | 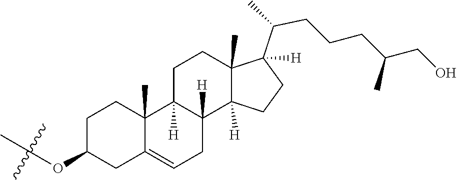 |
| 803 | 1 | 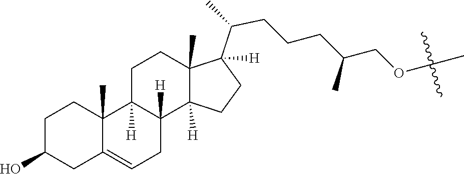 |
| 804 | 2 | 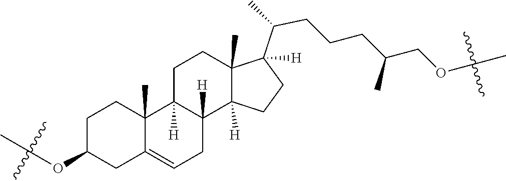 |
| 805 | 1 | 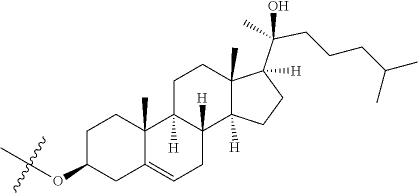 |
| 806 | 1 | 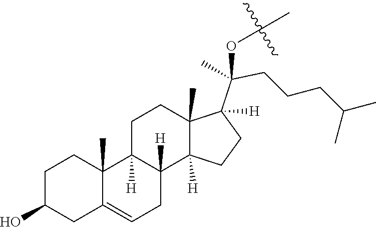 |
| 807 | 2 | 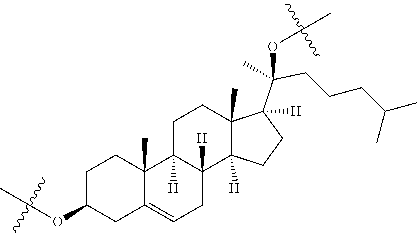 |
| 808 | 1 | 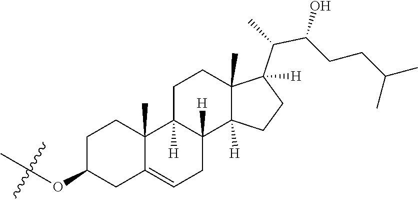 |
| 809 | 1 | 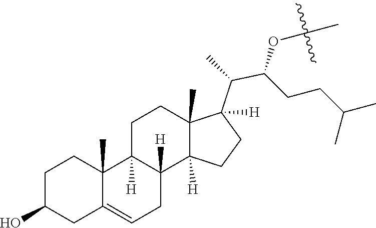 |
| 810 | 2 | 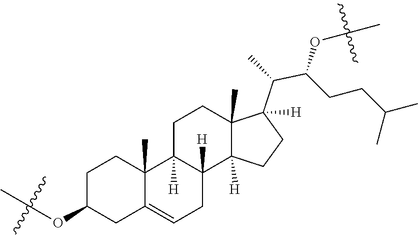 |
| 811 | 1 | 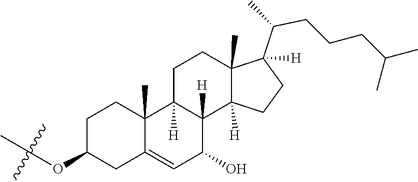 |
| 812 | 1 | 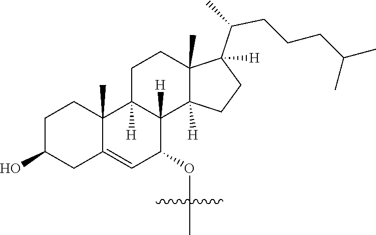 |
| 813 | 2 | 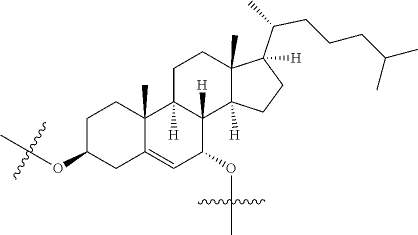 |
| 814 | 1 | 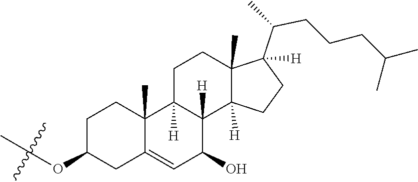 |
| 815 | 1 | 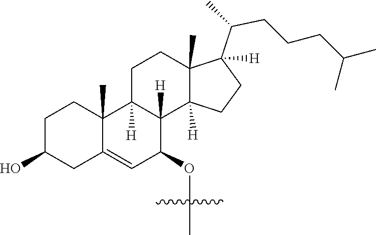 |
| 816 | 2 | 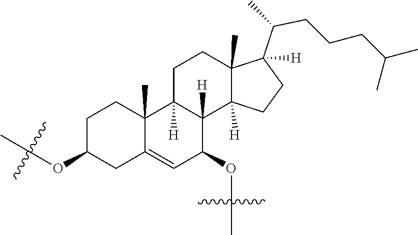 |
| 817 | 1 | 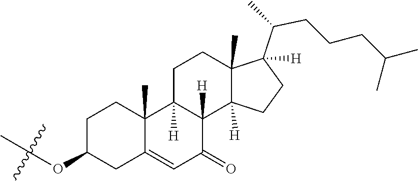 |
| 818 | 1 | 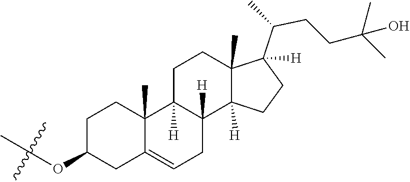 |
| 819 | 1 | 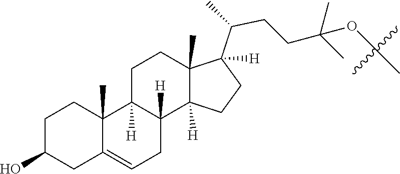 |
| 820 | 2 | 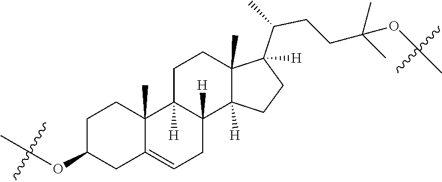 |
| 821 | 1 | 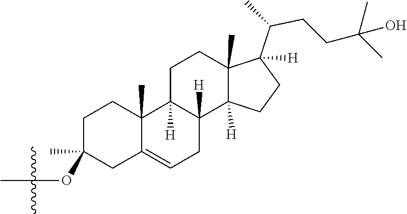 |
| 822 | 1 | 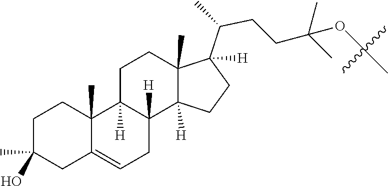 |
| 823 | 2 | 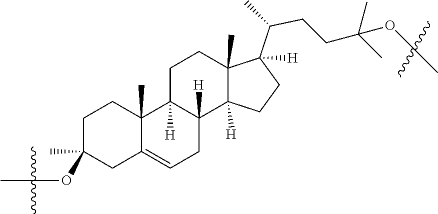 |
| 824 | 1 | 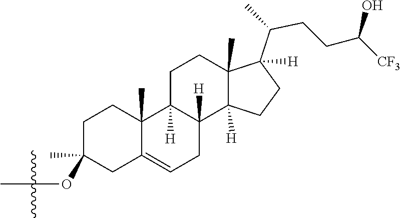 |
| 825 | 1 | 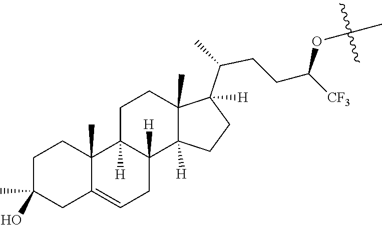 |
| 826 | 2 | 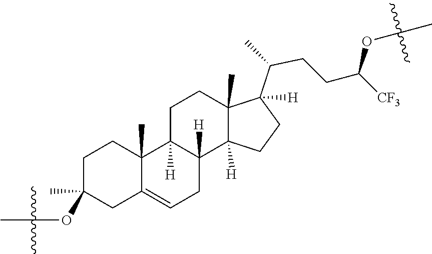 |
| 827 | 1 | 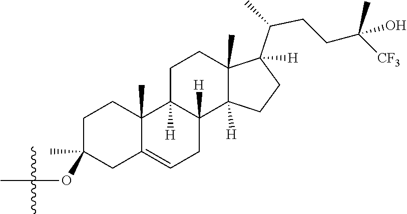 |
| 828 | 1 | 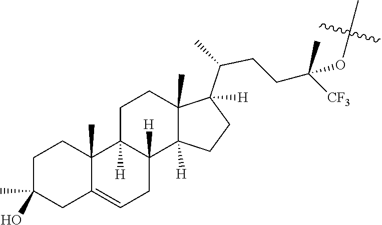 |
| 829 | 2 | 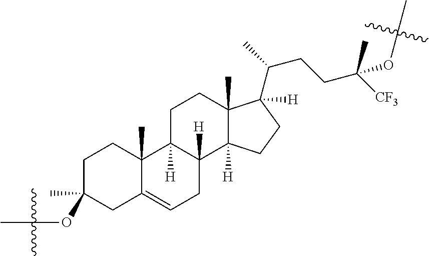 |
| 830 | 1 | 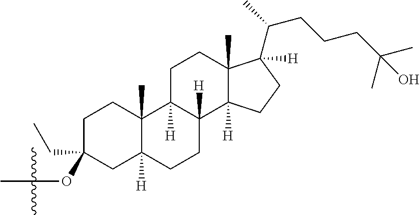 |
| 831 | 1 | 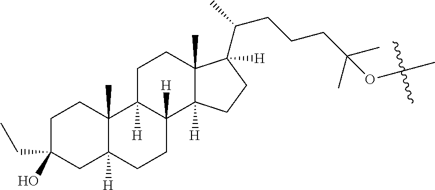 |
| 832 | 2 | 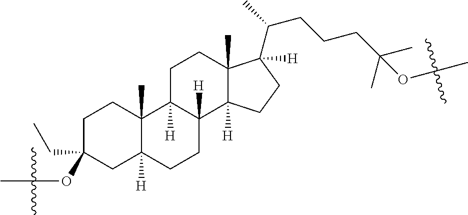 |
| 833 | 1 | 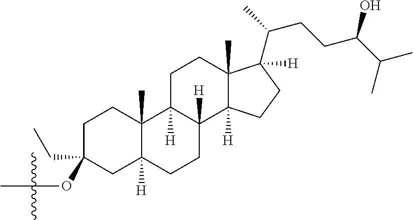 |
| 834 | 1 | 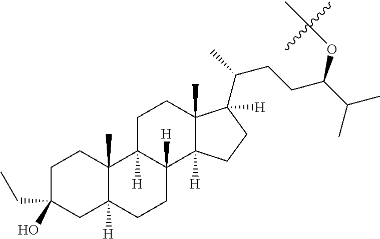 |
| 835 | 2 | 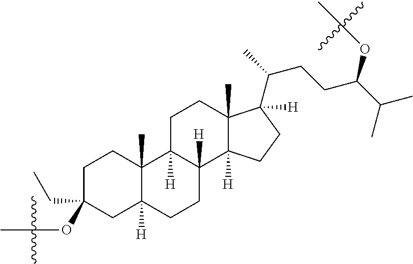 |
| 836 | 1 | 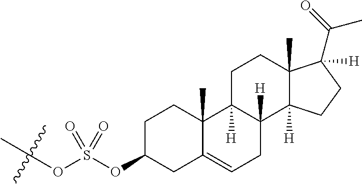 |
| 837 | 1 | 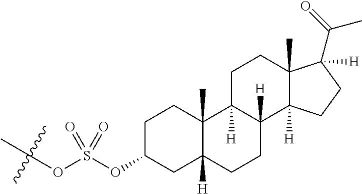 |
| 838 | 1 | 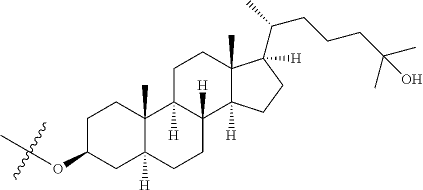 |
| 839 | 1 | 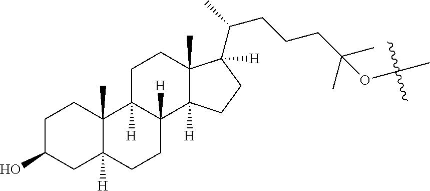 |
| 840 | 2 | 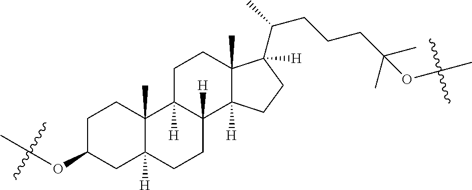 |
| 841 | 1 | 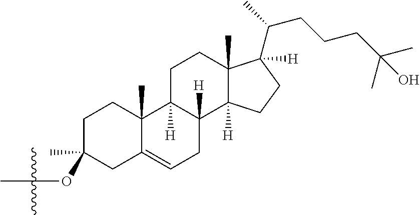 |
| 842 | 1 | 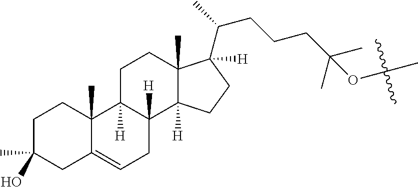 |
| 843 | 2 | 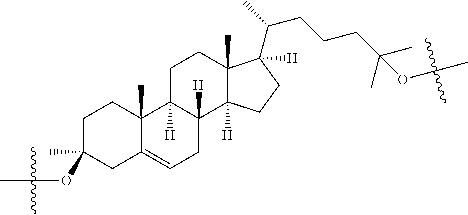 |
| 844 | 1 | 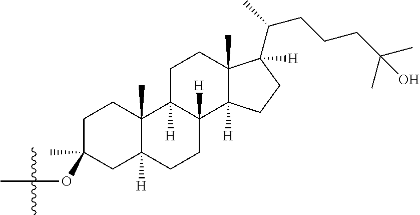 |
| 845 | 1 | 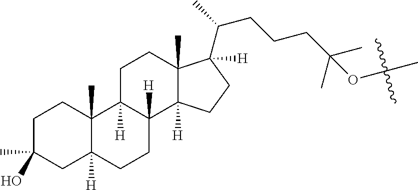 |
| 846 | 2 | 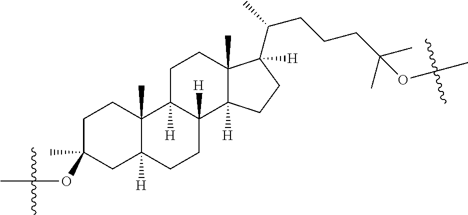 |
| 847 | 1 | 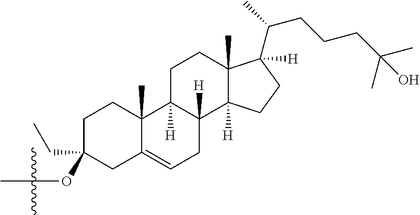 |
| 848 | 1 | 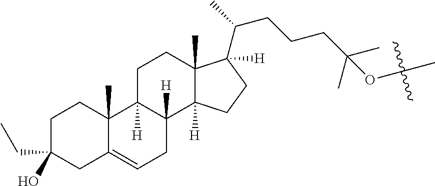 |
| 849 | 2 | 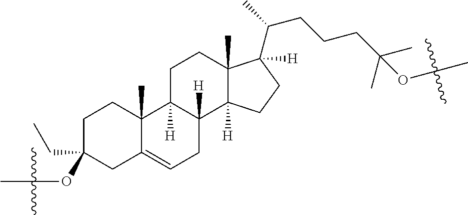 |
| 850 | 1 | 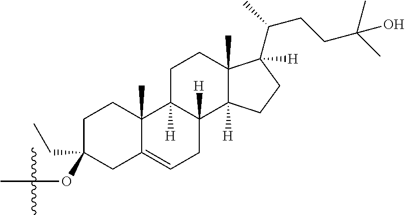 |
| 851 | 1 | 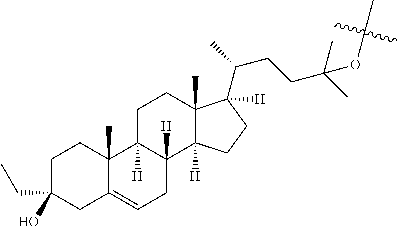 |
| 852 | 2 | 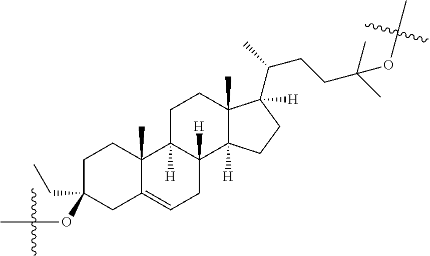 |

In some embodiments, the compound is selected from any of the compounds depicted in in Table 14, pharmaceutically acceptable salts thereof, deuterated variants thereof or combinations thereof.

TABLE 14

| | |
|---|---|
| | Formula (XL) |
| 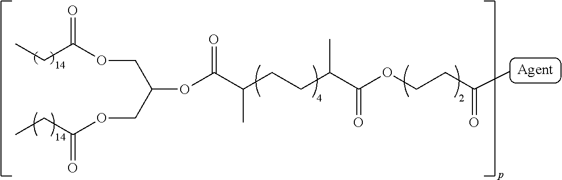 | |

| Cpd. No. | p | 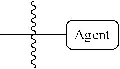 |
|---|---|---|
| 853 | 1 | 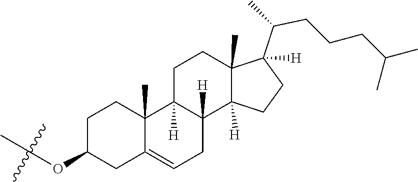 |
| 854 | 1 | 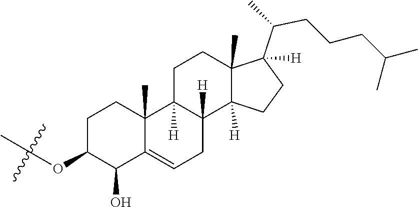 |
| 855 | 1 | 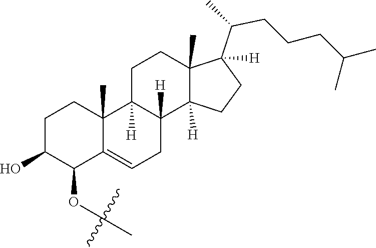 |
| 856 | 2 | 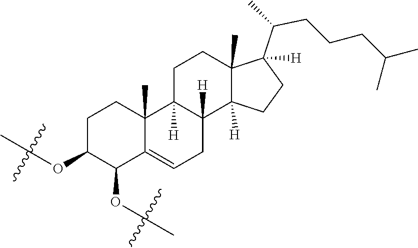 |
| 857 | 1 | 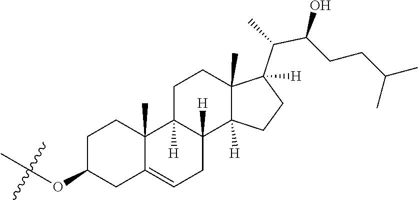 |
| 858 | 1 | 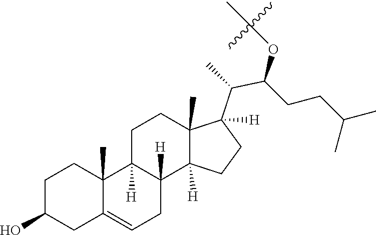 |
| 859 | 2 | 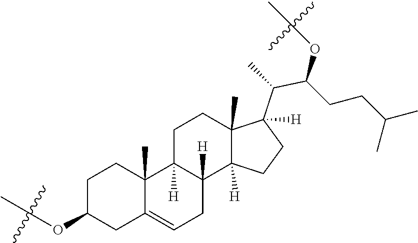 |
| 860 | 1 | 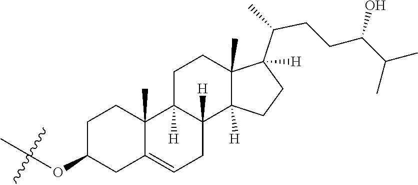 |
| 861 | 1 | 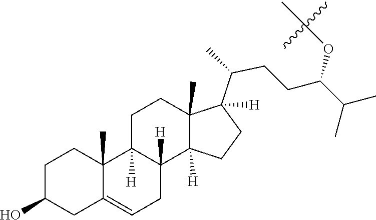 |
| 862 | 2 | 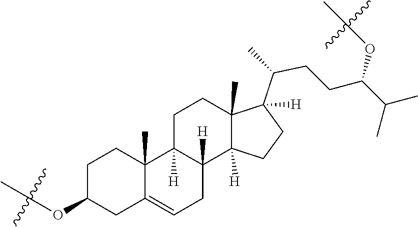 |
| 863 | 1 | 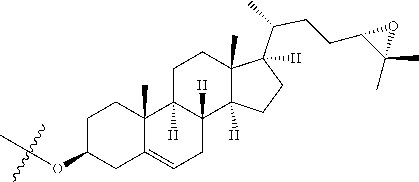 |
| 864 | 1 | 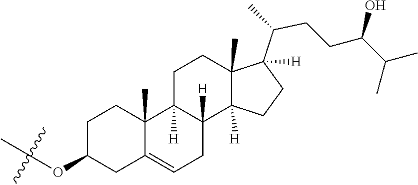 |
| 865 | 1 | 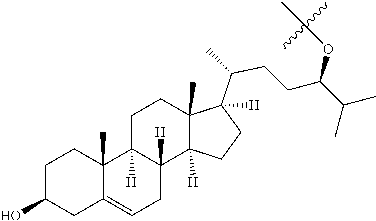 |
| 866 | 2 | 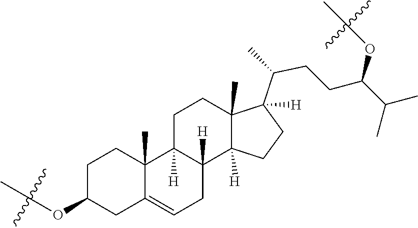 |
| 867 | 1 | 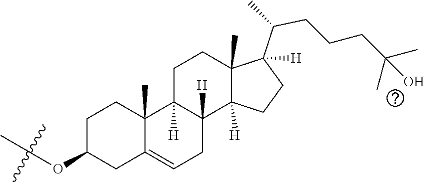 |
| 868 | 1 | 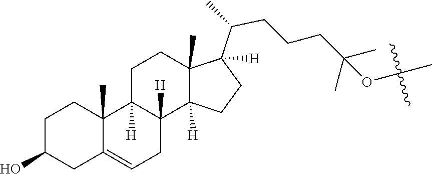 |
| 869 | 2 | 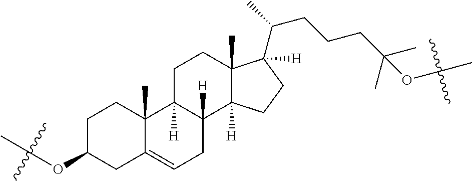 |
| 870 | 1 | 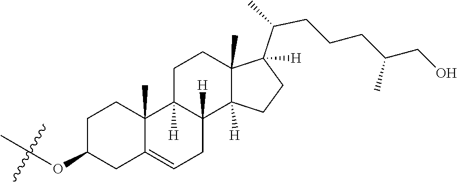 |
| 871 | 1 | 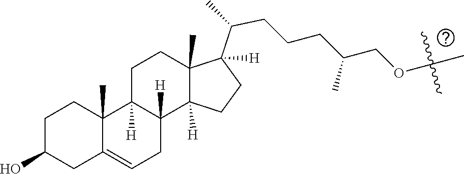 |
| 872 | 2 | 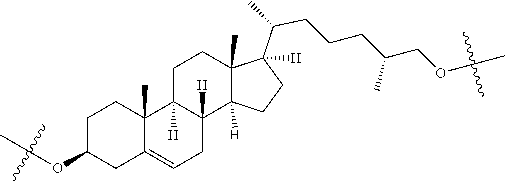 |
| 873 | 1 | 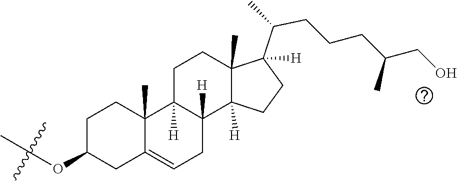 |
| 874 | 1 | 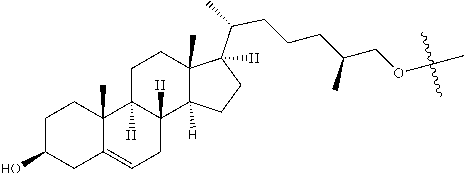 |
| 875 | 2 | 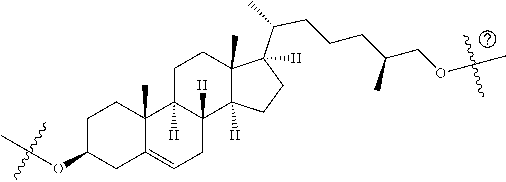 |
| 876 | 1 | 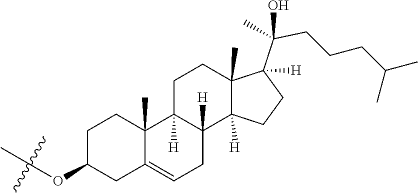 |
| 877 | 1 | 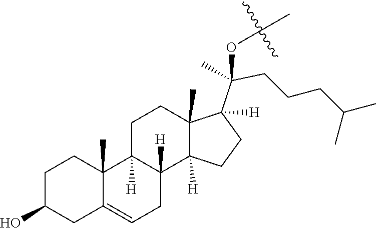 |
| 878 | 2 | 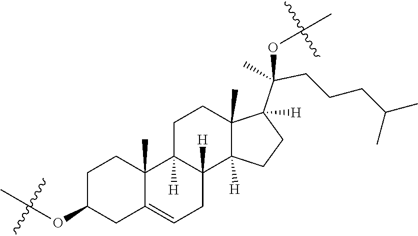 |
| 879 | 1 | 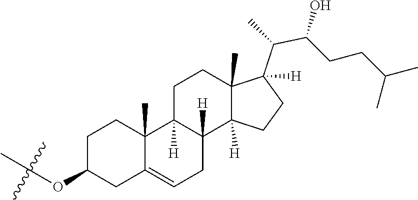 |
| 880 | 1 | 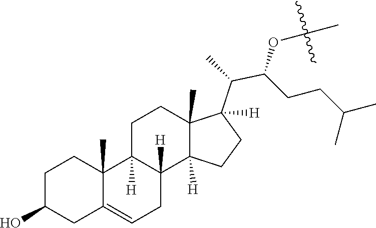 |
| 881 | 2 | 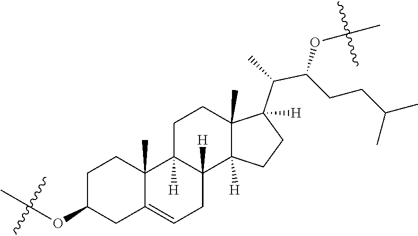 |
| 882 | 1 | 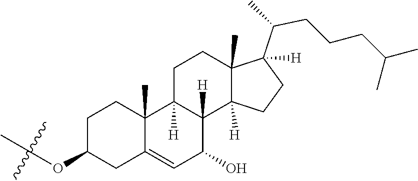 |
| 883 | 1 | 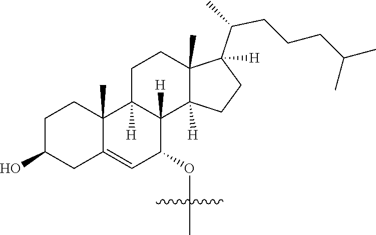 |
| 884 | 2 | 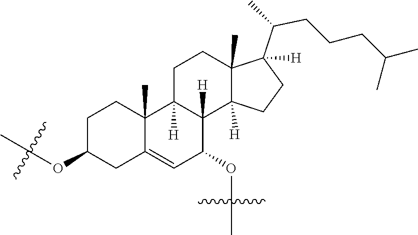 |
| 885 | 1 | 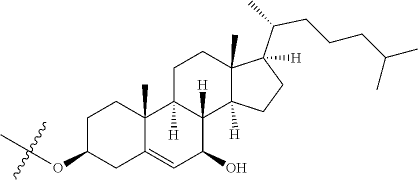 |
| 886 | 1 | 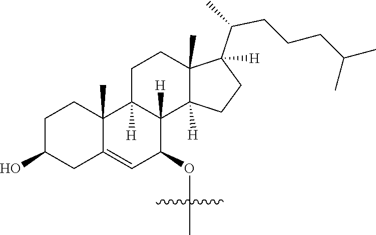 |
| 887 | 2 | 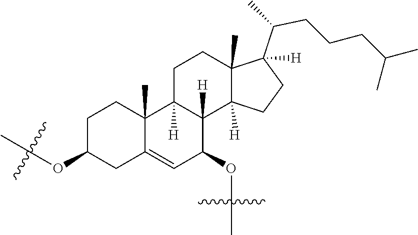 |
| 888 | 1 | 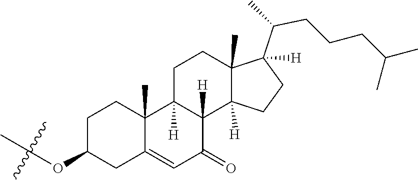 |
| 889 | 1 | 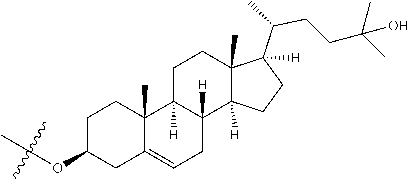 |
| 890 | 1 | 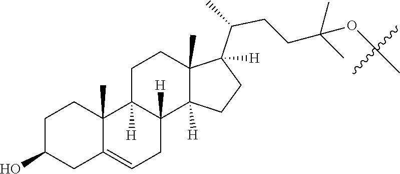 |
| 891 | 2 | 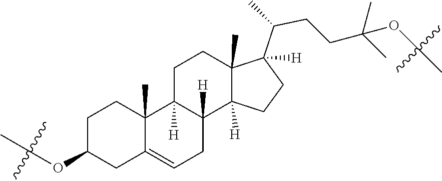 |
| 892 | 1 | 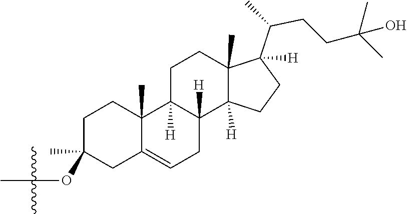 |
| 893 | 1 | 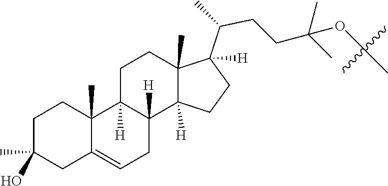 |
| 894 | 2 | 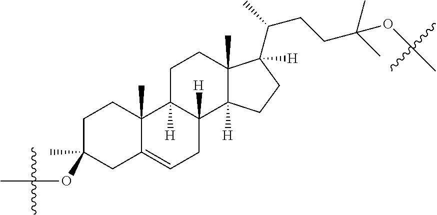 |
| 895 | 1 | 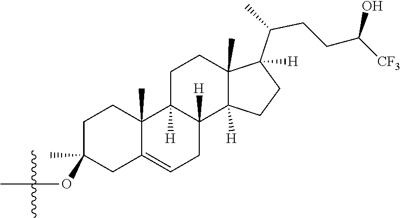 |
| 896 | 1 | 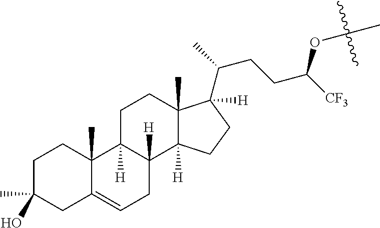 |
| 897 | 2 | 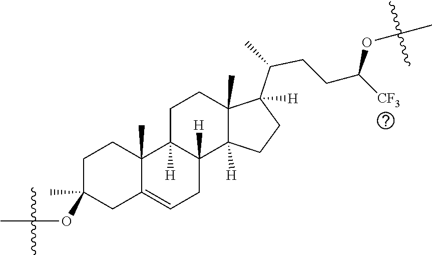 |
| 898 | 1 | 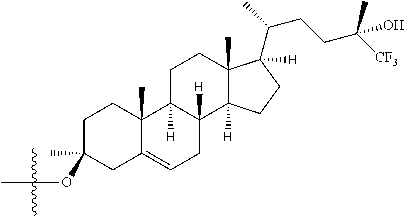 |
| 899 | 1 | 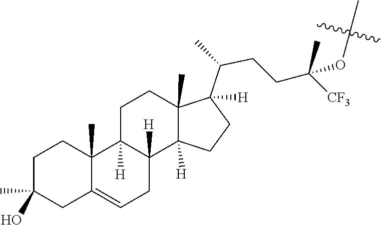 |
| 900 | 2 | 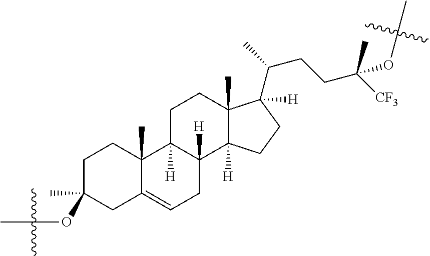 |
| 901 | 1 | 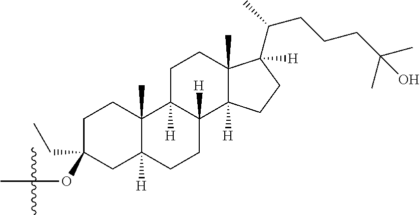 |
| 902 | 1 | 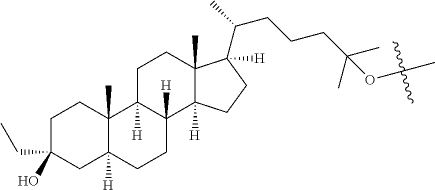 |
| 903 | 2 | 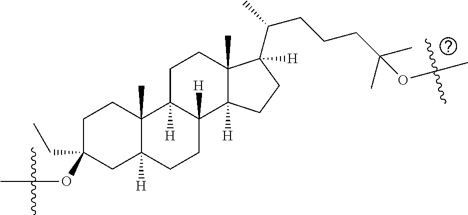 |
| 904 | 1 | 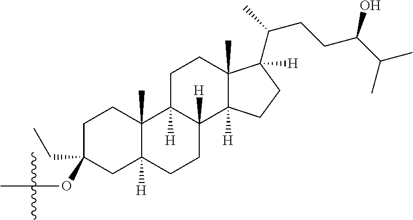 |
| 905 | 1 | 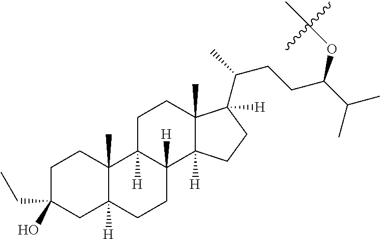 |
| 906 | 2 | 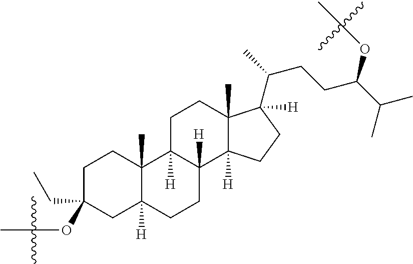 |
| 907 | 1 | 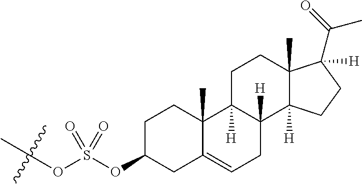 |
| 908 | 1 | 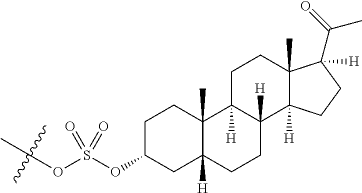 |
| 909 | 1 | 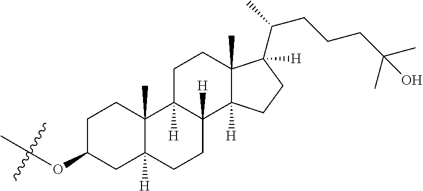 |
| 910 | 1 | 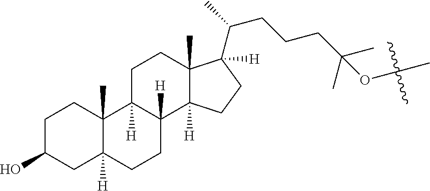 |
| 911 | 2 | 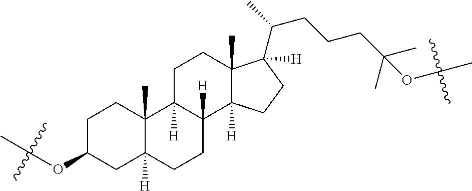 |
| 912 | 1 | 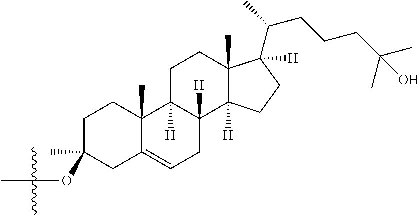 |
| 913 | 1 | 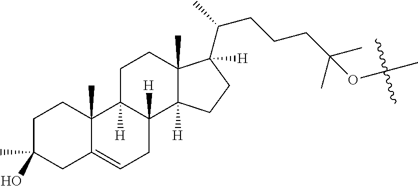 |
| 914 | 2 | 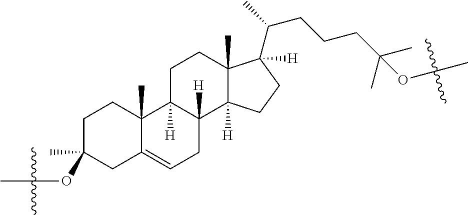 |
| 915 | 1 | 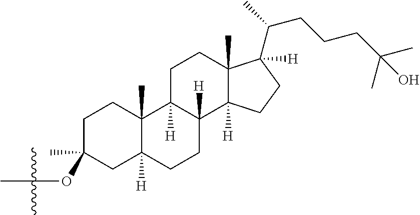 |
| 916 | 1 | 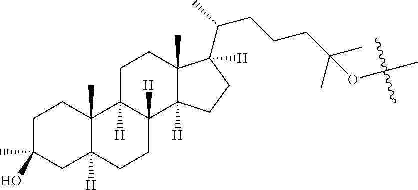 |
| 917 | 2 | 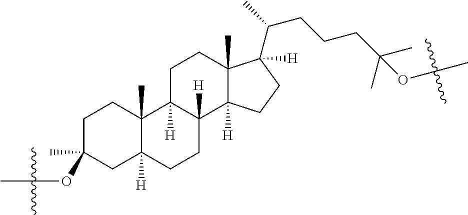 |
| 918 | 1 | 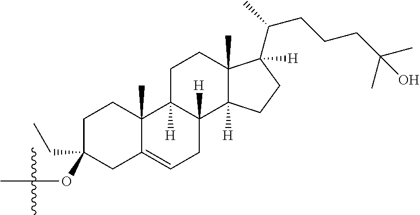 |
| 919 | 1 | 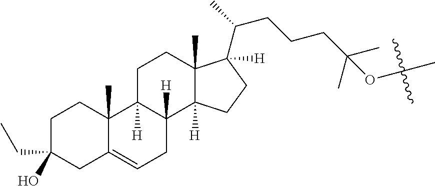 |
| 920 | 2 | 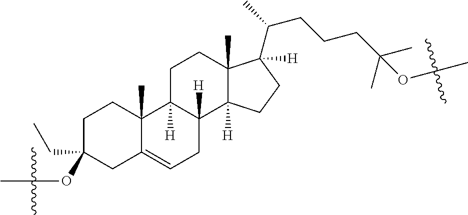 |
| 921 | 1 | 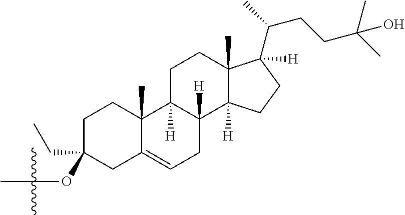 |
| 922 | 1 | 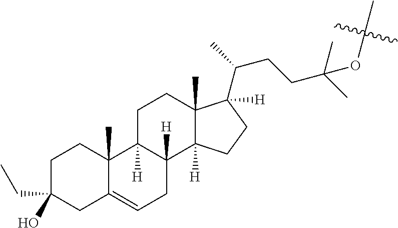 |
| 923 | 2 | 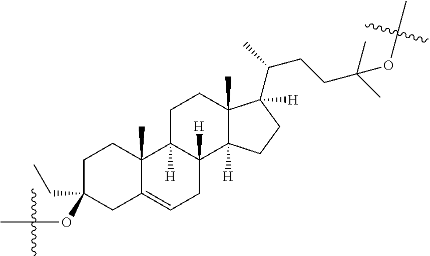 |

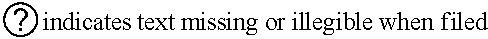

In some embodiments, the compound is selected from any of the compounds depicted in in Table 15, pharmaceutically acceptable salts thereof, deuterated variants thereof or combinations thereof.

TABLE 15

| | |
|---|---|
| | Formula (XLI) |
| 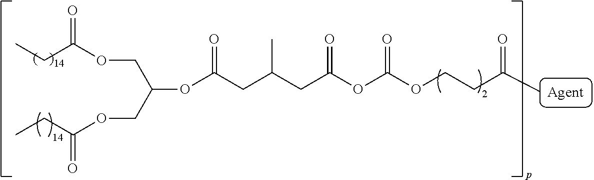 | |

| Cpd. No. | p | 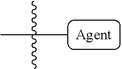 |
|---|---|---|
| 924 | 1 | 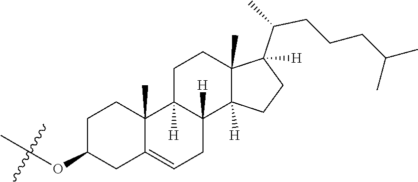 |
| 925 | 1 | 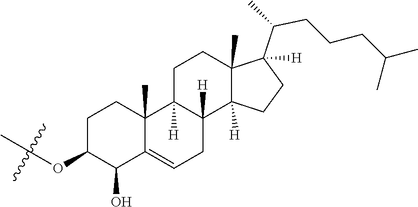 |
| 926 | 1 | 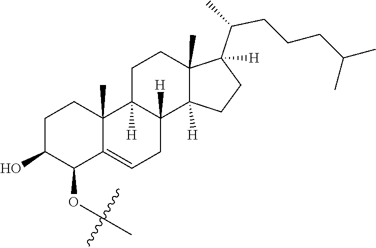 |
| 927 | 2 | 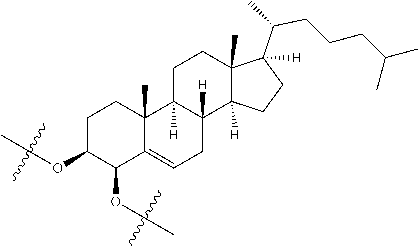 |
| 928 | 1 | 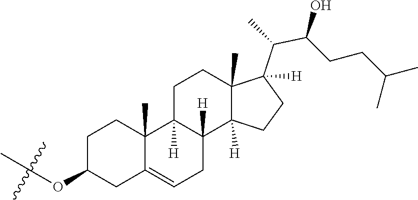 |
| 929 | 1 | 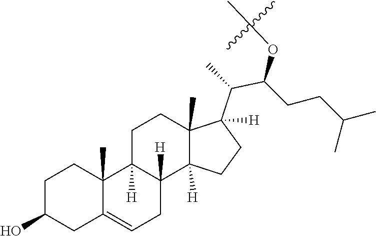 |
| 930 | 2 | 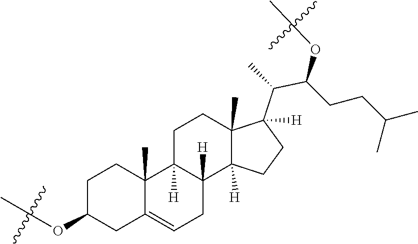 |
| 931 | 1 | 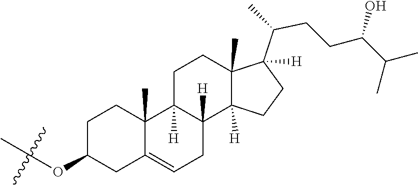 |
| 932 | 1 | 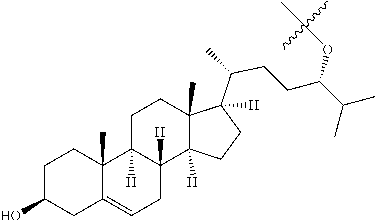 |
| 933 | 2 | 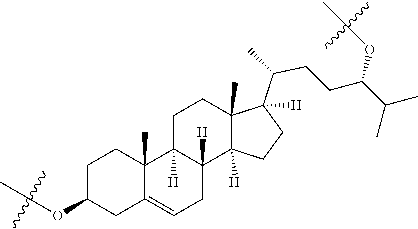 |
| 934 | 1 | 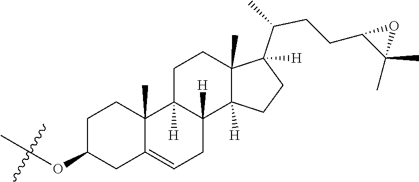 |
| 935 | 1 | 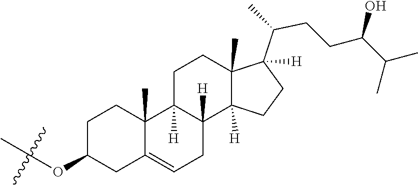 |
| 936 | 1 | 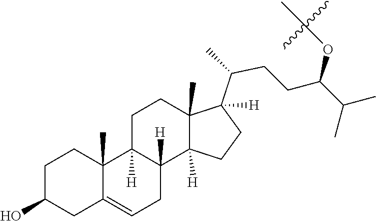 |
| 937 | 2 | 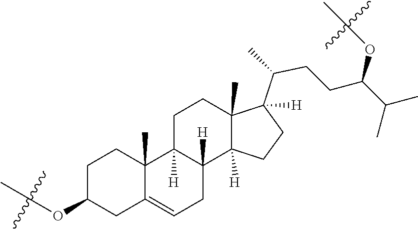 |
| 938 | 1 | 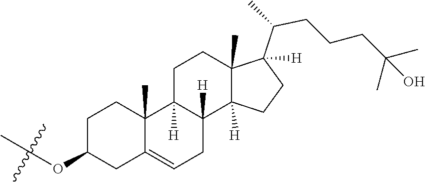 |
| 939 | 1 | 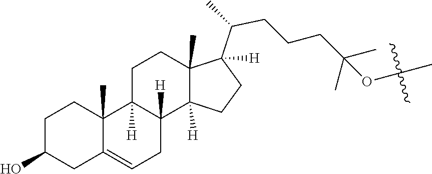 |
| 940 | 2 | 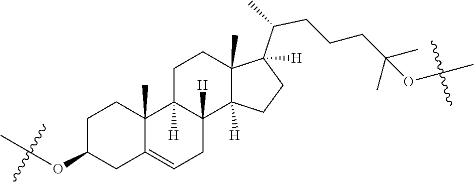 |
| 941 | 1 | 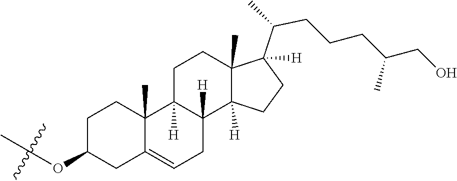 |
| 942 | 1 | 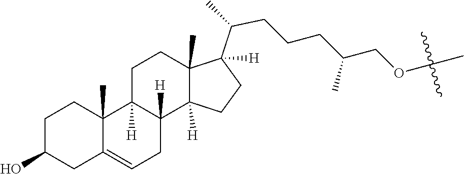 |
| 943 | 2 | 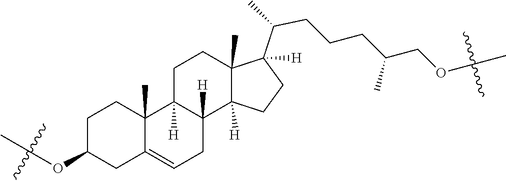 |
| 944 | 1 | 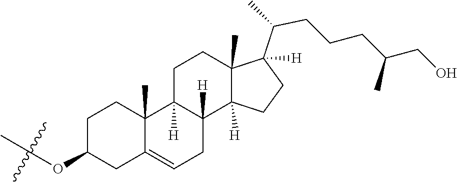 |
| 945 | 1 | 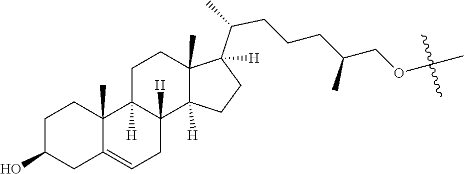 |
| 946 | 2 | 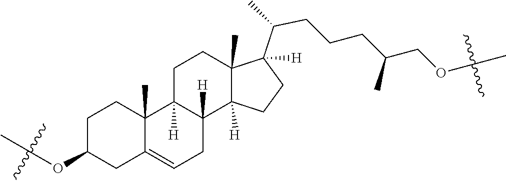 |
| 947 | 1 | 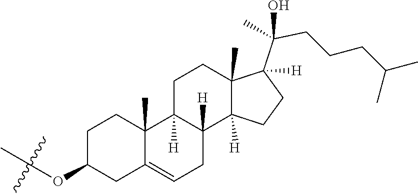 |
| 948 | 1 | 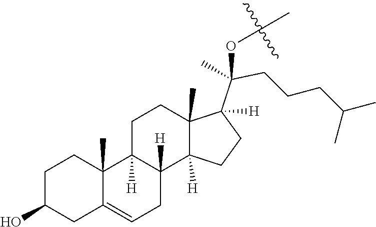 |
| 949 | 2 | 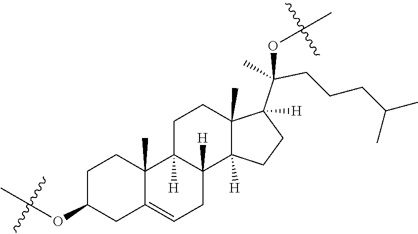 |
| 950 | 1 | 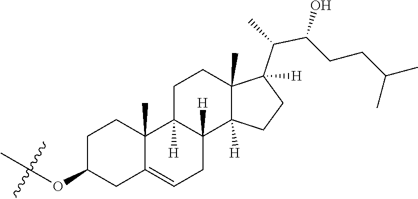 |
| 951 | 1 | 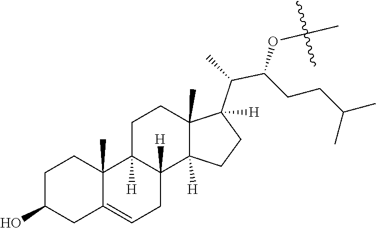 |
| 952 | 2 | 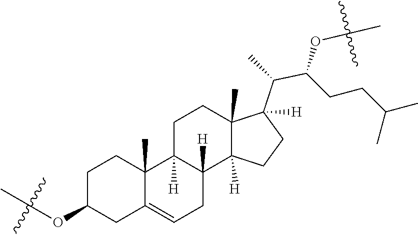 |
| 953 | 1 | 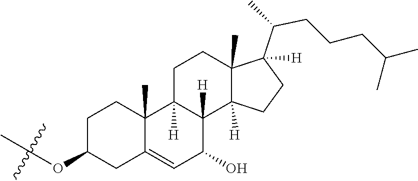 |
| 954 | 1 | 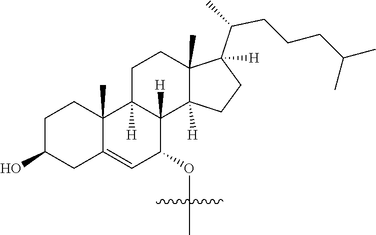 |
| 955 | 2 | 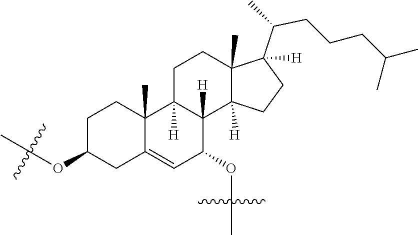 |
| 956 | 1 | 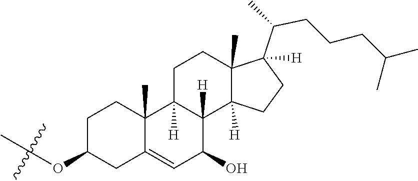 |
| 957 | 1 | 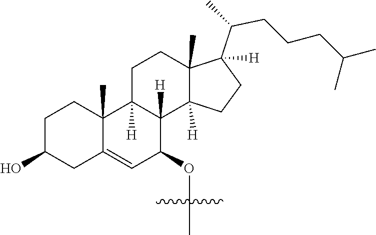 |
| 958 | 2 | 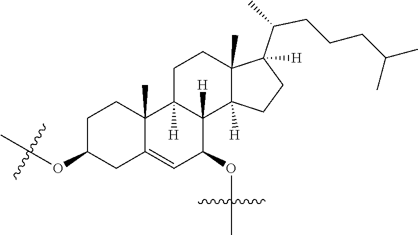 |
| 959 | 1 | 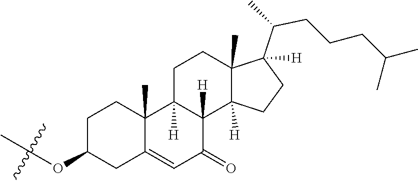 |
| 960 | 1 | 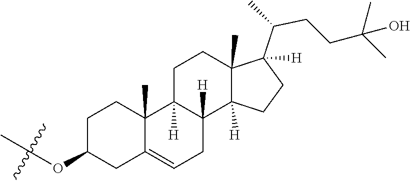 |
| 961 | 1 | 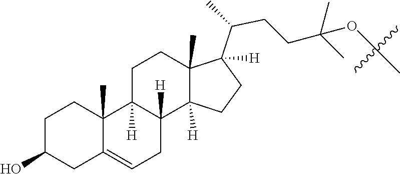 |
| 962 | 2 | 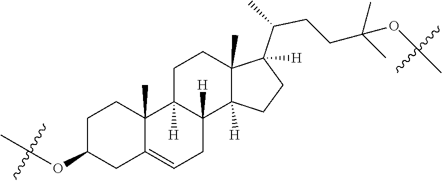 |
| 963 | 1 | 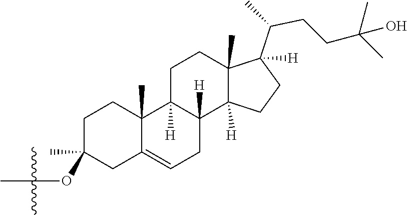 |
| 964 | 1 | 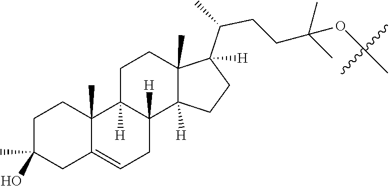 |
| 965 | 2 | 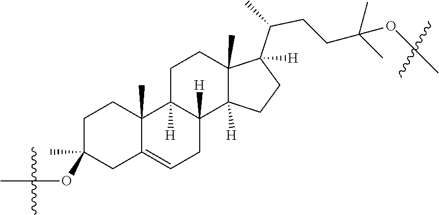 |
| 966 | 1 | 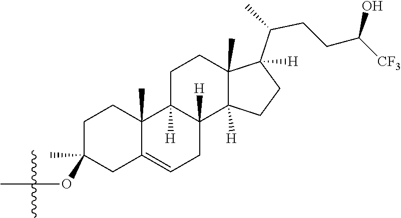 |
| 967 | 1 | 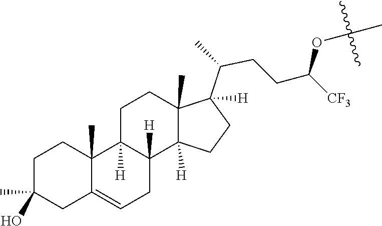 |
| 968 | 2 | 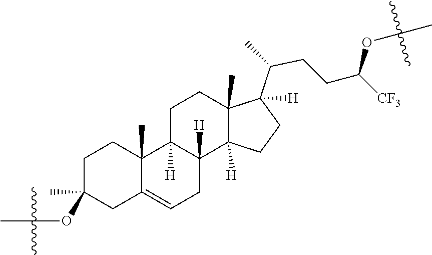 |
| 969 | 1 | 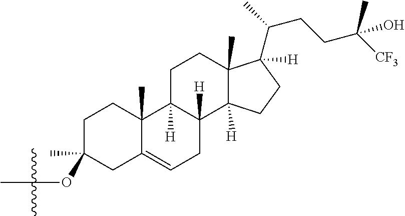 |
| 970 | 1 | 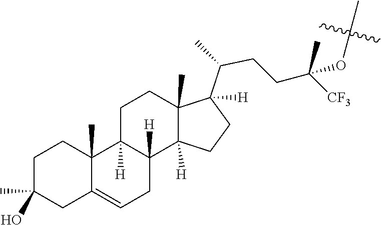 |
| 971 | 2 | 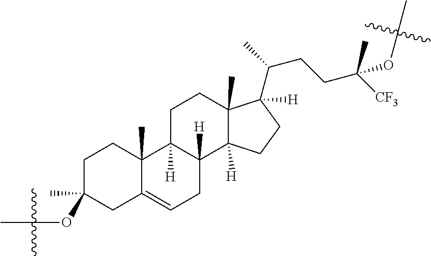 |
| 972 | 1 | 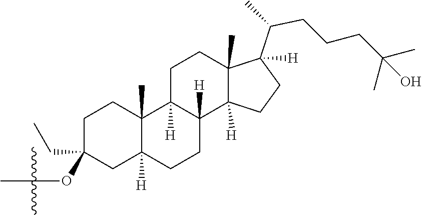 |
| 973 | 1 | 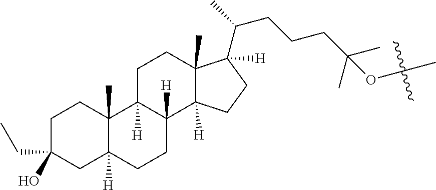 |
| 974 | 2 | 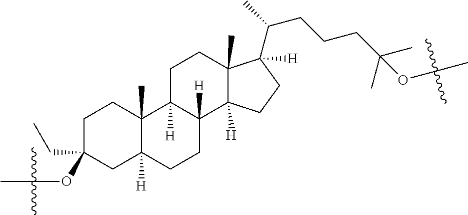 |
| 975 | 1 | 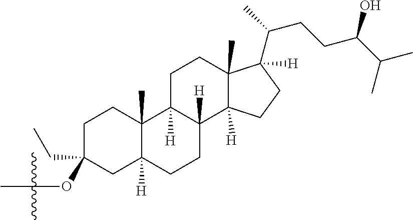 |
| 976 | 1 | 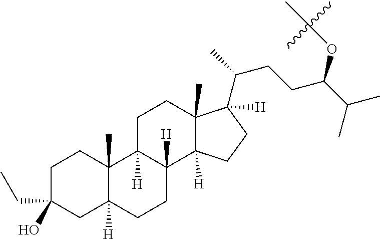 |
| 977 | 2 | 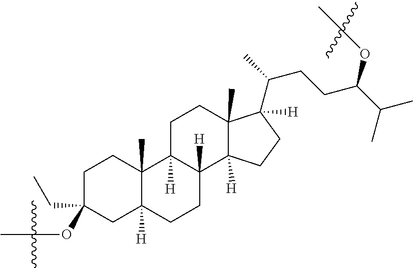 |
| 978 | 1 | 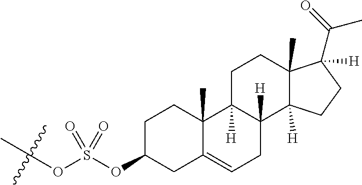 |
| 979 | 1 | 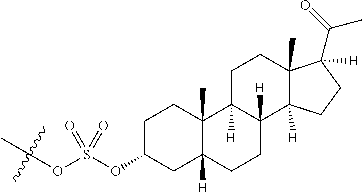 |
| 980 | 1 | 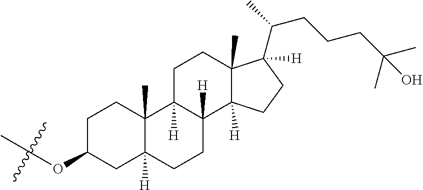 |
| 981 | 1 | 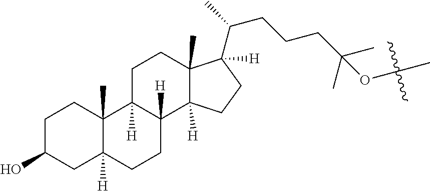 |
| 982 | 2 | 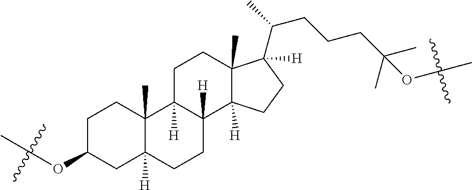 |
| 983 | 1 | 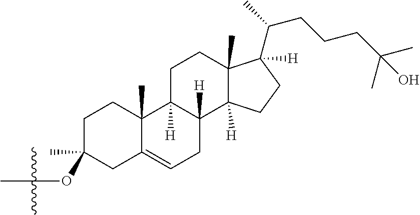 |
| 984 | 1 | 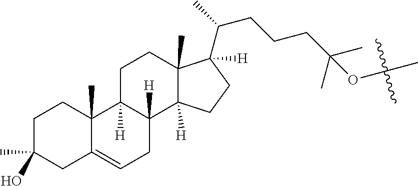 |
| 985 | 2 | 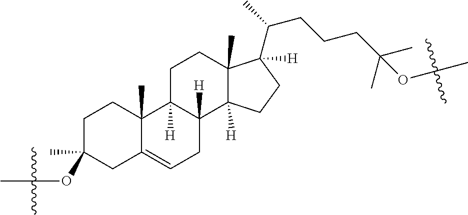 |
| 986 | 1 | 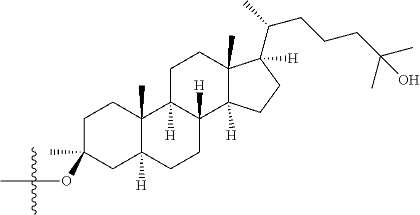 |
| 987 | 1 | 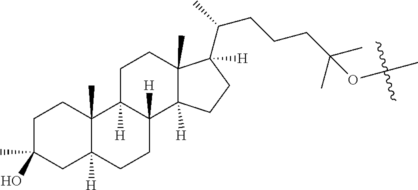 |
| 988 | 2 | 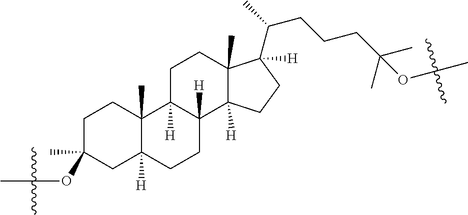 |
| 989 | 1 | 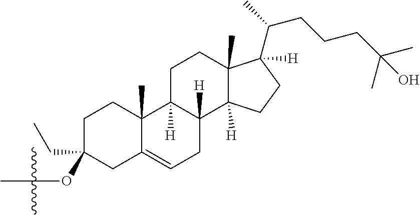 |
| 990 | 1 | 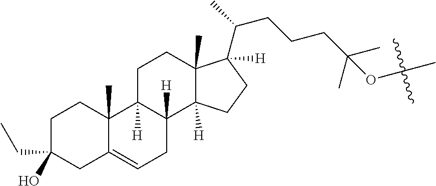 |
| 991 | 2 | 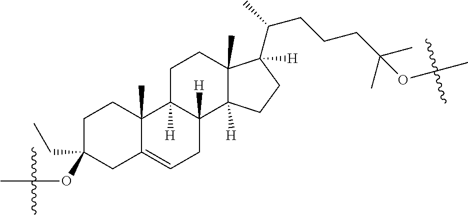 |
| 992 | 1 | 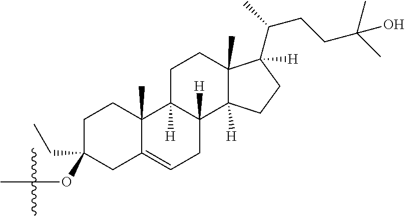 |
| 993 | 1 | 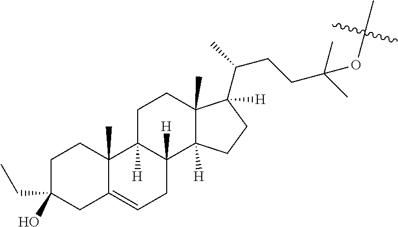 |
| 994 | 2 | 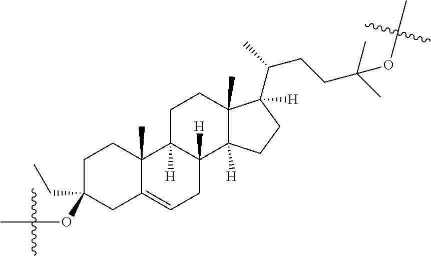 |

In some embodiments, the compound is selected from any of the compounds depicted in in Table 16, pharmaceutically acceptable salts thereof, deuterated variants thereof or combinations thereof.

TABLE 16

| | |
|---|---|
| | Formula (XLII) |
| 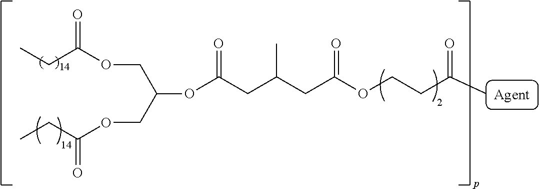 | |

| Cpd. No. | p | 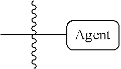 |
|---|---|---|
| 995 | 1 | 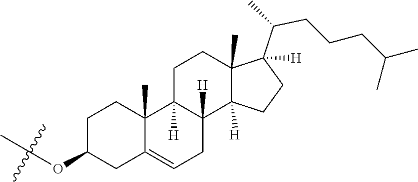 |
| 996 | 1 | 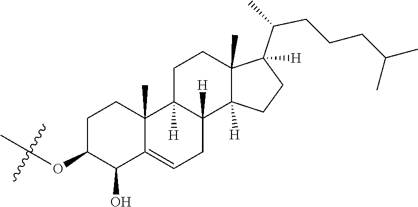 |
| 997 | 1 | 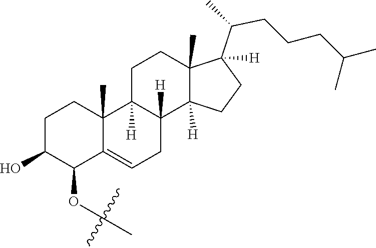 |
| 998 | 2 | 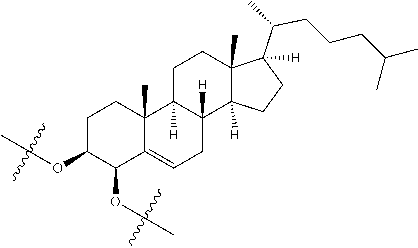 |
| 999 | 1 | 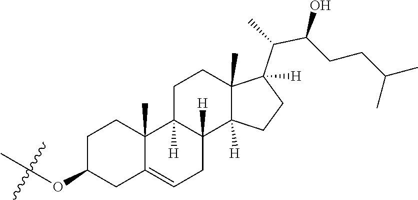 |
| 1000 | 1 | 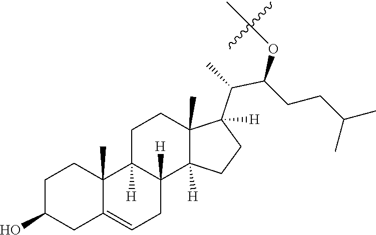 |
| 1001 | 2 | 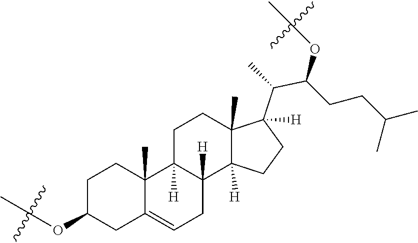 |
| 1002 | 1 | 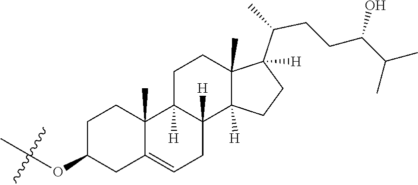 |
| 1003 | 1 | 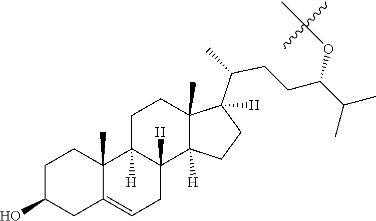 |
| 1004 | 2 | 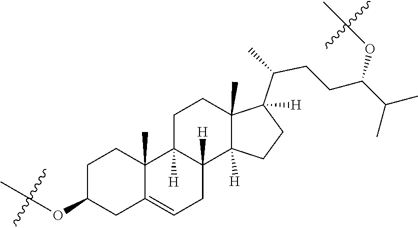 |
| 1005 | 1 | 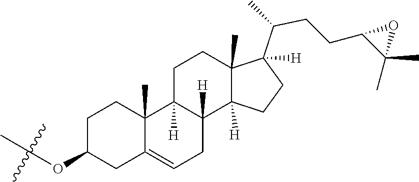 |
| 1006 | 1 | 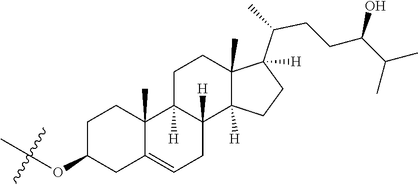 |
| 1007 | 1 | 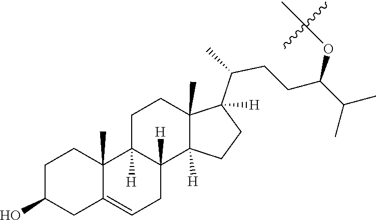 |
| 1008 | 2 | 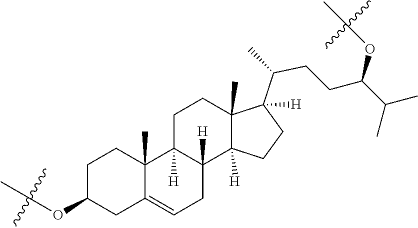 |
| 1009 | 1 | 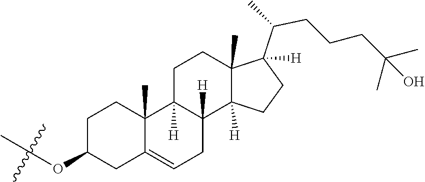 |
| 1010 | 1 | 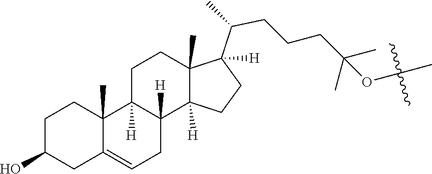 |
| 1011 | 2 | 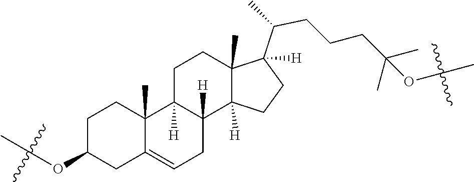 |
| 1012 | 1 | 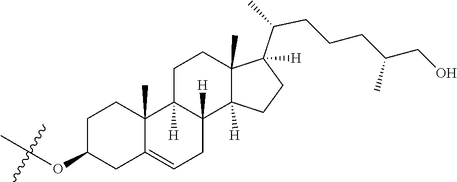 |
| 1013 | 1 | 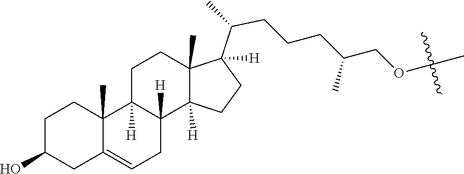 |
| 1014 | 2 | 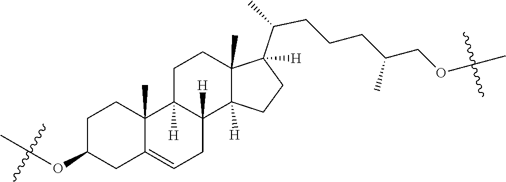 |
| 1015 | 1 | 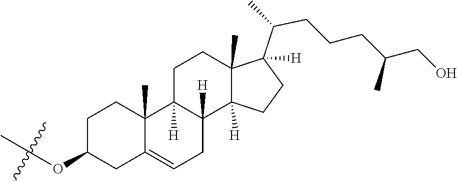 |
| 1016 | 1 | 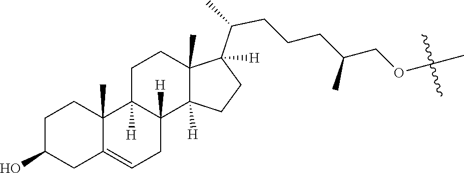 |
| 1017 | 2 | 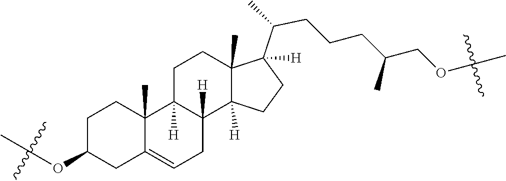 |
| 1018 | 1 | 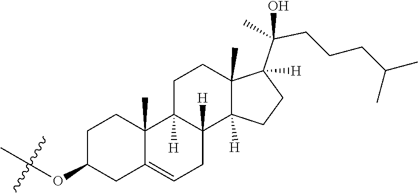 |
| 1019 | 1 | 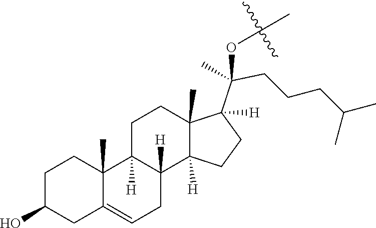 |
| 1020 | 2 | 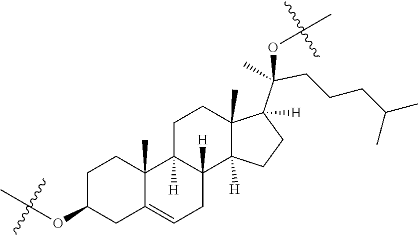 |
| 1021 | 1 | 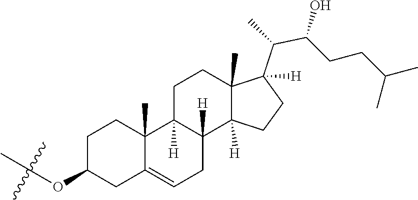 |
| 1022 | 1 | 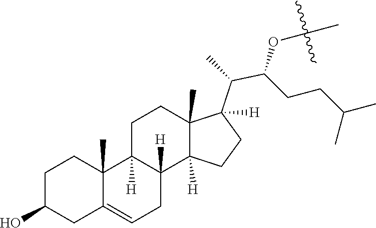 |
| 1023 | 2 | 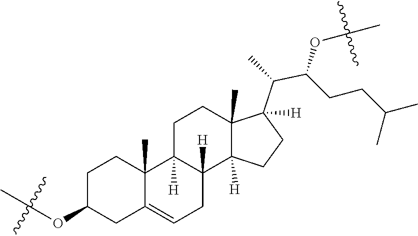 |
| 1024 | 1 | 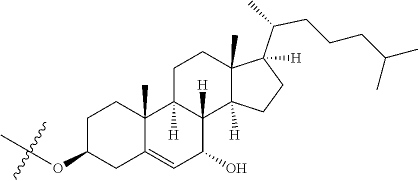 |
| 1025 | 1 | 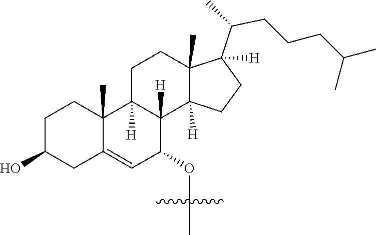 |
| 1026 | 2 | 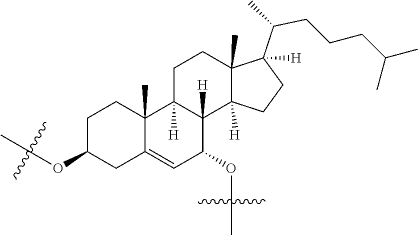 |
| 1027 | 1 | 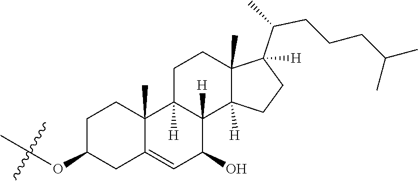 |
| 1028 | 1 | 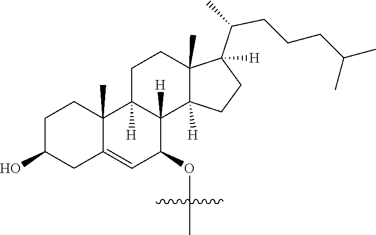 |
| 1029 | 2 | 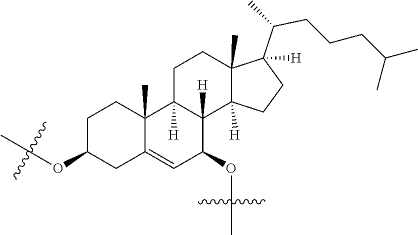 |
| 1030 | 1 | 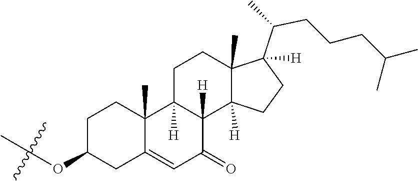 |
| 1031 | 1 | 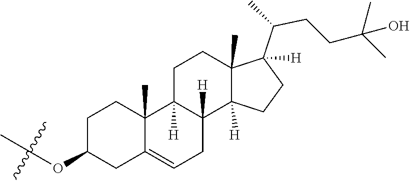 |
| 1032 | 1 | 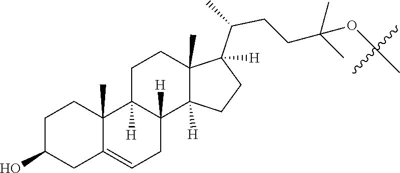 |
| 1033 | 2 | 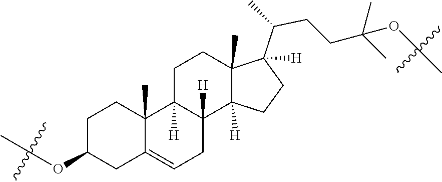 |
| 1034 | 1 | 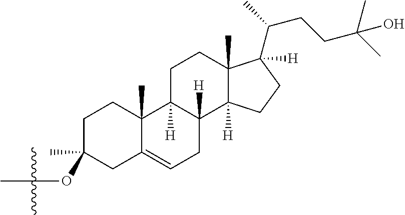 |
| 1035 | 1 | 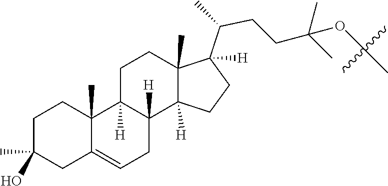 |
| 1036 | 2 | 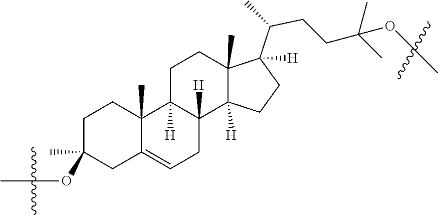 |
| 1037 | 1 | 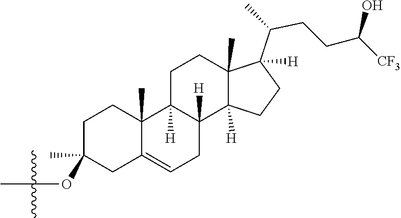 |
| 1038 | 1 | 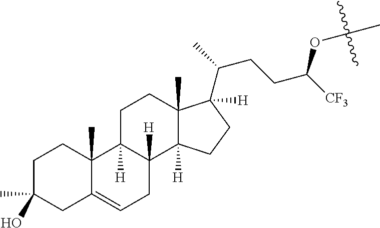 |
| 1039 | 2 | 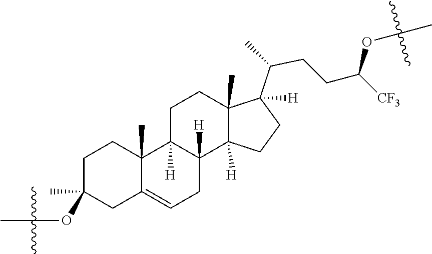 |
| 1040 | 1 | 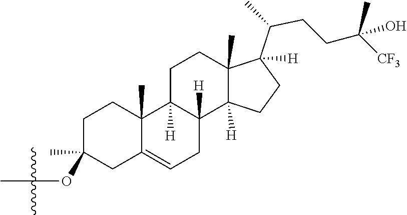 |
| 1041 | 1 | 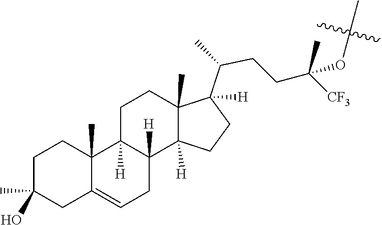 |
| 1042 | 2 | 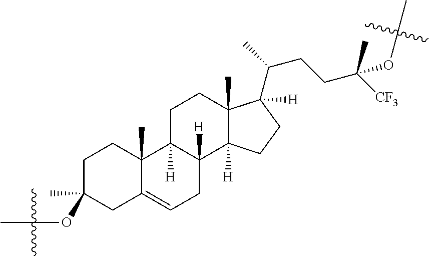 |
| 1043 | 1 | 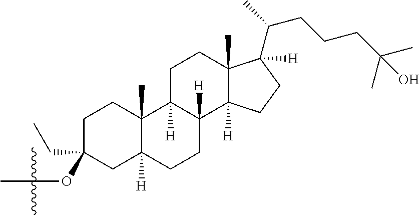 |
| 1044 | 1 | 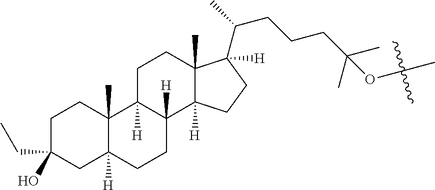 |
| 1045 | 2 | 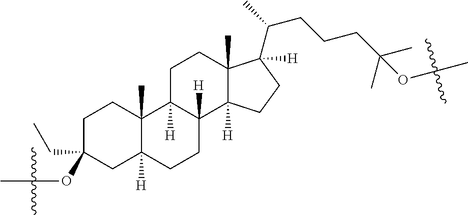 |
| 1046 | 1 | 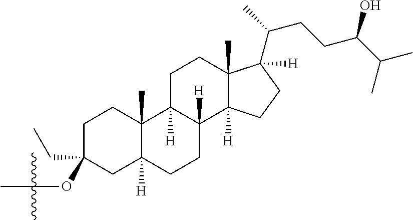 |
| 1047 | 1 | 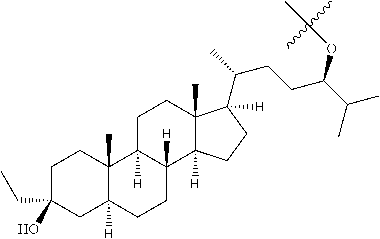 |
| 1048 | 2 | 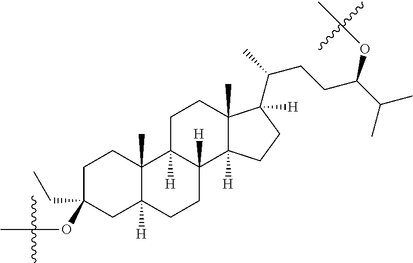 |
| 1049 | 1 | 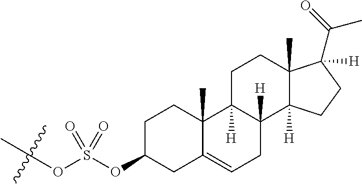 |
| 1050 | 1 | 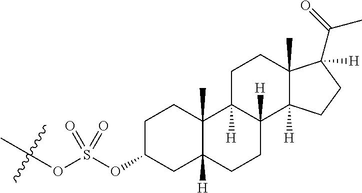 |
| 1051 | 1 | 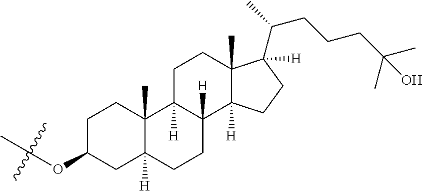 |
| 1052 | 1 | 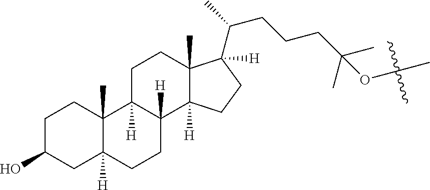 |
| 1053 | 2 | 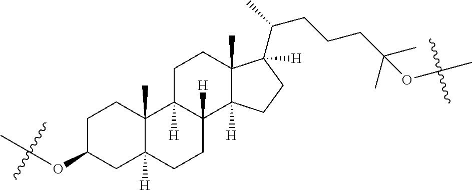 |
| 1054 | 1 | 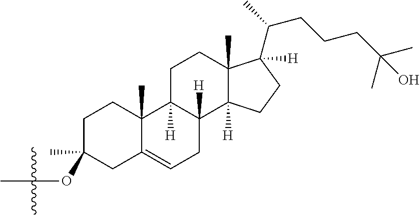 |
| 1055 | 1 | 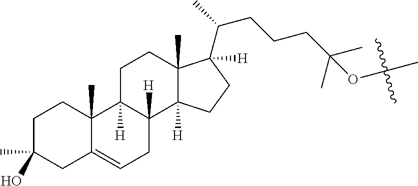 |
| 1056 | 2 | 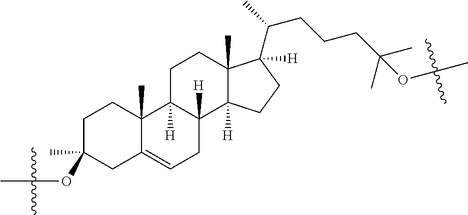 |
| 1057 | 1 | 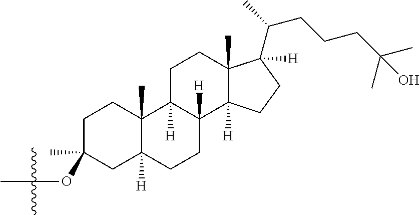 |
| 1058 | 1 | 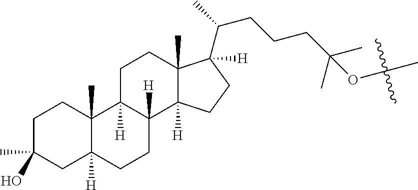 |
| 1059 | 2 | 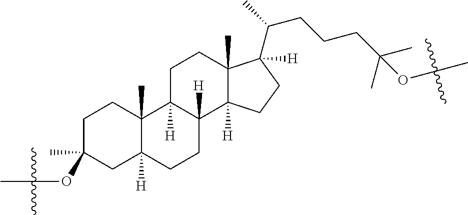 |
| 1060 | 1 | 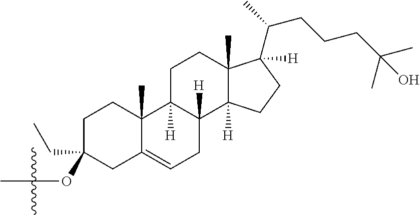 |
| 1061 | 1 | 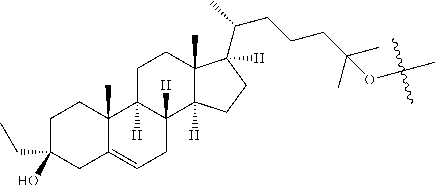 |
| 1062 | 2 | 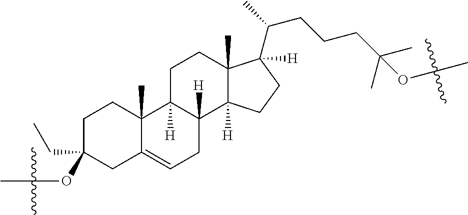 |
| 1063 | 1 | 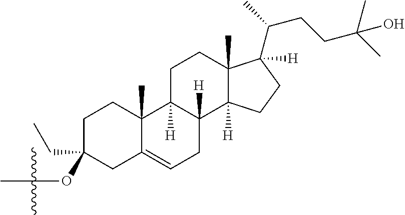 |
| 1064 | 1 | 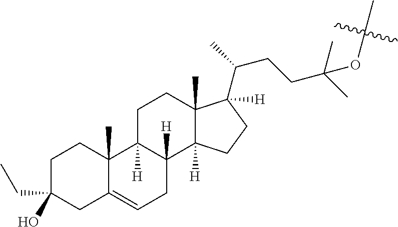 |
| 1065 | 2 | 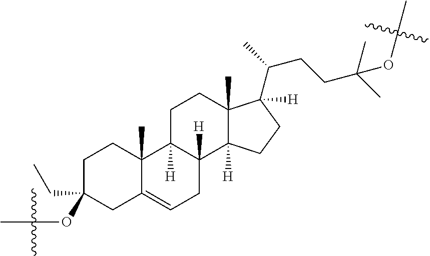 |

In some embodiments, the compound is selected from any of the compounds depicted in in Table 17, pharmaceutically acceptable salts thereof, deuterated variants thereof or combinations thereof.

TABLE 17

| | |
|---|---|
| | Formula (XLIII) |
| 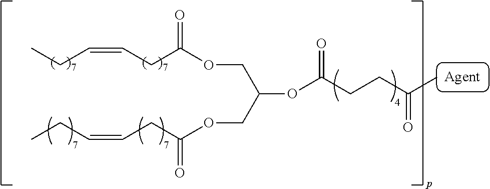 | |

| Cpd. No. | p | 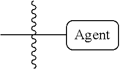 |
|---|---|---|
| 1066 | 1 | 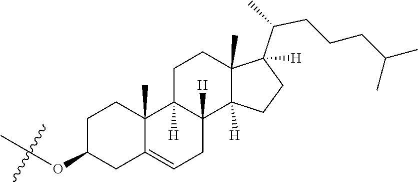 |
| 1067 | 1 | 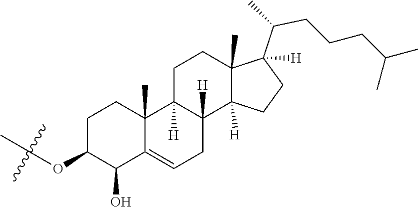 |
| 1068 | 1 | 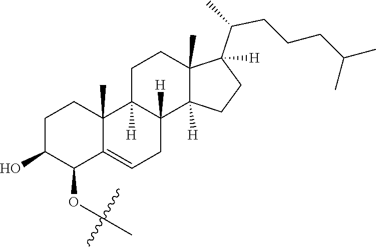 |
| 1069 | 2 | 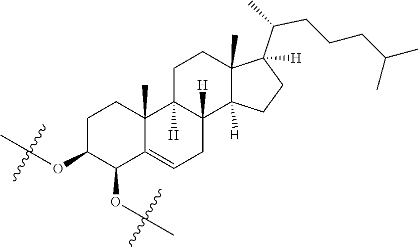 |
| 1070 | 1 | 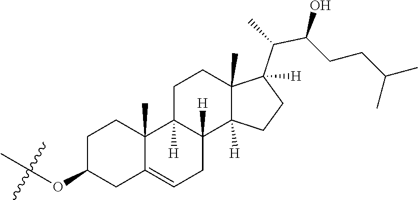 |
| 1071 | 1 | 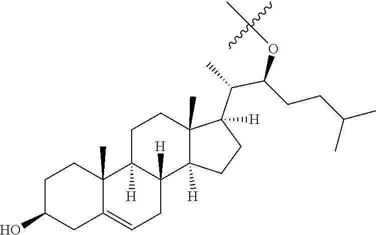 |
| 1072 | 2 | 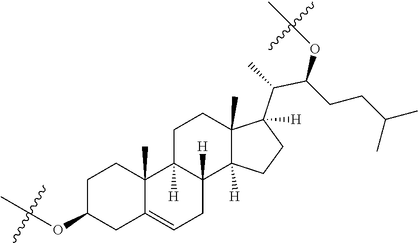 |
| 1073 | 1 | 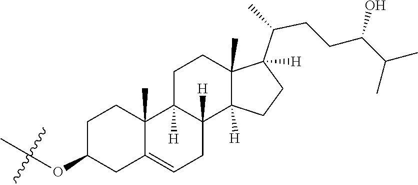 |
| 1074 | 1 | 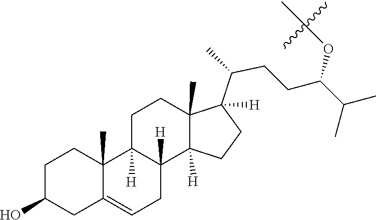 |
| 1075 | 2 | 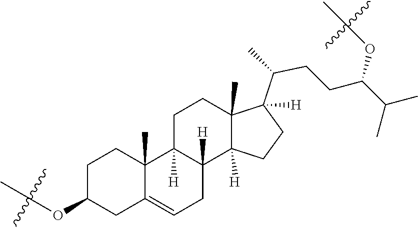 |
| 1076 | 1 | 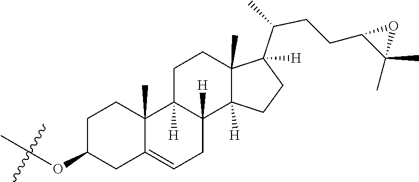 |
| 1077 | 1 | 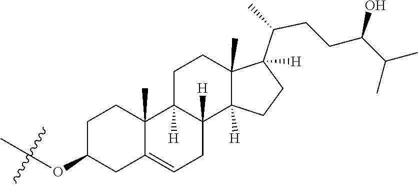 |
| 1078 | 1 | 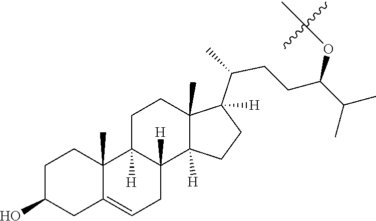 |
| 1079 | 2 | 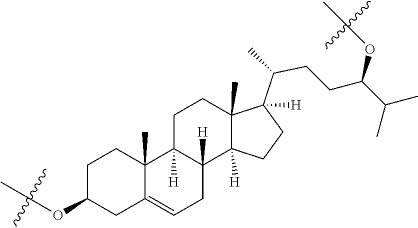 |
| 1080 | 1 | 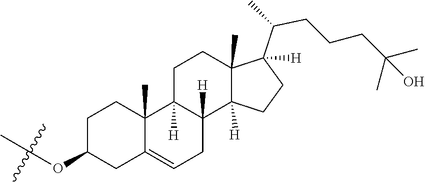 |
| 1081 | 1 | 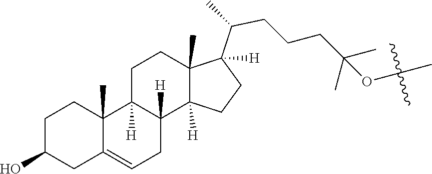 |
| 1082 | 2 | 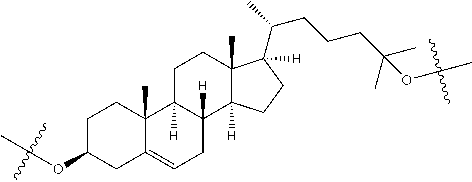 |
| 1083 | 1 | 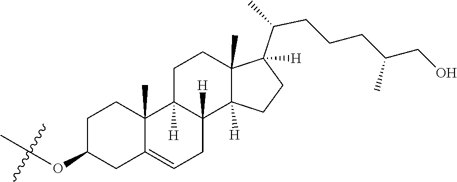 |
| 1084 | 1 | 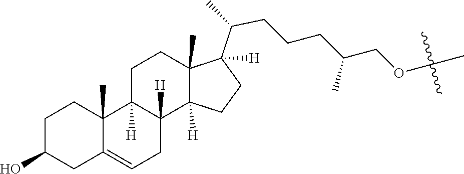 |
| 1085 | 2 | 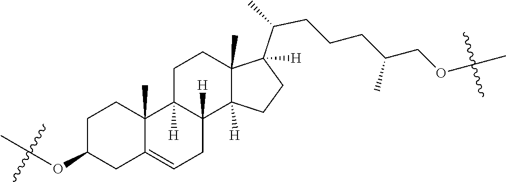 |
| 1086 | 1 | 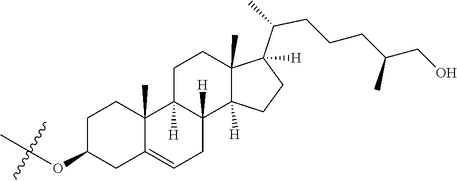 |
| 1087 | 1 | 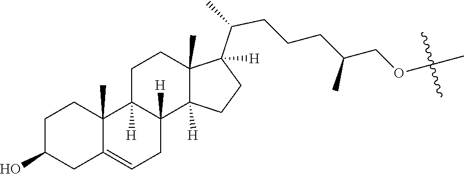 |
| 1088 | 2 | 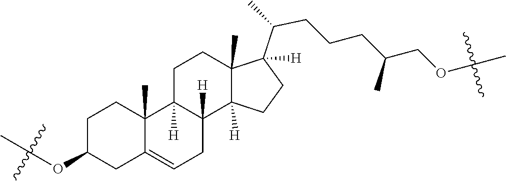 |
| 1089 | 1 | 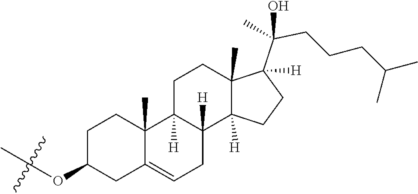 |
| 1090 | 1 | 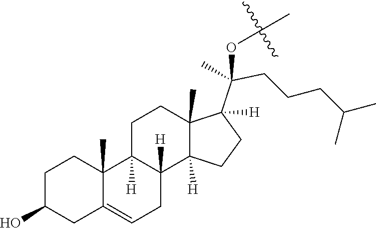 |
| 1091 | 2 | 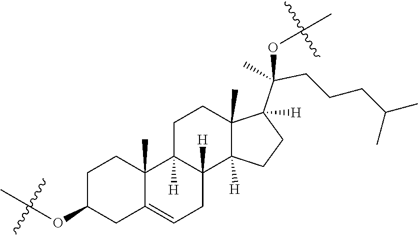 |
| 1092 | 1 | 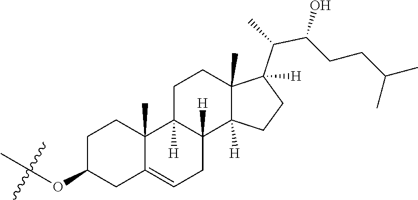 |
| 1093 | 1 | 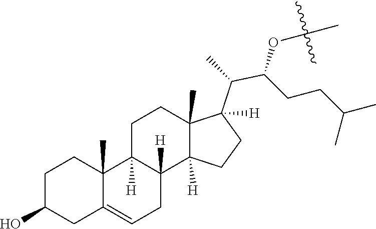 |
| 1094 | 2 | 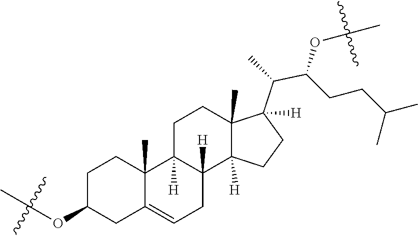 |
| 1095 | 1 | 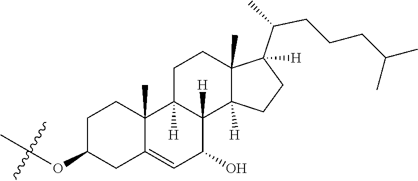 |
| 1096 | 1 | 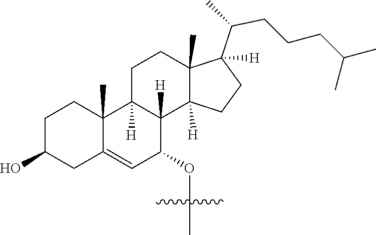 |
| 1097 | 2 | 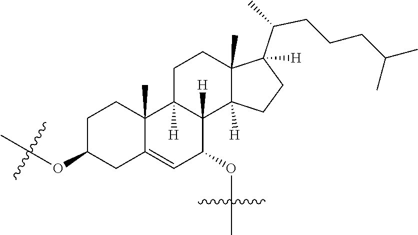 |
| 1098 | 1 | 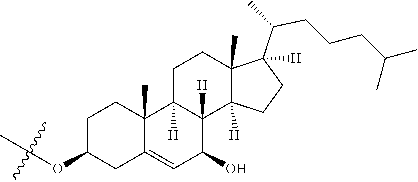 |
| 1099 | 1 | 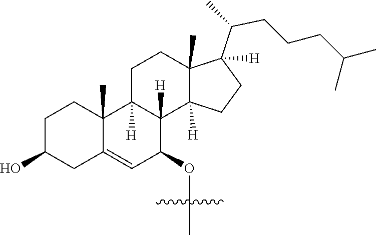 |
| 1100 | 2 | 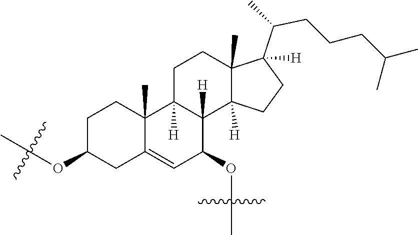 |
| 1101 | 1 | 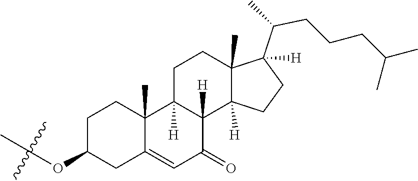 |
| 1102 | 1 | 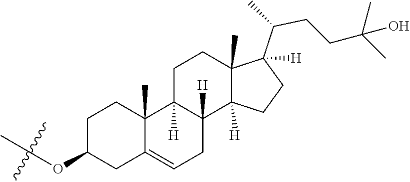 |
| 1103 | 1 | 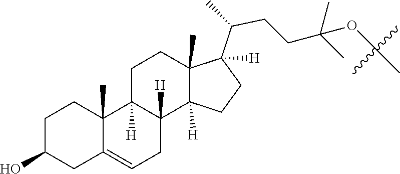 |
| 1104 | 2 | 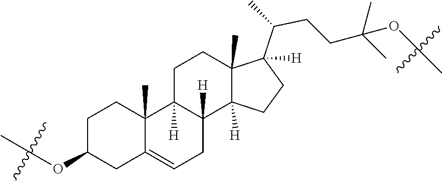 |
| 1105 | 1 | 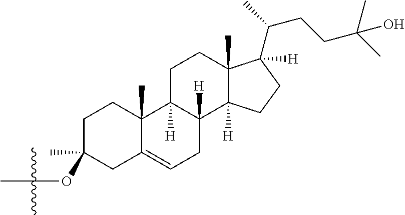 |
| 1106 | 1 | 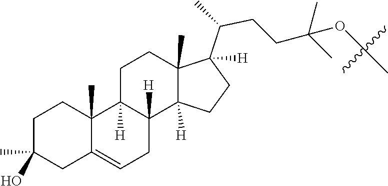 |
| 1107 | 2 | 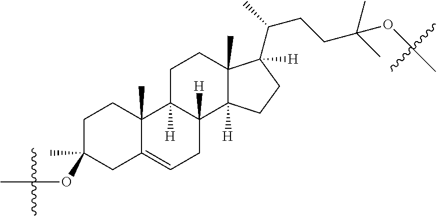 |
| 1108 | 1 | 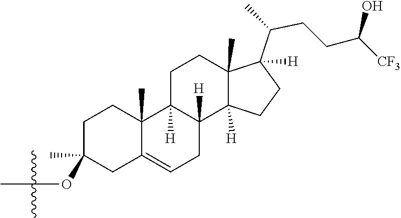 |
| 1109 | 1 | 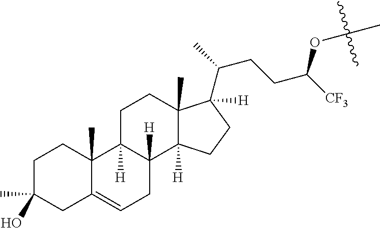 |
| 1110 | 2 | 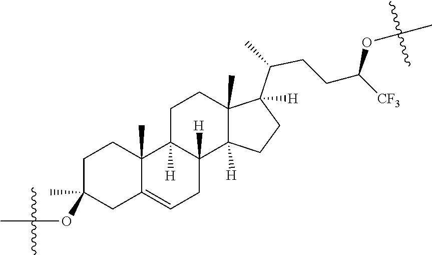 |
| 1111 | 1 | 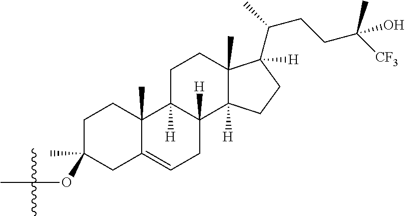 |
| 1112 | 1 | 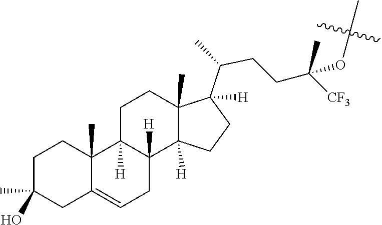 |
| 1113 | 2 | 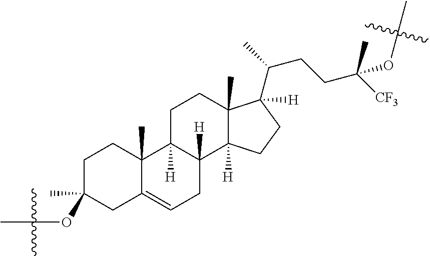 |
| 1114 | 1 | 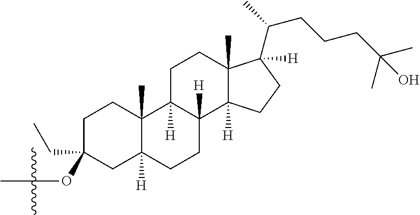 |
| 1115 | 1 | 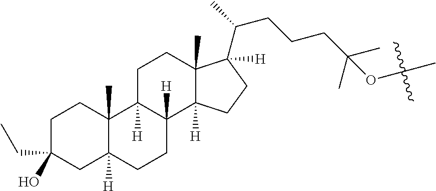 |
| 1116 | 2 | 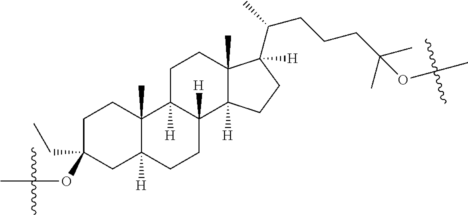 |
| 1117 | 1 | 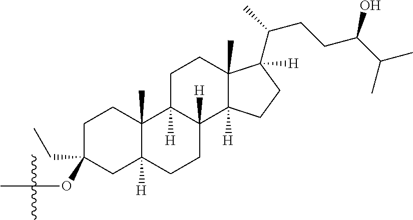 |
| 1118 | 1 | 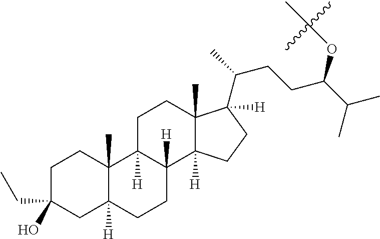 |
| 1119 | 2 | 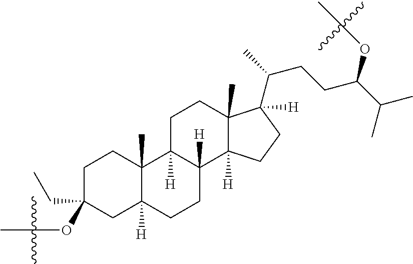 |
| 1120 | 1 | 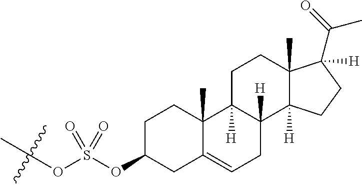 |
| 1121 | 1 | 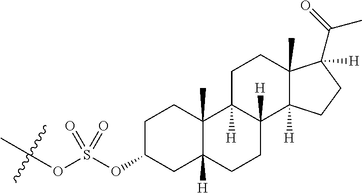 |
| 1122 | 1 | 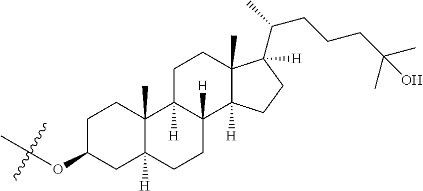 |
| 1123 | 1 | 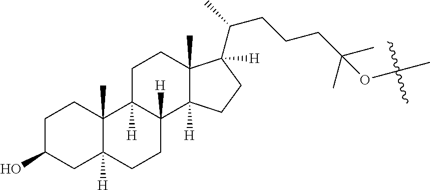 |
| 1124 | 2 | 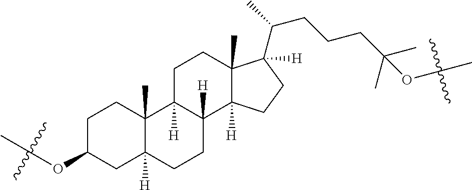 |
| 1125 | 1 | 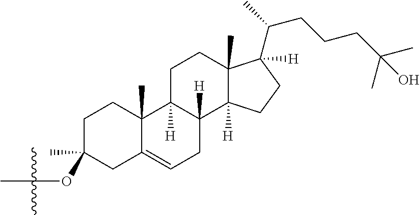 |
| 1126 | 1 | 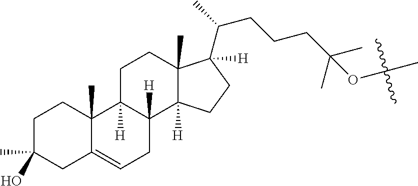 |
| 1127 | 2 | 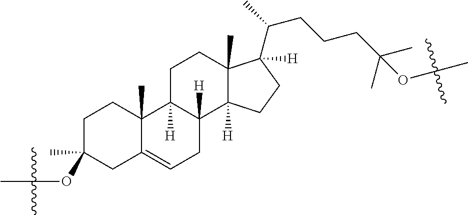 |
| 1128 | 1 | 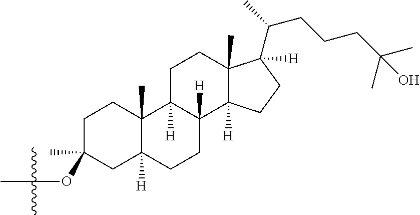 |
| 1129 | 1 | 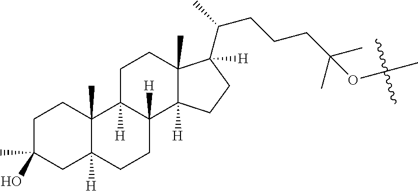 |
| 1130 | 2 | 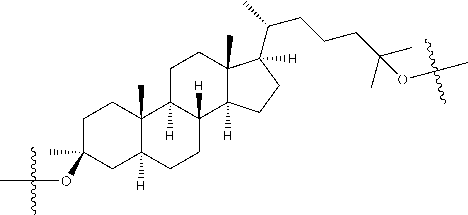 |
| 1131 | 1 | 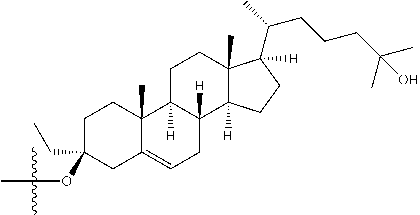 |
| 1132 | 1 | 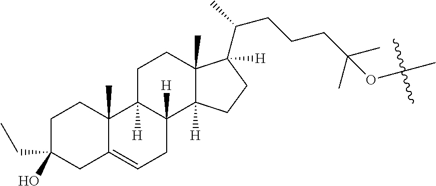 |
| 1133 | 2 | 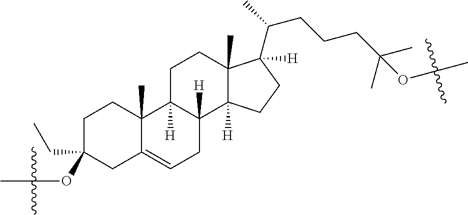 |
| 1134 | 1 | 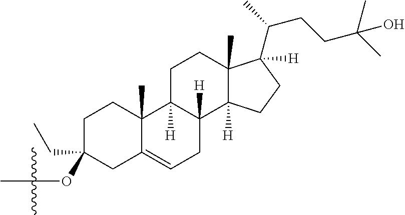 |
| 1135 | 1 | 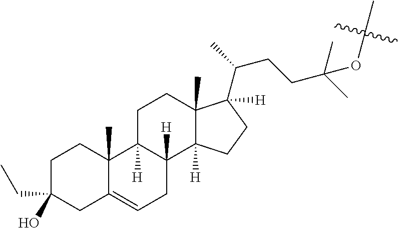 |
| 1136 | 2 | 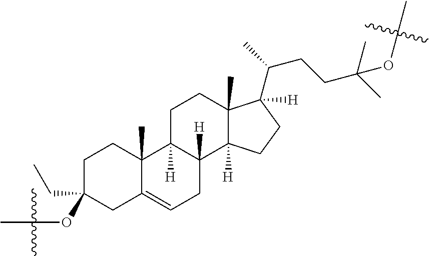 |

In some embodiments, the compound is selected from any of the compounds depicted in in Table 18, pharmaceutically acceptable salts thereof, deuterated variants thereof or combinations thereof.

TABLE 18

| | |
|---|---|
| | Formula (XLIV) |
| 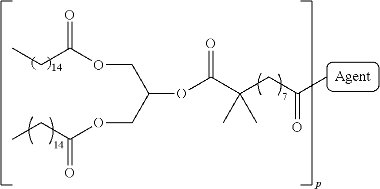 | |

| Cpd. No. | p | 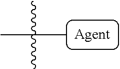 |
|---|---|---|
| 1137 | 1 | 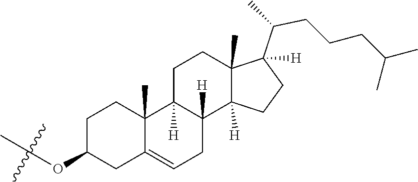 |
| 1138 | 1 | 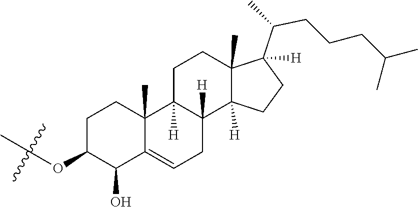 |
| 1139 | 1 | 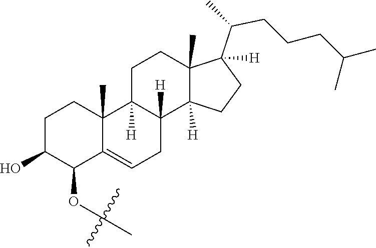 |
| 1140 | 2 | 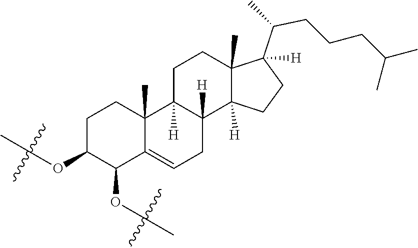 |
| 1141 | 1 | 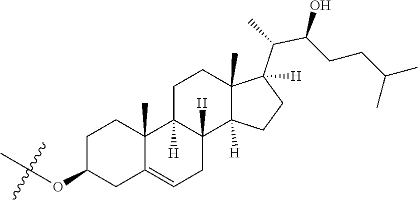 |
| 1142 | 1 | 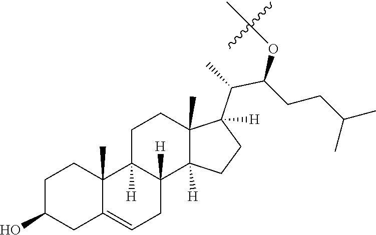 |
| 1143 | 2 | 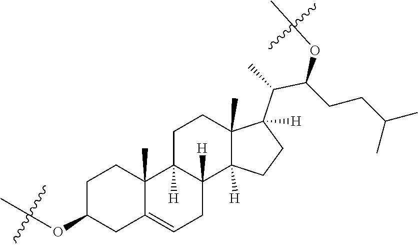 |
| 1144 | 1 | 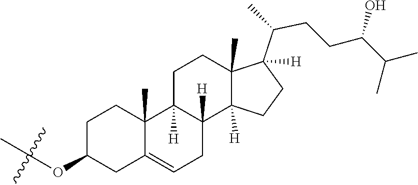 |
| 1145 | 1 | 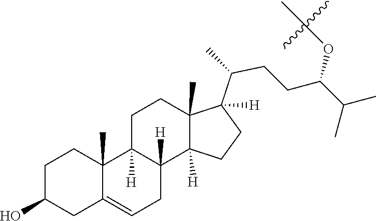 |
| 1146 | 2 | 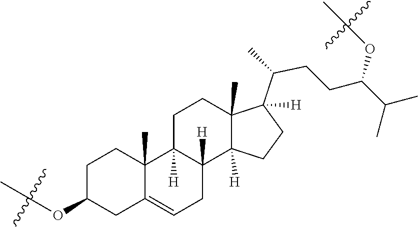 |
| 1147 | 1 | 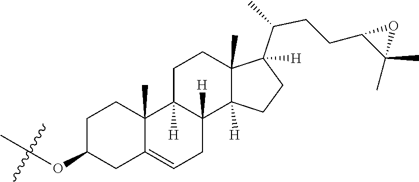 |
| 1148 | 1 | 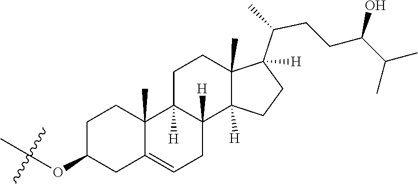 |
| 1149 | 1 | 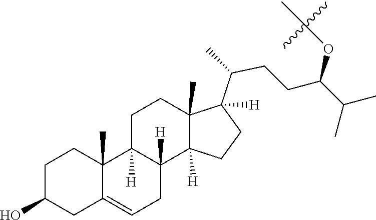 |
| 1150 | 2 | 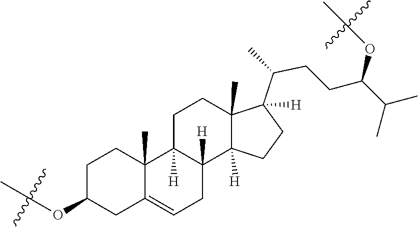 |
| 1151 | 1 | 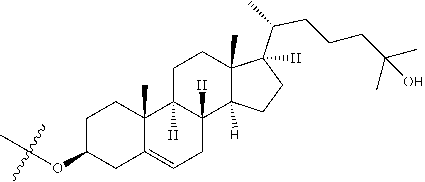 |
| 1152 | 1 | 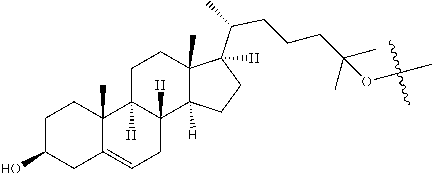 |
| 1153 | 2 | 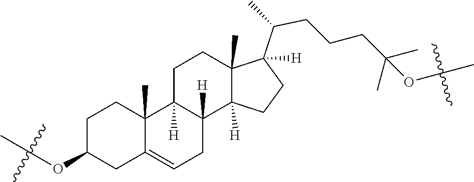 |
| 1154 | 1 | 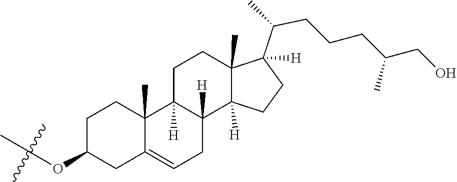 |
| 1155 | 1 | 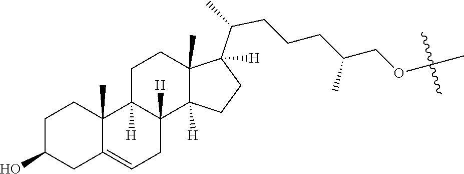 |
| 1156 | 2 | 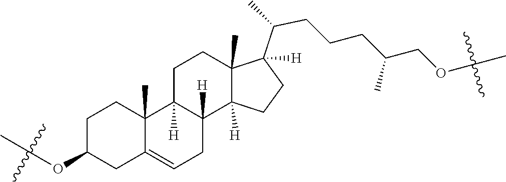 |
| 1157 | 1 | 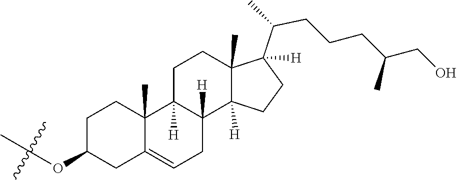 |
| 1158 | 1 | 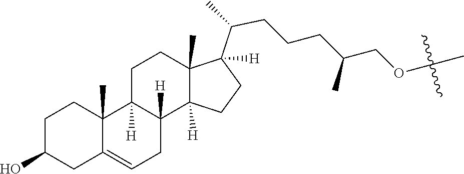 |
| 1159 | 2 | 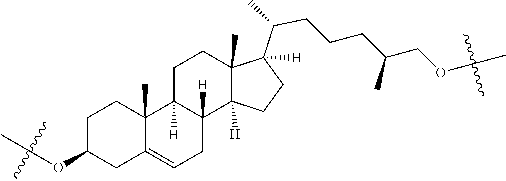 |
| 1160 | 1 | 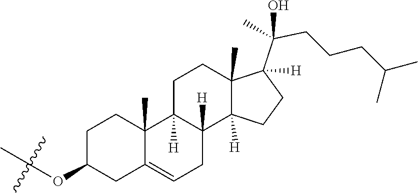 |
| 1161 | 1 | 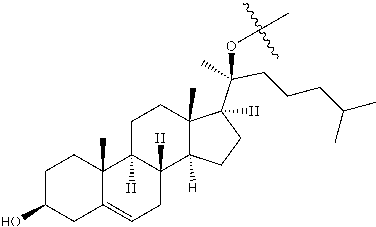 |
| 1162 | 2 | 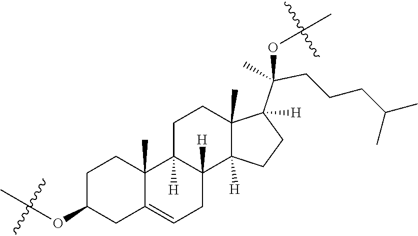 |
| 1163 | 1 | 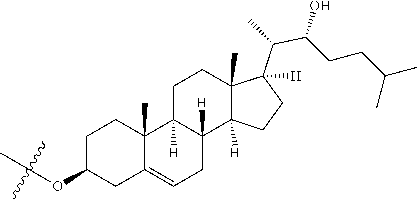 |
| 1164 | 1 | 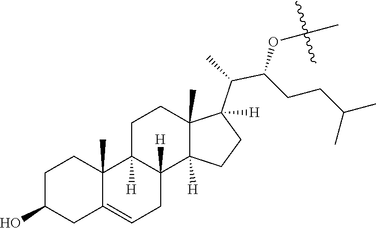 |
| 1165 | 2 | 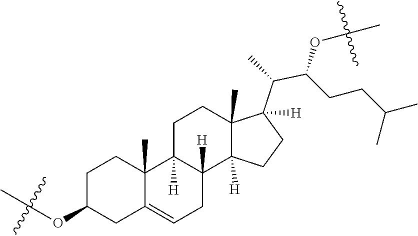 |
| 1166 | 1 | 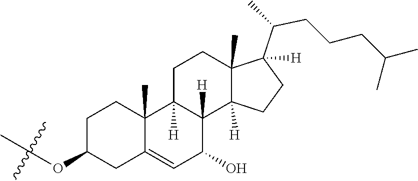 |
| 1167 | 1 | 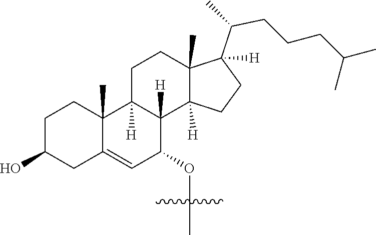 |
| 1168 | 2 | 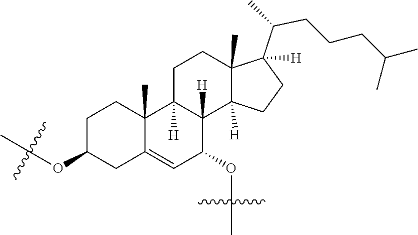 |
| 1169 | 1 | 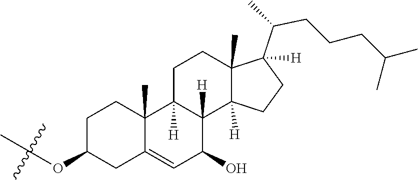 |
| 1170 | 1 | 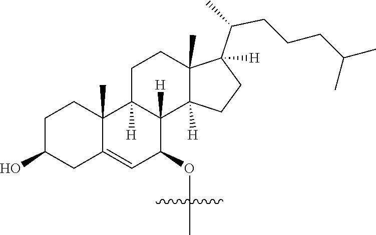 |
| 1171 | 2 | 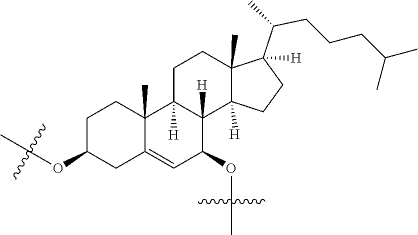 |
| 1172 | 1 | 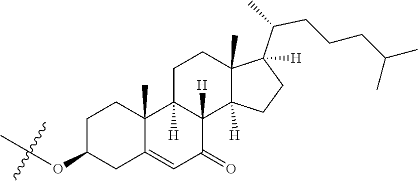 |
| 1173 | 1 | 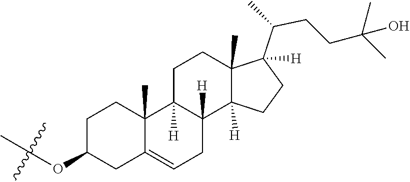 |
| 1174 | 1 | 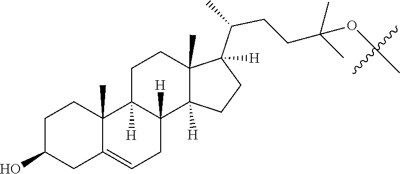 |
| 1175 | 2 | 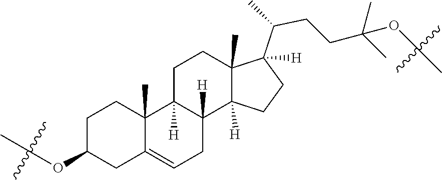 |
| 1176 | 1 | 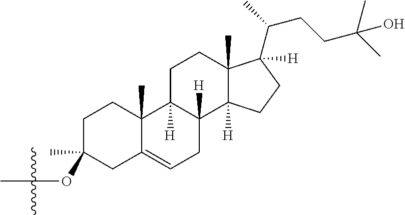 |
| 1177 | 1 | 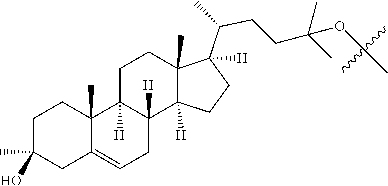 |
| 1178 | 2 | 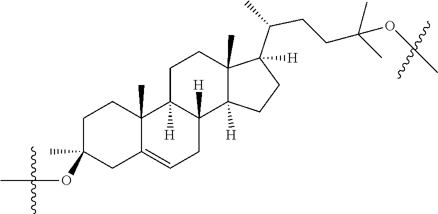 |
| 1179 | 1 | 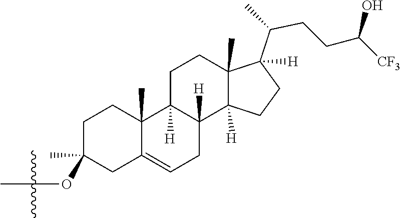 |
| 1180 | 1 | 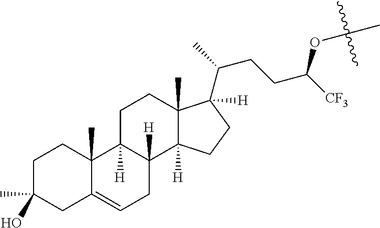 |
| 1181 | 2 | 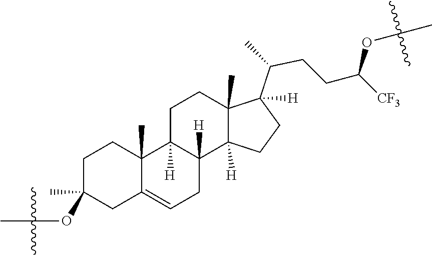 |
| 1182 | 1 | 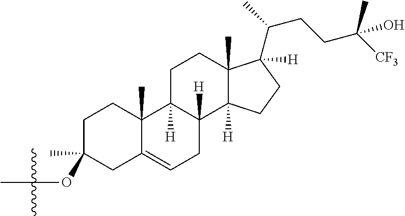 |
| 1183 | 1 | 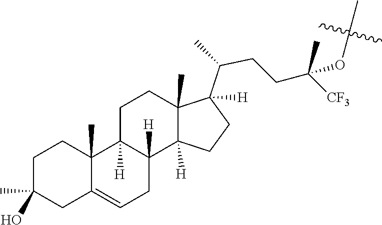 |
| 1184 | 2 | 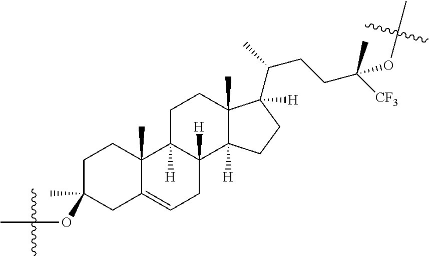 |
| 1185 | 1 | 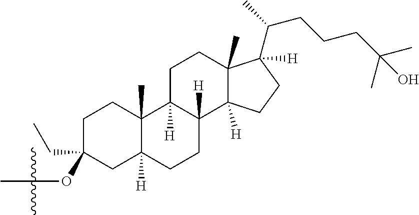 |
| 1186 | 1 | 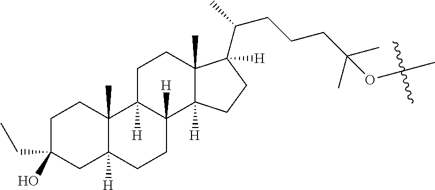 |
| 1187 | 2 | 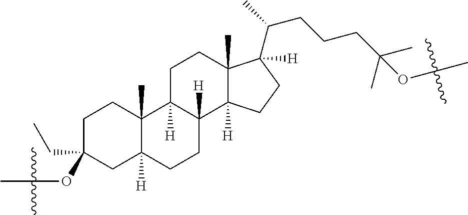 |
| 1188 | 1 | 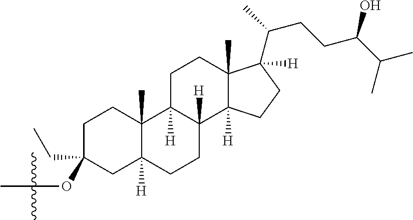 |
| 1189 | 1 | 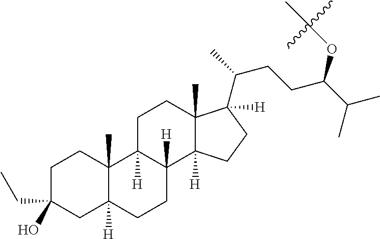 |
| 1190 | 2 | 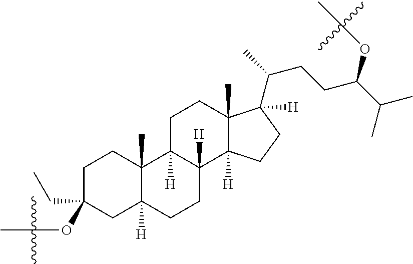 |
| 1191 | 1 | 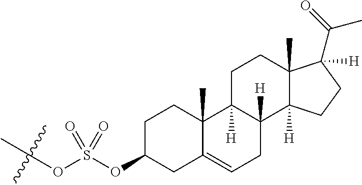 |
| 1192 | 1 | 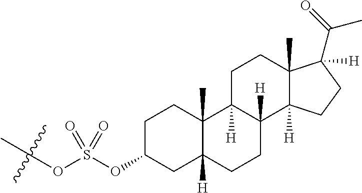 |
| 1193 | 1 | 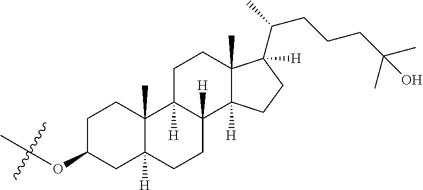 |
| 1194 | 1 | 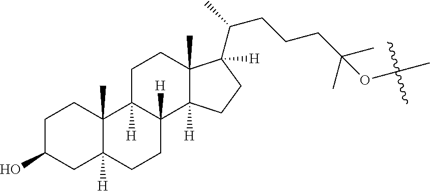 |
| 1195 | 2 | 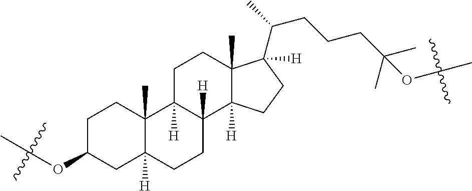 |
| 1196 | 1 | 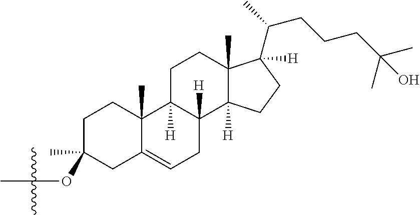 |
| 1197 | 1 | 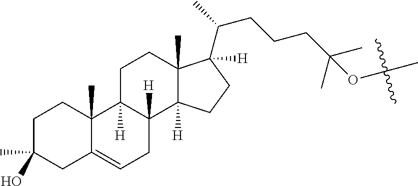 |
| 1198 | 2 | 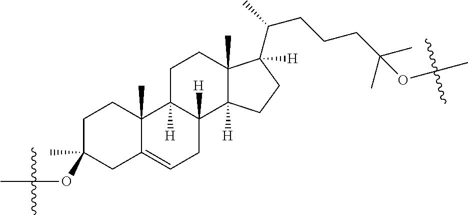 |
| 1199 | 1 | 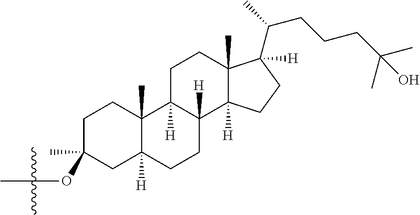 |
| 1200 | 1 | 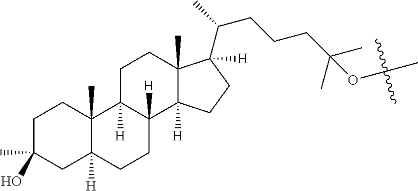 |
| 1201 | 2 | 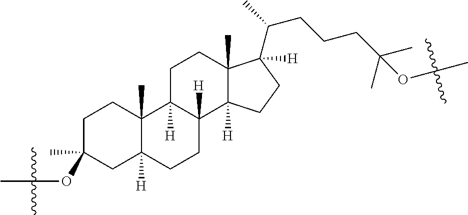 |
| 1202 | 1 | 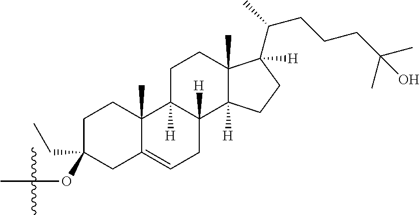 |
| 1203 | 1 | 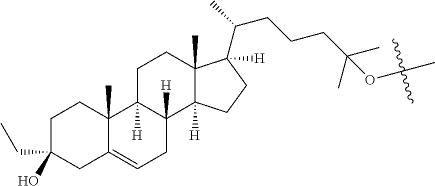 |
| 1204 | 2 | 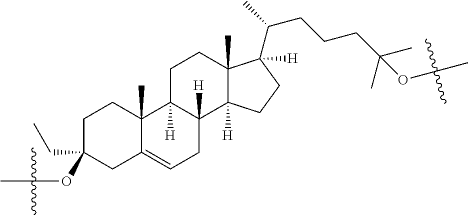 |
| 1205 | 1 | 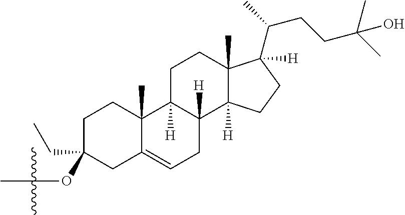 |
| 1206 | 1 | 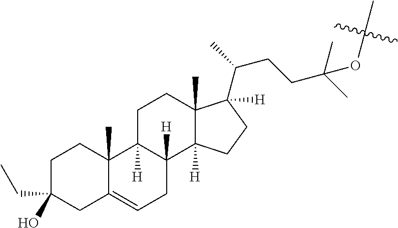 |
| 1207 | 2 | 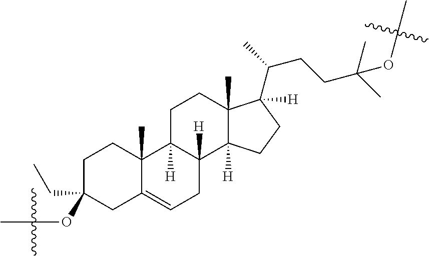 |

In some embodiments, the compound is selected from any of the compounds depicted in in Table 19, pharmaceutically acceptable salts thereof, deuterated variants thereof or combinations thereof.

TABLE 19

| | |
|---|---|
| | Formula (XLV) |
| 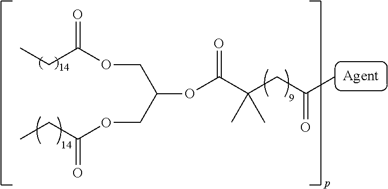 | |

| Cpd. No. | p | 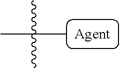 |
|---|---|---|
| 1208 | 1 | 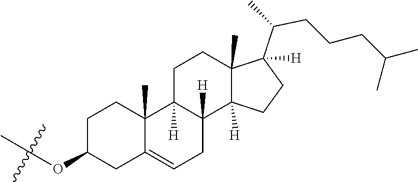 |
| 1209 | 1 | 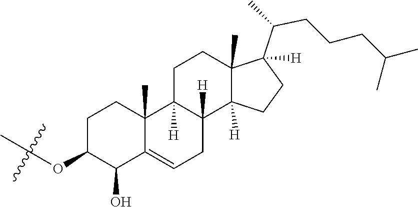 |
| 1210 | 1 | 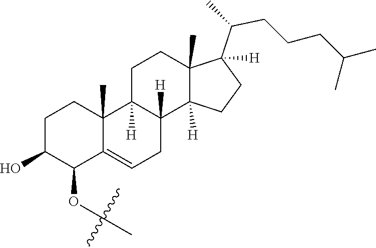 |
| 1211 | 2 | 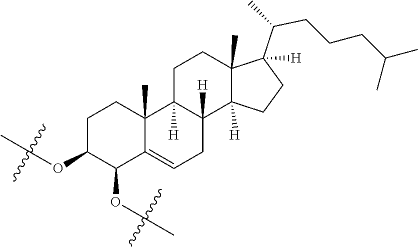 |
| 1212 | 1 | 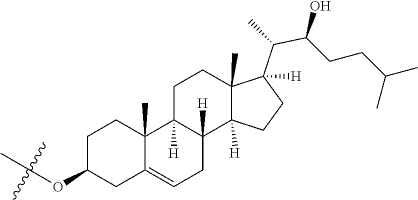 |
| 1213 | 1 | 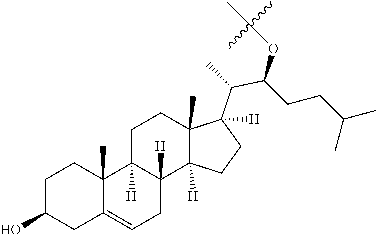 |
| 1214 | 2 | 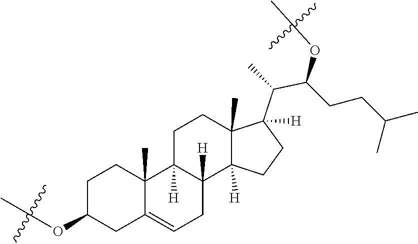 |
| 1215 | 1 | 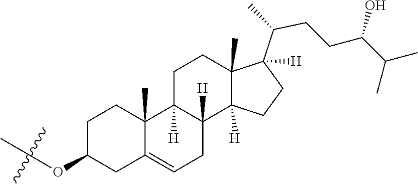 |
| 1216 | 1 | 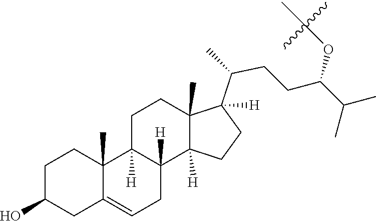 |
| 1217 | 2 | 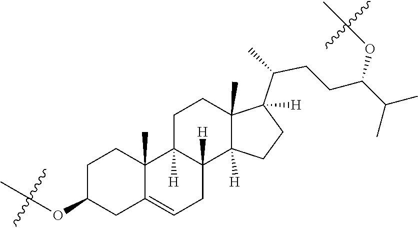 |
| 1218 | 1 | 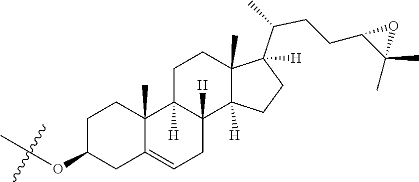 |
| 1219 | 1 | 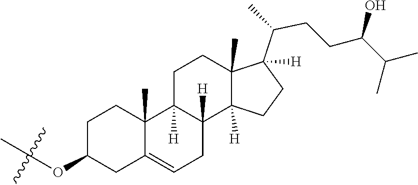 |
| 1220 | 1 | 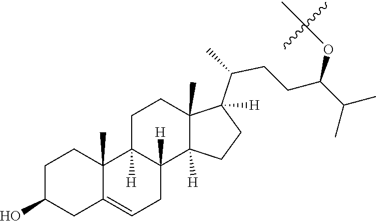 |
| 1221 | 2 | 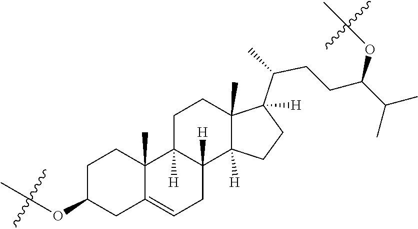 |
| 1222 | 1 | 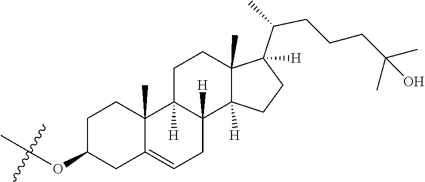 |
| 1223 | 1 | 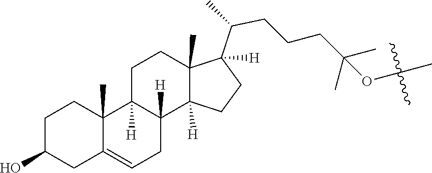 |
| 1224 | 2 | 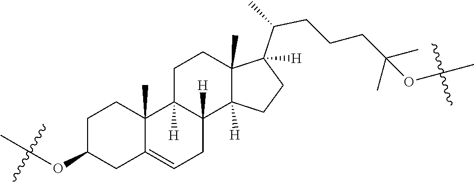 |
| 1225 | 1 | 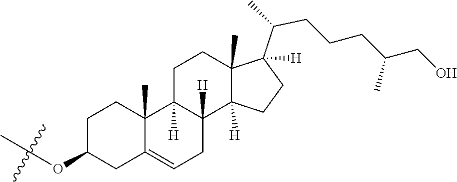 |
| 1226 | 1 | 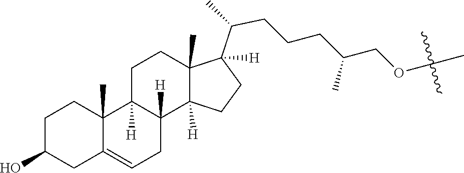 |
| 1227 | 2 | 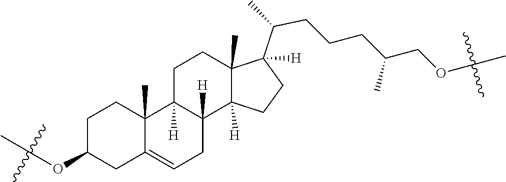 |
| 1228 | 1 | 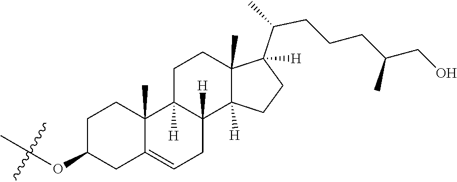 |
| 1229 | 1 | 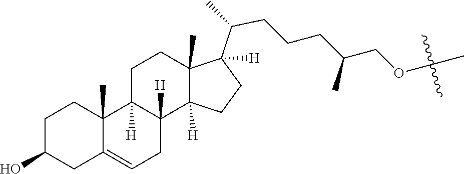 |
| 1230 | 2 | 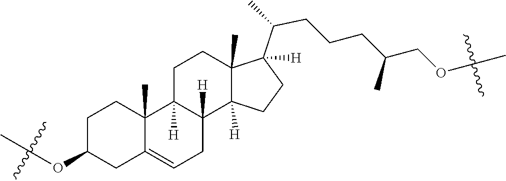 |
| 1231 | 1 | 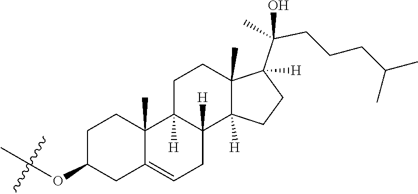 |
| 1232 | 1 | 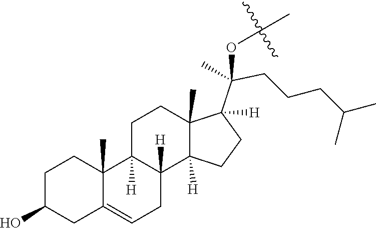 |
| 1233 | 2 | 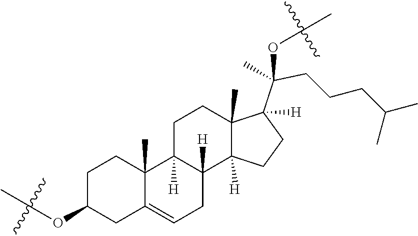 |
| 1234 | 1 | 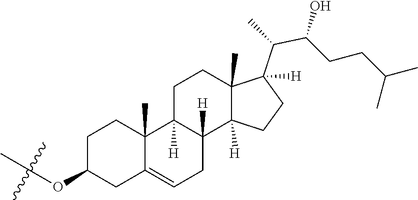 |
| 1235 | 1 | 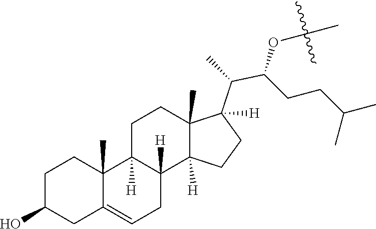 |
| 1236 | 2 | 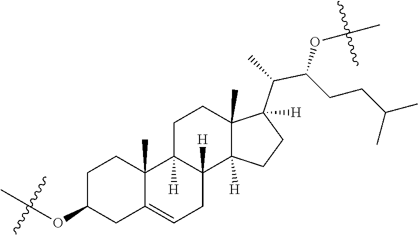 |
| 1237 | 1 | 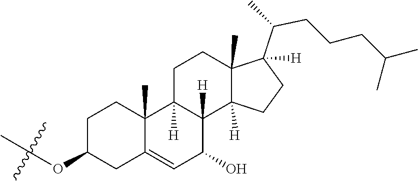 |
| 1238 | 1 | 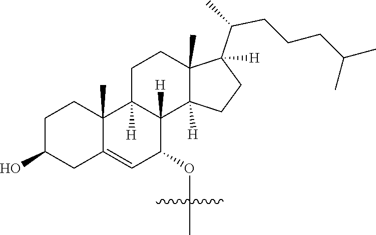 |
| 1239 | 2 | 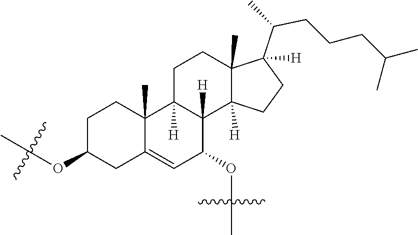 |
| 1240 | 1 | 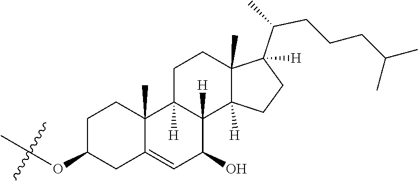 |
| 1241 | 1 | 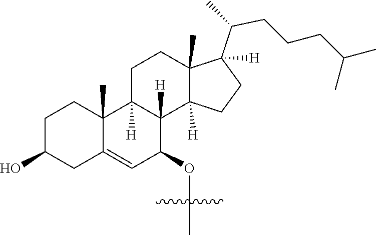 |
| 1242 | 2 | 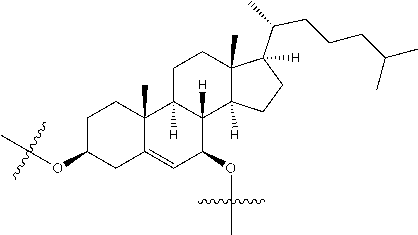 |
| 1243 | 1 | 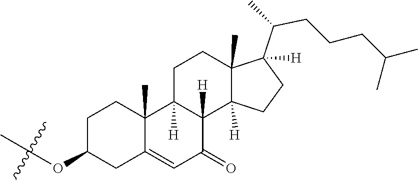 |
| 1244 | 1 | 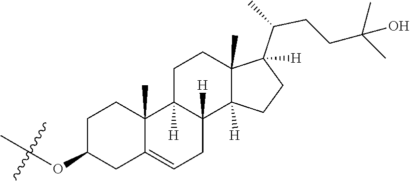 |
| 1245 | 1 | 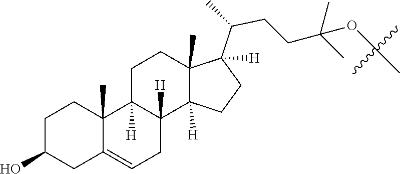 |
| 1246 | 2 | 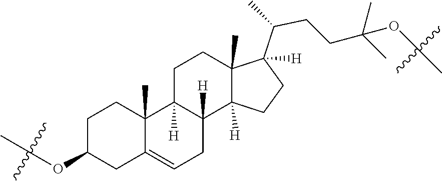 |
| 1247 | 1 | 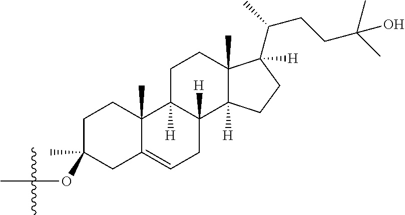 |
| 1248 | 1 | 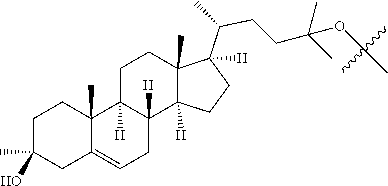 |
| 1249 | 2 | 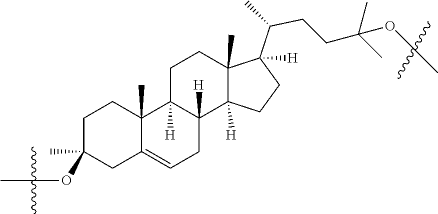 |
| 1250 | 1 | 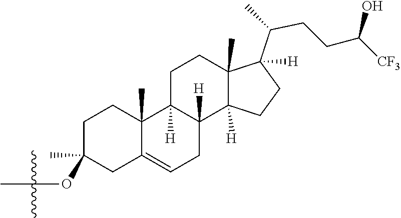 |
| 1251 | 1 | 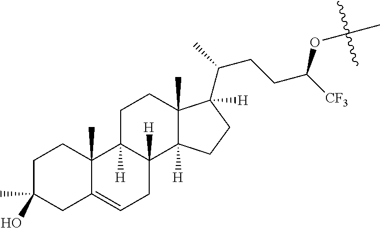 |
| 1252 | 2 | 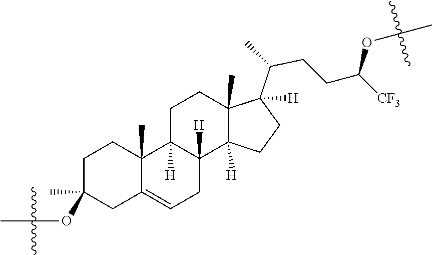 |
| 1253 | 1 | 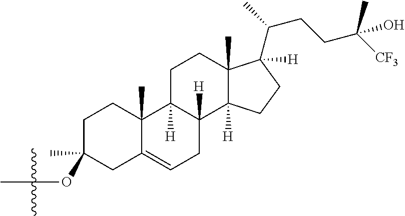 |
| 1254 | 1 | 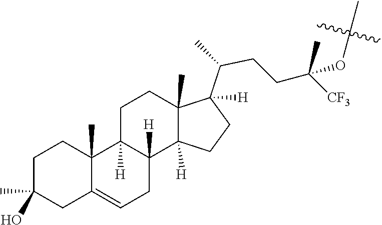 |
| 1255 | 2 | 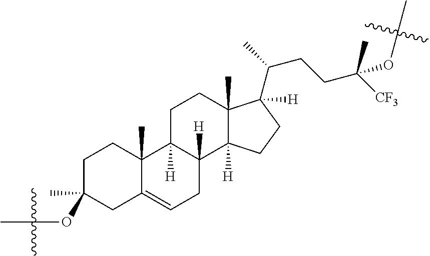 |
| 1256 | 1 | 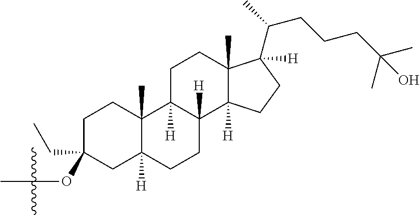 |
| 1257 | 1 | 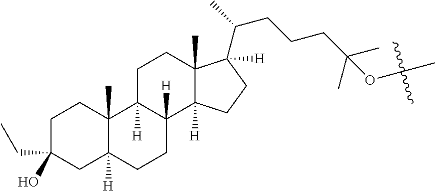 |
| 1258 | 2 | 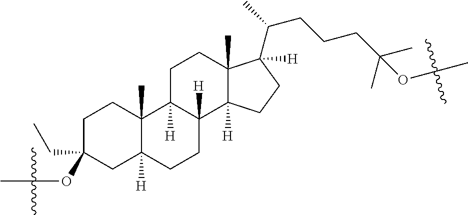 |
| 1259 | 1 | 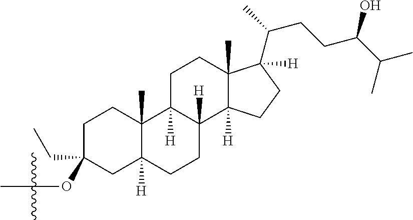 |
| 1260 | 1 | 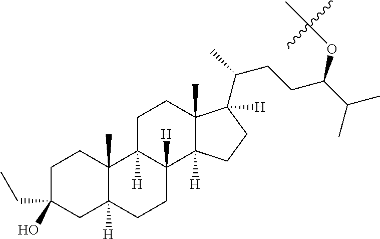 |
| 1261 | 2 | 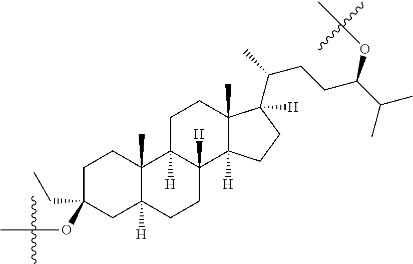 |
| 1262 | 1 | 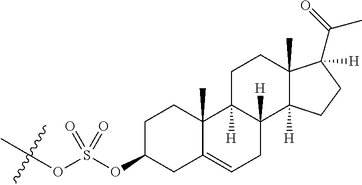 |
| 1263 | 1 | 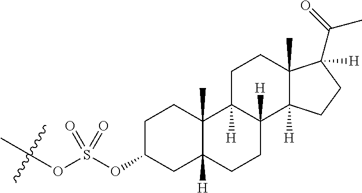 |
| 1264 | 1 | 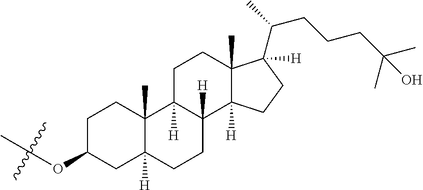 |
| 1265 | 1 | 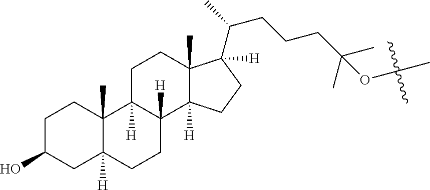 |
| 1266 | 2 | 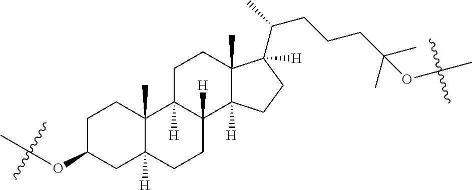 |
| 1267 | 1 | 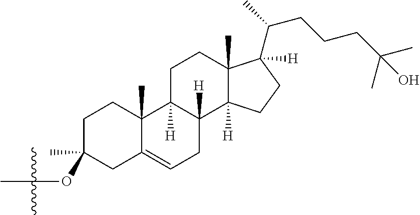 |
| 1268 | 1 | 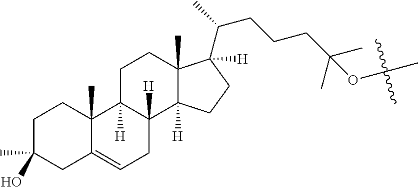 |
| 1269 | 2 | 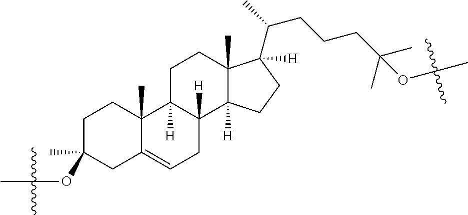 |
| 1270 | 1 | 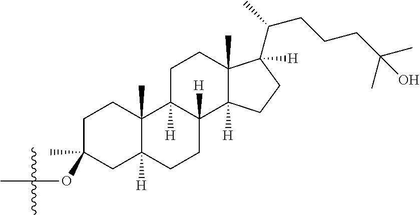 |
| 1271 | 1 | 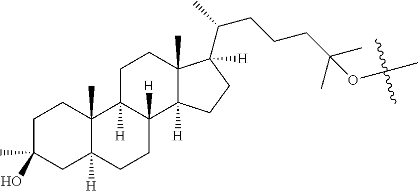 |
| 1272 | 2 | 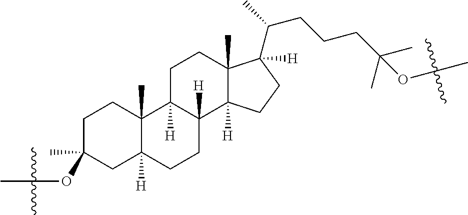 |
| 1273 | 1 | 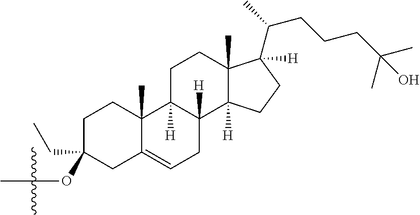 |
| 1274 | 1 | 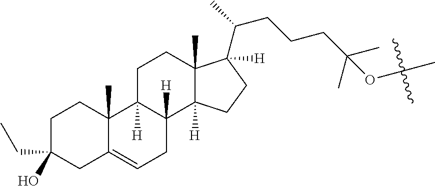 |
| 1275 | 2 | 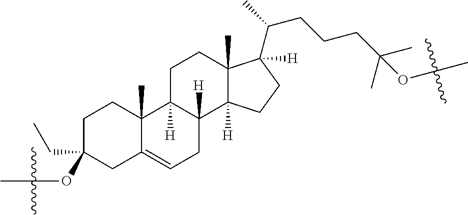 |
| 1276 | 1 | 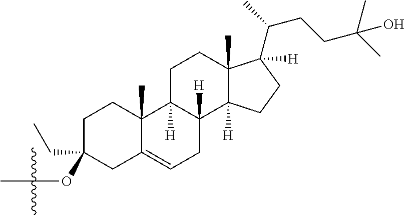 |
| 1277 | 1 | 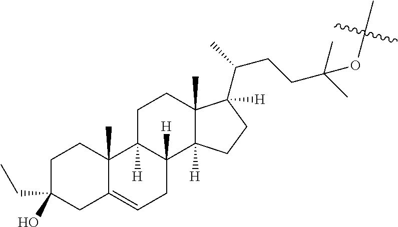 |
| 1278 | 2 | 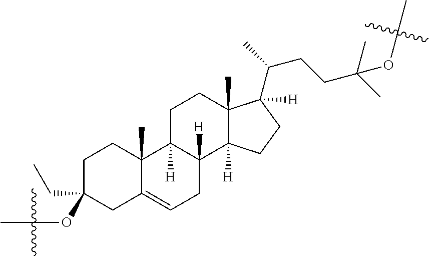 |

In some embodiments, the compound is selected from any of the compounds depicted in in Table 20, pharmaceutically acceptable salts thereof, deuterated variants thereof or combinations thereof.

TABLE 20
Formula (XLVI)

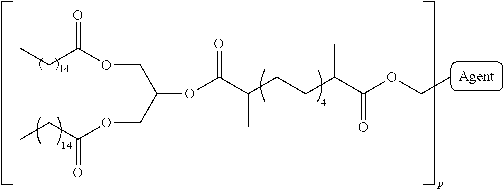

| Cpd. No. | p | 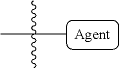 |
|---|---|---|
| 1279 | 1 | 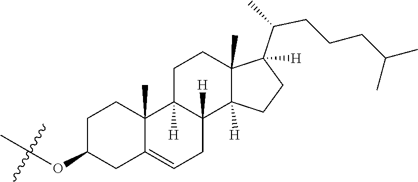 |
| 1280 | 1 | 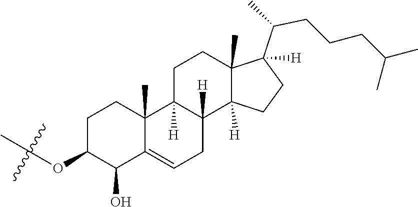 |
| 1281 | 1 | 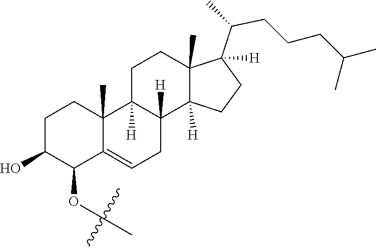 |
| 1282 | 2 | 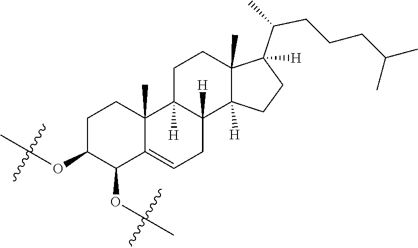 |
| 1283 | 1 | 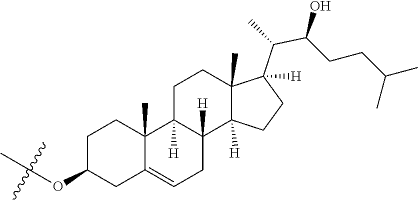 |
| 1284 | 1 | 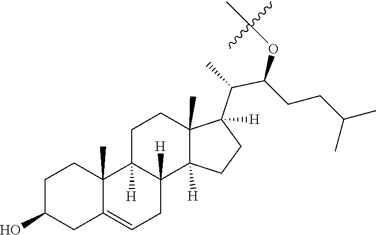 |
| 1285 | 2 | 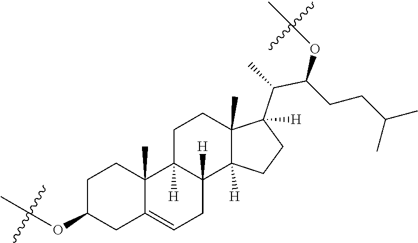 |
| 1286 | 1 | 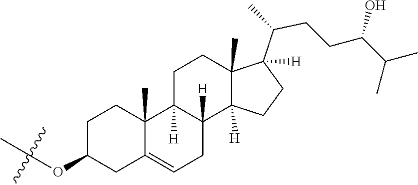 |
| 1287 | 1 | 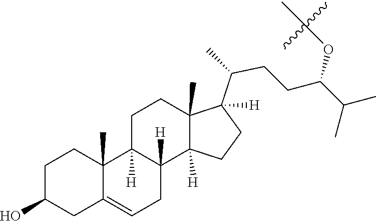 |
| 1288 | 2 | 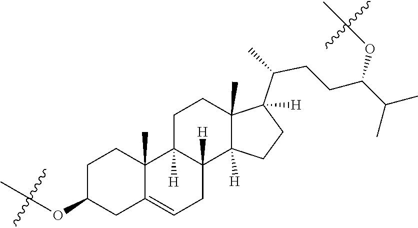 |
| 1289 | 1 | 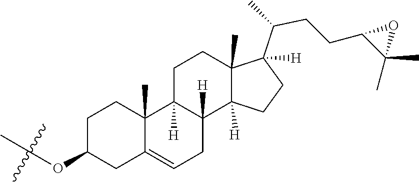 |
| 1290 | 1 | 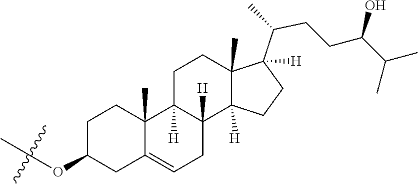 |
| 1291 | 1 | 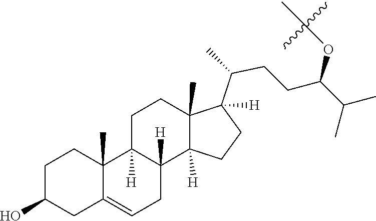 |
| 1292 | 2 | 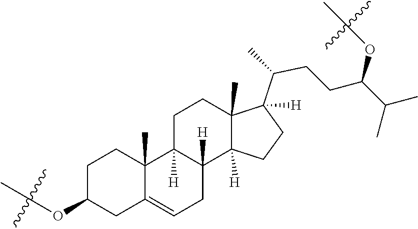 |
| 1293 | 1 | 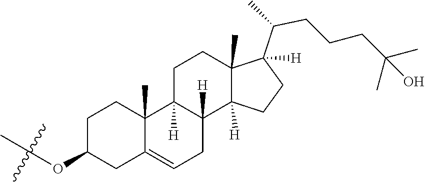 |
| 1294 | 1 | 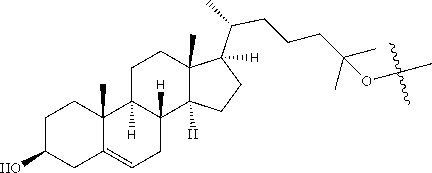 |
| 1295 | 2 | 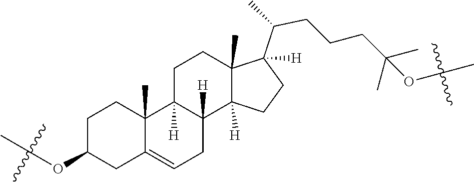 |
| 1296 | 1 | 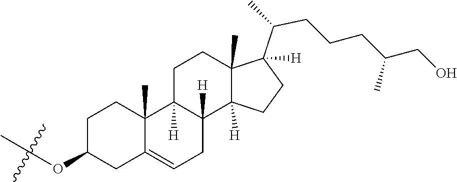 |
| 1297 | 1 | 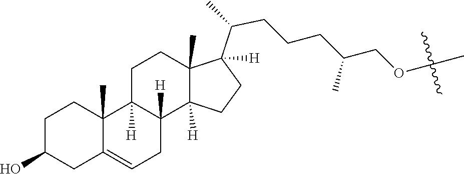 |
| 1298 | 2 | 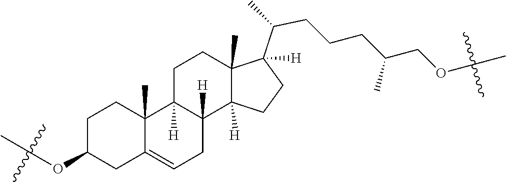 |
| 1299 | 1 | 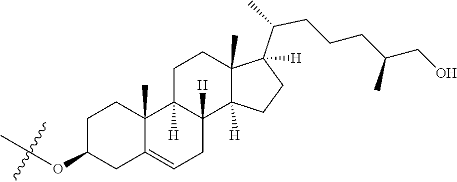 |
| 1300 | 1 | 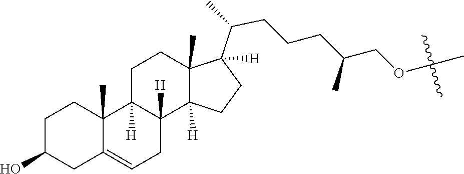 |
| 1301 | 2 | 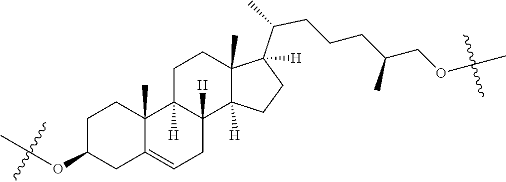 |
| 1302 | 1 | 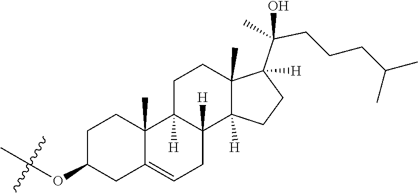 |
| 1303 | 1 | 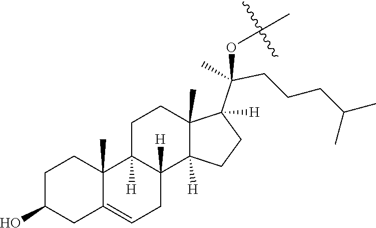 |
| 1304 | 2 | 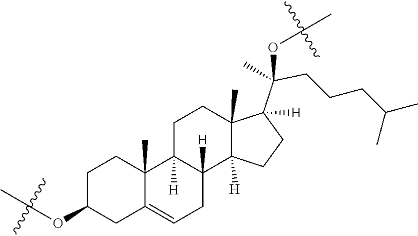 |
| 1305 | 1 | 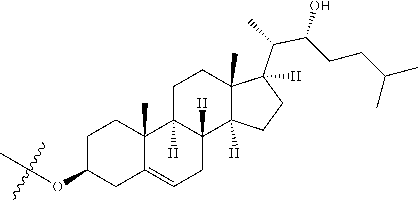 |
| 1306 | 1 | 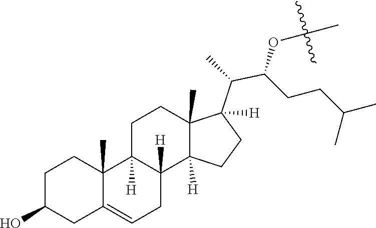 |
| 1307 | 2 | 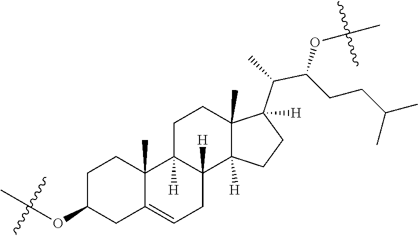 |
| 1308 | 1 | 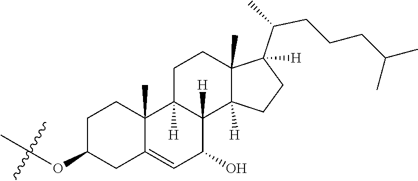 |
| 1309 | 1 | 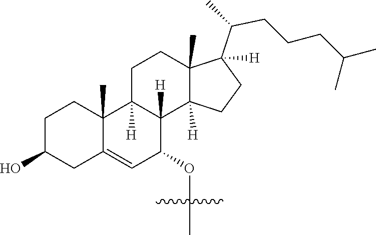 |
| 1310 | 2 | 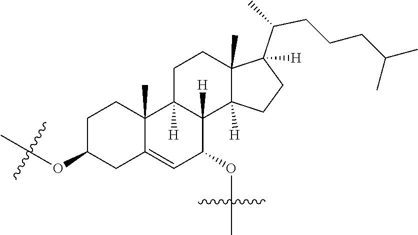 |
| 1311 | 1 | 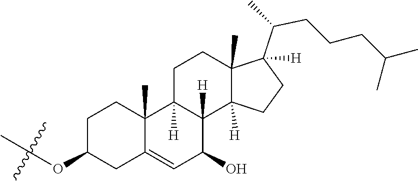 |
| 1312 | 1 | 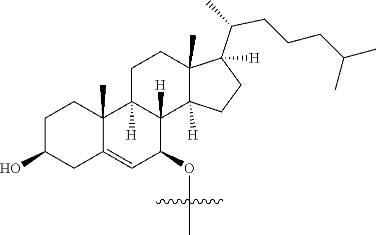 |
| 1313 | 2 | 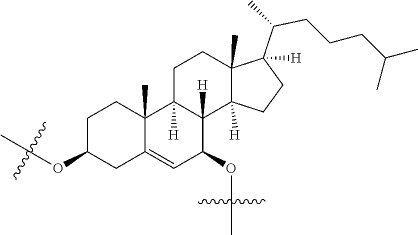 |
| 1314 | 1 | 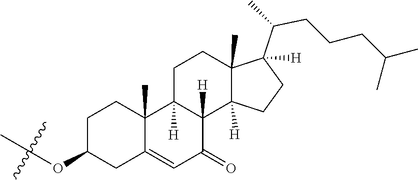 |
| 1315 | 1 | 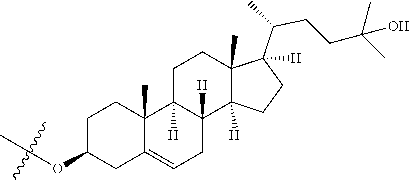 |
| 1316 | 1 | 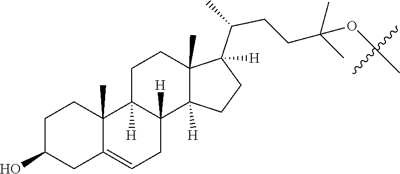 |
| 1317 | 2 | 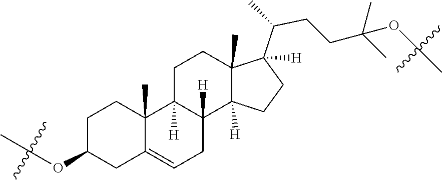 |
| 1318 | 1 | 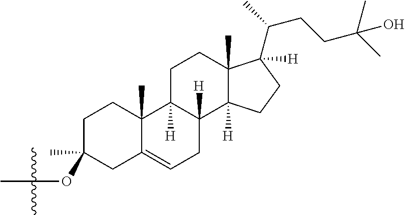 |
| 1319 | 1 | 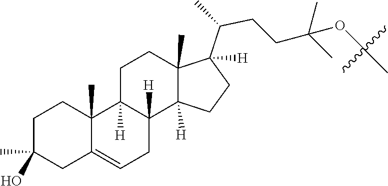 |
| 1320 | 2 | 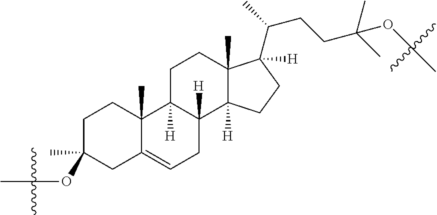 |
| 1321 | 1 | 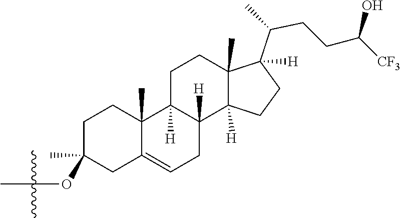 |
| 1322 | 1 | 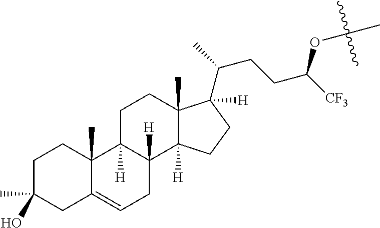 |
| 1323 | 2 | 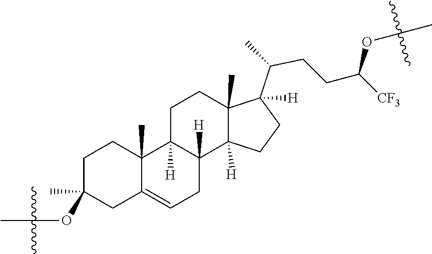 |
| 1324 | 1 | 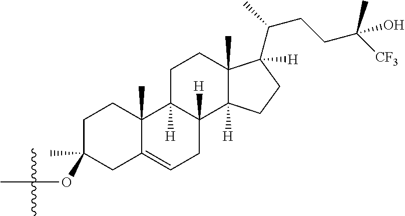 |
| 1325 | 1 | 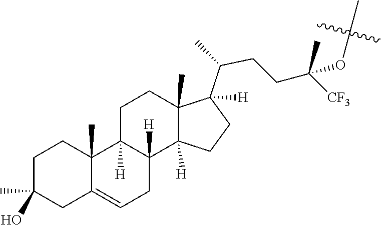 |
| 1326 | 2 | 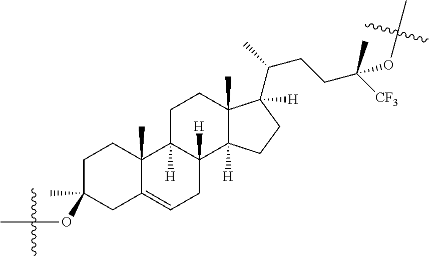 |
| 1327 | 1 | 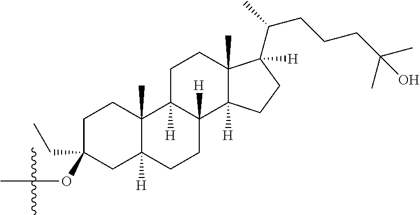 |
| 1328 | 1 | 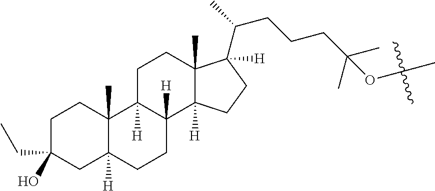 |
| 1329 | 2 | 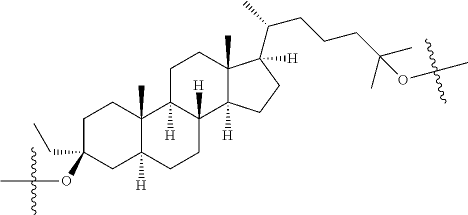 |
| 1330 | 1 | 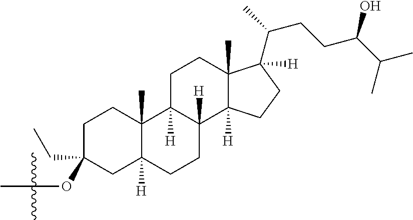 |
| 1331 | 1 | 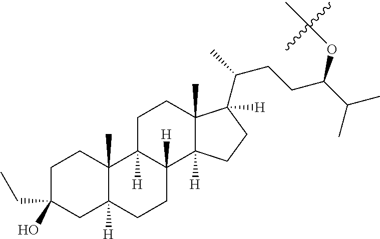 |
| 1332 | 2 | 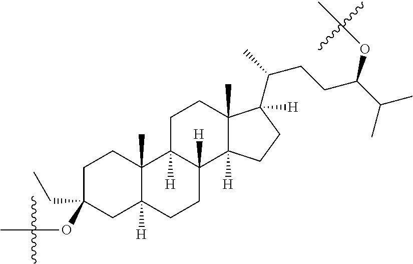 |
| 1333 | 1 | 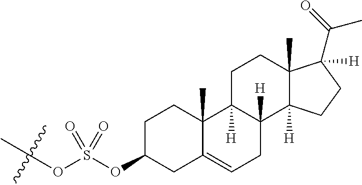 |
| 1334 | 1 | 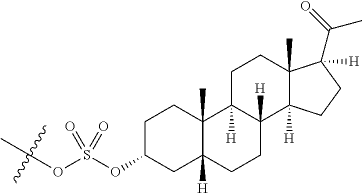 |
| 1335 | 1 | 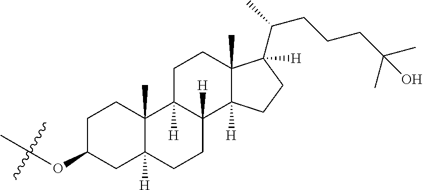 |
| 1336 | 1 | 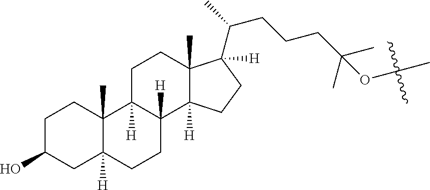 |
| 1337 | 2 | 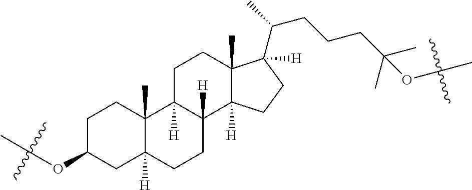 |
| 1338 | 1 | 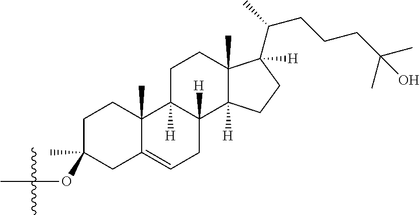 |
| 1339 | 1 | 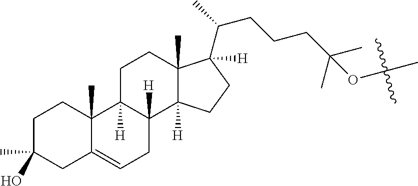 |
| 1340 | 2 | 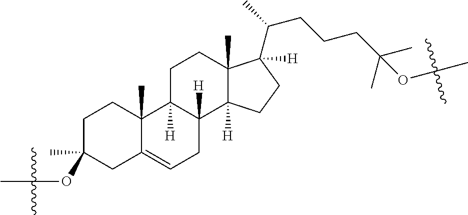 |
| 1341 | 1 | 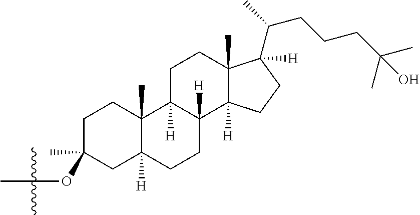 |
| 1342 | 1 | 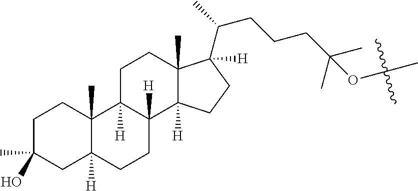 |
| 1343 | 2 | 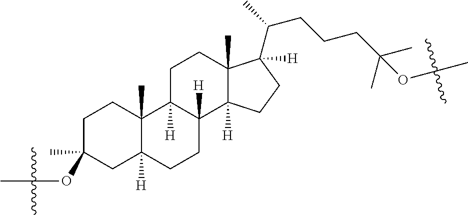 |
| 1344 | 1 | 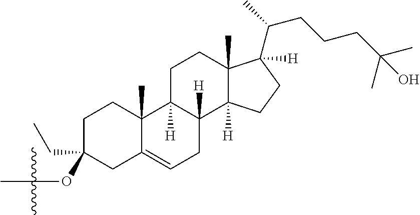 |
| 1345 | 1 | 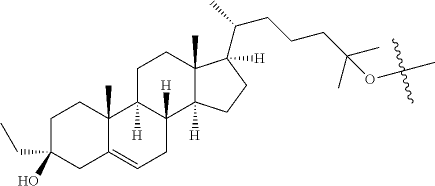 |
| 1346 | 2 | 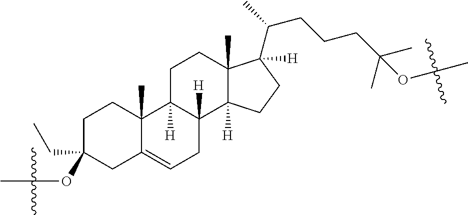 |
| 1347 | 1 | 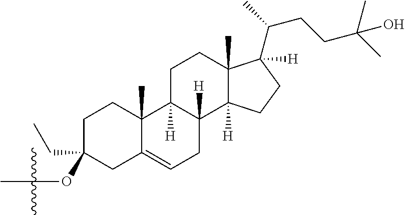 |
| 1348 | 1 | 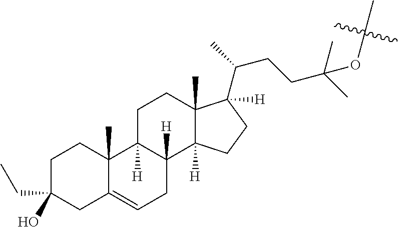 |
| 1349 | 2 | 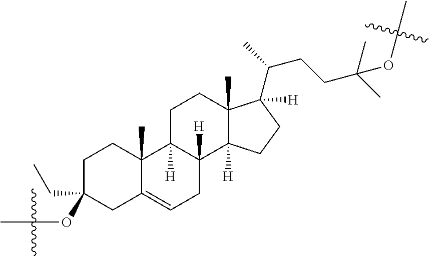 |

In some embodiments, the compound is selected from any of the compounds depicted in in Table 21, pharmaceutically acceptable salts thereof, deuterated variants thereof or combinations thereof.

TABLE 21
Formula (XLVII)

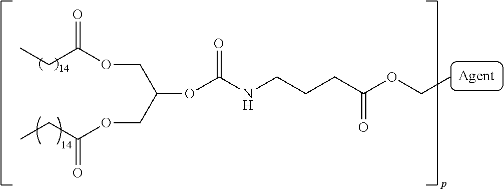

| Cpd. No. | p | 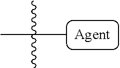 |
|---|---|---|
| 1350 | 1 | 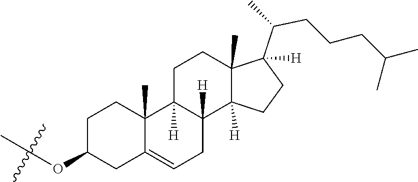 |
| 1351 | 1 | 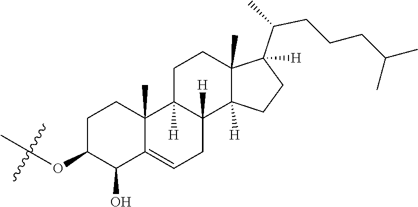 |
| 1352 | 1 | 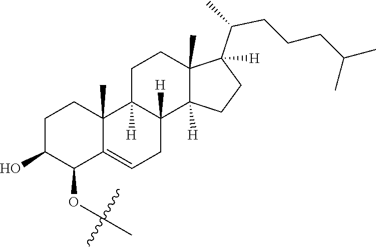 |
| 1353 | 2 | 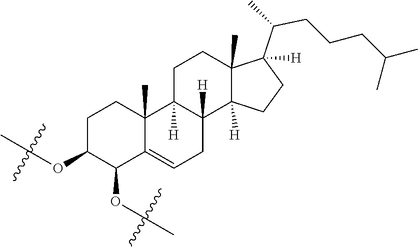 |
| 1354 | 1 | 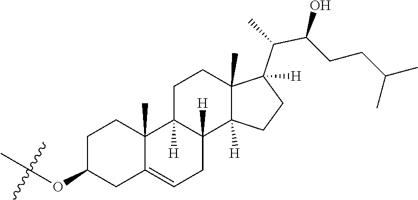 |
| 1355 | 1 | 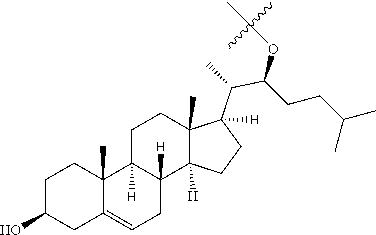 |
| 1356 | 2 | 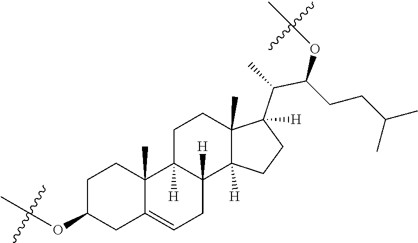 |
| 1357 | 1 | 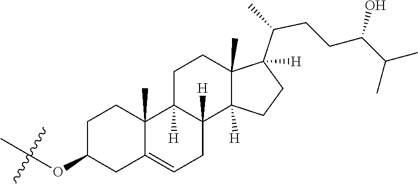 |
| 1358 | 1 | 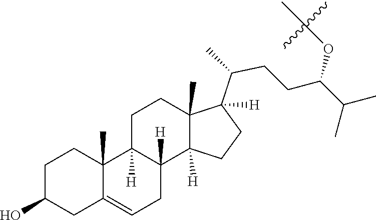 |
| 1359 | 2 | 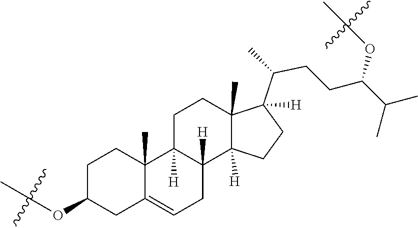 |
| 1360 | 1 | 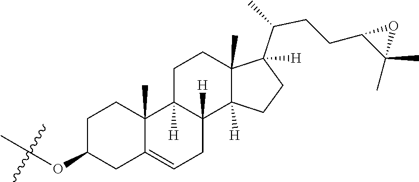 |
| 1361 | 1 | 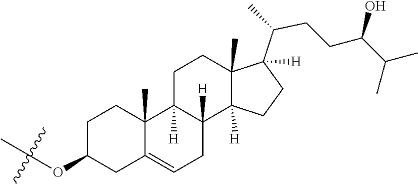 |
| 1362 | 1 | 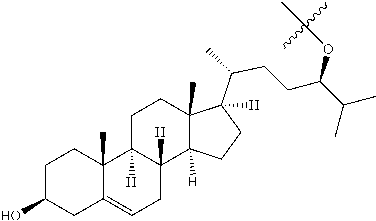 |
| 1363 | 2 | 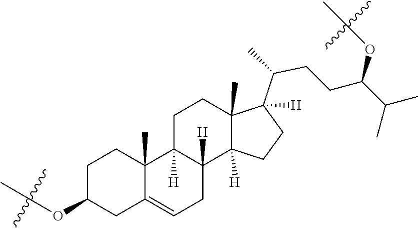 |
| 1364 | 1 | 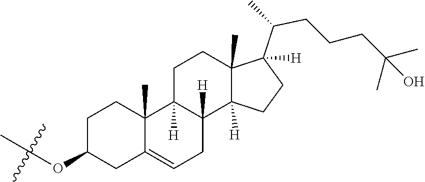 |
| 1365 | 1 | 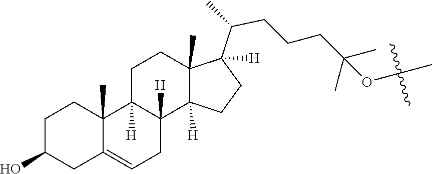 |
| 1366 | 2 | 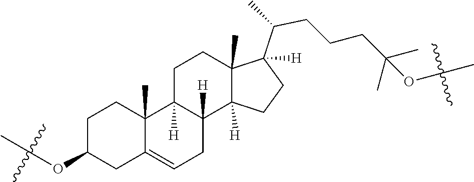 |
| 1367 | 1 | 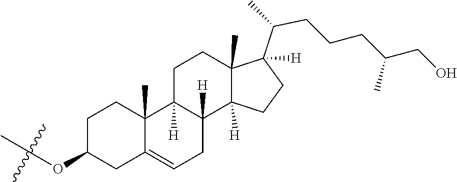 |
| 1368 | 1 | 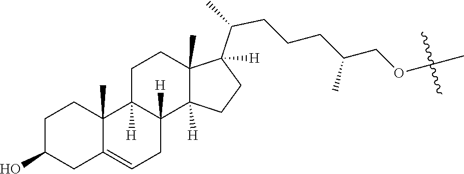 |
| 1369 | 2 | 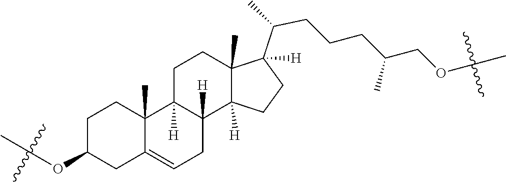 |
| 1370 | 1 | 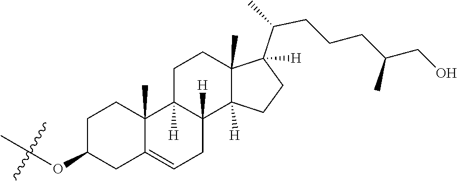 |
| 1371 | 1 | 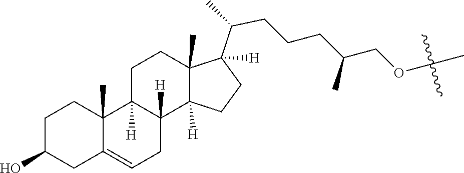 |
| 1372 | 2 | 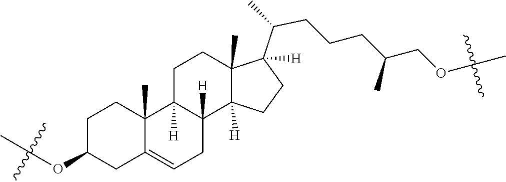 |
| 1373 | 1 | 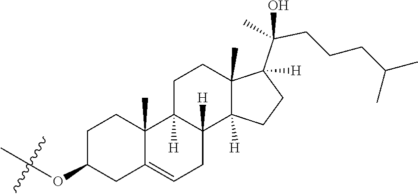 |
| 1374 | 1 | 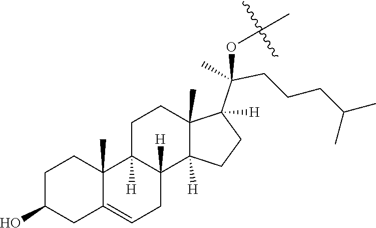 |
| 1375 | 2 | 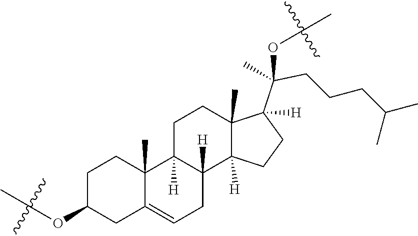 |
| 1376 | 1 | 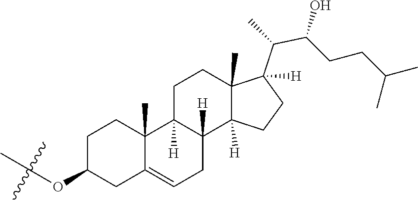 |
| 1377 | 1 | 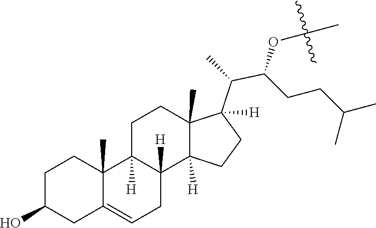 |
| 1378 | 2 | 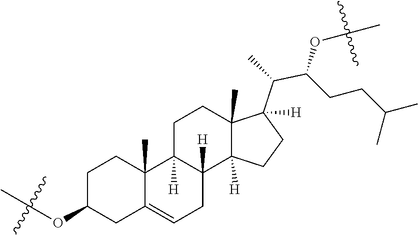 |
| 1379 | 1 | 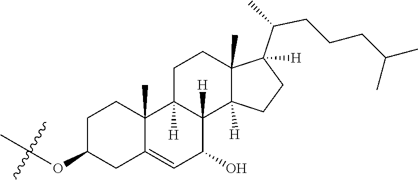 |
| 1380 | 1 | 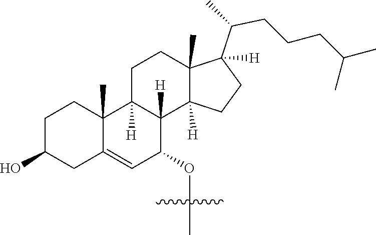 |
| 1381 | 2 | 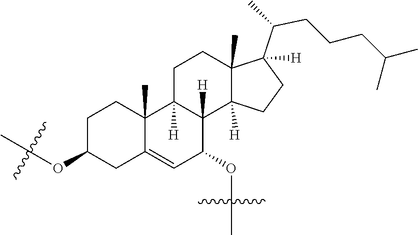 |
| 1382 | 1 | 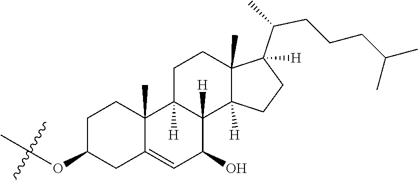 |
| 1383 | 1 | 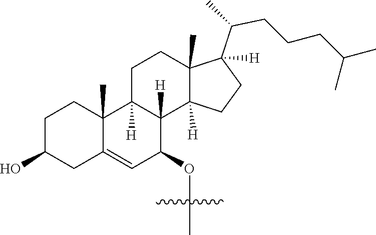 |
| 1384 | 2 | 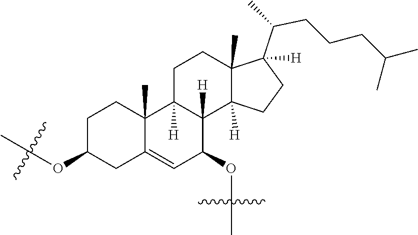 |
| 1385 | 1 | 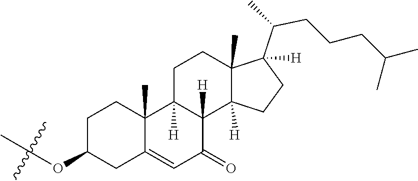 |
| 1386 | 1 | 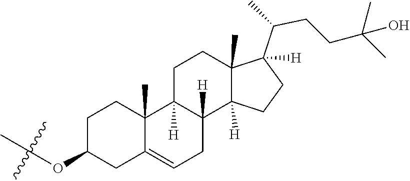 |
| 1387 | 1 | 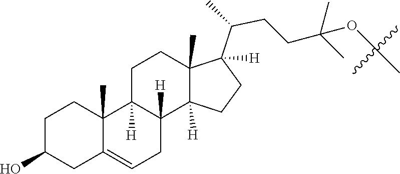 |
| 1388 | 2 | 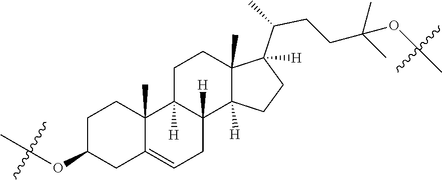 |
| 1389 | 1 | 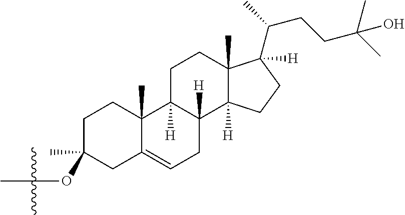 |
| 1390 | 1 | 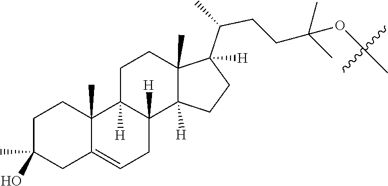 |
| 1391 | 2 | 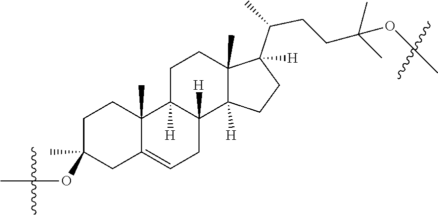 |
| 1392 | 1 | 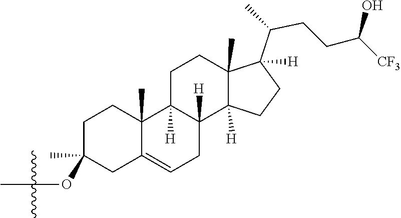 |
| 1393 | 1 | 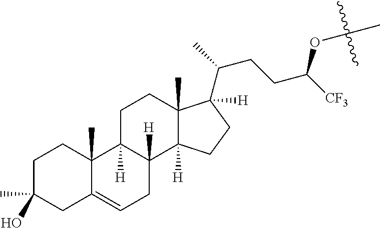 |
| 1394 | 2 | 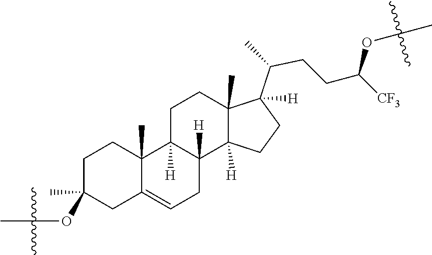 |
| 1395 | 1 | 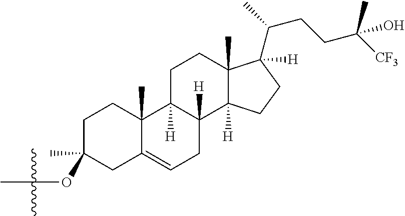 |
| 1396 | 1 | 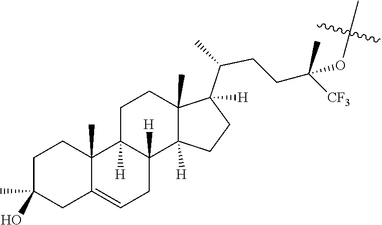 |
| 1397 | 2 | 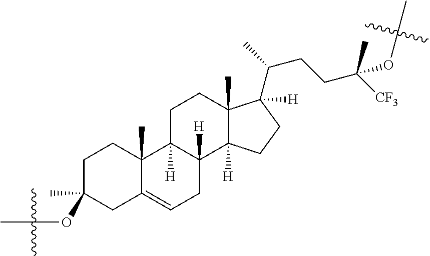 |
| 1398 | 1 | 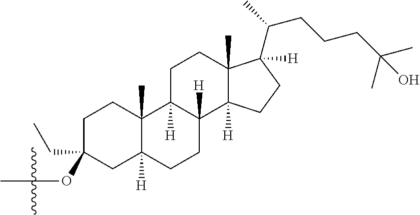 |
| 1399 | 1 | 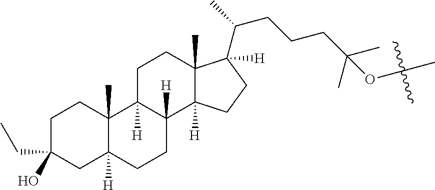 |
| 1400 | 2 | 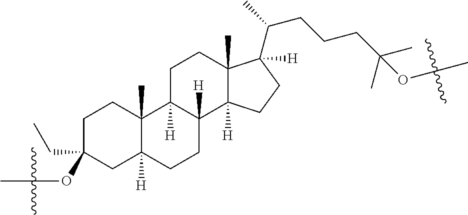 |
| 1401 | 1 | 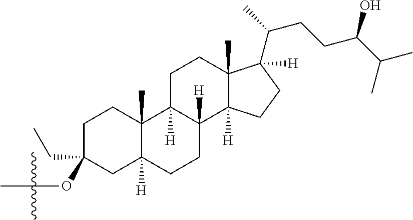 |
| 1402 | 1 | 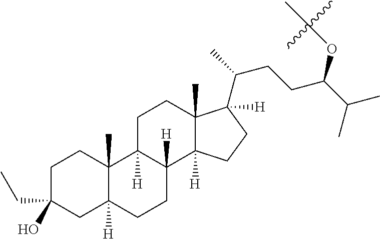 |
| 1403 | 2 | 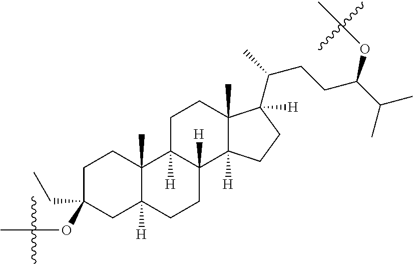 |
| 1404 | 1 | 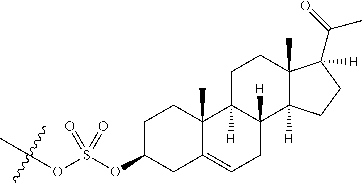 |
| 1405 | 1 | 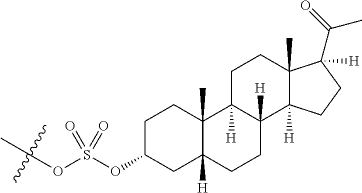 |
| 1406 | 1 | 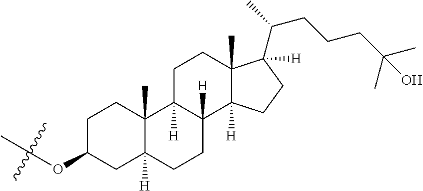 |
| 1407 | 1 | 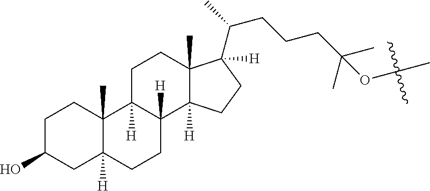 |
| 1408 | 2 | 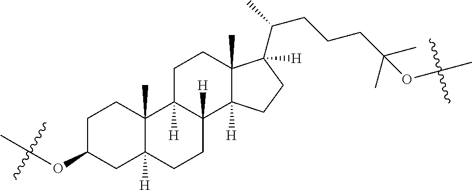 |
| 1409 | 1 | 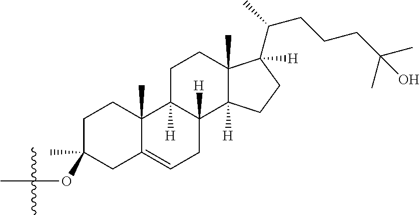 |
| 1410 | 1 | 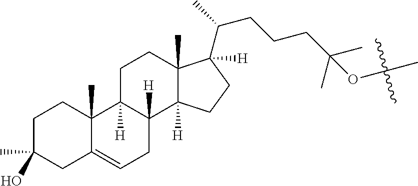 |
| 1411 | 2 | 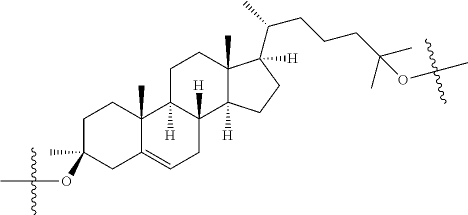 |
| 1412 | 1 | 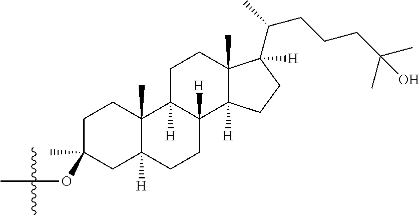 |
| 1413 | 1 | 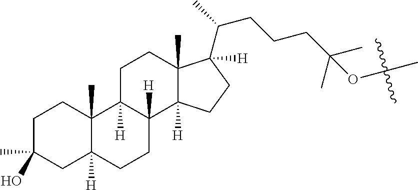 |
| 1414 | 2 | 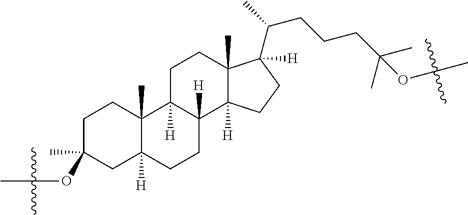 |
| 1415 | 1 | 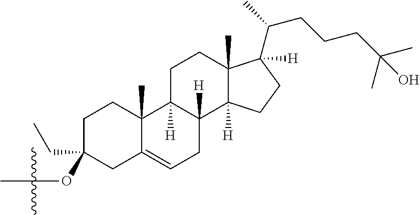 |
| 1416 | 1 | 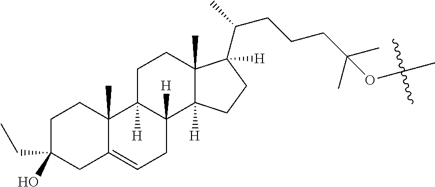 |
| 1417 | 2 | 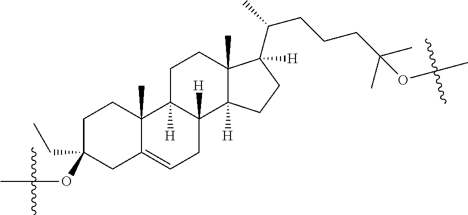 |
| 1418 | 1 | 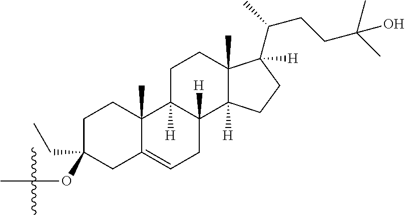 |
| 1419 | 1 | 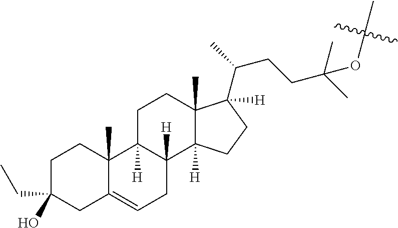 |
| 1420 | 2 | 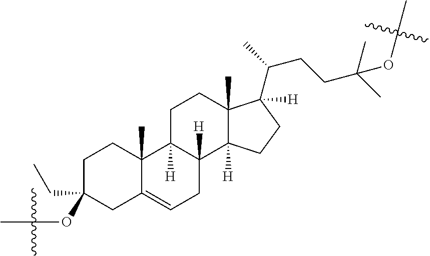 |

In some embodiments, the compound is selected from any of the compounds depicted in in Table 22, pharmaceutically acceptable salts thereof, deuterated variants thereof or combinations thereof.

TABLE 22
Formula (XLVIII)

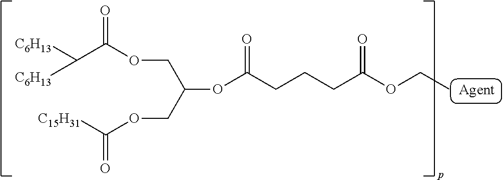

| Cpd. No. | p | 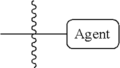 |
|---|---|---|
| 1421 | 1 | 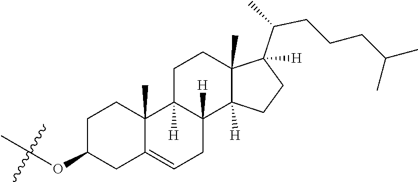 |
| 1422 | 1 | 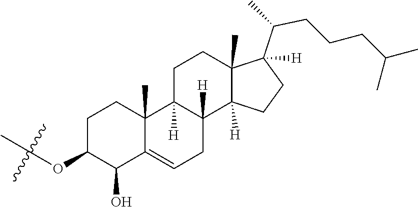 |
| 1423 | 1 | 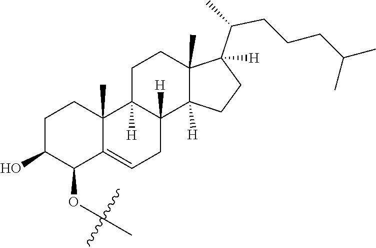 |
| 1424 | 2 | 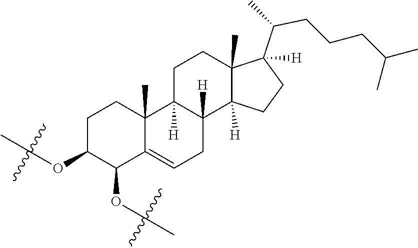 |
| 1425 | 1 | 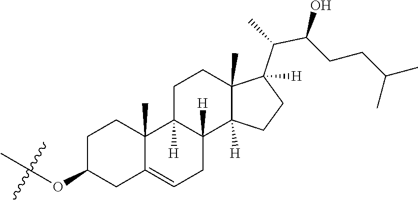 |
| 1426 | 1 | 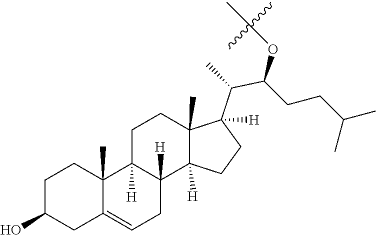 |
| 1427 | 2 | 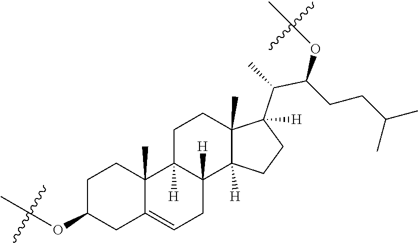 |
| 1428 | 1 | 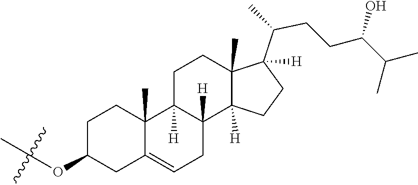 |
| 1429 | 1 | 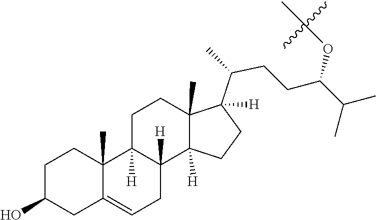 |
| 1430 | 2 | 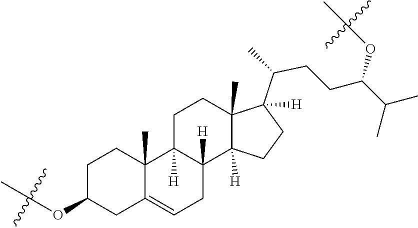 |
| 1431 | 1 | 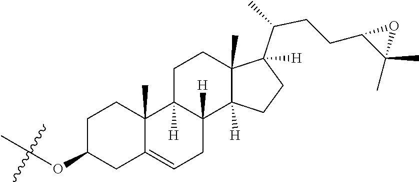 |
| 1432 | 1 | 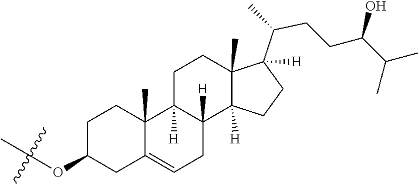 |
| 1433 | 1 | 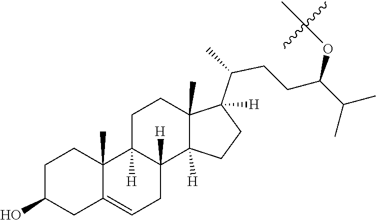 |
| 1434 | 2 | 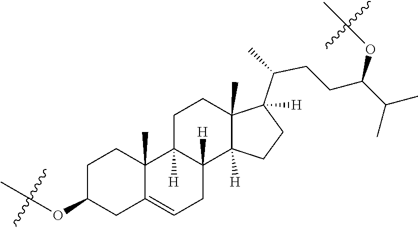 |
| 1435 | 1 | 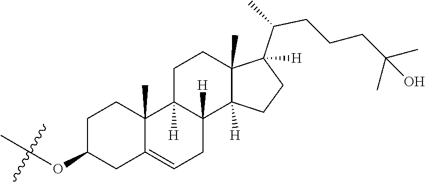 |
| 1436 | 1 | 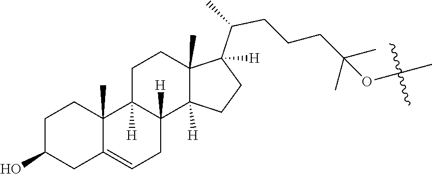 |
| 1437 | 2 | 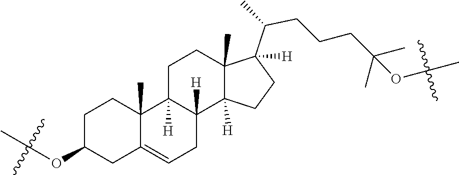 |
| 1438 | 1 | 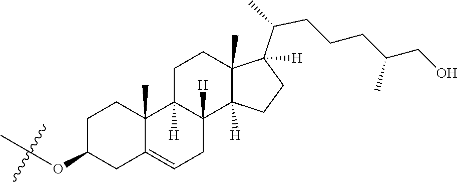 |
| 1439 | 1 | 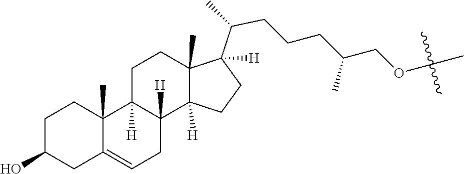 |
| 1440 | 2 | 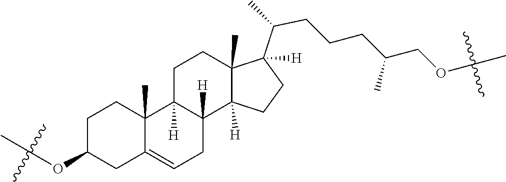 |
| 1441 | 1 | 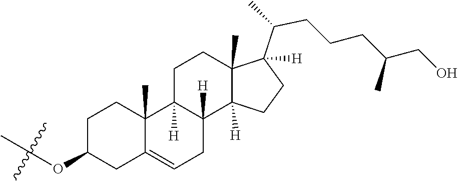 |
| 1442 | 1 | 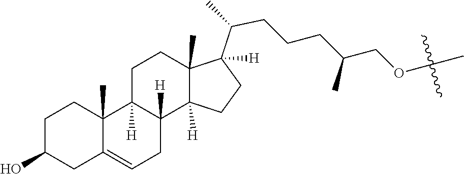 |
| 1443 | 2 | 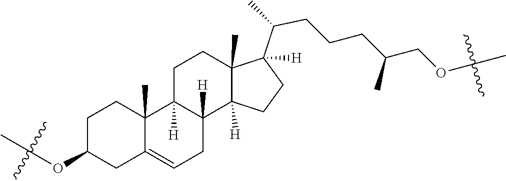 |
| 1444 | 1 | 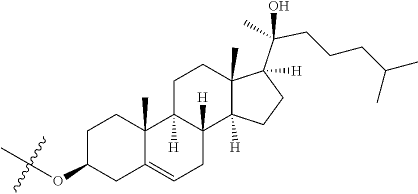 |
| 1445 | 1 | 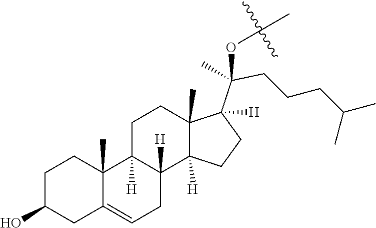 |
| 1446 | 2 | 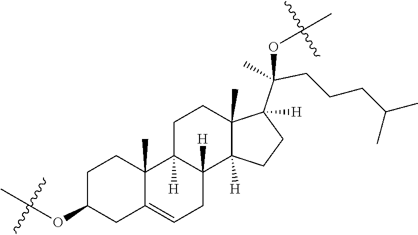 |
| 1447 | 1 | 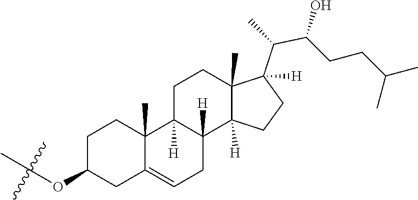 |
| 1448 | 1 | 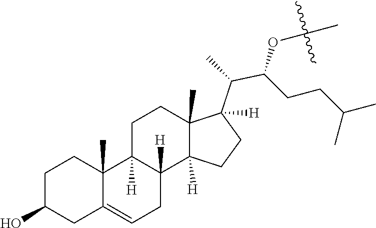 |
| 1449 | 2 | 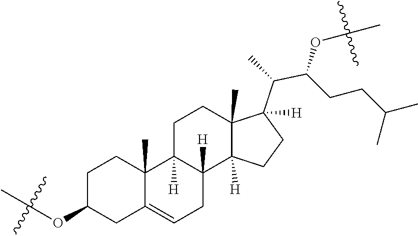 |
| 1450 | 1 | 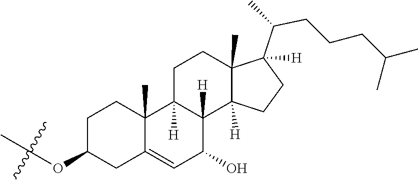 |
| 1451 | 1 | 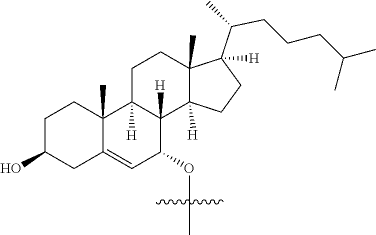 |
| 1452 | 2 | 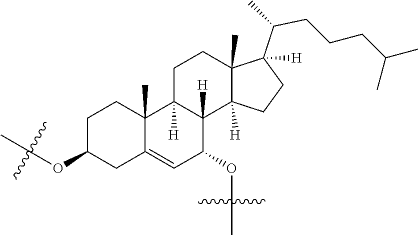 |
| 1453 | 1 | 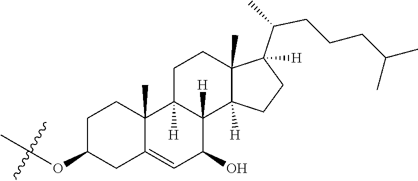 |
| 1454 | 1 | 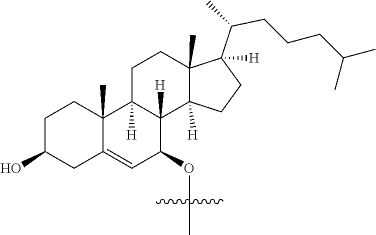 |
| 1455 | 2 | 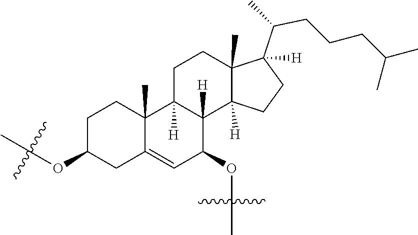 |
| 1456 | 1 | 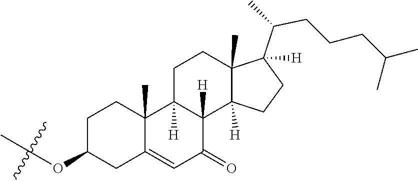 |
| 1457 | 1 | 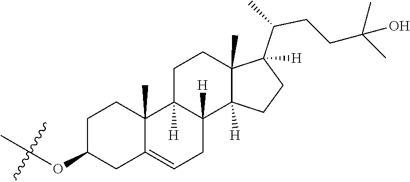 |
| 1458 | 1 | 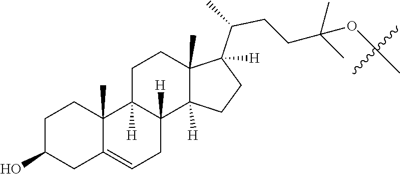 |
| 1459 | 2 | 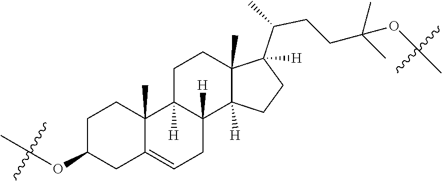 |
| 1460 | 1 | 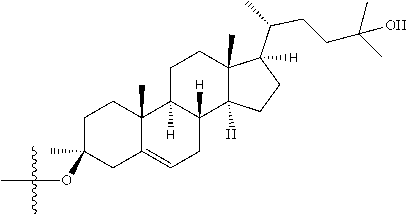 |
| 1461 | 1 | 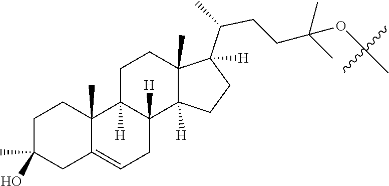 |
| 1462 | 2 | 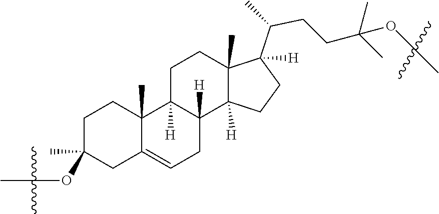 |
| 1463 | 1 | 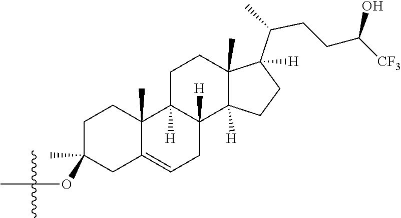 |
| 1464 | 1 | 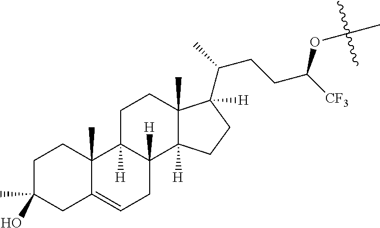 |
| 1465 | 2 | 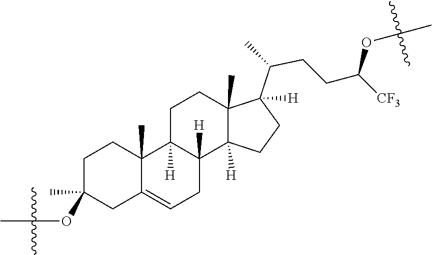 |
| 1466 | 1 | 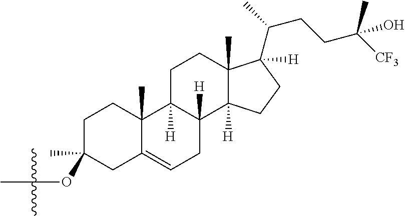 |
| 1467 | 1 | 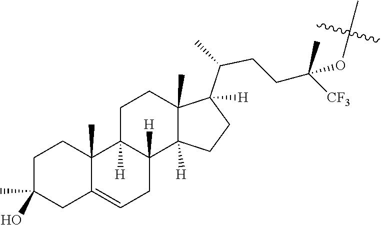 |
| 1468 | 2 | 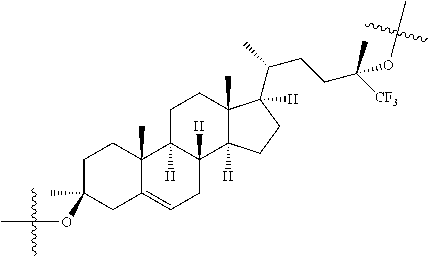 |
| 1469 | 1 | 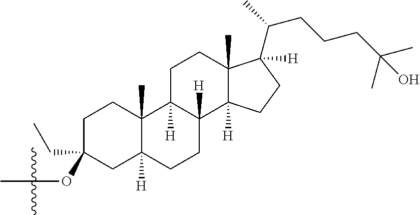 |
| 1470 | 1 | 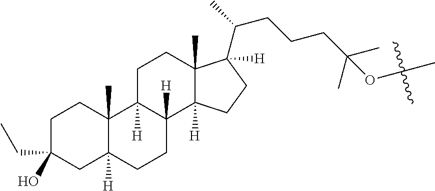 |
| 1471 | 2 | 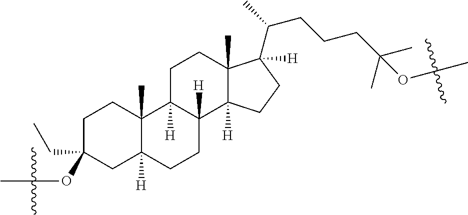 |
| 1472 | 1 | 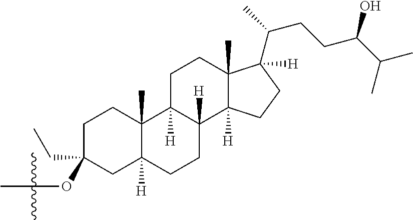 |
| 1473 | 1 | 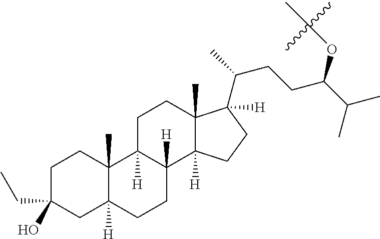 |
| 1474 | 2 | 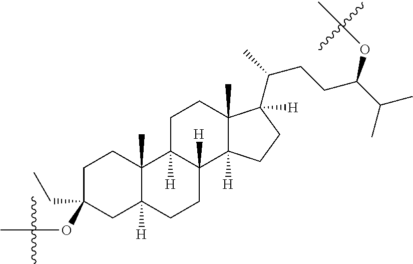 |
| 1475 | 1 | 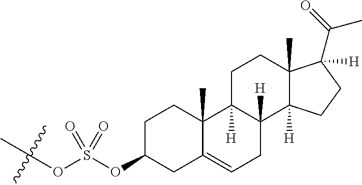 |
| 1476 | 1 | 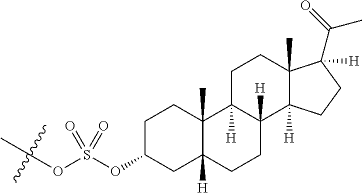 |
| 1477 | 1 | 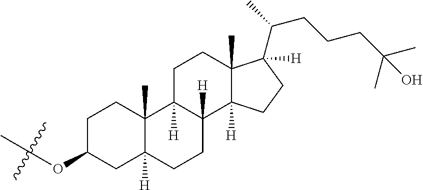 |
| 1478 | 1 | 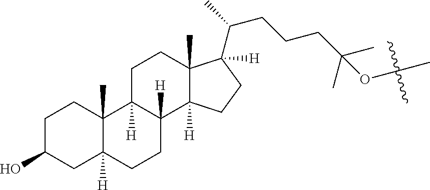 |
| 1479 | 2 | 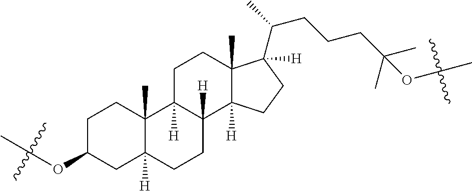 |
| 1480 | 1 | 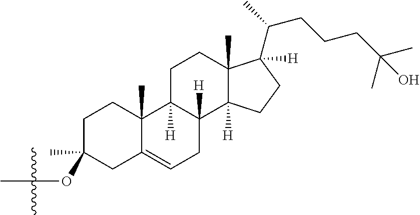 |
| 1481 | 1 | 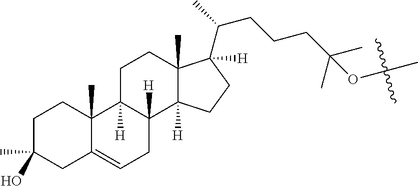 |
| 1482 | 2 | 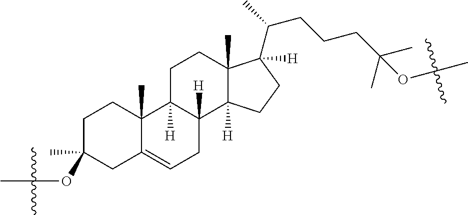 |
| 1483 | 1 | 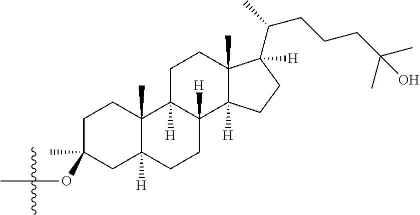 |
| 1484 | 1 | 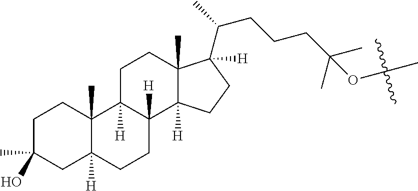 |
| 1485 | 2 | 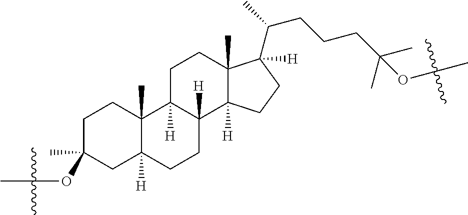 |
| 1486 | 1 | 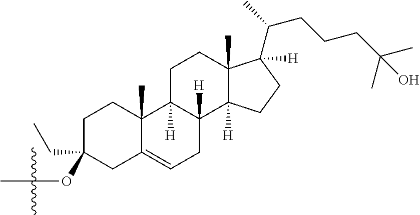 |
| 1487 | 1 | 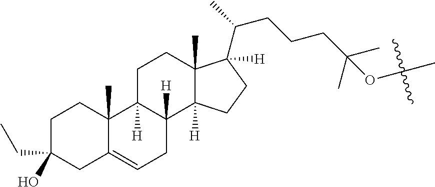 |
| 1488 | 2 | 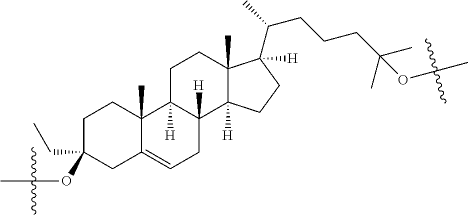 |
| 1489 | 1 | 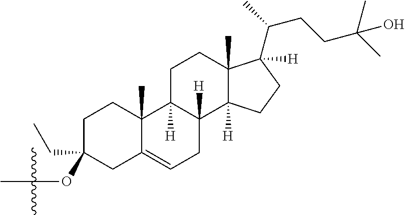 |
| 1490 | 1 | 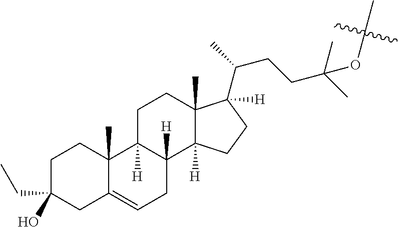 |
| 1491 | 2 | 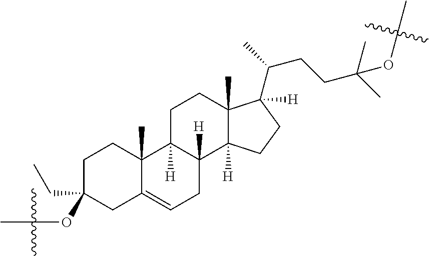 |

In some embodiments, the compound is selected from any of the compounds depicted in in Table 23, pharmaceutically acceptable salts thereof, deuterated variants thereof or combinations thereof.

TABLE 23
Formula (XLIX)

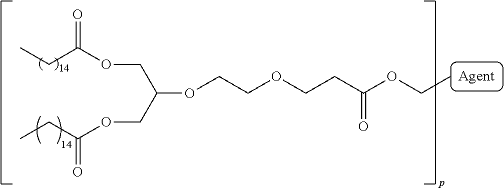

| Cpd. No. | p | 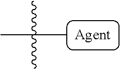 |
|---|---|---|
| 1492 | 1 | 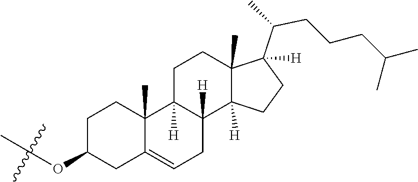 |
| 1493 | 1 | 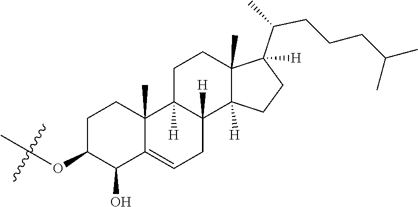 |
| 1494 | 1 | 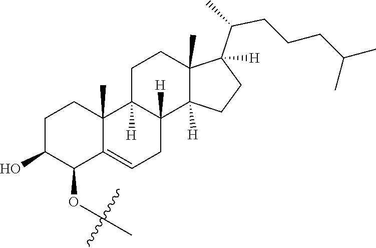 |
| 1495 | 2 | 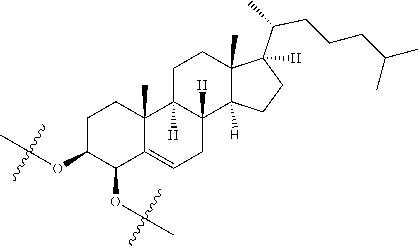 |
| 1496 | 1 | 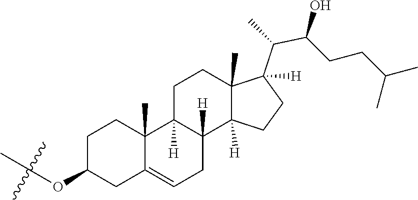 |
| 1497 | 1 | 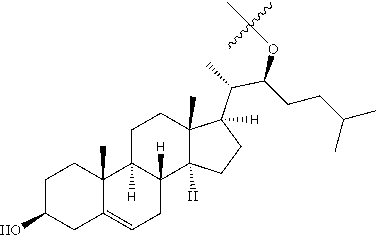 |
| 1498 | 2 | 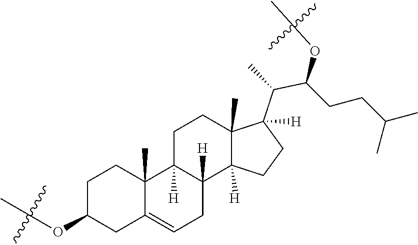 |
| 1499 | 1 | 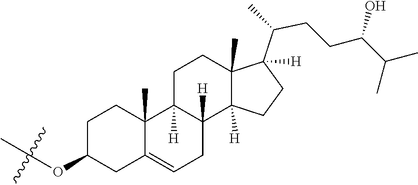 |
| 1500 | 1 | 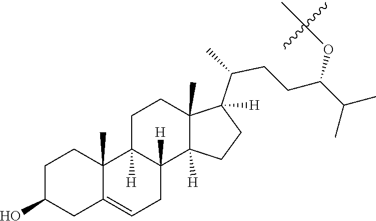 |
| 1501 | 2 | 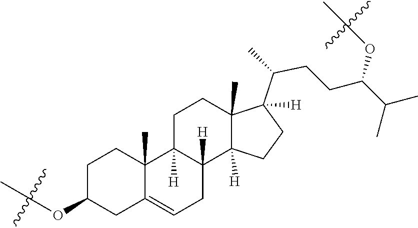 |
| 1502 | 1 | 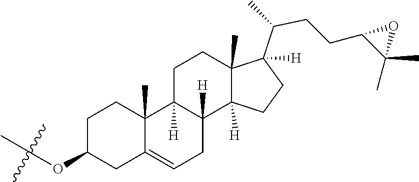 |
| 1503 | 1 | 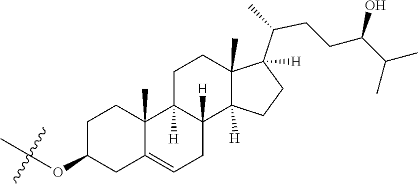 |
| 1504 | 1 | 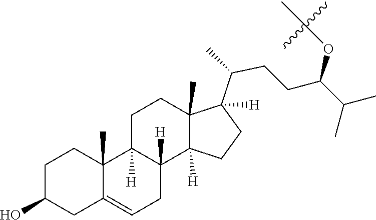 |
| 1505 | 2 | 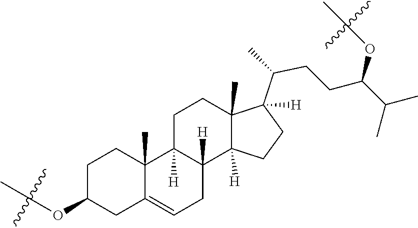 |
| 1506 | 1 | 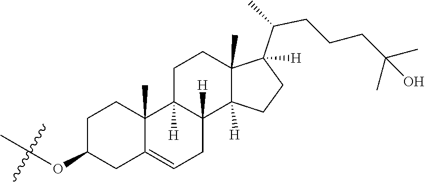 |
| 1507 | 1 | 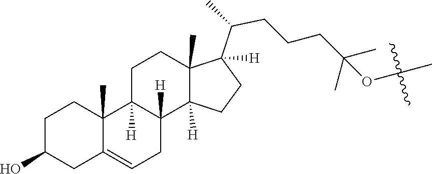 |
| 1508 | 2 | 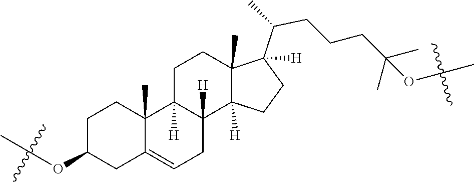 |
| 1509 | 1 | 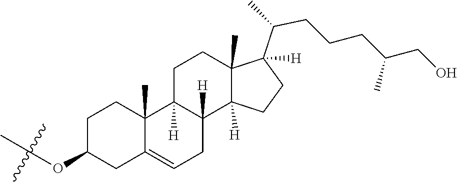 |
| 1510 | 1 | 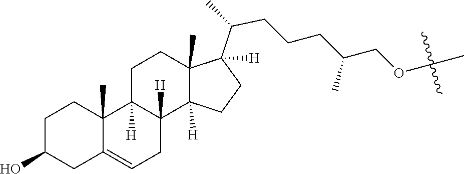 |
| 1511 | 2 | 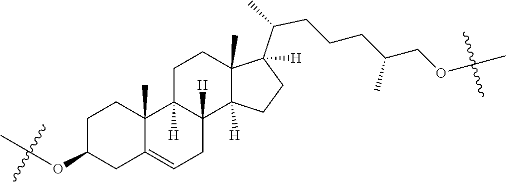 |
| 1512 | 1 | 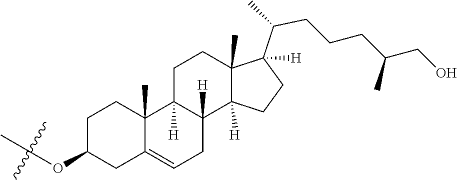 |
| 1513 | 1 | 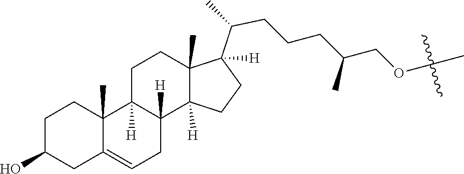 |
| 1514 | 2 | 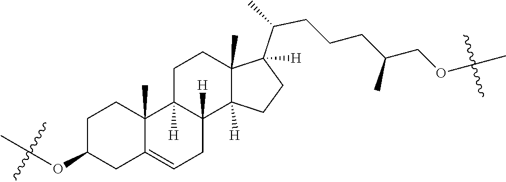 |
| 1515 | 1 | 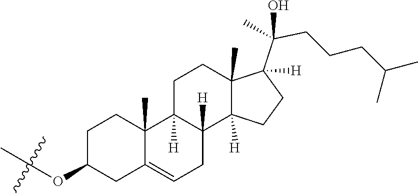 |
| 1516 | 1 | 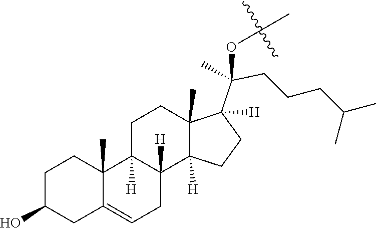 |
| 1517 | 2 | 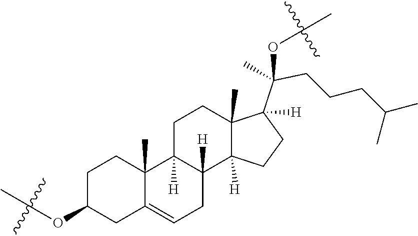 |
| 1518 | 1 | 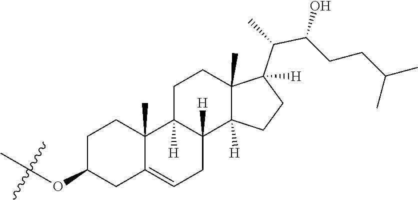 |
| 1519 | 1 | 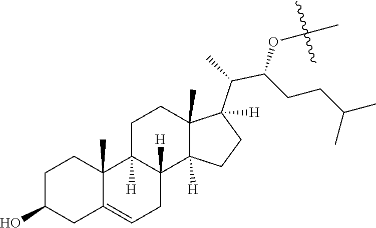 |
| 1520 | 2 | 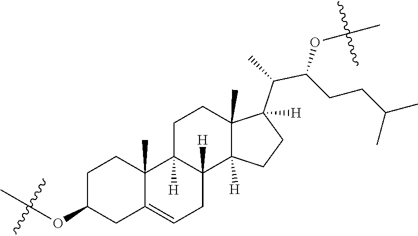 |
| 1521 | 1 | 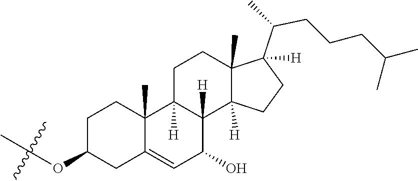 |
| 1522 | 1 | 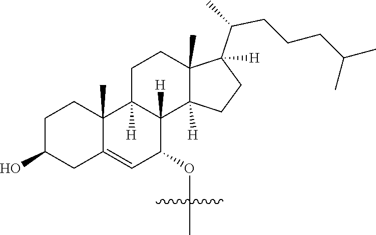 |
| 1523 | 2 | 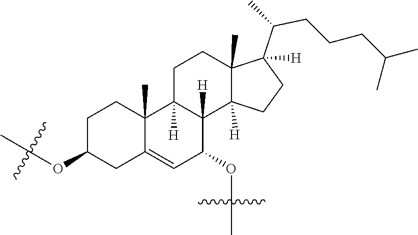 |
| 1524 | 1 | 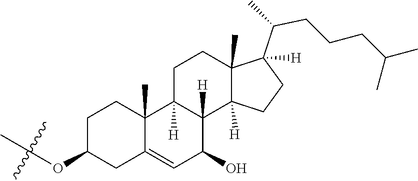 |
| 1525 | 1 | 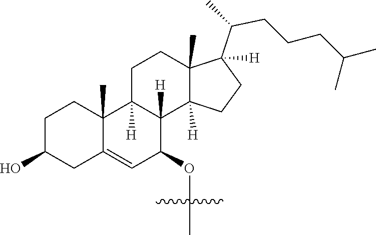 |
| 1526 | 2 | 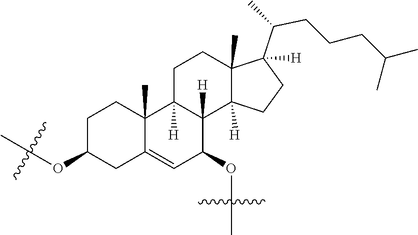 |
| 1527 | 1 | 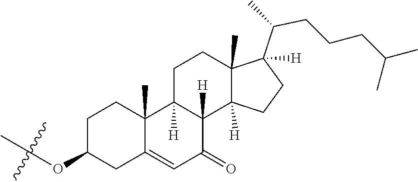 |
| 1528 | 1 | 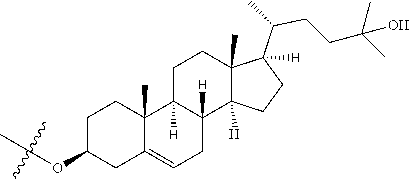 |
| 1529 | 1 | 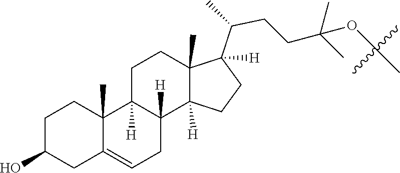 |
| 1530 | 2 | 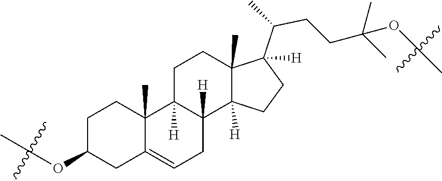 |
| 1531 | 1 | 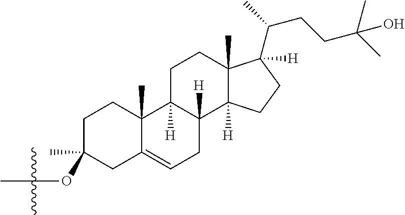 |
| 1532 | 1 | 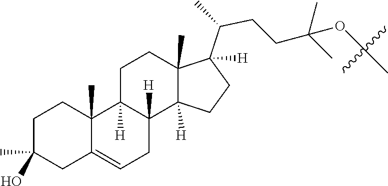 |
| 1533 | 2 | 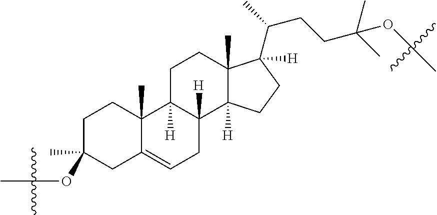 |
| 1534 | 1 | 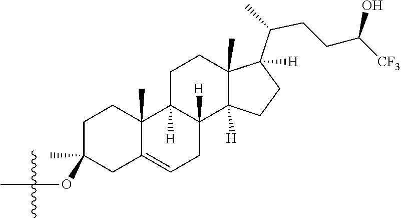 |
| 1535 | 1 | 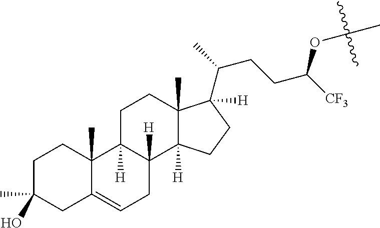 |
| 1536 | 2 | 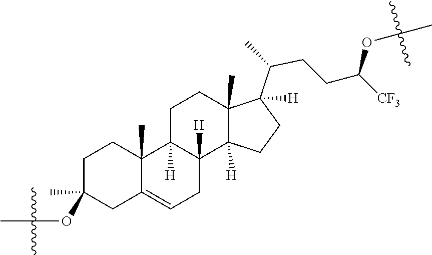 |
| 1537 | 1 | 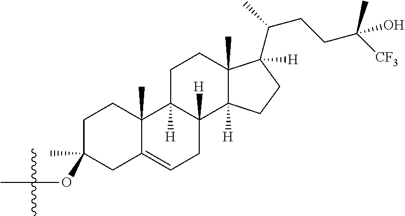 |
| 1538 | 1 | 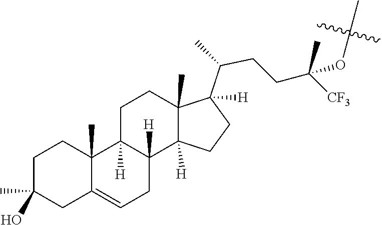 |
| 1539 | 2 | 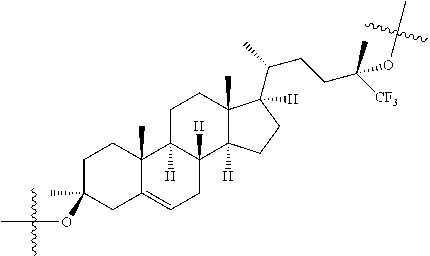 |
| 1540 | 1 | 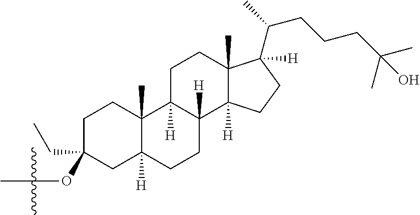 |
| 1541 | 1 | 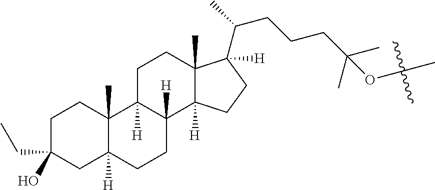 |
| 1542 | 2 | 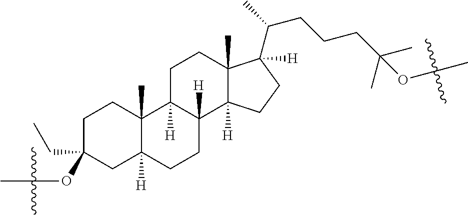 |
| 1543 | 1 | 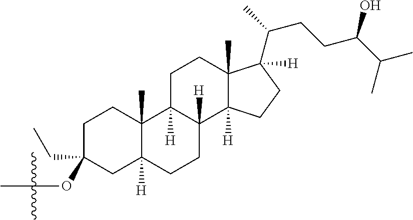 |
| 1544 | 1 | 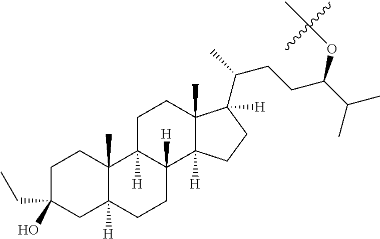 |
| 1545 | 2 | 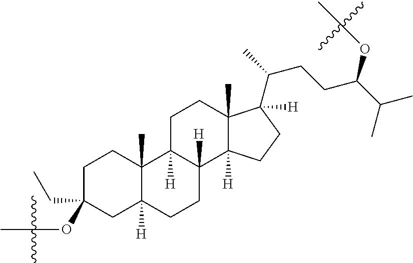 |
| 1546 | 1 | 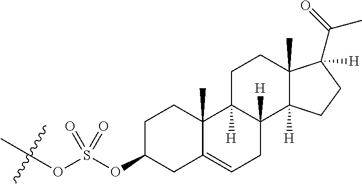 |
| 1547 | 1 | 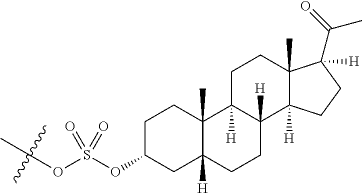 |
| 1548 | 1 | 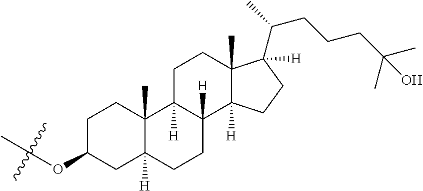 |
| 1549 | 1 | 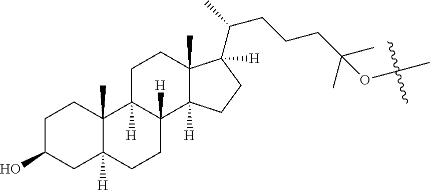 |
| 1550 | 2 | 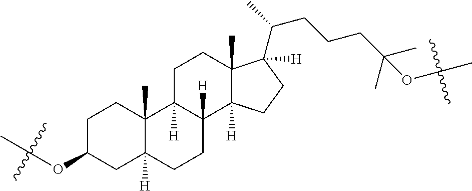 |
| 1551 | 1 | 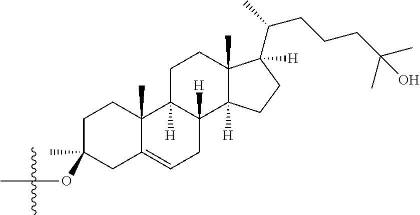 |
| 1552 | 1 | 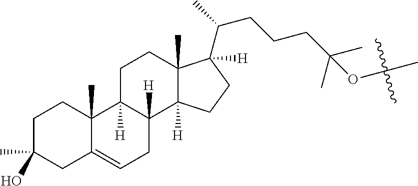 |
| 1553 | 2 | 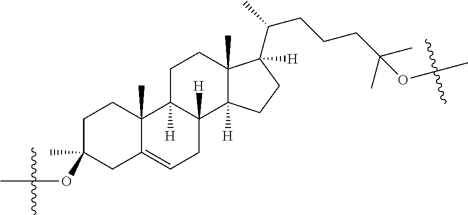 |
| 1554 | 1 | 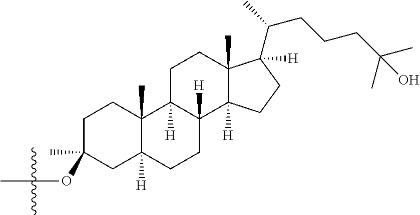 |
| 1555 | 1 | 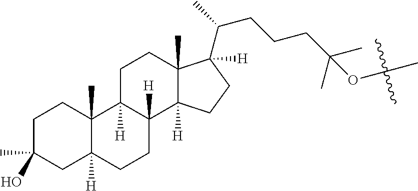 |
| 1556 | 2 | 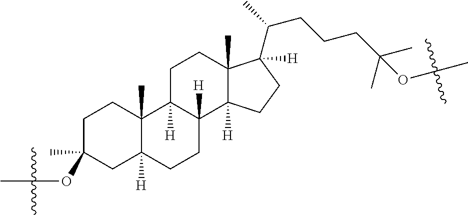 |
| 1557 | 1 | 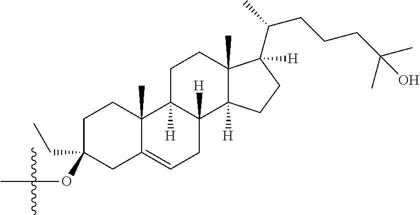 |
| 1558 | 1 | 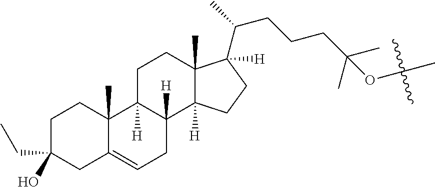 |
| 1559 | 2 | 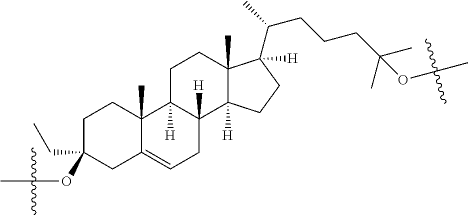 |
| 1560 | 1 | 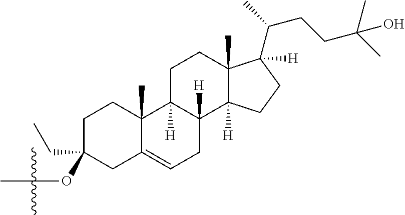 |
| 1561 | 1 | 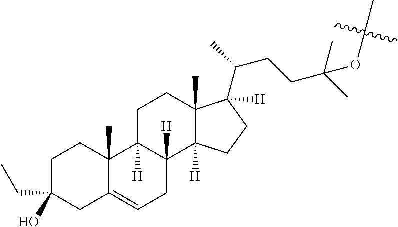 |
| 1562 | 2 | 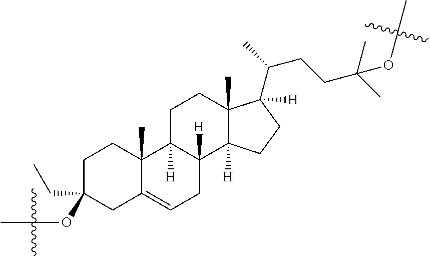 |

In some embodiments, the compound is selected from any of the compounds depicted in in Table 24, pharmaceutically acceptable salts thereof, deuterated variants thereof or combinations thereof.

TABLE 24

| | |
|---|---|
| | Formula (L) |
| 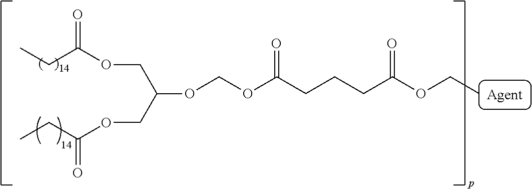 | |

| Cpd. No. | p | 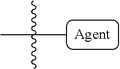 |
|---|---|---|
| 1563 | 1 | 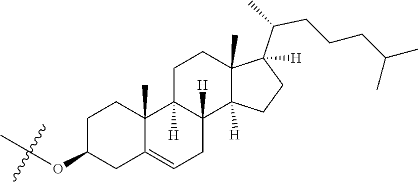 |
| 1564 | 1 | 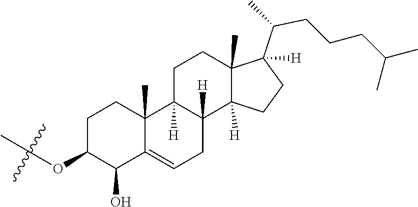 |
| 1565 | 1 | 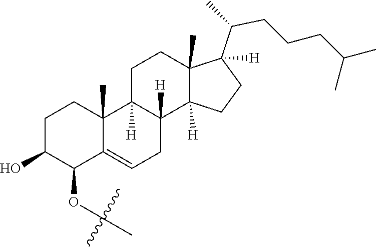 |
| 1566 | 2 | 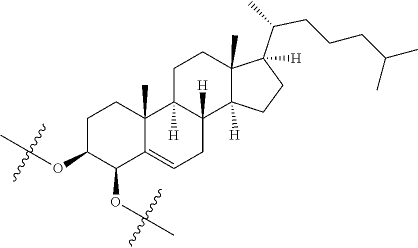 |
| 1567 | 1 | 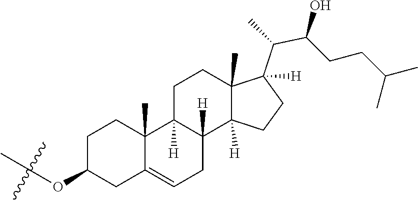 |
| 1568 | 1 | 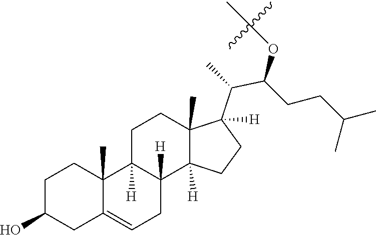 |
| 1569 | 2 | 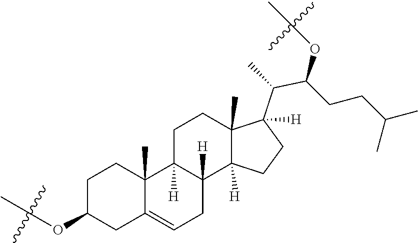 |
| 1570 | 1 | 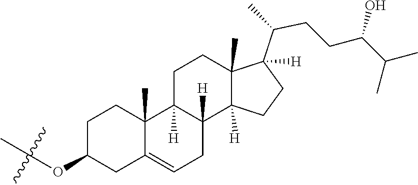 |
| 1571 | 1 | 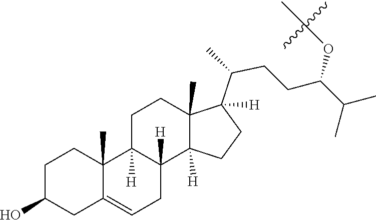 |
| 1572 | 2 | 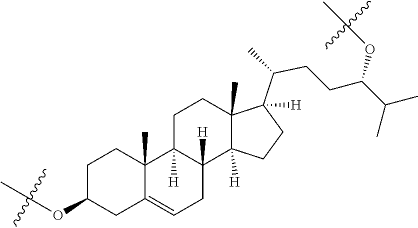 |
| 1573 | 1 | 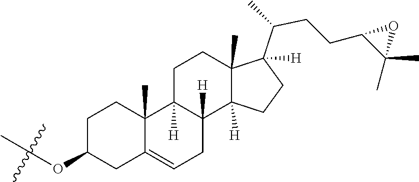 |
| 1574 | 1 | 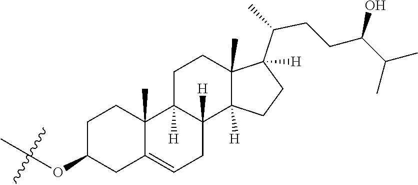 |
| 1575 | 1 | 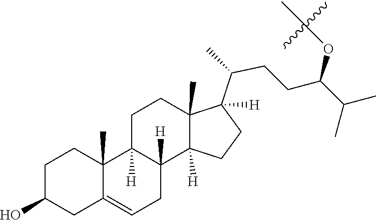 |
| 1576 | 2 | 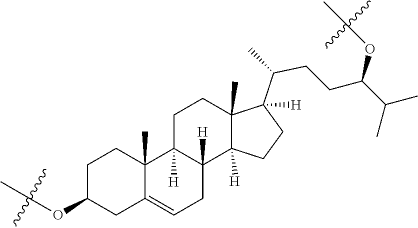 |
| 1577 | 1 | 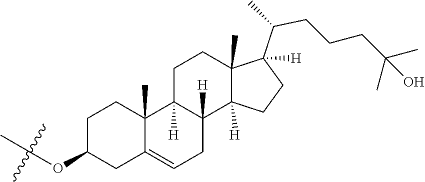 |
| 1578 | 1 | 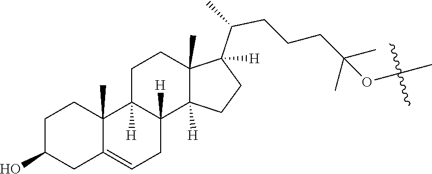 |
| 1579 | 2 | 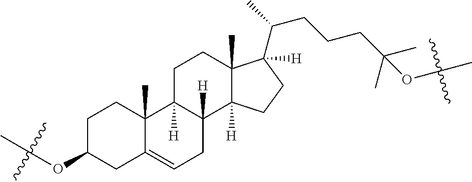 |
| 1580 | 1 | 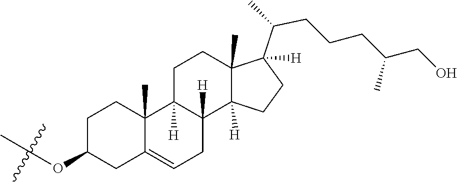 |
| 1581 | 1 | 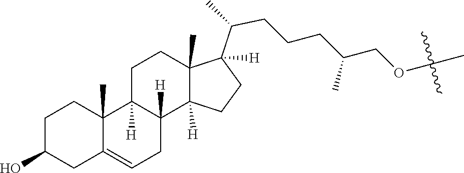 |
| 1582 | 2 | 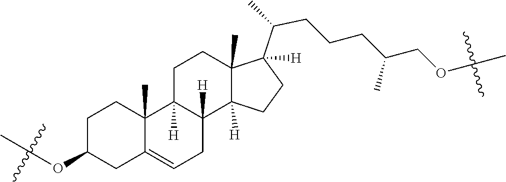 |
| 1583 | 1 | 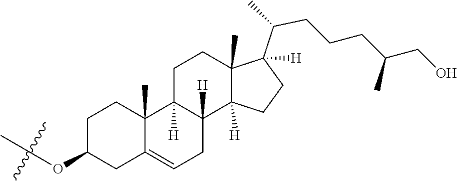 |
| 1584 | 1 | 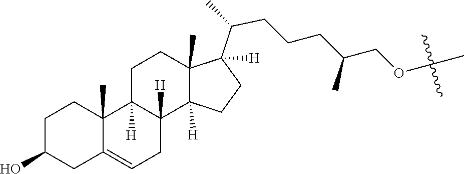 |
| 1585 | 2 | 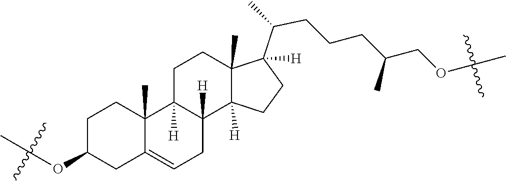 |
| 1586 | 1 | 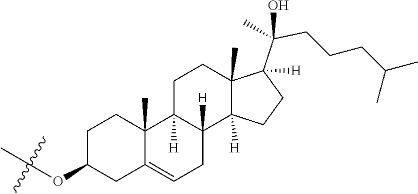 |
| 1587 | 1 | 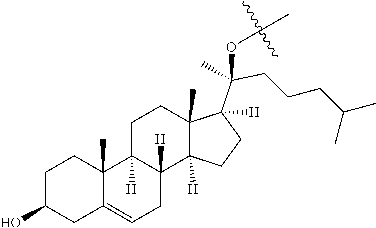 |
| 1588 | 2 | 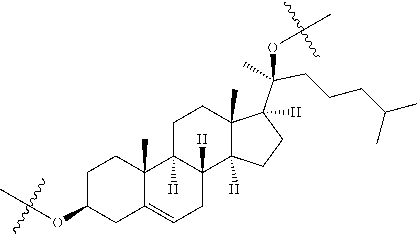 |
| 1589 | 1 | 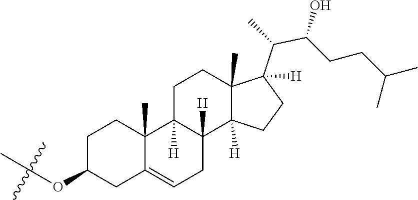 |
| 1590 | 1 | 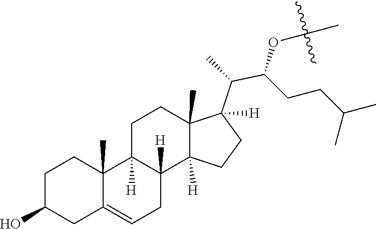 |
| 1591 | 2 | 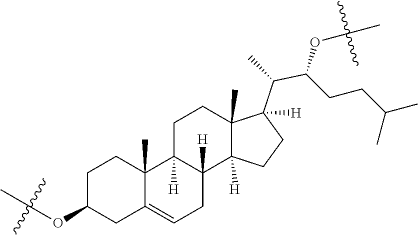 |
| 1592 | 1 | 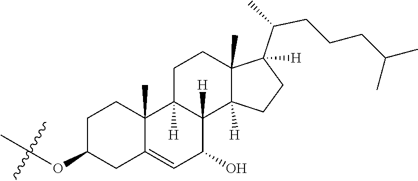 |
| 1593 | 1 | 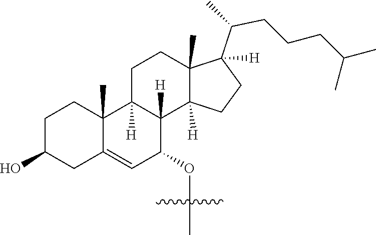 |
| 1594 | 2 | 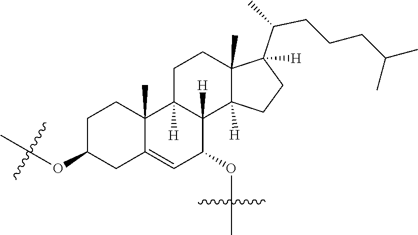 |
| 1595 | 1 | 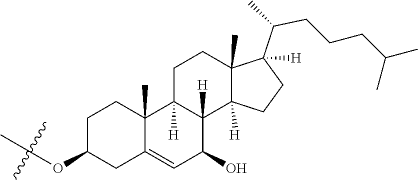 |
| 1596 | 1 | 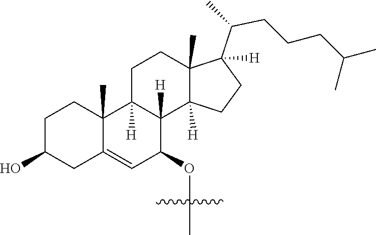 |
| 1597 | 2 | 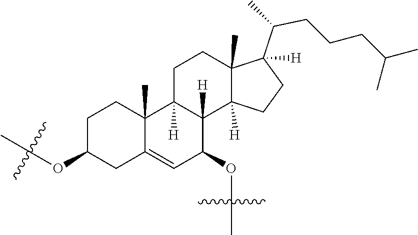 |
| 1598 | 1 | 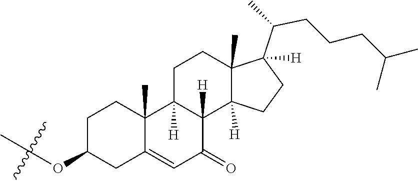 |
| 1599 | 1 | 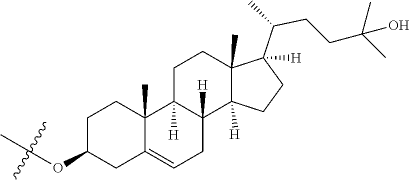 |
| 1600 | 1 | 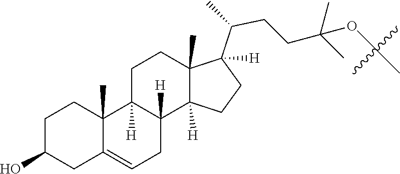 |
| 1601 | 2 | 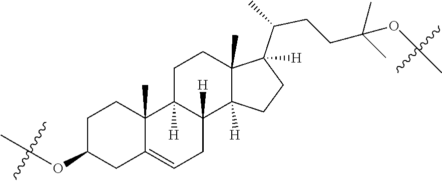 |
| 1602 | 1 | 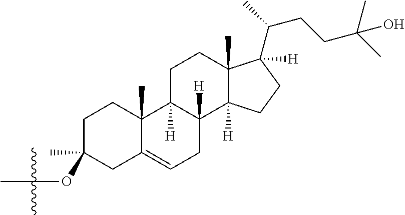 |
| 1603 | 1 | 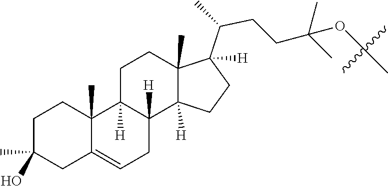 |
| 1604 | 2 | 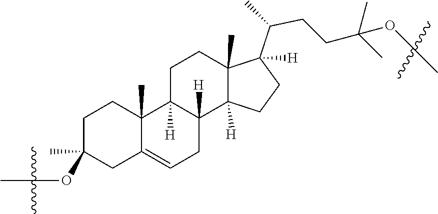 |
| 1605 | 1 | 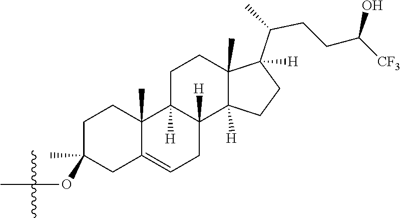 |
| 1606 | 1 | 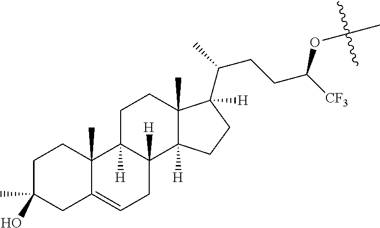 |
| 1607 | 2 | 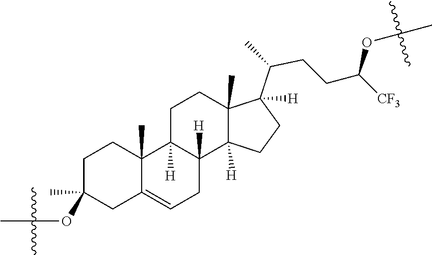 |
| 1608 | 1 | 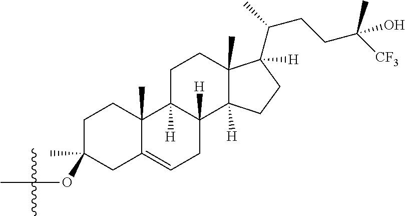 |
| 1609 | 1 | 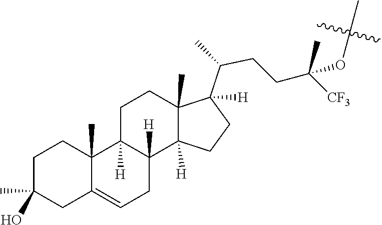 |
| 1610 | 2 | 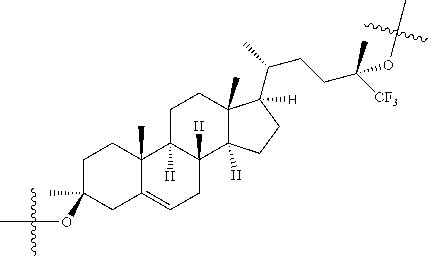 |
| 1611 | 1 | 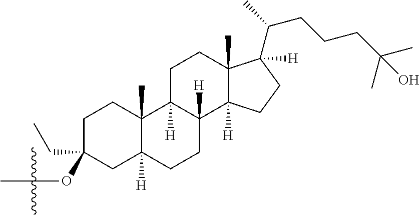 |
| 1612 | 1 | 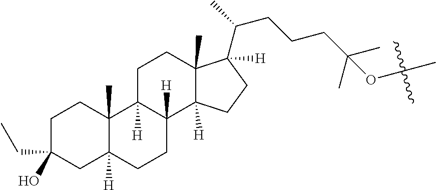 |
| 1613 | 2 | 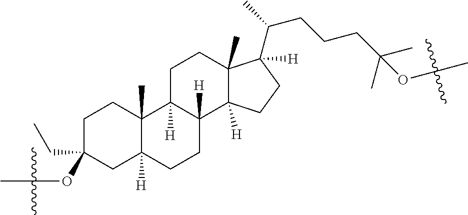 |
| 1614 | 1 | 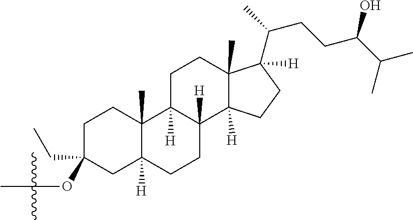 |
| 1615 | 1 | 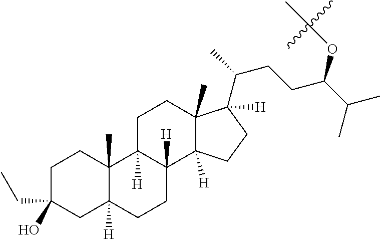 |
| 1616 | 2 | 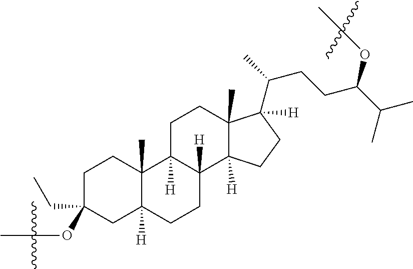 |
| 1617 | 1 | 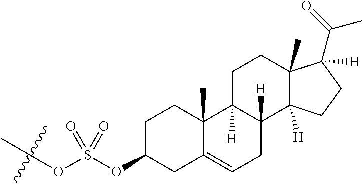 |
| 1618 | 1 | 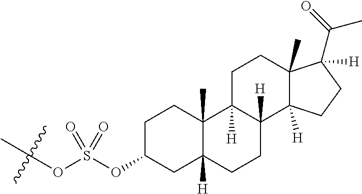 |
| 1619 | 1 | 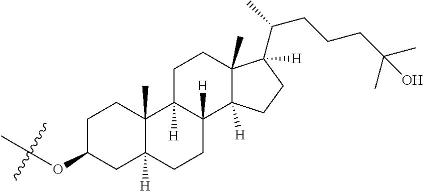 |
| 1620 | 1 | 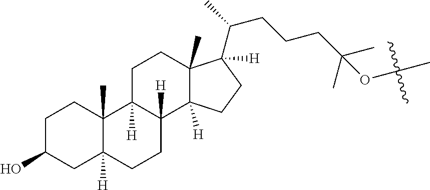 |
| 1621 | 2 | 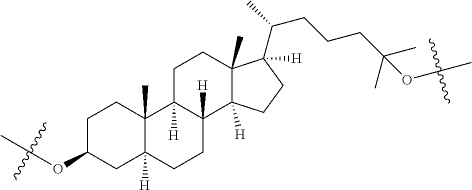 |
| 1622 | 1 | 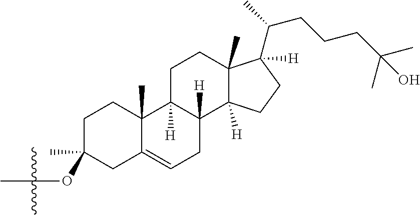 |
| 1623 | 1 | 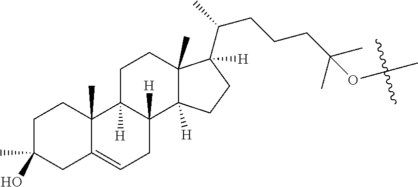 |
| 1624 | 2 | 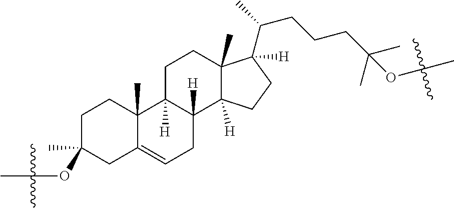 |
| 1625 | 1 | 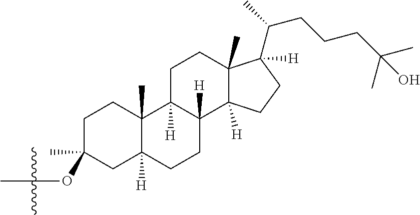 |
| 1626 | 1 | 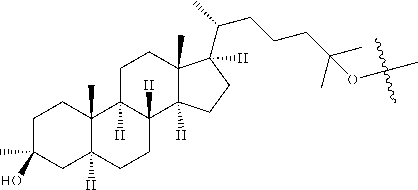 |
| 1627 | 2 | 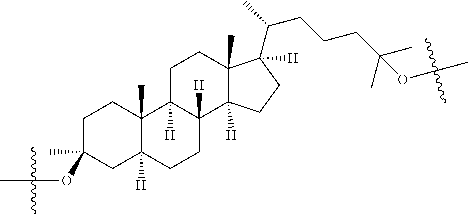 |
| 1628 | 1 | 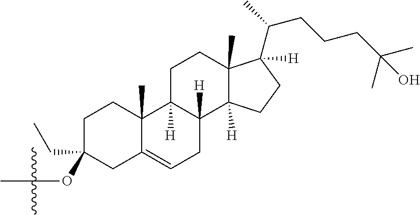 |
| 1629 | 1 | 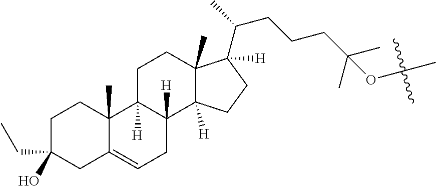 |
| 1630 | 2 | 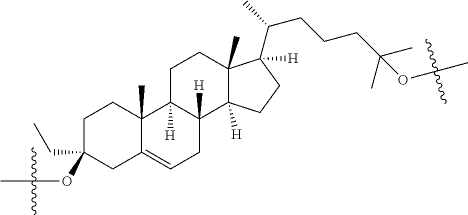 |
| 1631 | 1 | 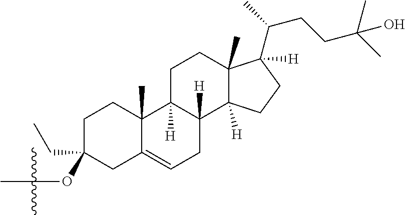 |
| 1632 | 1 | 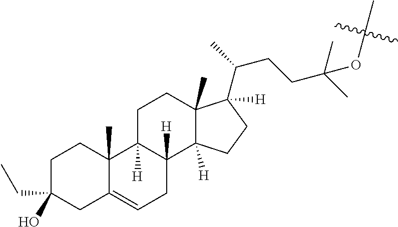 |
| 1633 | 2 | 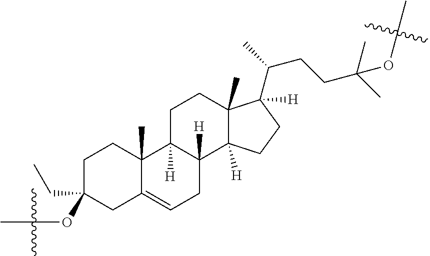 |

In some embodiments, the compound is selected from any of the compounds depicted in in Table 25, pharmaceutically acceptable salts thereof, deuterated variants thereof or combinations thereof.

TABLE 25

| | |
|---|---|
| | Formula (LI) |
| 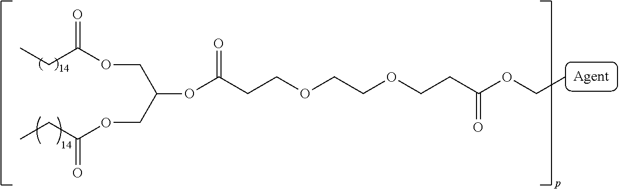 | |

| Cpd. No. | p | 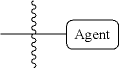 |
|---|---|---|
| 1634 | 1 | 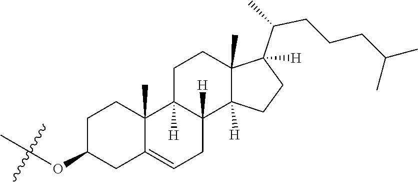 |
| 1635 | 1 | 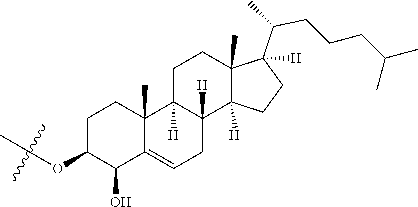 |
| 1636 | 1 | 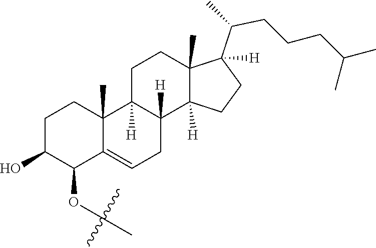 |
| 1637 | 2 | 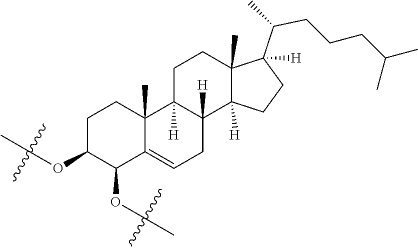 |
| 1638 | 1 | 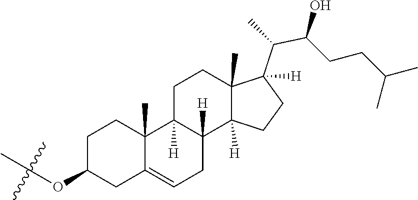 |
| 1639 | 1 | 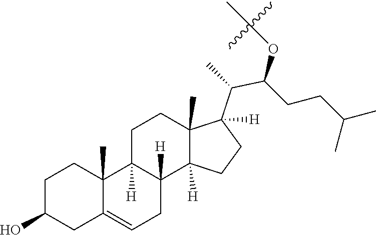 |
| 1640 | 2 | 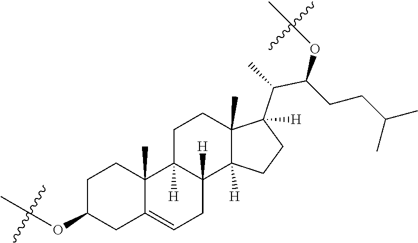 |
| 1641 | 1 | 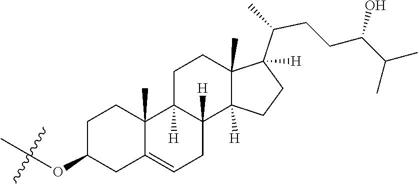 |
| 1642 | 1 | 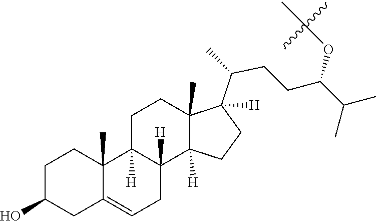 |
| 1643 | 2 | 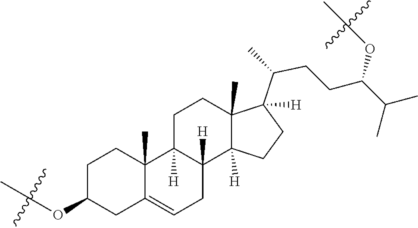 |
| 1644 | 1 | 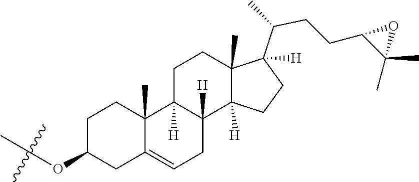 |
| 1645 | 1 | 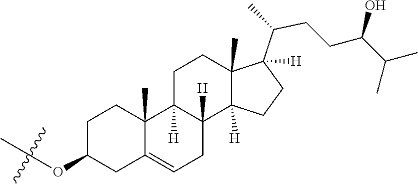 |
| 1646 | 1 | 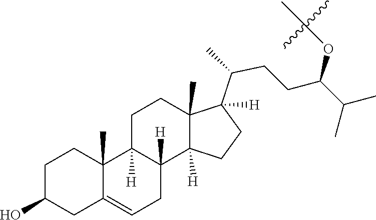 |
| 1647 | 2 | 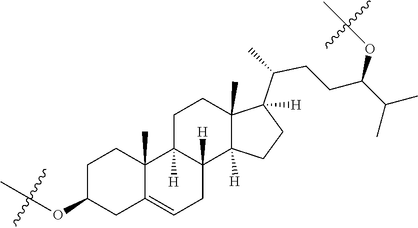 |
| 1648 | 1 | 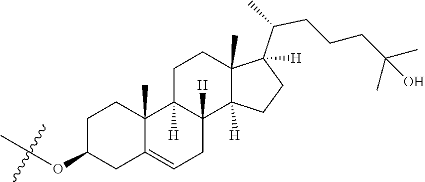 |
| 1649 | 1 | 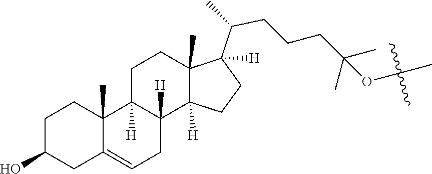 |
| 1650 | 2 | 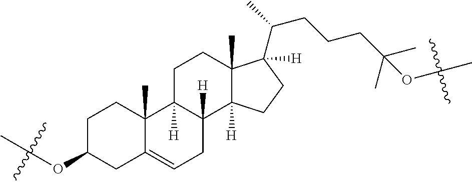 |
| 1651 | 1 | 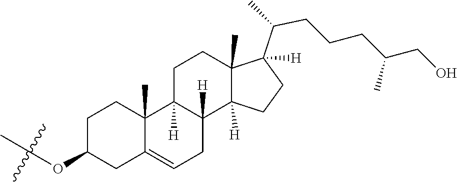 |
| 1652 | 1 | 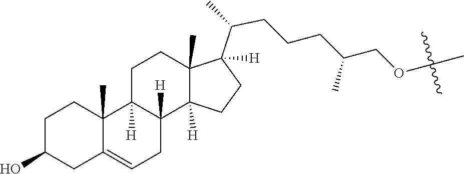 |
| 1653 | 2 | 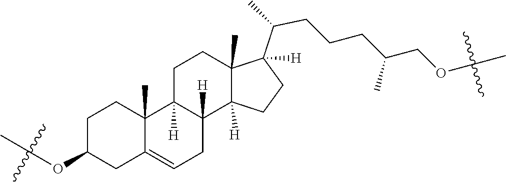 |
| 1654 | 1 | 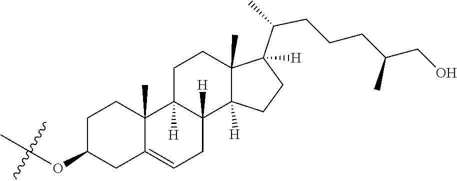 |
| 1655 | 1 | 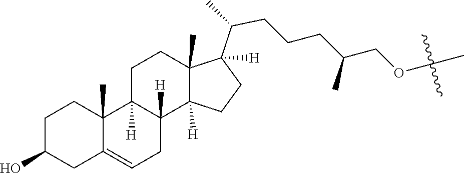 |
| 1656 | 2 | 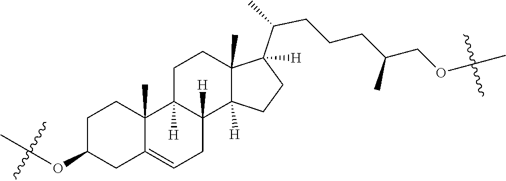 |
| 1657 | 1 | 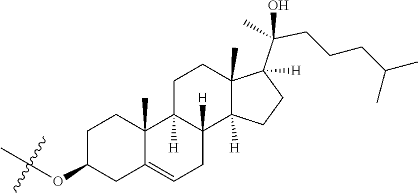 |
| 1658 | 1 | 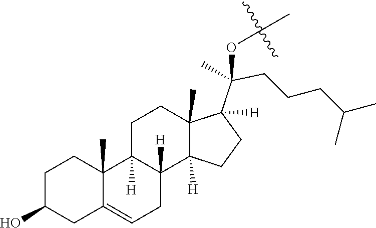 |
| 1659 | 2 | 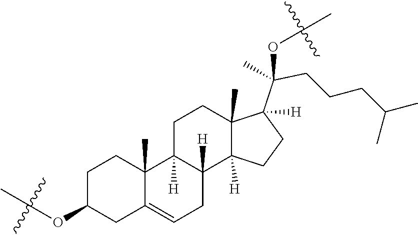 |
| 1660 | 1 | 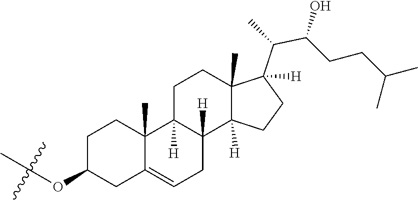 |
| 1661 | 1 | 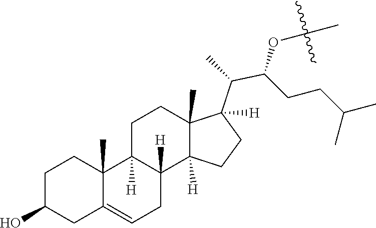 |
| 1662 | 2 | 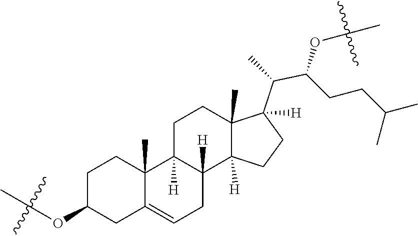 |
| 1663 | 1 | 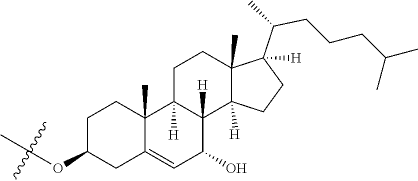 |
| 1664 | 1 | 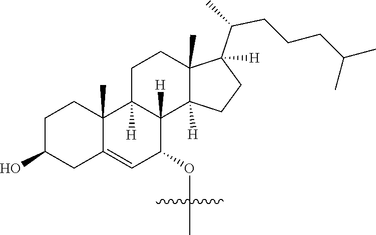 |
| 1665 | 2 | 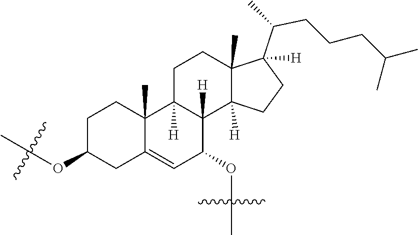 |
| 1666 | 1 | 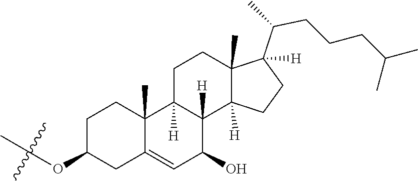 |
| 1667 | 1 | 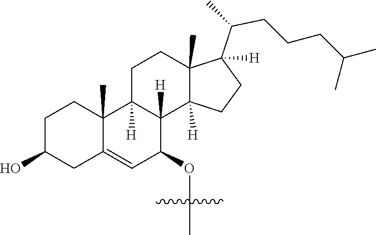 |
| 1668 | 2 | 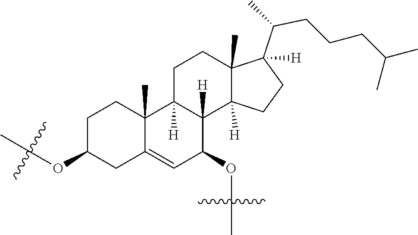 |
| 1669 | 1 | 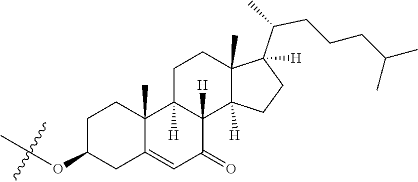 |
| 1670 | 1 | 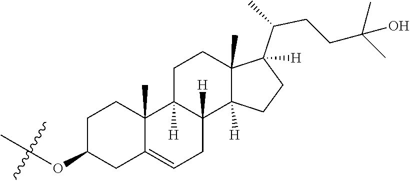 |
| 1671 | 1 | 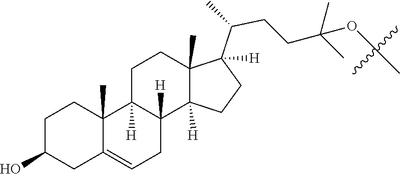 |
| 1672 | 2 | 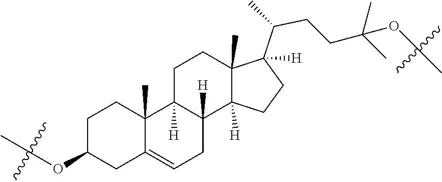 |
| 1673 | 1 | 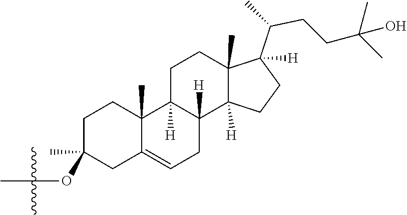 |
| 1674 | 1 | 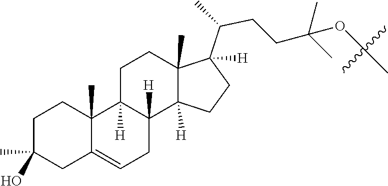 |
| 1675 | 2 | 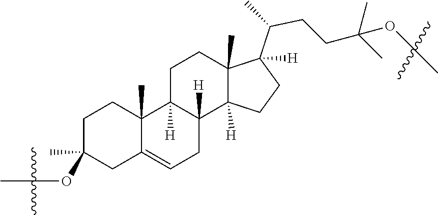 |
| 1676 | 1 | 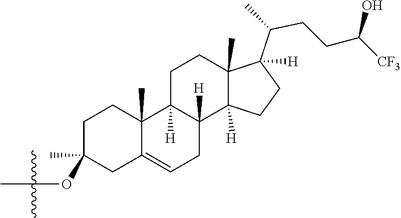 |
| 1677 | 1 | 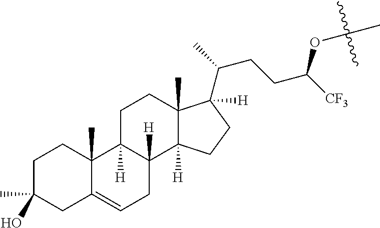 |
| 1678 | 2 | 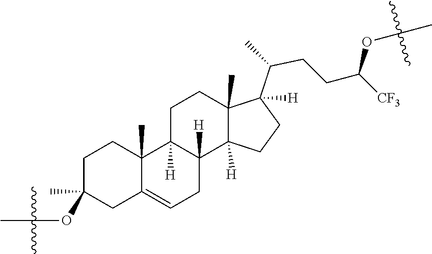 |
| 1679 | 1 | 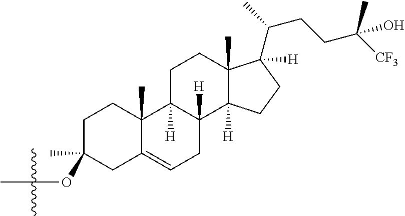 |
| 1680 | 1 | 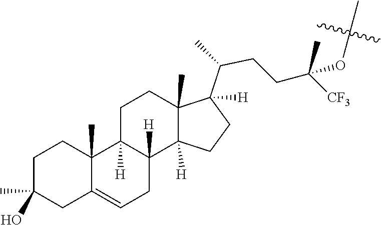 |
| 1681 | 2 | 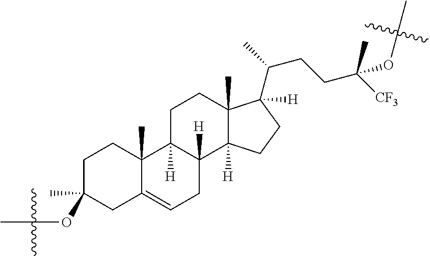 |
| 1682 | 1 | 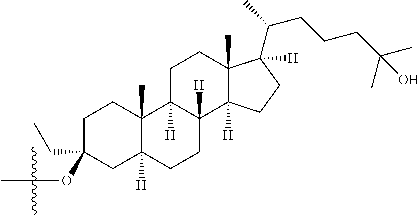 |
| 1683 | 1 | 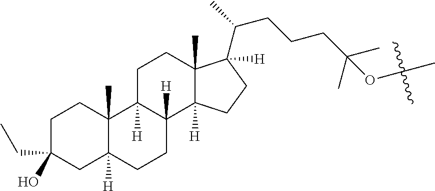 |
| 1684 | 2 | 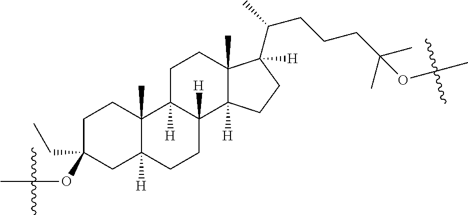 |
| 1685 | 1 | 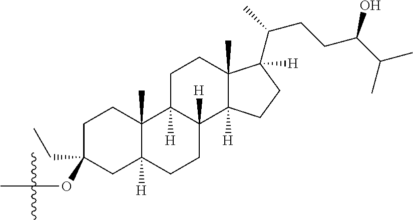 |
| 1686 | 1 | 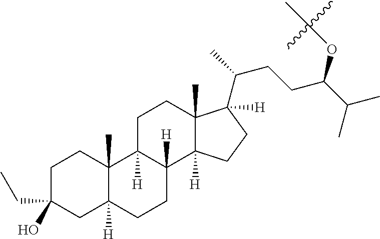 |
| 1687 | 2 | 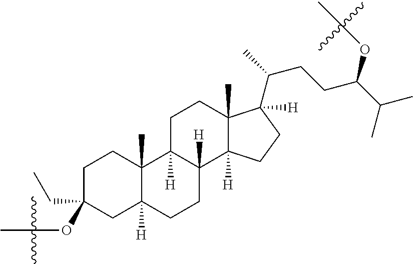 |
| 1688 | 1 | 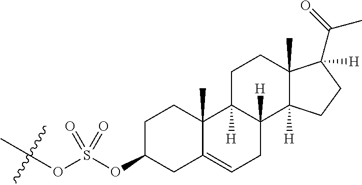 |
| 1689 | 1 | 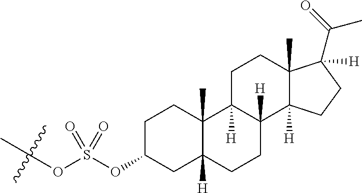 |
| 1690 | 1 | 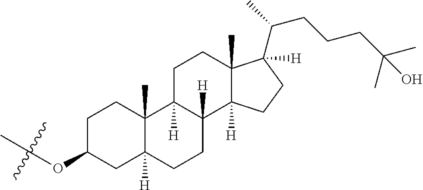 |
| 1691 | 1 | 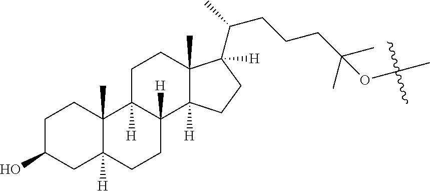 |
| 1692 | 2 | 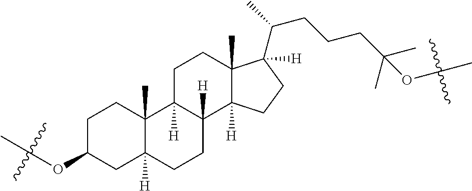 |
| 1693 | 1 | 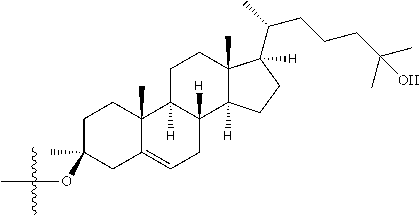 |
| 1694 | 1 | 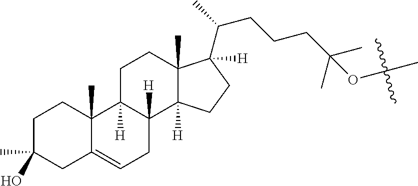 |
| 1695 | 2 | 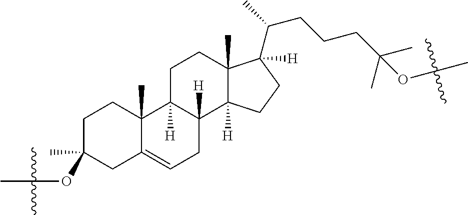 |
| 1696 | 1 | 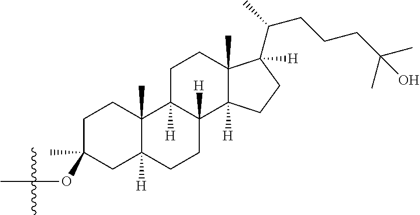 |
| 1697 | 1 | 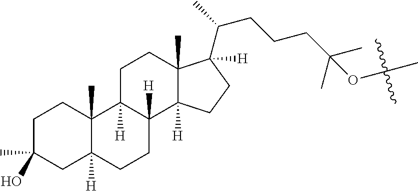 |
| 1698 | 2 | 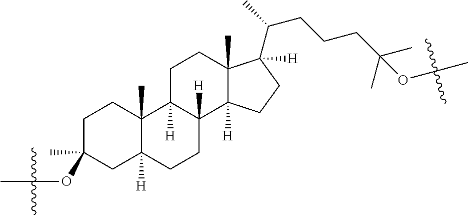 |
| 1699 | 1 | 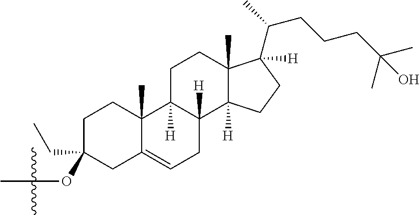 |
| 1700 | 1 | 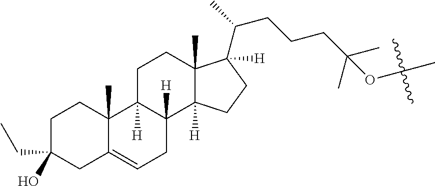 |
| 1701 | 2 | 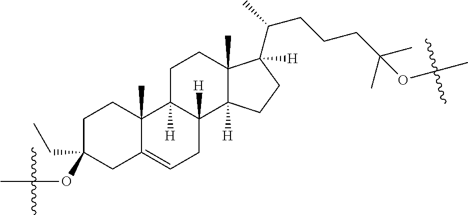 |
| 1702 | 1 | 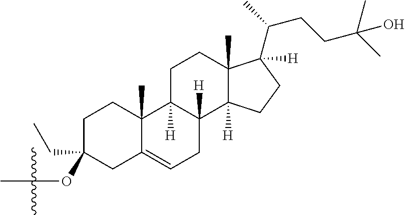 |
| 1703 | 1 | 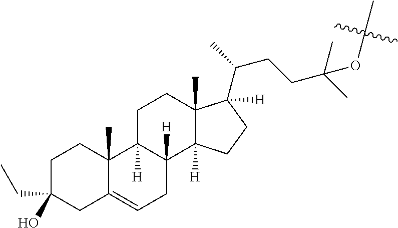 |
| 1704 | 2 | 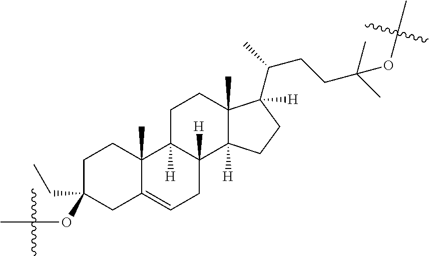 |

In some embodiments, the compound is selected from any of the compounds depicted in in Table 26, pharmaceutically acceptable salts thereof, deuterated variants thereof or combinations thereof.

TABLE 26

| | |
|---|---|
| | Formula (LII) |
| 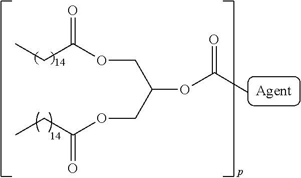 | |

| Cpd. No. | p | 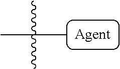 |
|---|---|---|
| 1705 | 1 | 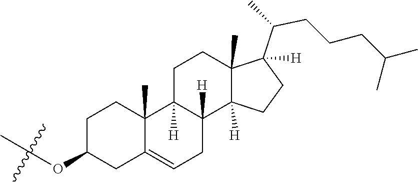 |
| 1706 | 1 | 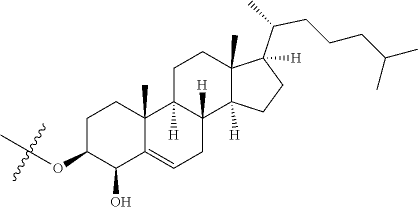 |
| 1707 | 1 | 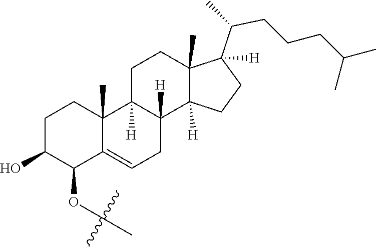 |
| 1708 | 2 | 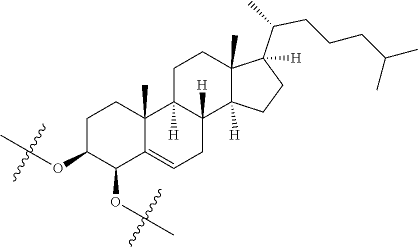 |
| 1709 | 1 | 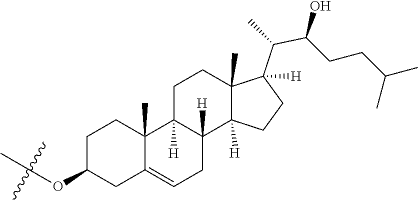 |
| 1710 | 1 | 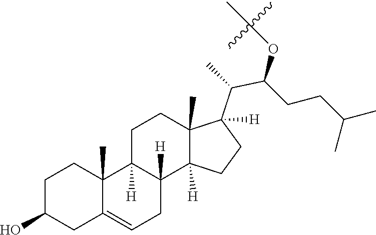 |
| 1711 | 2 | 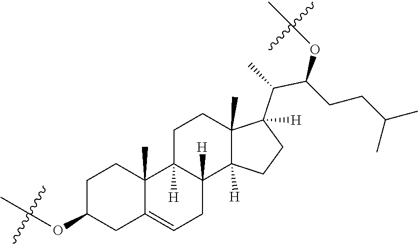 |
| 1712 | 1 | 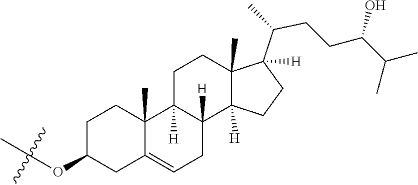 |
| 1713 | 1 | 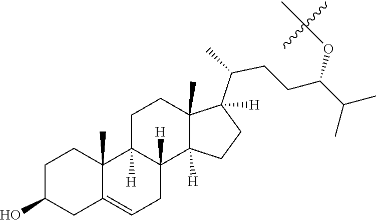 |
| 1714 | 2 | 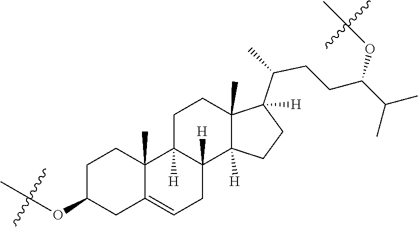 |
| 1715 | 1 | 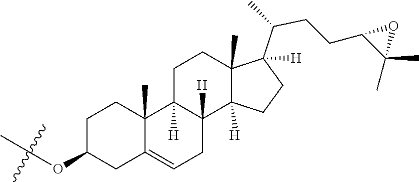 |
| 1716 | 1 | 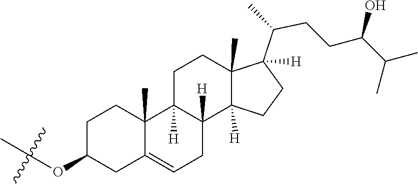 |
| 1717 | 1 | 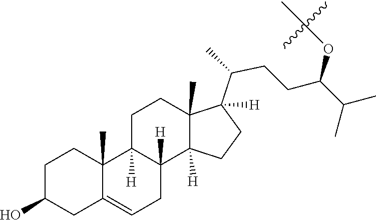 |
| 1718 | 2 | 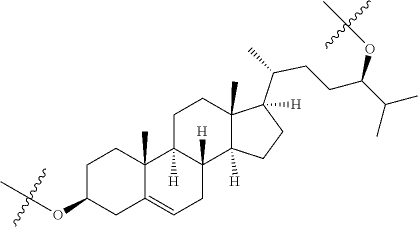 |
| 1719 | 1 | 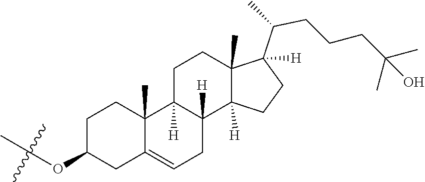 |
| 1720 | 1 | 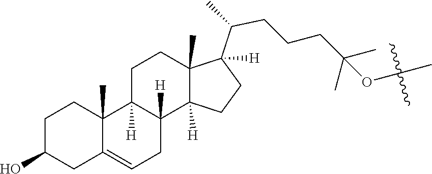 |
| 1721 | 2 | 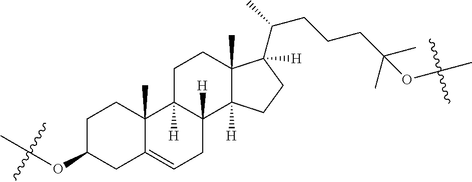 |
| 1722 | 1 | 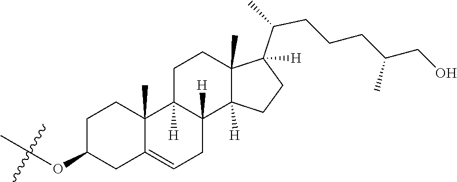 |
| 1723 | 1 | 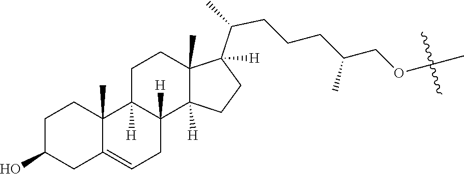 |
| 1724 | 2 | 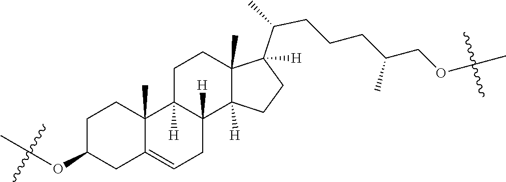 |
| 1725 | 1 | 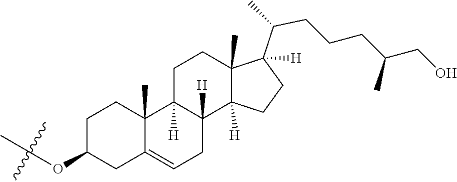 |
| 1726 | 1 | 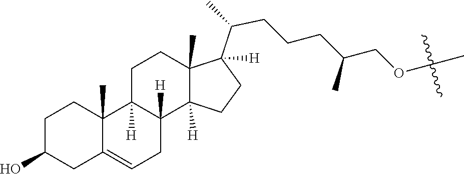 |
| 1727 | 2 | 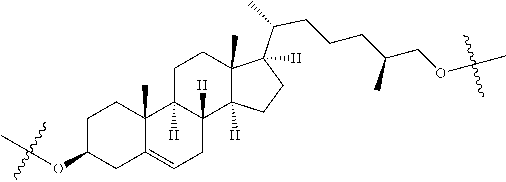 |
| 1728 | 1 | 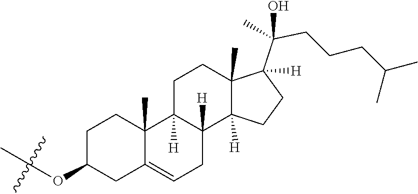 |
| 1729 | 1 | 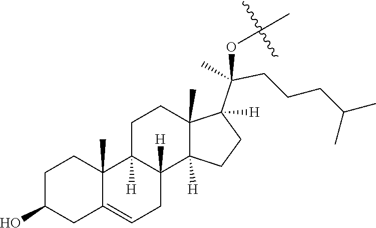 |
| 1730 | 2 | 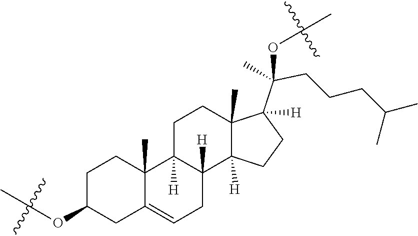 |
| 1731 | 1 | 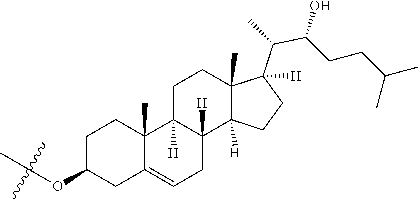 |
| 1732 | 1 | 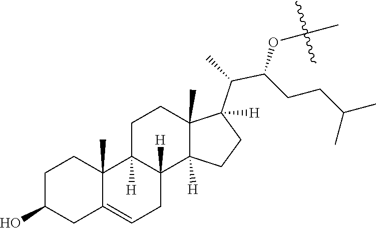 |
| 1733 | 2 | 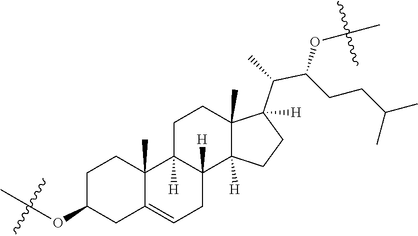 |
| 1734 | 1 | 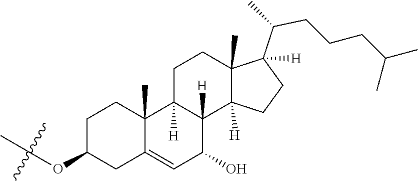 |
| 1735 | 1 | 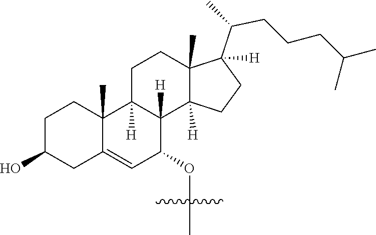 |
| 1736 | 2 | 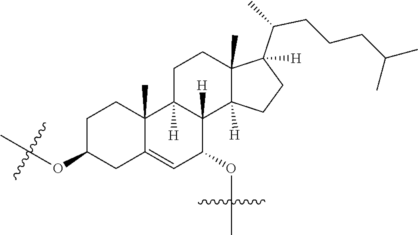 |
| 1737 | 1 | 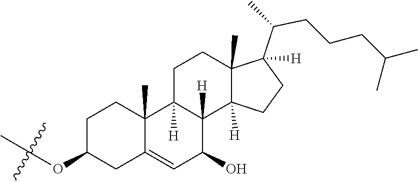 |
| 1738 | 1 | 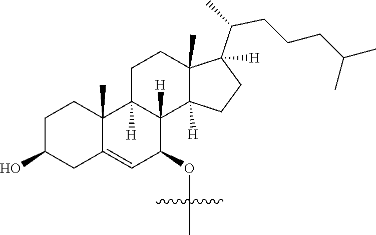 |
| 1739 | 2 | 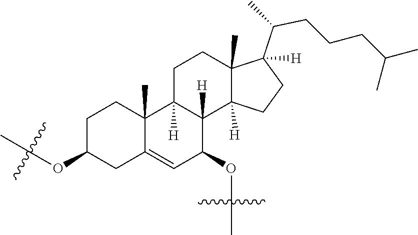 |
| 1740 | 1 | 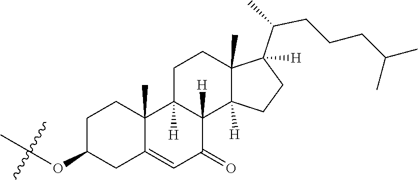 |
| 1741 | 1 | 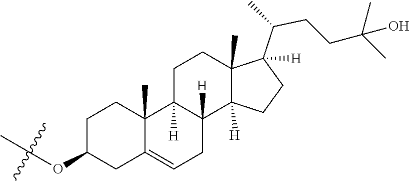 |
| 1742 | 1 | 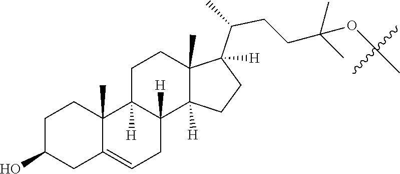 |
| 1743 | 2 | 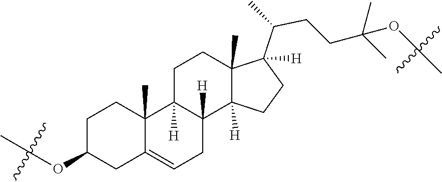 |
| 1744 | 1 | 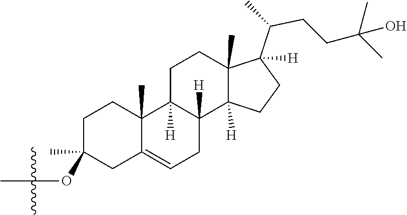 |
| 1745 | 1 | 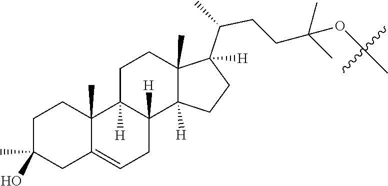 |
| 1746 | 2 | 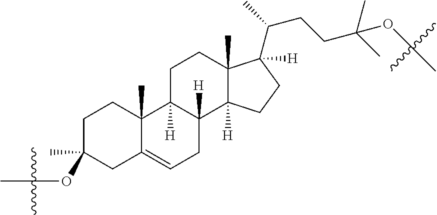 |
| 1747 | 1 | 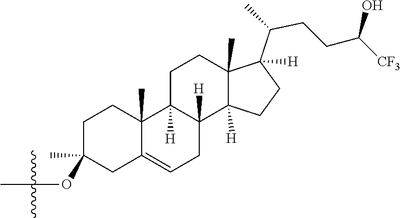 |
| 1748 | 1 | 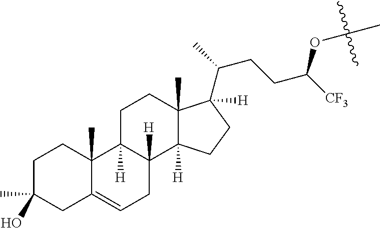 |
| 1749 | 2 | 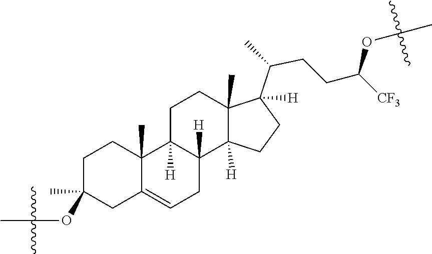 |
| 1750 | 1 | 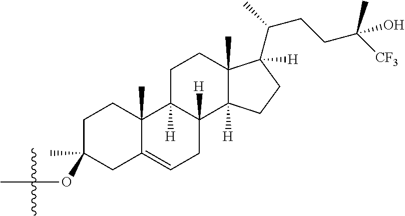 |
| 1751 | 1 | 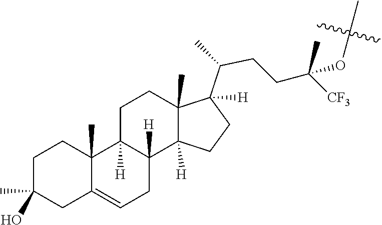 |
| 1752 | 2 | 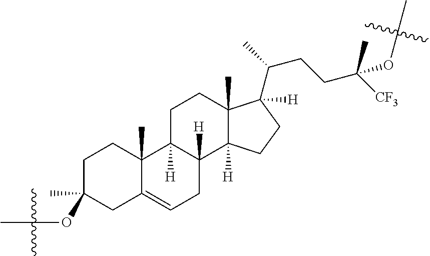 |
| 1753 | 1 | 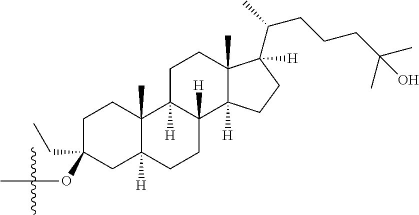 |
| 1754 | 1 | 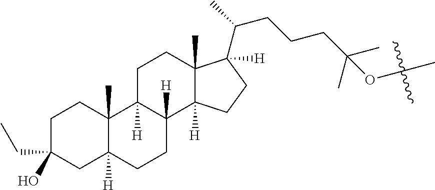 |
| 1755 | 2 | 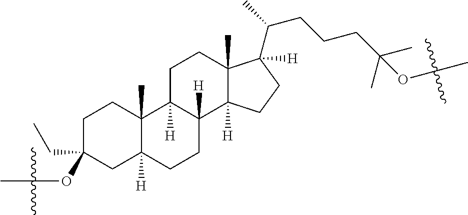 |
| 1756 | 1 | 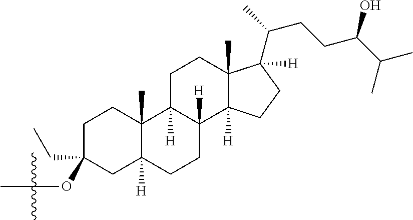 |
| 1757 | 1 | 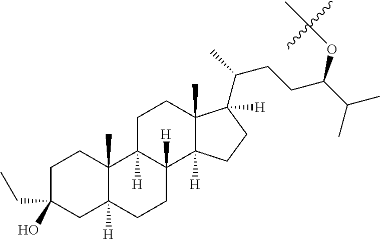 |
| 1758 | 2 | 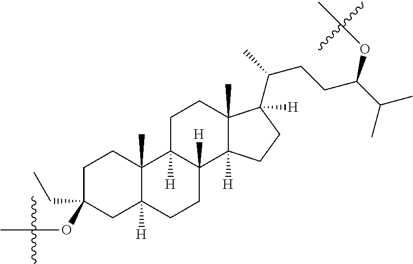 |
| 1759 | 1 | 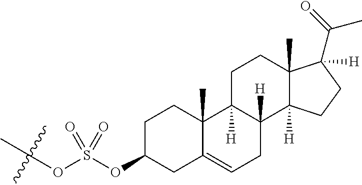 |
| 1760 | 1 | 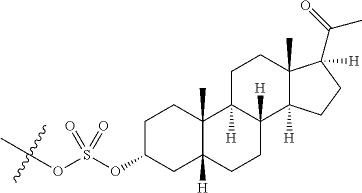 |
| 1761 | 1 | 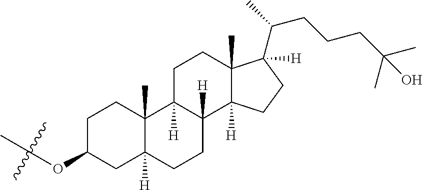 |
| 1762 | 1 | 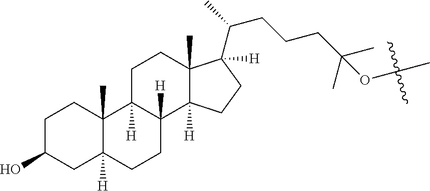 |
| 1763 | 2 | 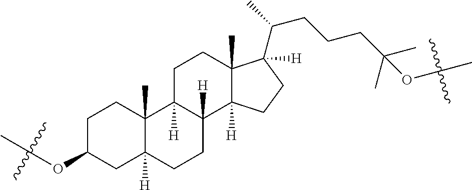 |
| 1764 | 1 | 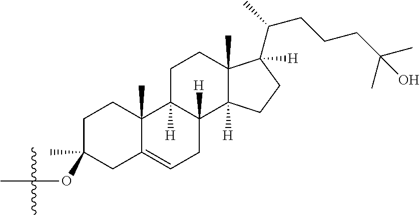 |
| 1765 | 1 | 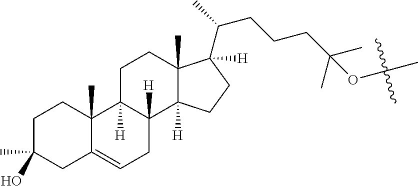 |
| 1766 | 2 | 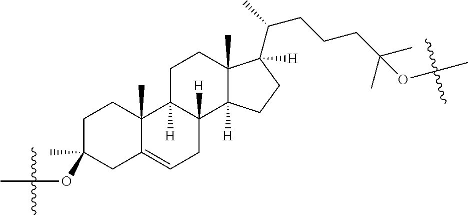 |
| 1767 | 1 | 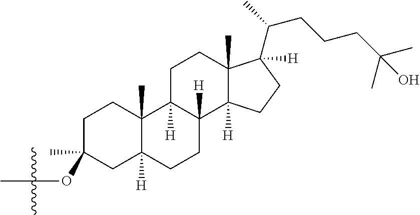 |
| 1768 | 1 | 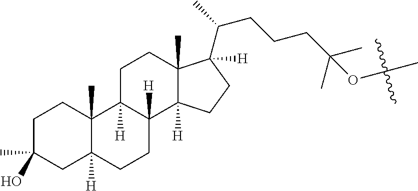 |
| 1769 | 2 | 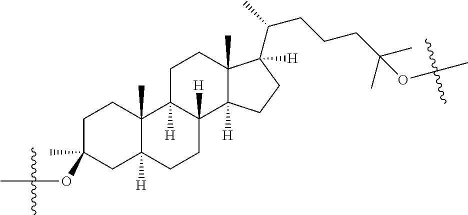 |
| 1770 | 1 | 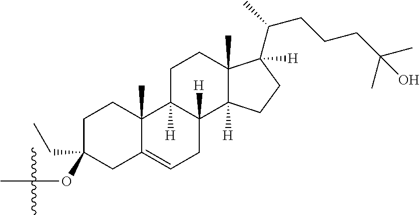 |
| 1771 | 1 | 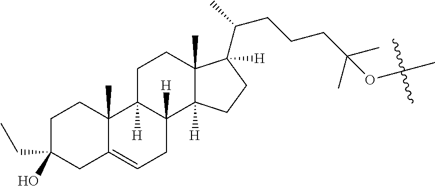 |
| 1772 | 2 | 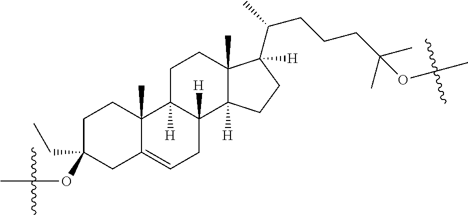 |
| 1773 | 1 | 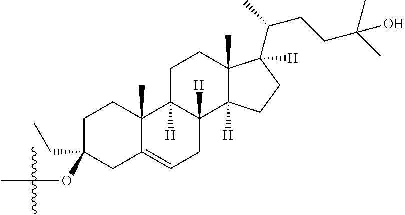 |
| 1774 | 1 | 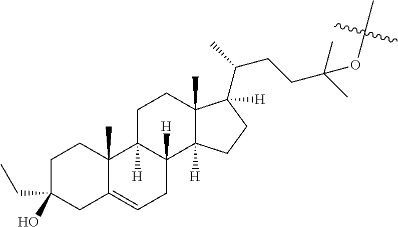 |
| 1775 | 2 | 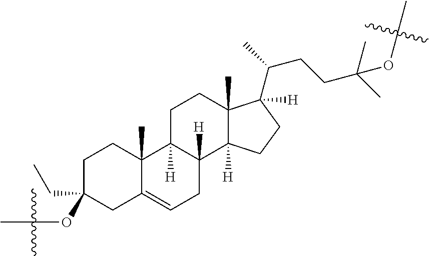 |

In some embodiments, the compound is selected from any of the compounds depicted in in Table 27, pharmaceutically acceptable salts thereof, deuterated variants thereof or combinations thereof.

TABLE 27

| | |
|---|---|
| | Formula (LIII) |
| 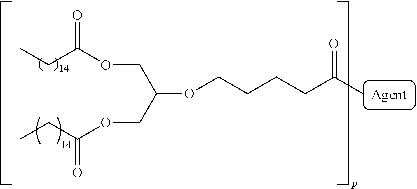 | |

| Cpd. No. | p | 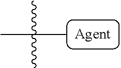 |
|---|---|---|
| 1776 | 1 | 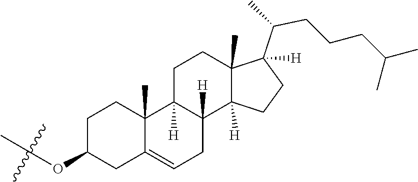 |
| 1777 | 1 | 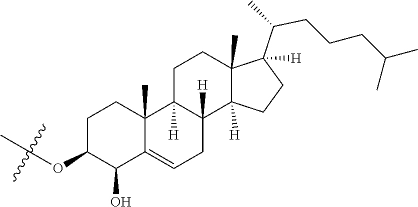 |
| 1778 | 1 | 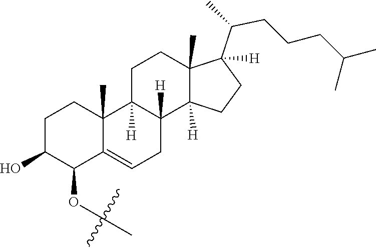 |
| 1779 | 2 | 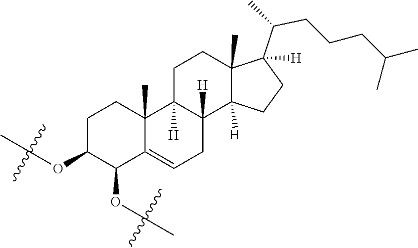 |
| 1780 | 1 | 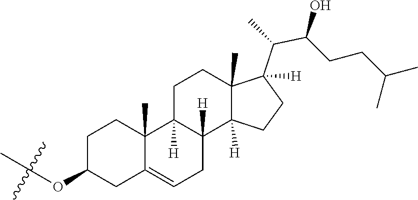 |
| 1781 | 1 | 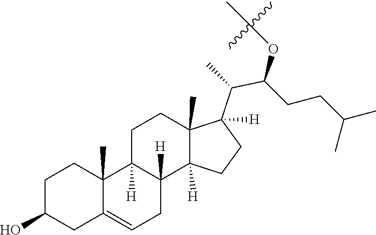 |
| 1782 | 2 | 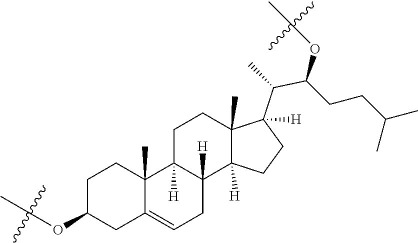 |
| 1783 | 1 | 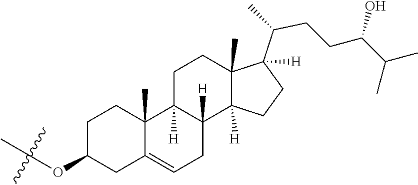 |
| 1784 | 1 | 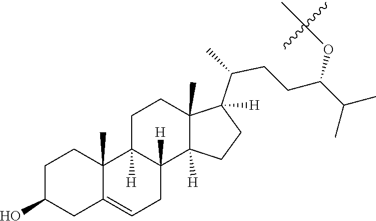 |
| 1785 | 2 | 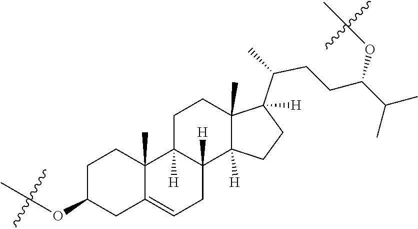 |
| 1786 | 1 | 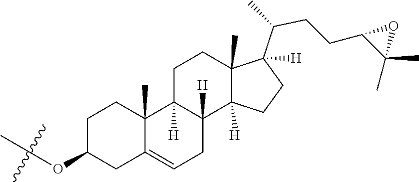 |
| 1787 | 1 | 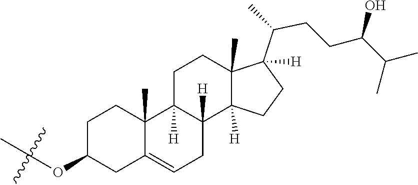 |
| 1788 | 1 | 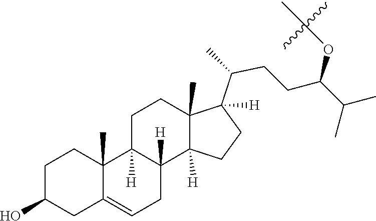 |
| 1789 | 2 | 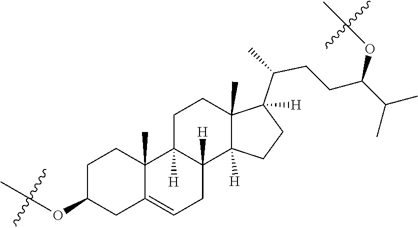 |
| 1790 | 1 | 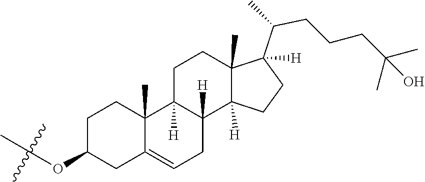 |
| 1791 | 1 | 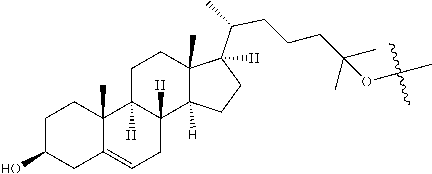 |
| 1792 | 2 | 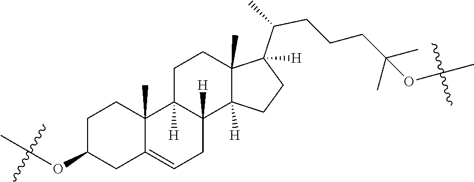 |
| 1793 | 1 | 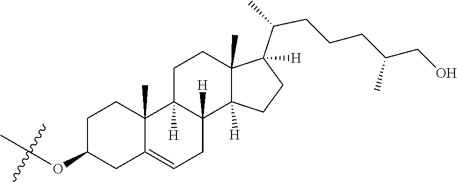 |
| 1794 | 1 | 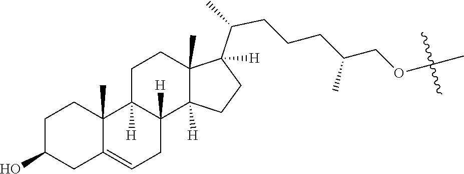 |
| 1795 | 2 | 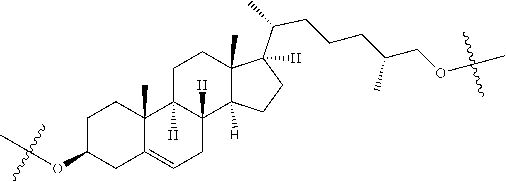 |
| 1796 | 1 | 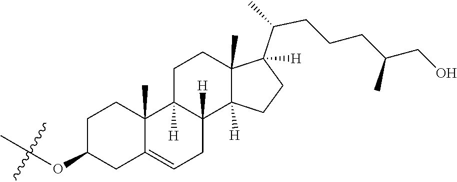 |
| 1797 | 1 | 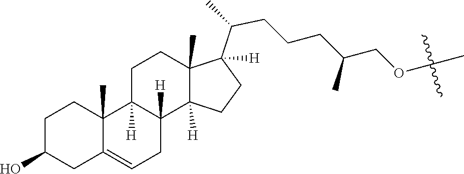 |
| 1798 | 2 | 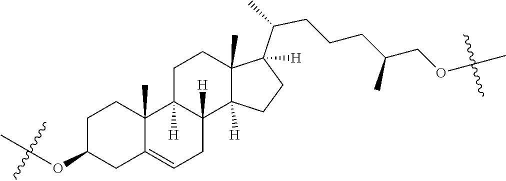 |
| 1799 | 1 | 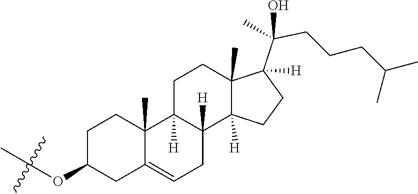 |
| 1800 | 1 | 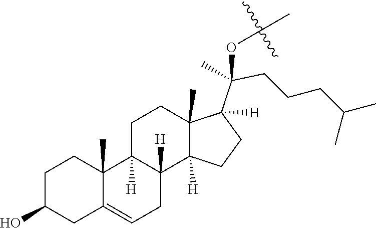 |
| 1801 | 2 | 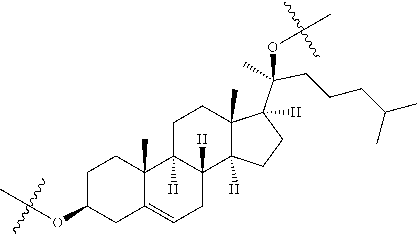 |
| 1802 | 1 | 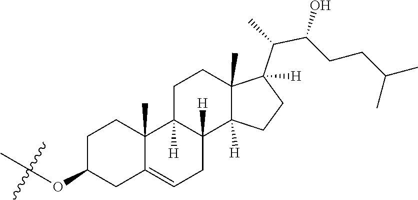 |
| 1803 | 1 | 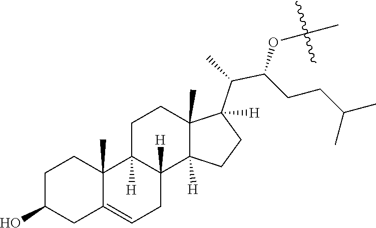 |
| 1804 | 2 | 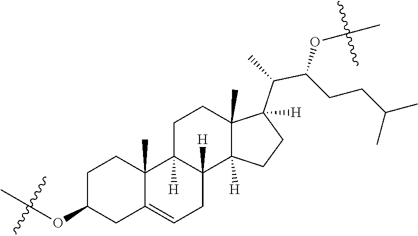 |
| 1805 | 1 | 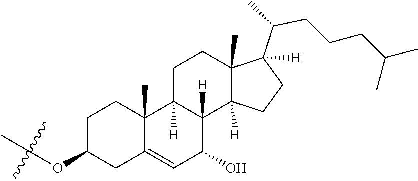 |
| 1806 | 1 | 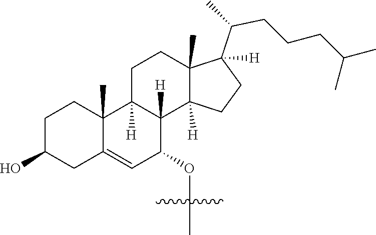 |
| 1807 | 2 | 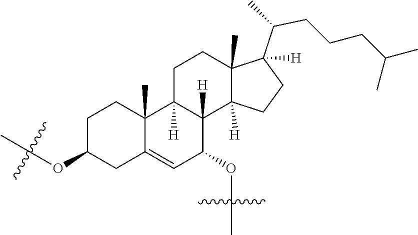 |
| 1808 | 1 | 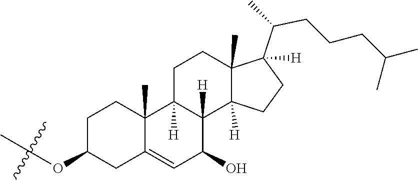 |
| 1809 | 1 | 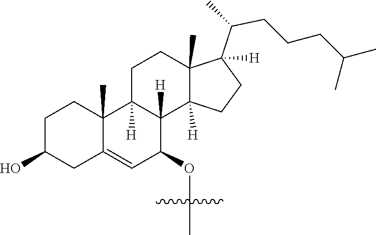 |
| 1810 | 2 | 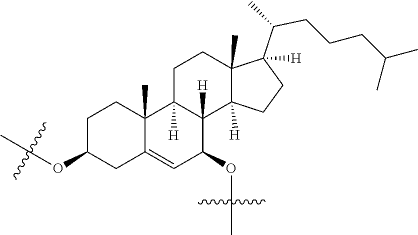 |
| 1811 | 1 | 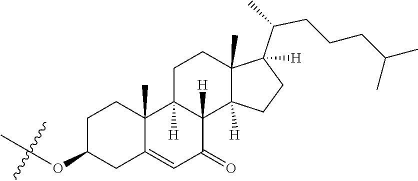 |
| 1812 | 1 | 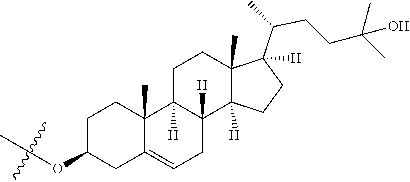 |
| 1813 | 1 | 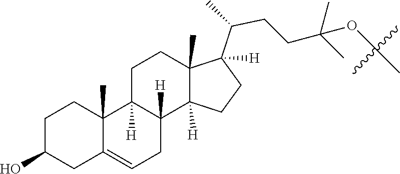 |
| 1814 | 2 | 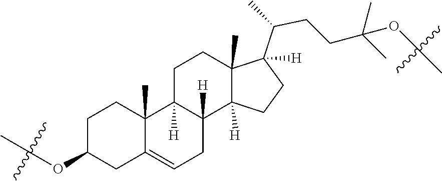 |
| 1815 | 1 | 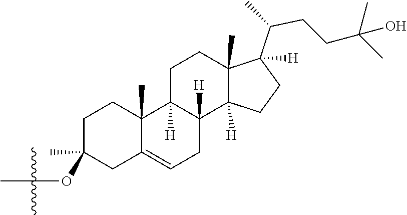 |
| 1816 | 1 | 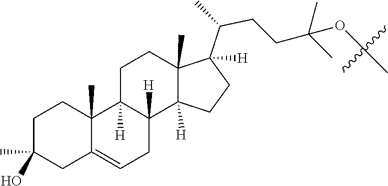 |
| 1817 | 2 | 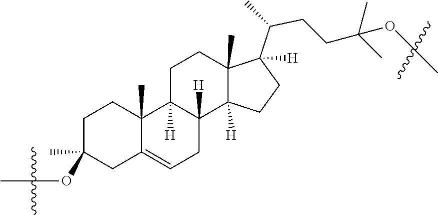 |
| 1818 | 1 | 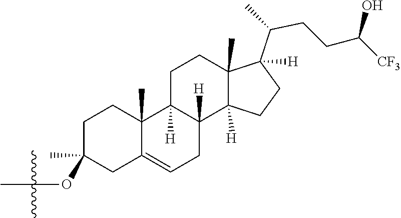 |
| 1819 | 1 | 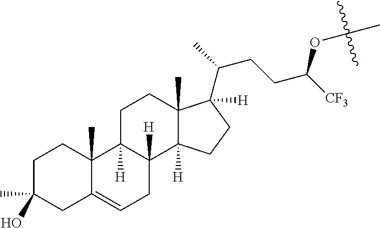 |
| 1820 | 2 | 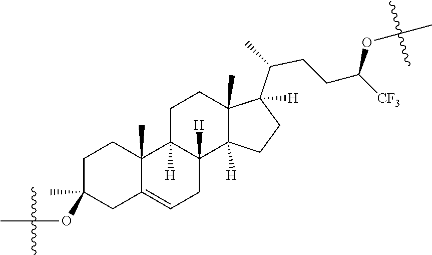 |
| 1821 | 1 | 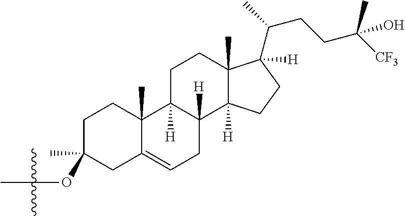 |
| 1822 | 1 | 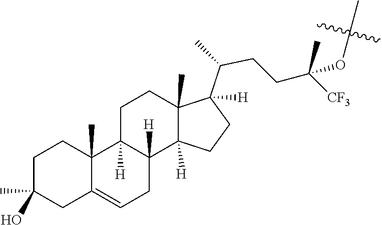 |
| 1823 | 2 | 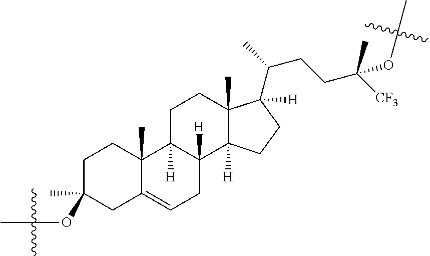 |
| 1824 | 1 | 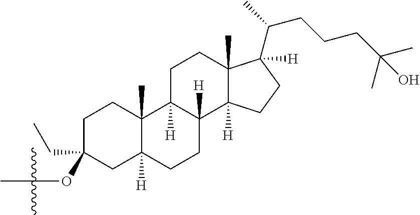 |
| 1825 | 1 | 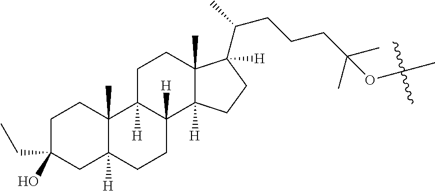 |
| 1826 | 2 | 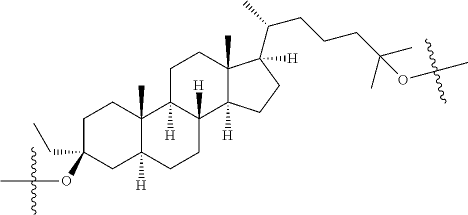 |
| 1827 | 1 | 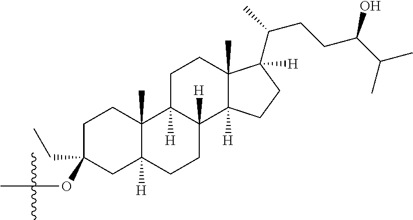 |
| 1828 | 1 | 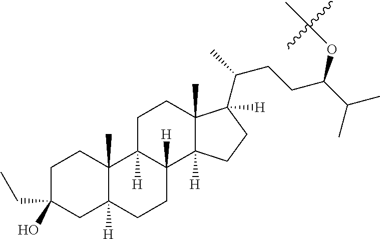 |
| 1829 | 2 | 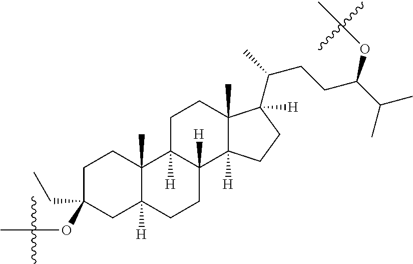 |
| 1830 | 1 | 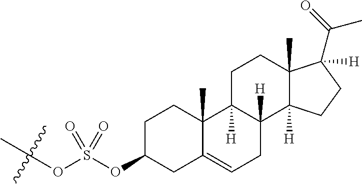 |
| 1831 | 1 | 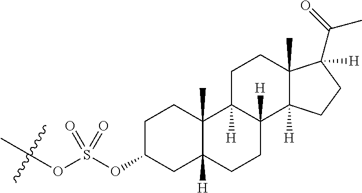 |
| 1832 | 1 | 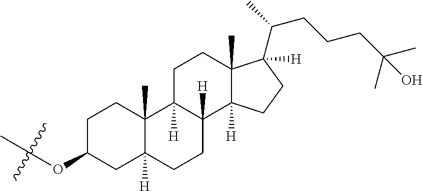 |
| 1833 | 1 | 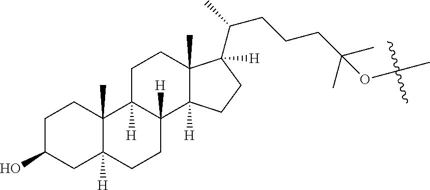 |
| 1834 | 2 | 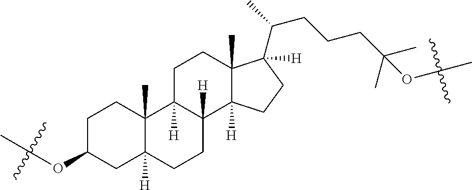 |
| 1835 | 1 | 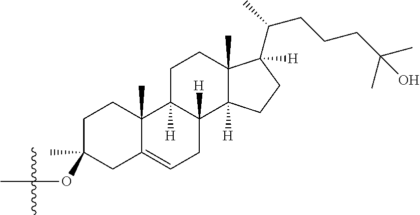 |
| 1836 | 1 | 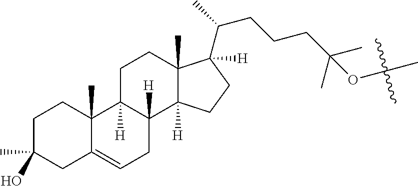 |
| 1837 | 2 | 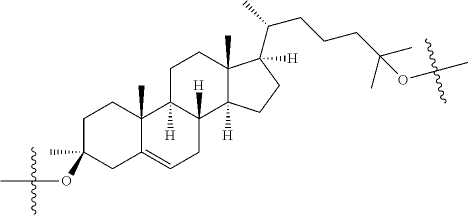 |
| 1838 | 1 | 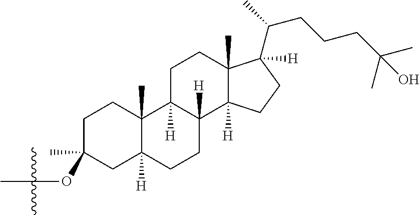 |
| 1839 | 1 | 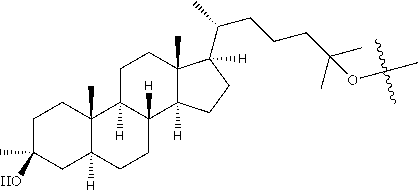 |
| 1840 | 2 | 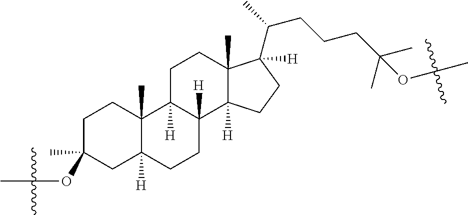 |
| 1841 | 1 | 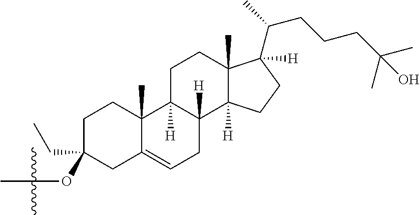 |
| 1842 | 1 | 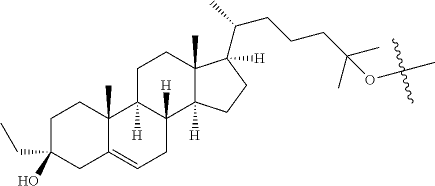 |
| 1843 | 2 | 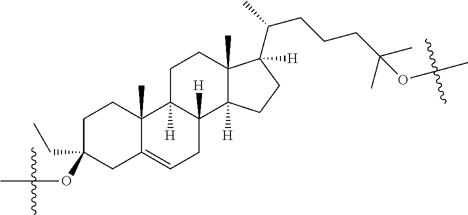 |
| 1844 | 1 | 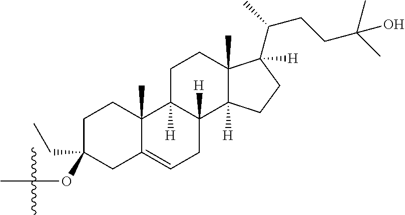 |
| 1845 | 1 | 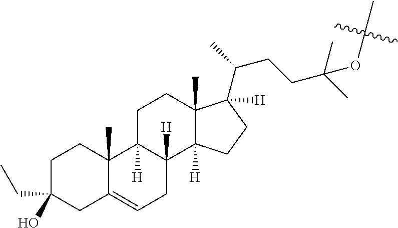 |
| 1846 | 2 | 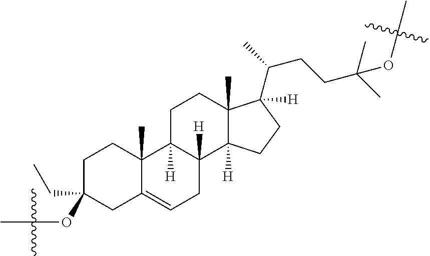 |

In some embodiments, the compound is selected from any of the compounds depicted in in Table 28, pharmaceutically acceptable salts thereof, deuterated variants thereof or combinations thereof.

TABLE 28

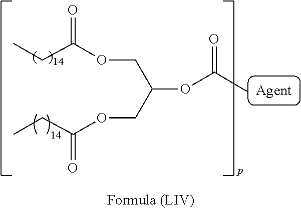

| Cpd. No. | p | 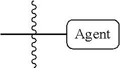 |
|---|---|---|
| 1847 | 1 | 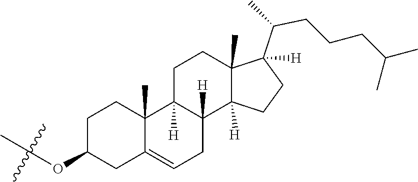 |
| 1848 | 1 | 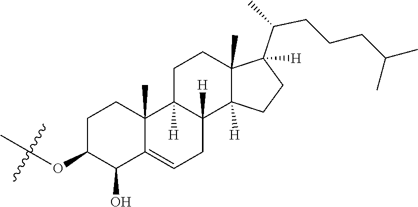 |
| 1849 | 1 | 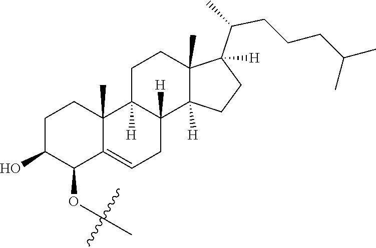 |
| 1850 | 2 | 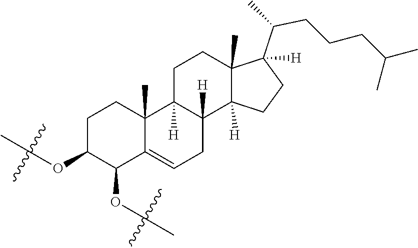 |
| 1851 | 1 | 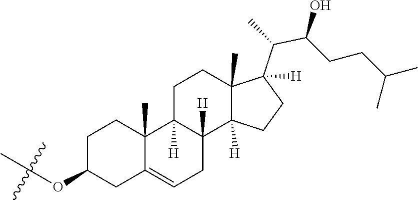 |
| 1852 | 1 | 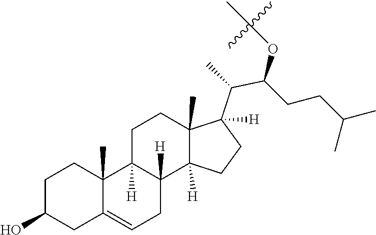 |
| 1853 | 2 | 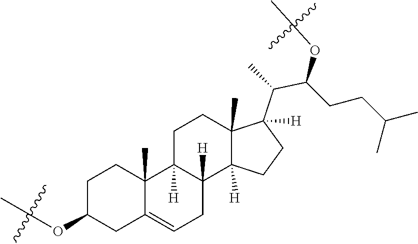 |
| 1854 | 1 | 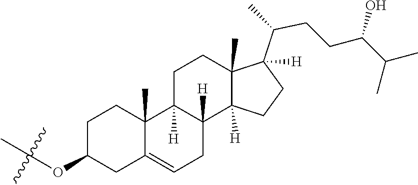 |
| 1855 | 1 | 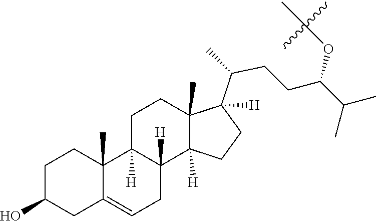 |
| 1856 | 2 | 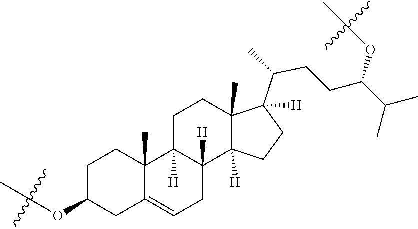 |
| 1857 | 1 | 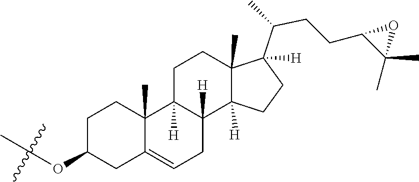 |
| 1858 | 1 | 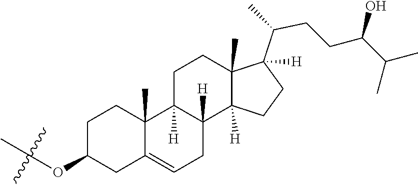 |
| 1859 | 1 | 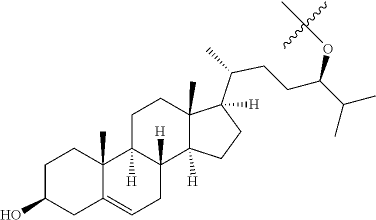 |
| 1860 | 2 | 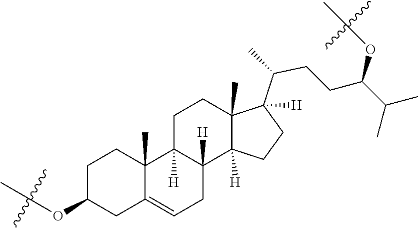 |
| 1861 | 1 | 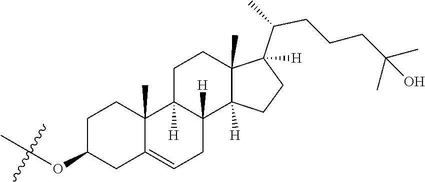 |
| 1862 | 1 | 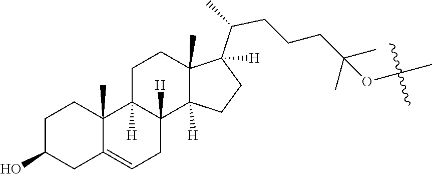 |
| 1863 | 2 | 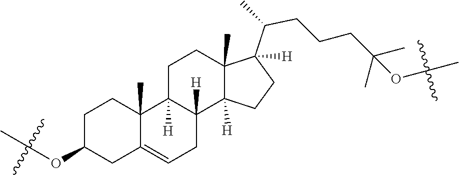 |
| 1864 | 1 | 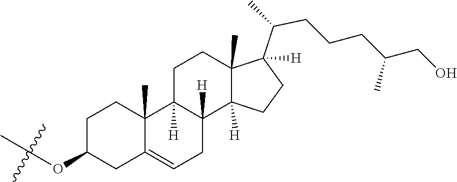 |
| 1865 | 1 | 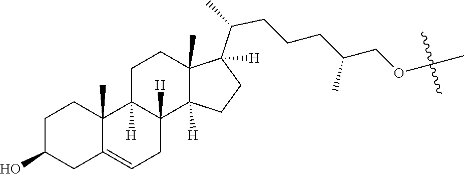 |
| 1866 | 2 | 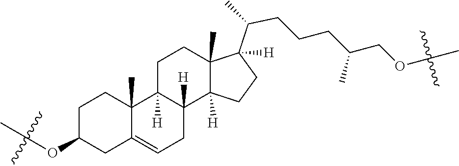 |
| 1867 | 1 | 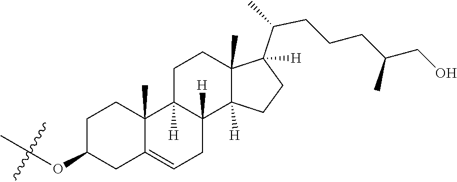 |
| 1868 | 1 | 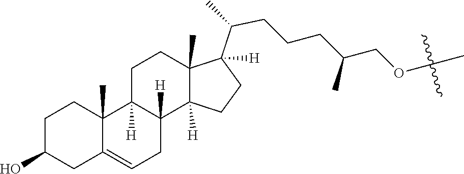 |
| 1869 | 2 | 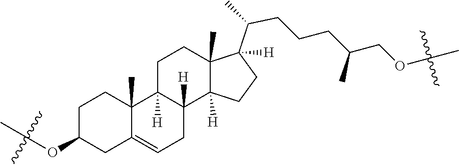 |
| 1870 | 1 | 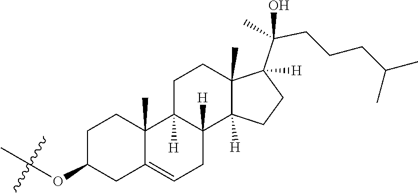 |
| 1871 | 1 | 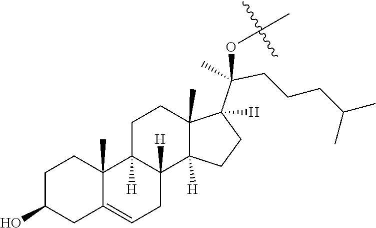 |
| 1872 | 2 | 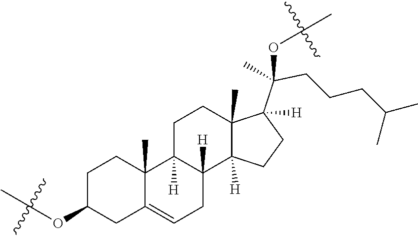 |
| 1873 | 1 | 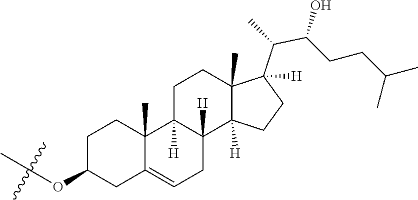 |
| 1874 | 1 | 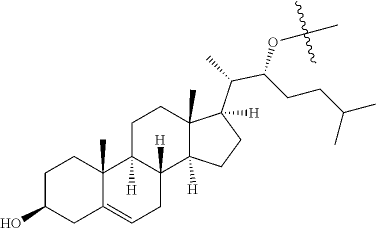 |
| 1875 | 2 | 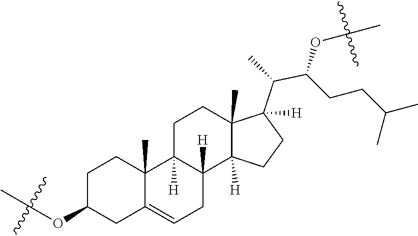 |
| 1876 | 1 | 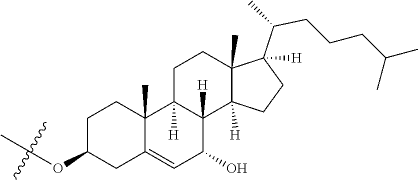 |
| 1877 | 1 | 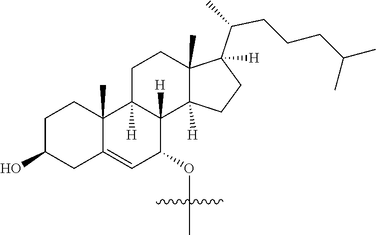 |
| 1878 | 2 | 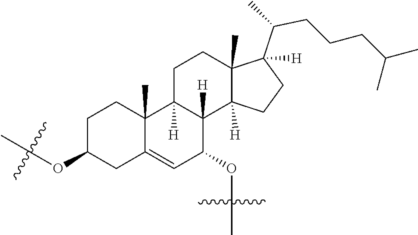 |
| 1879 | 1 | 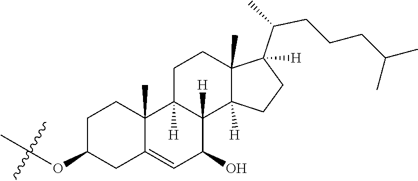 |
| 1880 | 1 | 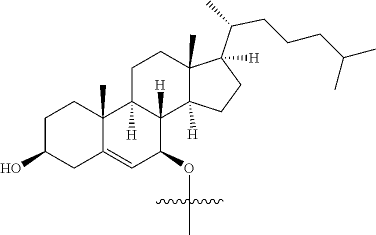 |
| 1881 | 2 | 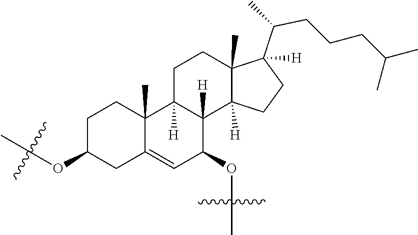 |
| 1882 | 1 | 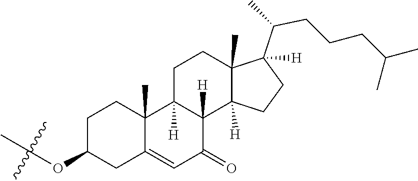 |
| 1883 | 1 | 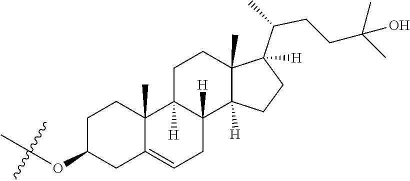 |
| 1884 | 1 | 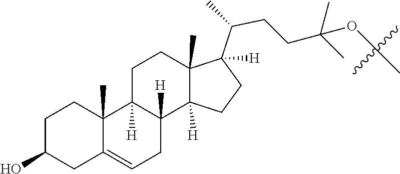 |
| 1885 | 2 | 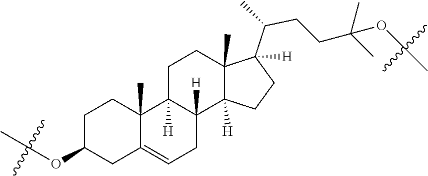 |
| 1886 | 1 | 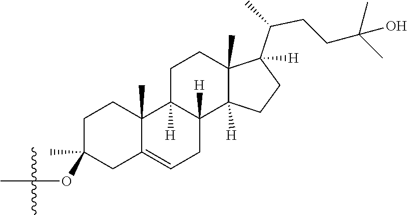 |
| 1887 | 1 | 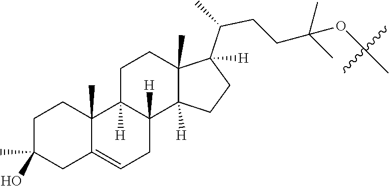 |
| 1888 | 2 | 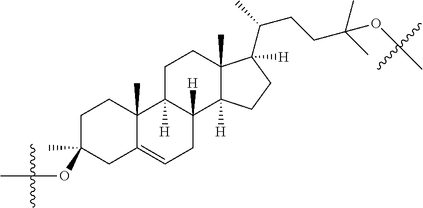 |
| 1889 | 1 | 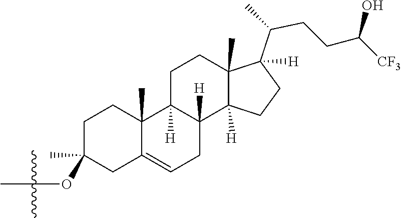 |
| 1890 | 1 | 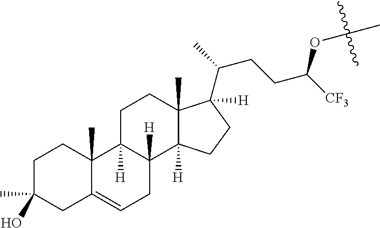 |
| 1891 | 2 | 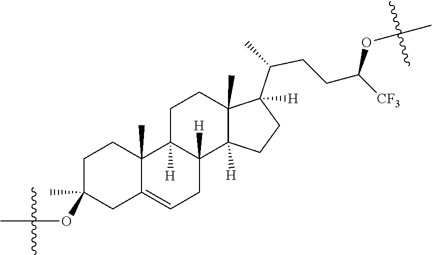 |
| 1892 | 1 | 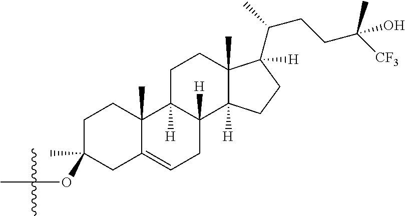 |
| 1893 | 1 | 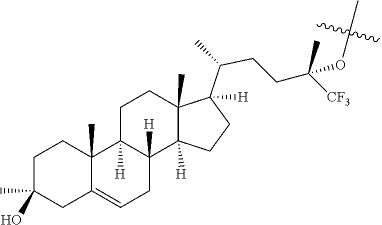 |
| 1894 | 2 | 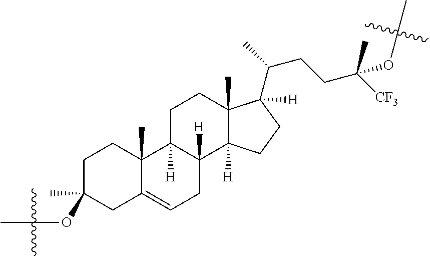 |
| 1895 | 1 | 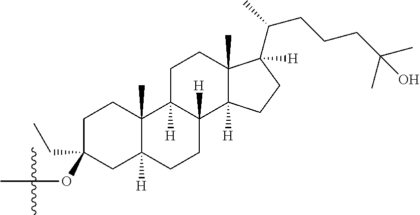 |
| 1896 | 1 | 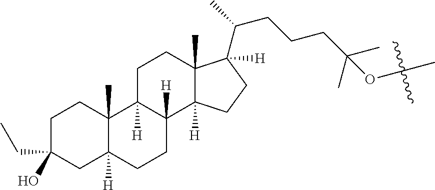 |
| 1897 | 2 | 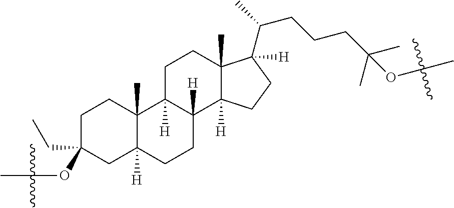 |
| 1898 | 1 | 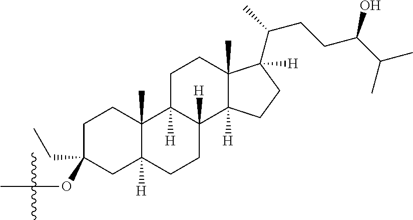 |
| 1899 | 1 | 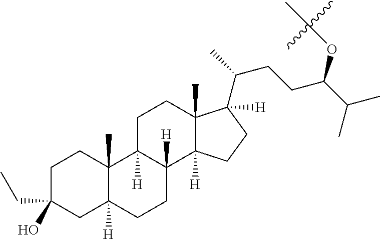 |
| 1900 | 2 | 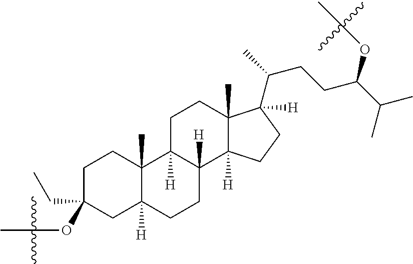 |
| 1901 | 1 | 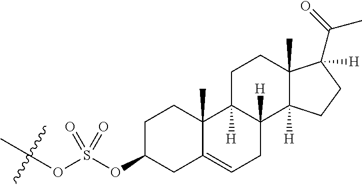 |
| 1902 | 1 | 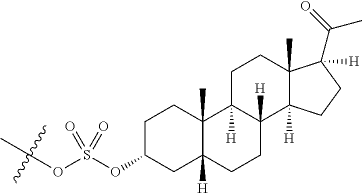 |
| 1903 | 1 | 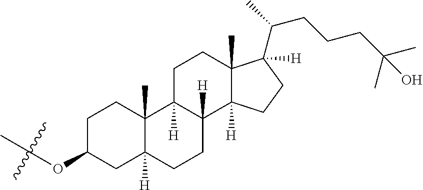 |
| 1904 | 1 | 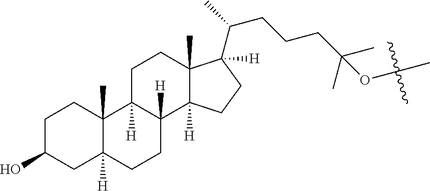 |
| 1905 | 2 | 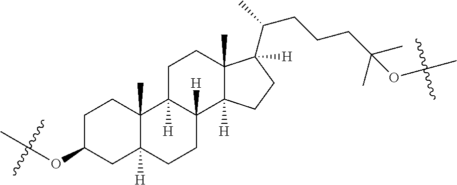 |
| 1906 | 1 | 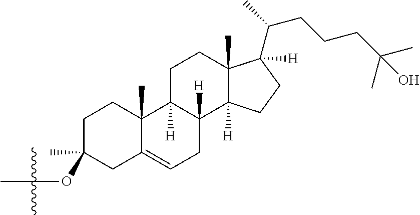 |
| 1907 | 1 | 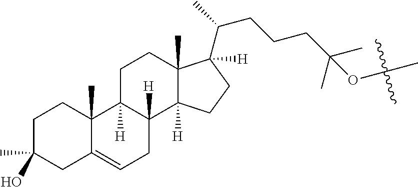 |
| 1908 | 2 | 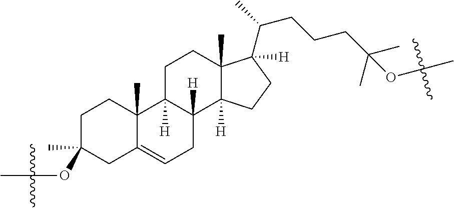 |
| 1909 | 1 | 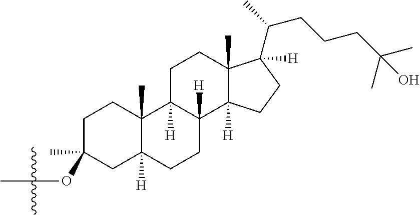 |
| 1910 | 1 | 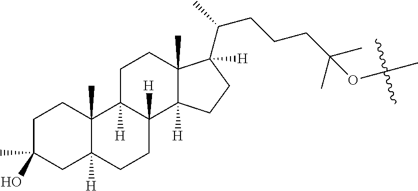 |
| 1911 | 2 | 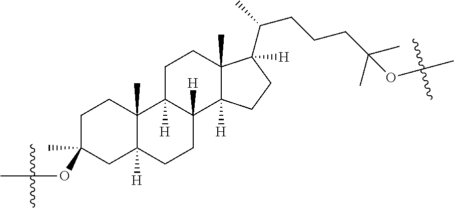 |
| 1912 | 1 | 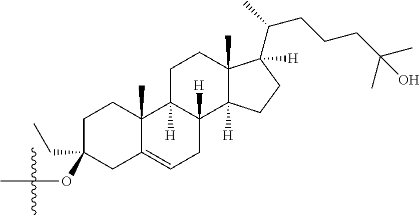 |
| 1913 | 1 | 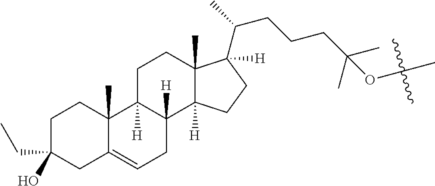 |
| 1914 | 2 | 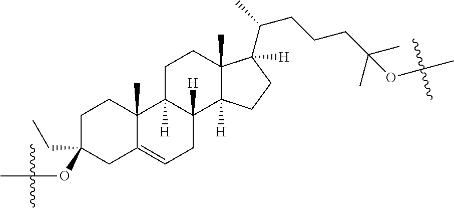 |
| 1915 | 1 | 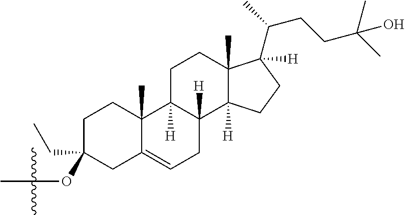 |
| 1916 | 1 | 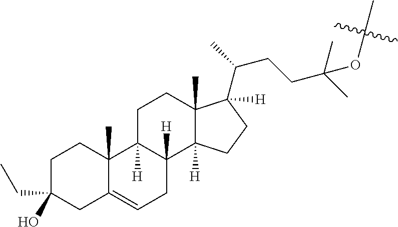 |
| 1917 | 2 | 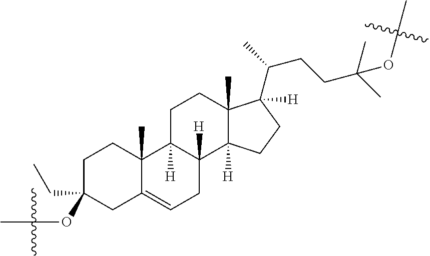 |

In some embodiments, the compound is selected from any of the compounds depicted in in Table 29, pharmaceutically acceptable salts thereof, deuterated variants thereof or combinations thereof.

TABLE 29

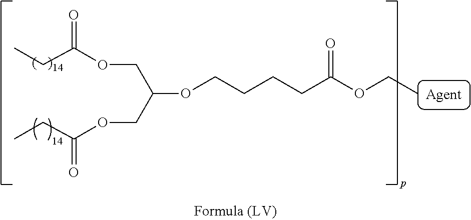

| Cpd. No. | p | 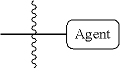 |
|---|---|---|
| 1918 | 1 | 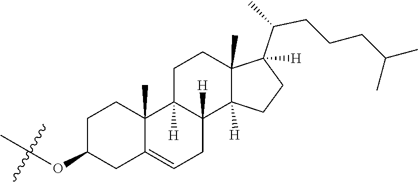 |
| 1919 | 1 | 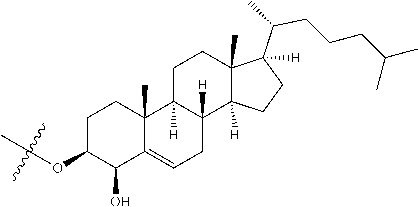 |
| 1920 | 1 | 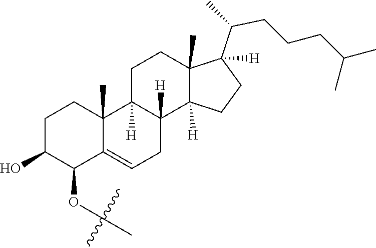 |
| 1921 | 2 | 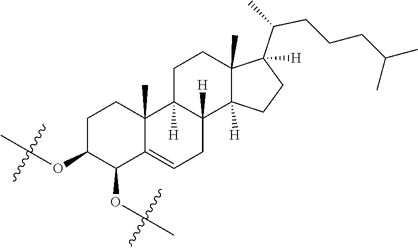 |
| 1922 | 1 | 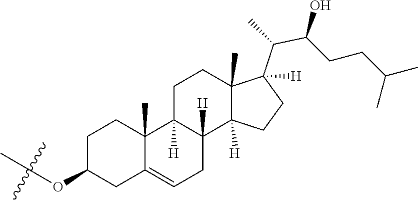 |
| 1923 | 1 | 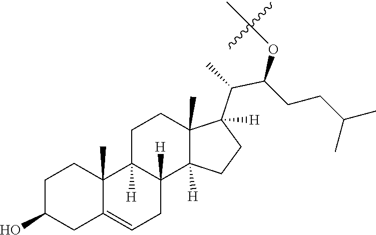 |
| 1924 | 2 | 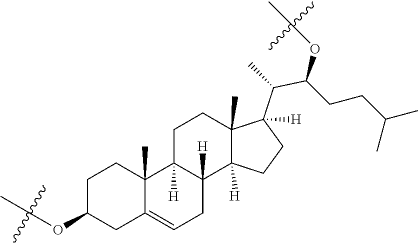 |
| 1925 | 1 | 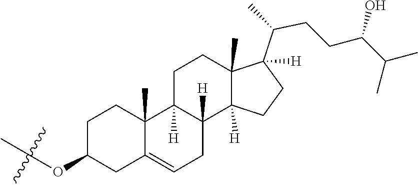 |
| 1926 | 1 | 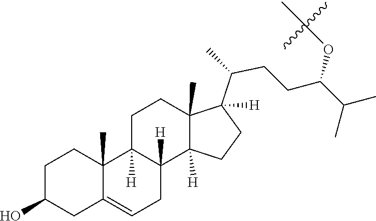 |
| 1927 | 2 | 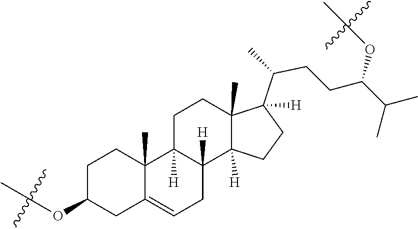 |
| 1928 | 1 | 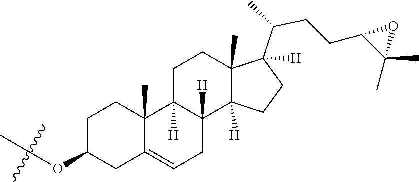 |
| 1929 | 1 | 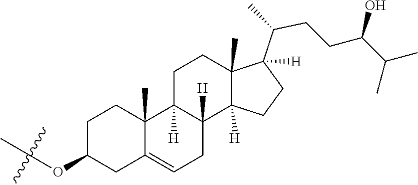 |
| 1930 | 1 | 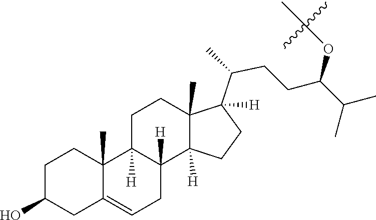 |
| 1931 | 2 | 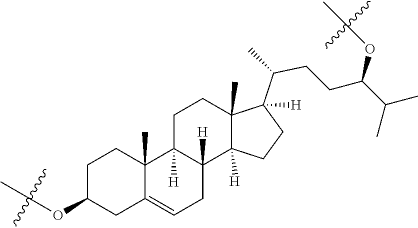 |
| 1932 | 1 | 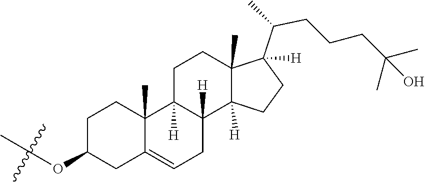 |
| 1933 | 1 | 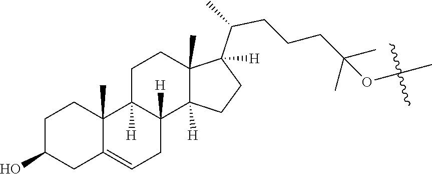 |
| 1934 | 2 | 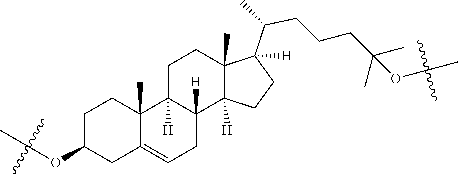 |
| 1935 | 1 | 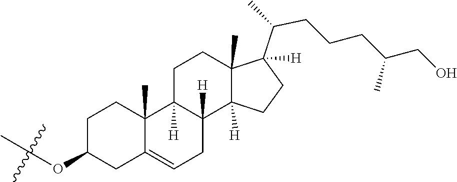 |
| 1936 | 1 | 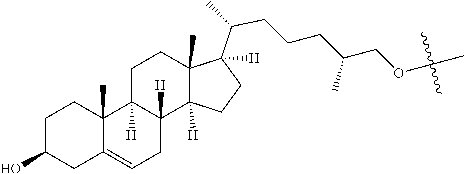 |
| 1937 | 2 | 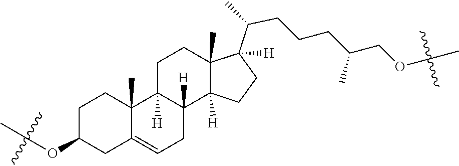 |
| 1938 | 1 | 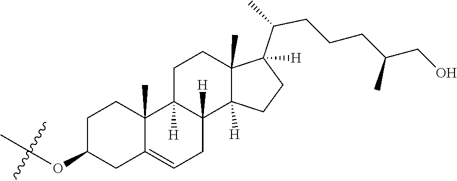 |
| 1939 | 1 | 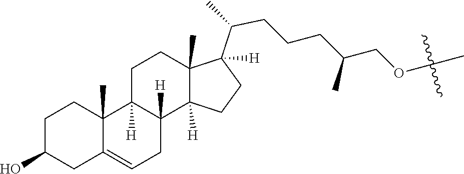 |
| 1940 | 2 | 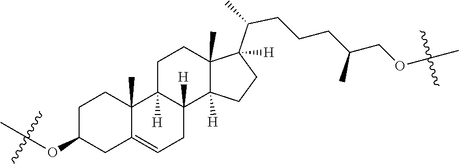 |
| 1941 | 1 | 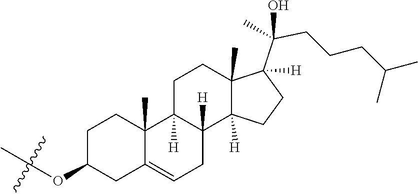 |
| 1942 | 1 | 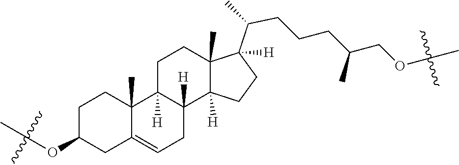 |
| 1943 | 2 | 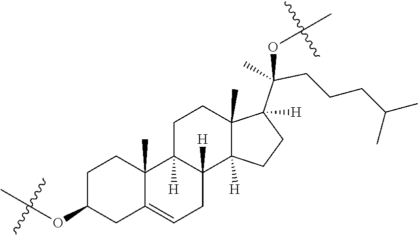 |
| 1944 | 1 | 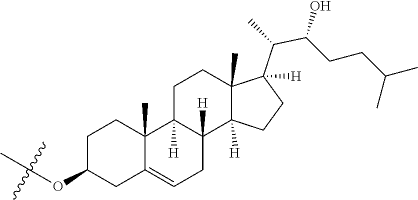 |
| 1945 | 1 | 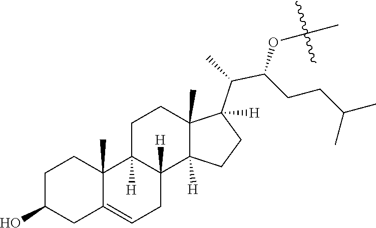 |
| 1946 | 2 | 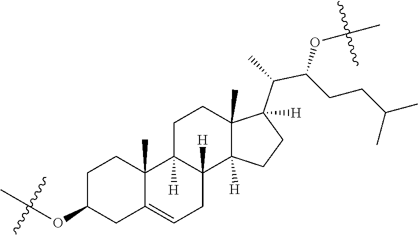 |
| 1947 | 1 | 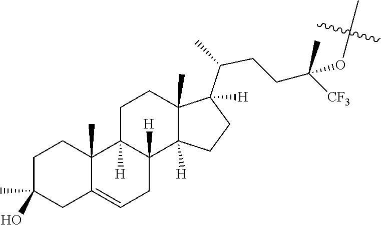 |
| 1948 | 1 | 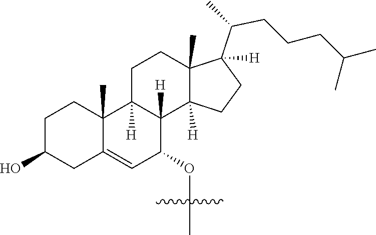 |
| 1949 | 2 | 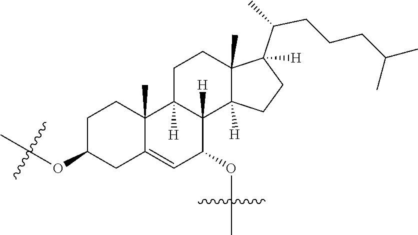 |
| 1950 | 1 | 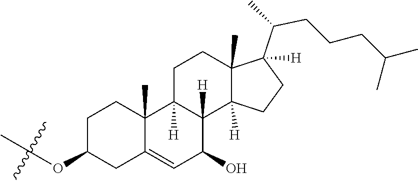 |
| 1951 | 1 | 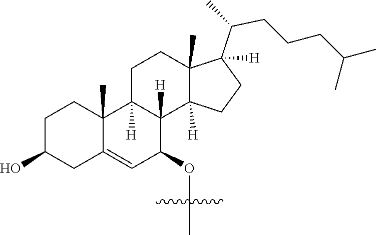 |
| 1952 | 2 | 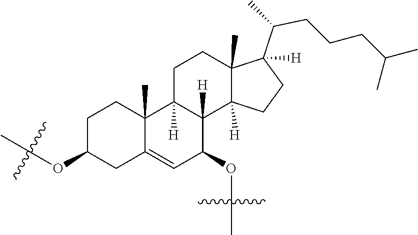 |
| 1953 | 1 | 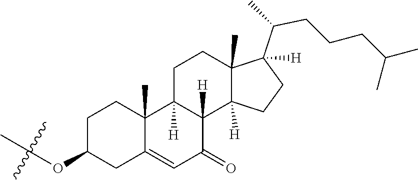 |
| 1954 | 1 | 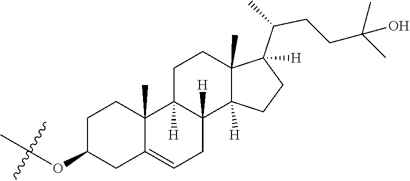 |
| 1955 | 1 | 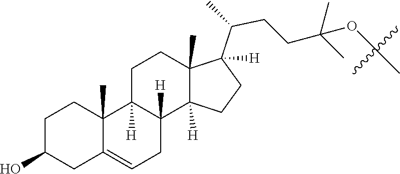 |
| 1956 | 2 | 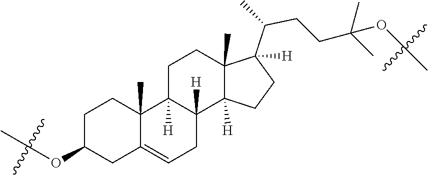 |
| 1957 | 1 | 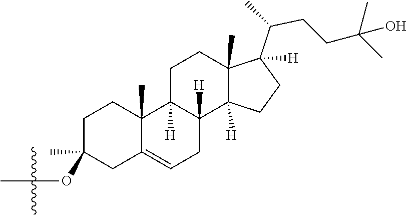 |
| 1958 | 1 | 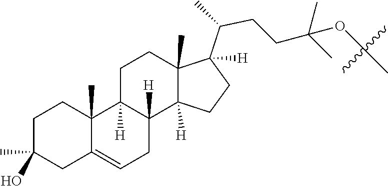 |
| 1959 | 2 | 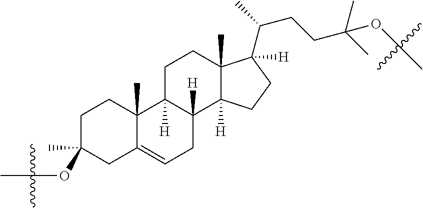 |
| 1960 | 1 | 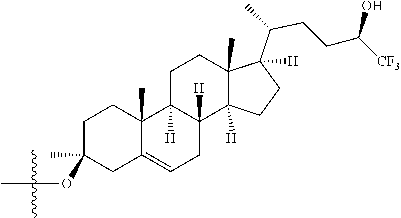 |
| 1961 | 1 | 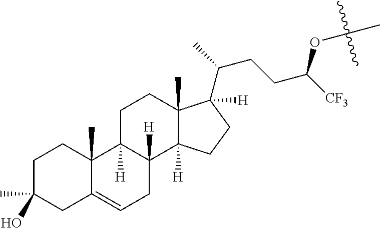 |
| 1962 | 2 | 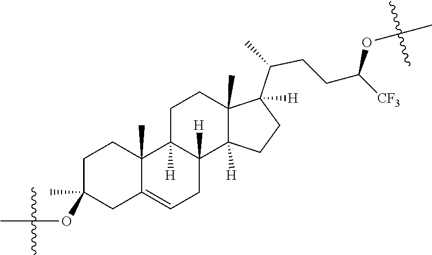 |
| 1963 | 1 | 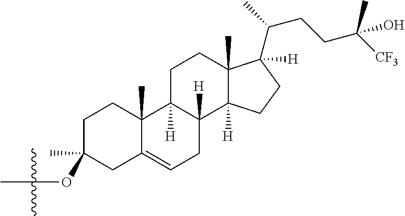 |
| 1964 | 1 | 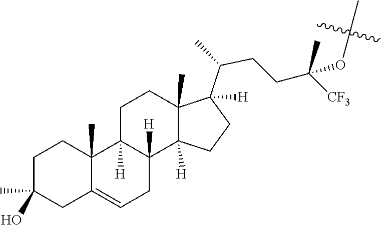 |
| 1965 | 2 | 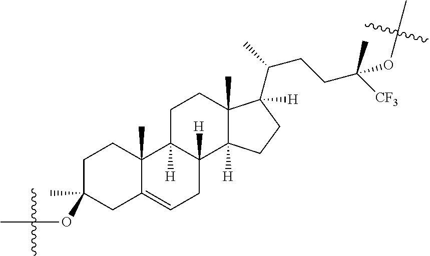 |
| 1966 | 1 | 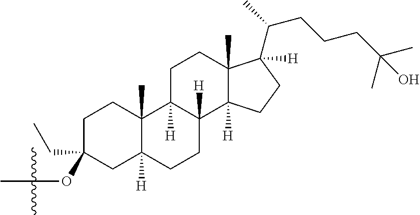 |
| 1967 | 1 | 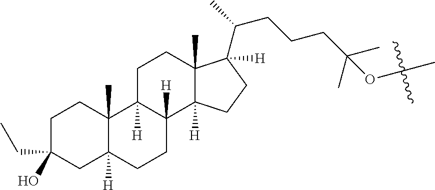 |
| 1968 | 2 | 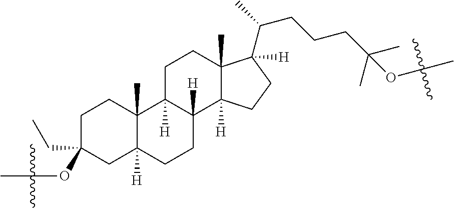 |
| 1969 | 1 | 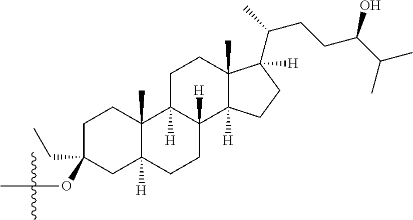 |
| 1970 | 1 | 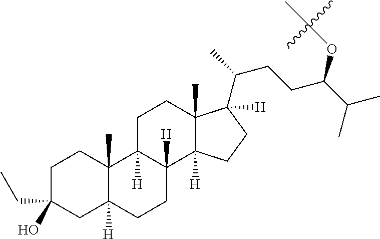 |
| 1971 | 2 | 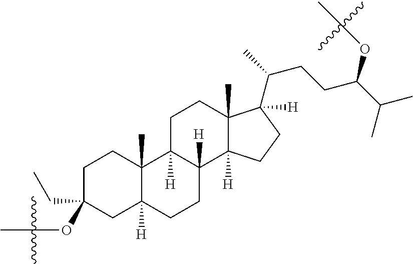 |
| 1972 | 1 | 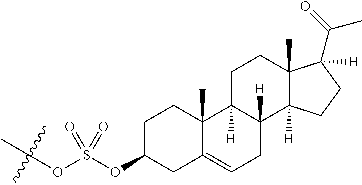 |
| 1973 | 1 | 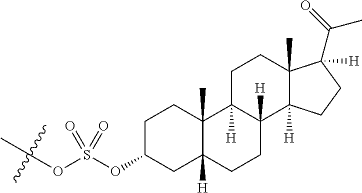 |
| 1974 | 1 | 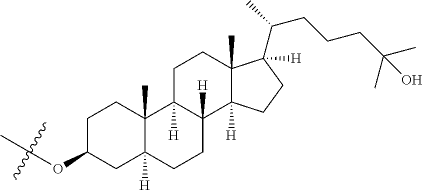 |
| 1975 | 1 | 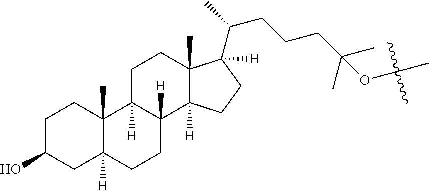 |
| 1976 | 2 | 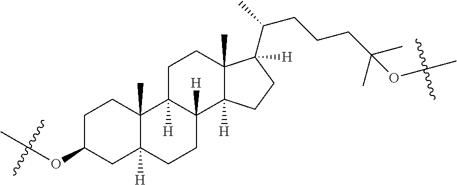 |
| 1977 | 1 | 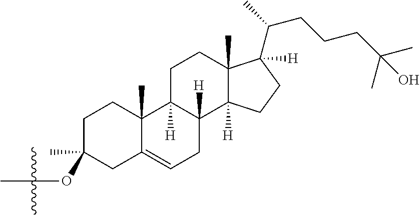 |
| 1978 | 1 | 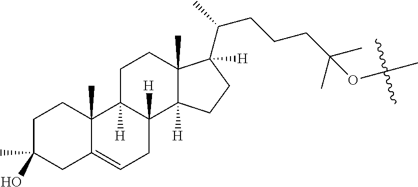 |
| 1979 | 2 | 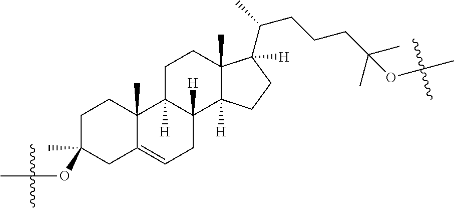 |
| 1980 | 1 | 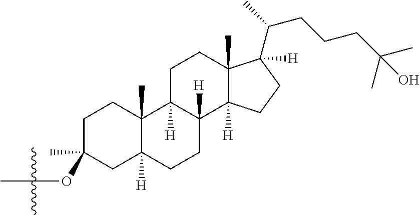 |
| 1981 | 1 | 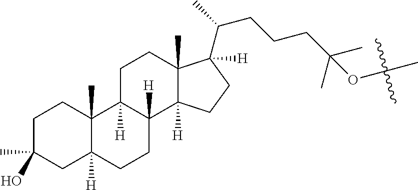 |
| 1982 | 2 | 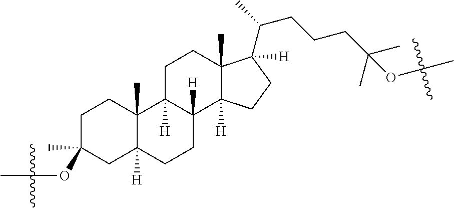 |
| 1983 | 1 | 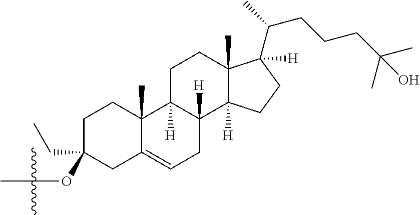 |
| 1984 | 1 | 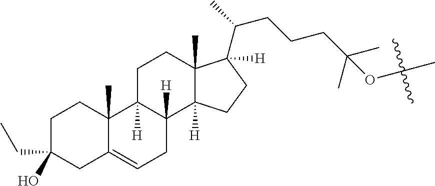 |
| 1985 | 2 | 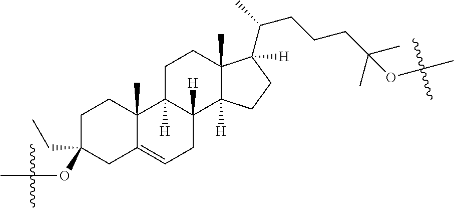 |
| 1986 | 1 | 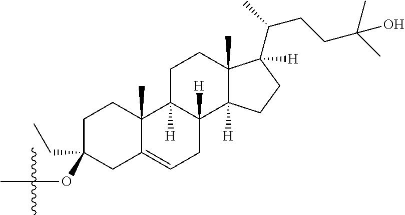 |
| 1987 | 1 | 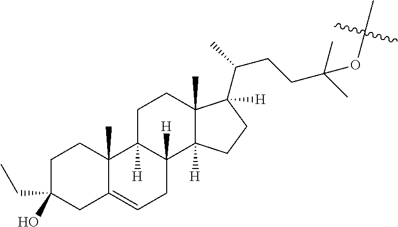 |
| 1988 | 2 | 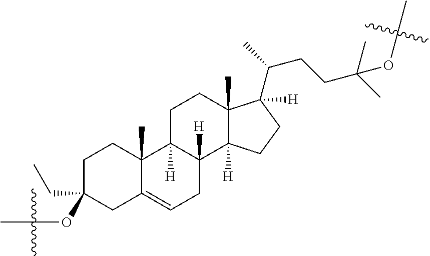 |

In some embodiments, the compound is selected from any of the compounds depicted in in Table 30, pharmaceutically acceptable salts thereof, deuterated variants thereof or combinations thereof.

TABLE 30

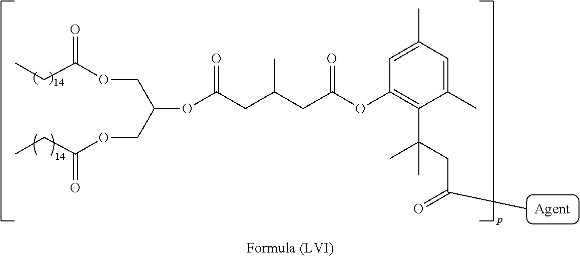

| Cpd. No. | p | 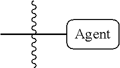 |
|---|---|---|
| 1989 | 1 | 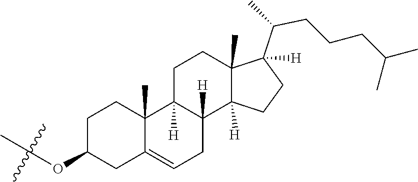 |
| 1990 | 1 | 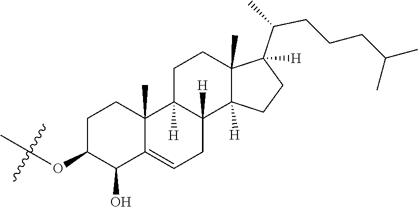 |
| 1991 | 1 | 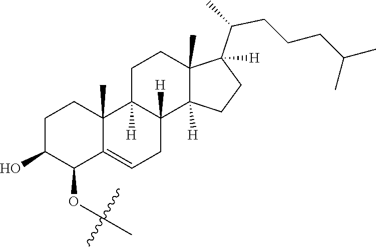 |
| 1992 | 2 | 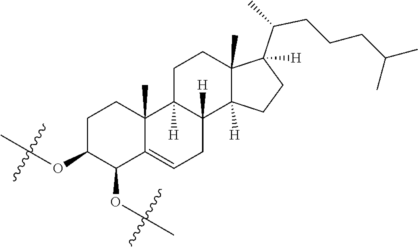 |
| 1993 | 1 | 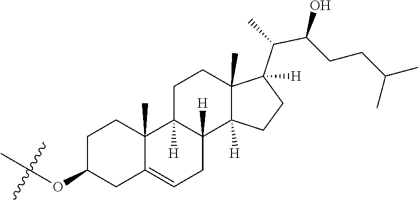 |
| 1994 | 1 | 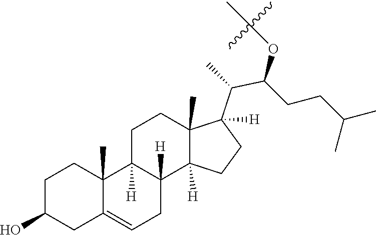 |
| 1995 | 2 | 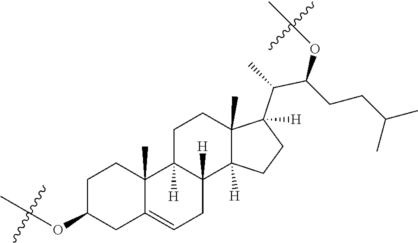 |
| 1996 | 1 | 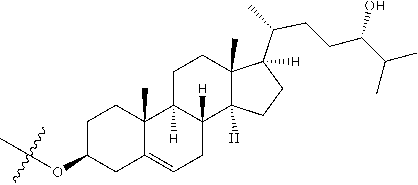 |
| 1997 | 1 | 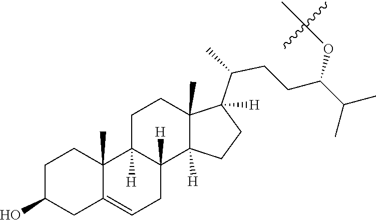 |
| 1998 | 2 | 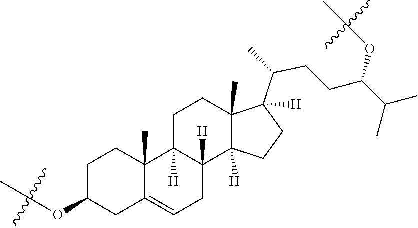 |
| 1999 | 1 | 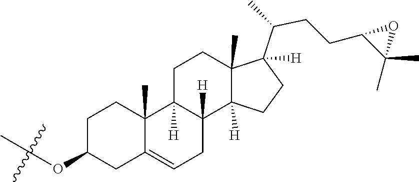 |
| 2000 | 1 | 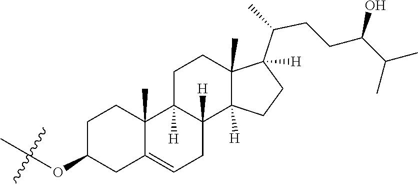 |
| 2001 | 1 | 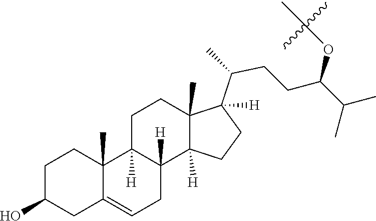 |
| 2002 | 2 | 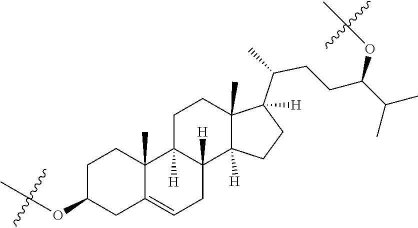 |
| 2003 | 1 | 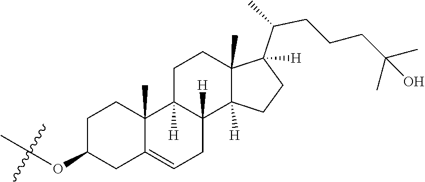 |
| 2004 | 1 | 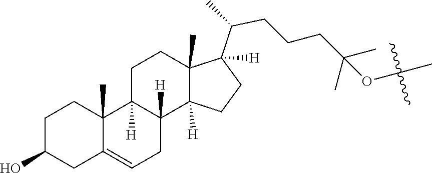 |
| 2005 | 2 | 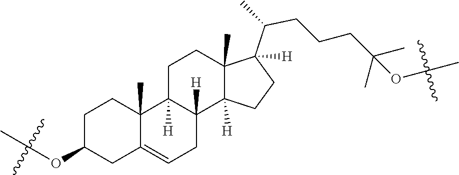 |
| 2006 | 1 | 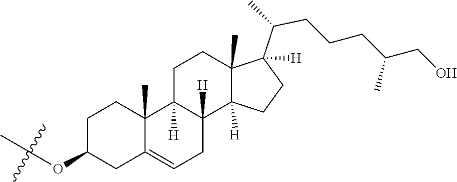 |
| 2007 | 1 | 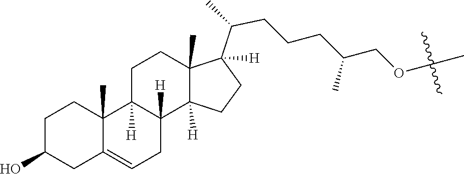 |
| 2008 | 2 | 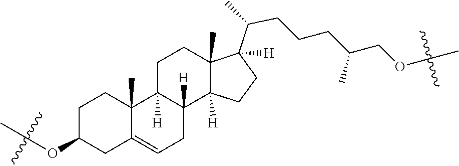 |
| 2009 | 1 | 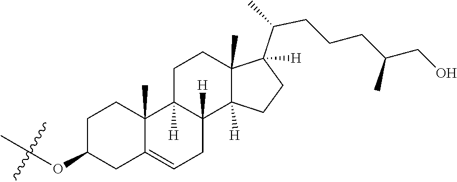 |
| 2010 | 1 | 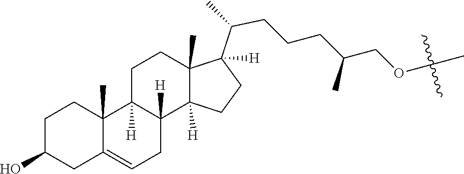 |
| 2011 | 2 | 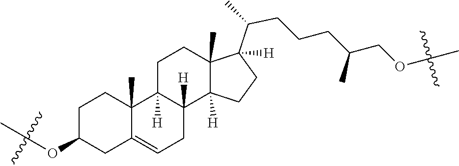 |
| 2012 | 1 | 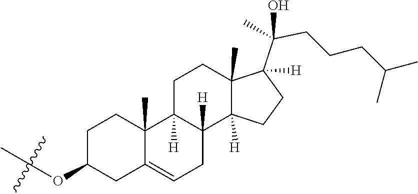 |
| 2013 | 1 | 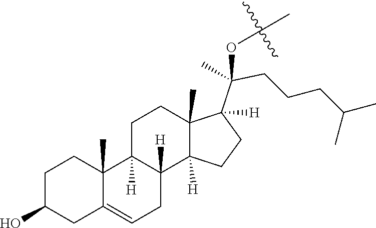 |
| 2014 | 2 | 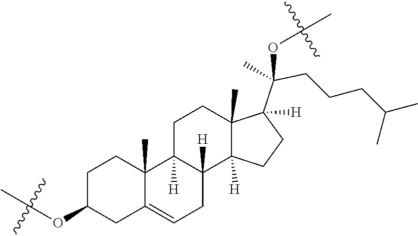 |
| 2015 | 1 | 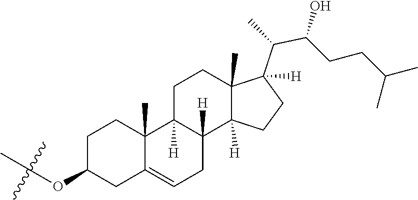 |
| 2016 | 1 | 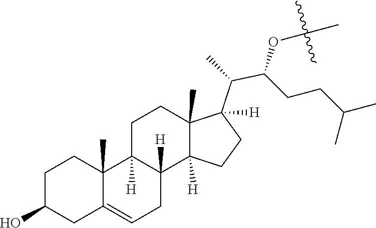 |
| 2017 | 2 | 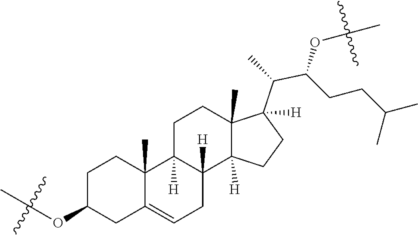 |
| 2018 | 1 | 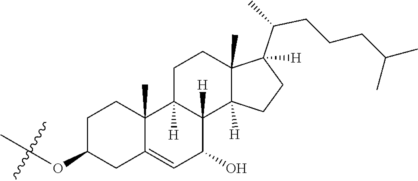 |
| 2019 | 1 | 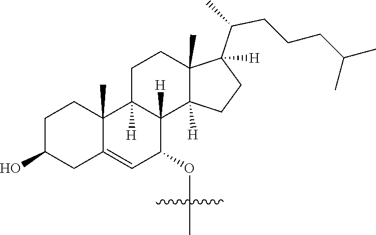 |
| 2020 | 2 | 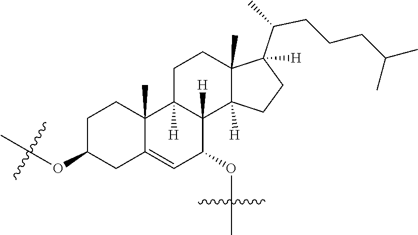 |
| 2021 | 1 | 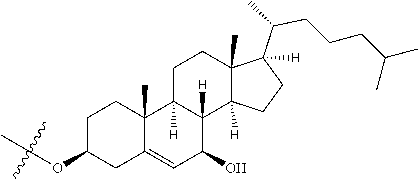 |
| 2022 | 1 | 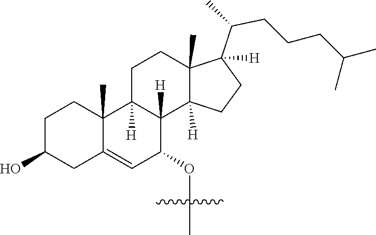 |
| 2023 | 2 | 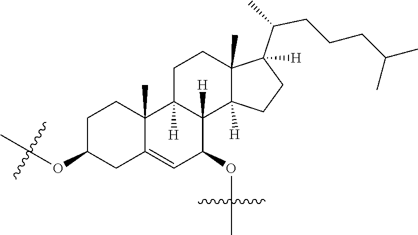 |
| 2024 | 1 | 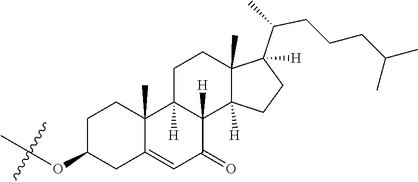 |
| 2025 | 1 | 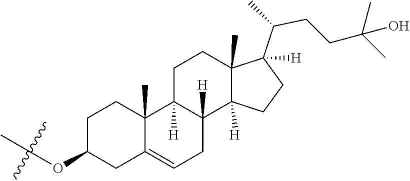 |
| 2026 | 1 | 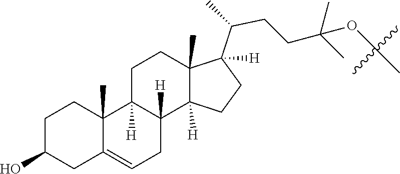 |
| 2027 | 2 | 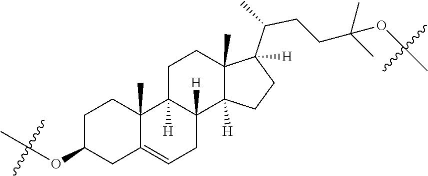 |
| 2028 | 1 | 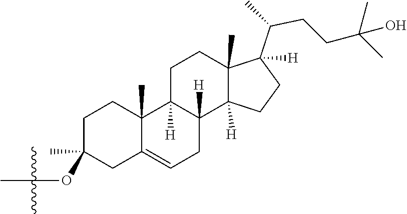 |
| 2029 | 1 | 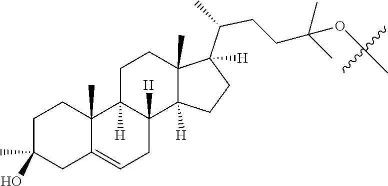 |
| 2030 | 2 | 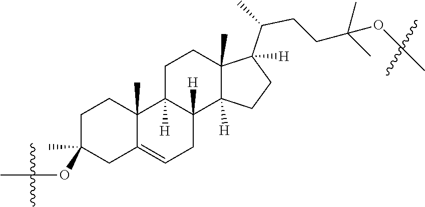 |
| 2031 | 1 | 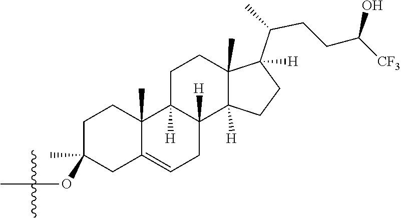 |
| 2032 | 1 | 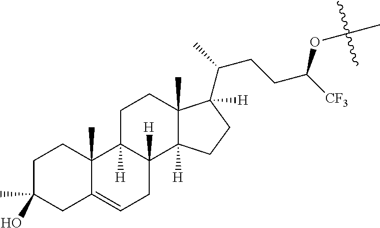 |
| 2033 | 2 | 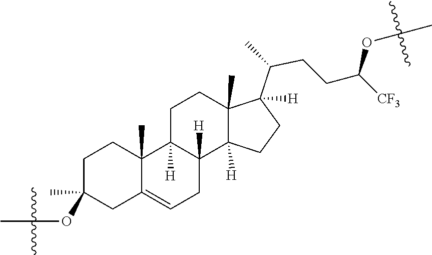 |
| 2034 | 1 | 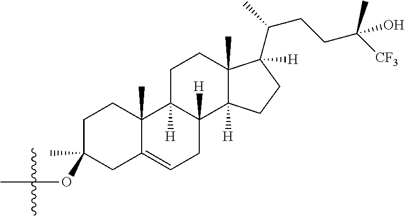 |
| 2035 | 1 | 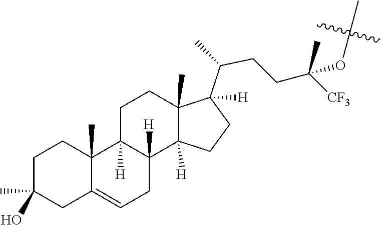 |
| 2036 | 2 | 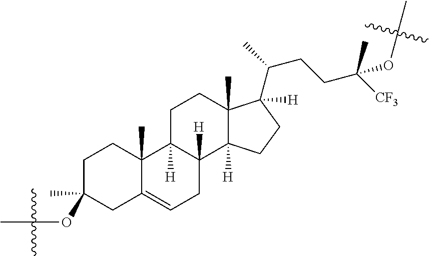 |
| 2037 | 1 | 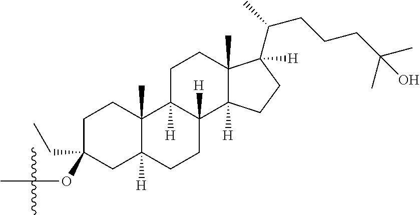 |
| 2038 | 1 | 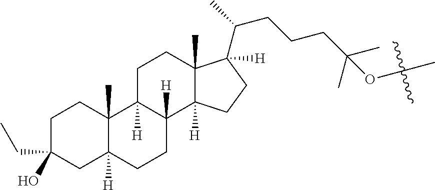 |
| 2039 | 2 | 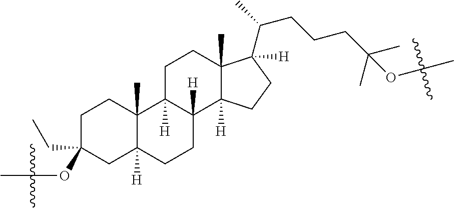 |
| 2040 | 1 | 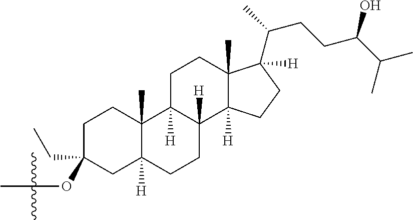 |
| 2041 | 1 | 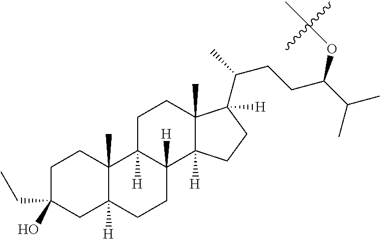 |
| 2042 | 2 | 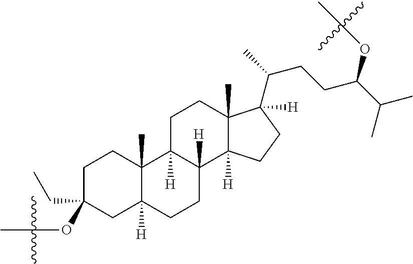 |
| 2043 | 1 | 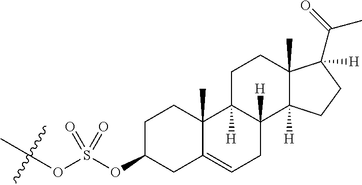 |
| 2044 | 1 | 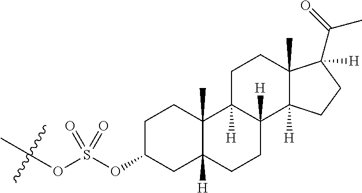 |
| 2045 | 1 | 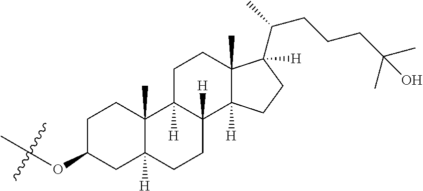 |
| 2046 | 1 | 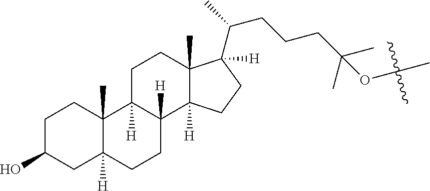 |
| 2047 | 2 | 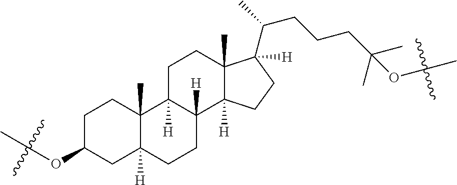 |
| 2048 | 1 | 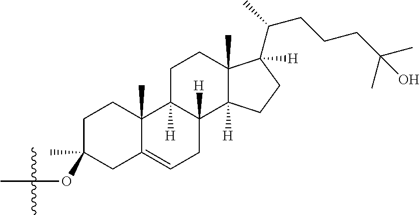 |
| 2049 | 1 | 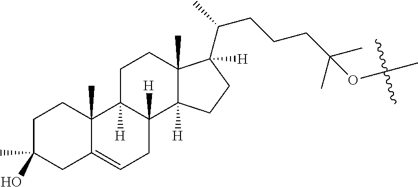 |
| 2050 | 2 | 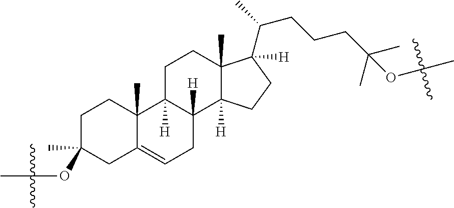 |
| 2051 | 1 | 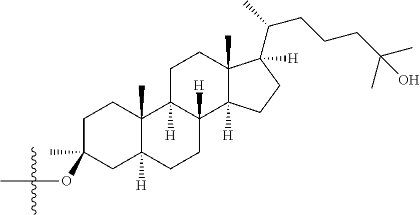 |
| 2052 | 1 | 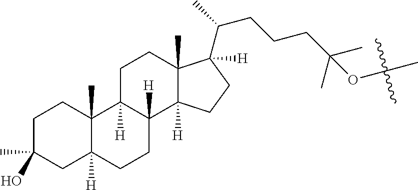 |
| 2053 | 2 | 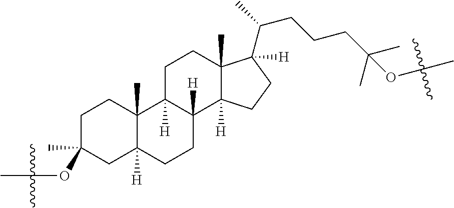 |
| 2054 | 1 | 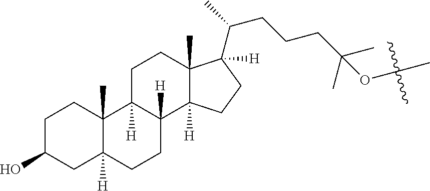 |
| 2055 | 1 | 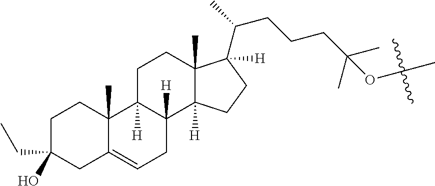 |
| 2056 | 2 | 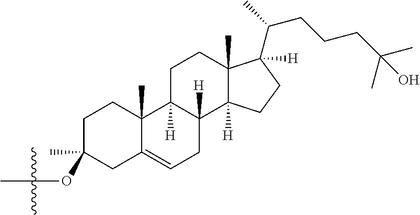 |
| 2057 | 1 | 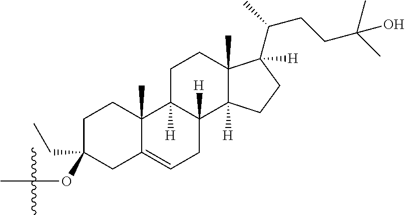 |
| 2058 | 1 | 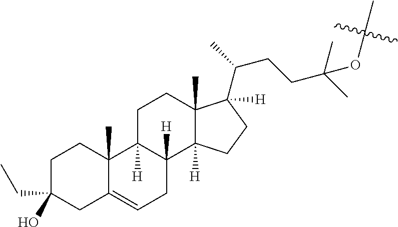 |
| 2059 | 2 | 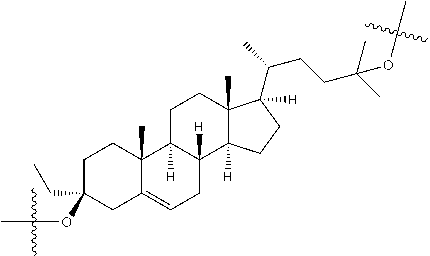 |

In some embodiments, the compound is selected from any of the compounds depicted in in Table 31, pharmaceutically acceptable salts thereof, deuterated variants thereof or combinations thereof.

TABLE 31

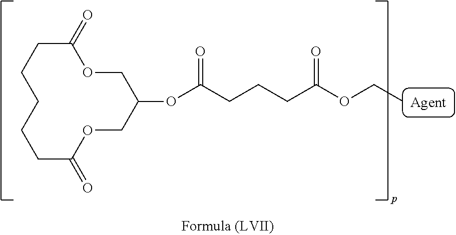

| Cpd. No. | p | 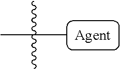 |
|---|---|---|
| 2060 | 1 | 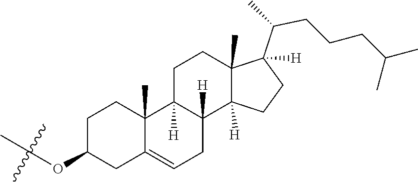 |
| 2061 | 1 | 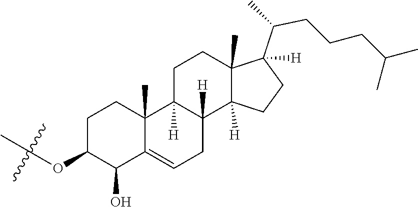 |
| 2062 | 1 | 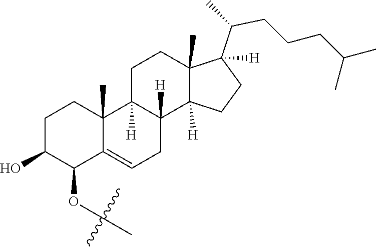 |
| 2063 | 2 | 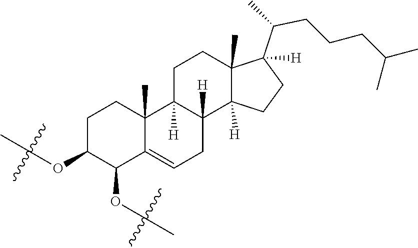 |
| 2064 | 1 | 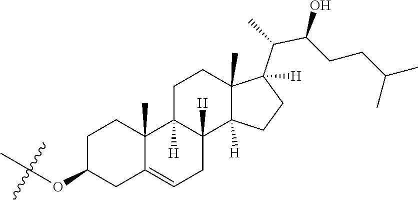 |
| 2065 | 1 | 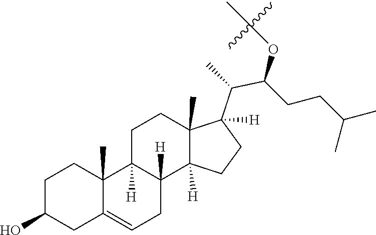 |
| 2066 | 2 | 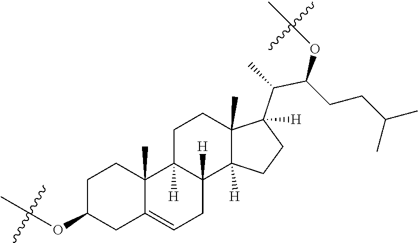 |
| 2067 | 1 | 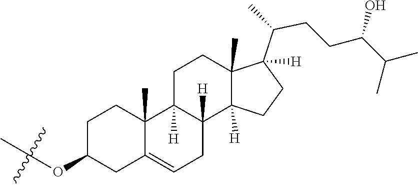 |
| 2068 | 1 | 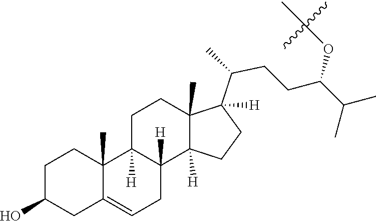 |
| 2069 | 2 | 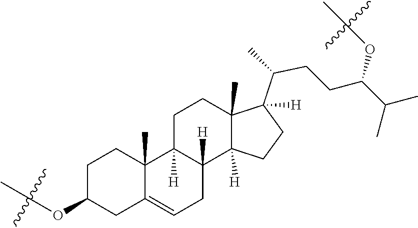 |
| 2070 | 1 | 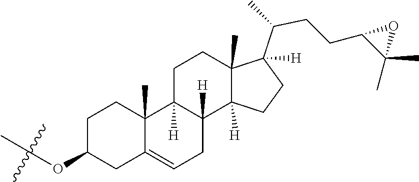 |
| 2071 | 1 | 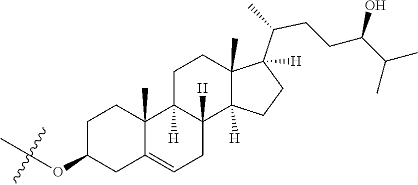 |
| 2072 | 1 | 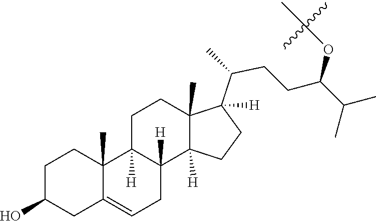 |
| 2073 | 2 | 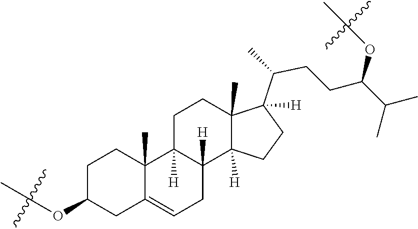 |
| 2074 | 1 | 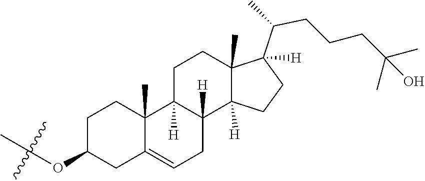 |
| 2075 | 1 | 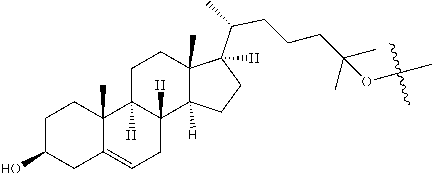 |
| 2076 | 2 | 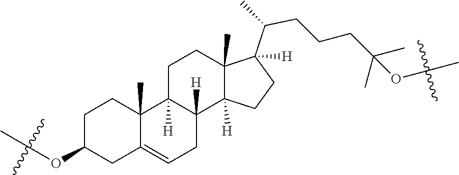 |
| 2077 | 1 | 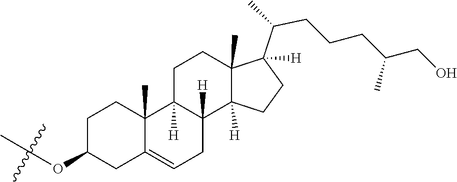 |
| 2078 | 1 | 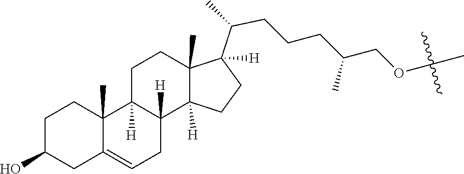 |
| 2079 | 2 | 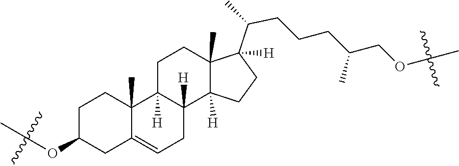 |
| 2080 | 1 | 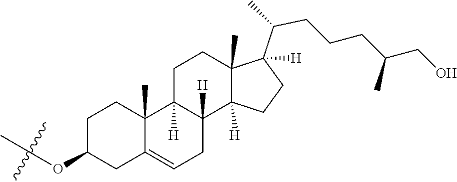 |
| 2081 | 1 | 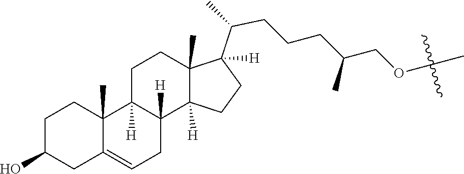 |
| 2082 | 2 | 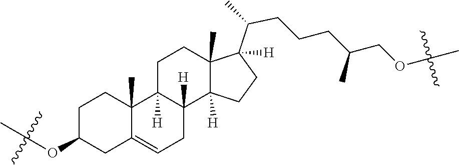 |
| 2083 | 1 | 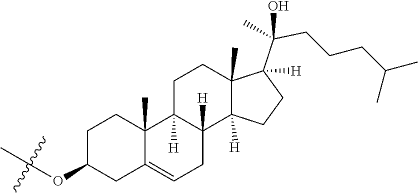 |
| 2084 | 1 | 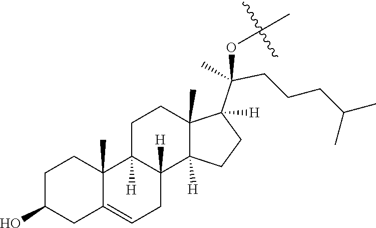 |
| 2085 | 2 | 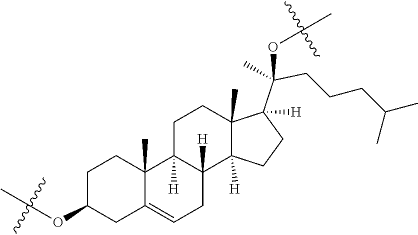 |
| 2086 | 1 | 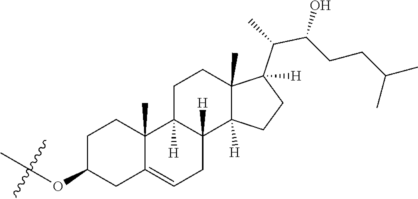 |
| 2087 | 1 | 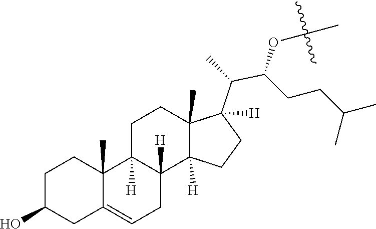 |
| 2088 | 2 | 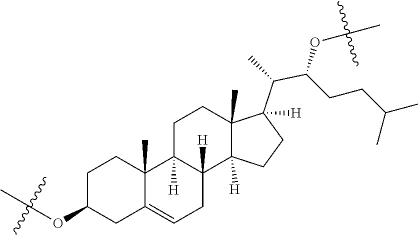 |
| 2089 | 1 | 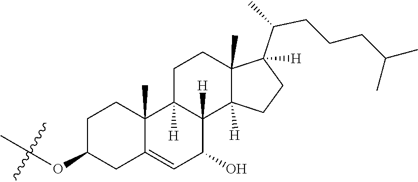 |
| 2090 | 1 | 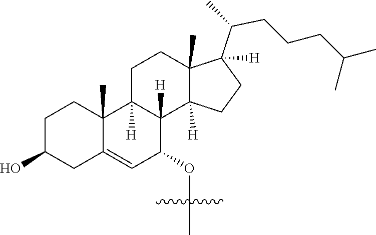 |
| 2091 | 2 | 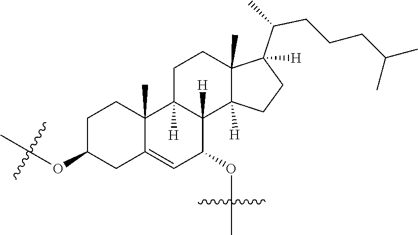 |
| 2092 | 1 | 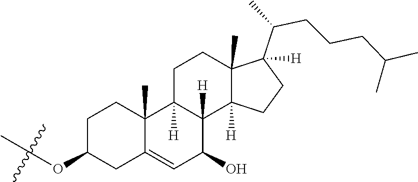 |
| 2093 | 1 | 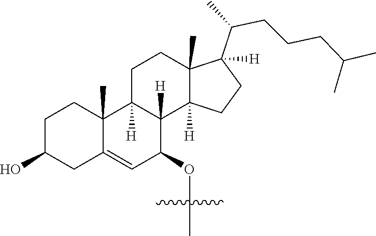 |
| 2094 | 2 | 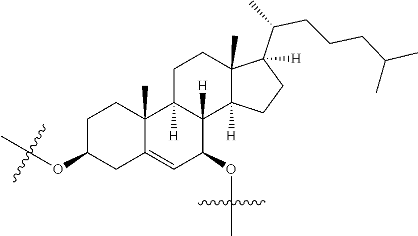 |
| 2095 | 1 | 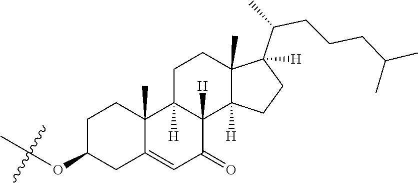 |
| 2096 | 1 | 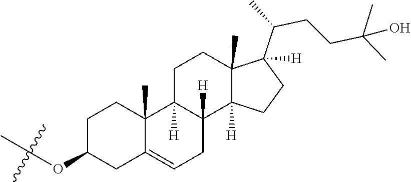 |
| 2097 | 1 | 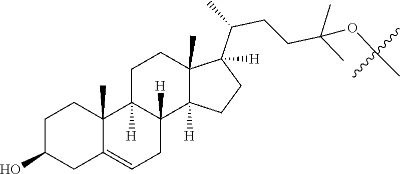 |
| 2098 | 2 | 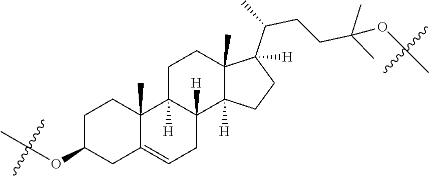 |
| 2099 | 1 | 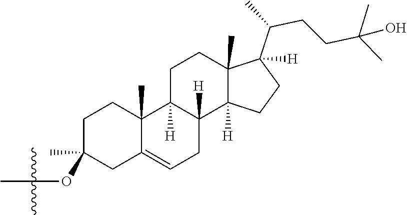 |
| 2100 | 1 | 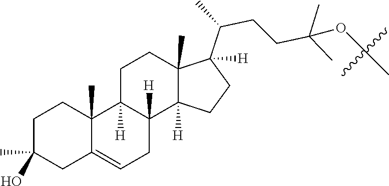 |
| 2101 | 2 | 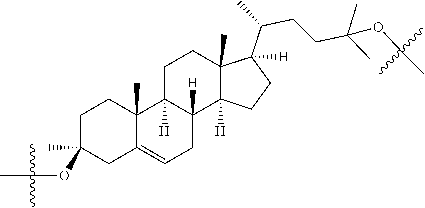 |
| 2102 | 1 | 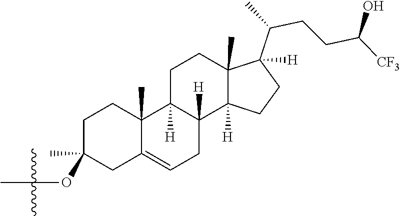 |
| 2103 | 1 | 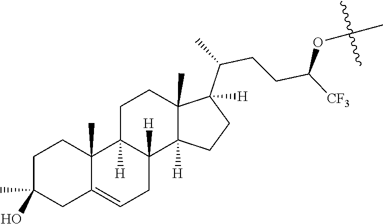 |
| 2104 | 2 | 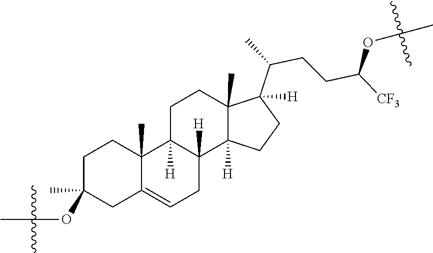 |
| 2105 | 1 | 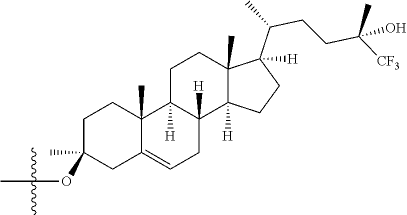 |
| 2106 | 1 | 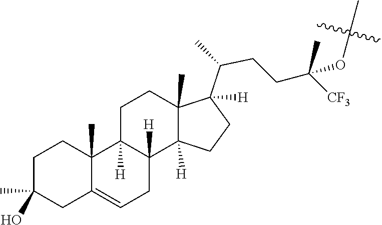 |
| 2107 | 2 | 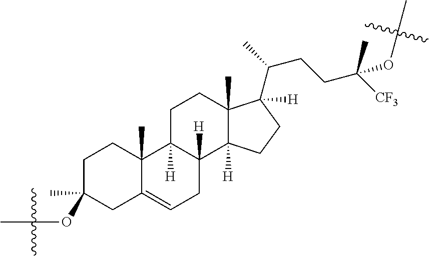 |
| 2108 | 1 | 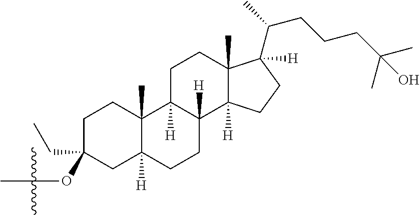 |
| 2109 | 1 | 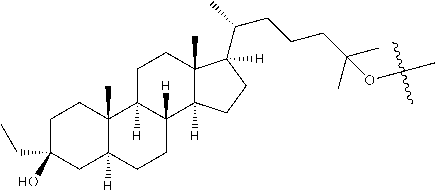 |
| 2110 | 2 | 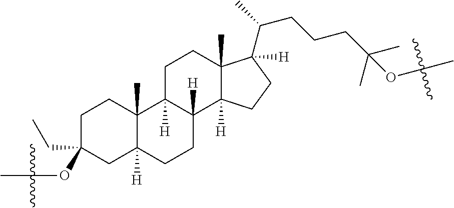 |
| 2111 | 1 | 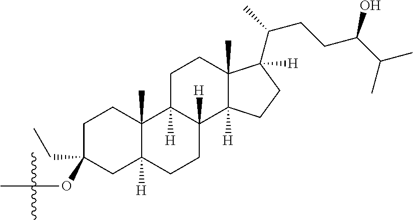 |
| 2112 | 1 | 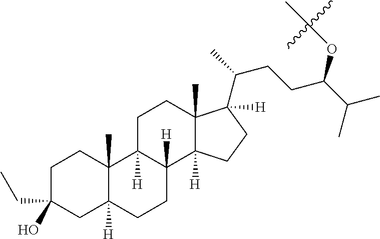 |
| 2113 | 2 | 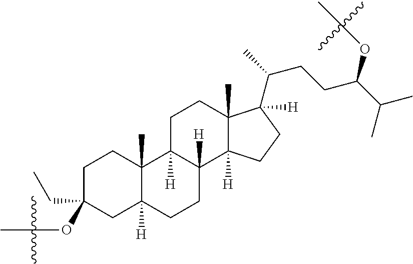 |
| 2114 | 1 | 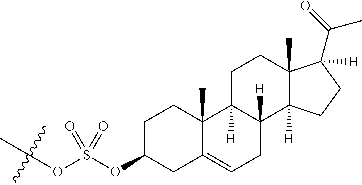 |
| 2115 | 1 | 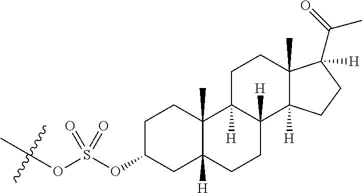 |
| 2116 | 1 | 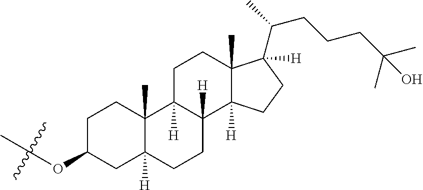 |
| 2117 | 1 | 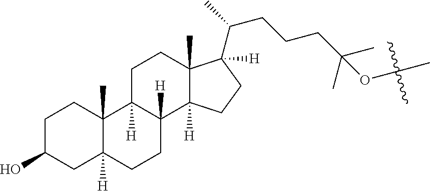 |
| 2118 | 2 | 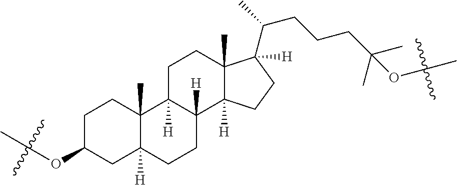 |
| 2119 | 1 | 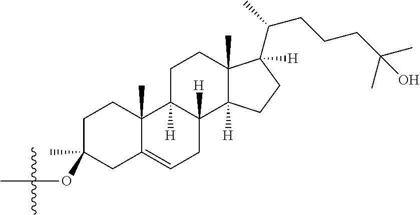 |
| 2120 | 1 | 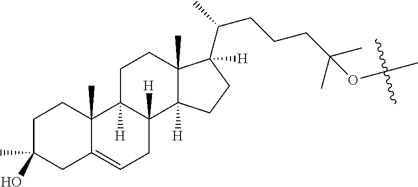 |
| 2121 | 2 | 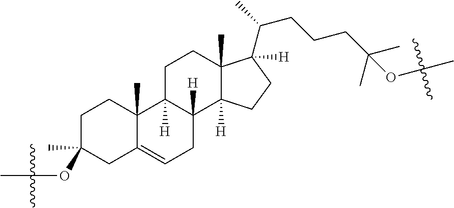 |
| 2122 | 1 | 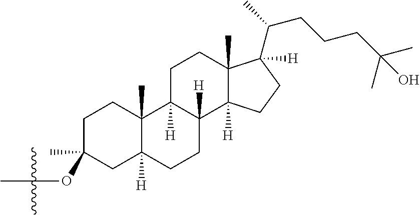 |
| 2123 | 1 | 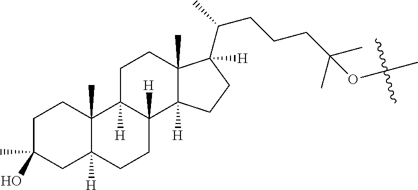 |
| 2124 | 2 | 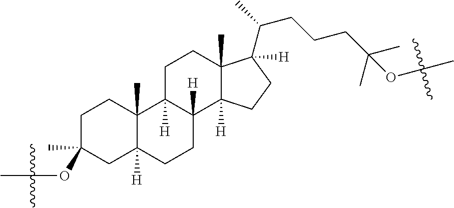 |
| 2125 | 1 | 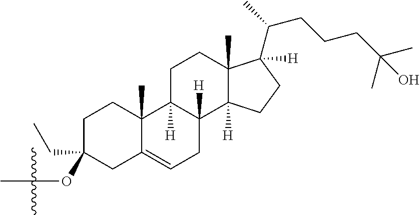 |
| 2126 | 1 | 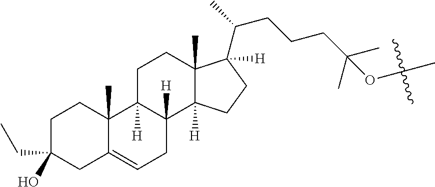 |
| 2127 | 2 | 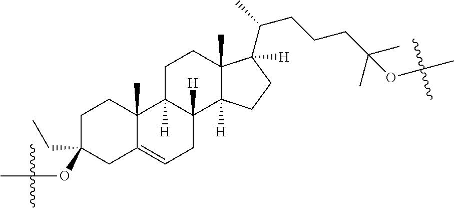 |
| 2128 | 1 | 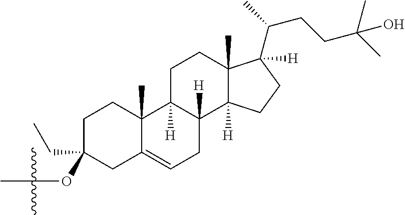 |
| 2129 | 1 | 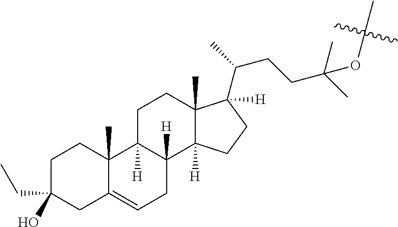 |
| 2130 | 2 | 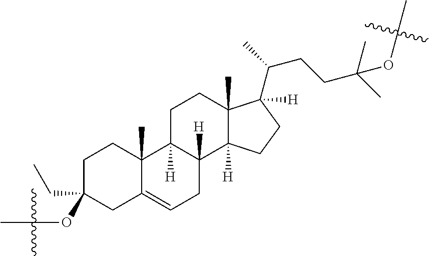 |

In some embodiments, the compound is selected from any of the compounds depicted in in Table 32, pharmaceutically acceptable salts thereof, deuterated variants thereof or combinations thereof.

TABLE 32
Formula (LVIII)

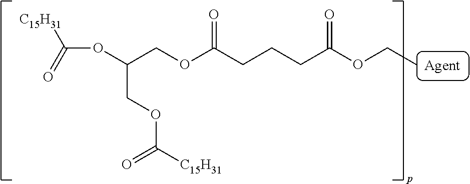

| Cpd. No. | p | 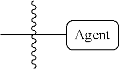 |
|---|---|---|
| 2131 | 1 | 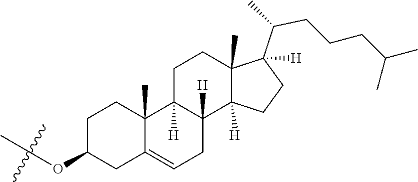 |
| 2132 | 1 | 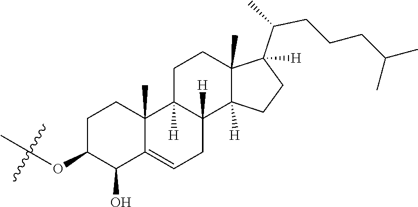 |
| 2133 | 1 | 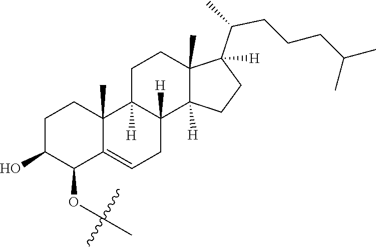 |
| 2134 | 2 | 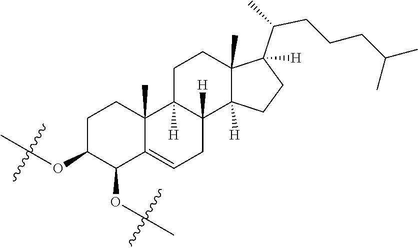 |
| 2135 | 1 | 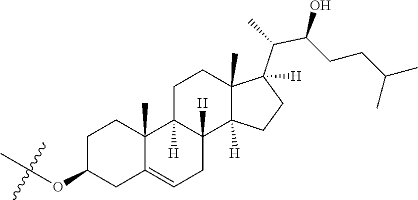 |
| 2136 | 1 | 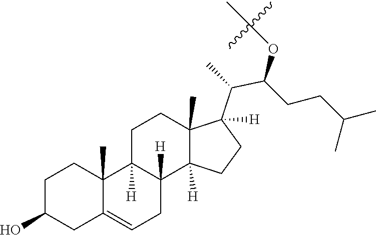 |
| 2137 | 2 | 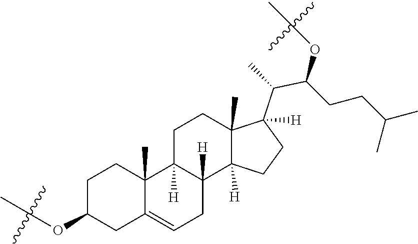 |
| 2138 | 1 | 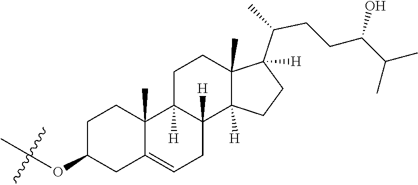 |
| 2139 | 1 | 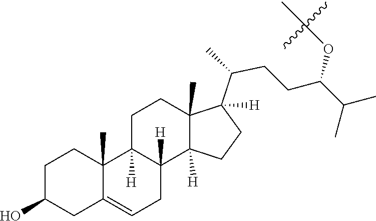 |
| 2140 | 2 | 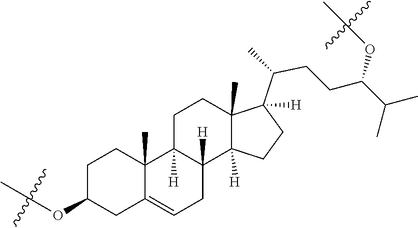 |
| 2141 | 1 | 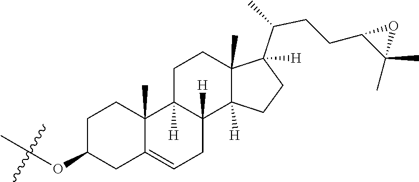 |
| 2142 | 1 | 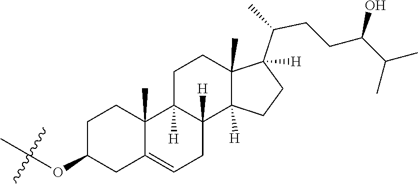 |
| 2143 | 1 | 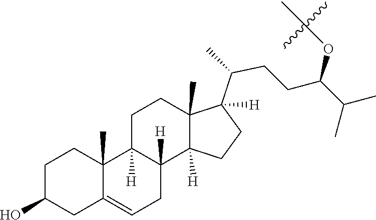 |
| 2144 | 2 | 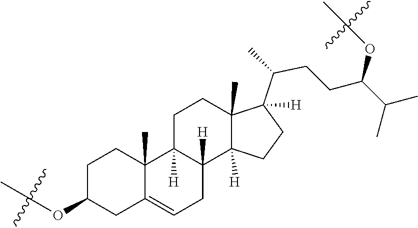 |
| 2145 | 1 | 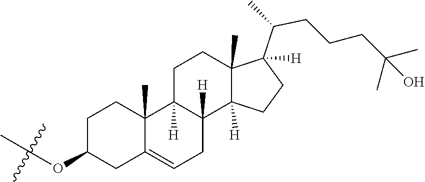 |
| 2146 | 1 | 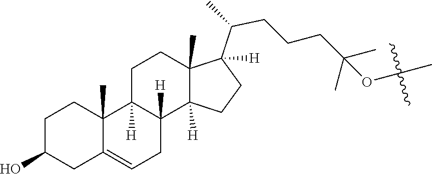 |
| 2147 | 2 | 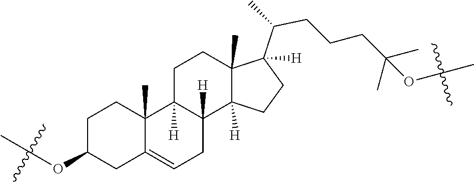 |
| 2148 | 1 | 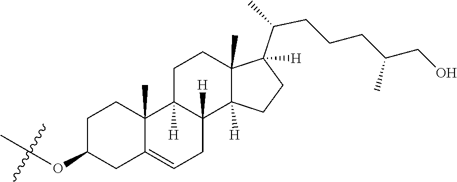 |
| 2149 | 1 | 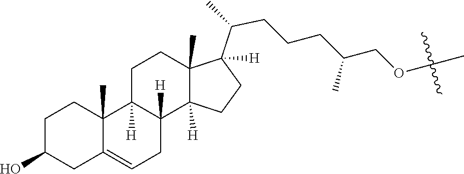 |
| 2150 | 2 | 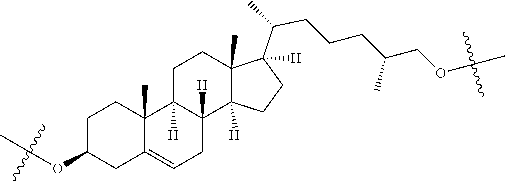 |
| 2151 | 1 | 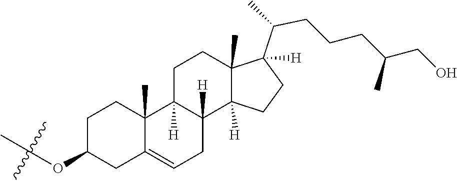 |
| 2152 | 1 | 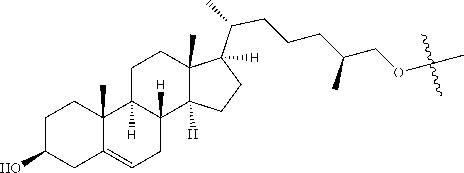 |
| 2153 | 2 | 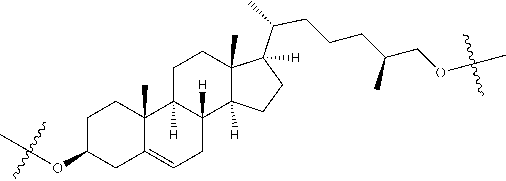 |
| 2154 | 1 | 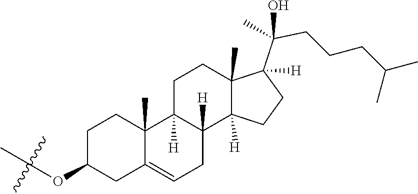 |
| 2155 | 1 | 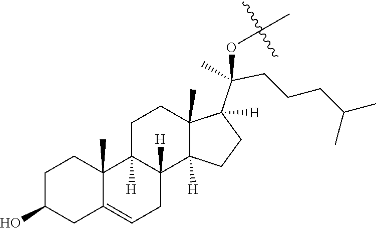 |
| 2156 | 2 | 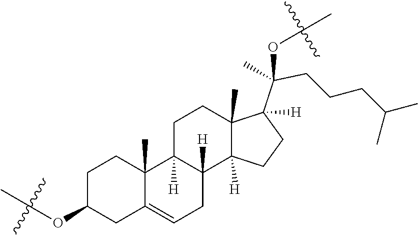 |
| 2157 | 1 | 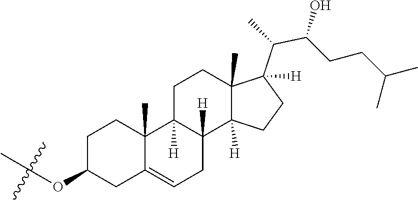 |
| 2158 | 1 | 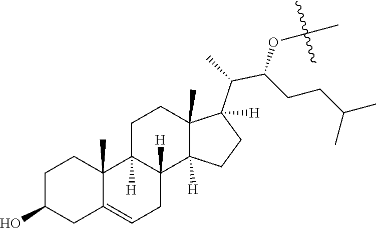 |
| 2159 | 2 | 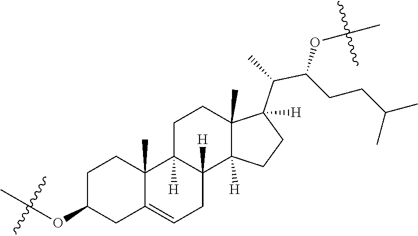 |
| 2160 | 1 | 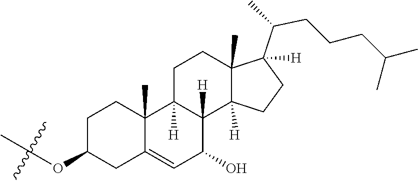 |
| 2161 | 1 | 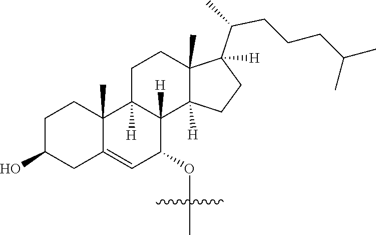 |
| 2162 | 2 | 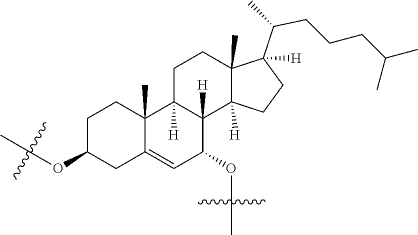 |
| 2163 | 1 | 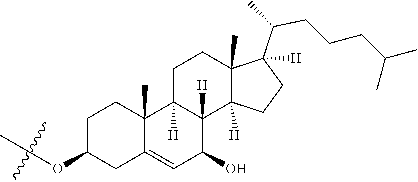 |
| 2164 | 1 | 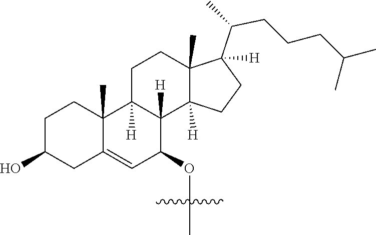 |
| 2165 | 2 | 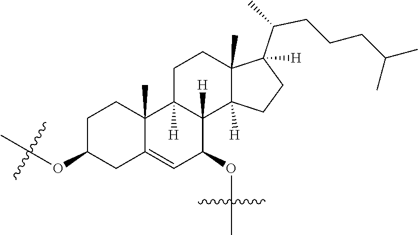 |
| 2166 | 1 | 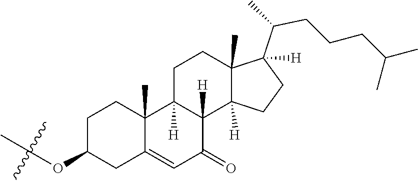 |
| 2167 | 1 | 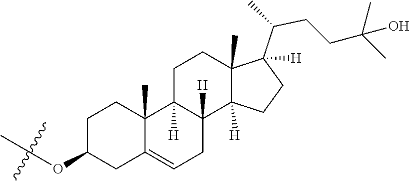 |
| 2168 | 1 | 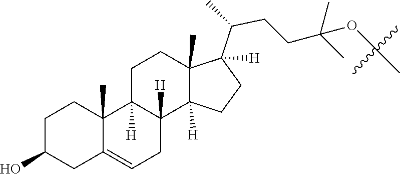 |
| 2169 | 2 | 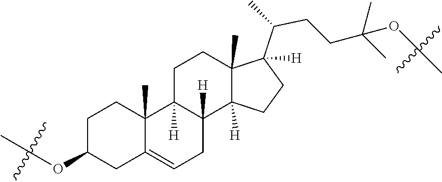 |
| 2170 | 1 | 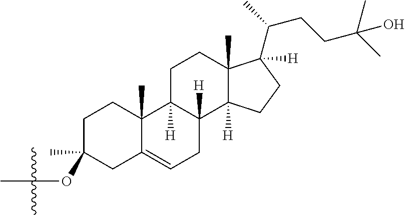 |
| 2171 | 1 | 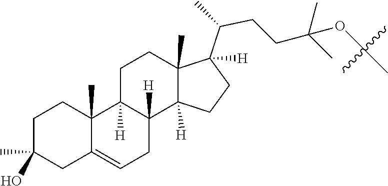 |
| 2172 | 2 | 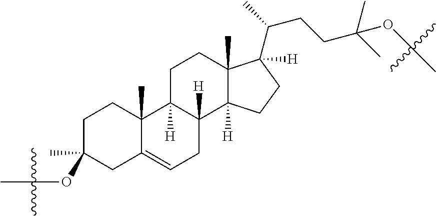 |
| 2173 | 1 | 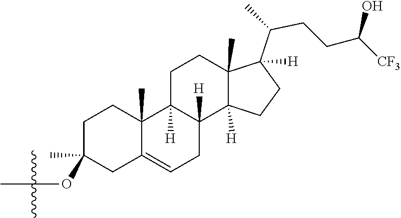 |
| 2174 | 1 | 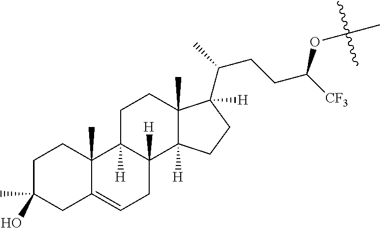 |
| 2175 | 2 | 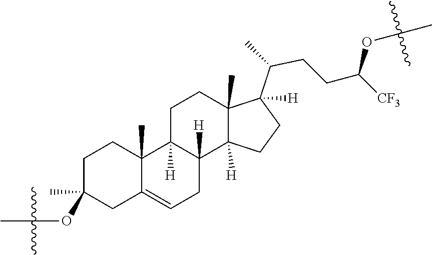 |
| 2176 | 1 | 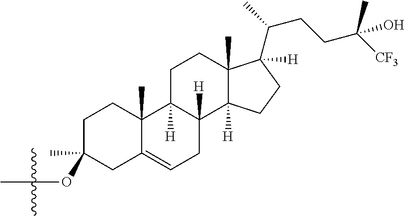 |
| 2177 | 1 | 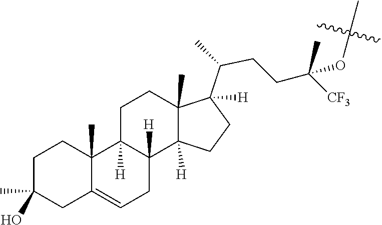 |
| 2178 | 2 | 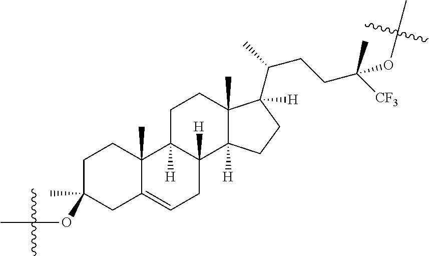 |
| 2179 | 1 | 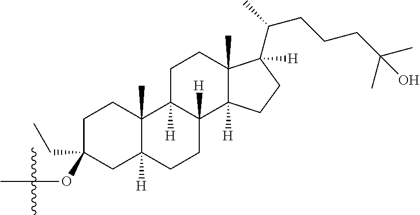 |
| 2180 | 1 | 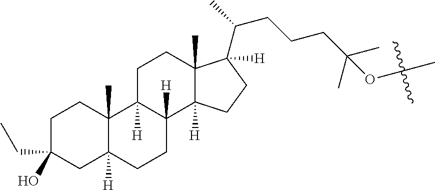 |
| 2181 | 2 | 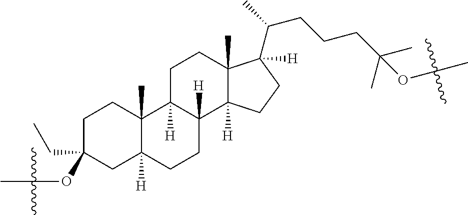 |
| 2182 | 1 | 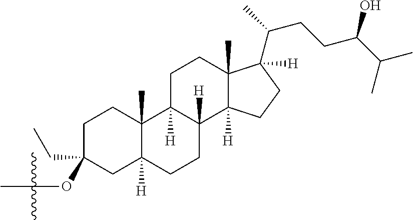 |
| 2183 | 1 | 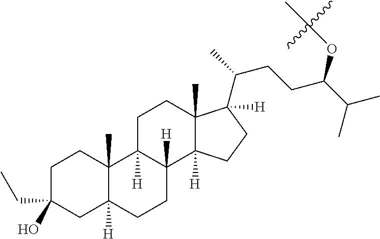 |
| 2184 | 2 | 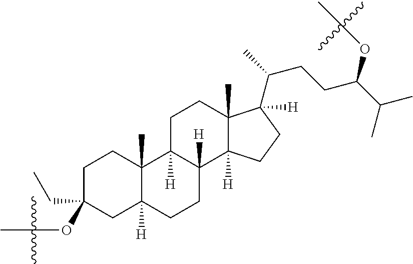 |
| 2185 | 1 | 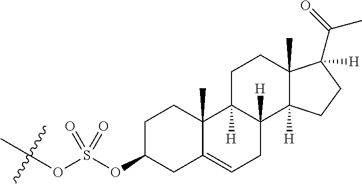 |
| 2186 | 1 | 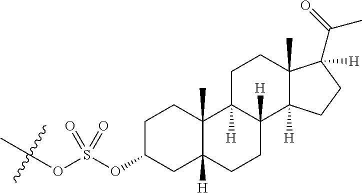 |
| 2187 | 1 | 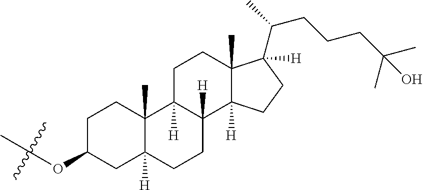 |
| 2188 | 1 | 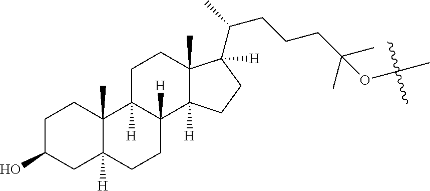 |
| 2189 | 2 | 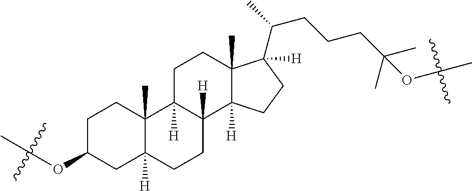 |
| 2190 | 1 | 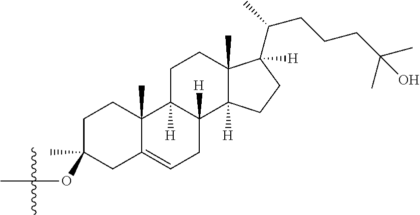 |
| 2191 | 1 | 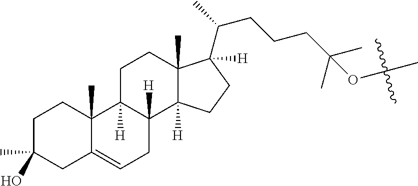 |
| 2192 | 2 | 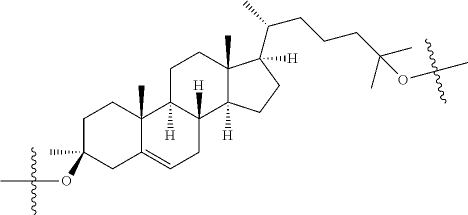 |
| 2193 | 1 | 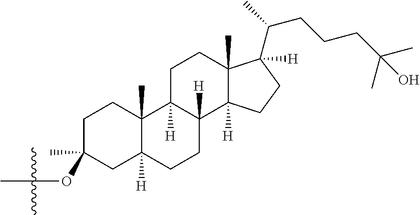 |
| 2194 | 1 | 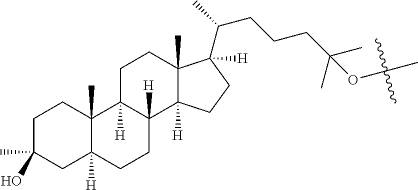 |
| 2195 | 2 | 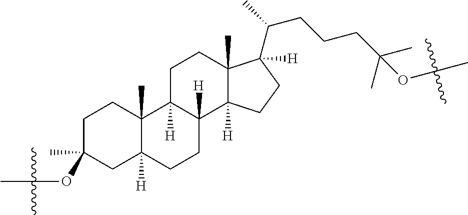 |
| 2196 | 1 | 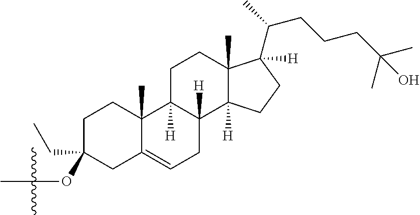 |
| 2197 | 1 | 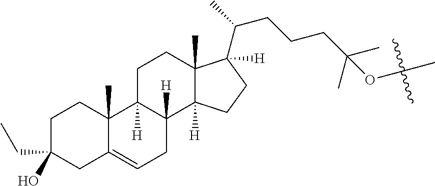 |
| 2198 | 2 | 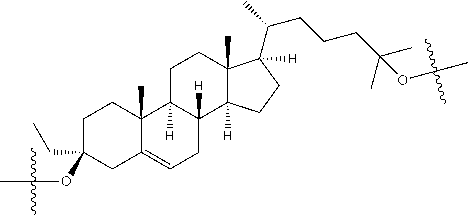 |
| 2199 | 1 | 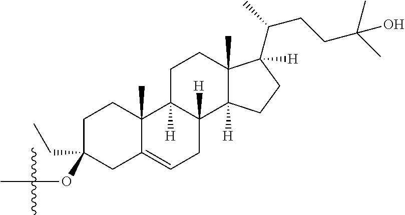 |
| 2200 | 1 | 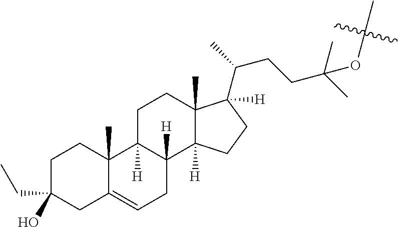 |
| 2201 | 2 | 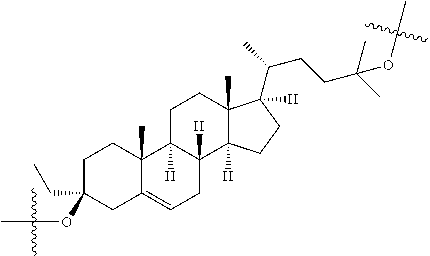 |

### Pharmaceutical Compositions

In one aspect, the disclosure provides a pharmaceutical composition comprising the compounds (e.g., compounds of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XLVIII), (XLIX), (L), (LI), (LII), LIII), (LIV), (LV), (LVI), (LVII), (LVIII), or pharmaceutically acceptable salts thereof disclosed herein and a pharmaceutically acceptable carrier.

In some embodiments, the compound or pharmaceutically acceptable salt thereof of the present disclosure is provided in an effective amount in the pharmaceutical composition. In some embodiments, the compound or pharmaceutically acceptable salt thereof of the present disclosure is provided in a therapeutically effective amount. In some embodiments, the pharmaceutical composition comprises an effective amount of the compound or pharmaceutically acceptable salt thereof. In some embodiments, the pharmaceutical composition comprises a therapeutically effective amount of the compound or pharmaceutically acceptable salt thereof.

The pharmaceutical compositions provided herein can be administered by a variety of routes including oral, parenteral, rectal, transdermal, intradermal, intrathecal, subcutaneous, intravenous, intramuscular, and intranasal.

The amount of the compound administered to the subject is typically determined by a physician, in the light of relevant circumstances, including the condition to be treated, the route of administration, the actual compound to be administered, the age, weight, and response of the individual subject, the severity of the subject's symptoms, and the like.

When used to prevent the onset of a disease, condition or disorder, the compounds provided herein are administered to a subject at risk for developing the disease, condition or disorder, typically on the advice and under the supervision of a physician, and at the dosages described herein. Subjects at risk for developing a particular condition include, but are not limited to, those having a family history of the condition, or those who have been identified by a screening or evaluation method to be particularly susceptible to developing the condition.

The pharmaceutical compositions provided herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or indefinitely (e.g., for the rest of the subject's life).

In some embodiments, the pharmaceutical composition of the present disclosure is formulated for oral administration to a subject. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

With oral dosing, typically, one to five doses (e.g., 1, 2, 3, 4, 5 doses) per day are representative regimens. Using these dosing patterns, each dose provides from about 0.01 to about 20 mg/kg of the compound provided herein.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In some embodiments, the pharmaceutical composition of the present disclosure is formulated for oral administration to a subject as a lipid-based formulation. Lipid-based formulations for oral administration are known in the art and typically include one or more lipid components which may be classified according to the Lipid Formulation Classification System (LFCS). Examples of lipid formulations that may be used in the lipid formulations disclosed herein include the Type I, Type II, Type, III and Type IV formulations per the LFCS classification system. (Pouton, Eur. J. Pharm. Sci. 11 (Supp 2), S93-S98, 2000; Pouton, Eur. J. Pharm. Sci. 29 278-287, 2006). The lipid-based pharmaceutical compositions may contain any one or more of oils or lipids, surfactants, co-surfactants, co-emulsifiers, co-solvents, antioxidants and/or solidifying agents.

The lipid-based pharmaceutical compositions may be chosen formulated to provide for sustained release of the active in the gastrointestinal tract in order to control the rate of absorption. These methodologies and formulations are known in the art. See, e.g., Mishra, Handbook of Encapsulation and Controlled Release, CRC Press, Boca Raton, (2016); Wilson and Crowley Controlled Release in Oral Drug Delivery, Springer, NY (2011); Wise, Handbook of Pharmaceutical Controlled Release Technology, Marcel Dekker, NY, (2000).

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. Injectable compositions can be sterilized, e.g., by filtration, or by incorporating sterilizing agents in the form of sterile solid compositions which can be dissolved or dispersed in sterile water or other sterile injectable medium prior to use.

Transdermal doses are generally selected to provide similar or lower blood levels than are achieved using injection doses.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The present disclosure also relates to the pharmaceutically acceptable acid addition salt of a compound or Agent as disclosed herein. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The present disclosure also provides a pharmaceutical composition comprising a compound of the present invention and a pharmaceutically acceptable carrier, e.g., a composition suitable for injection, such as for intravenous (IV) administration, or for oral administration.

Pharmaceutically acceptable carriers include any and all diluents or other liquid vehicles, dispersion or suspension aids, surface active agents, isotonic agents, preservatives, lubricants and the like, as suited to the particular dosage form desired, e.g., injection, oral, etc. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

The present disclosure also relates to pharmaceutical compositions comprising a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol®. See, e.g., U.S. Pat. No. 5,376,645. In certain embodiments, the formulation comprises hexapropyl-β-cyclodextrin. In a more particular embodiment, the formulation comprises hexapropyl-β-cyclodextrin (10-50% in water).

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

The above-described components for orally administrable, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

The compounds of this disclosure can also be administered in sustained release forms or from sustained release drug delivery systems. A description of representative sustained release materials can be found in Remington's Pharmaceutical Sciences.

### Methods of Treatment and Uses

In one aspect, the disclosure provides compounds (e.g., a compound of Formulae (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XLVIII), (XLIX), (L), (LI), (LII), LIII), (LIV), (LV), (LVI), (LVII), (LVIII), or pharmaceutically acceptable salts thereof) and pharmaceutical compositions thereof that are lymphatic system targeting compounds. In some embodiments, the compounds of Formula (I)-(LVIII), pharmaceutically acceptable salts thereof and pharmaceutical compositions disclosed herein and are useful for delivering an Agent of the disclosure to the lymphatic system and releasing the Agent at a target site (e.g., any target tissue). In some embodiments, the compounds of Formula (I)-(LVIII), pharmaceutically acceptable salts thereof and pharmaceutical compositions disclosed herein and are useful for delivering an Agent of the disclosure to the intestinal lymphatic system and release of the Agent at a target site (e.g., lymph, lymphocytes, lymphoid tissues, tissues with high lipase activity, the liver, the systemic circulation, brain). The compounds of Formula (I)-(LVIII), pharmaceutically acceptable salt thereof, and pharmaceutical compositions thereof disclosed herein are useful for transporting and releasing an Agent as disclosed herein at at a target tissue by avoiding first pass metabolism. The compounds of Formula (I)-(LVIII), pharmaceutically acceptable salt thereof, and pharmaceutical compositions thereof disclosed herein are useful for improving the bioavailability and other pharmacokinetic characteristics of an Agent as disclosed herein.

In some embodiments, the compounds of Formula (I)-(LVIII), or pharmaceutically acceptable salts thereof as disclosed herein is delivered to the central nervous system (CNS). In some embodiments, the compounds of Formula (I)-(LVIII), or pharmaceutically acceptable salts thereof as disclosed herein crosses the blood-brain barrier (BBB) via the lymphatic system.

In some embodiments, the compounds of Formula (I)-(LVIII), or pharmaceutically acceptable salts thereof as disclosed herein are cleaved releasing an Agent as disclosed herein, after the compounds of Formula (I)-(LVIII), or pharmaceutically acceptable salts thereof as disclosed herein reaches the target tissue. The Agent is released when the cleavable linker is cleaved.

The compounds (e.g., a compound of Formulae (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XLVIII), (XLIX), (L), (LI), (LII), LIII), (LIV), (LV), (LVI), (LVII), (LVIII), or a pharmaceutically acceptable salt thereof) and Agents of the present disclosure (e.g., Agents 1-27, deuterated variants thereof, pharmaceutically acceptable salts of the Agents or pharmaceutically acceptable salts of the deuterated variants of Agents), as described herein, are generally designed to be positive allosteric modulators of NMDA receptor function, and therefore are useful for the treatment and prevention of, e.g., CNS-related conditions in a subject.

In some embodiments, the compounds, Agents, pharmaceutically acceptable salts, deuterated variants, or combinations thereof described herein, are generally designed to penetrate the blood brain barrier (e.g., designed to be transported across the blood brain barrier).

In certain embodiments, the compound, Agent, pharmaceutically acceptable salt, deuterated variant, or combination thereof of the present disclosure is a positive allosteric modulator (PAM) of NMDA receptors and activates NMDA receptor function. In certain embodiments, the compound, Agent, pharmaceutically acceptable salt, deuterated variant, or combination thereof of the present disclosure is a negative allosteric modulator (NAM) of an NMDA receptor and inhibits NMDA receptor function. In certain embodiments, the compound, Agent, pharmaceutically acceptable salt, deuterated variant, or combination thereof, is a neutral allosteric ligands (NALs) of NMDA receptors, which binds at allosteric sites and block the effects of a PAM and/or NAM of an NMDA receptor.

In one aspect, the disclosure provides a method for effecting allosteric modulation of an NMDA receptor in a subject, comprising administering to the subject an effective amount of a compound, Agent, pharmaceutically acceptable salt, deuterated variant, or combination thereof or pharmaceutical composition as disclosed herein. In some embodiments, the method is for effecting positive allosteric modulation of an NMDA receptor in a subject. In some embodiments, the method is for effecting negative allosteric modulation of an NMDA receptor in a subject. In some embodiments, the method comprises binding an NMDA receptor at an allosteric site with a compound disclosed herein and blocking the effects of an allosteric NMDA modulator (i.e., PAM and/or NAM) in a subject.

In one aspect, the disclosure provides a method for treating a disease, disorder, or condition requiring allosteric NMDA receptor modulation in a subject, comprising administering to the subject an effective amount of a compound, Agent, pharmaceutically acceptable salt, deuterated variant, or combination thereof or pharmaceutical composition thereof as disclosed herein. In some embodiments, treating the disease, disorder, or condition requires positive allosteric NMDA receptor modulation in a subject. In some embodiments, treating the disease, disorder, or condition requires negative allosteric NMDA receptor modulation in a subject.

In one aspect, the disclosure provides a method for treating a CNS-related condition in a subject, comprising administering to the subject an effective amount of a compound, Agent, pharmaceutically acceptable salt, deuterated variant, or combination thereof or a pharmaceutical composition thereof as disclosed herein.

In one aspect, the disclosure provides a method for preventing a disease, disorder or condition requiring allosteric NMDA receptor modulation in a subject, comprising administering to the subject an effective amount of a compound, Agent, pharmaceutically acceptable salt, deuterated variant, or combination thereof or a pharmaceutical composition thereof as disclosed herein. In some embodiments, preventing the disease, disorder, or condition requires positive allosteric NMDA receptor modulation in a subject. In some embodiments, preventing the disease, disorder, or condition requires negative allosteric NMDA receptor modulation in a subject.

In one aspect, the disclosure provides a method for preventing a CNS-related condition in a subject, comprising administering to the subject an effective amount of a compound, Agent, pharmaceutically acceptable salt, deuterated variant, or combination thereof or a pharmaceutical composition thereof as disclosed herein.

In one aspect, the disclosure provides a method for inducing sedation or anesthesia in a subject, comprising administering to the subject an effective amount of a compound, Agent a pharmaceutically acceptable salt, deuterated variant, or combination thereof or a pharmaceutical composition thereof as disclosed herein.

In one aspect, the disclosure provides a compound, Agent, pharmaceutically acceptable salt, deuterated variant, or combination thereof or pharmaceutical composition thereof as disclosed herein for use in effecting allosteric modulation of an NMDA receptor in a subject. In some embodiments, the compound, Agent, pharmaceutically acceptable salt, deuterated variant, or combination thereof or pharmaceutical composition is for use in effecting positive allosteric modulation of an NMDA receptor in a subject. In some embodiments, the compound, Agent, pharmaceutically acceptable salt, deuterated variant, or combination thereof or pharmaceutical composition is for use in effecting negative allosteric modulation of an NMDA receptor in a subject.

In one aspect, the disclosure provides a compound, Agent, pharmaceutically acceptable salt, deuterated variant, or combination thereof or pharmaceutical composition as disclosed herein for use in treating a disease, disorder or condition requiring allosteric NMDA receptor modulation in a subject. In some embodiments, treating the disease, disorder, or condition requires positive allosteric NMDA receptor modulation in a subject. In some embodiments, treating the disease, disorder, or condition requires negative allosteric NMDA receptor modulation in a subject.

In one aspect, the disclosure provides a compound, Agent, pharmaceutically acceptable salt, deuterated variant, or combination thereof or pharmaceutical composition as disclosed herein for use in treating a CNS-related condition in a subject.

In one aspect, the disclosure provides a compound, Agent, pharmaceutically acceptable salt, deuterated variant, or combination thereof or pharmaceutical composition as disclosed herein for use in preventing a disease, disorder or condition requiring allosteric NMDA receptor modulation in a subject. In some embodiments, preventing the disease, disorder, or condition requires positive allosteric NMDA receptor modulation in a subject. In some embodiments, preventing the disease, disorder, or condition requires negative allosteric NMDA receptor modulation in a subject.

In one aspect, the disclosure provides a compound, Agent, pharmaceutically acceptable salt, deuterated variant, or combination thereof or pharmaceutical composition as disclosed herein for use in preventing a CNS-related condition in a subject.

In one aspect, the disclosure provides a compound, Agent, pharmaceutically acceptable salt, deuterated variant, or combination thereof or pharmaceutical composition as disclosed herein for use in inducing sedation or anesthesia in a subject.

In one aspect, the disclosure provides the use of a compound, Agent, pharmaceutically acceptable salt, deuterated variant, or combination thereof or pharmaceutical composition as disclosed herein for the manufacture of a medicament for effecting allosteric modulation of an NMDA receptor in a subject. In some embodiments, the medicament is for effecting positive allosteric modulation of an NMDA receptor in a subject. In some embodiments, the medicament is for effecting negative allosteric modulation of an NMDA receptor in a subject.

In one aspect, the disclosure provides the use of a compound, Agent, pharmaceutically acceptable salt, deuterated variant, or combination thereof or pharmaceutical composition as disclosed herein for the manufacture of a medicament for treating a disease, disorder or condition requiring allosteric NMDA receptor modulation in a subject. In some embodiments, treating the disease, disorder, or condition requires positive allosteric NMDA receptor modulation in a subject. In some embodiments, treating the disease, disorder, or condition requires negative allosteric NMDA receptor modulation in a subject.

In one aspect, the disclosure provides the use of a compound, Agent, pharmaceutically acceptable salt, deuterated variant, or combination thereof or pharmaceutical composition as disclosed herein for the manufacture of a medicament for treating a CNS-related condition in a subject.

In one aspect, the disclosure provides the use of a compound, Agent, pharmaceutically acceptable salt, deuterated variant, or combination thereof or pharmaceutical composition as disclosed herein for the manufacture of a medicament for preventing a disease, disorder or condition requiring allosteric NMDA receptor modulation in a subject. In some embodiments, the medicament is for preventing a disease, disorder, or condition requiring positive allosteric NMDA receptor modulation in a subject. In some embodiments, the medicament is for preventing a disease, disorder, or condition requiring negative allosteric NMDA receptor modulation in a subject.

In one aspect, the disclosure provides the use compound, Agent, pharmaceutically acceptable salt, deuterated variant, or combination thereof or pharmaceutical composition as disclosed herein for the manufacture of a medicament for preventing a CNS-related condition in a subject.

In one aspect, the disclosure provides the use of compound, Agent, pharmaceutically acceptable salt, deuterated variant, or combination thereof or pharmaceutical composition as disclosed herein for the manufacture of a medicament for inducing sedation or anesthesia in a subject.

In some embodiments, the method of treating a disease, disorder or condition comprises administering a compound, pharmaceutically acceptable salt or pharmaceutical composition of the disclosure in combination with one or more additional therapeutic agents. In some embodiments, the one or more additional therapeutic agent is administered simultaneously with the compound, pharmaceutically acceptable salt or pharmaceutical composition of the disclosure. In some embodiments, the one or more additional therapeutic agent and the compound, pharmaceutically acceptable salt or pharmaceutical composition of the disclosure are administered sequentially. In some embodiments, the one or more additional therapeutic agent is administered prior to the compound, pharmaceutically acceptable salt or pharmaceutical composition of the disclosure. In some embodiments, the one or more additional therapeutic agent is administered after the compound, pharmaceutically acceptable salt or pharmaceutical composition of the disclosure.

Exemplary CNS conditions related to positive allosteric modulation of NMDA receptors include, but are not limited to, an adjustment disorder, an anxiety disorder (including obsessive-compulsive disorder, posttraumatic stress disorder, and social phobia), a cognitive disorder (including Alzheimer's disease and other forms of dementia), a dissociative disorder, an eating disorder, a mood disorder (including depression, bipolar disorder, and dysthymic disorder), schizophrenia or another psychotic disorder (including schizoaffective disorder and post-partum psychosis), a sleep disorder (including insomnia), a substance-related disorder, a personality disorder (including obsessive-compulsive personality disorder), an autism spectrum disorder (including those involving mutations to the Shank group of proteins), multiple sclerosis, a neurodevelopmental disorder (including Rett syndrome, Tuberous Sclerosis complex), attention deficit disorder, attention deficit hyperactivity disorder, pain (including acute and chronic pain), a metabolic encephalopathies (including phenylketonuria), an encephalopathy secondary to a medical condition (including hepatic encephalopathy and anti-NMDA receptor encephalitis), a seizure disorder (including status epilepticus and monogenic forms of epilepsy such as Dravet's disease), stroke, traumatic brain injury, a movement disorder (including Huntington's disease and Parkinson's disease), and tinnitus.

Exemplary CNS conditions related to negative allosteric modulation of NMDA receptors include, but are not limited to, adjustment disorders, stress or stress disorders (including post-traumatic stress disorder (PTSD)), anxiety disorders (including obsessive-compulsive disorder, posttraumatic stress disorder, social phobia, social anxiety disorder, and generalized anxiety disorder), cognitive disorders (including Alzheimer's disease and other forms of dementia (e.g., frontotemporal dementia), as well as attention disorders such as attention deficit hyperactive disorder (ADHD)), eating disorders, mood disorders (including depression (e.g., postpartum depression), bipolar disorder, dysthymic disorder, suicidality), schizophrenia spectrum disorders (e.g., schizophrenia, schizoaffective disorder), psychotic disorders, sleep disorders (including insomnia), substance abuse-related disorders and/or withdrawal syndromes (e.g., addiction to opiates, cocaine, and/or alcohol), personality disorders (including obsessive-compulsive personality disorder (OCD)), autism spectrum disorders (including those involving mutations to the Shank group of proteins (e.g., Shank3), Rett syndrome, Fragile X syndrome, and Angelman syndrome), addictive disorders, neurodevelopmental disorders (including Rett syndrome), pain (including neuropathic pain, injury-related pain syndromes, acute pain, and chronic pain; headaches, e.g., migraine headaches), seizures (including grand-mal seizures, absence seizures, myoclonic seizures, clonic seizures, tonic seizures, and atonic seizures) and seizure disorders (including status epilepticus and monogenic forms of epilepsy such as Dravet's disease, and Tuberous Sclerosis Complex (TSC)), vascular diseases (e.g., stroke, ischemia, vascular malformations), traumatic brain injury, movement disorders (including Huntington's disease, Parkinson's disease, and tremors), neuropsychiatric lupus, and tinnitus.

Exemplary CNS conditions related to positive allosteric modulation of NMDA receptors include, but are not limited to, an adjustment disorder, an anxiety disorder (including obsessive-compulsive disorder, posttraumatic stress disorder, and social phobia), a cognitive disorder (including Alzheimer's disease and other forms of dementia), a dissociative disorder, an eating disorder, a mood disorder (including depression, bipolar disorder, and dysthymic disorder), schizophrenia or another psychotic disorder (including schizoaffective disorder and post-partum psychosis), a sleep disorder (including insomnia), a substance-related disorder, a personality disorder (including obsessive-compulsive personality disorder), an autism spectrum disorder (including those involving mutations to the Shank group of proteins), multiple sclerosis, a neurodevelopmental disorder (including Rett syndrome, Tuberous Sclerosis complex), attention deficit disorder, attention deficit hyperactivity disorder, pain (including acute and chronic pain), a metabolic encephalopathies (including phenylketonuria), an encephalopathy secondary to a medical condition (including hepatic encephalopathy and anti-NMDA receptor encephalitis), a seizure disorder (including status epilepticus and monogenic forms of epilepsy such as Dravet's disease), stroke, traumatic brain injury, a movement disorder (including Huntington's disease and Parkinson's disease), and tinnitus.

Exemplary CNS conditions related to negative allosteric modulation of NMDA receptors include, but are not limited to, adjustment disorders, stress or stress disorders (including post-traumatic stress disorder (PTSD)), anxiety disorders (including obsessive-compulsive disorder, posttraumatic stress disorder, social phobia, social anxiety disorder, and generalized anxiety disorder), cognitive disorders (including Alzheimer's disease and other forms of dementia (e.g., frontotemporal dementia), as well as attention disorders such as attention deficit hyperactive disorder (ADHD)), eating disorders, mood disorders (including depression (e.g., postpartum depression), bipolar disorder, dysthymic disorder, suicidality), schizophrenia spectrum disorders (e.g., schizophrenia, schizoaffective disorder), psychotic disorders, sleep disorders (including insomnia), substance abuse-related disorders and/or withdrawal syndromes (e.g., addiction to opiates, cocaine, and/or alcohol), personality disorders (including obsessive-compulsive personality disorder (OCD)), autism spectrum disorders (including those involving mutations to the Shank group of proteins (e.g., Shank3), Rett syndrome, Fragile X syndrome, and Angelman syndrome), addictive disorders, neurodevelopmental disorders (including Rett syndrome), pain (including neuropathic pain, injury-related pain syndromes, acute pain, and chronic pain; headaches, e.g., migraine headaches), seizures (including grand-mal seizures, absence seizures, myoclonic seizures, clonic seizures, tonic seizures, and atonic seizures) and seizure disorders (including status epilepticus and monogenic forms of epilepsy such as Dravet's disease, and Tuberous Sclerosis Complex (TSC)), vascular diseases (e.g., stroke, ischemia, vascular malformations), traumatic brain injury, movement disorders (including Huntington's disease, Parkinson's disease, and tremors), neuropsychiatric lupus, and tinnitus.

### Dosing Regimens

In one aspect, the disclosure provides a dosing regimen lymphatic uptake, metabolism, and release of the Agent. For example, if a given dose of a compound of the disclosure is absorbed more efficiently than an equivalent dose of the Agent alone, the dose of the compound may be adjusted so as to provide the desired plasma or lymphatic system concentration of the Agent. In some embodiments, the dose of the compound, or pharmaceutically acceptable salt thereof of the disclosure is selected such that when said compound, or pharmaceutically acceptable salt thereof is administered to the subject, it provides, upon lymphatic uptake, metabolism, and release of the Agent, a desired, effective concentration or dose of Agent to treat a disease, disorder, or condition disclosed herein.

In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of Agent No. 18 of about 0.3 mg to about 2.1 mg; about 0.3 mg to about 1.8 mg, about 0.3 mg to about 1.5 mg, about 0.3 mg to about 1.2 mg, about 0.6 mg to about 1.2 mg, about 0.6 mg to about 1.8 mg about 0.9 mg to about 1.2 mg, or about 0.9 mg to about 1.5 mg. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of about 0.3 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of about 0.6 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of about 0.9 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of about 1.2 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of about 1.5 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of about 1.8 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a dose of about 2.1 mg of Agent No. 18.

In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965,2034-2036, 2105-2107 or 2176-2178) of the disclosure is administered to the subject in an amount sufficient to provide a total daily dose of Agent No. 18 of about 0.3 mg to about 2.1 mg; about 0.3 mg to about 1.8 mg, about 0.3 mg to about 1.5 mg, about 0.3 mg to about 1.2 mg, about 0.6 mg to about 1.2 mg, about 0.6 mg to about 1.8 mg about 0.9 mg to about 1.2 mg, or about 0.9 mg to about 1.5 mg. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a total daily dose of about 0.3 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a total daily dose of about 0.6 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a total daily dose of about 0.9 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a total daily dose of about 1.2 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a total daily dose of about 1.5 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a total daily dose of about 1.8 mg of Agent No. 18. In some embodiments, a compound or pharmaceutically acceptable salt thereof (e.g., compounds 46-48, 117-119, 188-190, 259-261, 330-332, 401-403, 472-474, 543-545, 614-616, 685-687, 756-768, 827-829, 898-900, 969-971, 1040-1042, 1111-1113, 1182-1184, 1253-1255, 1324-1326, 1395-1397, 1466-1468, 1537-1539, 1608-1610, 1679-1681, 1750-1752, 1821-1823, 1892-1894, 1963-1965, 2034-2036, 2105-2107 or 2176-2178) of the disclosure is administered to the subject in an amount sufficient to provide a total daily dose of about 2.1 mg of Agent No. 18.

### EXAMPLES

In order that the disclosure described herein may be more fully understood, the following examples are set forth. The synthetic and biological examples described in this application are offered to illustrate the compounds, pharmaceutical compositions and methods provided herein and are not to be construed in any way as limiting their scope.

The absolute configuration of an asymmetric center can be determined using methods known to one skilled in the art. In some embodiments, the absolute configuration of an asymmetric center in a compound can be elucidated from the X-ray single-crystal structure of the compound. In some embodiments, the absolute configuration of an asymmetric center elucidated by the X-ray crystal structure of a compound can be used to infer the absolute configuration of a corresponding asymmetric center in another compound obtained from the same or similar synthetic methodologies. In some embodiments, the absolute configuration of an asymmetric center elucidated by the X-ray crystal structure of a compound can be used to infer the absolute configuration of a corresponding asymmetric center in another compound coupled with a spectroscopic technique, e.g., NMR spectroscopy, e.g., ¹H NMR spectroscopy or ¹⁹F NMR spectroscopy.

### Abbreviations

CSA: Camphorsulphonic acid; DMAP: 4-dimethylaminopyridine; DCM: dichloromethane; DCC: Dicyclohexylcarbodiimide; TBS: t-Butyldimethylsilyl; MeOH: Methanol; EtOAc: Ethyl Acetate; ACN: Acetonitrile; AgOTf: Silver trifluoromethanesulfonate; DCE: 1,2-Dichloroethane; THIF: Tetrahydrofuran; TBAF: Tetra-n-butylammonium fluoride; DMP: Dess-Martin Periodinane.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative compounds that have been disclosed herein. The compounds provided herein may be prepared from known or commercially available starting materials and reagents by one skilled in the art of organic synthesis. Exemplary chiral columns available for use in the separation/purification of the enantiomers/diastereomers provided herein include, but are not limited to, CHIRALPAK® AD-10, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

1H-NMR reported herein (e.g., for the region between δ (ppm) of about 0.5 to about 4 ppm) will be understood to be an exemplary interpretation of the NMR spectrum (e.g., exemplary peak integrations) of a compound. Exemplary general method for preparative HPLC: Column: Waters RBridge prep 10 m C18, 19*250 mm. Mobile phase: acetonitrile, water (NH₄HCO₃) (30 L water, 24 g NH₄HCO₃, 30 mL NH₃·H₂O). Flow rate: 25 mL/min.

Exemplary general method for analytical HPLC: Mobile phase: A: water (10 mM NH₄HCO₃, B: acetonitrile Gradient: 5%-95% B in 1.6 or 2 min Flow rate: 1.8 or 2 mL/min; Column: XBridge C18, 4.6*50 mm, 3.5 μm at 45 C.

Exemplary general method for SFC: Column: CHIRALPAK® AD CSP (250 mm*30 mm, 10 m), Gradient: 45% B, A=NH₃H₂O, B=MeOH, flow rate: 60 mL/min. For example, AD_3_EtOH_DEA_5_40_25ML would indicate: “Column: Chiralpak AD-3 150×4.6 mm I.D., 3 um Mobile phase: A: CO2 B:ethanol (0.05% DEA) Gradient: from 5% to 40% of B in 5 min and hold 40% for 2.5 min, then 5% of B for 2.5 min Flow rate: 2.5 mL/min Column temp: 35° C.

### Example 1: Exemplary General Method for the Synthesis of a Compound of the Disclosure

PG=protecting group.

Carboxylic acids of formula (i), which can be prepared according to procedures know in the art, are dissolved in dichloromethane, treated with di-methylaminopyridine followed by addition of the Agent. If the Agent comprises two hydroxyl groups, one of the hydroxyl groups is protected and only the free hydroxyl group will react with the carboxylic acid of formula (ii) to form a compound of formula (iii). See Panel A. If neither of the hydroxyl groups of the Agent are protected, both can react with two equivalents of the carboxylic acid of formula (i), to form a compound of formula (iv). See Panel B.

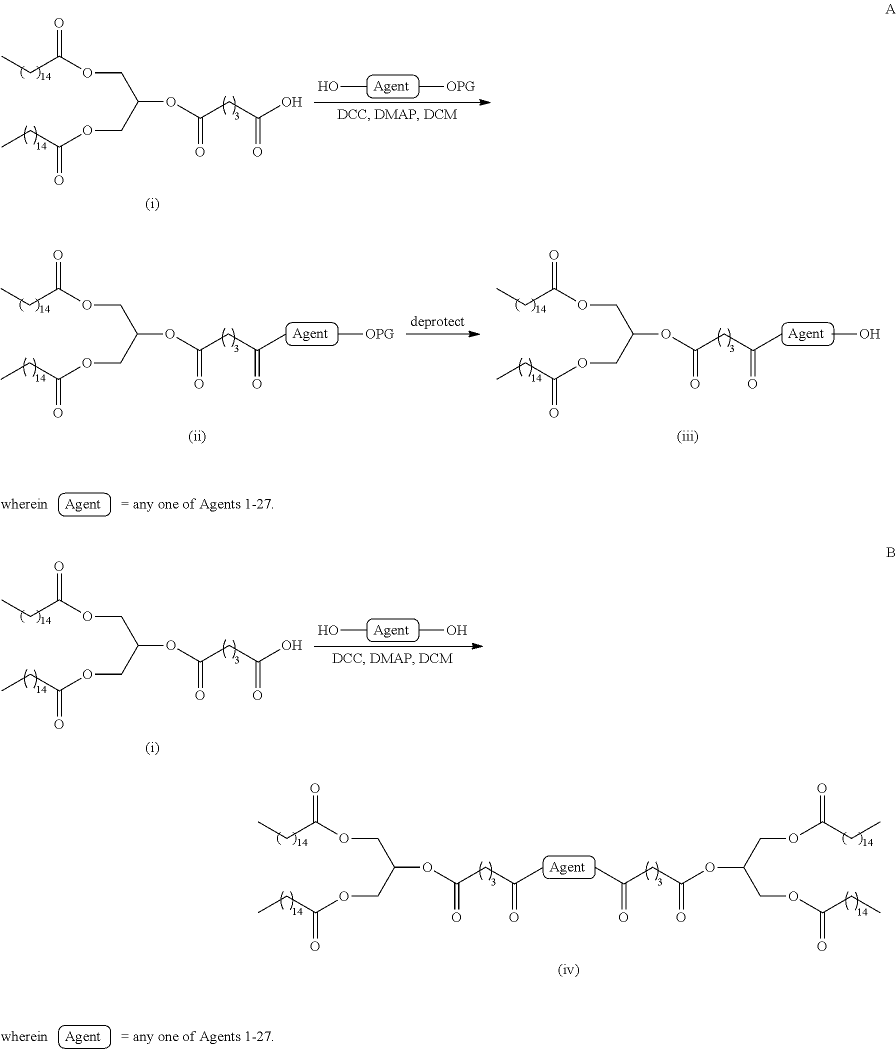

### Example 2: Synthesis of Compound 9

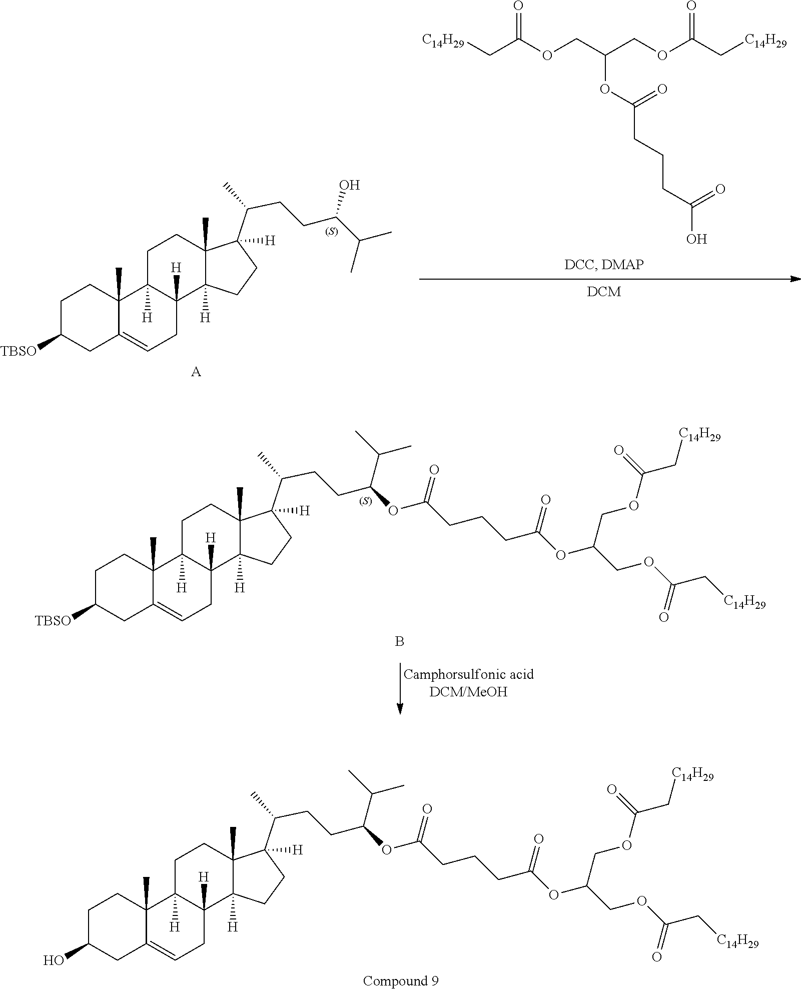

### Step 1: Synthesis of B

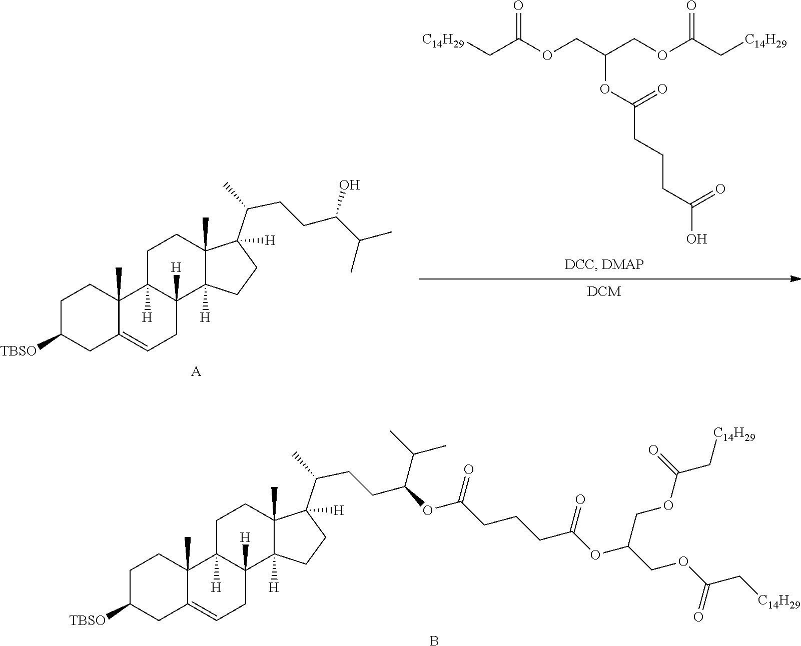

To a solution of A (1.00 g, 1.93 mmol) in DCM (10.0 mL) was added DMAP (236 mg, 1.93 mmol), DCC (798 mg, 3.87 mmol) and 5-((1,3-bis(palmitoyloxy)propan-2-yl)oxy)-5-oxopentanoic acid (1.98 g, 2.90 mmol) at 0° C. under N_{2(g)}. After stirring at 20° C. for 16 hours, the mixture was quenched with water (10 mL) and extracted with DCM (2×10 mL). The combined organic layers were washed with brine (2×10 mL), dried over anhydrous Na₂SO₄, filtered, and purified by column (0-6% EtOAc in petroleum ether) to give B (1.5 g, 66% yield. ¹H NMR (400 MHz, CDCl₃) δ_{H} 5.31-5.24 (m, 2H), 4.72-4.19 (m, 1H), 4.32-4.28 (m, 2H), 4.15-4.09 (m, 3H), 3.50-3.45 (m, 1H), 2.42-2.29 (m, 10H), 2.04-1.94 (m, 7H), 1.83-1.76 (m, 4H), 1.62-1.57 (m, 10H), 1.48-1.42 (m, 2H), 1.27-1.25 (m, 50H), 0.99 (s, 5H), 0.92-0.86 (m, 26H), 0.66 (s, 3H), 0.053 (s, 6H).

### Step 2: Synthesis of Compound 9

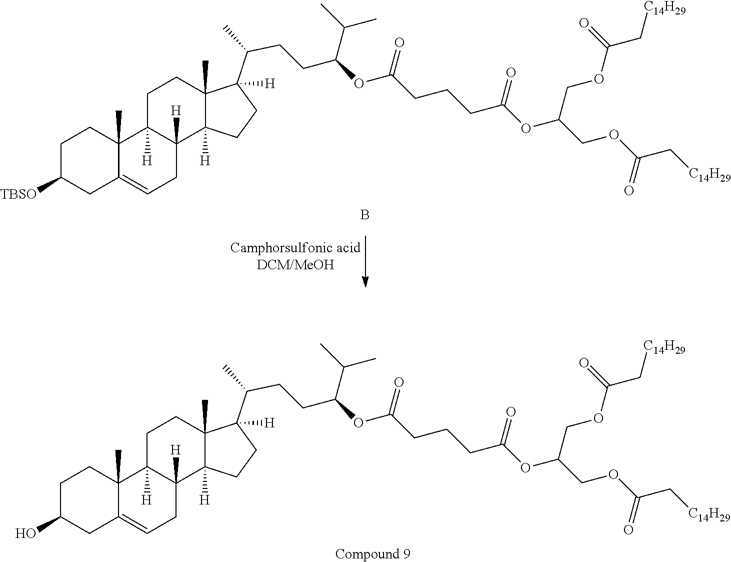

To a solution of B (1.20 g, 1.02 mmol) in DCM (10 mL) and MeOH (10 mL) was added Camphorsulfonic acid (70.8 mg, 0.305 mmol) at 0° C. under N_{2(g)}. After stirring for 1 hour, the mixture was quenched with water (5 mL) and extracted with DCM (2×5 mL). The combined organic layers were washed with brine (2×10 mL), dried over anhydrous Na₂SO₄, filtered and purified by SFC (Column: Column: Chiralpak IK-3 50×4.6 mm I.D., 3 um, Mobile phase: Phase A for CO₂, and Phase B for MeOH (0.05% DEA); Isocratic elution: 40% B in A, Flow rate: 3 mL/min; Detector: PDA; Column Temp: 35 C; Back Pressure: 100Bar) to give Compound 9 (505.6 mg, 42% yield). ¹H NMR (400 MHz, CDCl₃) δ_{H} 5.34-5.33 (m, 1H), 5.27-2.25 (m, 1H), 4.70-4.69 (m, 1H), 4.32-4.28 (m, 2H), 3.52-3.49 (m, 1H), 2.42-2.29 (m, 10H), 1.97-1.79 (m, 9H), 1.59-1.48 (m, 14H), 1.25 (s, 50H), 1.15-1.07 (m, 5H) 1.00 (s, 4H), 0.92-0.88 (m, 17H), 0.67 (s, 3H). ¹³C NMR (101 MHz, CDCl₃) δ_{C} 173.28, 172.64, 172.03, 140.77, 121.65, 79.18, 77.34, 77.03, 76.70, 71.77, 69.16, 62.03, 56.73, 50.09, 42.32, 36.49, 34.02, 31.93, 31.66, 29.71, 29.49, 29.37, 29.13, 24.85, 22.69, 20.25, 19.39, 18.79, 17.30, 14.12, 11.85. LC-ELSD/MS 50-100AB_6 min_ELSD_2000, purity >99%; MS ESI calcd for C₆₇H₁₁₈O₉Na [M+Na]⁺ 1089.9. found 1089.7.

### Example 3: Synthesis of Compound 19

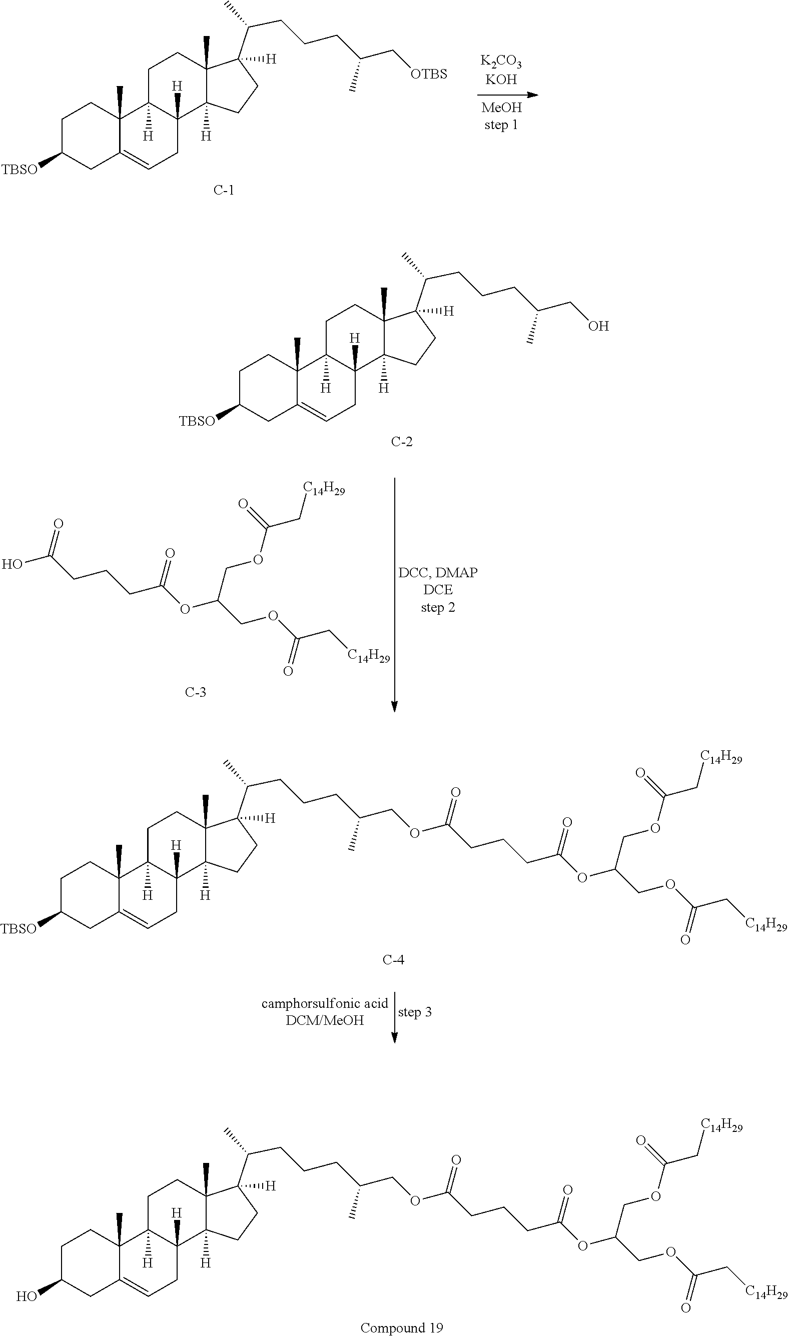

### Step 1: Synthesis of Compound C-2

To a solution of Compound C-1 (8.50 g, 13.5 mmol) in MeOH (80.0 mL) was added K₂CO₃ (3.72 g, 26.9 mmol) and KOH (1.51 g, 26.9 mmol) at 20° C. under N_{2(g)}. After stirring at 60° C. for 16 hours, the mixture was diluted with EtOAc (100 mL) and washed with H₂O (2×80 mL), dried over anhydrous Na₂SO₄, filtered, concentrated, and purified by recrystallization from ACN (100 mL) to give Compound C-2 (4.10 g, 58.9% yield). ¹H NMR (400 MHz, CDCl₃) δ_{H} 5.33-5.29 (m, 1H), 3.54-3.36 (m, 3H), 2.32-1.92 (m, 5H), 1.85-1.69 (m, 3H), 1.65-1.50 (m, 6H), 1.49-1.17 (m, 10H), 1.16-0.97 (m, 10H), 0.93-0.90 (m, 5H), 0.89-0.87 (m, 8H), 0.79-0.64 (m, 3H), 0.06 (s, 6H).

### Step 2: Synthesis of Compound C-4

To a solution of Compound C-3 (3.17 g, 4.64 mmol) in DCE (20 mL) was added DCC (1.20 g, 5.80 mmol) and DMAP (473 mg, 3.87 mmol) at 50° C. under N_{2(g)}. After stirring at 20° C. for 30 min, Compound C-2 (2.00 g, 3.87 mmol) was added to the mixture. After stirring at 50° C. for 16 hours, the mixture was filtered, concentrated, and purified by silica gel chromatography (0-4% EtOAc in petroleum ether) to give Compound C-4 (1.03 g, 22.5% yield). ¹H NMR (400 MHz, CDCl₃) δ_{H} 5.36-5.15 (m, 2H), 4.37-4.25 (m, 2H), 4.19-4.07 (m, 2H), 4.03-3.78 (m, 2H), 3.52-3.43 (m, 1H), 2.45-2.25 (m, 9H), 2.08-1.90 (m, 6H), 1.84-1.68 (m, 7H), 1.66-1.56 (m, 9H), 1.51-1.44 (m, 3H), 1.30-1.23 (m, 54H), 1.06-0.95 (m, 7H), 0.92-0.85 (m, 19H), 0.79-0.59 (m, 3H), 0.05 (s, 6H).

### Step 3: Synthesis of Compound 19

To a solution of Compound C-4 (2.00 g, 1.69 mmol) in a mixture of DCM (10 mL) and MeOH (10 mL) was added camphorsulfonic acid (118 mg, 0.508 mmol) at 0° C. under N_{2(g)}. After stirring at 0° C. for 1 hour, the mixture was quenched with water (50 mL) and extracted with DCM (3×20 mL). The combined organic layers were washed with Brine (20 mL), dried over anhydrous Na₂SO₄, filtered, concentrated, purified by silica gel chromatography (0%-10% of Ethyl Acetate in petroleum ether) and purified by SFC (Instrument: CAS-CD-Prep-SFC-F; Column: DAICEL CHIRALPAK AD (250 mm*30 mm, 10 um); Condition: CO₂-i-PrOH; Begin B:35; End B:35; Gradient Time (8.8 min); 100% B Hold Time (0 min); Flow Rate (70 mL/min); Injections: 266, Rt=1.622 min) to give Compound 19 (520.0 mg, 44.1% yield). ¹H NMR (400 MHz, CDCl₃) δ_{H} 5.38-5.32 (m, 1H), 5.30-5.18 (m, 1H), 4.34-4.26 (m, 2H), 4.19-4.09 (m, 2H), 4.02-3.91 (m, 1H), 3.91-3.79 (m, 1H), 3.59-3.45 (m, 1H), 2.44-2.35 (m, 4H), 2.34-2.20 (m, 6H), 2.06-1.91 (m, 4H), 1.89-1.76 (m, 4H), 1.63-1.57 (m, 5H), 1.50-1.42 (m, 5H), 1.25 (s, 54H), 1.13-0.98 (s, 12H), 0.93-0.85 (m, 12H), 0.68 (s, 3H). ¹³C NMR (101 MHz, CDCl₃) δ_{C} 173.28, 173.28, 172.91, 172.02, 140.77, 122.10, 121.69, 102.48, 71.81, 69.60, 69.20, 68.81, 62.02, 58.77, 56.11, 50.14, 42.34, 42.32, 39.79, 37.26, 36.51, 36.17, 36.06, 35.77, 35.72, 34.20, 34.03, 33.79, 33.23, 33.18, 33.04, 32.58, 31.93, 31.93, 31.80, 31.66, 29.70, 29.70, 29.70, 29.67, 29.67, 29.67, 29.63, 29.46, 29.36, 29.27, 29.12, 28.95, 28.25, 24.89, 24.85, 24.85, 24.29, 23.28, 22.69, 22.69, 22.60, 21.09, 20.08, 19.39, 19.34, 16.67, 16.83, 14.11, 14.11, 11.88, 1.10. LC-ELSD/MS purity >99%, MS ESI calcd. for C₂₇H₄₅ [M+H-C₄₀H₇₄O₉]⁺ 369.3. found 369.3.

### Example 4: Synthesis of Compound 194

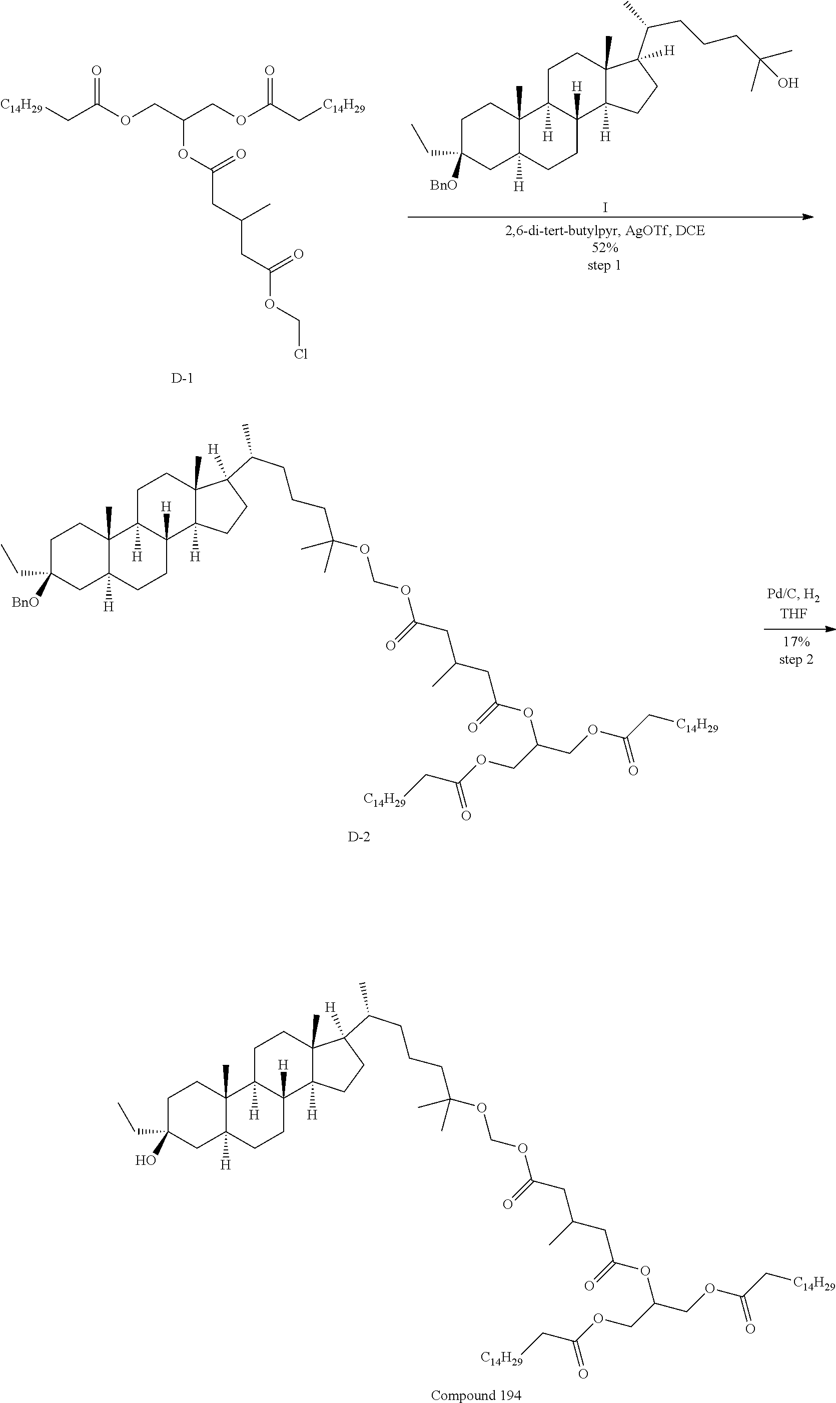

### Step 1: Synthesis of Compound D-2

To a solution of (R)-6-((3S,5S,8R,9S,10S,13R,14S,17R)-3-(benzyloxy)-3-ethyl-10,13-dimethylhexadecahydro-1H-cyclopenta[a]phenanthren-17-yl)-2-methylheptan-2-ol (I, 836 mg, 1.60 mmol) in DCE (18.0 mL) was added AgOTf (123 mg, 4.82 mmol) and 2,6-di-tert-butylpyridine (1.07 mg, 5.62 mmol) at 20° C. The reaction was cooled to 0° C. and Compound D-1 (1.8 g, 2.41 mmol) was added. After stirring at 40° C. for 16 hours, the mixture was quenched with water (50 mL) and extracted with DCM (3×50 mL). The combined organic phase was washed with brine (2×300 mL), dried over anhydrous Na₂SO₄, filtered, and purified by flash column (0-3% of EtOAc in petroleum ether) to give Compound D-2 (1.03 g, 52% yield). ¹H NMR (400 MHz, CDCl₃) δ_{H} 7.38-7.27 (m, 4H), 7.24-7.16 (m, 1H), 5.42-5.36 (m, 2H), 5.28-5.20 (m, 1H), 4.41 (s, 2H), 4.25-4.32 (m, 2H), 4.18-4.07 (m, 2H), 2.50-2.35 (m, 3H), 2.32-2.25 (m, 4H), 1.99-1.92 (m, 1H), 1.78-1.58 (m, 13H), 1.55-1.45 (m, 10H), 1.25 (s, 48H), 1.21 (s, 7H), 1.10-1.02 (m, 8H), 0.94-0.82 (m, 22H), 0.65 (s, 4H).

### Step 2: Synthesis of Compound 194

To a solution of Compound D-2 (1.5 g, 1.21 mmol) in THF (15 mL) was added Pd/C (800 mg, 10% Palladium on carbon, 1% water wet) under H_{2(g)}. After stirring at 40° C. for 16 hours, the mixture was filtered through a pad of celite and washed with THE (100 mL), filtered, concentrated, and purified by flash column (0-7% of EtOAc in petroleum ether) to give Compound 194 (716.1 mg, 17% yield). ¹H NMR (400 MHz, CDCl₃) δ_{H} 5.38-5.32 (m, 2H), 5.30-5.25 (m, 1H), 4.32-4.26 (m, 2H), 4.17-4.10 (m, 2H), 2.49-2.34 (m, 3H), 2.33-2.21 (m, 6H), 1.99-1.92 (m, 1H), 1.83-1.75 (m, 1H), 1.65-1.51 (m, 13H), 1.48-1.34 (m, 9H), 1.31-1.23 (m, 51H), 1.21 (s, 6H), 1.19-0.96 (m, 11H), 0.92-0.85 (m, 13H), 0.83 (s, 3H), 0.65 (s, 4H). QNMR (1,2,4,5-tetrachloro-3-nitrobenzene, 99.8% as IS) 95.68% wt found. ¹³C NMR (101 MHz, CDCl₃) Sc 173.26, 171.66, 171.31, 84.17, 78.13, 72.80, 69.06, 62.04, 56.50, 56.25, 54.54, 43.66, 42.61, 40.01, 35.90, 35.76, 35.49, 33.98, 31.90, 29.68, 29.46, 29.34, 29.24, 29.10, 26.12, 24.81, 22.66, 19.59, 14.10, 12.06, 7.08.

### Example 5: Synthesis of Compound 121

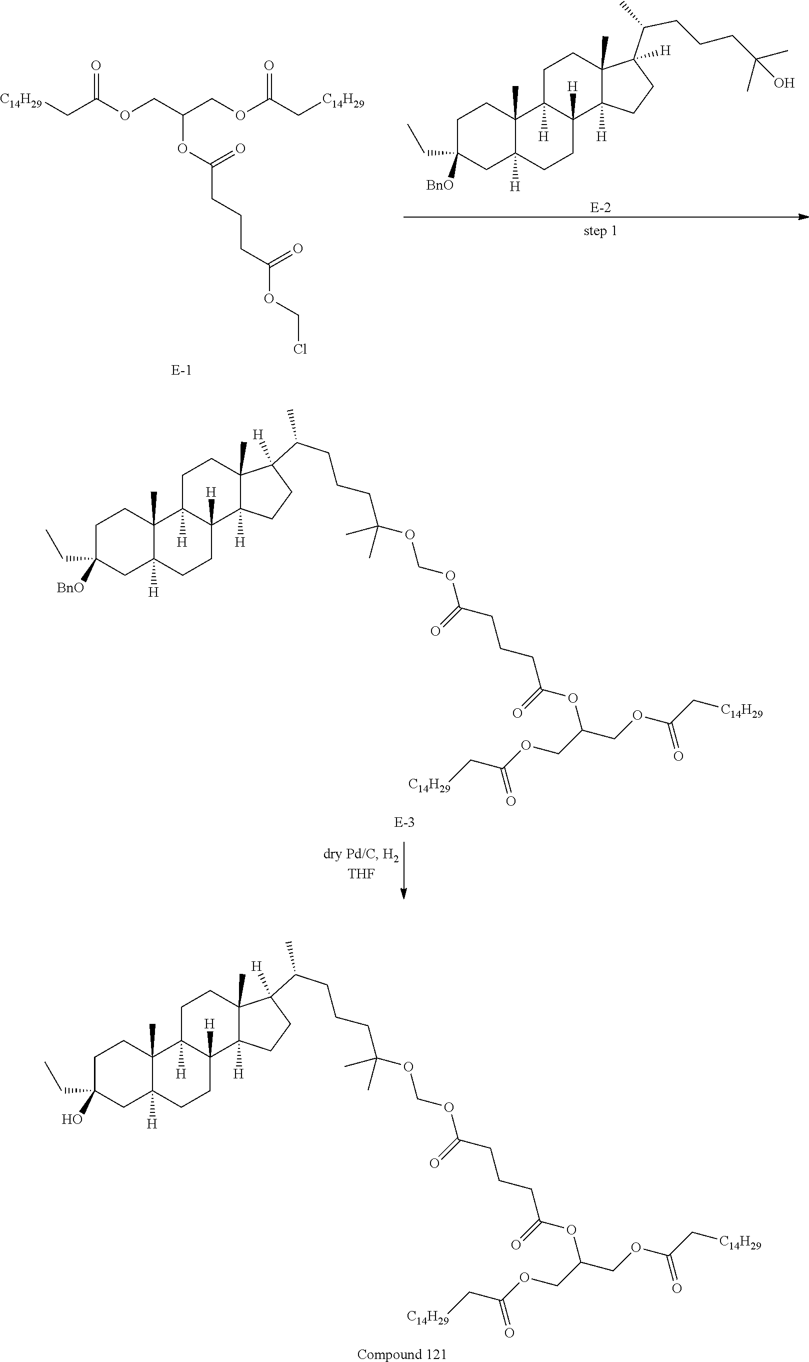

### Step 1. Synthesis of Compound E-3

Two batches were prepared as follows. To a solution of Compound E-2 (1.00 g, 1.91 mmol) in DCE (10 mL) was added AgOTf (1.46 g, 5.72 mmol) and 2,6-di-tert-butylpyridine (1.27 g, 6.68 mmol) at 20° C. Compound E-1 (2.79 g, 3.82 mmol) was added to the reaction mixture at 0° C. After stirring at 40° C. for 16 h the reaction was quenched with H₂O (20 mL) and extracted with DCM (2×50 mL). The combined organic phase was washed with saturated brine (2×40 mL). The organic layers from both batches were combined, dried over anhydrous Na₂SO₄, filtered, concentrated, and purified by flash column (0-5% of EtOAc in petroleum ether) to give Compound E-3 (3.0 g, 65% yield). 1H NMR (400 MHz, CDCl₃) δ_{H} 7.39-7.27 (m, 4H), 7.26-7.19 (m, 1H), 5.40-5.33 (m, 2H), 5.29-5.22 (m, 1H), 4.48-4.34 (m, 2H), 4.34-4.27 (m, 2H), 4.17-4.09 (m, 2H), 2.43-2.25 (m, 4H), 2.33-2.28 (m, 4H), 2.00-1.91 (m, 3H), 1.75-1.59 (m, 10H), 1.57-1.41 (m, 10H), 1.36-1.23 (m, 58H), 1.23-1.16 (m, 8H), 0.98-0.80 (m, 17H), 0.70-0.58 (m, 4H).

### Step 2. Synthesis of Compound 121

A mixture of Compound E-3 (3.0 g, 2.46 mmol) and Pd/C (1.1 g, 10% Palladium on carbon) in THE (150 mL) was hydrogenated under 25 psi of hydrogen at 25° C. for 16 hours. The reaction mixture was filtered through celite, washed with THE (100 mL), concentrated, and purified by flash column (0-3% of EtOAc in petroleum ether) to give Compound 121 (953.7 mg, 31% yield). 1H NMR (400 MHz, CDCl₃) δ_{H} 5.45-5.30 (m, 2H), 5.29-5.22 (m, 1H), 4.34-4.24 (m, 2H), 4.19-4.07 (m, 2H), 2.44-2.34 (m, 4H), 2.34-2.27 (m, 4H), 2.02-1.90 (m, 3H), 1.58-1.53 (m, 12H), 1.46-1.33 (m, 9H), 1.31-1.23 (m, 50H), 1.22-1.19 (m, 8H), 1.12-0.94 (m, 8H), 0.92-0.79 (m, 17H), 0.67-0.61 (m, 4H). ¹³C NMR (101 MHz, CDCl₃) δC=173.28, 171.96, 84.25, 78.15, 77.35, 72.80, 69.20, 62.00, 56.53, 54.57, 43.70, 40.05, 35.53, 34.01, 33.13, 31.93, 29.70, 29.12, 24.84, 24.19, 22.69, 19.81, 14.11, 12.10, 7.10.

### Example 6: Synthesis of Compound 80

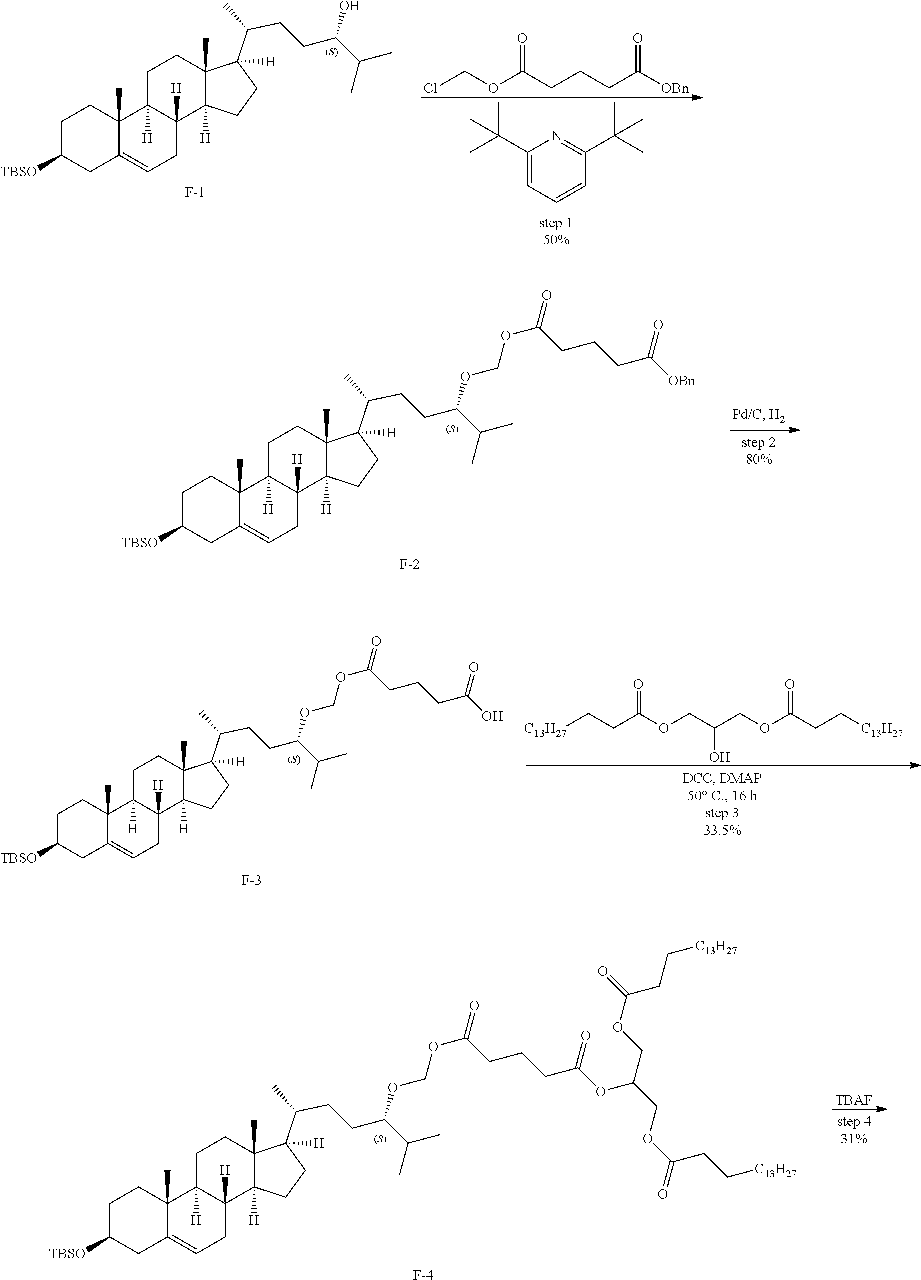

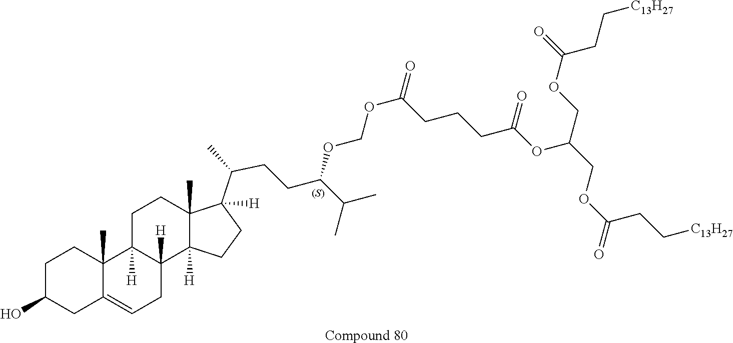

### Step 1: Synthesis of Compound F-2

Two batches were prepared as follows. To a solution of Compound F-1 (2.50 g, 4.84 mmol) in DCM (25.0 mL) was added AgOTf (3.73 g, 14.5 mmol) and 2,6-Di-tert-butylpyridine (3.24 g, 3.79 mL, 16.9 mmol) at 25° C. under N₂. Benzyl (chloromethyl) glutarate (3.93 g, 14.5 mmol) was added at 0° C. and the reaction was stirred at 25° C. for 30 min. Both batches were combined and quenched with NH₄Cl_{(aq)} (100 mL) and extracted with EtOAc (2×100 mL). The combined organic layers were washed with brine (100 mL), dried over anhydrous Na₂SO₄, filtered, concentrated and purified by column (0-3% EtOAc in petroleum ether) to give Compound F-2 (4.30 g, 50% yield). ¹H NMR (400 MHz, CDCl₃) δ_{H} 7.38-7.31 (m. 5H), 5.37-5.26 (m, 3H), 5.11 (s, 1H), 3.52-3.43 (m, 1H), 3.32-3.25 (m, 1H), 2.45-2.35 (m, 3H), 2.30-2.11 (m, 2H), 2.02-1.94 (m, 4H), 1.84-1.64 (m. 6H), 1.25-0.98 (m, 14H), 0.95-0.85 (m, 25H), 0.70-0.64 (m, 4H), 0.06 (s, 6H).

### Step 2: Synthesis of Compound F-3

To a solution of Compound F-2 (5.00 g, 6.66 mmol) in THF (50.0 mL) was added wet 10% Pd/C (500 mg, 4.7 mmol) under Ar_{(g)}. The suspension was degassed under vacuum and purged with H_{2(g)} three times. The mixture was stirred under H_{2(g)} (15 psi) at 25° C. for 2 hours to give a black suspension. The reaction mixture was filtered through a pad of celite and washed with THE (100 mL). The filtrate was concentrated to give Compound F-3 (4.30 g, 80%).

### Step 3: Synthesis of Compound F-4

To a solution of 2-hydroxypropane-1,3-diyl dipalmitate (4.54 g, 7.99 mmol) in DCE (45.0 mL) was added DCC (2.06 g, 9.98 mmol) and DMAP (813 mg, 6.66 mmol) at 25° C. under N_{2(g)}. Compound F-3 (4.30 g, 6.66 mmol) was added and the reaction was stirred at 50° C. for 16 h. The mixture was quenched with water (200 mL) and extracted with DCM (2×200 mL). The combined organic layers were washed with water (200 mL), dried over anhydrous Na₂SO₄, filtered, concentrated and purified by silica gel chromatography (0%-3% of Ethyl Acetate in Petroleum Ether) to give Compound F-4 (2.70 g, 33.5%). ¹H NMR (400 MHz, CDCl₃) δ_{H} 5.33-5.25 (m, 4H), 4.33-4.48 (m, 2H), 4.15-4.11 (m, 2H), 3.53-3.42 (m, 1H), 3.30-3.25 (m, 1H), 2.43-2.38 (m, 5H), 2.33-2.28 (m, 5H), 2.05-1.92 (m, 5H), 1.83-1.63 (m, 5H), 1.32-1.23 (m, 65H), 0.99 (s, 5H), 0.90-0.85 (m, 25H), 0.67 (s, 3H), 0.06 (s, 6H).

### Step 4: Synthesis of Compound 80

To a solution of Compound F-4 (2.7 g, 2.2 mmol) in THE (20.0 mL) was added TBAF (4.5 mL, 1.00 M in THF, 4.5 mmol) at 25° C. under N_{2(g)} and the reaction was stirred at 25° C. for 16 hours. The mixture was quenched with NH₄Cl_{(aq)} (50 mL) and extracted with EtOAc (2×50 mL). The combined organic layers were washed with water (50 mL), dried over anhydrous Na₂SO₄, filtered, and concentrated. The residue was purified by silica gel chromatography (0%-13% EtOAc in petroleum ether) to give Compound 80 (766.7 mg, 0.6985 mmol, 31% yield). ¹H NMR (400 MHz, CDCl₃) δ_{H} 5.37-5.22 (m, 4H), 4.34-4.26 (m, 2H), 4.16-4.10 (m, 2H), 3.58-3.47 (m, 1H), 3.31-3.24 (m, 1H), 2.42-2.38 (m, 4H), 2.32-2.22 (m, 6H), 2.03-1.93 (m, 4H), 1.86-1.73 (m, 4H), 1.62-1.58 (m, 5H), 1.50-1.43 (m, 4H), 1.34-1.21 (m, 54H), 1.18-1.05 (m, 5H), 1.01 (s, 4H), 0.94-0.84 (m, 17H), 0.68 (s, 3H). ¹³C NMR (101 MHz, CDCl₃) δ_{C} 173.275, 172.407, 171.942, 140.745, 121.653, 88.803, 86.026, 71.775, 69.189, 61.982, 56.729, 55.880, 50.088, 42.323, 39.756, 36.486, 34.010, 33.115, 31.918, 29.689, 29.653, 29.479, 29.351, 29.260, 29.114, 24.838, 22.682, 19.375, 18.745, 18.416, 14.114, 11.857. LC-ELSD/MS LIPID_50-100AB_6 min_ELSD_2000.M (100.0% purity), MS ESI calcd for C₆₈H₁₂₀O₁₀Na [M+Na]+1119.88 not found, detected fragment calcd for C₃₅H₆₇O₄[M-C₃₅H₆₇O₄]⁺ 551.5. found 551.5.

### Example 7: Synthesis of Compound 293

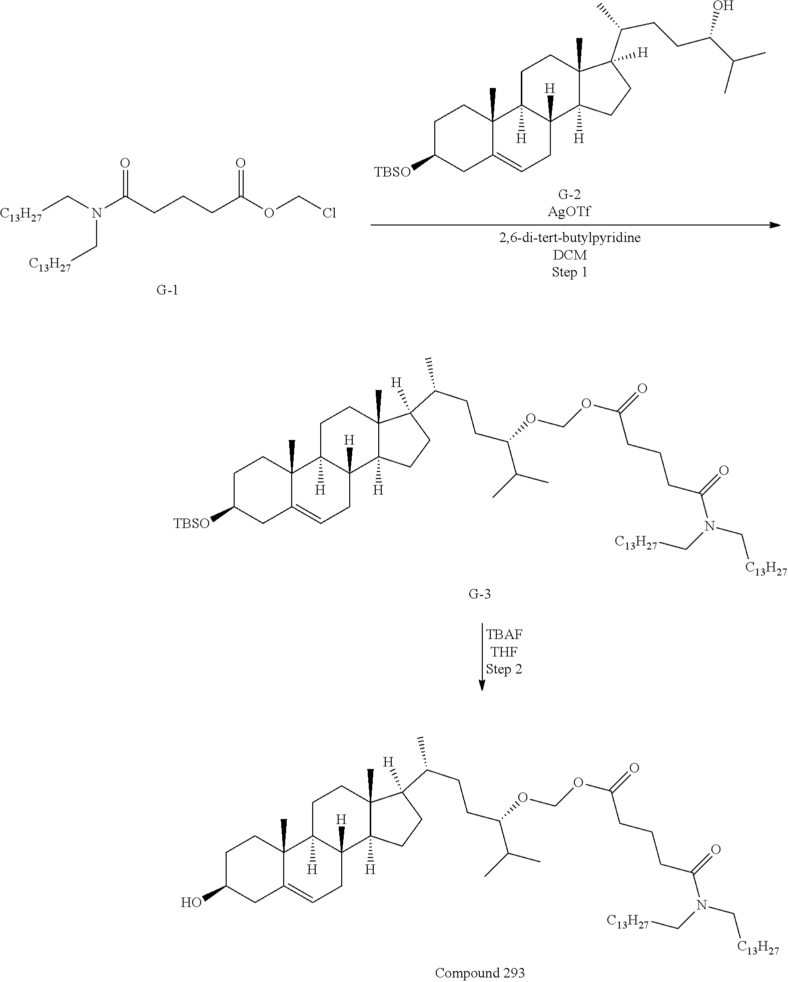

### Step 1: Synthesis of Compound G-3

To a solution of Compound G-2 (1.00 g, 1.93 mmol) in DCM (10.0 mL) was added AgOTf (1.49 g, 5.80 mmol) and 2,6-di-tert-butylpyridine (1.30 g, 1.52 mL, 6.77 mmol) at 25° C. under N_{2(g)}. The mixture was then cooled to 0° C. and Compound G-1 (1.11 g, 1.93 mmol) was added. The reaction mixture was warmed to 25° C. and stirred for 16 hours, then quenched with sat. NH₄Cl_{(aq)} (20 mL) and extracted with DCM (2×20 mL). The combined organic layers were washed with brine (30 mL), dried over anhydrous Na₂SO₄, filtered, concentrated and the residue purified by flash chromatography (0-10% EtOAc in petroleum ether) to afford Compound G-3 (700 mg, 34.4% yield). ¹H NMR (400 MHz, CDCl₃) δ_{H} 5.35-5.21 (m, 2H), 3.55-3.41 (m, 1H), 3.32-3.24 (m, 3H), 3.22-3.15 (m, 2H), 2.47-2.33 (m, 4H), 2.30-2.11 (m, 2H), 2.06-1.94 (m, 4H), 1.87-1.67 (m, 4H), 1.54-1.39 (m, 10H), 1.34-1.26 (m, 50H), 1.19-1.03 (m, 5H), 1.00 (s, 3H), 0.92 (d, J=6.4 Hz, 3H), 0.90-0.84 (m, 23H), 0.68-0.63 (m, 3H), 0.06 (s, 6H).

### Step 2: Synthesis of Compound 293

To a solution of Compound G-3 (700 mg, 665 mol) in THE (30.0 mL) was added TBAF (87.3 mg, 2.66 mL, 1.00 molar, 2.66 mmol) at 25° C. under N_{2(g)}. The reaction mixture was stirred at 25° C. for 4 h, then quenched with sat. NH₄Cl_{(aq)} (5 mL) and extracted with EtOAc (2×20 mL). The combined organic layers were washed with water, dried over anhydrous Na₂SO₄, filtered, concentrated, and the residue purified twice by silica gel chromatography (0-15% EtOAc in petroleum ether) and purified by SFC (Column: DAICEL CHIRALPAK AD (250 mm×30 mm, 10 μm); Condition:CO₂-EtOH (0.1% NH₃H₂O); Begin B:35; End B:35; Gradient Time (45 min); 100% B Hold Time (0 min); Flow Rate (80 mL/min); Injections:60) to afford Compound 293 (222 mg, 35.5% yield). ¹H NMR (400 MHz, CDCl₃) δ=5.36-5.32 (m, 1H), 5.32-5.23 (m, 2H), 3.58-3.47 (m, 1H), 3.32-3.24 (m, 3H), 3.23-3.14 (m, 2H), 2.49-2.34 (m, 4H), 2.33-2.16 (m, 2H), 2.07-1.91 (m, 4H), 1.89-1.74 (m, 4H), 1.56-1.33 (m, 15H), 1.31-1.21 (m, 46H), 1.17-1.03 (m, 5H), 1.01 (s, 3H), 0.99-0.96 (m, 1H), 0.92 (d, J=6.4 Hz, 3H), 0.90-0.84 (m, 12H), 0.67 (s, 3H). LC-ELSD/MS ESI calcd. for C₆₁H₁₁₂NO₅ [M+H]⁺ 938.8 found 938.8.

### Example 7: Synthesis of Compound 334

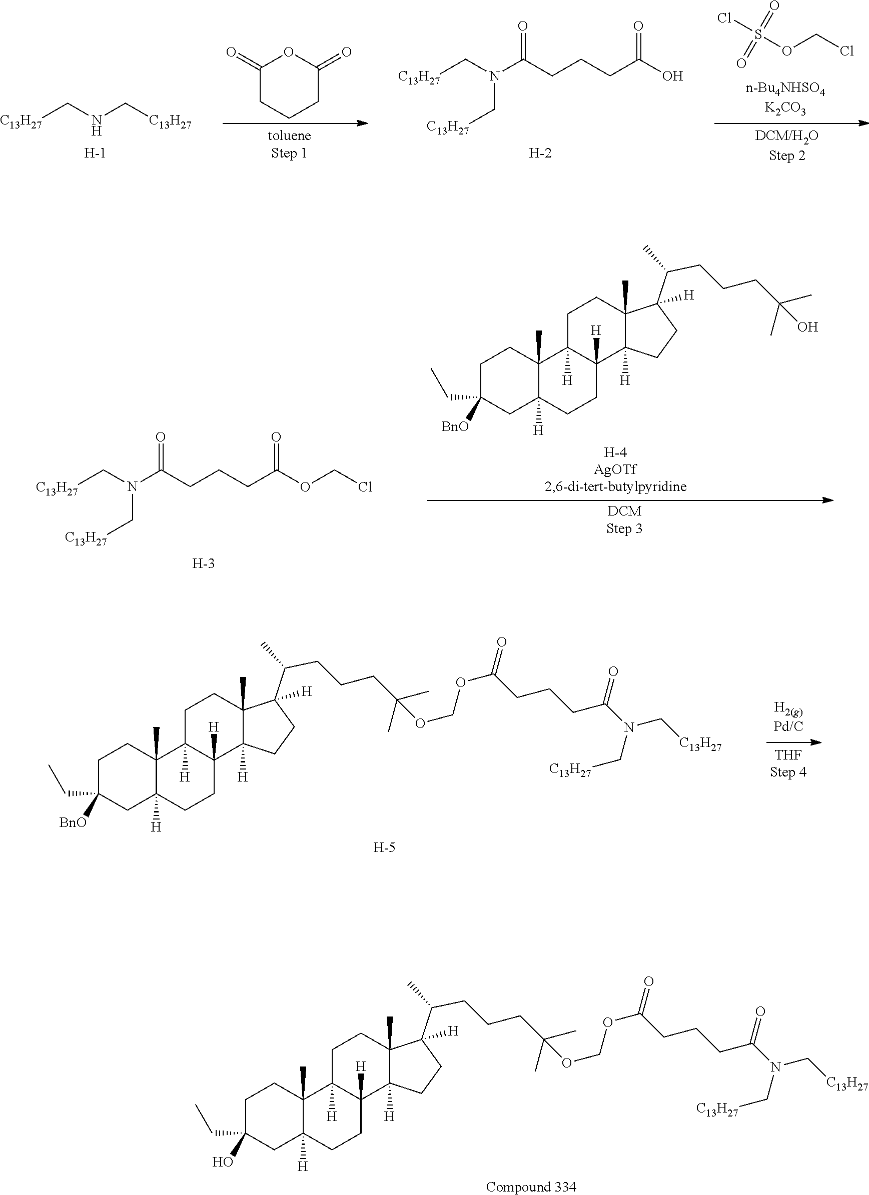

### Step 1: Synthesis of Compound H-2

To a solution of dihydro-2H-pyran-2,6(3H)-dione (2.78 g, 24.4 mmol) in toluene (80 mL) was added Compound H-1 (10.0 g, 24.4 mmol) under N_{2(g)}. The mixture was heated to 85° C. for 3 h, then the mixture was concentrated to afford Compound H-2 (12 g, 94% yield). ¹H NMR (400 MHz, CDCl₃) δ_{H} 3.35-3.26 (m, 2H), 3.24-3.13 (m, 2H), 2.45 (q, J=6.8 Hz, 4H), 2.03-1.92 (m, 2H), 1.59-1.46 (m, 4H), 1.37-1.17 (m, 45H), 0.88 (t, J=6.8 Hz, 6H).

### Step 2: Synthesis of Compound H-3

To a solution of Compound H-2 (12.0 g, 22.9 mmol), tetra(but-1-yl)ammonium bisulfate (778 mg, 2.29 mmol) in DCM (120 mL) was added K₂CO₃ (12.7 g, 91.6 mmol) in H₂O (120 mL) under N_{2(g)}. The mixture was stirred at 25° C. for 0.1 h, then a solution of chloromethyl chlorosulfate (5.67 g, 34.4 mmol) in DCM (120 mL) was added dropwise. The mixture was stirred at 25° C. for 16 h, then DCM (100 mL) and brine (100 mL) were added to the mixture. The organic layer was separated, concentrated, and the residue was purified by flash chromatography (0-20% EtOAc in petroleum ether) to afford Compound H-3 (6 g, 46% yield). ¹H NMR (400 MHz, CDCl₃) δ_{H} 5.70 (s, 2H), 3.33-3.24 (m, 2H), 3.22-3.10 (m, 2H), 2.49 (t, J=7.2 Hz, 2H), 2.37 (t, J=7.2 Hz, 2H), 2.11-1.91 (m, 2H), 1.58-1.44 (m, 4H), 1.29-1.21 (m, 44H), 0.88 (t, J=6.8 Hz, 6H).

### Step 3: Synthesis of Compound H-5

To a solution of Compound H-4 (2.00 g, 3.83 mmol) in DCM (20.0 mL) was added AgOTf (2.95 g, 11.5 mmol) and 2,6-di-tert-butylpyridine (2.56 g, 3.00 mL, 13.4 mmol) at 25° C. under N_{2(g)}. The mixture was cooled to 0° C., and Compound H-3 (2.19 g, 3.83 mmol) was added. The mixture was warmed to 25° C. and stirred for 16 hours, then quenched with sat. NH₄Cl_{(aq)} (20 mL) and extracted with EtOAc (2×20 mL). The combined organic layers were washed with brine (20 mL), dried over anhydrous Na₂SO₄, filtered, concentrated and the residue purified by flash chromatography (0-10% EtOAc in petroleum ether) to afford Compound H-5 (1.50 g), which was triturated with MeOH (50.0 mL) at 25° C. for 1 hour. The mixture was filtered and concentrated to afford Compound H-5 (800 mg, 21.6% yield). ¹H NMR (400 MHz, CDCl₃) δ_{H} 7.39-7.27 (m, 5H), 5.35 (s, 2H), 4.41 (s, 2H), 3.30-3.24 (m, 2H), 3.22-3.15 (m, 2H), 2.43-2.32 (m, 4H), 2.02-1.87 (m, 4H), 1.83-1.59 (m, 9H), 1.54-1.43 (m, 9H), 1.33-1.24 (m, 58H), 1.13-1.04 m, 4H), 0.93-0.85 (m, 15H), 0.83 (s, 3H), 0.65 (s, 4H).

### Step 4: Synthesis of Compound 334

To a solution of Compound H-5 (700 mg, 661 mol) in THE (15.0 mL) was added Pd/C (700 mg, 6.58 mmol) at 25° C. under N_{2(g)}. The mixture was then stirred at 25° C. under 15 psi H_{2(g)} for 24 h, then the reaction mixture was filtered, concentrated and the residue purified twice by flash chromatography (0-15% EtOAc in petroleum ether) to afford Compound 334 (221.7 mg, 30.2% yield). 1H NMR (400 MHz, CDCl₃) δ_{H} 5.35 (s, 2H), 3.32-3.24 (m, 2H), 3.23-3.11 (m, 2H), 2.46-2.28 (m, 4H), 2.03-1.91 (m, 3H), 1.86-1.74 (m, 1H), 1.68-1.36 (m, 22H), 1.30-1.25 (m, 44H), 1.20 (s, 8H), 1.17-0.93 (m, 9H), 0.92-0.85 (m, 13H), 0.82 (s, 3H), 0.64 (s, 4H). LC-ELSD/MS ESI calcd. for C₆₃H₁₁₈NO₅ [M+H]⁺ 968.8 found 968.8.

### Example 7: Synthesis of Compound 222

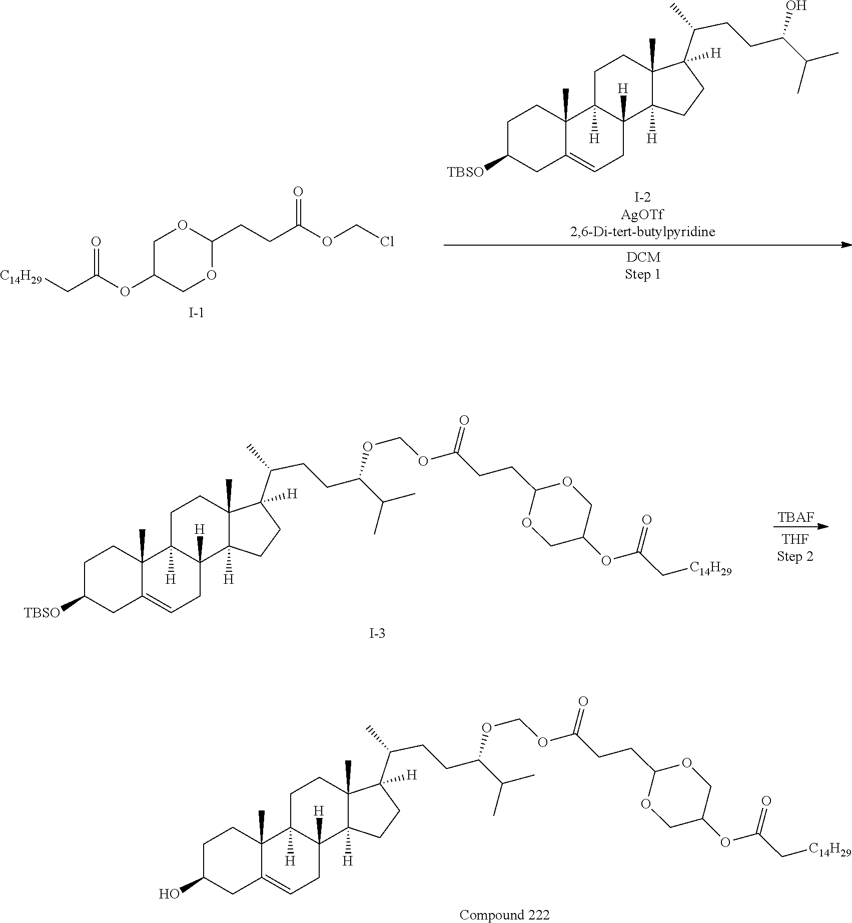

### Step 1: Synthesis of Compound I-3

To a solution of Compound I-2 (1.00 g, 1.93 mmol) in DCM (2.00 mL) was added Silver(I) Trifluoromethanesulfonate (1.49 g, 5.80 mmol) and 2,6-di-tert-butylpyridine (1.30 g, 6.77 mmol) at 25° C. under N_{2(g)}, then the reaction mixture was cooled to 0° C., and 2-(3-(chloromethoxy)-3-oxopropyl)-1,3-dioxan-5-yl palmitate I-1 (985 mg, 2.13 mmol) was added. The reaction mixture was allowed to warm to 25° C. and stirred for 16 h. The reaction mixture was directly purified by flash chromatography (0-6% EtOAc in petroleum ether) to afford Compound I-3 (960 mg, 53% yield). ¹H NMR (400 MHz, CDCl₃) 5.29 (s, 2H), 5.11-4.99 (m, 1H), 4.34-4.22 (m, 1H), 4.13-3.56 (m, 4H), 3.53-3.43 (m, 1H), 3.29-3.27 (m, 1H), 2.46 (q, J=8.0 Hz, 2H), 2.33 (t, J=7.6 Hz, 2H), 2.28-1.59 (m, 14H), 1.47-1.22 (m, 33H), 1.09-0.81 (m, 32H), 0.67 (s, 3H), 0.05 (s, 6H).

### Step 2: Synthesis of Compound 222

To a solution of Compound I-3 (832 mg, 0.882 mmol) in THF (4.00 mL) was added TBAF (692 mg, 2.65 mmol) at 25° C. under N_{2(g)}. After stirring at 25° C. for 16 h, the reaction mixture was quenched with water (30 mL), extracted with EtOAc (2×50 mL), and the combined organic layers were washed with brine (2×50 mL), dried over anhydrous Na₂SO₄, filtered, and concentrated. The residue was purified by flash chromatography (0-12% EtOAc in petroleum ether) and SFC (Column: DAICEL CHIRALCEL OD (250 mm×30 mm, 10 μm); Condition: CO₂-MeOH; Begin B: 50 End B: 50; Gradient Time (min): 7; Flowrate (ml/min): 70; Injections: 140) to afford Compound 222 (200.6 mg, 27% yield). ¹H NMR (400 MHz, CDCl₃) 5.36-5.25 (m, 1H), 5.29 (s, 2H), 5.12-4.97 (m, 1H), 4.27-3.53 (m, 5H), 3.32-3.23 (m, 1H), 2.46 (q, J=8.0 Hz, 2H), 2.34 (t, J=7.6 Hz, 2H), 2.29-1.76 (m, 10H), 1.67-1.46 (m, 12H), 1.34-1.22 (m, 26H), 1.18-0.84 (m, 23H), 0.68 (s, 3H). LC-ELSD/MS MS ESI calcd. for C₂₉H₄₉ [M+H-C₂₂H₄₀O₈]⁺ 397.4. found 397.4.

### Example 7: Synthesis of Compound 263

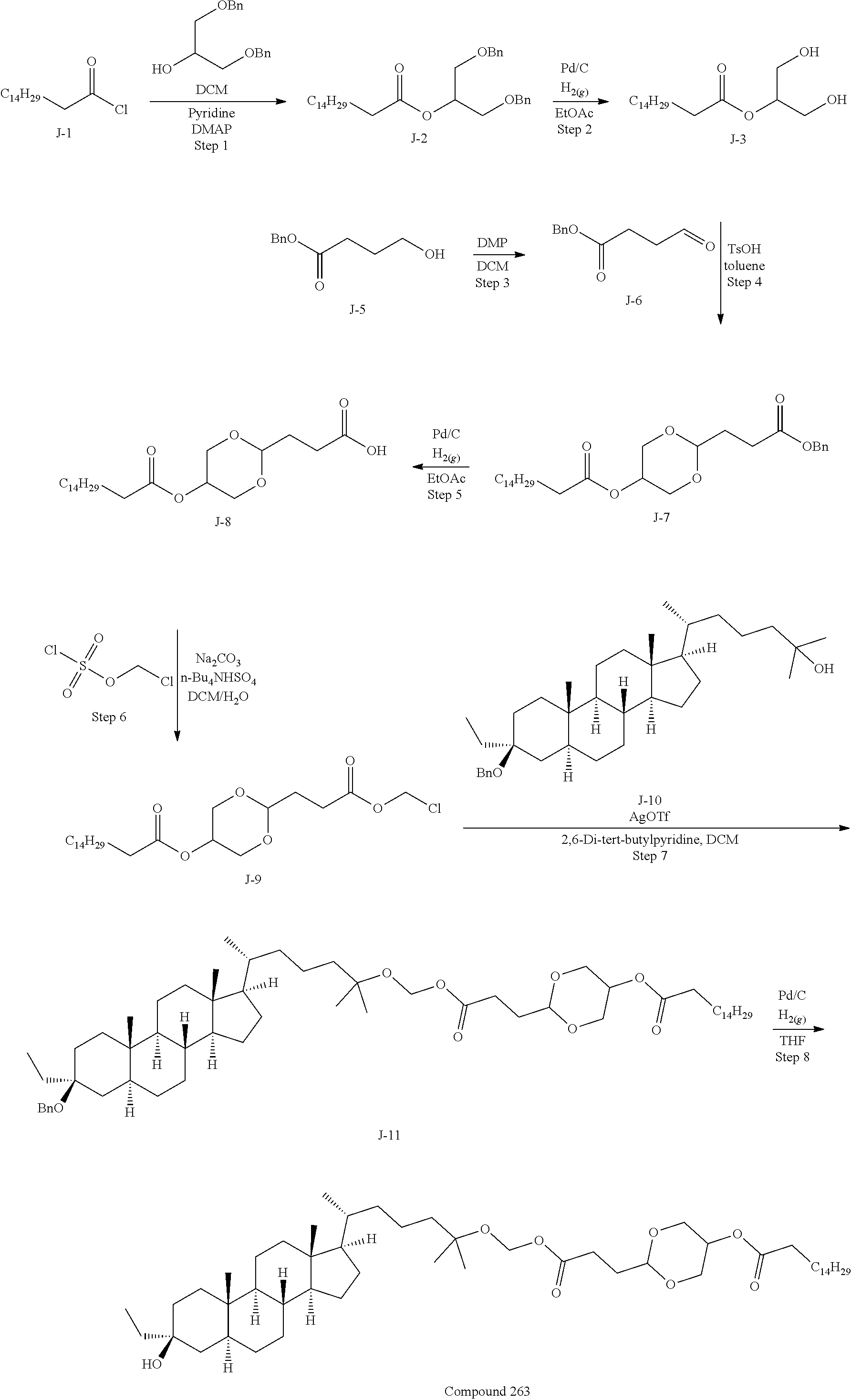

### Step 1: Synthesis of Compound J-2

To a solution of 1,3-bis(benzyloxy)propan-2-ol (6.00 g, 22.0 mmol) in DCM (150 mL) was added pyridine (8.71 g, 110 mmol), palmitoyl chloride Compound J-1 (15.1 g, 55.1 mmol) and DMAP (135 mg, 1.10 mmol) at 25° C. under N_{2(g)}. After stirring at 25° C. for 2 h, the reaction mixture was quenched with sat. NaHCO_{3(aq)} (200 mL), extracted with DCM (3×200 mL), the combined organic layers were dried over anhydrous Na₂SO₄, filtered, and concentrated. The residue was purified by flash chromatography (0-6% EtOAc in petroleum ether) to afford Compound J-2 (11.8 g, 98% yield). ¹H NMR (400 MHz, CDCl₃) 7.36-7.26 (m, 10H), 5.26-5.19 (m, 1H), 4.59-4.48 (m, 4H), 3.66-3.55 (m, 4H), 2.3-2.31 (m, 2H), 1.64-1.60 (m, 2H), 1.26-1.24 (m, 24H), 0.90-0.86 (m, 3H).

### Step 2: Synthesis of Compound J-3

To a solution of Compound J-2 (10.0 g, 19.6 mmol) in EtOAc (150 mL) was added wet Pd/C (1.04 g, 0.979 mmol) under N_{2(g)}. The reaction mixture was then stirred at 45° C. under 30 psi H_{2(g)} for 16 h, then filtered and concentrated to afford Compound J-3 (8 g) which was used directly without purification.

### Step 3: Synthesis of Compound J-6

To a solution of benzyl 4-hydroxybutanoate Compound J-5 (4.00 g, 20.6 mmol) in DCM (40.0) was added DMP (17.5 g, 41.2 mmol) at 25° C. under N_{2(g)}. After stirring at 25° C. for 2 h, the reaction mixture was quenched with 400 mL of a 1:1 mixture of saturated aqueous NaHCO₃ and Na₂S₂O₃ solutions, extracted with DCM (2×100 mL) and the combined organic layers concentrated to afford Compound J-6 (4 g) which was used directly without purification.

### Step 4: Synthesis of Compound J-7

To a solution of Compound J-3 (8.00 g, 24.2 mmol) in toluene (80.0 mL) was added Compound J-6 (3.88 g, 20.2 mmol) and p-toluenesulfonic acid monohydrate (76.7 mg, 0.403 mmol) at 25° C. under N_{2(g)}. The reaction mixture was heated to 110° C. and stirred for 2 h, then quenched with water (20 mL), extracted with EtOAc (2×200 mL), the combined organic layers dried over anhydrous Na₂SO₄, filtered, and concentrated. The residue was purified by flash chromatography (0-10% EtOAc in petroleum ether) to afford Compound J-7 (5.1 g, 50% yield over three steps). ¹H NMR (400 MHz, CDCl₃) 7.39-7.26 (m, 5H), 5.17-5.10 (m, 2H), 5.09-4.98 (m, 1H), 4.26-3.54 (m, 5H), 2.49 (q, J=8.0 Hz, 2H), 2.35-2.30 (m, 2H), 2.10-2.00 (m, 2H), 1.64-1.59 (m, 2H), 1.28-1.23 (m, 24H), 0.88 (t, J=6.8 Hz, 3H).

### Step 5: Synthesis of Compound J-8

To a solution of Compound J-7 (5.10 g, 10.1 mmol) in EtOAc (5.00 mL) was added wet Pd/C (1.08 g, 1.01 mmol) at 25° C. under N_{2(g)}. The mixture was then stirred at 45° C. under 30 psi H_{2(g)} for 16 h, then filtered and concentrated to afford Compound J-8 (4.1 g), which was used directly without purification.

### Step 6: Synthesis of Compound J-9

To a mixture of Compound J-8 (4.10 g, 9.89 mmol) in 1:1 DCM:H₂O (4.00 mL) at 0° C. was added sodium carbonate (4.19 g, 39.6 mmol) and chloromethyl chlorosulfonate (1.96 g, 11.9 mmol) under N_{2(g)}. The reaction mixture was stirred at 0° C. for 15 min, then tetra(but-1-yl)ammonium bisulphate (336 mg, 0.989 mmol) was added. The mixture was then stirred at 25° C. for 1 h, then quenched with water (40 mL), extracted with DCM (2×70 mL) and the combined organic layers concentrated. The residue was purified by flash chromatography (0-7% EtOAc in petroleum ether) to afford Compound J-9 (3.21 g, 70% yield via two steps). ¹H NMR (400 MHz, CDCl₃) 5.74-5.67 (m, 2H), 5.13-4.99 (m, 1H), 4.31-3.63 (m, 5H), 2.56-2.49 (m, 2H), 2.36-2.31 (m, 2H), 2.13-2.03 (m, 2H), 1.66-1.59 (m, 2H), 1.30-1.24 (m, 24H), 0.88 (t, J=6.8 Hz, 3H).

### Step 7: Synthesis of Compound J-11

To a solution of Compound J-10 (400 mg, 0.765 mmol) in DCM (5.00 mL) was added silver(I) trifluoromethanesulfonate (590 mg, 2.30 mmol) and 2,6-Di-tert-butylpyridine (512 mg, 2.68 mmol) at 25° C. under N_{2(g)}. The mixture was cooled to 0° C., and Compound J-9 (425 mg, 0.918 mmol) was added. The reaction mixture was warmed to 25° C. and stirred for 16 h, then was quenched with saturated NH₄Cl_{(aq)} (20 mL) and extracted with DCM (2×50 mL). The combined organic layers were washed with brine (50 mL), dried over anhydrous Na₂SO₄, filtered, and concentrated. The residue was purified by flash chromatography (0-6% EtOAc in petroleum ether) to afford Compound J-11 (430 mg, 52% yield). ¹H NMR (400 MHz, CDCl₃) 7.36-7.26 (m, 5H), 5.36 (s, 2H), 5.12-4.98 (m, 1H), 4.41 (s, 2H), 4.34-3.58 (m, 7H), 2.44 (q, J=7.6 Hz, 2H), 2.34 (t, J=7.6 Hz, 2H), 2.07-1.82 (m, 4H), 1.74-1.39 (m, 19H), 1.20-1.29 (m, 33H), 1.14-0.99 (m, 6H), 0.92-0.86 (m, 10H), 0.83 (s, 3H), 0.60-0.70 (m, 4H).

### Step 8: Synthesis of Compound 263

To a solution of Compound J-11 (430 mg, 0.464 mmol) in THF (7.00 mL) was added wet Pd/C (300 mg, 2.82 mmol) at 25° C. under N_{2(g)}. The mixture was then stirred at 25° C. under 15 psi H_{2(g)} for 16 h, then the reaction mixture was filtered and concentrated. The residue was purified by flash chromatography (0-16% EtOAc in petroleum ether) and lyophilized to afford Compound 263 (214.3 mg, 57%). ¹H NMR (400 MHz, CDCl₃) 5.35 (s, 2H), 5.11-4.97 (m, 1H), 4.30-3.60 (m, 5H), 2.44 (q, J=7.6 Hz, 2H), 2.34 (t, J=7.6 Hz, 2H), 2.06-1.70 (m, 5H), 1.65-1.56 (m, 8H), 1.50-1.30 (m, 16H), 1.26-1.19 (m, 26H), 1.11-0.85 (m, 20H), 0.82 (s, 3H), 0.62-0.67 (m, 4H); LC-ELSD/MS ESI calcd. for C₅₃H₉₄O₈Na [M+Na]⁺ 881.9. found 881.9.

We claim:

1. A compound of Formula (I): 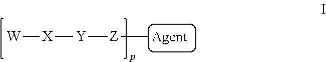
or a pharmaceutically acceptable salt thereof, wherein:
W is 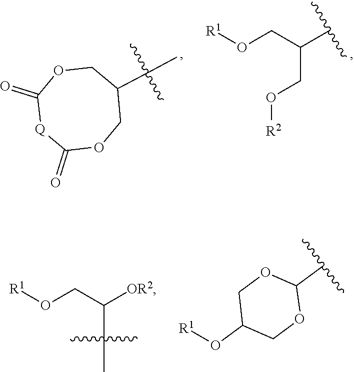
or —C(O)N(R³)₂;
Q is —C₁₋₈-alkylene-;
each of R¹ and R² is independently hydrogen, an acid-labile group, a lipid, or —C(O)R³;
each R³ is independently an optionally substituted C₁₋₄₀ aliphatic;
X is —O—, —NR—, —S—, —O(C₁₋₆ aliphatic)-O—, —O(C₁₋₆ aliphatic)-S—, —O(C₁₋₆ aliphatic)-NR—, —S(C₁₋₆ aliphatic)-O—, —S(C₁₋₆ aliphatic)-S—, —S(C₁₋₆ aliphatic)-NR—, —NR(C₁₋₆ aliphatic)-O—, —NR(C₁₋₆ aliphatic)-S—, —NR(C₁₋₆ aliphatic)-NR—, or —(C₁₋₆ aliphatic)-, wherein 0-2 methylene units of the C₁₋₆ aliphatic group are independently and optionally replaced with —O—, —NR—, or —S—, and wherein each instance of C₁₋₆ aliphatic is independently and optionally substituted with 1-3 deuterium or halogen;
each R is independently hydrogen, or an optionally substituted group selected from the group consisting of C₁₋₆ aliphatic, 3-8 membered carbocycle, C₆₋₁₀ aryl, 4-8 membered heterocycle comprising 1-2 heteroatoms independently selected from the group consisting of N, O and S, and 5-10 membered heteroaryl comprising 1-4 heteroatoms independently selected from the group consisting of N, O, and S;
Y is absent, —C(O)—, —C(NR)—, or —C(S)—;
Z is absent or an optionally substituted bivalent C₁₋₃₀ aliphatic, wherein 0-8 methylene units of Z are independently replaced by —R⁸—, —O—, —NR—, —S—, —OC(O)—, —C(O)O—, —C(O)—, —S(O)—, —S(O)₂—, —C(S)—, —OS(O)₂—, —S(O)₂O—, —N(R)S(O)₂—, —S(O)₂NR—, —N(R)C(O)—, —C(O)NR—, —OC(O)NR—, —N(R)C(O)O—, or an amino acid; and wherein one methylene unit of Z is optionally replaced with -M-; or
Z is selected from the group consisting of —C(R^{4a})(R^{4b})C(O)-M-, —C(R^{4a})(R^{4b})-M-, —C(R^{4a})(R^{4b})C(R^{5a})(R^{5b})(CH₂)ₙC(O)-M-, —C(R^{4a})(R^{4b})C(R^{5a})(R^{5b})(CH₂)ₙ-M-, —(CH₂)ₘC(R^{4a})(R^{4b})(CH₂)ₙC(R^{5a})(R^{5b})(CH₂)ₘC(O)-M-, and —(CH₂)ₘC(R^{4a})(R^{4b})(CH₂)ₙC(R^{5a})(R^{5b})(CH₂)ₘ-M-;
wherein either side of Z may be attached to Agent;
each —R⁸— is independently an optionally substituted bivalent group selected from the group consisting of C₃₋₆ carbocycle, C₆₋₁₀ aryl, 3-6 membered heterocycle comprising 1-4 heteroatoms independently selected from the group consisting of N, O, and S, and 5-10 membered heteroaryl comprising 1-4 heteroatoms independently selected from the group consisting of N, O, and S;
each R^{4a}, R^{4b}, R^{5b} and R^{5b} is independently selected from the group consisting of hydrogen, deuterium, halogen, —CN, —OR, —NR₂, —SR, —R⁹, and —R^{1′}; or
R^{4a} and R^{4b} or R^{5a} and R^{5b} together with the carbon atom to which they are attached, form a C₃₋₆ carbocycle or a 3-6 membered heterocycle comprising 1-2 heteroatoms independently selected from the group consisting of N, O and S;
each R⁹ is independently selected from the group consisting of C₃₋₈ carbocycle, C₆₋₁₀ aryl, 4-8 membered heterocycle comprising 1-2 heteroatoms independently selected from the group consisting of N, O, and S, and 5-10 membered heteroaryl comprising 1-4 heteroatoms independently selected from the group consisting of nitrogen, oxygen, and sulfur;
each R⁹ is independently selected from the group consisting of C₁₋₆ aliphatic optionally substituted with 1 to 6 —CN, —OR, —NR₂, —SR, —R⁹, deuterium or halogen;
-M- is a cleavable moiety;
each n is independently 0-18;
each m is independently 0-6;
p is 1 or 2; and
Agent is selected from the group consisting of:

| Agent | |
|---|---|
| No. | Structure |
| 1 | 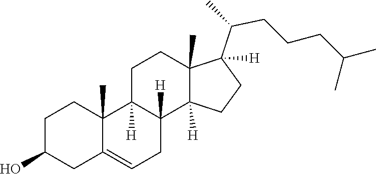 |
| 2 | 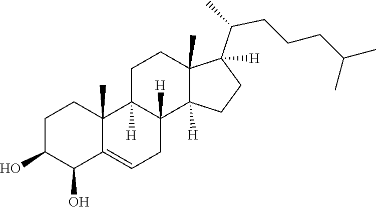 |
| 3 | 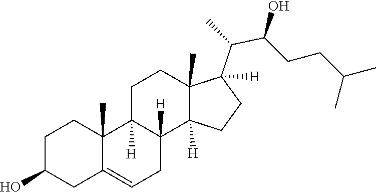 |
| 4 | 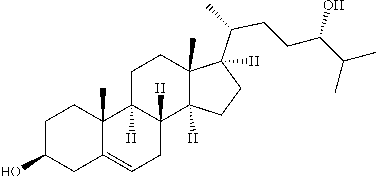 |
| 5 | 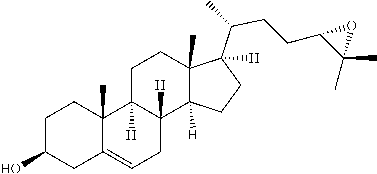 |
| 6 | 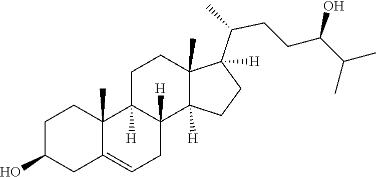 |
| 7 | 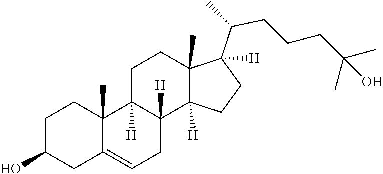 |
| 8 | 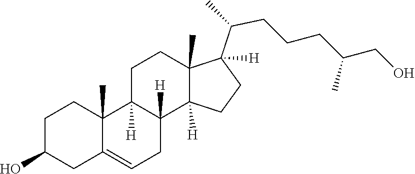 |
| 9 | 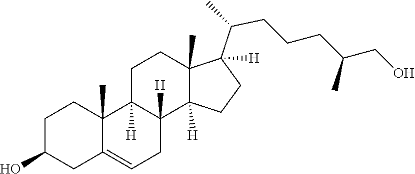 |
| 10 | 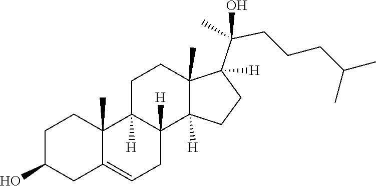 |
| 11 | 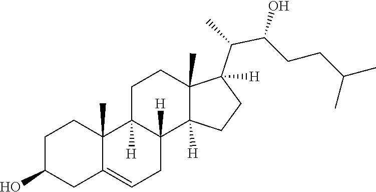 |
| 12 | 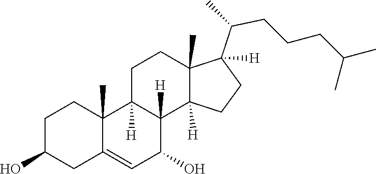 |
| 13 | 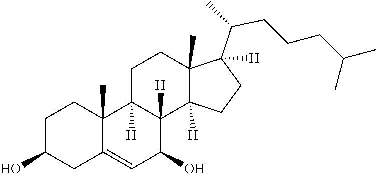 |
| 14 | 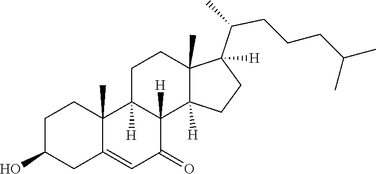 |
| 15 | 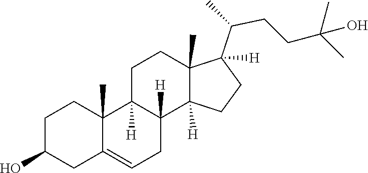 |
| 16 | 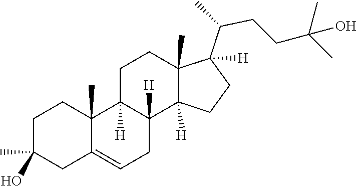 |
| 17 | 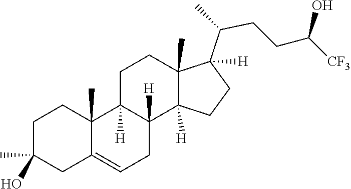 |
| 18 | 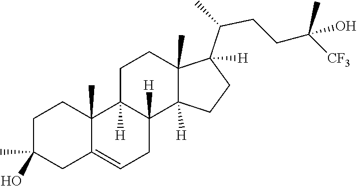 |
| 19 | 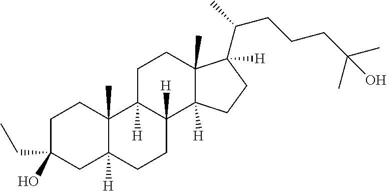 |
| 20 | 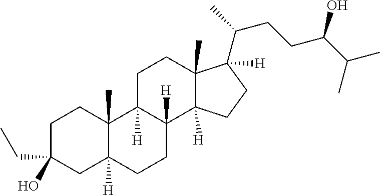 |
| 21 | 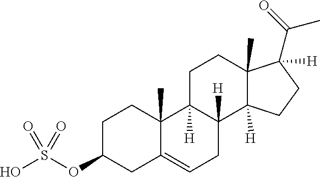 |
| 22 | 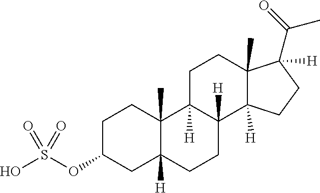 |
| 23 | 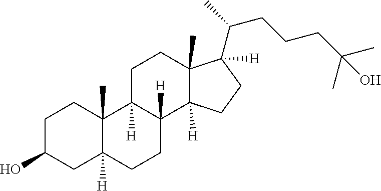 |
| 24 | 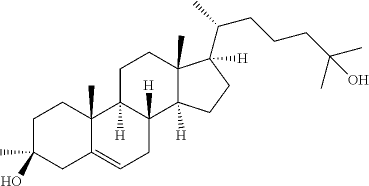 |
| 25 | 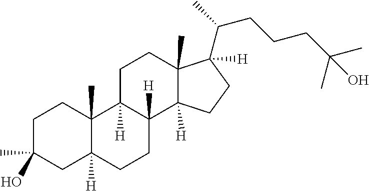 |
| 26 | 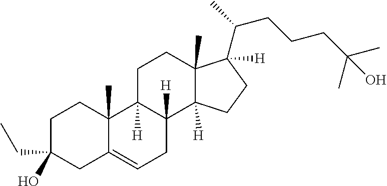 |
| 27 | 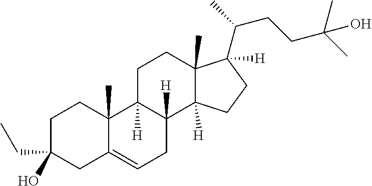 |

and deuterated variants thereof; wherein the point(s) of attachment of Agent to Z replaces one or more hydrogen atoms on the Agent.

2. The compound according to claim 1, or a pharmaceutically acceptable salt thereof, wherein W is

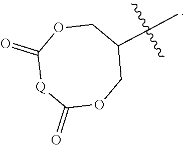

3. The compound according to claim 1, or a pharmaceutically acceptable salt thereof, wherein W is

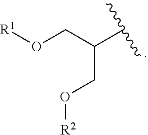

4. The compound according to claim 1, or a pharmaceutically acceptable salt thereof, wherein W is

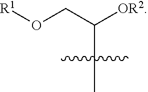

5. The compound according to claim 1, or a pharmaceutically acceptable salt thereof, wherein W is

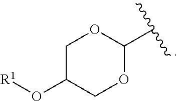

6. The compound according to claim 1, or a pharmaceutically acceptable salt thereof, wherein W is —C(O)N—(R³)₂.

7. The compound according to claim 1 or 2, wherein Q is —C₅alkylene-.

8. The compound according to claim 1, 3, 4, or 5, or a pharmaceutically acceptable salt thereof, wherein each of R and R² is independently —C(O)R³.

9. The compound according to any one of claims 1, 6, or 8, or a pharmaceutically acceptable salt thereof, wherein each R³ is independently C₁₋₄₀ aliphatic.

10. The compound according to any one of claims 1-9, or a pharmaceutically acceptable salt thereof, wherein X is —O—.

11. The compound according to any one of claims 1-9, or a pharmaceutically acceptable salt thereof, wherein X is —(C₁₋₆ aliphatic)-.

12. The compound according to any one of claims 1-11, or a pharmaceutically acceptable salt thereof, wherein each R is independently hydrogen, C₁₋₆ aliphatic, 3-8 membered carbocycle, C₆₋₁₀ aryl, 4-8 membered heterocycle comprising 1-2 heteroatoms independently selected from the group consisting of N, O and S, or 5-10 membered heteroaryl comprising 1-4 heteroatoms independently selected from the group consisting of N, O, and S;

13. The compound according to any one of claims 1-12, or a pharmaceutically acceptable salt thereof, wherein Y is —C(O)—.

14. The compound according to any one of claims 1-13, or a pharmaceutically acceptable salt thereof, wherein Z is an optionally substituted bivalent C₁₋₃₀ aliphatic, wherein 0-8 methylene units of Z are independently replaced by —R⁸—, —O—, —NR—, —S—, —OC(O)—, —C(O)O—, —C(O)—, —S(O)—, —S(O)₂—, —C(S)—, —N(R)S(O)₂—, —S(O)₂NR—, —N(R)C(O)—, —C(O)NR—, —OC(O)NR—, —N(R)C(O)O—, or an amino acid; and wherein 1 methylene unit of Z is optionally replaced with -M-.

15. The compound according to claim 14, or a pharmaceutically acceptable salt thereof, wherein the amino acid is selected from the group consisting of

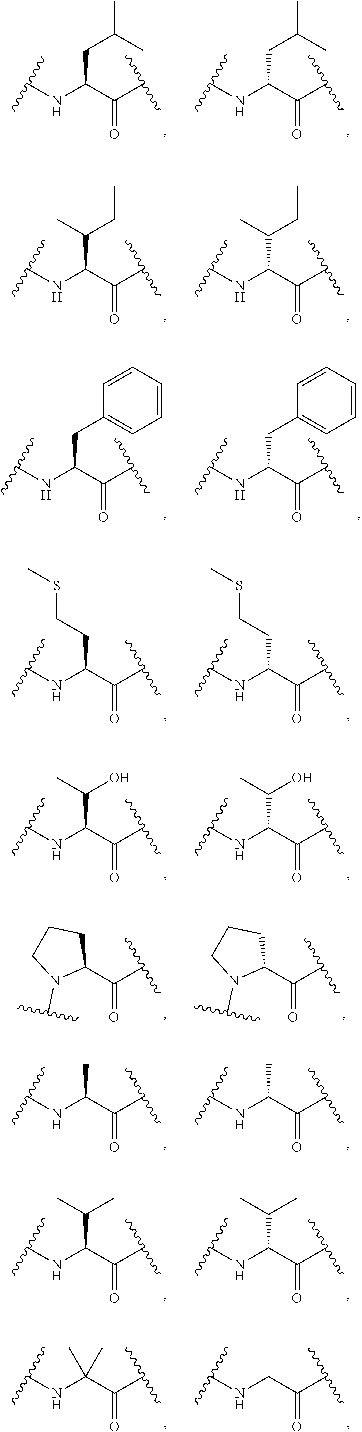

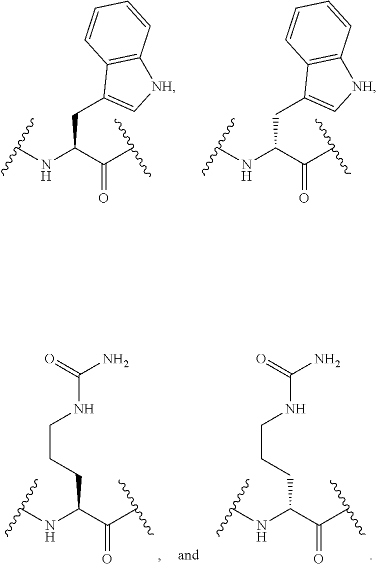 [SMILES: *N[C@@H](Cc1c[nH]c2ccccc12)C(*)=O ; *N[C@H](Cc1c[nH]c2ccccc12)C(*)=O ; *N[C@@H](CCCNC(N)=O)C(*)=O ; *N[C@H](CCCNC(N)=O)C(*)=O]

16. The compound according to any one of claims 1-13, or a pharmaceutically acceptable salt thereof, wherein Z is selected from the group consisting of —CH(R^{4a})C(O)-M-, —CH(R^{4a})-M-, —CH(R^{4a})CH(R^{5a})C(O)-M-, —CH(R^{4a})CH(R^{5a})(CH₂)ₙC(O)-M-, —CH(R^{4a})CH(R^{5a})(CH₂)ₙ-M-, and —C(R^{4a})(R^{4b})(CH₂)~C(R^{5a})(R^{5b})C(O)-M-, wherein either side of Z may be attached to Agent.

17. The compound according to any one of claims 1-13, or a pharmaceutically acceptable salt thereof, wherein Z is absent.

18. The compound according to any one of claims 1-13, or a pharmaceutically acceptable salt thereof, wherein Z is C₁₋₂₅ alkylene optionally substituted with 1-4 substituents selected from the group consisting of deuterium, halogen, —CN, a 3-6 membered carbocycle, C₆₋₁₀ aryl, a 4-6 membered heterocycle comprising 1-2 heteroatoms independently selected from N, O and S, a 5-6 heteroaryl comprising 1-4 heteroatoms independently selected from N, O and S, or C₁₋₆ aliphatic optionally substituted with 1-6 deuterium or halogen; wherein 0-4 methylene units of Z are independently replaced by —O—, —OC(O)—, —C(O)O—, or —C(O)—; and 1 methylene unit of Z is optionally replaced with -M-.

19. The compound according to any one of claims 1-18, or a pharmaceutically acceptable salt thereof, wherein -M- is selected from the group consisting of: 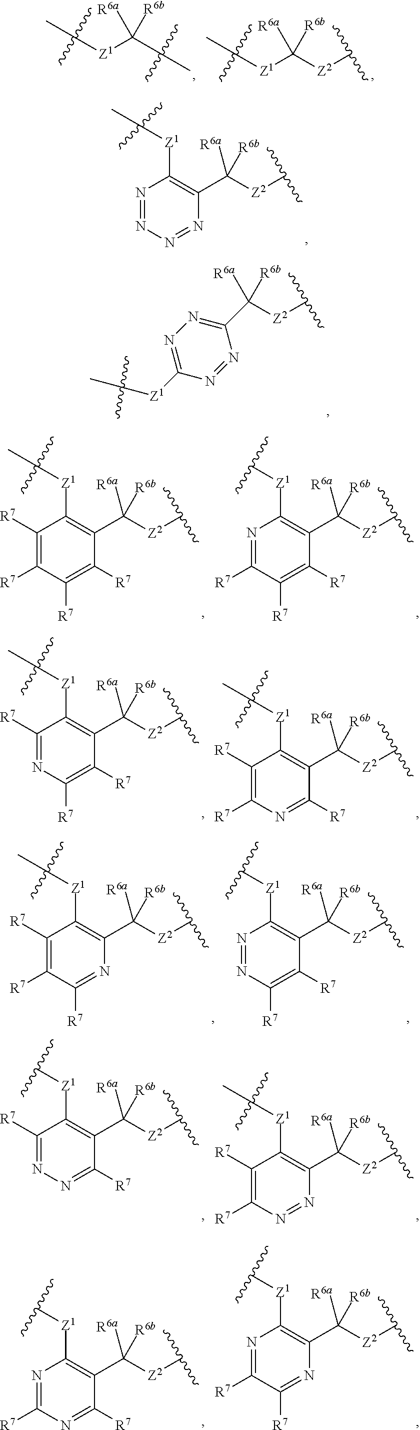 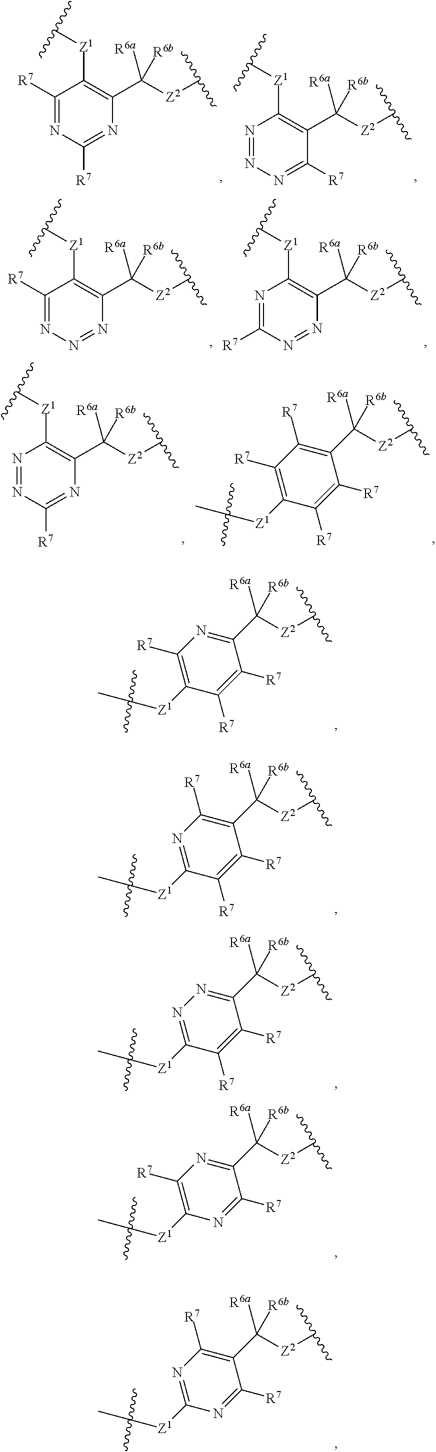 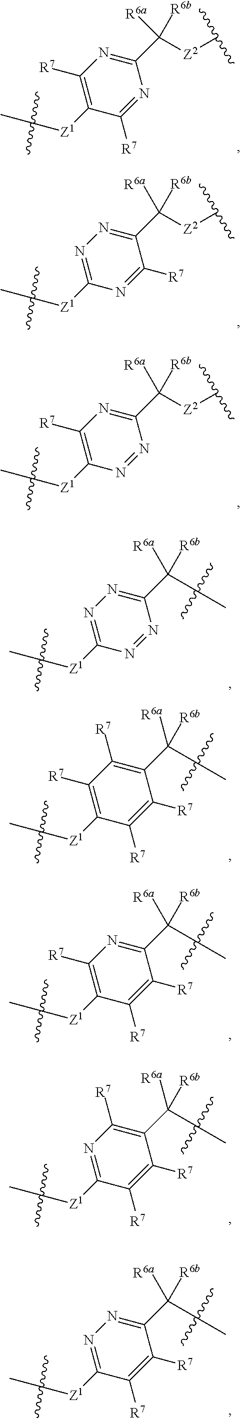 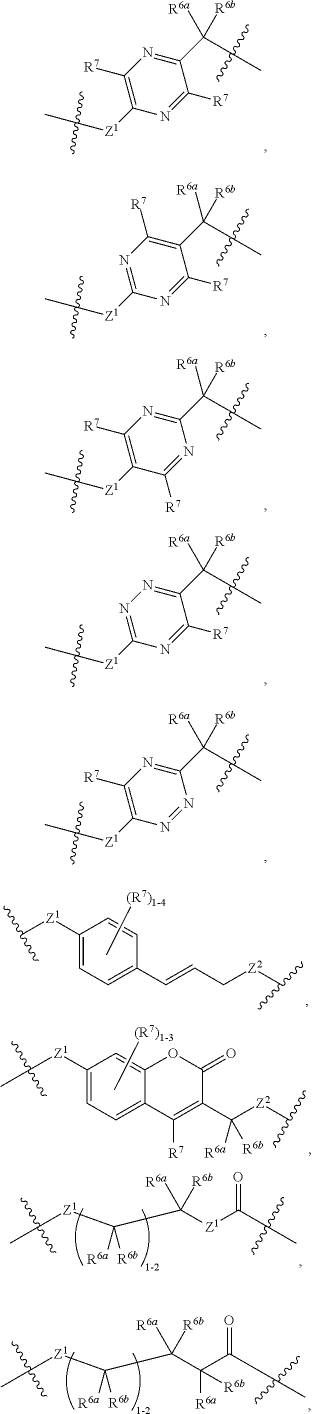 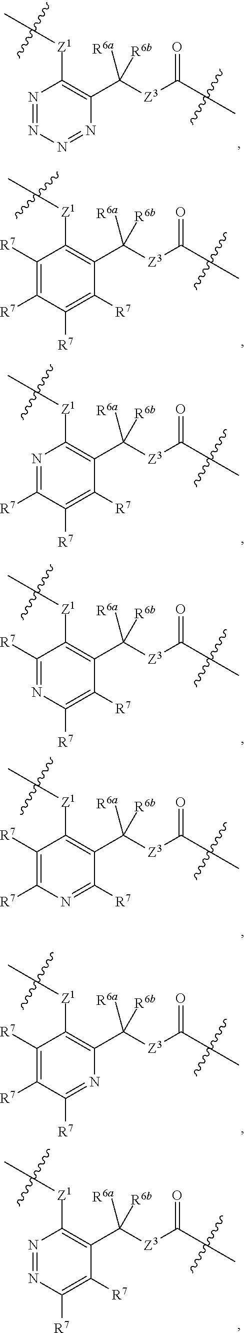 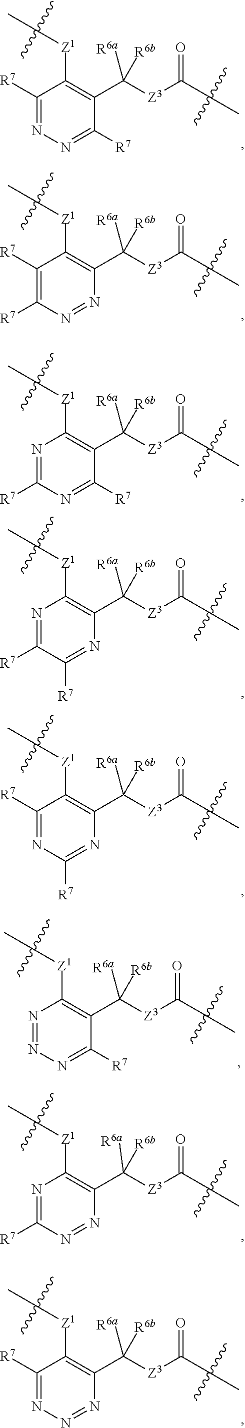 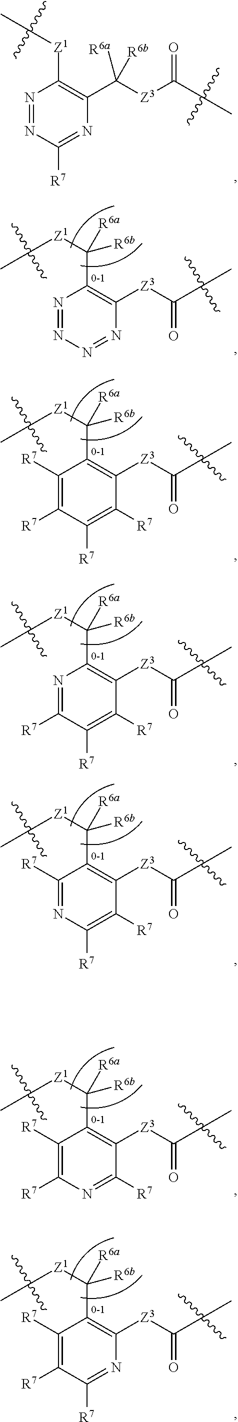 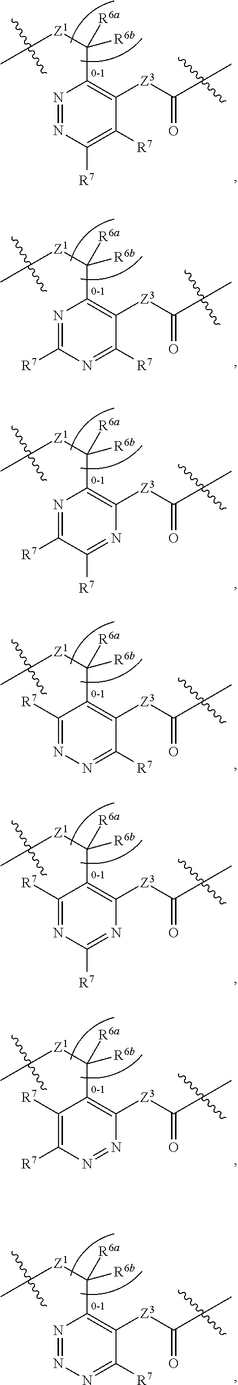 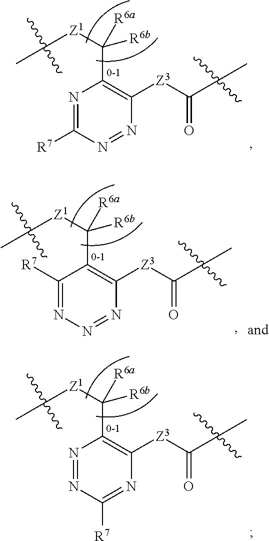
wherein either side of M may be attached to Agent; and
wherein
each of R^{6a} and R^{6b} is independently selected from the group consisting of hydrogen, deuterium, C₁₋₅ aliphatic, halogen, and —CN;
each R⁷ is independently selected from the group consisting of hydrogen, deuterium, halogen, —CN, —OR, —NR₂, —NO₂, —SR, —R⁹, or —R¹⁰;
each Z¹ is independently selected from the group consisting of —O—, —NR—, and —S—;
each Z² is independently selected from the group consisting of —O—, —NR—, —S—, —OC(O)—, —N(R)C(O)O—, and —OC(O)NR—; and
each Z³ is independently absent or selected from the group consisting of —O—, —NR—, —S—, and —C(R^{6a})(R^{6b})—.

20. The compound according to claim 19, or a pharmaceutically acceptable salt thereof, wherein -M- is selected from the group consisting of: 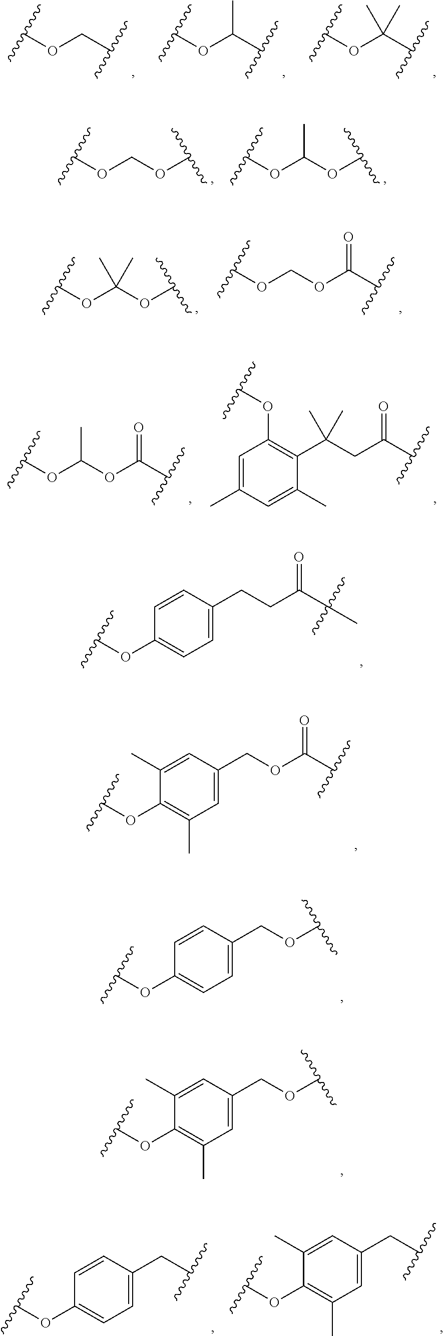 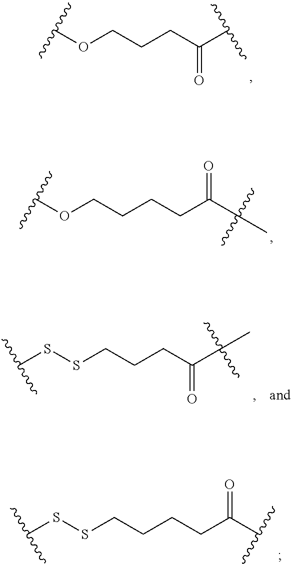 [SMILES: *OCCCC(*)=O ; *OCCCCC(*)=O ; *SSCCCC(*)=O ; *SSCCCCC(*)=O]
wherein either side of M may be attached to Agent.

21. The compound according to any one of claims 1-20, or a pharmaceutically acceptable salt thereof, wherein each of R^{4a} and R^{4b} is independently hydrogen, deuterium, halogen, —CN, or C₁₋₄ aliphatic optionally substituted with 1-6 deuterium or halogen atoms; or R^{4a} and R^{4b} together with the carbon atom to which they are attached, form a C₃₋₆ carbocycle or a 3-6 membered heterocycle comprising 1-2 heteroatoms independently selected from the group consisting of N, O and S.

22. The compound according to any one of claims 1-20, or a pharmaceutically acceptable salt thereof, wherein each of R^{5a} and R^{5b} is independently hydrogen, deuterium, halogen, —CN, or C₁₋₄ aliphatic optionally substituted with 1-6 deuterium or halogen atoms; or R^{5a} and R^{5b} together with the carbon atom to which they are attached, form a C₃₋₆ carbocycle or a 3-6 membered heterocycle comprising 1-2 heteroatoms independently selected from the group consisting of N, O and S.

23. The compound according to any one of claims 1-22, or a pharmaceutically acceptable salt thereof, wherein each of R^{4a}, R^{4b}, R^{5a} and R^{5b} is independently hydrogen or C₁₄ alkyl optionally substituted with 1-6 deuterium or halogen atoms.

24. The compound according to any one of claims 1-22, or a pharmaceutically acceptable salt thereof, wherein p is 1.

25. The compound according to any one of claims 1-22, or a pharmaceutically acceptable salt thereof, wherein p is 2.

26. The compound according to any one of claims 1-25, or a pharmaceutically acceptable salt thereof, wherein 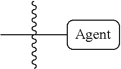
is a structure as set forth in Table 1, pharmaceutically acceptable salts thereof, deuterated variants thereof, or a combination thereof.

27. The compound according to claim 1, or a pharmaceutically acceptable salt thereof, wherein the compound is selected from any one of compounds 1-2201, or pharmaceutically acceptable salts thereof, deuterated variants thereof, or combination thereof.

28. A pharmaceutical composition comprising the compound according to any of claims 1-27, or a pharmaceutically acceptable salt thereof, and a pharmaceutically acceptable carrier.

29. The pharmaceutical composition according to claim 28, wherein said composition further comprises an additional therapeutic agent.

30. A method of treating a disease, disorder, or condition requiring allosteric NMDA receptor modulation in a subject, comprising administering to the subject an effective amount of a compound according to any one of claims 1-27 or a pharmaceutically acceptable salt thereof or a pharmaceutical composition according to any one of claim 28 or 29.

31. The method of claim 30, wherein the disease, disorder, or condition requiring allosteric NMDA receptor modulation is a CNS-related condition.

32. The method of claim 31, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

33. The compound according to any one of claims 1-27, or a pharmaceutically acceptable salt thereof for use in treating a disease, disorder, or condition requiring allosteric NMDA receptor modulation.

34. The compound or a pharmaceutically acceptable salt thereof for use according to claim 33, wherein the disease, disorder, or condition requiring allosteric NMDA receptor modulation is a CNS-related condition.

35. The compound or a pharmaceutically acceptable salt thereof for use according to claim 34, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

36. A composition comprising the compound according to any one of claims 1-27, or a pharmaceutically acceptable salt thereof for use in treating a disease, disorder, or condition requiring allosteric NMDA receptor modulation.

37. The composition for use according to claim 36, wherein the disease, disorder, or condition requiring allosteric NMDA receptor modulation is a CNS-related condition.

38. The composition for use according to claim 37, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

39. Use of a compound according to any one of claims 1-27, or a pharmaceutically acceptable salt thereof for the manufacture of a medicament for treating a disease, disorder, or condition requiring allosteric NMDA receptor modulation.

40. The use according to claim 39, wherein the disease, disorder, or condition requiring allosteric NMDA receptor modulation is a CNS-related condition.

41. The use according to claim 40, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.
